(19) United States
(12) Patent Application Publication
(10) Pub. No.: US 2022/0144838 A1
(43) Pub. Date: May 12, 2022

(54) Compounds as Inhibitors of Macrophage Migration Inhibitory Factor

(71) Applicants: Kin Chiu FONG, San Diego, CA (US); Hongyu YANG, Shanghai (CN); Ping DU, San Francisco, CA (US); Jinfu YANG, Foster city, CA (US); NANJING IMMUNOPHAGE BIOTECH CO., LTD., Nanjing (CN)

(72) Inventors: Kin Chiu FONG, San Diego, CA (US); Hongyu YANG, Shanghai (CN); Ping DU, San Francisco, CA (US); Jinfu YANG, Foster City, CA (US)

(73) Assignee: NANJING IMMUNOPHAGE BIOTECH CO., LTD., Nanjing (CN)

(21) Appl. No.: 17/438,413

(22) PCT Filed: Mar. 13, 2020
(86) PCT No.: PCT/US2020/022768

§ 371 Date: Sep. 10, 2021

Related U.S. Application Data
(60) Provisional application No. 62/817,563, filed on Mar. 13, 2019.

(51) Int. Cl. C07D 473/16; A61P 35/00

(52) U.S. Cl. CPC C07D 473/16; A61P 35/00

(57) ABSTRACT

The present invention provides compounds of Formula (I) shown below and their pharmaceutically acceptable salt, solvates, isomers, or prodrugs, as well as pharmaceutical compositions containing these compounds. Also provided by the invention is a method for treating a disorder mediated by macrophage migration inhibitory factor in a subject, comprising administering to the subject in need thereof a compound or a pharmaceutical composition of this invention.

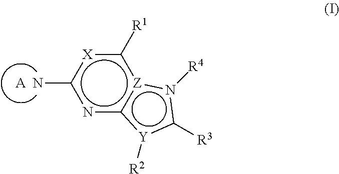

### CROSS-REFERENCE TO RELATED APPLICATION

This application claim priority to U.S. Application No. 62/817,563, filed on Mar. 13, 2019, the contents of which are incorporated herein by reference in their entirety.

### FIELD OF THE INVENTION

The present invention concerns novel inhibitors of macrophage migration inhibitory factor (MIF) and their use in the treatment of diseases mediated by MIF.

### BACKGROUND OF THE INVENTION

Macrophage migration inhibitory factor (MIF) is a cytokine originally found to play a role in inhibiting macrophage migration. Unlike other cytokines, MIF has enzymatic activities and shares characteristics of endocrine molecule and chaperone-like protein. MIF binds to its receptor CD74, which forms a complex with CD44 to transduce intracellular signaling. Simultaneously, MIF also binds to the chemokine receptors CXCR2 and CXCR4 to activate down-stream signaling, including ERK1/2 and PI3K. MIF exerts pleiotropic biologic activities that include glucocorticoid antagonism, up-regulation of Toll-like receptor 4 expression, control of transcriptional effects of JAB1, and suppression of activation-induced, p53-dependent apoptosis, by its direct interaction with p53, and stabilization of p53-MDM2 complex. This latter action may sustain inflammatory responses in spite of activation-induced apoptosis, and it may mediate MIF broad inflammatory and proliferative effects on diverse cell types. MIF has originally attracted much attention as a central mediator of several inflammatory and autoimmune diseases. Increased production of MIF has been linked to a more aggressive course of inflammatory or autoimmune diseases, such as, asthma and rheumatoid arthritis.

Recent studies high-light its role in tumorigenesis, such as angiogenesis, cell proliferation, and tumor invasion. In agreement with these oncogenic properties, both experimental and clinical studies have shown that high levels of MIF are found in several types of human cancers and are apparently implicated in all stages of development of the tumors. Upregulated MIF expression has been reported in gastric cancer, pancreatic cancer, melanoma, hepatocellular carcinoma, malignant glioma and cervical adenocarcinoma. The important role of MIF in tumorigenesis has been proved by experiments showing that genetic deletion or pharmacological inhibition of MIF prevents tumor cell proliferation in vitro or tumor growth in vivo. Moreover, recent studies have demonstrated that MIF may favor the generation of an oncogenic environment by favoring the escape of tumors from immune surveillance, via induction of myeloid-derived suppressor cells in the tumor microenvironment, inhibition of T lymphocyte activation, polarization of macrophages to an M2 phenotype, and inhibition of natural killer (NK) cells.

Given the role that MIF plays a role in the pathogenesis of various diseases, it is desirable to prepare compounds that inhibit MIF activity, which may be used in the treatment of diseases mediated by MIF.

### BRIEF SUMMARY OF THE INVENTION

In one aspect, the present invention provides compounds of Formula (I) shown below, as well as their pharmaceutically acceptable salts, solvents, isomers, or prodrugs.

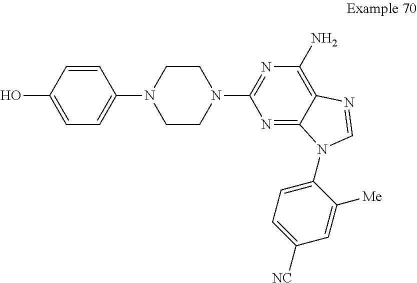 [SMILES: Cc1cc(C#N)ccc1-n1cnc2c(N)nc(N3CCN(c4ccc(O)cc4)CC3)nc21]

### In Formula (I),

R¹ is absent, H, alkyl, halo, hydroxyl, alkoxy, thio, thiol ether, monocyclic or bicyclic aryl, monocyclic or bicyclic heteroaryl, monocyclic or bicyclic cycloalkyl, monocyclic or bicyclic heterocycloalkyl, arylalkyl, heteroaryl-alkyl, amino, or hydroxylamino, wherein alkyl, aryl, heteroaryl, cycloalkyl, heterocycloalkyl, arylalkyl, heteroaryl-alkyl, or amino is optionally substituted with one or more substituents each independently selected from the group consisting of halo, cyano, hydroxyl, alkyl, hydroxyalkyl, cycloalkyl, alkoxy, aryl, heteroaryl, alkylcarbonyl, alkoxycarbonyl, alkoxycarbonylalkyl, hydroxycarbonyl, hydroxycarbonylalkyl, hydroxyalkylcarbonyl, hydroxyalkylcarbonylalkyl, alkoxyalkylcarbonylalkyl, aminocarbonylalkyl, amido, hydroxyalkyl, cycloalkyl-alkyl, arylalkyl and arylalkylhydroxyalkyl, wherein one —CH₂— group in the foregoing heterocycloalkyl is optionally replaced with carbonyl group C═O;

R² is absent, H, halo, cyano, hydroxyl, alkoxy, alkyl, cycloalkyl, heterocycloalkyl, amino, thiol ether, arylalkyl, heteroaryl-alkyl, aryl, or heteroaryl, wherein alkyl, cycloalkyl, heterocycloalkyl, aryl, or heteroaryl is substituted with one or more substituents each independently selected from the group consisting of halo, cyano, alkyl, alkenyl, alkynyl, hydroxyl, alkoxy, hydroxycarbonyl, alkoxycarbonyl, boronic acid, boronic ester, phosphonyl, aryl, heteroaryl, aryl-heteroaryl, heteroaryl-aryl, hydroxyaryl-heteroaryl, hydroxyheteroaryl-aryl, amino, aminocarbonyl (amido), alkylsulfonylaminocarbonyl, cycloalkyl-sulfonylaminocarbonyl, alkylsulfonyl, heteroaryl, hydroxycarbonylalkylaminocarbonyl, cycloalkyl, heterocycloalkyl, thiol ether, and aryl;

each of R³ and R⁴, independently, is H or alkyl optionally substituted with one or more substituents each independently selected from the group consisting of halo, cyano, amino, hydroxyl, alkoxy, thiol ether, alkylcarbonyl, hydroxycarbonyl, alkoxycarbonyl, aryl, heteroaryl, cycloalkyl, heterocycloalkyl, and arylalkyl;

X is N or CR⁵, Y is N or C, Z is N or C, provided that when Z is N, X is CR⁵ and Y is C, and when Z is C, X is N and Y is N;

R⁵ is H or alkyl optionally substituted with one or more substituents each independently selected from the group consisting of halo, cyano, amino, hydroxyl, and alkoxy;

Ring A, as represented by the moiety

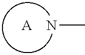

in Formula (I), includes the nitrogen atom shown in Figure (I) and is a heteroaryl or heterocyclic group, and Ring A is optionally substituted with one or more substituents each independently selected from the group consisting of aryl, alkynyl, heteroaryl, cycloalkyl, heterocyclic, halo, cyano, alkyl, alkoxycarbonyl, hydroxyl, acyl, aminocarbonyl, alkylsulfonyl, and oxo, wherein aryl, heteroaryl, cycloalkyl, heterocyclic, alkyl, alkynyl, acyl, aminocarbonyl or alkylsulfonyl is optionally substituted with one or more substituents each independently selected from the group consisting of hydroxyl, alkoxy, hydoxyalkyl, amino, aminoalkyl, hydroxycarbonyl, alkoxycarbonyl, hydroxycarbonylalkyl, aminocarbonyl, aminocarbonylalkyl, cycloalkyl, heterocyclic, halo or alkyl;

wherein one —CH₂— group in the foregoing heterocyclic group is optionally replaced with carbonyl group C═O.

In some embodiments, R¹ is H, alkyl, monocyclic or bicyclic heteroaryl, monocyclic or bicyclic aryl, fused arylheterocycloalkyl, fused heterocycloalkylaryl, amino, arylamino, heteroarylamino, arylalkylamino, heteroarylalkylamino, heterocycloalkyl, or (cycloalkyl)amino, wherein one —CH₂— group in the foregoing heterocycloalkyl is optionally replaced with carbonyl group (C═O), and R¹ is optionally substituted with one or more substituents each independently being halo, alkyl, alkenyl, cycloalkyl, hydroxyl, hydroxyalkyl, alkoxy, amino, cyano, hydroxycarbonyl, hydroxycarbonylalkyl, alkoxycarbonyl, alkoxycarbonylalkyl, aminocarbonyl, alkylaminocarbonyl, hydroxycarbonylalkyl, hydroxycarbonylalkenyl, arylalkylhydroxyalkyl or heteroaryl.

In some other embodiments, the heteroaryl in R¹ is indolinyl, isoindolinyl, indolinonyl, isoindolinonyl, indazolyl, dihydroindenyl, benzotriazolyl, pyridinyltriazolyl, indazolyl, indolyl, pyrrolopyridinyl, or benzoimidazolyl, and the heteroaryl is optionally substituted with one or more substituents each independently being alkyl, hydroxyalkyl, alkoxy, halo, hydroxyl, amino, cyano, hydroxycarbonyl, alkoxycarbonyl, alkylaminocarbonyl, hydroxycarbonlyalkenyl, hydroxycarbonlyalkyl, heteroaryl, or cycloalkyl.

Some examples of suitable R¹ include

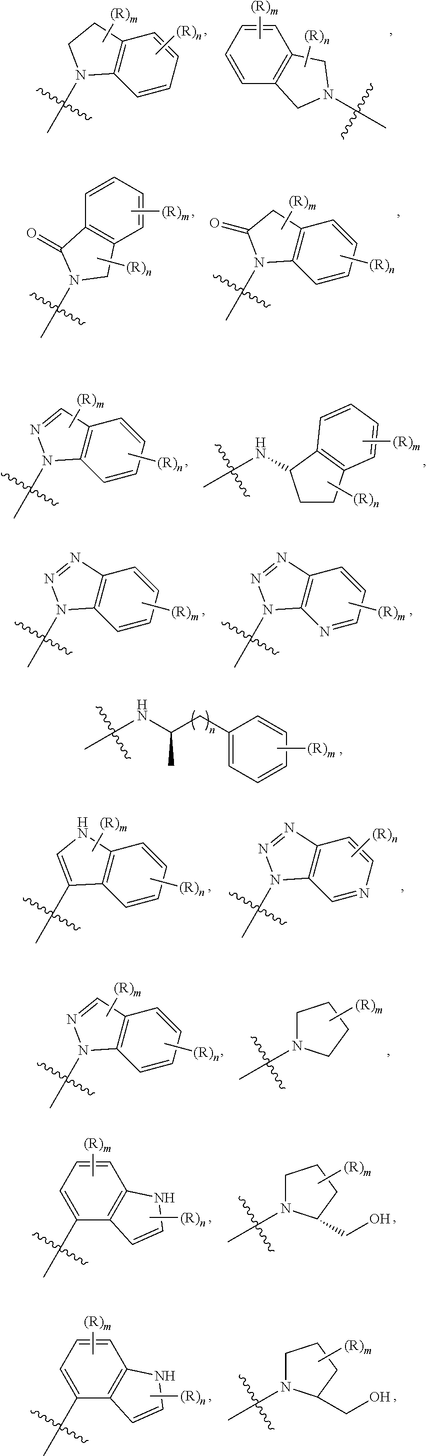

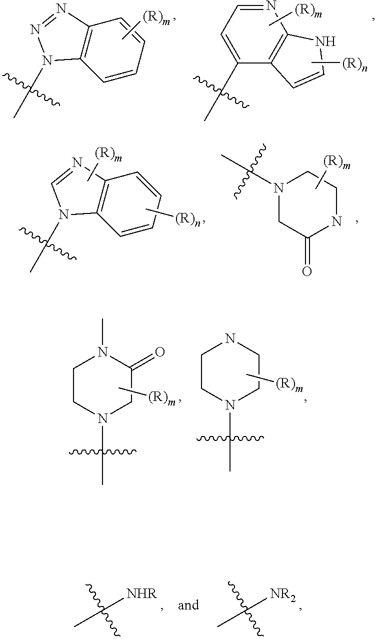

wherein each of m and n is independently 0, 1, or 2; and each R independently is alkyl, cycloalkyl, hydroxyalkyl, alkoxy, halo, amino, cyano, hydroxycarbonyl, alkoxycarbonyl, alkylaminocarbonyl, hydroxycarbonylalkenyl, hydroxycarbonylalkyl, heteroaryl, or aryl.

Some other examples of R¹ include H, alkyl, amino,

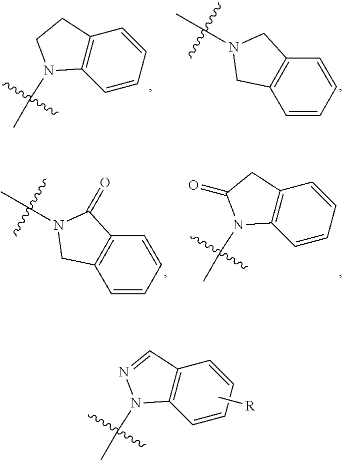

(wherein R is —CH₂OH, —OCH₃, Cl, —OH, —NH₂, —CN, or —COOH),

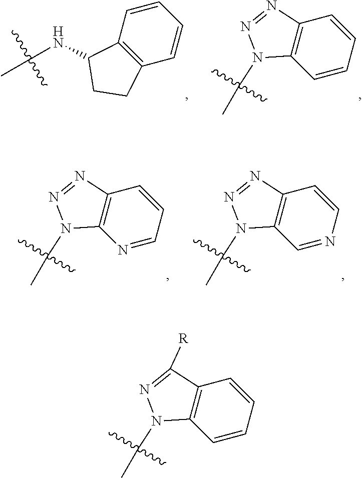

(wherein R is —COOH, —COOEt, —CONHCH₃, —CH₂OH, —CH═CHCOOH, —CH₂CH₂COOH, —CN, -heteroaryl),

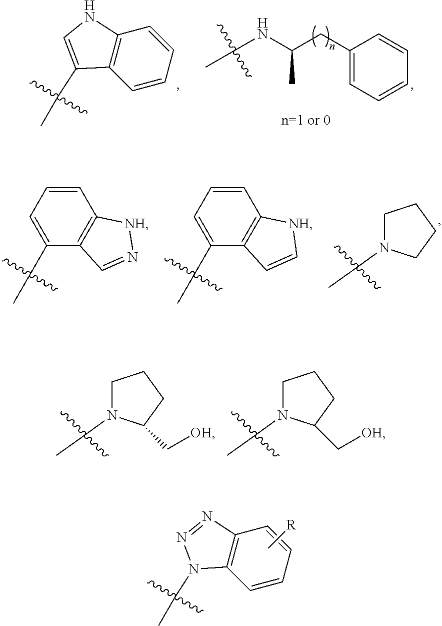

(R is —CH₃, Cl, —COOH, —CH₂OH),

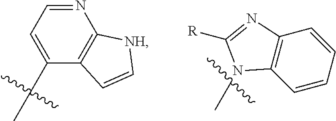

(R is H, —CH₃, —CH₂OH),

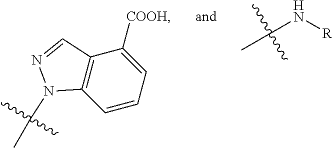

(R is H, hydroxylalkyl, phenylalkyl, cycloalkyl, or cycloalkylalkyl).

In some embodiments, R² is H, halo, heterocycloalkyl, or alkyl optionally substituted with one or more substituents each independently being amino, aryl, hydroxyl, hydroxycarbonyl, alkoxycarbonyl, aminocarbonyl, arylalkylsulphonylaminocarbonyl, alkylsulphonylaminocarbonyl, cycloalkylsulphonylaminocarbonyl, arylsulphonylaminocarbonyl, alkylcarbonylamino, heteroaryl, or hydroxycarbonylalkylalkylaminocarbonyl; or R² is aryl or heteroaryl and is optionally substituted with one or more substituents each independently being halo, alkoxy, alkyl, OH, CN, aminocarbonyl, hydroxycarbonyl, alkoxycarbonyl, aryl, heterocycloalkyl, or arylheterocycloalkyl.

In some other embodiments, R² is phenyl or heteroaryl and is optionally substituted with one or more substituents each independently being alkoxy, alkyl, halo, OH, CN, aminocarbonyl, hydroxycarbonyl, alkoxycarbonyl, aryl, heterocyclicalkyl, or arylheterocyclicalkyl.

Some examples of suitable R² include H, halo, monohydroxyalkyl, dihydroxylalkyl, aminoalkyl, hydroxycarbonylalkyl, alkoxycarbonylalkyl, aminocarbonylalkyl, alkylcarbonylaminoalkyl, sulfonylaminocarbonylalkyl, alkylsulfonylaminocarbonylalkyl, cycloalkylsulfonylaminocarbonylalkyl, arylsulfonylaminocarbonylalkyl, arylalkylsulfonylaminocarbonylalkyl, phenylalkyl, tetrazolylalkyl, pyridinylalkyl, or pyrimidinylalkyl; or R² is phenyl, pyridinyl, pyrimidinyl, pyridazinyl, and pyrazolyl, and they can be optionally substituted with one or more substituents each independently being hydroxyl, halo, alkyl, alkoxy, cyano, phenyl, hydroxycarbonyl, alkoxycarbonyl, aminocarbonyl, alkylcarbonylaminoalkyl, alkylaminocarbonyl, or hydroxyphenylpiperazinyl.

Some other examples of suitable R² include halo, —(CH₂)₂OH, —CH(CH₂OH)₂, —(CH₂)₂COOH, —(CH₂)₂CONH₂, —CH₂COOH, —CH₂COOCH₃, —CH₂—CO—NH—CH₂—COOH, —CH(CH₃)COOH, —C(CH₃)₂—COOH, CH(CH₃)COOCH(CH₃)₃, CH(C₂H)COOH, —CH₂CH₂NH₂, —CH₂CONH₂, —CH₂CONHCH₃, —CH₂CONHC₂H₅, —CH(C₂H₅)CH₂OH, —CH₂CO—NH—SO₂CH₃, —CH₂CO—NH—SO₂C₂H₅, —CH₂CO—NH—SO₂CH(CH₃)₂, —(CH₂)₂—NH—SO₂—CH₃, —CH₂CO—NH—SO₂CH₂CH₂CH₃, (cyclopropyl)SO₂—NH—COCH₂—, phenyl-SO₂—NH—COCH₂—, benzyl-SO₂—NH—COCH₂—, tetrazolylmethyl, tetrazolylethyl, and pyridinylmethyl. Further examples of R² include phenyl, pyridinyl, pyrimidinyl, pyridazinyl, and pyrazolyl, and they can be optionally substituted with one or two substituents each independently being hydroxyl, halo, alkyl, alkoxy, cyano, phenyl, hydroxycarbonyl, aminocarbonyl, methylaminocarbonyl, or hydroxyphenylpiperazinyl.

In some embodiments, R³ is H; and R⁴ is H, hydroxycarbonylalkyl, or alkoxycarbonylalkyl.

In some embodiments, X is N, Y is N, Z is C, thereby making Formula (I) into Formula (II) shown below:

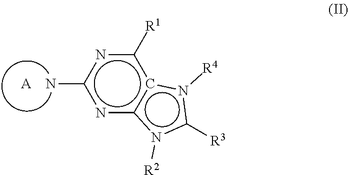

In some other embodiments, X is CR⁵, R⁵ is H or optionally substituted alkyl, Y is C, and Z is N, thereby making Formula (I) into Formula (III) shown below:

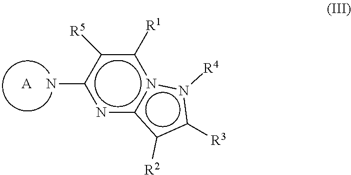

A specific example of R⁵ in Formula (III) is H.

In some embodiments of the compounds of this invention, Ring A is

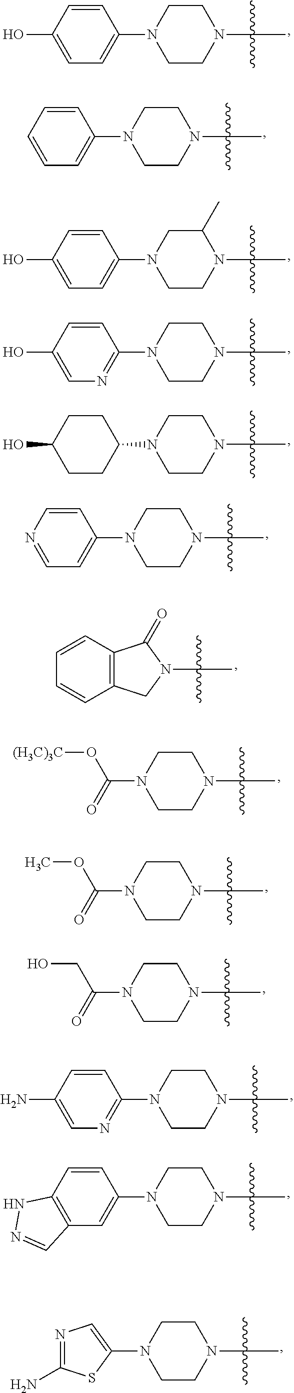

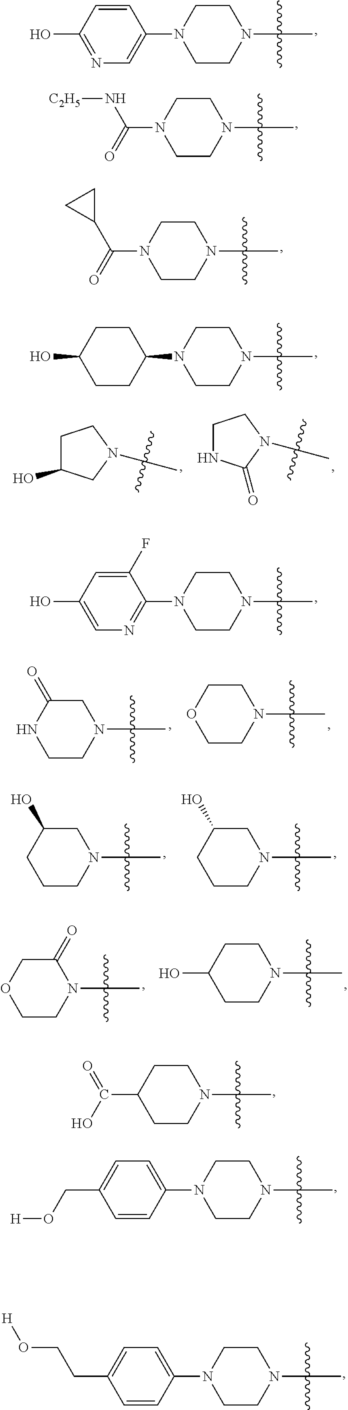

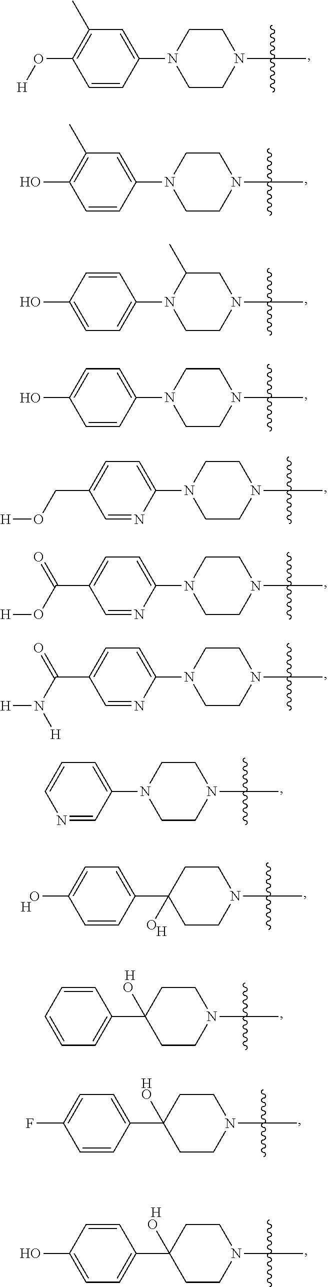

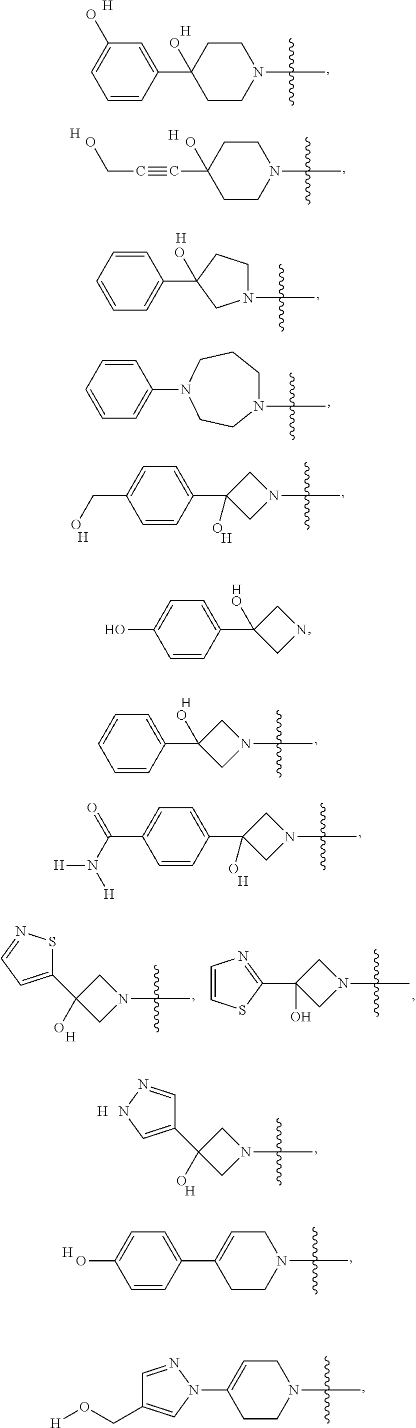

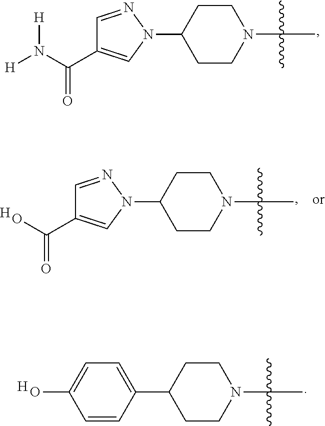

In yet still some other embodiments of the compounds of this invention, the compound of Formula (I) is

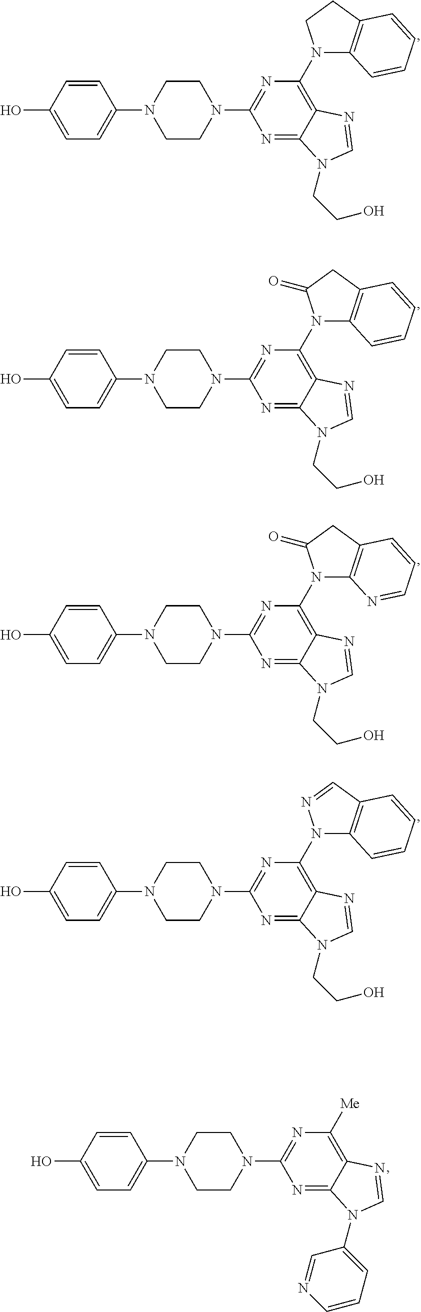

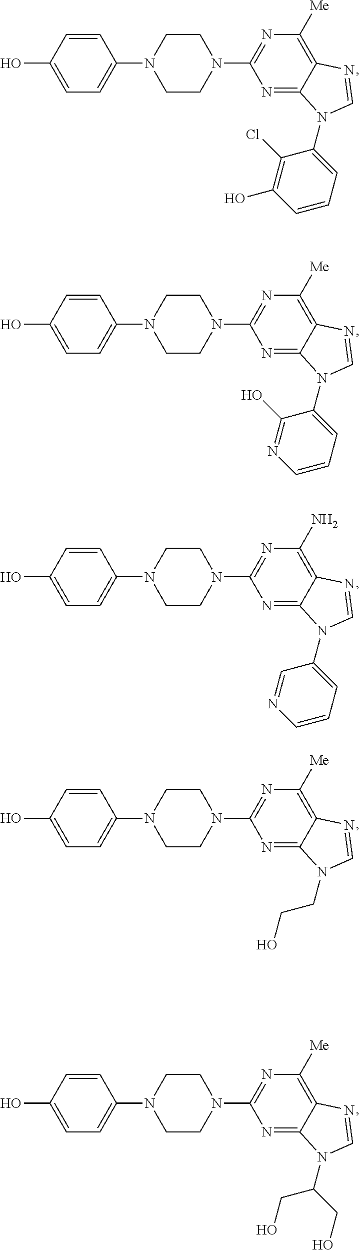

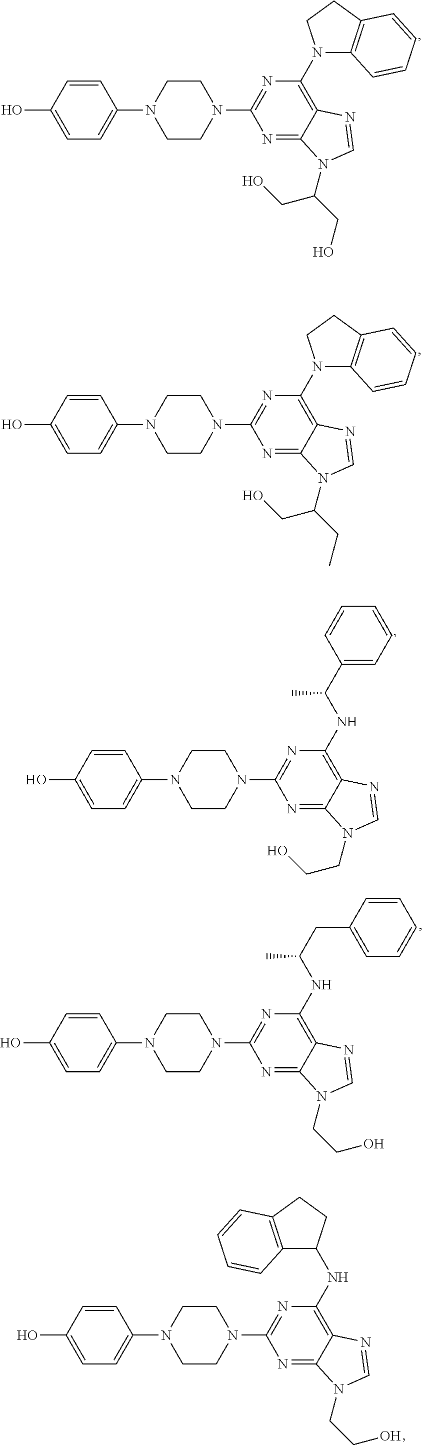

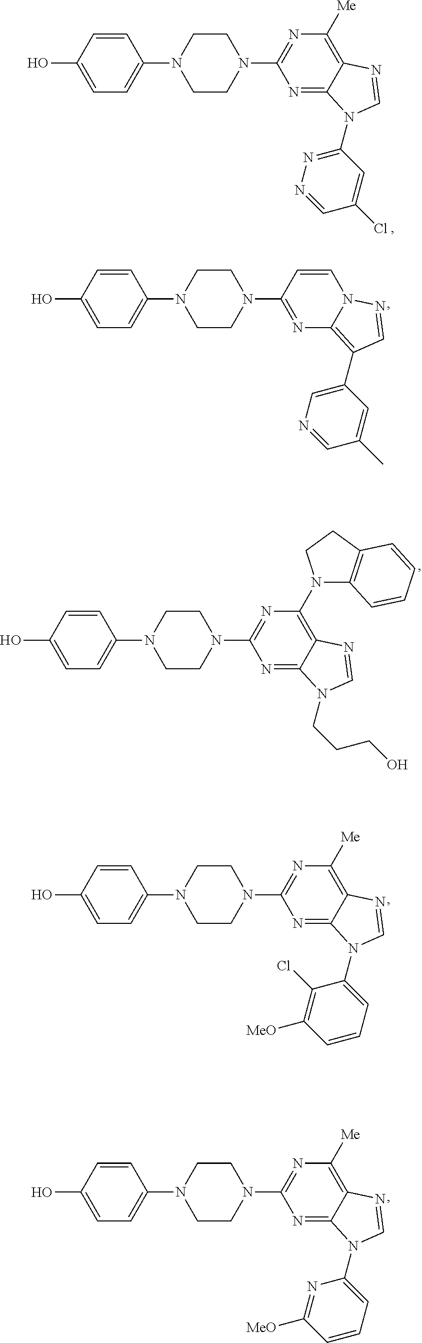 [SMILES: Cc1nc(N2CCN(c3ccc(O)cc3)CC2)nc2c1ncn2-c1cc(Cl)cnn1 ; Cc1cncc(-c2cnn3ccc(N4CCN(c5ccc(O)cc5)CC4)nc23)c1 ; OCCCn1cnc2c(N3CCc4ccccc43)nc(N3CCN(c4ccc(O)cc4)CC3)nc21 ; COc1cccc(-n2cnc3c(C)nc(N4CCN(c5ccc(O)cc5)CC4)nc32)c1Cl ; COc1cccc(-n2cnc3c(C)nc(N4CCN(c5ccc(O)cc5)CC4)nc32)n1]

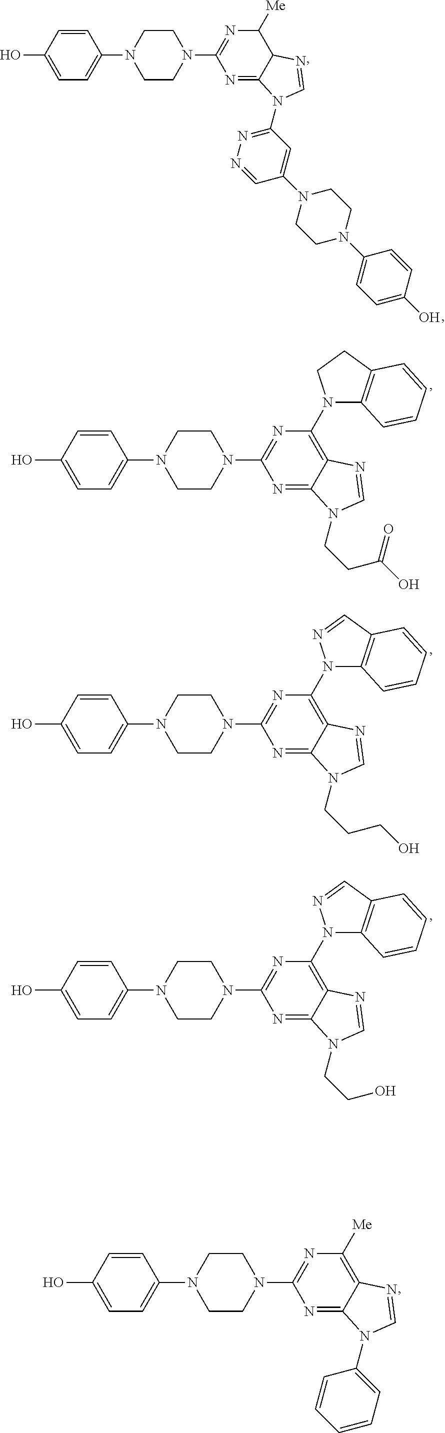

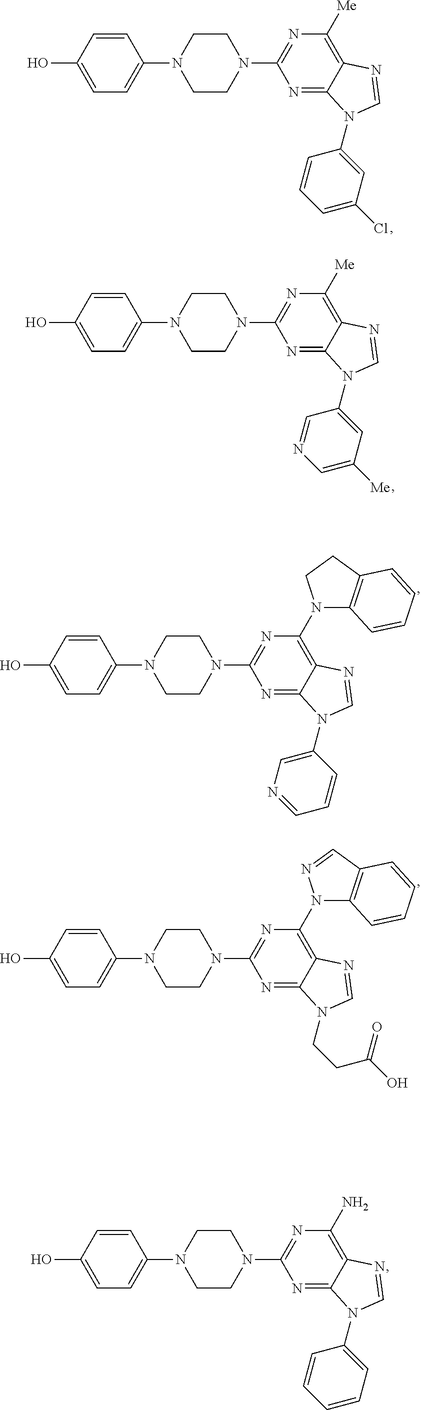

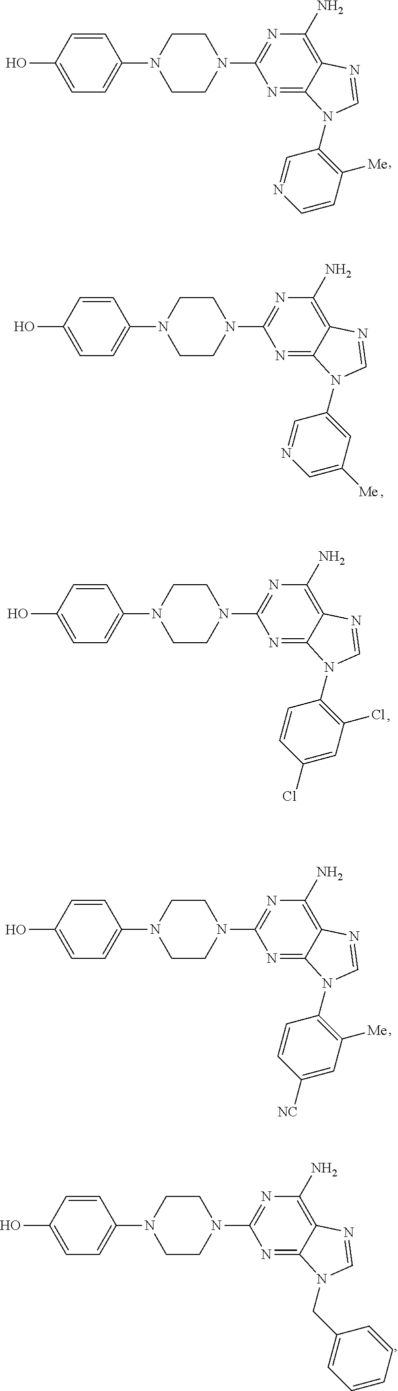 [SMILES: Cc1ccncc1-n1cnc2c(N)nc(N3CCN(c4ccc(O)cc4)CC3)nc21 ; Cc1cncc(-n2cnc3c(N)nc(N4CCN(c5ccc(O)cc5)CC4)nc32)c1 ; Nc1nc(N2CCN(c3ccc(O)cc3)CC2)nc2c1ncn2-c1ccc(Cl)cc1Cl ; Cc1cc(C#N)ccc1-n1cnc2c(N)nc(N3CCN(c4ccc(O)cc4)CC3)nc21 ; Nc1nc(N2CCN(c3ccc(O)cc3)CC2)nc2c1ncn2Cc1ccccc1]

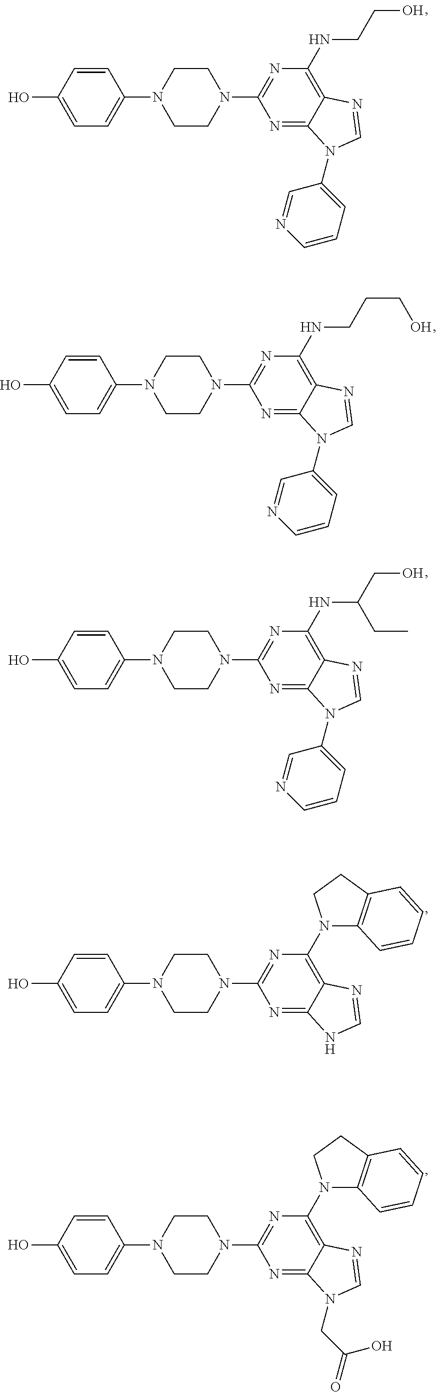 [SMILES: OCCNc1nc(N2CCN(c3ccc(O)cc3)CC2)nc2c1ncn2-c1cccnc1 ; OCCCNc1nc(N2CCN(c3ccc(O)cc3)CC2)nc2c1ncn2-c1cccnc1 ; CCC(CO)Nc1nc(N2CCN(c3ccc(O)cc3)CC2)nc2c1ncn2-c1cccnc1 ; Oc1ccc(N2CCN(c3nc(N4CCc5ccccc54)c4nc[nH]c4n3)CC2)cc1 ; O=C(O)Cn1cnc2c(N3CCc4ccccc43)nc(N3CCN(c4ccc(O)cc4)CC3)nc21]

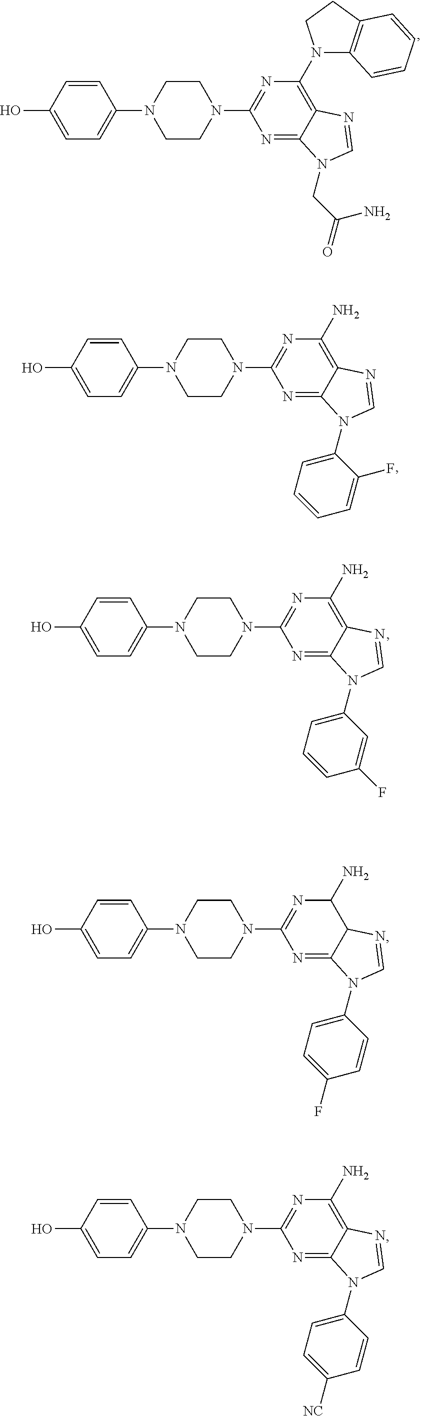 [SMILES: NC(=O)Cn1cnc2c(N3CCc4ccccc43)nc(N3CCN(c4ccc(O)cc4)CC3)nc21 ; Nc1nc(N2CCN(c3ccc(O)cc3)CC2)nc2c1ncn2-c1ccccc1F ; Nc1nc(N2CCN(c3ccc(O)cc3)CC2)nc2c1ncn2-c1cccc(F)c1 ; NC1N=C(N2CCN(c3ccc(O)cc3)CC2)N=C2C1N=CN2c1ccc(F)cc1 ; N#Cc1ccc(-n2cnc3c(N)nc(N4CCN(c5ccc(O)cc5)CC4)nc32)cc1]

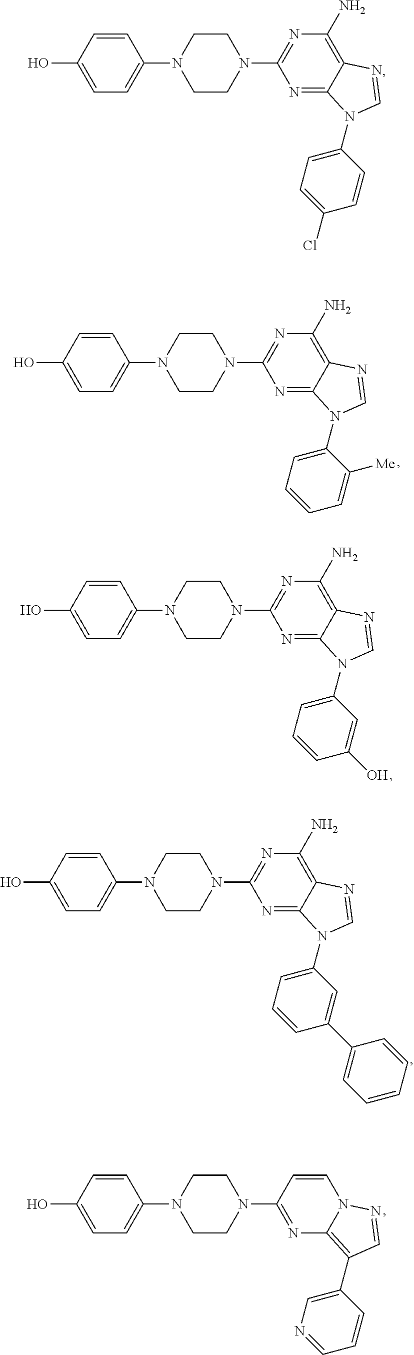 [SMILES: Nc1nc(N2CCN(c3ccc(O)cc3)CC2)nc2c1ncn2-c1ccc(Cl)cc1 ; Cc1ccccc1-n1cnc2c(N)nc(N3CCN(c4ccc(O)cc4)CC3)nc21 ; Nc1nc(N2CCN(c3ccc(O)cc3)CC2)nc2c1ncn2-c1cccc(O)c1 ; Nc1nc(N2CCN(c3ccc(O)cc3)CC2)nc2c1ncn2-c1cccc(-c2ccccc2)c1 ; Oc1ccc(N2CCN(c3ccn4ncc(-c5cccnc5)c4n3)CC2)cc1]

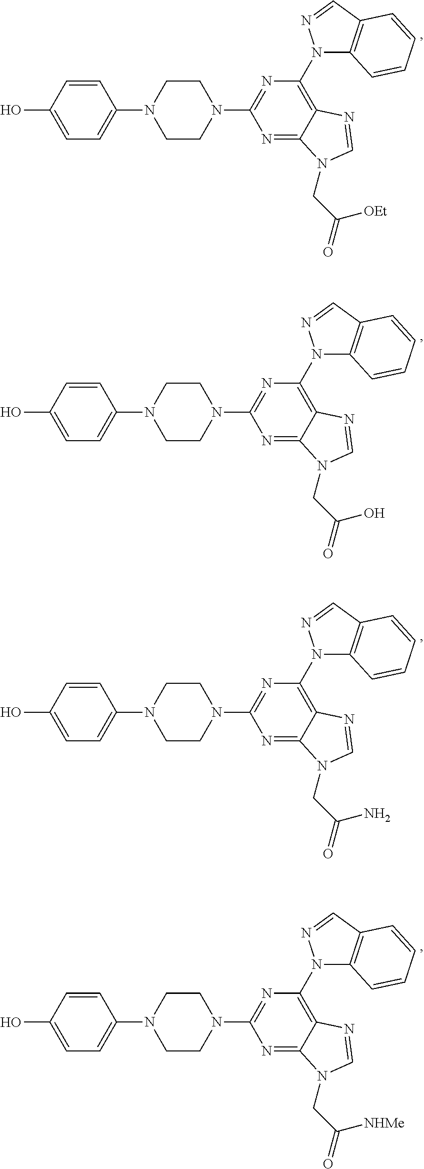 [SMILES: CCOC(=O)Cn1cnc2c(-n3ncc4ccccc43)nc(N3CCN(c4ccc(O)cc4)CC3)nc21 ; O=C(O)Cn1cnc2c(-n3ncc4ccccc43)nc(N3CCN(c4ccc(O)cc4)CC3)nc21 ; NC(=O)Cn1cnc2c(-n3ncc4ccccc43)nc(N3CCN(c4ccc(O)cc4)CC3)nc21 ; CNC(=O)Cn1cnc2c(-n3ncc4ccccc43)nc(N3CCN(c4ccc(O)cc4)CC3)nc21]

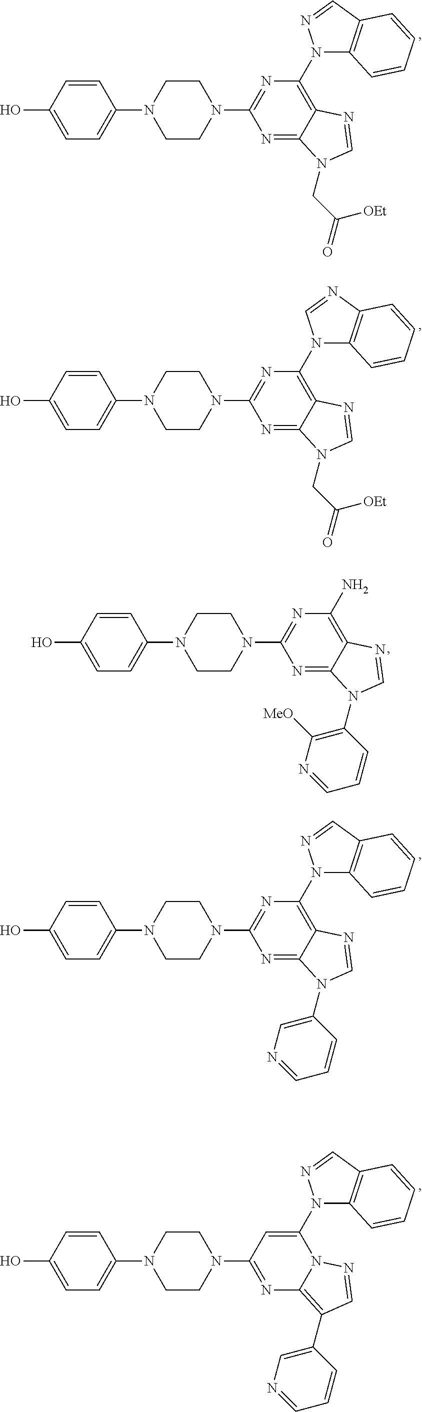

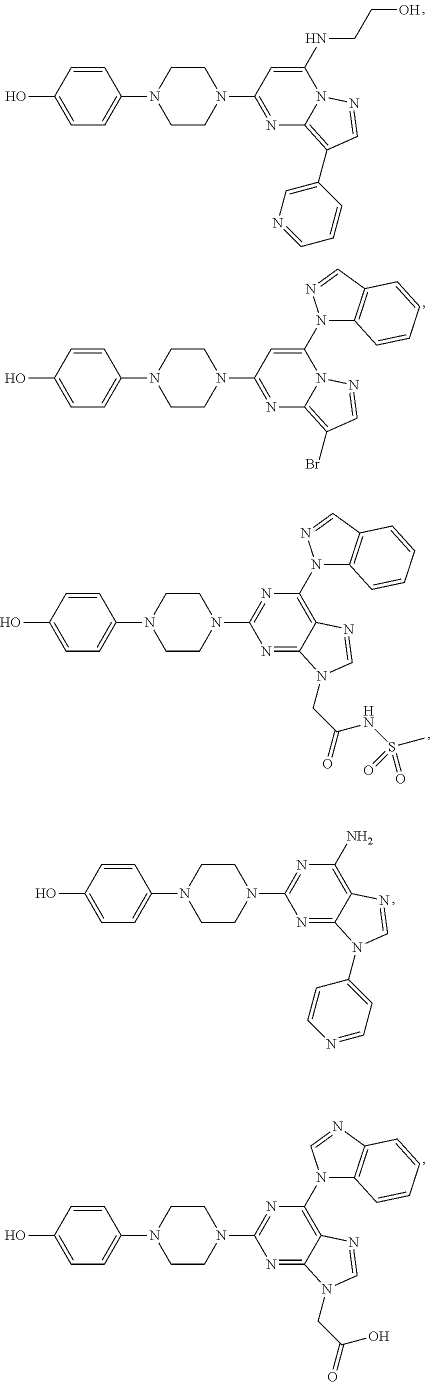

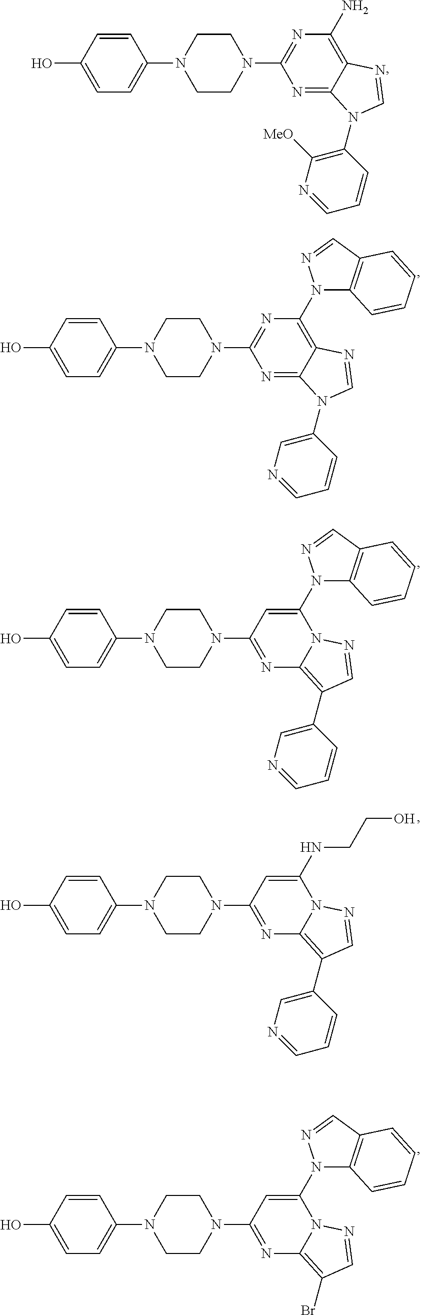

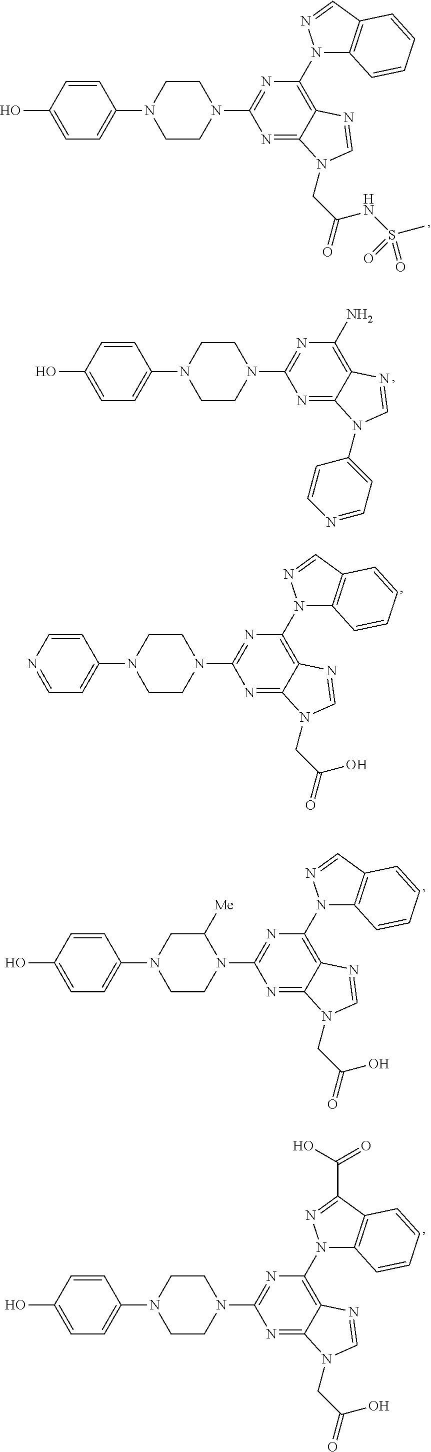

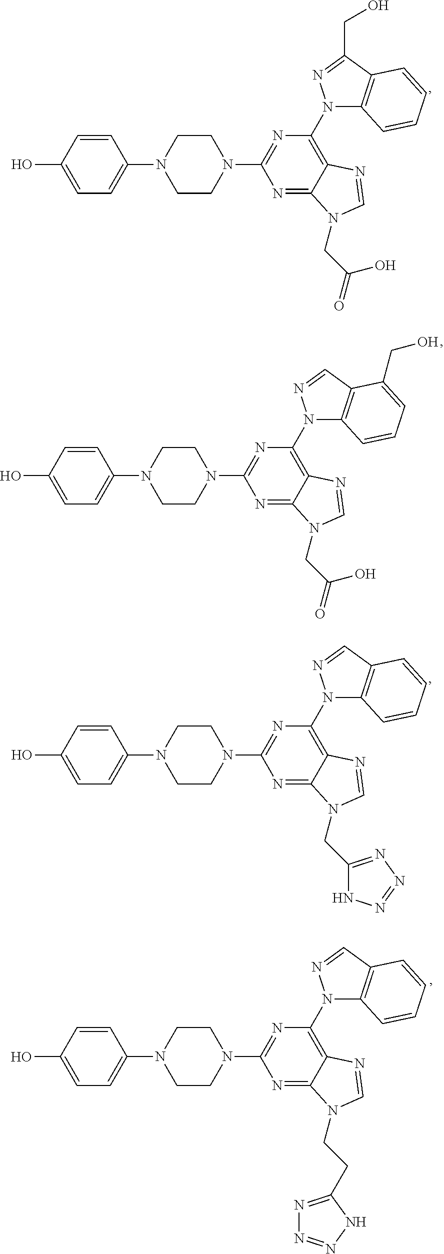

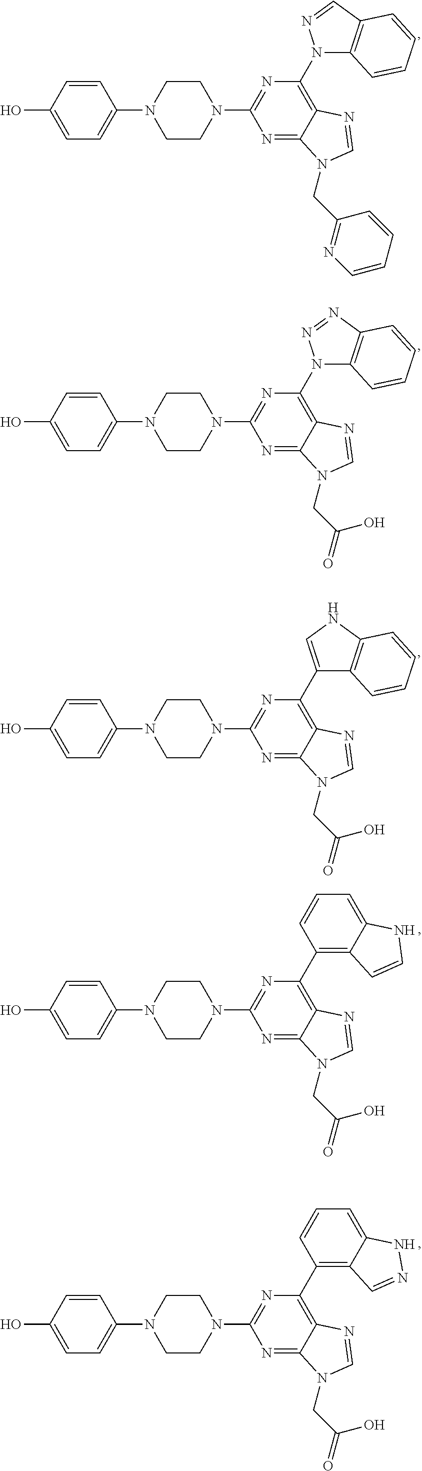

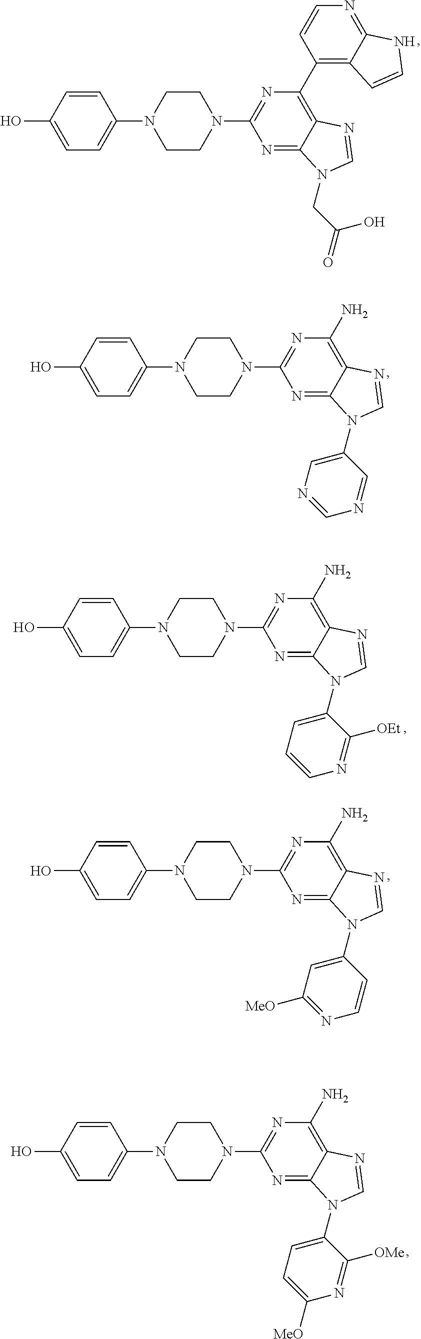

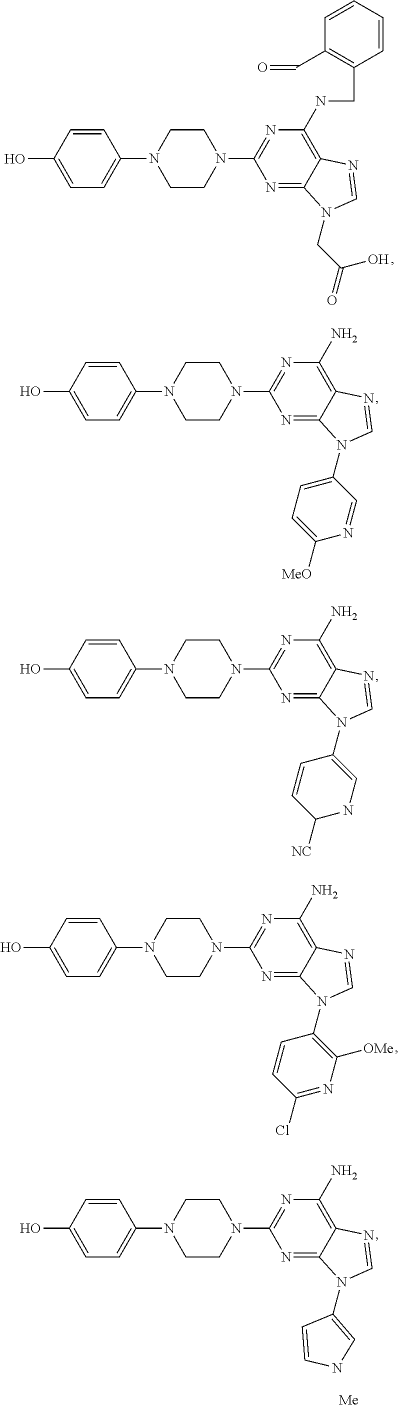

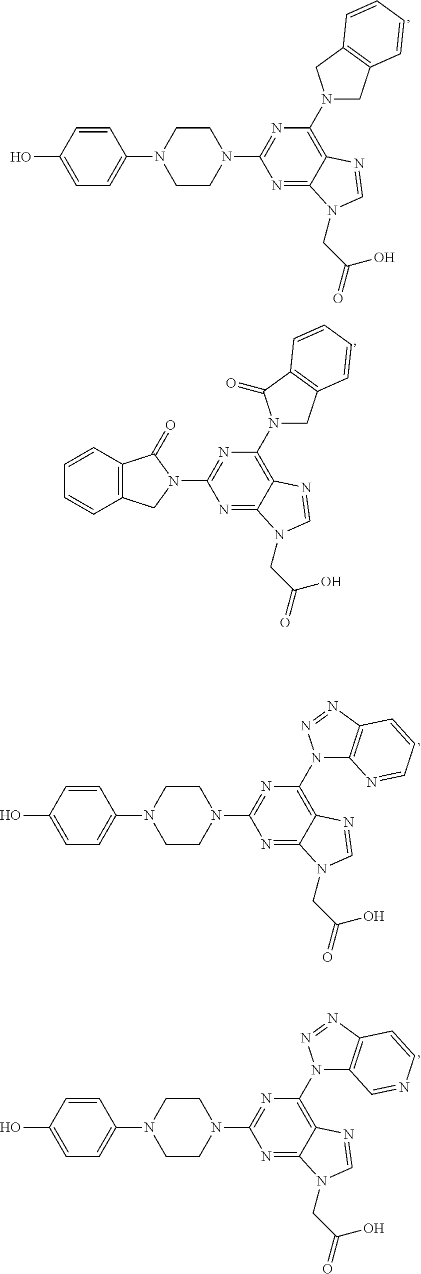

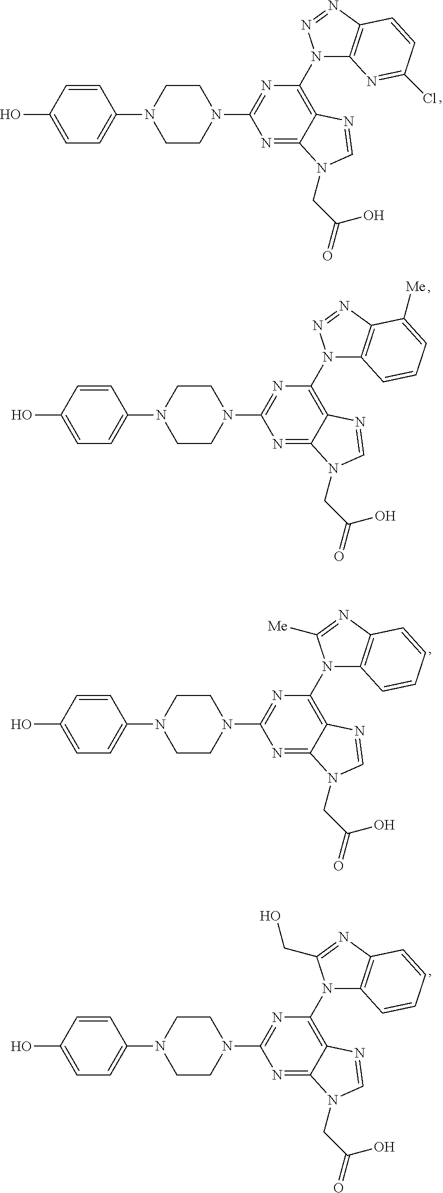 [SMILES: O=C(O)Cn1cnc2c(-n3nnc4ccc(Cl)nc43)nc(N3CCN(c4ccc(O)cc4)CC3)nc21 ; Cc1cccc2c1nnn2-c1nc(N2CCN(c3ccc(O)cc3)CC2)nc2c1ncn2CC(=O)O ; Cc1nc2ccccc2n1-c1nc(N2CCN(c3ccc(O)cc3)CC2)nc2c1ncn2CC(=O)O ; O=C(O)Cn1cnc2c(-n3c(CO)nc4ccccc43)nc(N3CCN(c4ccc(O)cc4)CC3)nc21]

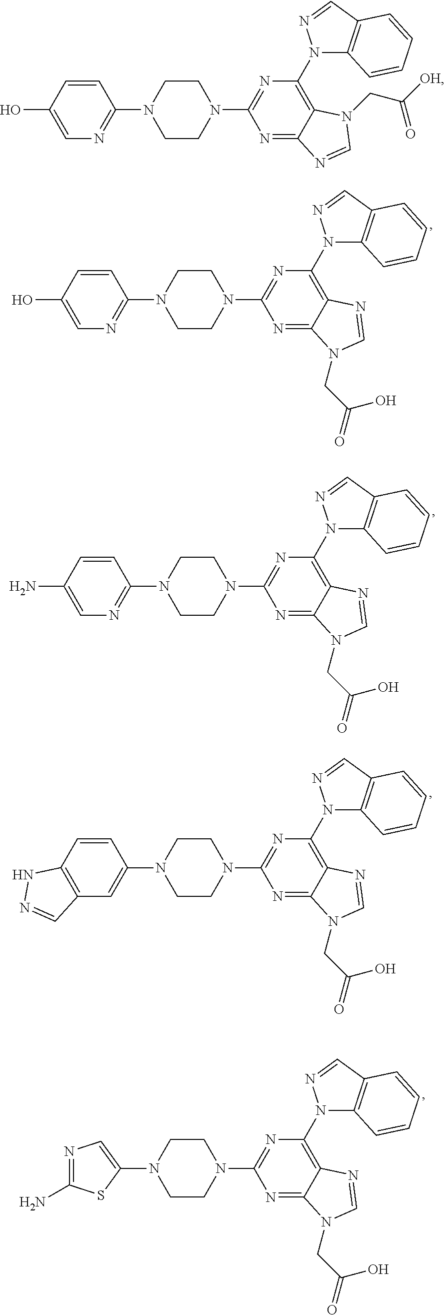

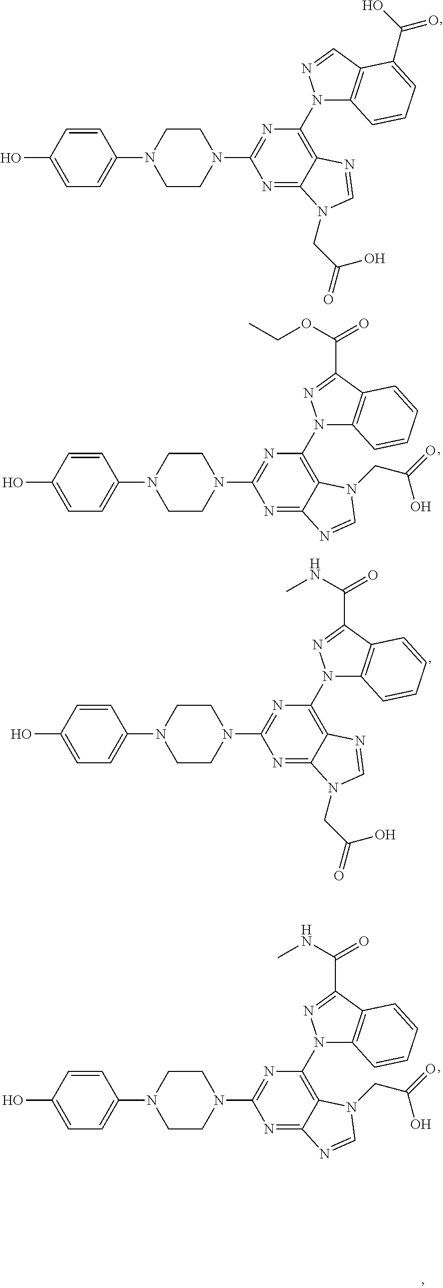

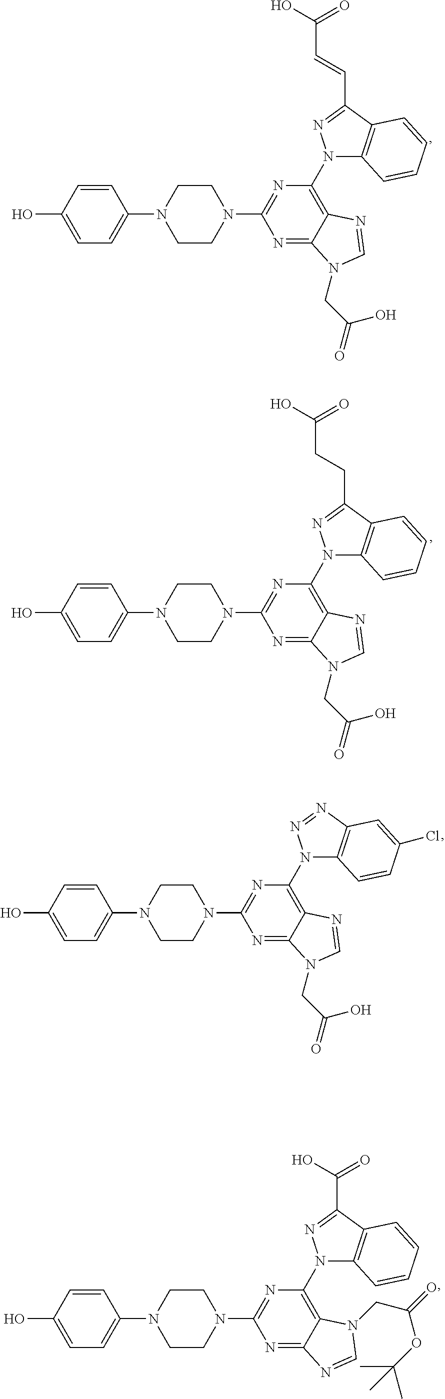 [SMILES: O=C(O)/C=C/c1nn(-c2nc(N3CCN(c4ccc(O)cc4)CC3)nc3c2ncn3CC(=O)O)c2ccccc12 ; O=C(O)CCc1nn(-c2nc(N3CCN(c4ccc(O)cc4)CC3)nc3c2ncn3CC(=O)O)c2ccccc12 ; O=C(O)Cn1cnc2c(-n3nnc4cc(Cl)ccc43)nc(N3CCN(c4ccc(O)cc4)CC3)nc21 ; CC(C)(C)OC(=O)Cn1cnc2nc(N3CCN(c4ccc(O)cc4)CC3)nc(-n3nc(C(=O)O)c4ccccc43)c21]

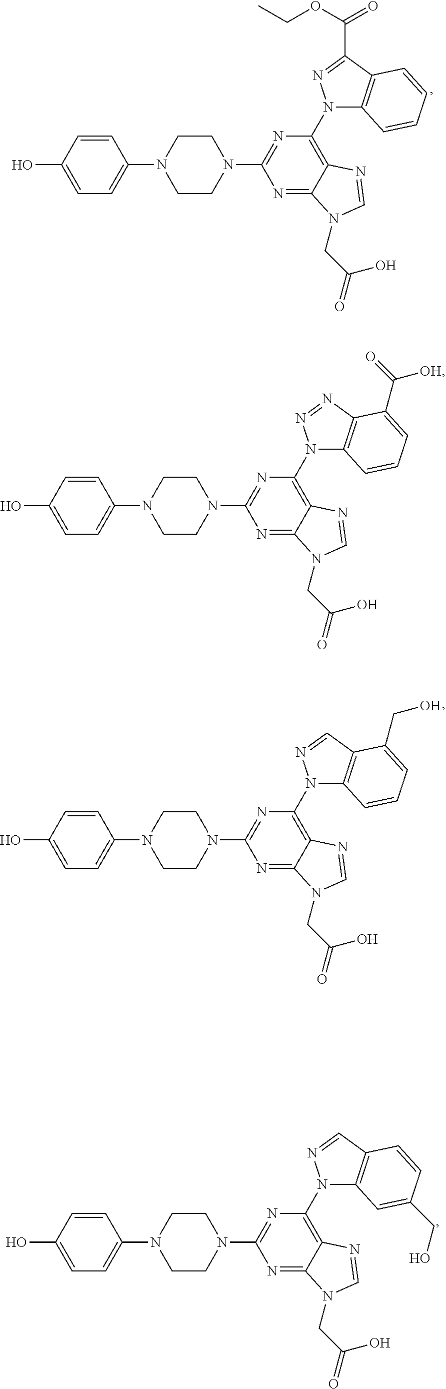 [SMILES: CCOC(=O)c1nn(-c2nc(N3CCN(c4ccc(O)cc4)CC3)nc3c2ncn3CC(=O)O)c2ccccc12 ; O=C(O)Cn1cnc2c(-n3nnc4c(C(=O)O)cccc43)nc(N3CCN(c4ccc(O)cc4)CC3)nc21 ; O=C(O)Cn1cnc2c(-n3ncc4c(CO)cccc43)nc(N3CCN(c4ccc(O)cc4)CC3)nc21 ; O=C(O)Cn1cnc2c(-n3ncc4ccc(CO)cc43)nc(N3CCN(c4ccc(O)cc4)CC3)nc21]

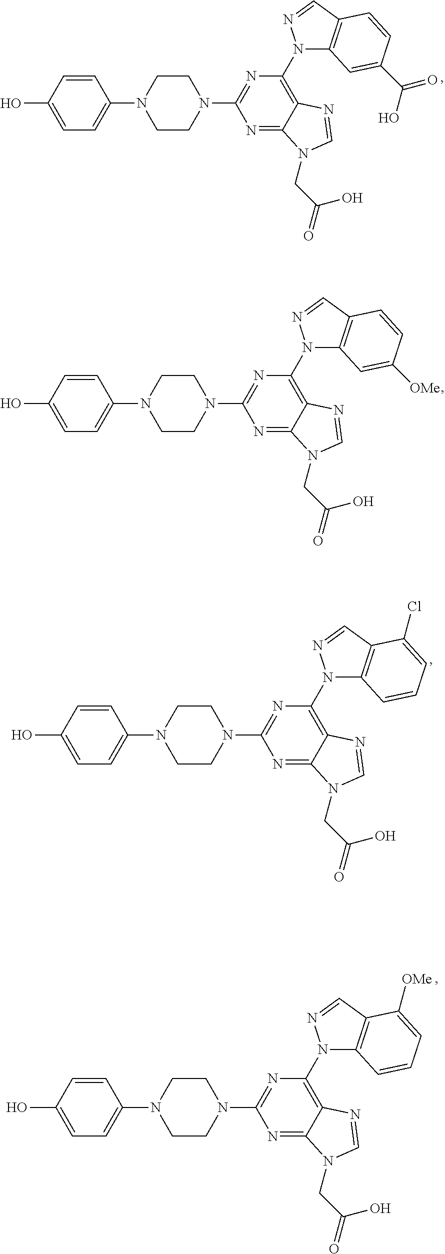 [SMILES: O=C(O)Cn1cnc2c(-n3ncc4ccc(C(=O)O)cc43)nc(N3CCN(c4ccc(O)cc4)CC3)nc21 ; COc1ccc2cnn(-c3nc(N4CCN(c5ccc(O)cc5)CC4)nc4c3ncn4CC(=O)O)c2c1 ; O=C(O)Cn1cnc2c(-n3ncc4c(Cl)cccc43)nc(N3CCN(c4ccc(O)cc4)CC3)nc21 ; COc1cccc2c1cnn2-c1nc(N2CCN(c3ccc(O)cc3)CC2)nc2c1ncn2CC(=O)O]

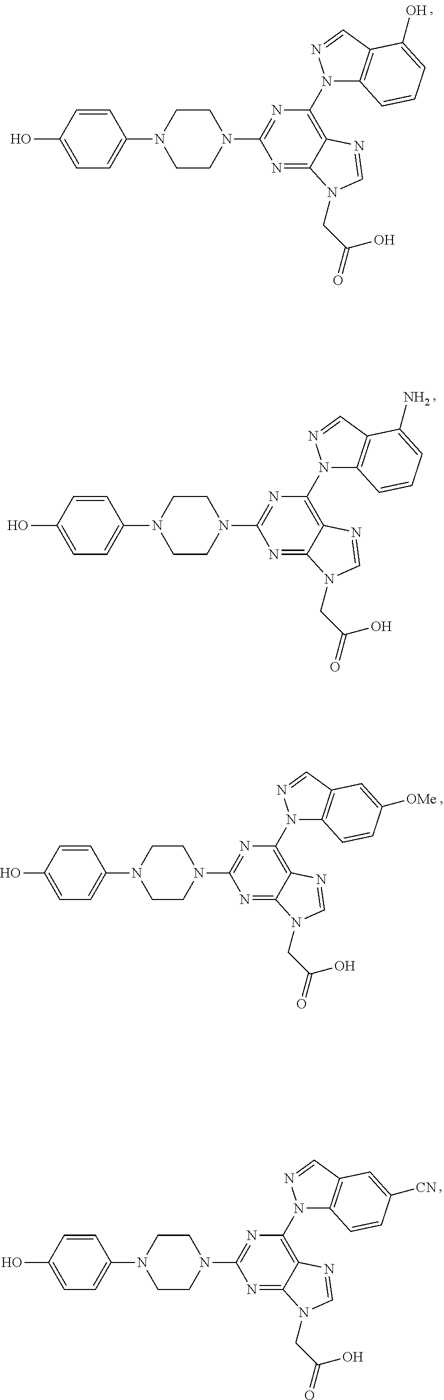 [SMILES: O=C(O)Cn1cnc2c(-n3ncc4c(O)cccc43)nc(N3CCN(c4ccc(O)cc4)CC3)nc21 ; Nc1cccc2c1cnn2-c1nc(N2CCN(c3ccc(O)cc3)CC2)nc2c1ncn2CC(=O)O ; COc1ccc2c(cnn2-c2nc(N3CCN(c4ccc(O)cc4)CC3)nc3c2ncn3CC(=O)O)c1 ; N#Cc1ccc2c(cnn2-c2nc(N3CCN(c4ccc(O)cc4)CC3)nc3c2ncn3CC(=O)O)c1]

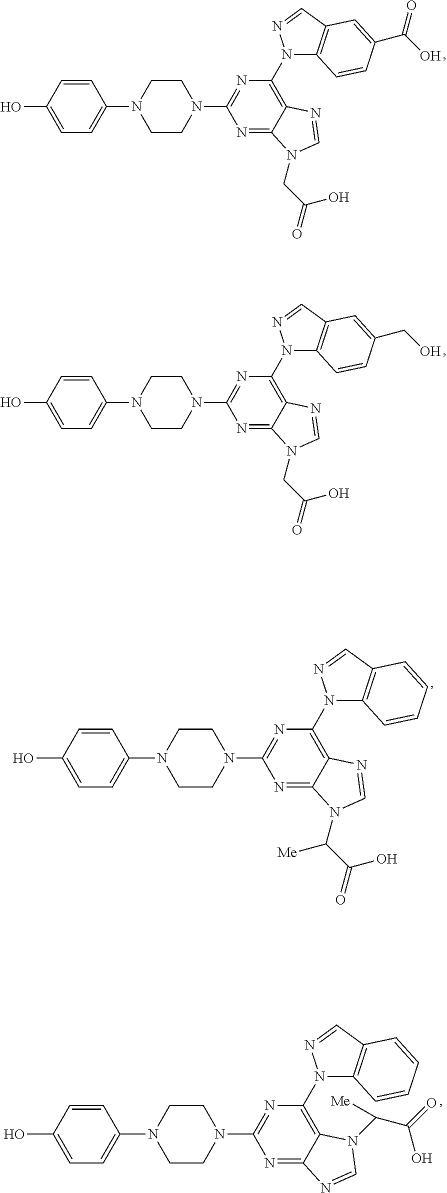 [SMILES: O=C(O)Cn1cnc2c(-n3ncc4cc(C(=O)O)ccc43)nc(N3CCN(c4ccc(O)cc4)CC3)nc21 ; O=C(O)Cn1cnc2c(-n3ncc4cc(CO)ccc43)nc(N3CCN(c4ccc(O)cc4)CC3)nc21 ; CC(C(=O)O)n1cnc2c(-n3ncc4ccccc43)nc(N3CCN(c4ccc(O)cc4)CC3)nc21 ; CC(C(=O)O)n1cnc2nc(N3CCN(c4ccc(O)cc4)CC3)nc(-n3ncc4ccccc43)c21]

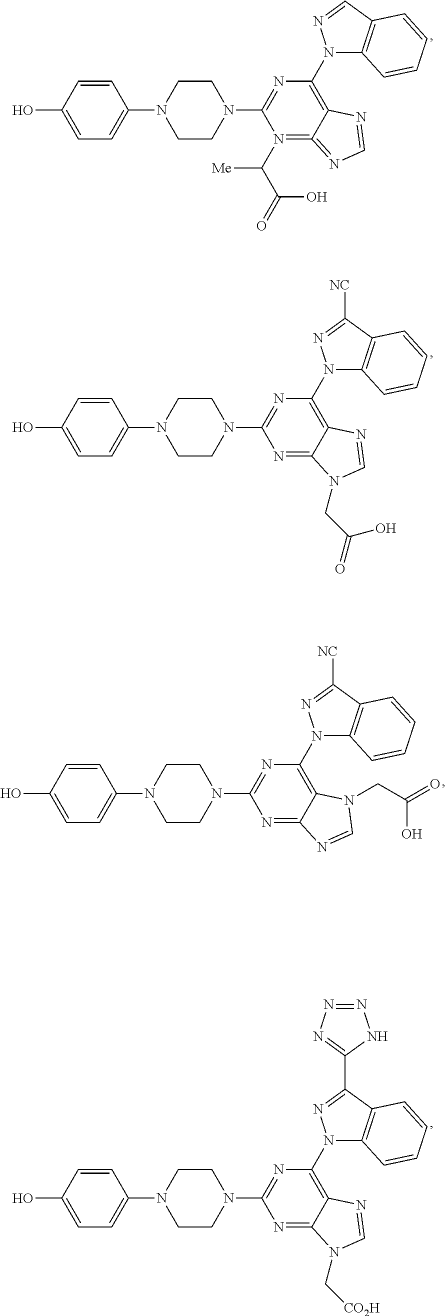

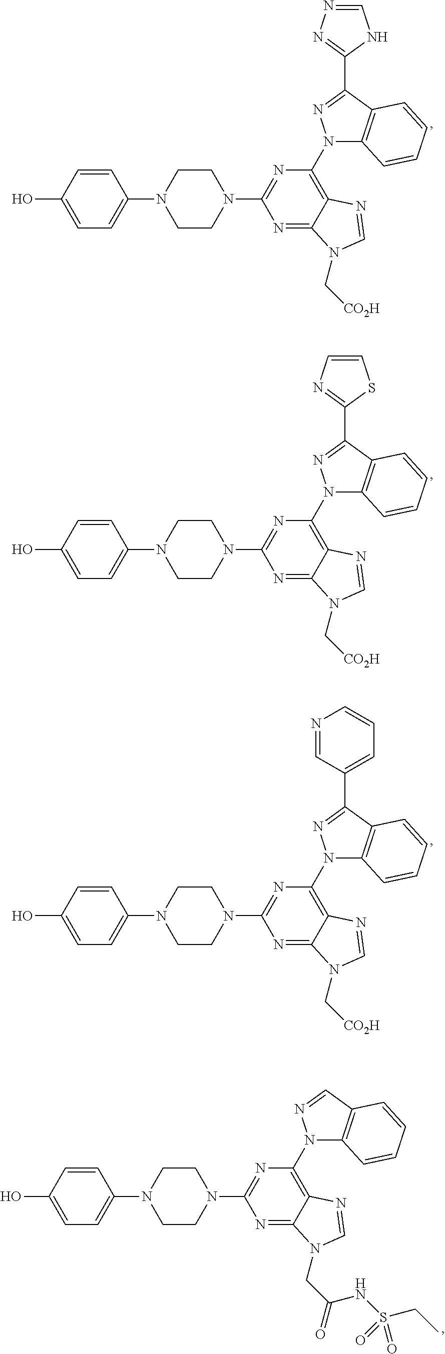 [SMILES: O=C(O)Cn1cnc2c(-n3nc(-c4nnc[nH]4)c4ccccc43)nc(N3CCN(c4ccc(O)cc4)CC3)nc21 ; O=C(O)Cn1cnc2c(-n3nc(-c4nccs4)c4ccccc43)nc(N3CCN(c4ccc(O)cc4)CC3)nc21 ; O=C(O)Cn1cnc2c(-n3nc(-c4cccnc4)c4ccccc43)nc(N3CCN(c4ccc(O)cc4)CC3)nc21 ; CCS(=O)(=O)NC(=O)Cn1cnc2c(-n3ncc4ccccc43)nc(N3CCN(c4ccc(O)cc4)CC3)nc21]

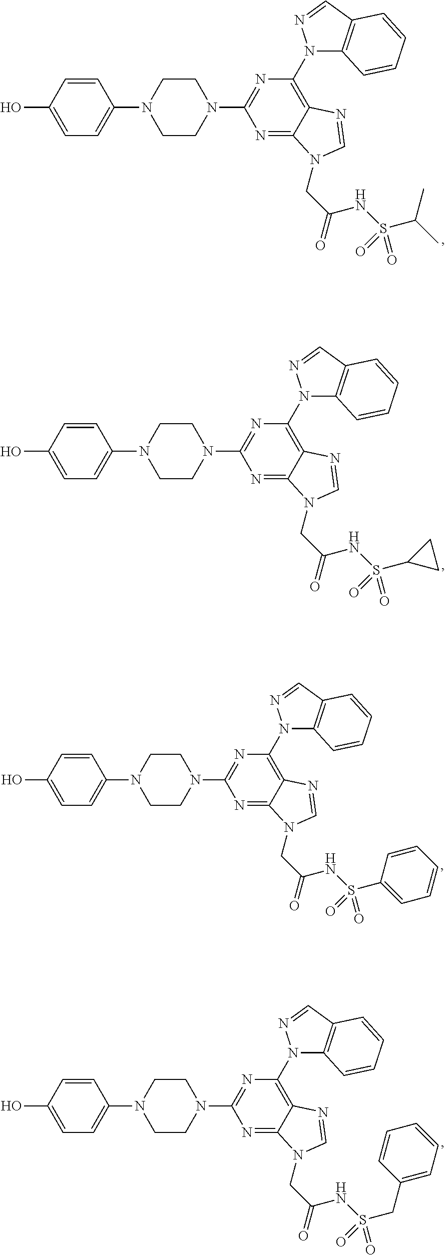 [SMILES: CC(C)S(=O)(=O)NC(=O)Cn1cnc2c(-n3ncc4ccccc43)nc(N3CCN(c4ccc(O)cc4)CC3)nc21 ; O=C(Cn1cnc2c(-n3ncc4ccccc43)nc(N3CCN(c4ccc(O)cc4)CC3)nc21)NS(=O)(=O)C1CC1 ; O=C(Cn1cnc2c(-n3ncc4ccccc43)nc(N3CCN(c4ccc(O)cc4)CC3)nc21)NS(=O)(=O)c1ccccc1 ; O=C(Cn1cnc2c(-n3ncc4ccccc43)nc(N3CCN(c4ccc(O)cc4)CC3)nc21)NS(=O)(=O)Cc1ccccc1]

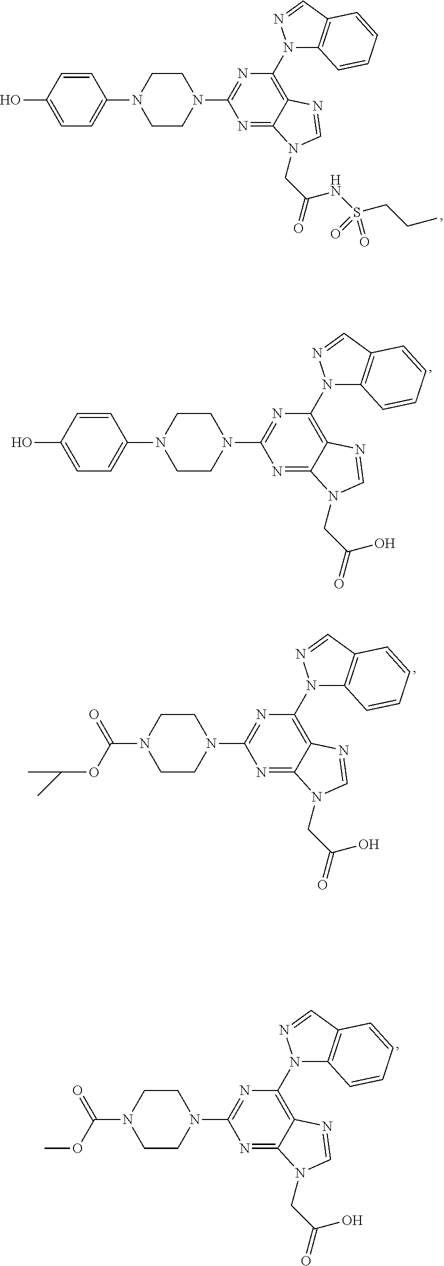

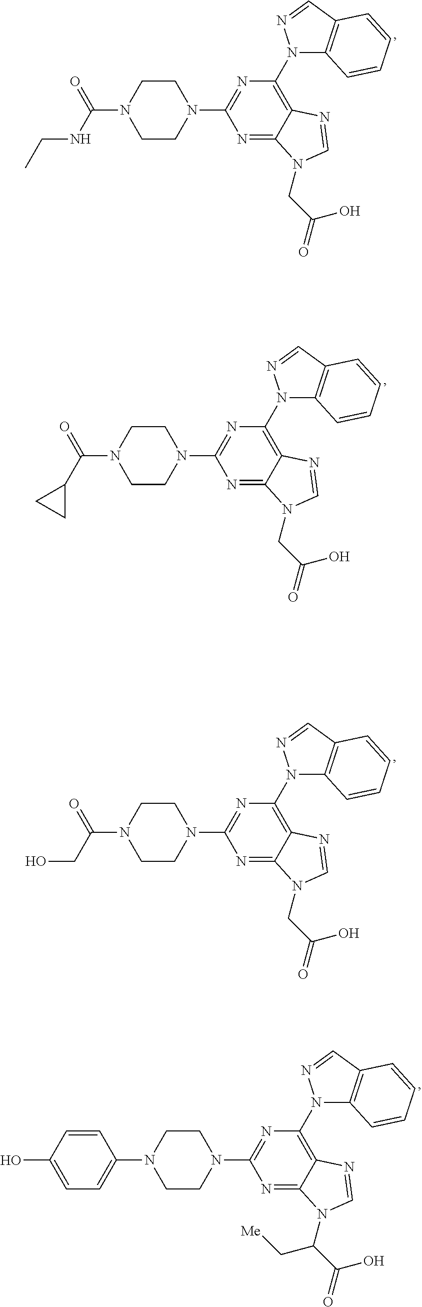 [SMILES: CCNC(=O)N1CCN(c2nc(-n3ncc4ccccc43)c3ncn(CC(=O)O)c3n2)CC1 ; O=C(O)Cn1cnc2c(-n3ncc4ccccc43)nc(N3CCN(C(=O)C4CC4)CC3)nc21 ; O=C(O)Cn1cnc2c(-n3ncc4ccccc43)nc(N3CCN(C(=O)CO)CC3)nc21 ; CCC(C(=O)O)n1cnc2c(-n3ncc4ccccc43)nc(N3CCN(c4ccc(O)cc4)CC3)nc21]

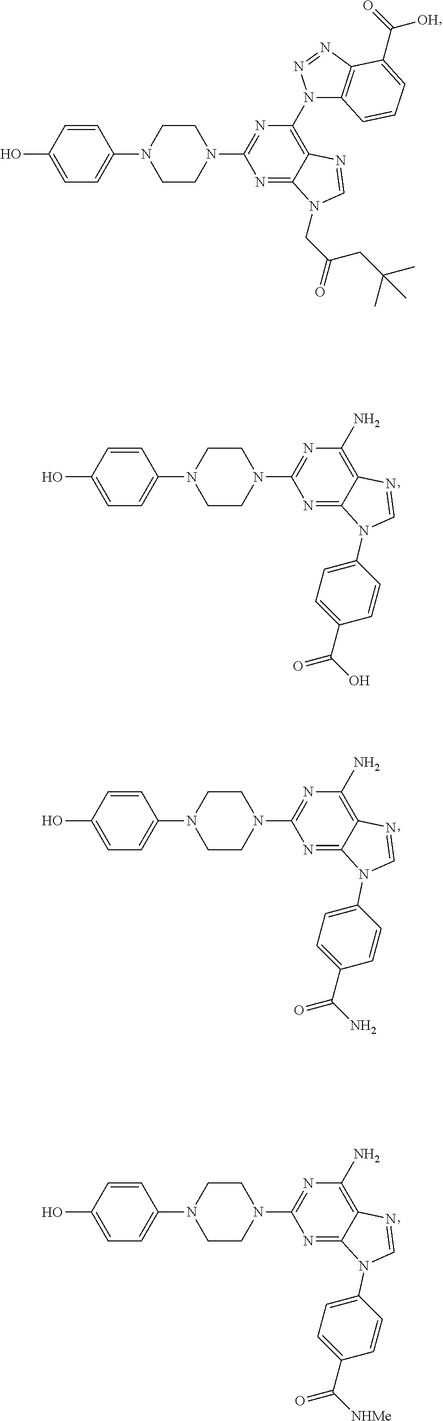 [SMILES: CC(C)(C)CC(=O)Cn1cnc2c(-n3nnc4c(C(=O)O)cccc43)nc(N3CCN(c4ccc(O)cc4)CC3)nc21 ; Nc1nc(N2CCN(c3ccc(O)cc3)CC2)nc2c1ncn2-c1ccc(C(=O)O)cc1 ; NC(=O)c1ccc(-n2cnc3c(N)nc(N4CCN(c5ccc(O)cc5)CC4)nc32)cc1 ; CNC(=O)c1ccc(-n2cnc3c(N)nc(N4CCN(c5ccc(O)cc5)CC4)nc32)cc1]

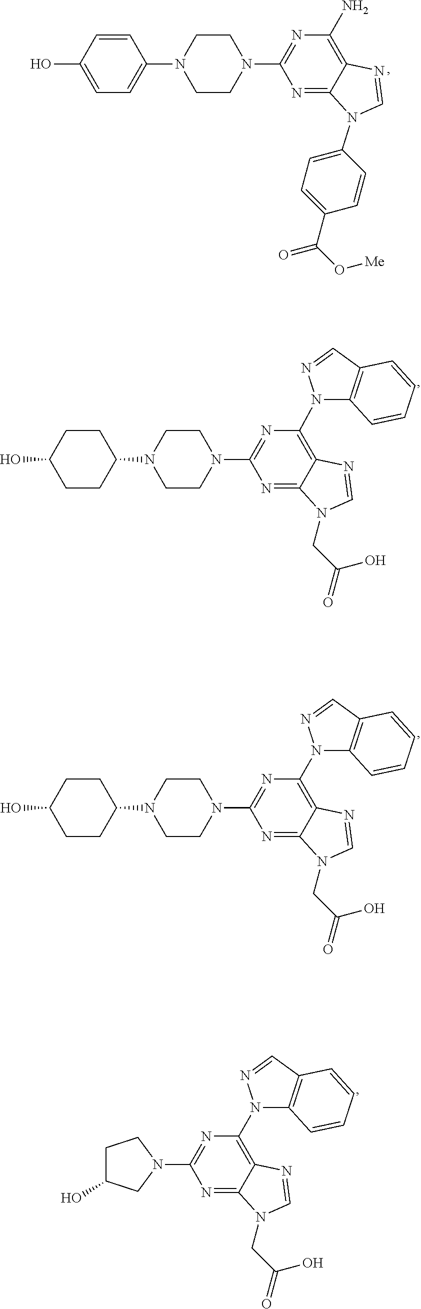 [SMILES: COC(=O)c1ccc(-n2cnc3c(N)nc(N4CCN(c5ccc(O)cc5)CC4)nc32)cc1 ; O=C(O)Cn1cnc2c(-n3ncc4ccccc43)nc(N3CCN([C@H]4CC[C@@H](O)CC4)CC3)nc21 ; O=C(O)Cn1cnc2c(-n3ncc4ccccc43)nc(N3CCN([C@H]4CC[C@@H](O)CC4)CC3)nc21 ; O=C(O)Cn1cnc2c(-n3ncc4ccccc43)nc(N3CC[C@@H](O)C3)nc21]

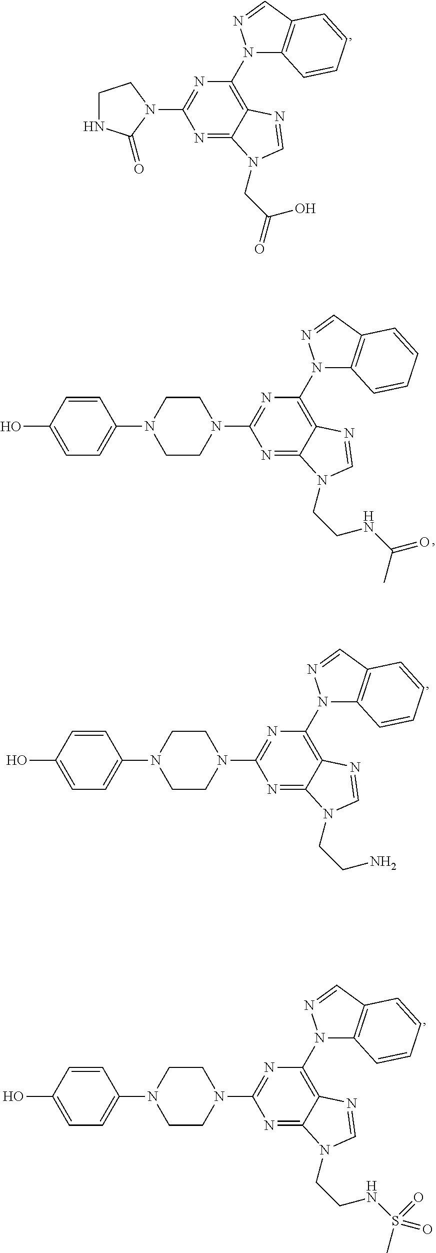 [SMILES: O=C(O)Cn1cnc2c(-n3ncc4ccccc43)nc(N3CCNC3=O)nc21 ; CC(=O)NCCn1cnc2c(-n3ncc4ccccc43)nc(N3CCN(c4ccc(O)cc4)CC3)nc21 ; NCCn1cnc2c(-n3ncc4ccccc43)nc(N3CCN(c4ccc(O)cc4)CC3)nc21 ; CS(=O)(=O)NCCn1cnc2c(-n3ncc4ccccc43)nc(N3CCN(c4ccc(O)cc4)CC3)nc21]

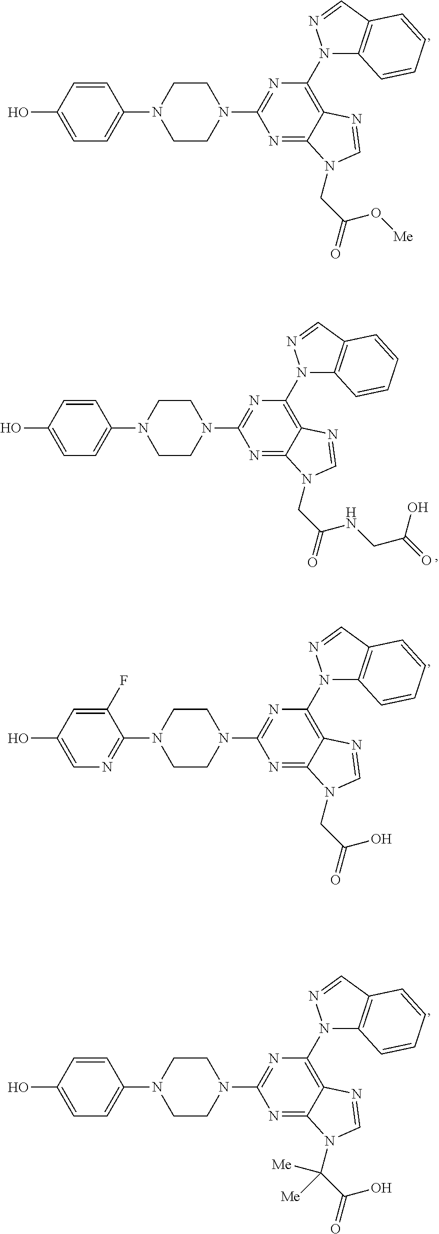 [SMILES: COC(=O)Cn1cnc2c(-n3ncc4ccccc43)nc(N3CCN(c4ccc(O)cc4)CC3)nc21 ; O=C(O)CNC(=O)Cn1cnc2c(-n3ncc4ccccc43)nc(N3CCN(c4ccc(O)cc4)CC3)nc21 ; O=C(O)Cn1cnc2c(-n3ncc4ccccc43)nc(N3CCN(c4ncc(O)cc4F)CC3)nc21 ; CC(C)(C(=O)O)n1cnc2c(-n3ncc4ccccc43)nc(N3CCN(c4ccc(O)cc4)CC3)nc21]

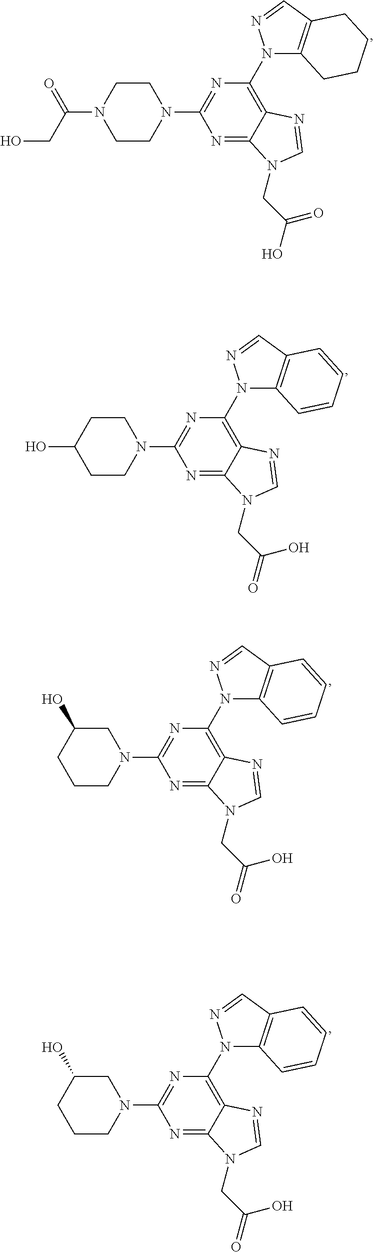 [SMILES: O=C(O)Cn1cnc2c(-n3ncc4c3CCCC4)nc(N3CCN(C(=O)CO)CC3)nc21 ; O=C(O)Cn1cnc2c(-n3ncc4ccccc43)nc(N3CCC(O)CC3)nc21 ; O=C(O)Cn1cnc2c(-n3ncc4ccccc43)nc(N3CCC[C@@H](O)C3)nc21 ; O=C(O)Cn1cnc2c(-n3ncc4ccccc43)nc(N3CCC[C@H](O)C3)nc21]

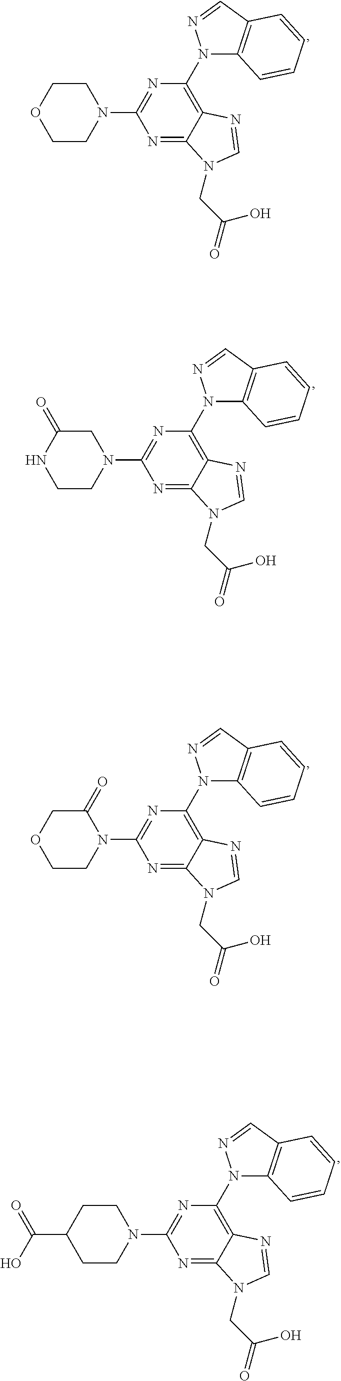 [SMILES: O=C(O)Cn1cnc2c(-n3ncc4ccccc43)nc(N3CCOCC3)nc21 ; O=C(O)Cn1cnc2c(-n3ncc4ccccc43)nc(N3CCNC(=O)C3)nc21 ; O=C(O)Cn1cnc2c(-n3ncc4ccccc43)nc(N3CCOCC3=O)nc21 ; O=C(O)Cn1cnc2c(-n3ncc4ccccc43)nc(N3CCC(C(=O)O)CC3)nc21]

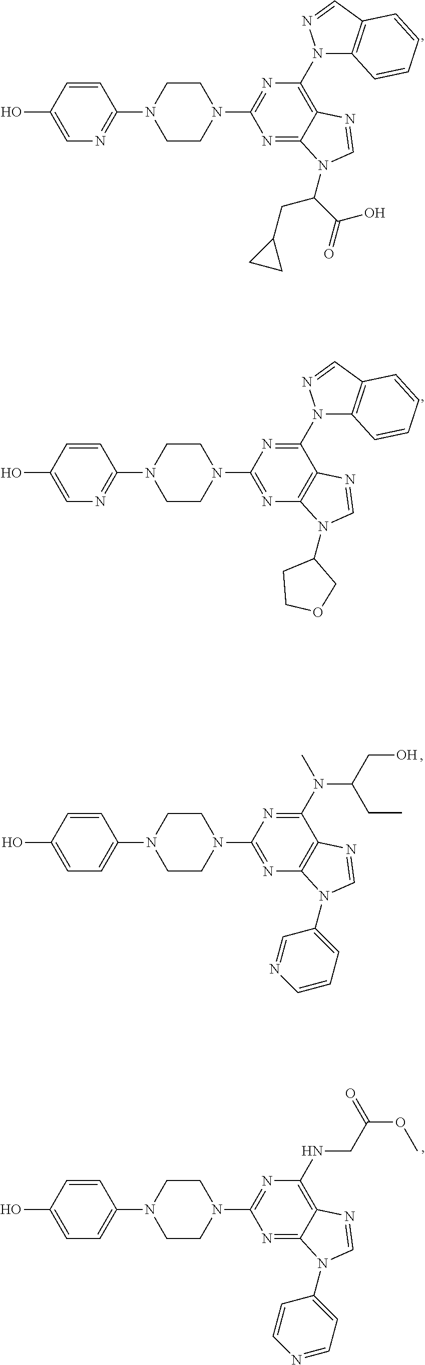 [SMILES: O=C(O)C(CC1CC1)n1cnc2c(-n3ncc4ccccc43)nc(N3CCN(c4ccc(O)cn4)CC3)nc21 ; Oc1ccc(N2CCN(c3nc(-n4ncc5ccccc54)c4ncn(C5CCOC5)c4n3)CC2)nc1 ; CCC(CO)N(C)c1nc(N2CCN(c3ccc(O)cc3)CC2)nc2c1ncn2-c1cccnc1 ; COC(=O)CNc1nc(N2CCN(c3ccc(O)cc3)CC2)nc2c1ncn2-c1ccncc1]

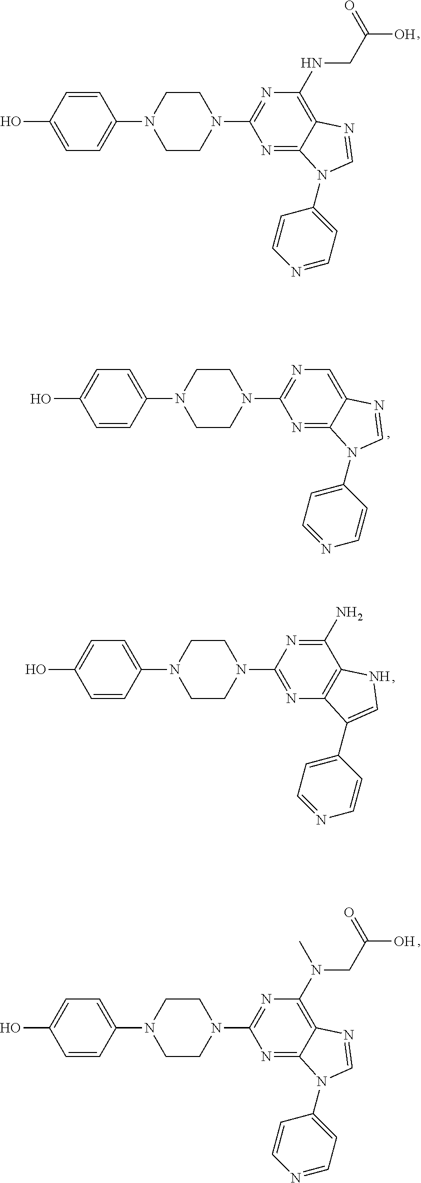 [SMILES: O=C(O)CNc1nc(N2CCN(c3ccc(O)cc3)CC2)nc2c1ncn2-c1ccncc1 ; Oc1ccc(N2CCN(c3ncc4ncn(-c5ccncc5)c4n3)CC2)cc1 ; Nc1nc(N2CCN(c3ccc(O)cc3)CC2)nc2c(-c3ccncc3)c[nH]c12 ; CN(CC(=O)O)c1nc(N2CCN(c3ccc(O)cc3)CC2)nc2c1ncn2-c1ccncc1]

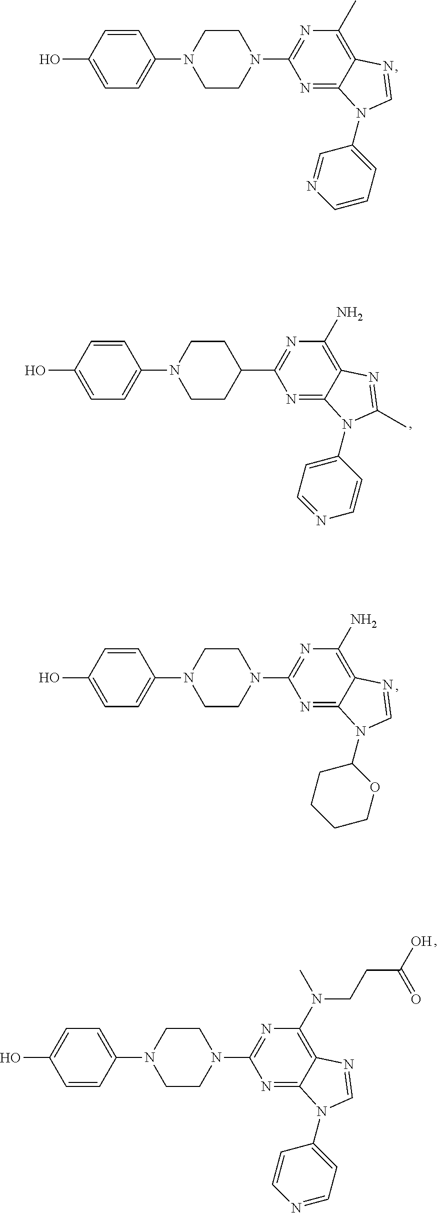 [SMILES: Cc1nc(N2CCN(c3ccc(O)cc3)CC2)nc2c1ncn2-c1cccnc1 ; Cc1nc2c(N)nc(C3CCN(c4ccc(O)cc4)CC3)nc2n1-c1ccncc1 ; Nc1nc(N2CCN(c3ccc(O)cc3)CC2)nc2c1ncn2C1CCCCO1 ; CN(CCC(=O)O)c1nc(N2CCN(c3ccc(O)cc3)CC2)nc2c1ncn2-c1ccncc1]

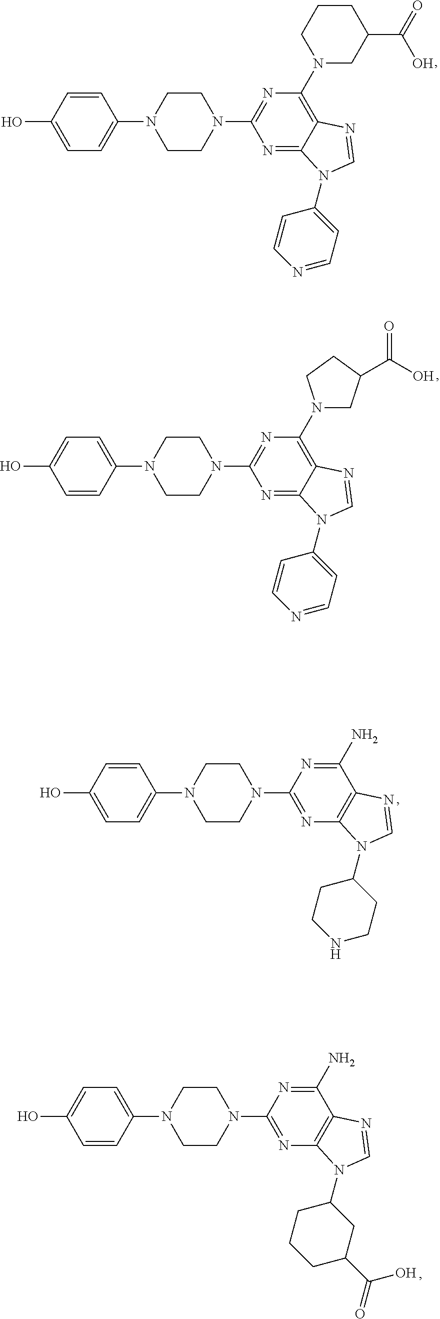 [SMILES: O=C(O)C1CCCN(c2nc(N3CCN(c4ccc(O)cc4)CC3)nc3c2ncn3-c2ccncc2)C1 ; O=C(O)C1CCN(c2nc(N3CCN(c4ccc(O)cc4)CC3)nc3c2ncn3-c2ccncc2)C1 ; Nc1nc(N2CCN(c3ccc(O)cc3)CC2)nc2c1ncn2C1CCNCC1 ; Nc1nc(N2CCN(c3ccc(O)cc3)CC2)nc2c1ncn2C1CCCC(C(=O)O)C1]

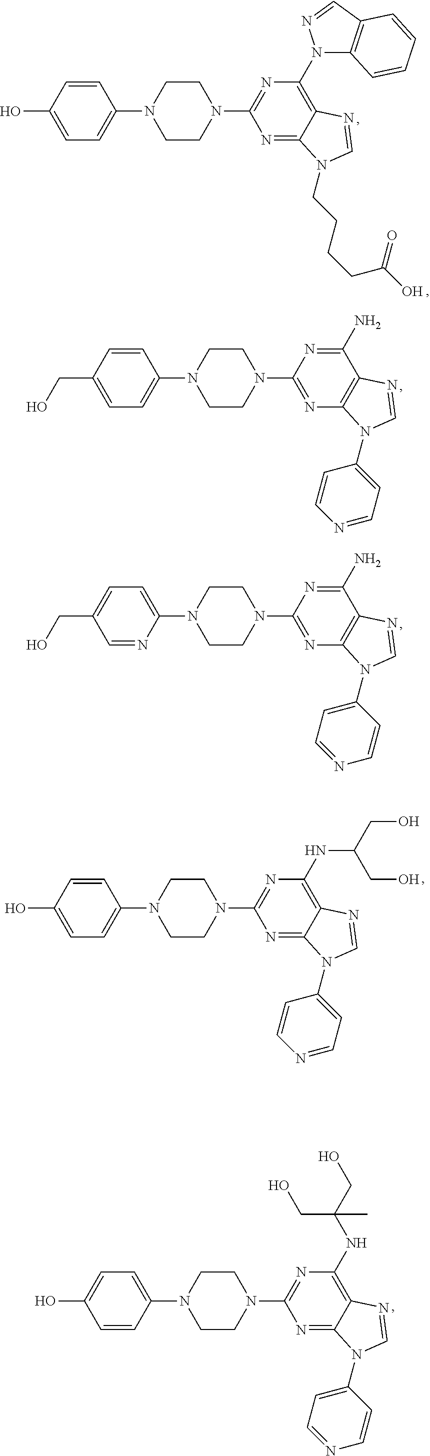

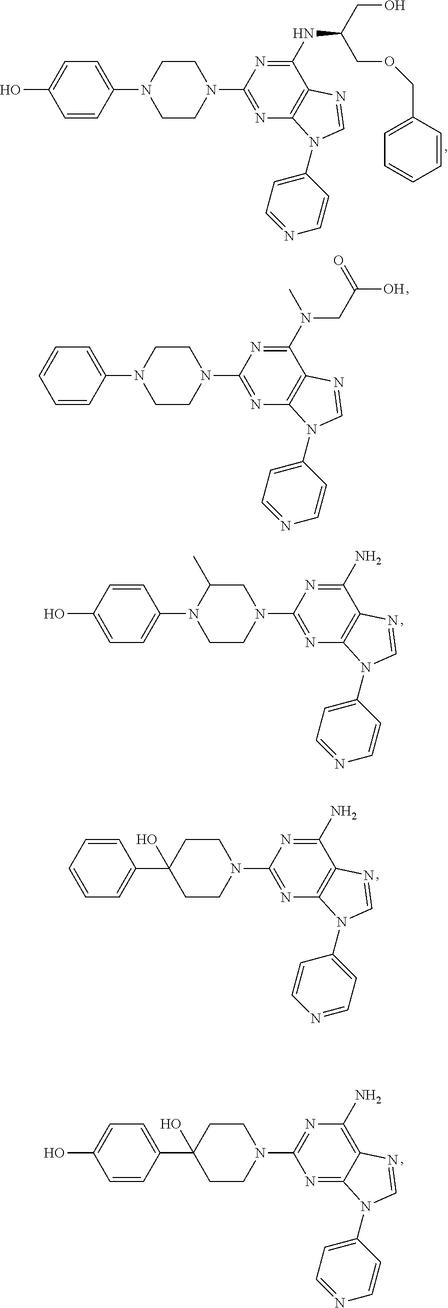

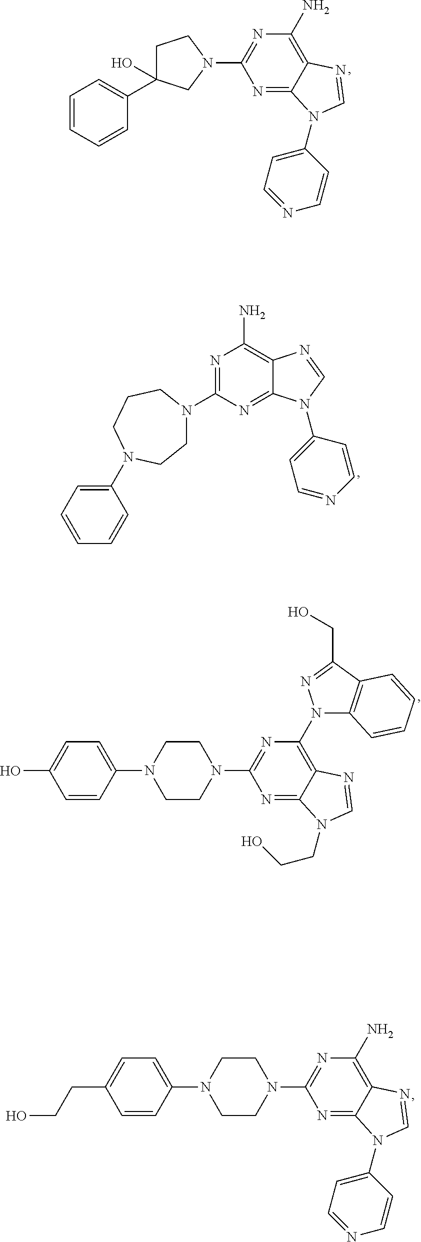 [SMILES: Nc1nc(N2CCC(O)(c3ccccc3)C2)nc2c1ncn2-c1ccncc1 ; Nc1nc(N2CCCN(c3ccccc3)CC2)nc2c1ncn2-c1ccncc1 ; OCCn1cnc2c(-n3nc(CO)c4ccccc43)nc(N3CCN(c4ccc(O)cc4)CC3)nc21 ; Nc1nc(N2CCN(c3ccc(CCO)cc3)CC2)nc2c1ncn2-c1ccncc1]

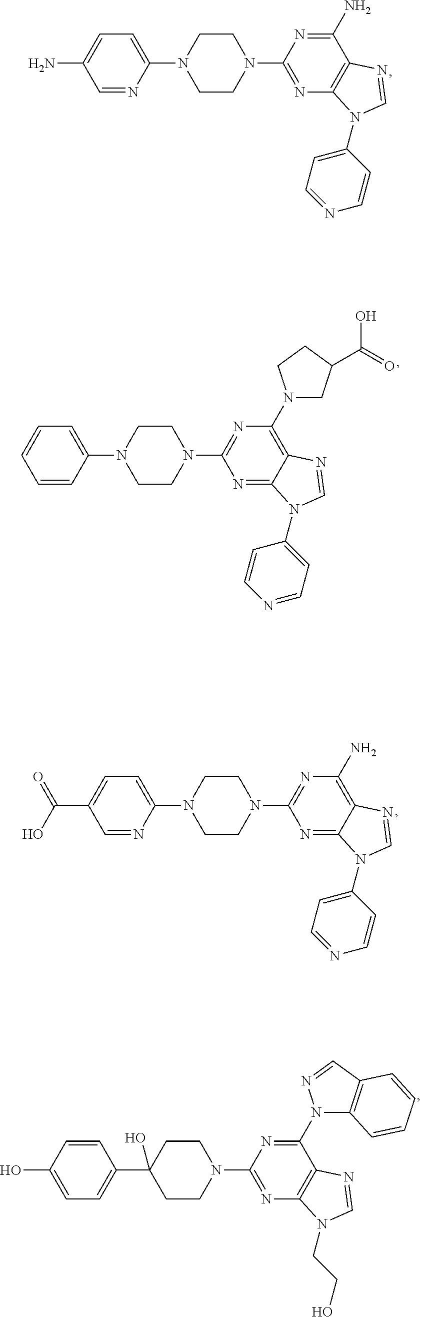 [SMILES: Nc1ccc(N2CCN(c3nc(N)c4ncn(-c5ccncc5)c4n3)CC2)nc1 ; O=C(O)C1CCN(c2nc(N3CCN(c4ccccc4)CC3)nc3c2ncn3-c2ccncc2)C1 ; Nc1nc(N2CCN(c3ccc(C(=O)O)cn3)CC2)nc2c1ncn2-c1ccncc1 ; OCCn1cnc2c(-n3ncc4ccccc43)nc(N3CCC(O)(c4ccc(O)cc4)CC3)nc21]

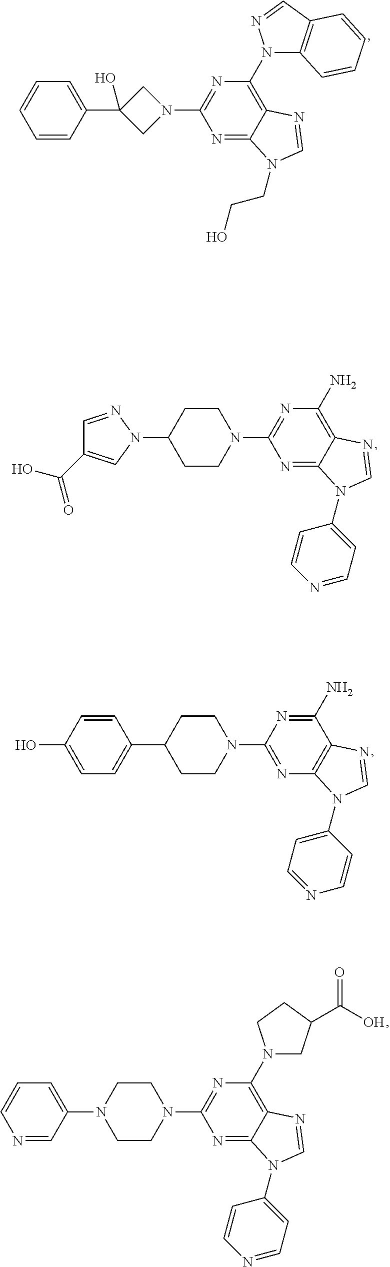 [SMILES: OCCn1cnc2c(-n3ncc4ccccc43)nc(N3CC(O)(c4ccccc4)C3)nc21 ; Nc1nc(N2CCC(n3cc(C(=O)O)cn3)CC2)nc2c1ncn2-c1ccncc1 ; Nc1nc(N2CCC(c3ccc(O)cc3)CC2)nc2c1ncn2-c1ccncc1 ; O=C(O)C1CCN(c2nc(N3CCN(c4cccnc4)CC3)nc3c2ncn3-c2ccncc2)C1]

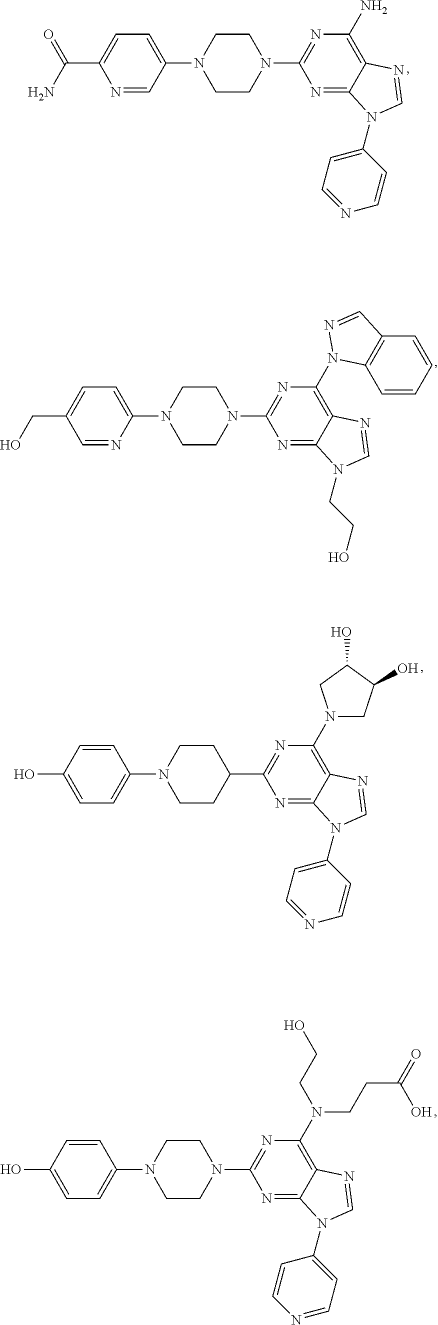 [SMILES: NC(=O)c1ccc(N2CCN(c3nc(N)c4ncn(-c5ccncc5)c4n3)CC2)cn1 ; OCCn1cnc2c(-n3ncc4ccccc43)nc(N3CCN(c4ccc(CO)cn4)CC3)nc21 ; Oc1ccc(N2CCC(c3nc(N4C[C@H](O)[C@@H](O)C4)c4ncn(-c5ccncc5)c4n3)CC2)cc1 ; O=C(O)CCN(CCO)c1nc(N2CCN(c3ccc(O)cc3)CC2)nc2c1ncn2-c1ccncc1]

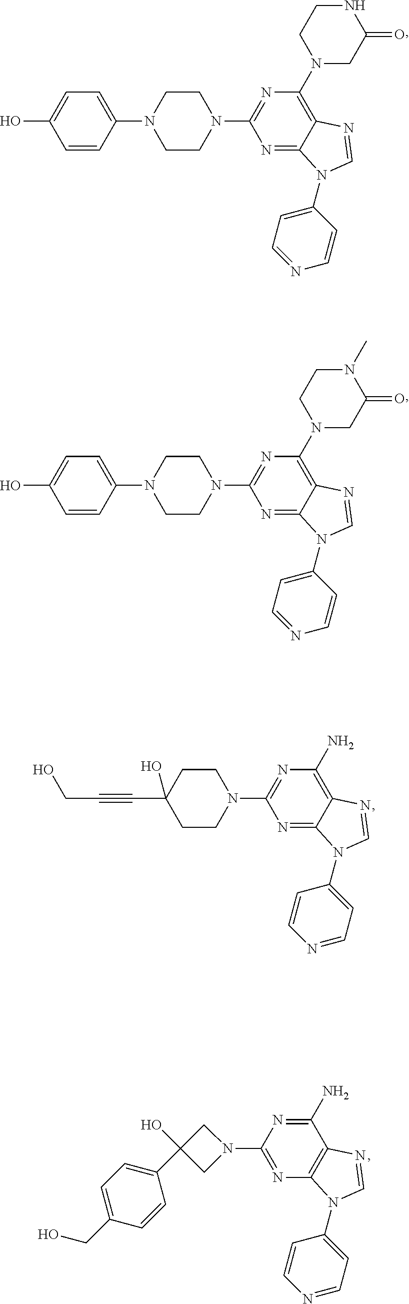 [SMILES: O=C1CN(c2nc(N3CCN(c4ccc(O)cc4)CC3)nc3c2ncn3-c2ccncc2)CCN1 ; CN1CCN(c2nc(N3CCN(c4ccc(O)cc4)CC3)nc3c2ncn3-c2ccncc2)CC1=O ; Nc1nc(N2CCC(O)(C#CCO)CC2)nc2c1ncn2-c1ccncc1 ; Nc1nc(N2CC(O)(c3ccc(CO)cc3)C2)nc2c1ncn2-c1ccncc1]

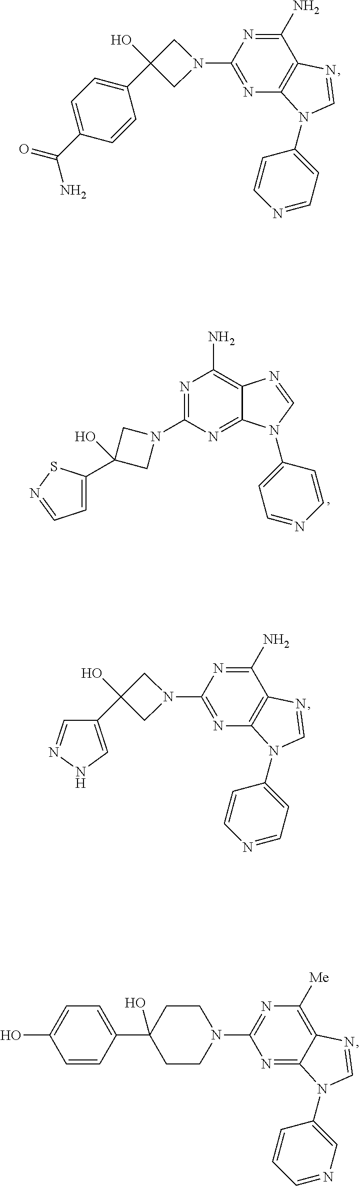 [SMILES: NC(=O)c1ccc(C2(O)CN(c3nc(N)c4ncn(-c5ccncc5)c4n3)C2)cc1 ; Nc1nc(N2CC(O)(c3ccns3)C2)nc2c1ncn2-c1ccncc1 ; Nc1nc(N2CC(O)(c3cn[nH]c3)C2)nc2c1ncn2-c1ccncc1 ; Cc1nc(N2CCC(O)(c3ccc(O)cc3)CC2)nc2c1ncn2-c1cccnc1]

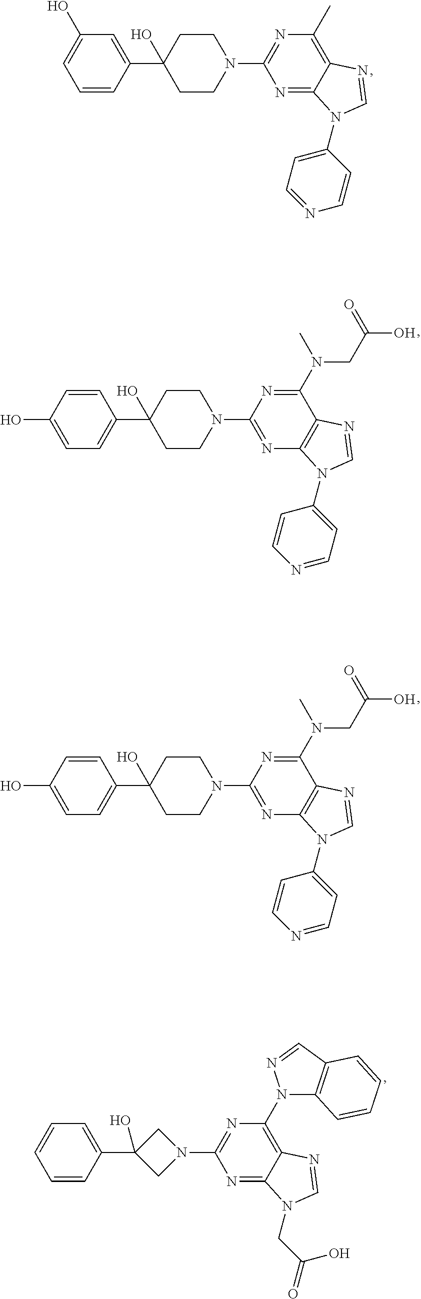 [SMILES: Cc1nc(N2CCC(O)(c3cccc(O)c3)CC2)nc2c1ncn2-c1ccncc1 ; CN(CC(=O)O)c1nc(N2CCC(O)(c3ccc(O)cc3)CC2)nc2c1ncn2-c1ccncc1 ; CN(CC(=O)O)c1nc(N2CCC(O)(c3ccc(O)cc3)CC2)nc2c1ncn2-c1ccncc1 ; O=C(O)Cn1cnc2c(-n3ncc4ccccc43)nc(N3CC(O)(c4ccccc4)C3)nc21]

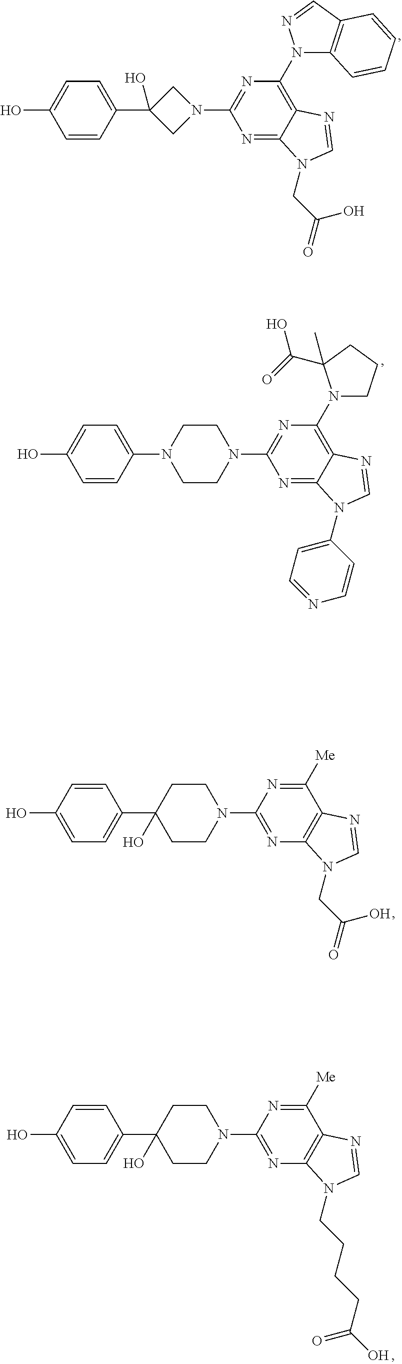 [SMILES: O=C(O)Cn1cnc2c(-n3ncc4ccccc43)nc(N3CC(O)(c4ccc(O)cc4)C3)nc21 ; CC1(C(=O)O)CCCN1c1nc(N2CCN(c3ccc(O)cc3)CC2)nc2c1ncn2-c1ccncc1 ; Cc1nc(N2CCC(O)(c3ccc(O)cc3)CC2)nc2c1ncn2CC(=O)O ; Cc1nc(N2CCC(O)(c3ccc(O)cc3)CC2)nc2c1ncn2CCCCC(=O)O]

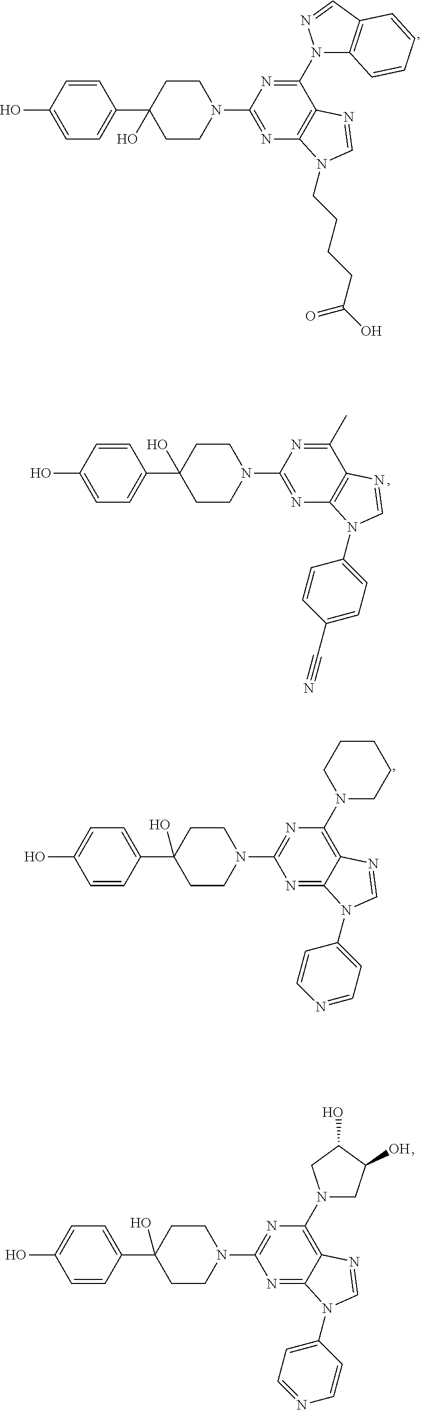 [SMILES: O=C(O)CCCCn1cnc2c(-n3ncc4ccccc43)nc(N3CCC(O)(c4ccc(O)cc4)CC3)nc21 ; Cc1nc(N2CCC(O)(c3ccc(O)cc3)CC2)nc2c1ncn2-c1ccc(C#N)cc1 ; Oc1ccc(C2(O)CCN(c3nc(N4CCCCC4)c4ncn(-c5ccncc5)c4n3)CC2)cc1 ; Oc1ccc(C2(O)CCN(c3nc(N4C[C@H](O)[C@@H](O)C4)c4ncn(-c5ccncc5)c4n3)CC2)cc1]

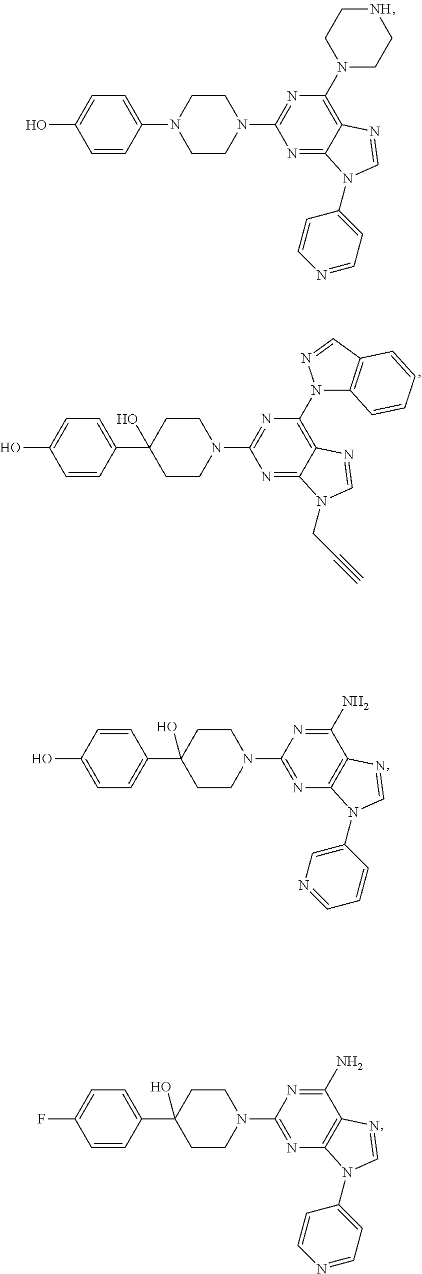 [SMILES: Oc1ccc(N2CCN(c3nc(N4CCNCC4)c4ncn(-c5ccncc5)c4n3)CC2)cc1 ; C#CCn1cnc2c(-n3ncc4ccccc43)nc(N3CCC(O)(c4ccc(O)cc4)CC3)nc21 ; Nc1nc(N2CCC(O)(c3ccc(O)cc3)CC2)nc2c1ncn2-c1cccnc1 ; Nc1nc(N2CCC(O)(c3ccc(F)cc3)CC2)nc2c1ncn2-c1ccncc1]

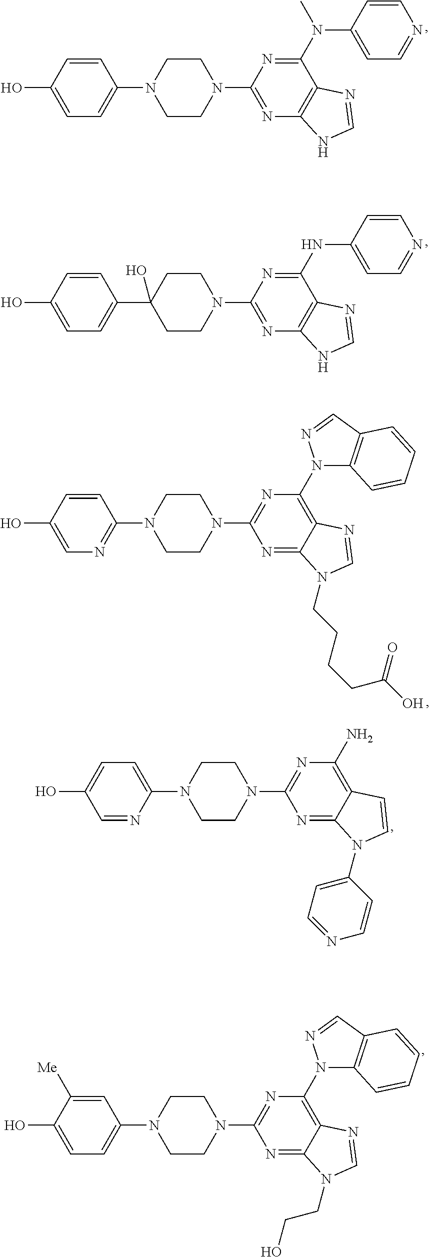

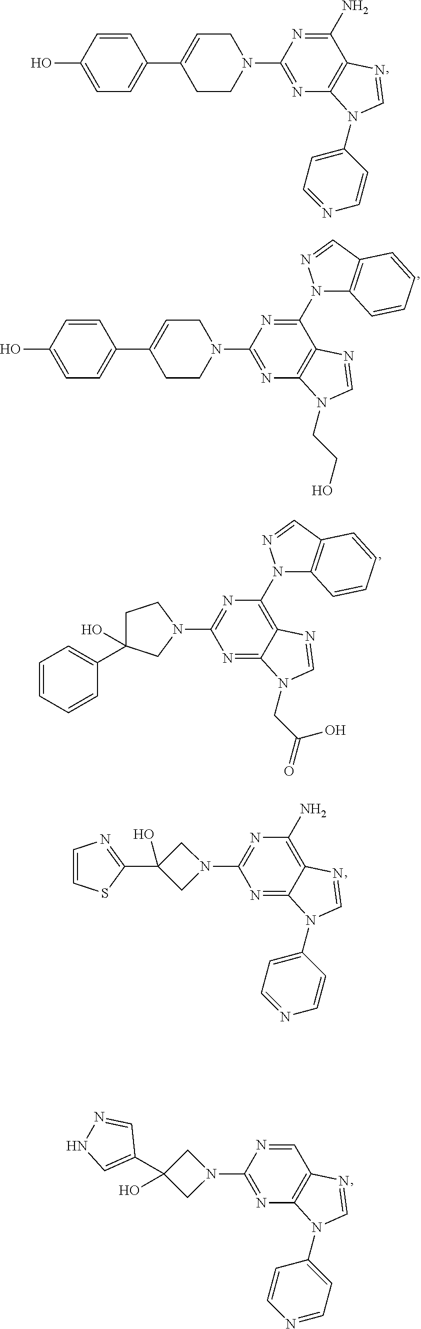

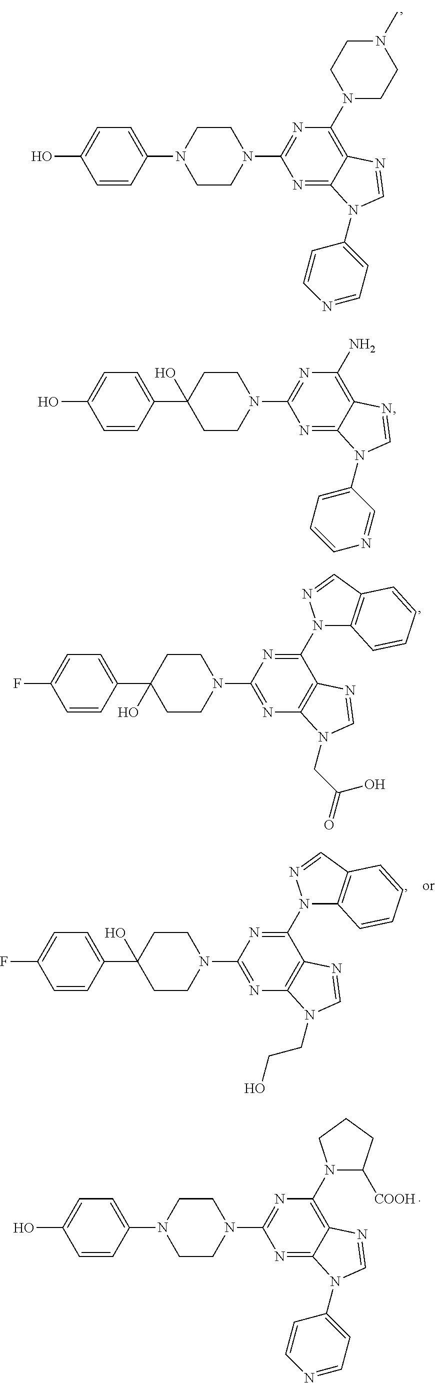

In another aspect, the present invention provides pharmaceutical compositions, each including any one of the compounds described or shown above, or a pharmaceutically acceptable salt, solvent, isomer, or prodrug, and a pharmaceutically acceptable carrier.

In some embodiments, each of the pharmaceutical compositions is provided in the form of a solution, a pill, a capsule or powder. The pharmaceutical compositions can be administered by means well known in the art, e.g., orally or parenterally (e.g., intravenously).

Yet in another aspect, the present invention provides methods for treating a disorder mediated by macrophage migration inhibitory factor in a subject, which includes administering to the subject in need thereof any of the compounds or pharmaceutical compositions described or shown above. Such a disorder can be an inflammatory disease or cancer. Examples of the inflammatory or autoimmune disease include asthma or rheumatoid arthritis, and examples of the cancer include gastric cancer, pancreatic cancer, melanoma, hepatocellular carcinoma, malignant glioma, and cervical adenocarcinoma.

In some embodiments, the subject is a mammal (e.g., a human being).

### BRIEF DESCRIPTIONS OF THE DRAWINGS

FIG. 1 shows the effect of Example 100 on MIF tautomerase activity.

FIG. 2 shows the effect of Example 55 on MIF tautomerase activity.

FIG. 3 shows the effect of Example 109 on MIF tautomerase activity.

FIG. 4 shows the effect of Example 51 on MIF tautomerase activity.

FIG. 5 shows the effect of Example 99 on MIF tautomerase activity.

FIG. 6 shows the effect of Example 80 on BV2 cell proliferation.

FIG. 7 shows the effect of Example 81 on BV2 cell proliferation.

FIG. 8 shows the effect of Example 95 on BV2 cell proliferation.

FIG. 9 shows the effect of Example 51 on MIF-CD74 binding.

FIG. 10 shows the effect of Example 62 on MIF-CD74 binding.

FIG. 11 shows the effect of Example 115 on MIF-CD74 binding.

FIG. 12 shows the effect of Example 148 on MIF-CD74 binding.

FIG. 13 shows the inhibition of breast cancer growth by compound of this invention.

### DETAILED DESCRIPTION OF THE INVENTION

As used herein, the term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl” Alkyl groups that are limited to hydrocarbon groups are termed “homoalkyl”. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀alkyl (e.g., —CH₃). In certain embodiments, the alkyl group is substituted C₁₋₁₀alkyl. Common alkyl abbreviations include Me (—CH₃), Et(—CH₂CH₃), nPr(—CH(CH₃)₂), nPr(—CH₂CH₂CH₃), n-Bu (—CH₂CH₂CH₂CH₃), or i-Bu (—CH₂CH(CH₃)₂).

As used herein, the term “alkylene” by itself or as part of another substituent means a divalent radical derived from an alkane, as exemplified, but not limited, by —CH₂CH₂CH₂CH₂—, and further includes those groups described below as “heteroalkylene” Typically, an alkyl (or alkylene) group will have from 1 to 24 carbon atoms, with those groups having 10 or fewer carbon atoms being preferred in the present invention A “lower alkyl” or “lower alkylene” is a shorter chain alkyl or alkylene group, generally having eight or fewer carbon atoms.

As used herein, the term “alkenyl” refers to a straight or branched hydrocarbon chain containing 2-12 carbon atoms (unless otherwise noted) and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent.

As used herein, the term “alkenylene” refers to a divalent alkenyl, e.g. —CH═CH—, —CH₂—CH═CH—, and —CH═CH—CH₂—.

As used herein, the term “alkynyl” refers to a straight or branched hydrocarbon chain containing 2-12 carbon atoms (unless otherwise noted) and characterized in having one or more triple bonds. Examples of alkynyl groups include, but are not limited to, ethynyl, propargyl, and 3-hexynyl. One of the triple bond carbons may optionally be the point of attachment of the alkynyl substituent.

As used herein, the term “alkynylene” refers to a divalent alkynyl, e.g. —CH≡CH—, —CH₂—CH≡CH—, and —CH≡CH—CH₂—.

As used herein, the terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

As used herein, the terms “cyano” and “nitrile” refer to the radical —CN.

As used herein, the terms “cycloalkyl”, “heterocycloalkyl” or “heterocyclyl”, by themselves or in combination with other terms, represent, unless otherwise stated, cyclic versions of “alkyl” and “heteroalkyl”, respectively. Additionally, for heterocycloalkyl or heterocyclyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, cyclooctanyl, and the like. Examples of heterocycloalkyl and heterocyclyl include, but are not limited to, 1-1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

As used herein, the term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, the terms “heterocyclyl” when used in combination with other terms (e.g., heterocyclylalkyl) includes heterocyclyl rings as defined above. Thus, the term “heterocyclylalkyl” is meant to include those radicals in which a heterocyclyl group is attached to an alkyl group including those alkyl groups in which a carbon atom (e.g., a methylene group) has been replaced by, for example, an oxygen atom.

As used herein, the terms “halo” or “halogen,” by themselves or as part of another substituent, mean, unless otherwise stated, a fluorine, chlorine, bromine, or iodine atom.

As used herein, the term “haloalkyl,” as used herein, refers to an alkyl group, as defined herein, which further comprises 1 or more (e.g., 1, 2, 3, or 4) halogen atoms (e.g., fluorine, chlorine, bromine, or iodine), wherein the alkyl group is substituted with one or more halogen atoms. In certain embodiments, a haloalkyl group refers to a saturated group having from 1 to 10 carbon atoms and 1, 2, 3, or 4 halogen atoms (“haloC₁₋₁₀ alkyl”). Additionally, the term “haloalkyl,” is meant to include monohaloalkyl and polyhaloalkyl. For example, the term “haloalkyl” is mean to include, but not be limited to, trifluoromethyl, 2,2,2-trifluoroethyl, 4-chlorobutyl, 3-bromopropyl, and the like.

As used herein, the terms “haloalkoxy” or “haloalkoxyl” as used herein, refer to an alkoxy group, as defined herein, which further comprises 1 or more (e.g., 1, 2, 3, or 4) halogen atoms (e.g., fluorine, chlorine, bromine, or iodine), wherein the alkoxy group is substituted with one or more halogen atoms.

As used herein, the term “hydroxy” or “hydroxyl” refers to the radical —OH.

As used herein, the term “aryl” means, unless otherwise stated, a polyunsaturated, aromatic, hydrocarbon substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. For brevity, the term “aryl” when used in combination with other terms (e.g., aryloxy, arylthioxy, arylalkyl, aralkyl, heteroaralkyl) includes both aryl and heteroaryl rings as defined above. Thus, the terms “arylalkyl”, “aralkyl” and “heteroaralkyl” are meant to include those radicals in which an aryl or heteroaryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

As used herein, the term “nitro” refers to the radical —NO₂.

As used herein, the term “protecting group” refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., Protective Groups in Organic Synthesis, John Wiley & Sons, New York, 1991.

Alkyl, alkenyl, alkynyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds described herein that results in the formation of a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —ORaa, —ON(Rbb)₂, —N(Rbb)₂, —N(Rbb)₃+X, —N(ORcc)Rbb, —SH, —SR, —SSRcc, —C(═O)Raa, —CO₂H, —CHO, —C(ORcc)₂, —CO₂Raa, —OC(═O)Raa, —OCO₂Raa, —C(═O)N(Rbb)₂, —OC(═O)N(Rbb)₂, —NRbbC(═O)Raa, —NRbbCO₂Raa, —NRbbC(═O)N(Rbb)₂, —C(═NRbb)Ra, —C(═NRbb)ORaa, —OC(═NRbb)Raa, —OC(═NRbb)ORaa, —C(═NRbb)N(Rbb)₂, —OC(═NRbb)N(Rbb)₂, —NRbbC(═NRbb)N(Rbb)₂, —C(═O)NRbbSO₂Raa, —NRbbSO₂Raa, —SO₂N(Rbb)₂, —SO₂Raa, —SO₂ORaa, —OSO₂Raa, —S(O)Raa, —S(═O)Raa, —OS(═O)Raa, —Si(Raa)₃, —OSi(Raa)₃-C(═S)N(Rbb)₂, —C(═O)SRaa, —C(═S)SRaa, —SC(═S)SRaa, —SC(═O)SRaa, —OC(═O)SRaa, —SC(═O)ORaa, —SC(═O)Raa, —P(═O)₂R, —OP(═O)₂Raa, —P(═O)(Raa)₂, —OP(═O)(Raa)₂, —OP(═O)(ORcc)₂, —P(═O)₂N(Rbb)₂, —OP(═O)₂N(Rbb)₂, —P(═O)(NRbb)₂, —OP(═O)(NRbb)₂, —NRbbP(═O)(ORcc)₂, —NRbbP(═O)(NRbb)₂, —P(Rcc)₂, —P(Rcc)₃, —OP(Rcc)₂, —OP(Rcc)₃, —B(Raa)₂, —B(ORcc)₂, —BRaa(ORcc), C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl, and 5-14 membered heteroaryl, wherein each alkyl, alkenyl, alkynyl, cycloalkyl, heterocyclyl, aryl, and heteroaryl is independently substituted with 0, 1, 2, 3, 4, or 5 Rdd groups; each instance of Ra is, independently, selected from C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl, and 5-14 membered heteroaryl, or two Raa groups are joined to form a 3-14 membered heterocyclyl or 5-14 membered heteroaryl ring, wherein each alkyl, alkenyl, alkynyl, cycloalkyl, heterocyclyl, aryl, and heteroaryl is independently substituted with 0, 1, 2, 3, 4, or 5 Rdd groups; each instance of Rbb is, independently, selected from hydrogen, —OH, —ORaa, —N(Rcc)₂, —CN, —C(═O)Ra, —C(═O)N(Rcc)₂, —CO₂R, —SO₂Raa, —C(═NRcc)ORaa, —C(═NRcc)N(Rcc)₂, —SO₂N(Rcc)₂, —SO₂Rcc, —SO₂ORcc, —SORaa, —C(═S)N(Rcc)₂, —C(═O)SRcc, —C(═S)SRcc, —P(═O)₂R, —P(═O)(Ra)₂, —P(═O)₂N(Rcc)₂, —P(═O)(NRcc)₂, C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl, and 5-14 membered heteroaryl, or two Rbb groups are joined to form a 3-14 membered heterocyclyl or 5-14 membered heteroaryl ring, wherein each alkyl, alkenyl, alkynyl, cycloalkyl, heterocyclyl, aryl, and heteroaryl is independently substituted with 0, 1, 2, 3, 4, or 5 Rdd groups; each instance of Rcc is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ cycloalkyl, 3-14 membered heterocyclyl, C₆₋₁₄ aryl, and 5-14 membered heteroaryl, or two Rcc groups are joined to form a 3-14 membered heterocyclyl or 5-14 membered heteroaryl ring, wherein each alkyl, alkenyl, alkynyl, cycloalkyl, heterocyclyl, aryl, and heteroaryl is independently substituted with 0, 1, 2, 3, 4, or 5 Rdd groups; each instance of Rdd is, independently, selected from halogen, —CN, —NO₂, —N3, —SO₂H, —SO₃H, —OH, —ORee, —ON(Rff)₂, —N(Rff)₂, —N(Rff)₃+X, —N(ORee)Rff, —SH, —SRee, —SSRee, —C(═O)Ree, —CO₂H, —CO₂Ree, —OC(═O)Ree, —OCO₂Ree, —C(═O)N(Rff)₂, —OC(═O)N(Rff)₂, —NRffC(═O)Ree, —NRffCO₂Ree, —NRffC(═O)N(Rff)₂, —C(═NRff)ORee, —OC(═NRff)Ree, OC(═NRff)ORee, —C(═NRff)N(Rff)₂, —OC(═NRff)N(Rff)₂, —NRffC(═NRff)N(Rff)₂, —NRffSO₂Ree, —SO₂N(Rff)₂, —SO₂Ree, —SO₂ORee, —OSO₂Ree, —S(═O)Ree, —Si(Ree)₃, —OSi(Ree)₃, —C(═S)N(Rff)₂, —C(═O)SRee, —C(═S)SRee, —SC(═S)SRee, —P(═O)₂Ree, —P(═O)(Ree)₂, —OP(═O)(Ree)₂, —OP(═O)(ORee)₂, C₁₋₆ alkyl, C₁₋₆ perhaloalkyl, C₂₋₆ alkenyl, C₂₋₆ alkynyl, C₃₋₁₀ cycloalkyl, 3-10 membered heterocyclyl, C₆₋₁₀ aryl, 5-10 membered heteroaryl, wherein each alkyl, alkenyl, alkynyl, cycloalkyl, heterocyclyl, aryl, and heteroaryl is independently substituted with 0, 1, 2, 3, 4, or 5 Rgg groups, or two geminal Rdd substituents can be joined to form ═O or ═S; each instance of Ree is, independently, selected from C₁₋₆ alkyl, C₁₋₆ perhaloalkyl, C₂₋₆ alkenyl, C₂₋₆ alkynyl, C₃₋₁₀ cycloalkyl, C₆₋₁₀ aryl, 3-10 membered heterocyclyl, and 3-10 membered heteroaryl, wherein each alkyl, alkenyl, alkynyl, cycloalkyl, heterocyclyl, aryl, and heteroaryl is independently substituted with 0, 1, 2, 3, 4, or 5 Rgg groups; each instance of Rff is, independently, selected from hydrogen, C₁₋₆ alkyl, C₁₋₆ perhaloalkyl, C₂₋₆ alkenyl, C₂₋₆ alkynyl, C₃₋₁₀ cycloalkyl, 3-10 membered heterocyclyl, C₆₋₁₀ aryl and 5-10 membered heteroaryl, or two Rff groups are joined to form a 3-14 membered heterocyclyl or 5-14 membered heteroaryl ring, wherein each alkyl, alkenyl, alkynyl, cycloalkyl, heterocyclyl, aryl, and heteroaryl is independently substituted with 0, 1, 2, 3, 4, or 5 Rgg groups; and each instance of Rgg is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X—, —NH(C₁₋₆ alkyl)₂+X—, —NH2(C₁₋₆ alkyl)+X—, —NH₃+X—, —N(OC₁₋₆ alkyl(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═OC₁₋₆ alkyl), —N(C₁₋₆ alkyl)C(═OC₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C¹⁻⁶ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ cycloalkyl, C₆₋₁₀ aryl, 3-10 membered heterocyclyl, 5-10 membered heteroaryl; or two geminal Rgg substituents can be joined to form ═O or ═S; wherein X is a counterion.

These and other exemplary substituents are described in more detail in the Detailed Description, examples, and claims. The invention is not intended to be limited in any manner by the above exemplary listing of substituents.

In some embodiments, a compound described herein is formed into a salt. A compound described herein can be administered as a free acid, a zwitterion or as a salt. A salt can also be formed between a cation and a negatively charged substituent on a compound described herein. Suitable cationic counterions include sodium ions, potassium ions, magnesium ions, calcium ion, and ammonium ions (e.g., a tetraalkyl ammonium cation such as tetramethylammonium ion). In compounds including a positively charged substituent or a basic substituent, a salt can be formed between an anion and a positively charged substituent (e.g., amino group) or basic substituent (e.g., pyridyl) on a compound described herein. Suitable anions include chloride, bromide, iodide, sulfate, nitrate, phosphate, citrate, methanesulfonate, trifluoroacetate, and acetate.

Pharmaceutically acceptable salts of the compounds described herein (e.g., a compound of Formula (I) also include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, trifluoroacetate, and undecanoate.

Salts derived from appropriate bases include alkali metal (e.g., sodium), alkaline earth metal (e.g., magnesium), ammonium and N-(alkyl)₄+salts. This invention also envisions the quaternization of any basic nitrogen-containing groups of the compounds disclosed herein. Water or oil-soluble or dispersible products may be obtained by such quaternization.

As used herein, the compounds of this invention, including the compounds of Formula (I), are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood), or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

Any formula or a compound described herein is also intended to represent unlabeled forms as well as isotopically labeled forms of the compounds, isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ⁵¹P, ³²P, ³⁵S, ³⁶Cl, ¹²⁵I respectively. The invention includes various isotopically labeled compounds as defined herein, for example, those into which radioactive isotopes, such as ³H, ¹³C, and ¹⁴C are present. Such isotopically labelled compounds are useful in metabolic studies (with ¹⁴C), reaction kinetic studies (with, for example 'H or ³H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies, isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of a formula described herein. The concentration of such a heavier isotope, specifically deuterium, may be defined by the isotopic enrichment factor. The term “isotopic enrichment factor” as used herein means the ratio between the isotopic abundance and the natural abundance of a specified isotope if a substituent m a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99/o deuterium incorporation), or at least 8633.3 (99.5% deuterium incorporation).

Isotopically-labelled compounds described herein can generally be prepared by conventional techniques known to those skilled in the art or by processes analogous to those described in the accompanying Examples and Preparations using appropriate isotopically-labeled reagents in place of the non-labeled reagent previously employed. Pharmaceutically acceptable solvates in accordance with the invention include those wherein the solvent of crystallization may be isotopically substituted, e.g, D₂O, D₂-acetone, D₂-DMSO.

Any asymmetric atom (e.g., carbon or the like) of the compound(s) of the present invention can be present in racemic or enantiomerically enriched, for example the (R)-(S)- or (RS)-configuration, in certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form Accordingly, as used herein a compound of the present invention can be in the form of one of the possible isomers, rotamers, atropisomers, tautoroers or mixtures thereof, for example, as substantially pure geometric (cis or trans) isomers, diastereomers, optical isomers (antipodes), racemates or mixtures thereof. Any resulting mixtures of isomers can be separated on the basis of the physicochemical differences of the constituents, into the pure or substantially pure geometric or optical isomers, diastereomers, racemates, for example, by chromatography or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, (+)-O,O′-Di-p-toluoyl-D-tartaric acid, mandelic acid, malic acid or camphor-10-sulfonic acid. Racemic products can also be resolved by chiral chromatography, e.g., high pressure liquid chromatography (HPLC) using a chiral adsorbent.

The compounds described herein (e.g., a compound of Formula (I)) may also be represented in multiple tautomeric forms. In such instances, the invention expressly includes all tautomeric forms of the compounds described herein. All crystal forms of the compounds described herein are expressly included in this invention.

The compounds and pharmaceutical compositions of this invention (e.g., those of Formula (I)) can be evaluated for its ability to modulate (e.g., inhibit) macrophage migration inhibitory factor (MIF).

Synthesis of Compounds of this Invention

A. General Synthetic Procedures

Compound numbers 1-147 as used in the general synthesis section below refer only to genus structures in this section and do not apply to compounds disclosed elsewhere in this application. Compounds disclosed herein can be made by methods depicted in the reaction schemes below.

The starting materials and reagents used in preparing these compounds are either available from commercial supplier such as Aldrich Chemical Co., Bachem, etc., or can be made by methods well known in the art. The schemes are merely illustrative of some methods by which the compounds disclosed herein can be synthesized and various modifications to these schemes can be made and will be suggested to POSITA having referred to this disclosure. The starting materials and the intermediates and the final products of the reaction may be isolated and purified if desired using conventional techniques, including but not limited to filtration, distillation, crystallization, chromatography, and the like and may be characterized using conventional means, including physical constants and spectral data.

Unless specified otherwise, the reactions described herein take place at atmospheric pressure over a temperature range from about −78° C. to about 150° C.

Compounds of Formula (I) having the structure:

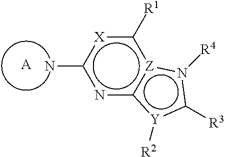

wherein X, Y, Z, Ring A, R¹, R², R³, and R⁴ are as defined in the Summary and the claims can be synthesized as illustrated and described in Scheme 1-6 below.

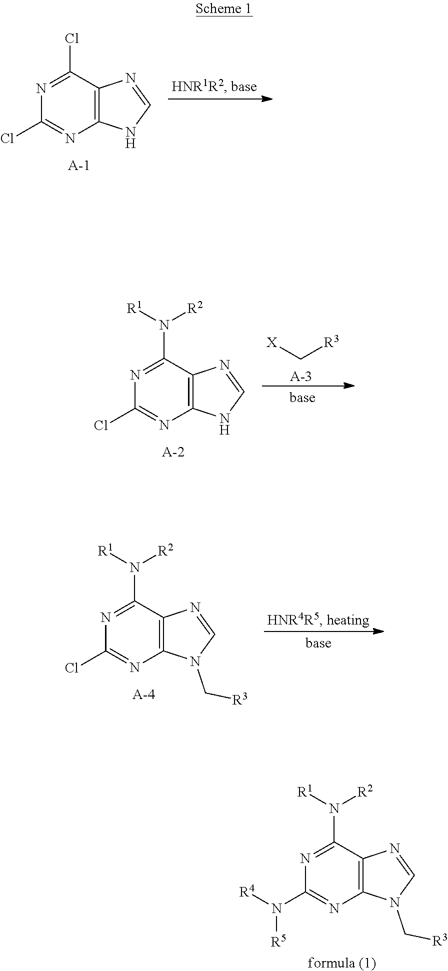

Amine A-2 is prepared from commercially available 2,6-dichloropurine (A-1) via regio-selective nucleophilic displacement of the 6-chloro group with a primary or secondary amine in the presence of a base such as K₂CO₃, Cs₂CO₃, Et₃N, and i-PrNEt₂, in a suitable solvent, such as DMF, DMSO, NMP, THF, EtOH, i-PrOH or t-BuOH. Treatment of intermediate A-2 with alkylating reagent A-3 where X is halogen, OMs, OTs or OTf and R³ is CH₂R⁶, CH₂CH₂R⁶, CH₂CO₂R⁶, CH₂CO₂R⁶, CH₂CH₂CO₂R⁶ where R⁶ is ORa, SRa, SO₂Ra, NRaRb, CO₂Ra, CONRaRb, alkyl group, aryl group, heterocycloalkyl group and heteroaryl group where R⁷ and R⁸ are H or alkyl groups, in the presence of a suitable base such as K₂CO₃, Cs₂CO₃, DBU, NaH, t-BuOK, and tetrabutylaminofluoride (TBAF), in a suitable solvent such as DMF and THE to provide the N-9 alkylated products A-4. Displacements of the 2-chloro groups in A-4 with a primary or secondary amine in a suitable solvent, such as DMSO, DMF, NMP, i-PrOH or t-BuOH provide the corresponding product of formula (1).

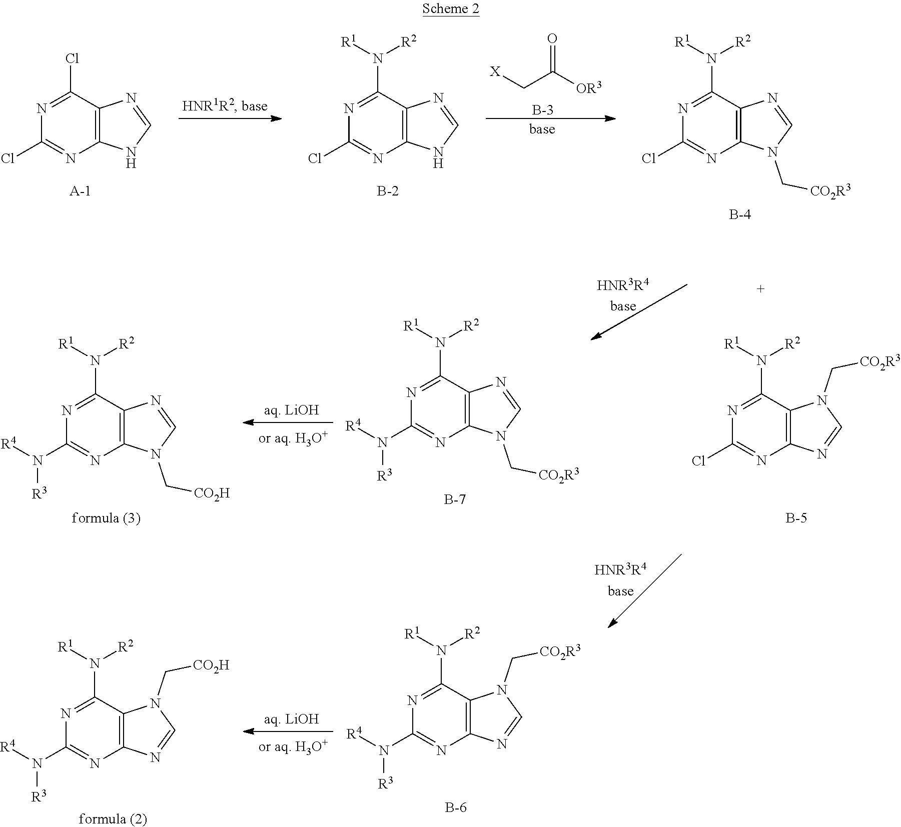

Amine B-2 is prepared from commercially available 2,6-dichloropurine (A-1) via regio-selective nucleophilic displacement of the 6-chloro group with a primary or secondary amine in the presence of a base such as K₂CO₃, Cs₂CO₃, Et₃N, and i-PrNEt₂, in a suitable solvent, such as DMF, DMSO, NMP, THF, EtOH, i-PrOH or t-BuOH. Treatment of intermediate B-2 with alkylating reagent B-3 where X is halogen, OMs, OTs or OTf and R³ is alkyl group in the presence of a suitable base such as K₂CO₃, Cs₂CO₃, DBU, NaH, t-BuOK, and tetrabutylaminofluoride (TBAF), in a suitable solvent such as DMF and THF to provide the N-7 and N-9 alkylated products (B-5 and B-4 respectively). Displacements of the 2-chloro groups in B4 and B-5 with a primary or secondary amine in a suitable solvent such as DMSO, DMF, NMP, i-PrOH or t-BuOH provide the corresponding products B-6 and B-7. Saponification of the corresponding esters (B-6 and B-7) with base such as aq. LiOH, aq. NaOH or aq. KOH in a suitable solvent such as THF, MeOH, or EtOH, or under the treatment of acid such as aq. TFA or aq. HCl in a solvent such as CH₂Cl₂, or MeOH, to provide the final products of formula (2) and formula (3), respectively.

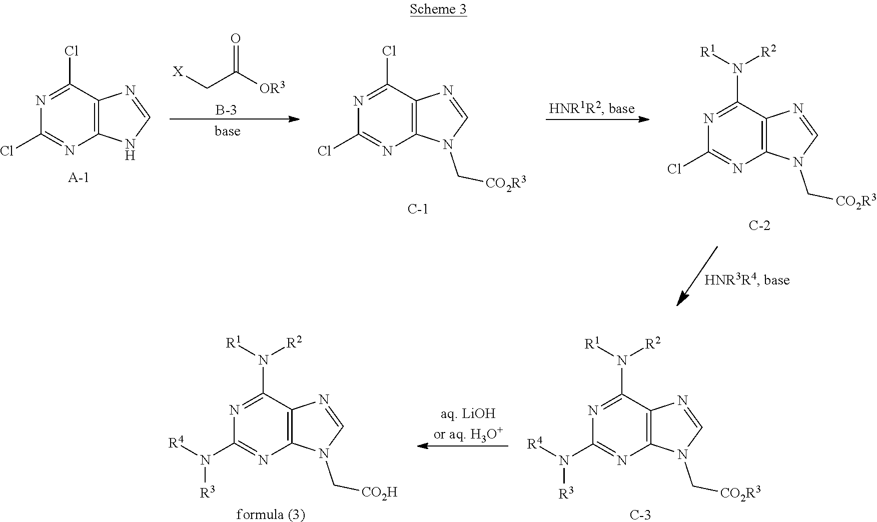

Compounds in formula (3) can also be prepared according to Scheme 3. Regio-selective alkylation of 2,6-dichloropurine (A-1) with alkylating reagent B-3 in the presence of a suitable base as described in Scheme 2 to provide the N-9 intermediate C-1. Sequential regio-selective displacements of the two chloro groups with amines such as HNR¹R² and HNR³R⁴ to intermediate C-2 first and then C-3. Removal of the ester group in C-3 can be achieved by either saponification with aq. LiOH, NaOH or KOH in THF or MeOH, or treatment with an acid such as TFA or HCl in water and MeOH, to provide the final product of formula (3).

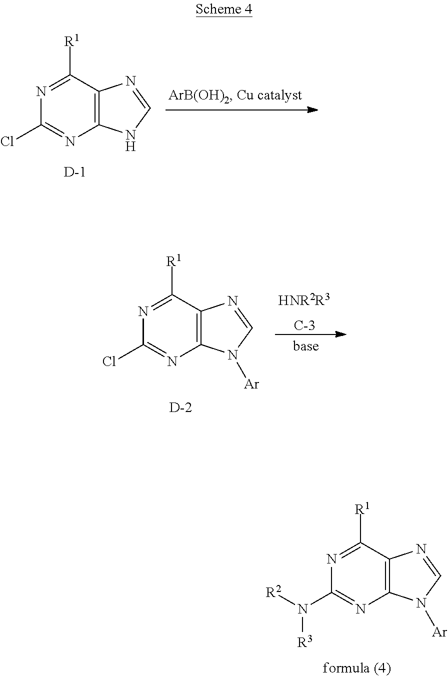

Compounds in formula (4) can also be prepared according to Scheme 4. Starting material D-1 where R¹ is alkyl group such as Me, Et, i-Pr, alkoxy group, aryl groups, heteroaryl group such as benzimidazoyl, indazoyl and benzotriazoyl, or amino group such as NH₂, NHRa and NRaRb where Ra and Rb are arayl, alkyl or cyclo-alkyl groups, can undergo an arylation with an arylboronic acid in the presence of a catalyst such as Cu₂(OAc)₄ to provide the product (D-2). A nucleophilic displacement of the 2-chloro group in D-2 with nucleophile HNR2R3 (C-3) where Ra and Rb are arayl, alkyl or cyclo-alkyl groups, in solvent such as THF, dioxane, DMSO, DMF, NMP, MeOH, EtOH or i-PrOH to provide the product of formula (4).

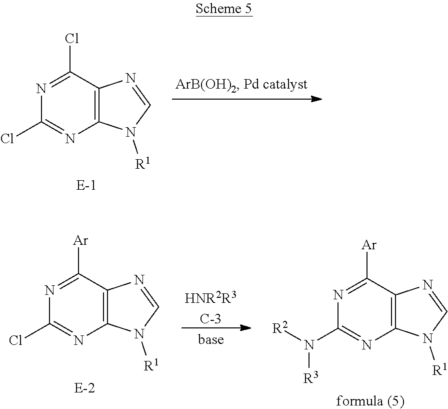

Compounds of formula (5) can also be prepared according to Scheme 5. Staring material E-1 where R¹ is alkyl group such as Me, Et, i-Pr, benzyl, CH₂-tetrazoyl, CH₂CH₂— tetrazoyl, CH₂CH₂OH, CH₂CH₂CH₂OH, CH₂CH₂NH₂, CH₂CH₂CH₂NH₂, CH₂CO₂Ra, CH₂CH₂CO₂Ra, CH₂CH₂CH₂CO₂Ra, or CH₂CONRaRb, CH₂CH₂CONRaRb, CH₂CH₂CH₂CONRaRb, where Ra and Rb are H, arayl, alkyl or cyclo-alkyl groups, can undergo a Suzuki coupling reaction with an arylboronic acid in the presence of a catalyst such as Pd in a suitable solvent, such as DMF, dioxane, THF, toluene and water to provide the product (E-2). A nucleophilic displacement of the 2-chloro group in C-2 with nucleophile HNR²R³(C-3) where Ra and Rb are arayl, alkyl or cyclo-alkyl groups, in solvent such as THF, dioxane, DMSO, DMF, NMP, MeOH, EtOH or i-PrOH to provide the product of formula (5).

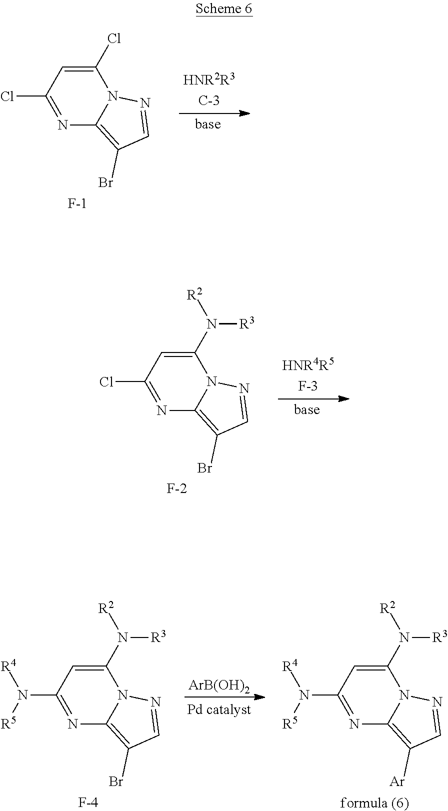

Compounds of formula (6) can also be prepared according to Scheme 6. Starting material F-1 is proceeded via two sequential nucleophilic displacements of the 2-chloro group with HNR²R³(C-3) and the 4-chloro group with HNR⁴R⁵ (F-3) where Ra, Rb, Rc and Rd are arayl, alkyl or cyclo-alkyl groups, in solvent such as THF, dioxane, DMSO, DMF, NMP, MeOH, EtOH or i-PrOH to provide intermediates F-2 and F-4 respectively. Installation of an aryl group via the Suzuki coupling reaction between the bromo group and an arylboronic acid can lead to the product of formula (6).

### SYNTHETIC EXAMPLES

### Example 1. 4-(4-(7-(2-hydroxyethyl)-4-(indolin-1-yl)-7H-pyrrolo[2,3-d]pyrimidin-2-yl)piperazin-1-yl)phenol

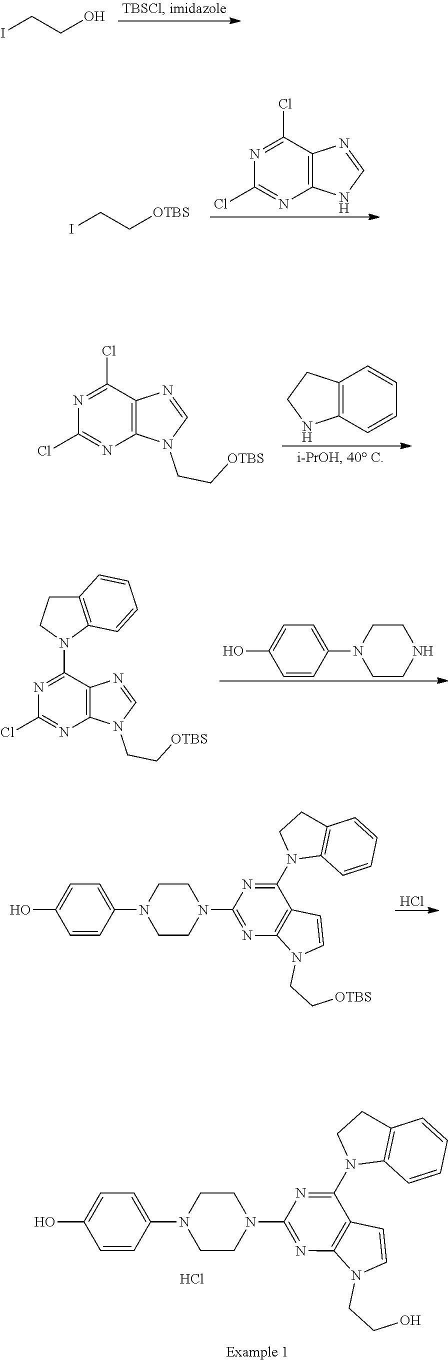

Step 1: To a mixture of 2-iodoethanol (10 g, 58.15 mmol, 1.0 eq) and imidazole (4.75 g, 69.78 mmol, 1.2 eq) in DCM (60 mL) at 0° C. was added TBSCl (9.2 g, 61.06 mmol, 1.05 eq) in portions. After stirring for 12 hours, the solution was washed with water (2×60 mL). The combined organic layer was washed with brine, dried over Na₂SO₄ and concentrated under vacuum to give tert-butyl(2-iodoethoxy)dimethylsilane as the desired product (16 g, 96% yield).

Step 2: To a mixture of tert-butyl(2-iodoethoxy)dimethylsilane (10 g, 34.92 mmol, 1.0 eq) and 2,6-dichloropurine (6.6 g, 34.92 mmol, 1.0 eq) in DMSO (60 mL) was added K₂CO₃ (12.1 g, 87.30 mmol, 2.5 eq). The mixture was stirred for 12 hours at 50° C. before it was allowed to cool and diluted with water (200 mL). The reaction mixture was extracted with EtOAc (2×100 mL). The combined organic layer was washed with water, brine, dried over Na₂SO₄, and concentrated. The residue was purified by flash silica gel column chromatography (petroleum ether:EtOAc=5:1) to give 9-(2-((tert-butyldimethylsilyl)oxy)ethyl)-2,6-dichloro-9H-purine as a white solid (5.5 g, 45% yield).

Step 3: To a mixture of 9-(2-((tert-butyldimethylsilyl)oxy)ethyl)-2,6-dichloro-9H-purine (1 g, 2.88 mmol, 1.0 eq) and indoline (0.412 g, 3.46 mmol, 1.2 eq) in EtOH (30 mL) was added Et3N (0.437 g, 4.32 mmol, 1.5 eq). The mixture was stirred for 6 hours at room temperature before the organic solvent was removed under reduced pressure. The residue was diluted with water (150 mL) and extracted with EtOAc (2×150 mL). The combined organic layer was washed with brine, dried over Na₂SO₄ and concentrated to give 9-(2-((tert-butyldimethylsilyl)oxy)ethyl)-2-chloro-6-(indolin-1-yl)-9H-purine as a white solid (1.15 g, 93% yield).

Step 4: To a solution of 9-(2-((tert-butyldimethylsilyl)oxy)ethyl)-2-chloro-6-(indolin-1-yl)-9H-purine (1.0 g, 2.33 mmol, 1.0 eq) in DMSO (15 mL) was added 4-(piperazin-1-yl)phenol (2.07 g, 11.63 mmol, 5.0 eq). The mixture was stirred for 6 hours at 100° C. under nitrogen atmosphere before it was allowed to cool and the solvent was removed under reduced pressure. The residue was diluted water (60 mL) and extracted with EtOAc (3×50 mL). The combined organic layer was washed with brine, dried over Na₂SO₄ and concentrated under vacuum. The residue was purified by flash silica gel column chromatography (petroleum ether:EtOAc=1:1) to give 4-(4-(7-(2-((tert-butyldimethylsilyl)oxy)ethyl)-4-(indolin-1-yl)-7H-pyrrolo[2,3-d]pyrimidin-2-yl)piperazin-1-yl)phenol as a white solid (0.80 g, 60% yield).

Step 5. To a solution of the product from the previous step (200 mg, 0.35 mmol, 1.0 eq) in MeOH (16 mL) at room temperature was added 1N aq. HCl solution (28 mL, 7 mmol, 20 eq). The mixture was stirred for 0.5 hour at room temperature as all of the starting material was consumed. The precipitate was collected by filtration and washed with EtOH to give 4-(4-(7-(2-hydroxyethyl)-4-(indolin-1-yl)-7H-pyrrolo[2,3-d]pyrimidin-2-yl)piperazin-1-yl)phenol hydrochloride as a white solid (112 mg, 70% yield).

¹H NMR (400 MHz, DMSO-d6): δ 13.30 (br, 1H), 10.06 (br s, 1H), 8.39 (s, 1H), 8.37 (s, 1H), 7.82-7.79 (m, 2H), 7.37-7.29 (m, 2H), 7.09-7.04 (m, 1H), 7.05-6.98 (m, 2H), 4.81-4.77 (m, 4H), 4.28-4.26 (m, 2H), 3.87-3.74 (m, 8H), 3.33 (t, J=8.4 Hz, 2H); LC/MS (ESI-MS): [M+H]+=458.1.

### Example 2. 1-(9-(2-hydroxyethyl)-2-(4-(4-hydroxyphenyl)piperazin-1-yl)-9H-purin-6-yl)indolin-2-one

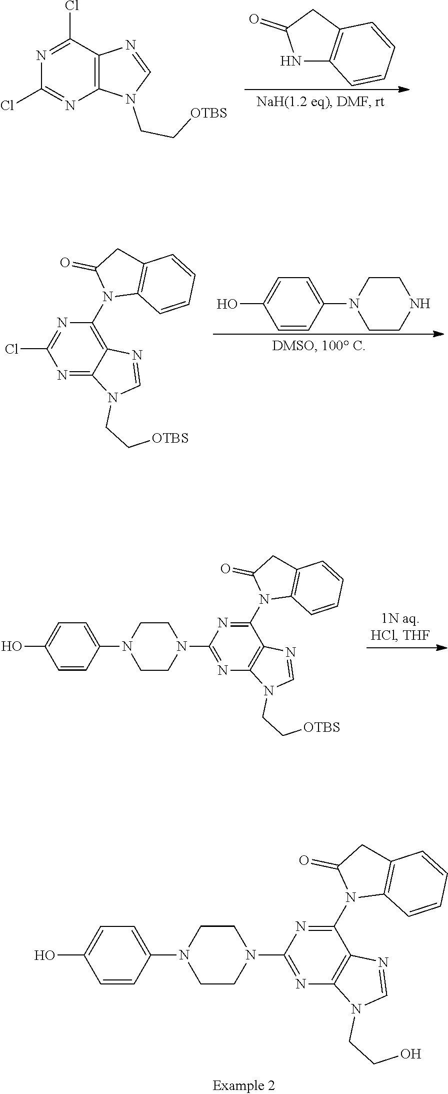 [SMILES: CC(C)(C)[Si](C)(C)OCCn1cnc2c(Cl)nc(Cl)nc21 ; O=C1Cc2ccccc2N1 ; CC(C)(C)[Si](C)(C)OCCn1cnc2c(N3C(=O)Cc4ccccc43)nc(Cl)nc21 ; Oc1ccc(N2CCNCC2)cc1 ; CC(C)(C)[Si](C)(C)OCCn1cnc2c(N3C(=O)Cc4ccccc43)nc(N3CCN(c4ccc(O)cc4)CC3)nc21 ; O=C1Cc2ccccc2N1c1nc(N2CCN(c3ccc(O)cc3)CC2)nc2c1ncn2CCO]

Step 1. To a mixture of indolin-2-one (372 mg, 2.8 mmol, 1 eq) in DMF (50 mL) at room temperature was added NaH (168 mg, 4.2 mmol, 60% in oil, 1.5 eq) in portions. The reaction mixture was stirred for 0.5 hours at room temperature and followed by addition of 9-(2-((tert-butyldimethylsilyl)oxy)ethyl)-2,6-dichloro-9H-purine (969 mg, 2.8 mmol, 1 eq). The mixture was stirred for 3 hours at room temperature before the solvent was removed under reduced pressure. The residue was diluted with water (50 mL) and extracted with EtOAc (2×50 mL). The combined organic layer was washed with brine, dried over Na₂SO₄ and concentrated to give the desired product 1-(9-(2-((tert-butyldimethylsilyl)oxy)ethyl)-2-chloro-9H-purin-6-yl)indolin-2-one as a white solid (1.2 g, 96% yield).

Step 2. To a solution of the product from the previous step (0.7 g, 1.57 mmol, 1 eq) in DMSO (20 mL) was added 4-(piperazin-1-yl)phenol (1.4 g, 7.9 mmol, 5 eq). The mixture was stirred for 1 h at 100° C. under nitrogen atmosphere before the solvent was removed under reduced pressure. The residue was diluted with water (50 mL) and the solid was collected by filtration. The crude solid was further washed with methanol to give the desired product 1-(9-(2-((tert-butyldimethylsilyl)oxy)ethyl)-2-(4-(4-hydroxyphenyl)piperazin-1-yl)-9H-purin-6-yl)indolin-2-one as a solid (300 mg, 41% yield).

Step 3: To a solution of the product from the previous step (300 mg, 0.63 mmol, 1 eq) in THF (10 mL) at room temperature was added 1N aq. HCl solution (12.6 mL, 12.6 mmol, 20 eq). The mixture was stirred for 0.5 hour at room temperature. The precipitate was collected by filtration and dried to provide 1-(9-(2-hydroxyethyl)-2-(4-(4-hydroxyphenyl)piperazin-1-yl)-9H-purin-6-yl)indolin-2-one hydrochloride as a white solid (150 mg, 50% yield).

¹H NMR (400 MHz, DMSO-d6): δ 11.01 (br, 1H), 8.92 (br, 1H), 8.23 (s, 1H), 7.67-7.65 (m, 2H), 7.00-6.89 (m, 6H), 4.21-4.19 (m, 4H), 3.81-3.79 (m, 4H), 3.77-3.70 (m, 6H); LC/MS (ESI-MS): [M+H]+=472.0.

### Example 3. 4-(4-(9-(2-hydroxyethyl)-6-(1H-indazol-1-yl)-9H-purin-2-yl)piperazin-1-yl)phenol

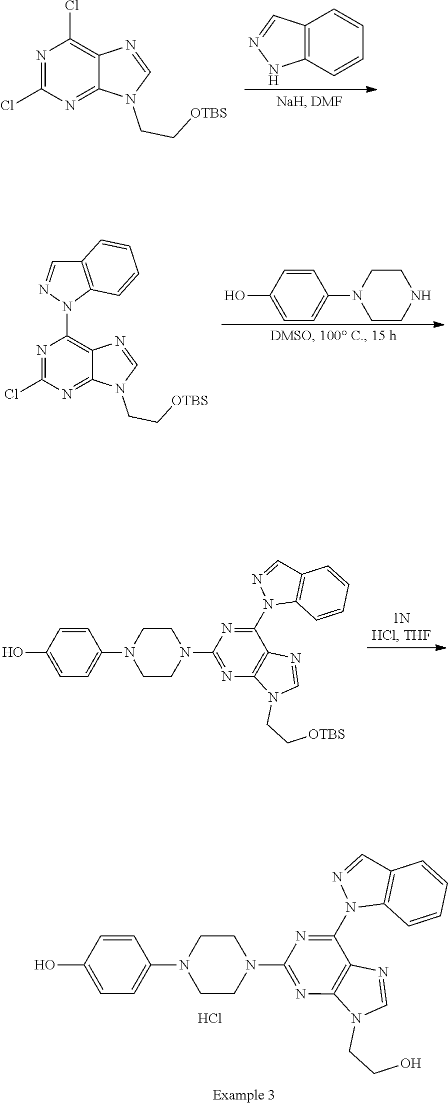

The proceeding example was prepared according to Example 2 substituting indolin-2-one with indazole. The desired product 4-(4-(9-(2-hydroxyethyl)-6-(1H-indazol-1-yl)-9H-purin-2-yl)piperazin-1-yl)phenol hydrochloride was isolated as a white solid.

¹H NMR (400 MHz, DMSO-d6): δ 9.28 (s, 1H), 8.79 (d, J=7.6 Hz, 1H), 8.02 (d, J=7.6 Hz, 2H), 7.73 (t, J=8 Hz, 1H), 7.51-7.48 (m, 3H), 6.87-6.84 (m, 3H), 4.39-4.36 (m, 4H), 3.89-3.81 (m, 4H), 3.70-3.45 (m, 4H); LC/MS (ESI-MS): [M+H]+=457.1.

### Example 4. (S)-4-(4-(9-(2-hydroxyethyl)-6-((1-phenylethyl)amino)-9H-purin-2-yl)piperazin-1-yl)phenol

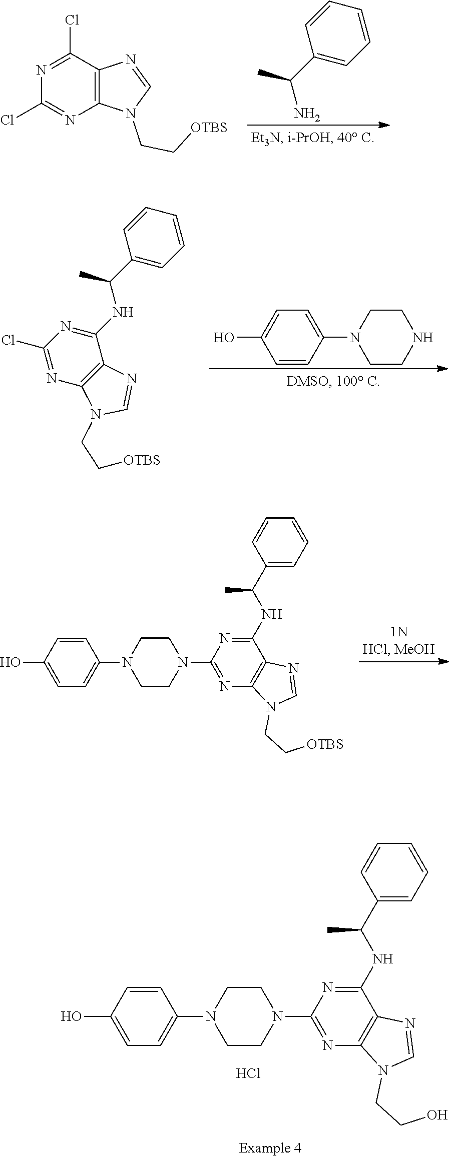

The proceeding example was prepared according to Example 1 substituting indoline with (S)-1-phenylethanamine. The desired product (S)-4-(4-(9-(2-hydroxyethyl)-6-((1-phenylethyl)amino)-9H-purin-2-yl)piperazin-1-yl)phenol hydrochloride was isolated as a white solid.

¹H NMR (400 MHz, DMSO-d6): δ 9.41 (br, 1H), 8.29-8.17 (m, 1H), 8.16-8.10 (m, 1H), 7.44 (d, J=7.2 Hz, 2H), 7.30 (t, J=7.6 Hz, 2H), 7.28-7.10 (m, 2H), 6.77 (d, J=8.4 Hz, 2H), 5.35-5.27 (m, 1H), 4.12 (s, 2H), 4.01-3.69 (m, 5H), 3.38-3.03 (m, 4H), 1.54 (d, J=6.8 Hz, 2H); LC/MS (ESI-MS): [M+H]+=460.1.

### Example 5. (R)-4-(4-(6-((1-hydroxy-3-phenylpropan-2-yl)amino)-9-(2-hydroxyethyl)-9H-purin-2-yl)piperazin-1-yl)phenol

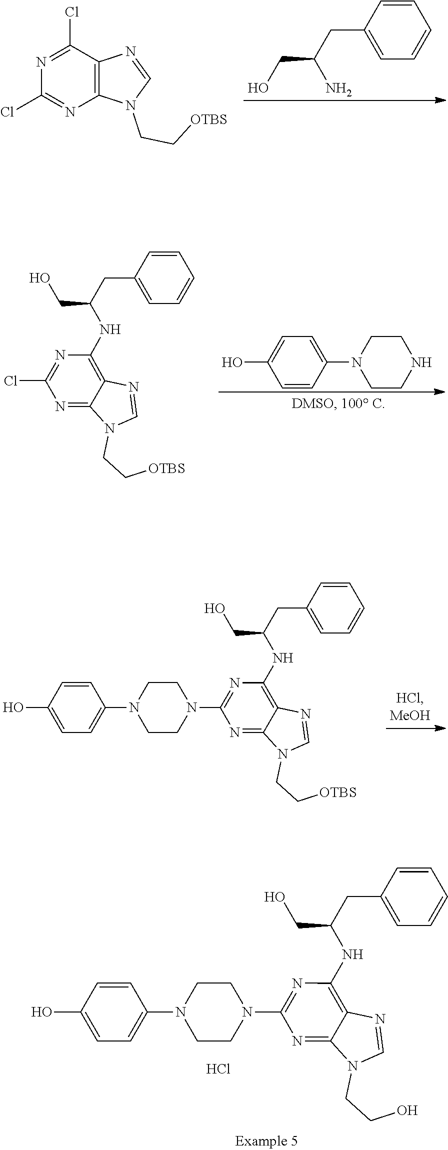

The proceeding example was prepared according to Example 1 substituting indoline with (R)-2-amino-3-phenylpropan-1-ol. The desired product (R)-4-(4-(6-((1-hydroxy-3-phenylpropan-2-yl)amino)-9-(2-hydroxyethyl)-9H-purin-2-yl)piperazin-1-yl)phenol hydrochloride was isolated as a white solid.

¹H NMR (400 MHz, DMSO-d6): δ 9.10 (s, 1H), 8.37 (br, 1H), 7.67 (br, 2H), 7.33-7.31 (m, 2H), 7.25-7.23 (m, 2H), 7.19-7.10 (m, 1H), 6.91-6.85 (m, 2H), 4.43-4.32 (m, 2H), 4.22-4.19 (m, 4H), 3.88-3.79 (m, 4H), 3.53-3.41 (m, 5H), 3.09-2.99 (m, 1H), 3.86-3.77 (m, 1H); LC/MS (ESI-MS): [M+H]+=490.2.

### Example 6. (R)-4-(4-(6-((2,3-dihydro-1H-inden-1-yl)amino)-9-(2-hydroxyethyl)-9H-purin-2-yl)piperazin-1-yl)phenol

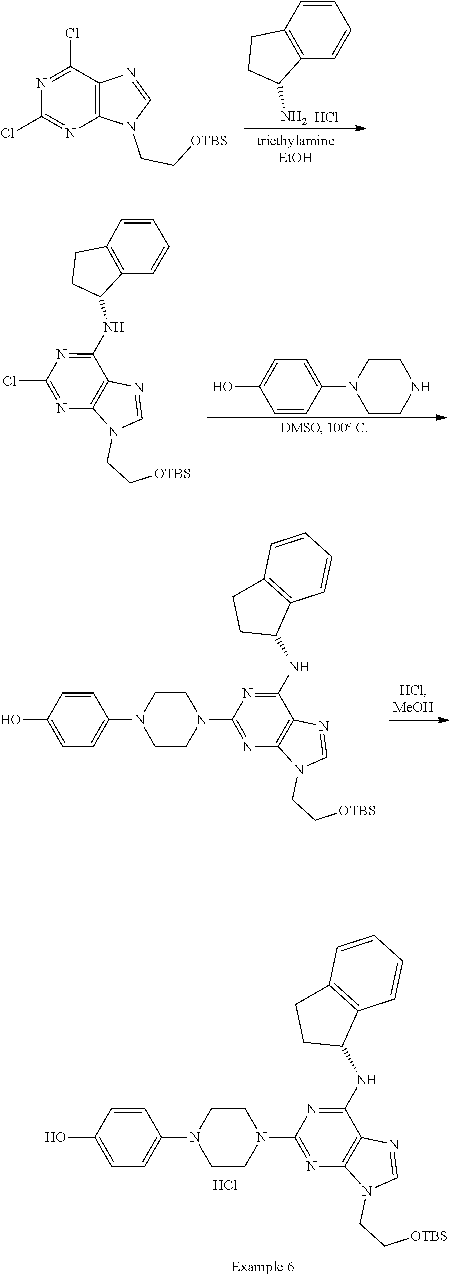

The proceeding example was prepared according to Example 1 substituting indoline with (R)-2,3-dihydro-1H-inden-1-amine hydrochloride. The desired product (R)-4-(4-(6-((2,3-dihydro-1H-inden-1-yl)amino)-9-(2-hydroxyethyl)-9H-purin-2-yl)piperazin-1-yl)phenol hydrochloride was isolated as a white solid.

¹H NMR (400 MHz, DMSO-d6): δ 10.11 (br, 1H), 9.01 (s, 1H), 8.81 (s, 1H), 7.68-7.51 (m, 3H), 7.33-7.16 (m, 4H), 6.87 (d, J=8.4 Hz, 2H), 5.79-5.77 (m, 1H), 4.88-4.41 (m, 2H), 4.27-4.21 (m, 3H), 3.80-3.76 (m, 3H), 3.60-3.31 (m, 5H), 3.06-2.99 (m, 1H), 2.91-2.87 (m, 1H), 2.57-2.54 (m, 1H), 2.08-2.03 (m, 1H); LC/MS (ESI-MS): [M+H]+=472.0.

### Example 7. 4-(4-(9-(3-hydroxypropyl)-6-(indolin-1-yl)-9H-purin-2-yl)piperazin-1-yl)phenol

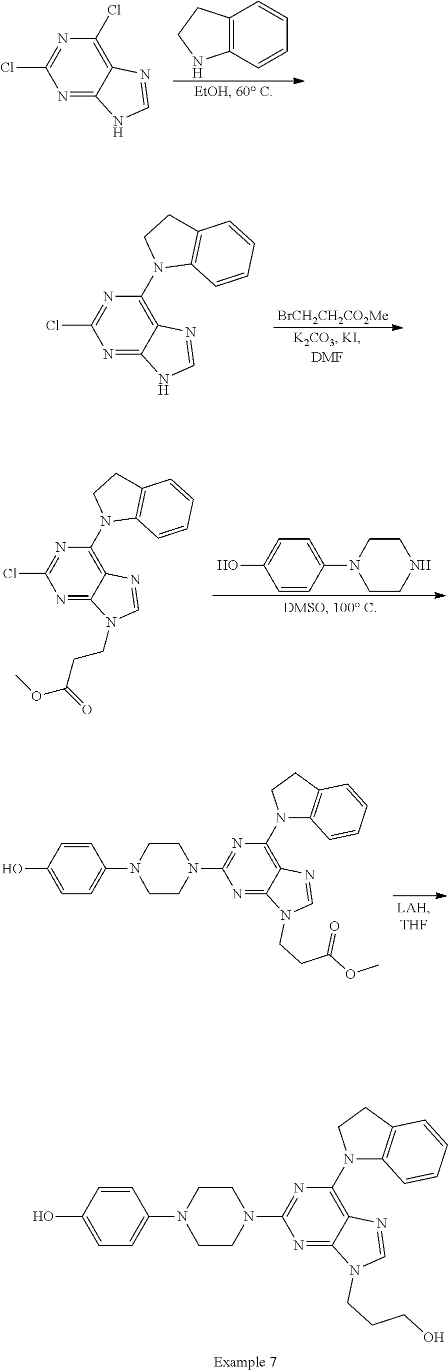

Step 1: A mixture of 2,6-dichloro-9H-purine (5.0 g, 26.46 mmol, 1.0 eq) and indoline (9.46 g, 79.37 mmol 3.0 eq) in EtOH (25 mL) was heated at 60° C. for 24 h before it was allowed to cool. The resulting precipitate was collected by filtration and dried to give 2-chloro-6-(indolin-1-yl)-9H-purine (5.56 g) as a white solid.

Step 2: To a solution of 2-chloro-6-(indolin-1-yl)-9H-purine (500 mg, 1.8 mmol, 1.0 eq) and BrCH₂CH₂CO₂Me (924 mg, 5.4 mmol 3.0 eq) in DMF (4 mL) at room temperature was added K₂CO₃ (918 mg, 6.5 mmol, 3.6 eq), KI (110 mg, 65 mmol, 0.4 eq). The resulting mixture was stirred at room temperature overnight before the reaction was diluted with water (20 mL) and extracted with EtOAc (2×30 mL). The combined organic layer was washed with water (3×60 mL) and brine (2×60 mL), dried over Na₂SO₄ and concentrated. The resulting residue was purified by flash silica gel column chromatography (petroleum ether:EtOAc=1:1) to give methyl 3-(2-chloro-6-(indolin-1-yl)-9H-purin-9-yl)propanoate as the desired product (100 mg, 16% yield).

Step 3: A mixture of methyl 3-(2-chloro-6-(indolin-1-yl)-9H-purin-9-yl)propanoate (100 mg, 0.279 mmol, 1 eq) and 4-(piperazin-1-yl)phenol (249 mg, 1.39 mmol, 5 eq) in DMSO (5 mL) was stirred at 100° C. overnight. The reaction mixture was allowed to cool and quenched with H₂O (4 mL). The resulting solid precipitate was collected by filtration. The crude product which was further slurried in DCM (3 mL), collected by filtration and dried to give methyl 3-(2-(4-(4-hydroxyphenyl)piperazin-1-yl)-6-(indolin-1-yl)-9H-purin-9-yl)propanoate as a solid (130 mg, 60% yield).

Step 4: To a solution of methyl 3-(2-(4-(4-hydroxyphenyl)piperazin-1-yl)-6-(indolin-1-yl)-9H-purin-9-yl)propanoate (130 mg, 0.26 mmol, 1 eq) in THF (4 mL) at 0° C. under N2 atmosphere was added LAH (30 mg, 0.78 mmol, 3 eq). The mixture was stirred at room temperature for 1 hour before it was quenched with water (0.5 mL) and 1N aq. NaOH (0.5 mL). The solid was filtered off and rinsed with EtOAc. The filtrate was concentrated under reduced pressure. The crude residue was purified by Prep-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(9-(3-hydroxypropyl)-6-(indolin-1-yl)-9H-purin-2-yl)piperazin-1-yl)phenol as a white solid (40 mg, 300/% yield).

¹H NMR (300 MHz, DMSO-d6): δ 9.70 (br, 1H), 8.33 (d, J=7.8 Hz, 1H), 8.02 (s, 1H), 7.35-7.16 (m, 4H), 7.04-6.93 (m, 1H), 6.85-6.76 (m, 2H), 4.79-4.67 (m, 2H), 4.25-3.89 (m, 6H), 3.57-3.42 (m, 6H), 3.26-3.14 (m, 2H), 1.99-1.91 (m, 2H); LC/MS (ESI-MS): [M+H]+=472.2.

### Example 8. 3-(2-(4-(4-hydroxyphenyl)piperazin-1-yl)-6-(indolin-1-yl)-9H-purin-9-yl)propanoic acid

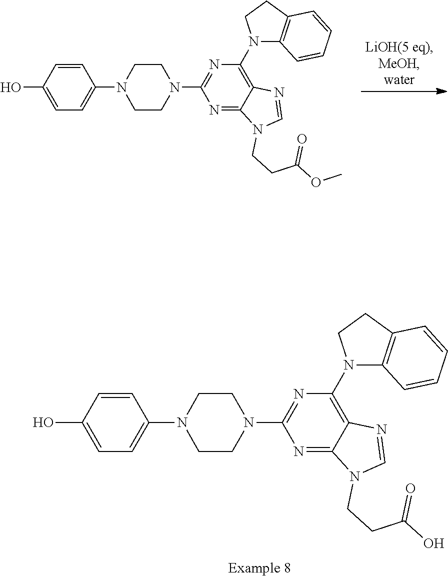 [SMILES: COC(=O)CCn1cnc2c(N3CCc4ccccc43)nc(N3CCN(c4ccc(O)cc4)CC3)nc21 ; O=C(O)CCn1cnc2c(N3CCc4ccccc43)nc(N3CCN(c4ccc(O)cc4)CC3)nc21]

To a solution of methyl 3-(2-(4-(4-hydroxyphenyl)piperazin-1-yl)-6-(indolin-1-yl)-9H-purin-9-yl)propanoate (130 mg, 0.26 mmol, 1 eq) in MeOH (4 mL) at 0° C. w as added LIOH (30 mg, 0.78 mmol, 3 eq). The mixture was stirred at room temperature for 1 hour before the organic solvent was removed under reduced pressure. The residue was purified by preparative-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 3-(2-(4-(4-hydroxyphenyl)piperazin-1-yl)-6-(indolin-1-yl)-9H-purin-9-yl)propanoic acid as the desired product in the TFA salt form (47 mg, 32% yield) as a white solid.

¹H NMR (400 MHz, DMSO-d6): δ 9.70 (br, 1H), 8.33 (d, J=8 Hz, 1H), 7.97 (s, 1H), 7.29-7.19 (m, 4H), 6.99-6.96 (m, 1H), 6.83-6.81 (m, 2H), 4.93-4.61 (m, 4H), 4.49-4.31 (m, 4H), 3.56-3.31 (m, 4H), 3.23 (t, J=8.4 Hz, 2H), 2.88 (t, J=6.8 Hz, 2H); LC/MS(ESI-MS): [M+H]+=486.2.

### Example 9. 4-(4-(6-(1H-benzo[d]imidazol-1-yl)-9-(2-hydroxyethyl)-9H-purin-2-yl)piperazin-1-yl)phenol

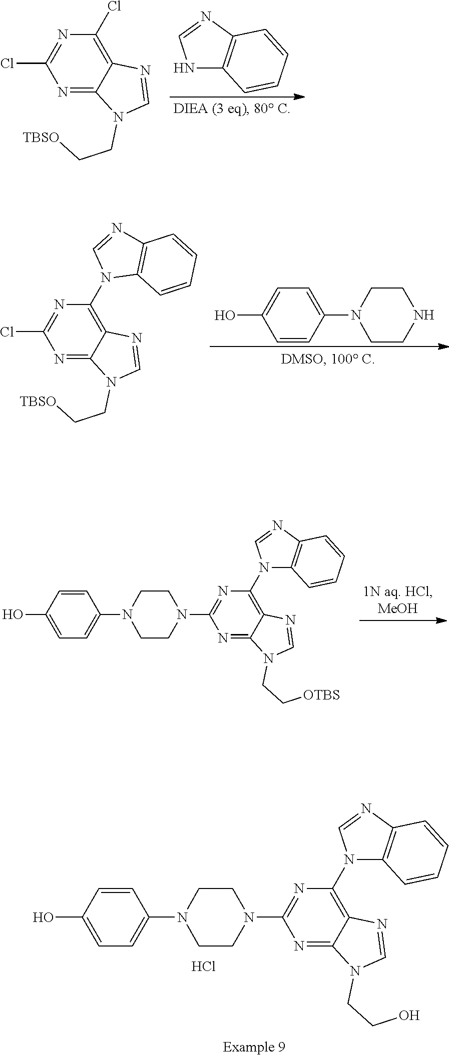

The proceeding example was prepared according to Example 1 substituting indoline with benzimidazole in the presence of DIEA. The desired product 4-(4-(6-(1H-benzo[d]-imidazol-1-yl)-9-(2-hydroxyethyl)-9H-purin-2-yl)piperazin-1-yl)phenol hydrochloride was isolated as a white solid.

¹H NMR (300 MHz, DMSO-d6): δ 9.86 (s, 1H), 8.55 (d, J=7.5 Hz, 1H), 8.37 (s, 1H), 7.84 (d, J=7.2 Hz, 1H), 7.76-7.71 (m, 2H), 7.50-7.44 (m, 3H), 6.93-6.88 (m, 3H), 4.27-4.21 (m, 4H), 3.88-3.71 (m, 8H); LC/MS (ESI-MS): [M+H]+=457.2.

### Example 10. 1-(9-(carboxymethyl)-2-(4-(4-hydroxyphenyl)piperazin-1-yl)-9H-purin-6-yl)-1H-indazole-3-carboxylic acid

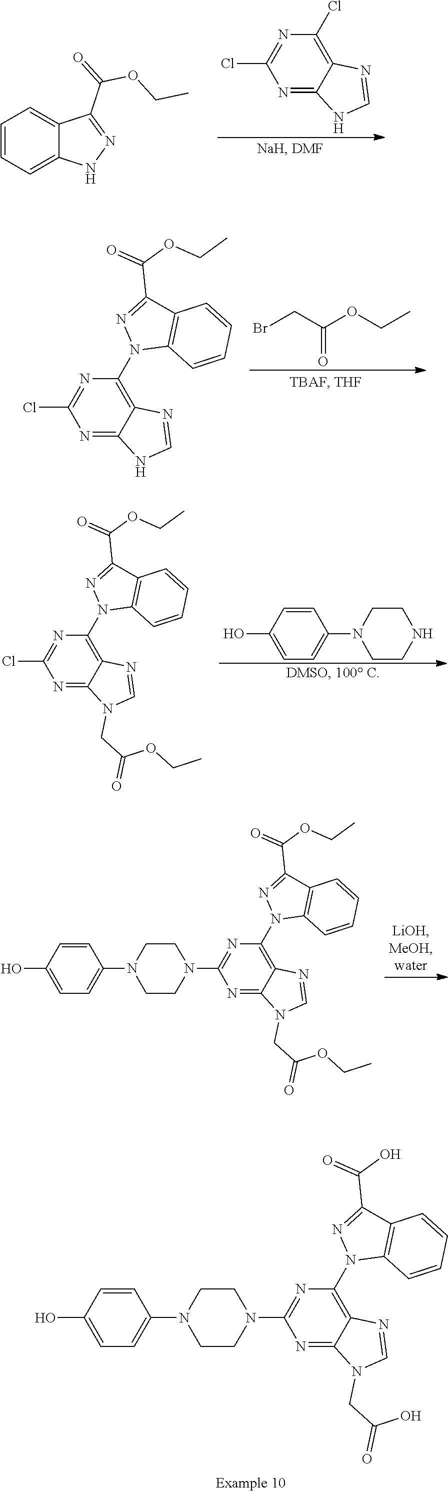

Step 1: To a solution of ethyl 1H-indazole-3-carboxylate (300 mg, 1.58 mmol, 1.0 eq) in DMF (5 mL) was added NaH (126 mg, 3.15 mmol, 2.0 eq, 60% in oil) in portions. The resulting mixture was stirred at room temperature for 30 min under a nitrogen atmosphere and followed by a solution of 2,6-dichloropurine (300 mg, 1.58 mmol, 1.0 eq) in DMF (2 mL). The resulting mixture was stirred at 80° C. overnight before it was allowed to cool and diluted with water (4 mL). The resulting solid precipitate was collected by filtration, wash with water (2 mL) and dried to provide ethyl 1-(2-chloro-9H-purin-6-yl)-1H-indazole-3-carboxylate (197 mg, 36% yield) as a solid.

Step 2: To a solution of ethyl 1-(2-chloro-9H-purin-6-yl)-1H-indazole-3-carboxylate (197 mg, 0.57 mmol, 1.0 eq) in THF (3 mL) under a nitrogen atmosphere at 0° C. was added a solution of TBAF (1.15 mL, 1.15 mmol, 1.0 M in TF, 2.0 eq) dropwise. After stirred at this temperature for 10 min, ethyl 2-bromoacetate (191.98 mg, 1.15 mmol, 2.0 eq) was added. The mixture was stirred at room temperature for 3 h before it was diluted with MeOH (3 mL). The resulting solid precipitate was collected by filtration, washed with water (2 mL) and dried to provide ethyl 1-(2-chloro-9-(2-ethoxy-2-oxoethyl)-9H-purin-6-yl)-1H-indazole-3-carboxylate as a solid (100 mg, 40% yield).

Step 3. A mixture of the product from the previous step (100 mg, 0.23 mmol, 1.0 eq) and 4-(piperazin-1-yl)phenol (124.7 mg, 0.70 mmol, 3.0 eq) in DMSO (3 mL) was stirred at 100° C. overnight under a nitrogen atmosphere. The reaction mixture was allowed to cool and diluted with water (6 mL). The resulting solid precipitate was collected by filtration, washed with water (2 mL) and dried to give the crude ethyl 1-(9-(2-ethoxy-2-oxoethyl)-2-(4-(4-hydroxyphenyl)piperazin-1-yl)-9H-purin-6-yl)-1H-indazole-3-carboxylate as a solid (163 mg) which was used in the next step without further purification.

Step 4: To a solution of crude ethyl 1-(9-(2-ethoxy-2-oxoethyl)-2-(4-(4-hydroxyphenyl)-piperazin-1-yl)-9H-purin-6-yl)-1H-indazole-3-carboxylate (163 mg, 0.29 mmol, 1.0 eq) in a mixture of H₂O (3 mL) and MeOH (3 mL) was added LiOH (120 mg, 28.6 mmol, 10.0 eq). The mixture was stirred at room temperature for 3 h before the solvent was removed under reduced pressure. The residue was re-dissolved in water (5 mL) and its pH was adjust to 5 with 2 N aq. HCl solution. The resulting precipitate was collected by filtration. The solid was further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 1-(9-(carboxymethyl)-2-(4-(4-hydroxyphenyl)piperazin-1-yl)-9H-purin-6-yl)-1H-indazole-3-carboxylic acid as a white solid (6.3 mg, 5% yield over two steps).

¹H NMR (400 MHz, DMSO-d6): 13.35 (br, 1H), 8.89 (s, 1H), 8.56 (d, J=8.0 Hz, 1H), 8.28-8.25 (m, 2H), 7.71-7.67 (m, 1H), 7.54-7.50 (m, 1H), 6.96-6.88 (m, 2H), 6.71-6.68 (m, 2H), 5.03 (s, 2H), 4.03-3.88 (m, 4H), 3.23-3.01 (m, 4H); LC/MS (ESI-MS): [M+H]+=515.1.

### Example 11. 2-(6-(3-(hydroxymethyl)-1H-indazol-1-yl)-2-(4-(4-hydroxyphenyl)piperazin-1-yl)-9H-purin-9-yl)acetic acid

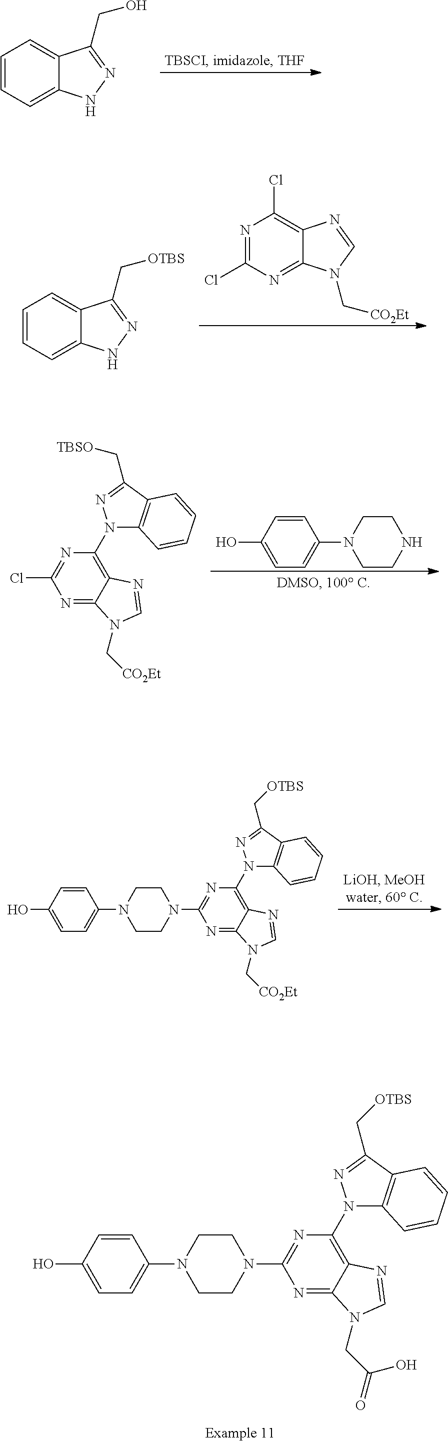

Step 1. To a solution of (1H-indazol-3-yl)methanol (1 g, 6.76 mmol, 1.0 eq) in THF (10 mL) at 0° C. was added imidazole (0.92 g, 13.5 mmol, 2.0 eq) and TBSCl (1.53 g, 3.97 mmol, 1.5 eq). The resulting mixture was stirred under a nitrogen atmosphere at room temperature overnight before it was diluted with water (15 mL) and extracted with EtOAc (4×15 mL). The combined organic layer was washed with brine, dried over Na₂SO₄ and concentration. The residue was purified by silica gel column chromatography (petroleum ether:EtOAc=7:3) to give 3-(((tert-butyldimethylsilyl)oxy)methyl)-1H-indazole as a white solid (1.28 g, 72% yield).

Step 2: To a solution of 3-(((tert-butyldimethylsilyl)oxy)methyl)-1H-indazole (1.26 g, 4.58 mmol, 1.0 eq) and ethyl 2-(2,6-dichloro-9H-purin-9-yl)acetate (1.2 g, 4.58 mmol, 1.0 eq) in DMF (8 mL) at room temperature was added K₂CO₃ (1.9 g, 13.74 mmol, 3.0 eq). The resulting mixture was stirred at 80° C. overnight under a nitrogen atmosphere before it was allowed to cool and diluted with water (20 mL) and extracted with EtOAc (3×20 mL). The combined organic layer was washed with brine, dried over Na₂SO₄ and concentration. The residue was purified by silica gel column chromatography (petroleum ether:EtOAc=2:1) to give ethyl 2-(6-(3-(((tert-butyldimethylsilyl)oxy)methyl)-1H-indazol-1-yl)-2-chloro-9H-purin-9-yl)acetate as a solid (380 mg, 16% yield).

Step 3: A mixture of the product from the previous step (150 mg, 0.3 mmol, 1.0 eq) and 4-(piperazin-1-yl)phenol (106.7 mg, 0.6 mmol, 2.0 eq) in DMSO (2 mL) was stirred at 100° C. overnight under a nitrogen atmosphere. The reaction was allowed to cool and diluted water (10 mL). The resulting solid was collected by filtration, washed with water (2 mL) and dried to provide crude ethyl 2-(6-(3-(hydroxymethyl)-1H-indazol-1-yl)-2-(4-(4-hydroxyphenyl)piperazin-1-yl)-9H-purin-9-yl)acetate as a solid (150 mg) which was used in the next step without further purification.

Step 4: To a solution of crude ethyl 2-(6-(3-(hydroxymethyl)-1H-indazol-1-yl)-2-(4-(4-hydroxyphenyl)piperazin-1-yl)-9H-purin-9-yl)acetate (150 mg, 0.28 mmol, 1.0 eq) in a mixture of H₂O (2 mL) and MeOH (2 mL) was added LiOH (59.54 mg, 1.42 mmol, 5.0 eq). The mixture was stirred at room temperature for 3 h before the solvent was removed under reduced pressure. The residue was diluted with water (5 mL) and its pH was adjusted to 3 with 2N aq. HCl. The resulting precipitate was collected by filtration and the solid was further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(6-(3-(hydroxymethyl)-1H-indazol-1-yl)-2-(4-(4-hydroxyphenyl)piperazin-1-yl)-9H-purin-9-yl)acetic acid as a white solid (32 mg, 21% yield over two steps).

¹H NMR (300 MHz, DMSO-d6): δ 8.59-8.51 (m, 2H), 8.04 (d, J=8.1 Hz, 1H), 7.66-7.60 (m, 1H), 7.43-7.37 (m, 1H), 7.16-7.05 (m, 2H), 6.74 (d, J=8.1 Hz, 1H), 5.03 (s, 2H), 4.93 (s, 2H), 4.03-3.93 (m, 4H), 3.33-3.26 (m, 4H); LC/MS (ESI-MS): [M+H]+=501.2.

### Example 12. 4-(4-(6-(1H-indazol-1-yl)-9-(pyridin-3-yl)-9H-purin-2-yl)piperazin-1-yl)phenol

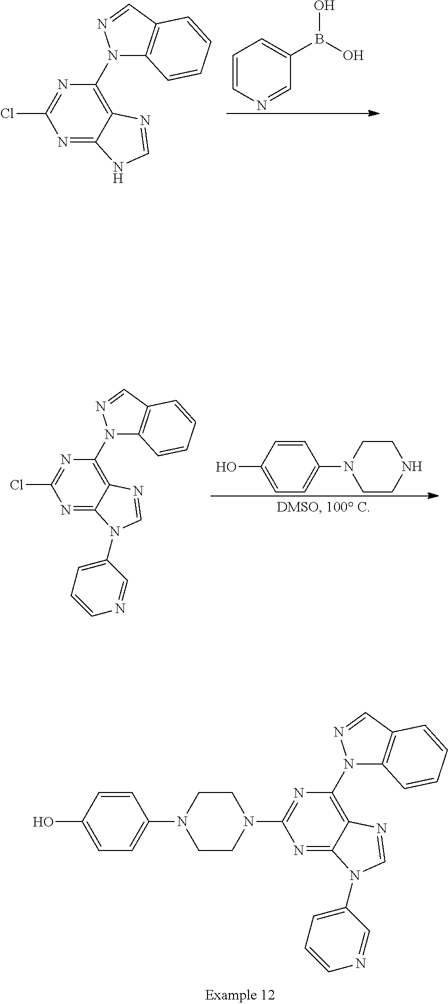

### Step 1:

To a solution of 2-chloro-6-(1H-indazol-1-yl)-9H-purine (0.5 g, 1.85 mmol, 1.0 eq) in DMF (10 mL) was added pyridin-3-ylboronic acid (0.68 g, 5.56 mmol, 3 eq), 1,10-phenanthroline monohydrate (0.74 g, 3.70 mmol, 2 eq), anhydrous cupric acetate (0.67 g, 3.70 mmol, 2 eq). The reaction mixture was then stirred for 3 days at 25° C. before it was diluted with water (20 mL). The precipitate was collected by filtration to give the crude 2-chloro-6-(1H-indazol-1-yl)-9-(pyridin-3-yl)-9H-purine (0.1 g) as a gray solid which was used in next step without further purification.

### Step 2:

To a solution of crude 2-chloro-6-(1H-indazol-1-yl)-9-(pyridin-3-yl)-9H-purine (0.1 g, 0.29 mmol, 1.0 eq) in DMSO (5 mL) was added 1-(4-hydroxyphenyl)piperazine (0.15 g, 0.86 mmol, 3.0 eq). The mixture was stirred overnight at 100° C. under a nitrogen atmosphere before it was diluted with water (10 mL) to produce a precipitate. The solid was collected by filtration and purified by prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(6-(1H-indazol-1-yl)-9-(pyridin-3-yl)-9H-purin-2-yl)piperazin-1-yl)phenol (5 mg) as a white solid.

¹H NMR (400 MHz, CD₃OD-d4): δ 8.69 (s, 1H), 8.57 (s, 1H), 8.56-8.45 (m, 2H), 7.83 (d, J=7.6 Hz, 1H), 7.65 (d, J=7.6 Hz, 1H), 7.68 (s, 1H), 7.59 (t, J=7.6 Hz, 1H), 7.48-7.45 (m, 3H), 7.34 (t, J=7.6 Hz, 1H), 6.94 (d, J=7.2 Hz, 2H), 4.47-4.21 (m, 4H), 3.77-3.72 (m, 4H); LC/MS (ESI-MS): [M+H]+=490.1.

### Example 13. 4-(4-(3-bromo-7-(1H-indazol-1-yl)pyrazolo[1,5-a]pyrimidin-5-yl)piperazin-1-yl)phenol

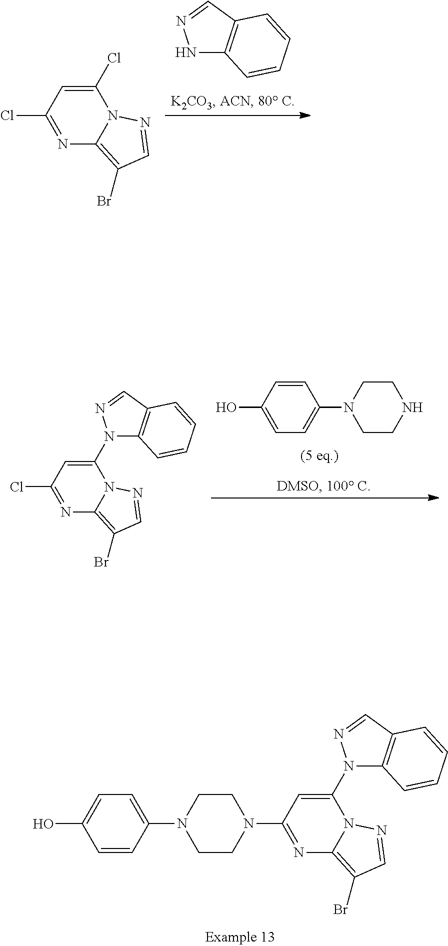

### Step 1:

A mixture of 3-bromo-5,7-dichloropyrazolo[1,5-a]pyrimidine (1 g, 3.76 mmol, 1.0 eq), indazole (0.44 g, 3.76 mmol, 1.0 eq) and K₂CO₃ (1.56 g, 11.28 mmol, 3 eq) in MeCN (20 mL) was stirred overnight at 80° C. The reaction mixture was allowed to cool and diluted with water (20 mL) and extracted with EtOAc (50 mL). The organic layer was washed with brine, dried over Na₂SO₄, and concentrated. The residue was purified by flash column chromatography (petroleum ether:EtOAc=10:1) to give 3-bromo-5-chloro-7-(1H-indazol-1-yl)pyrazolo[1,5-a]pyrimidine (0.25 g, 17% yield) as a white solid.

### Step 2:

A mixture of 3-bromo-5-chloro-7-(1H-indazol-1-yl)pyrazolo[1,5-a]pyrimidine (0.25 g, 0.72 mmol, 1.0 eq) and 1-(4-hydroxyphenyl) piperazine (0.38 g, 2.16 mmol, 3.0 eq) in DMSO (5 mL) under a nitrogen atmosphere was stirred overnight at 100° C. The reaction was allowed to cool and diluted with water (10 mL). The resulting solid was collected by filtration and further purified by Prep-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(3-bromo-7-(1H-indazol-1-yl)pyrazolo[1,5-a]pyrimidin-5-yl)piperazin-1-yl)phenol (81 mg, 25% yield) as a solid.

¹H NMR (300 MHz, DMSO-d6): δ 8.90 (s, 1H), 8.62 (s, 1H), 8.05 (s, 1H), 7.96 (d, J=7.8 Hz, 1H), 7.53-7.48 (m, 2H), 7.38-7.30 (m, 1H), 7.18 (s, 1H), 6.88 (d, J=8.7 Hz, 2H), 6.70 (d, J=9 Hz, 2H), 3.99-3.89 (m, 4H), 3.15-3.04 (m, 4H); LC/MS(ESI-MS): [M+H]+=490.0.

### Example 14. 4-(4-(7-(1H-indazol-1-yl)-3-(pyridin-3-yl)pyrazolo[1,5-a]pyrimidin-5-yl)piperazin-1-yl)phenol

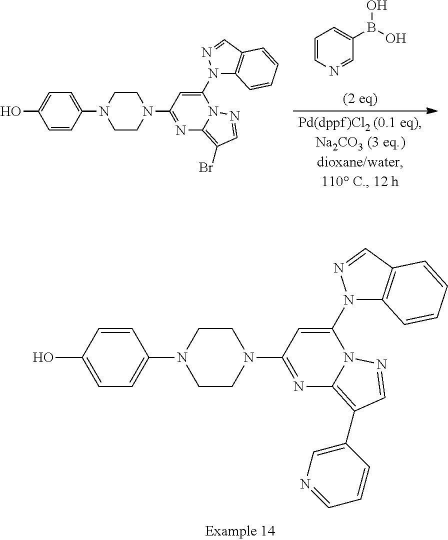 [SMILES: Oc1ccc(N2CCN(c3cc(-n4ncc5ccccc54)n4ncc(Br)c4n3)CC2)cc1 ; OB(O)c1cccnc1 ; Oc1ccc(N2CCN(c3cc(-n4ncc5ccccc54)n4ncc(-c5cccnc5)c4n3)CC2)cc1]

To a solution of 4-(4-(3-bromo-7-(1H-indazol-1-yl)pyrazolo[1,5-a]pyrimidin-5-yl)piperazin-1-yl)phenol (70 mg, 0.14 mmol, 1.0 eq) in a mixture of dioxane (2 mL) and water (0.5 mL) was added 3-pyridineboronic acid (26 mg, 0.21 mmol, 1.5 eq), Na₂CO₃ (45 mg, 0.43 mmol, 3 eq), Pd(dppf)Cl2 (23 mg, 0.02 mmol, 0.1 eq). The mixture was stirred at 100° C. for 24 h before it was concentrated under reduced pressure. The residue was partitioned between water (10 mL) and EtOAc (30 mL). The organic layer was washed with brine, dried over Na₂SO₄, concentrated. The residue was purified by prep-HPLC to give 4-(4-(7-(1H-indazol-1-yl)-3-(pyridin-3-yl)pyrazolo[1,5-a]pyrimidin-5-yl)piperazin-1-yl)phenol (7 mg, 10% yield) as a white solid.

¹H NMR (400 MHz, DMSO-d6): δ 9.47 (s, 1H), 8.94 (br s, 2H), 8.70 (s, 1H), 8.64 (s, 1H), 8.58 (d, J=4.8 Hz, 1H), 7.97 (d, J=8.1 Hz, 1H), 7.86 (br s, 1H), 7.53 (s, 2H), 7.41-7.33 (m, 1H), 7.25 (s, 1H), 6.93 (d, J=8.7 Hz, 2H), 6.71 (d, J=8.7 Hz, 2H), 4.08-3.98 (m, 4H), 3.24-3.12 (m, 4H); LC/MS(ESI-MS): [M+H]+=489.2.

### Example 15. (S)-2-(2-(4-(4-hydroxyphenyl)-2-methylpiperazin-1-yl)-6-(1H-indazol-1-yl)-9H-purin-9-yl)acetic acid

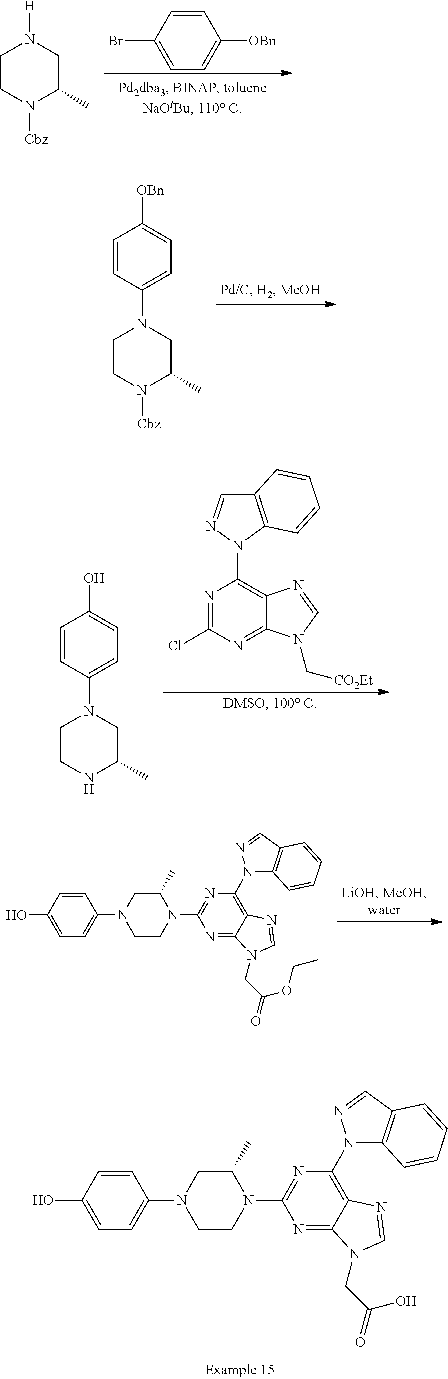

### Step 1:

To a solution of (S)-benzyl 2-methylpiperazine-1-carboxylate (0.4 g, 1.71 mmol, 1.0 eq) in toluene (10 mL) was added 4-benzyloxybromobenzene (0.45 g, 1.71 mmol, 1.0 eq), Pd2dba3 (0.23 g, 0.26 mmol, 0.15 eq), BINAP (0.32 g, 0.51 mmol, 0.3 eq) and sodium tert-butoxide (0.41 g, 4.27 mmol, 2.5 eq). The mixture was stirred overnight at 110° C. under a nitrogen atmosphere before it was allowed to cool to 25° C. The mixture was diluted with water (50 ml) and extracted with EtOAc (2×50 mL). The combined organic layer was washed with brine, dried over Na₂SO₄ and concentrated. The residue was purified by flash column chromatography on silica gel (petroleum ether:EtOAc=5:1) to give (S)-benzyl 4-(4-(benzyloxy)phenyl)-2-methylpiperazine-1-carboxylate (0.43 g, 60% yield).

### Step 2:

A mixture of (S)-benzyl 4-(4-(benzyloxy)phenyl)-2-methylpiperazine-1-carboxylate (0.43 g, 1.03 mmol, 1.0 eq) and Pd/C catalyst (0.17 g, 40% w/w, 10% Pd on carbon) in methanol (10 mL) at 25° C. under a H2 atmosphere (1 atm) was stirred overnight before the catalyst was filtered off. The filtrate was concentrated to give (S)-4-(3-methylpiperazin-1-yl)phenol (0.22 g of, 80% yield).

### Step 3:

To a mixture of (S)-4-(3-methylpiperazin-1-yl)phenol (0.2 g, 1.0 mmol, 2.5 eq) and in (6 mL) DMSO under N2 atmosphere was added ethyl 2-(2-chloro-6-(1H-indazol-1-yl)-9H-purin-9-yl)acetate (0.14 g, 0.4 mmol, 1.0 eq). Then the mixture was stirred overnight at 100° C. before it was allowed to cool to 25° C. and diluted with water (50 mL). The solid was collected by filtration and dried to give (S)-ethyl 2-(2-(4-(4-hydroxyphenyl)-2-methyl-piperazin-1-yl)-6-(1H-indazol-1-yl)-9H-purin-9-yl)acetate (0.3 g, 58% yield) as an off-white solid.

### Step 4:

To a solution of (S)-ethyl 2-(2-(4-(4-hydroxyphenyl)-2-methylpiperazin-1-yl)-6-(1H-indazol-1-yl)-9H-purin-9-yl)acetate (0.3 g, 0.59 mmol, 1.0 eq) in MeOH (10 mL) and water (5 mL) was added LiOH (0.12 g, 2.93 mmol, 5 eq). The mixture was stirred for 0.5 hours at room temperature before it was concentrated. The residue was diluted with water (10 mL) and adjusted its pH to 3 with 5N aq. HCl solution. The precipitate was collected by filtration and purified by preparative-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give (S)-2-(2-(4-(4-hydroxyphenyl)-2-methylpiperazin-1-yl)-6-(1H-indazol-1-yl)-9H-purin-9-yl)acetic acid (15 mg) as white solid.

¹H NMR (400 MHz, CD3OD-d4): δ 9.59 (s, 1H), 8.59 (s, 1H), 7.89-7.73 (m, 2H), 7.43-7.34 (m, 3H), 7.21-7.19 (m, 1H), 6.92 (d, J=8.4 Hz, 2H), 10.06 (br, 1H), 5.45-5.34 (m, 1H), 5.14-5.01 (m, 3H), 3.73-3.50 (m, 5H), 1.56 (d, J=6.4 Hz, 2H); LC/MS(ESI-MS): [M+H]+=485.2.

### Example 16. 2-(6-(4-(hydroxymethyl)-1H-indazol-1-yl)-2-(4-(4-hydroxyphenyl)piperazin-1-yl)-9H-purin-9-yl)acetic acid

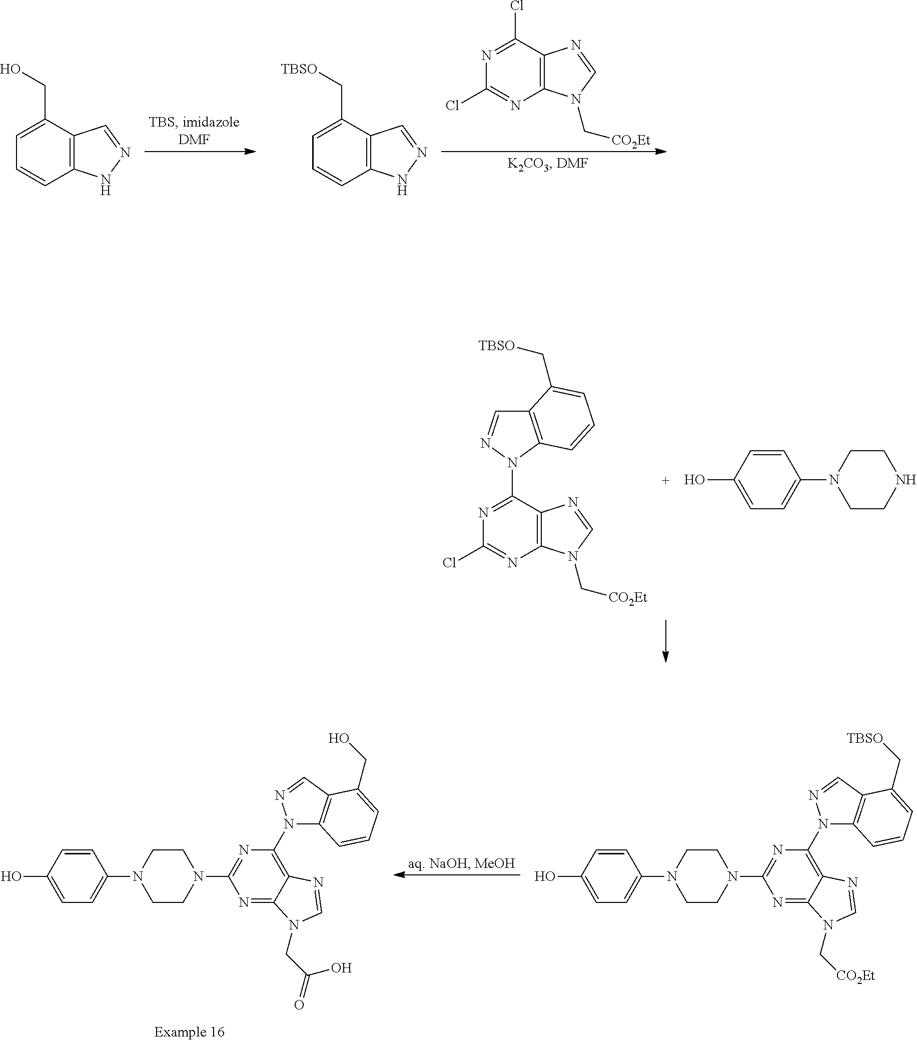

### Step 1:

To a mixture of (1H-indazol-4-yl)methanol (200 mg, 1.4 mmol, 1.0 eq) in THF (5 mL) at 25° C. under a N2 atmosphere was added imidazole (110 mg, 1.6 mmol, 1.2 eq) and TBSCl (213 Mg, 1.47 mmol, 1.05 eq). After stirring for overnight the solution was evaporated to afford the crude product. The crude product was purified by flash column chromatography on silica gel (petroleum ether:EtOAc=5:1) to give (260 mg, 73% yield) 4-(((tert-butyldimethyl-silyl)oxy)methyl)-1H-indazole as a colorless oil.

### Step 2:

A mixture of 4-(((tert-butyldimethyl-silyl)oxy)methyl)-1H-indazole (130 mg, 0.5 mmol, 1.0 eq), ethyl 2-(2,6-dichloro-9H-purin-9-yl)acetate (138 mg, 0.5 mmol, 1.0 eq) and K₂CO₃ (210 mg, 1.5 mmol, 3 eq) in DMSO (5 mL) was stirred for overnight at 50° C. The reaction was allowed to cool to 25° C. and diluted with water (20 mL) and extracted with EtOAc (2×10 mL). The combined organic layer was washed with water, brine, dried over Na₂SO₄, concentrated. The residue was purified by flash column chromatography on silica gel (petroleum ether:EtOAc=1:1) to give ethyl 2-(6-(4-(((tert-butyldimethylsilyl)oxy)methyl)-1H-indazol-1-yl)-2-chloro-9H-purin-9-yl)acetate (100 mg, 20% yield) as an off-white solid.

### Step 3:

A mixture of ethyl 2-(6-(4-(((tert-butyldimethylsilyl)oxy)methyl)-1H-indazol-1-yl)-2-chloro-9H-purin-9-yl)acetate (50 mg, 0.1 mmol, 1.0 eq) and 4-(piperazin-1-yl)phenol (53 mg, 0.3 mmol, 3 eq) in (5 mL) DMSO was stirred at 100° C. under N2 atmosphere overnight. The reaction was allowed to cool to 25° C. and diluted with water (10 mL). The precipitate was collected by filtration and dried to give ethyl 2-(6-(4-(((tert-butyldimethylsilyl)oxy)methyl)-1H-indazol-1-yl)-2-(4-(4-hydroxyphenyl)piperazin-1-yl)-9H-purin-9-yl)acetate (60 mg, 94% yield) as a brown solid.

### Step 4:

A mixture of ethyl 2-(6-(4-(((tert-butyldimethylsilyl)oxy)methyl)-1H-indazol-1-yl)-2-(4-(4-hydroxyphenyl)piperazin-1-yl)-9H-purin-9-yl)acetate (60 mg, 0.09 mmol, 1.0 eq) and 1N aq. NaOH solution (5 mL) in MeOH (15 mL) was stirred for 2 hours at 50° C. The reaction was allowed to cool and concentrated under reduced pressure. The residue was re-dissolved in water (2 mL) and acidified to pH 3 with 2 N aq. HCl. The mixture was extracted with EtOAc (2×10 mL). The combined organic layer was washed with water, brine, dried over Na₂SO₄, concentrated. The residue was purified by Prep-HPLC (Column: Waters SunFire Prep C18 OBD 5 um 19*150 mm Column; Mobile phase A. Acetonitrile; Mobile phase B: 0.1% TFA in Water; Flow rate: 20.0 ml/min; Detection UV: 210 nm) to give 2-(6-(4-(hydroxymethyl)-1H-indazol-1-yl)-2-(4-(4-hydroxyphenyl)piperazin-1-yl)-9H-purin-9-yl)acetic acid (4 mg) as a white solid.

¹H NMR (400 MHz, DMSO-d6): δ 9.12 (br, 1H), 8.81-8.65 (m, 1H), 8.54-8.47 (m, 1H), 8.39-8.28 (m, 1H), 7.67-7.54 (m, 1H), 7.42-7.31 (m, 1H), 7.08-6.93 (m, 2H), 6.79-6.65 (m, 2H), 5.02 (s, 2H), 4.93 (s, 2H), 4.18-3.95 (m, 4H), 2.83-2.72 (m, 4H); LC/MS(ESI-MS): [M+H]+=501.2.

### Example 17. 4-(4-(9-((1H-tetrazol-5-yl)methyl)-6-(1H-indazol-1-yl)-9H-purin-2-yl)piperazin-1-yl)phenol

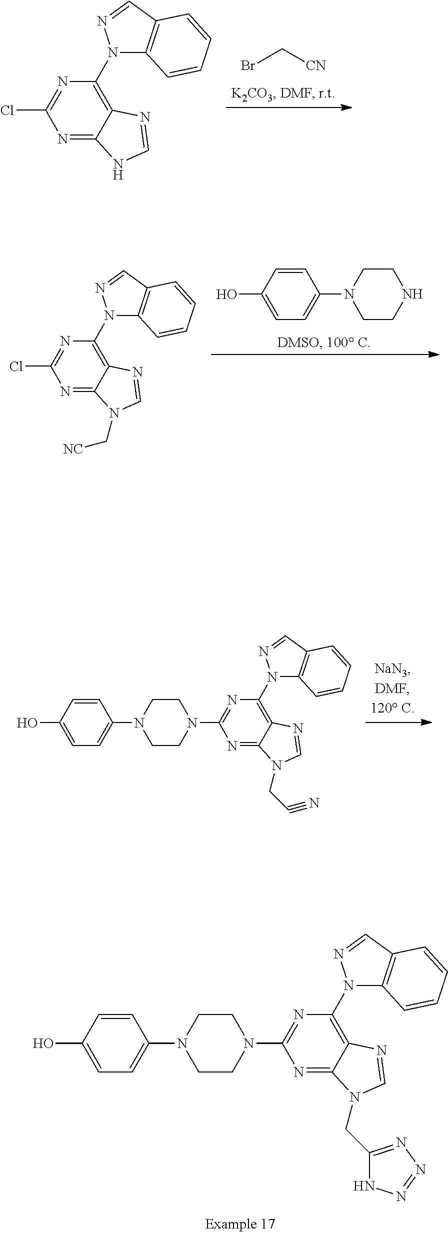

### Step 1:

To a solution of 2-chloro-6-(1H-indazol-1-yl)-9H-purine (0.5 g, 1.85 mmol, 1.0 eq) in DMF (10 mL) at 25° C. was added bromoacetonitrile (0.33 g, 2.78 mmol, 1.5 eq) and K₂CO₃ (0.51 g, 3.70 mmol, 2 eq). The mixture was stirred under a nitrogen atmosphere overnight before it was diluted with water (60 mL). The solid was collected by filtration then purified by Prep-HPLC to give 2-(2-chloro-6-(1H-indazol-1-yl)-9H-purin-9-yl)acetonitrile (0.18 g, 32% yield).

### Step 2:

A mixture of 2-(2-chloro-6-(1H-indazol-1-yl)-9H-purin-9-yl)acetonitrile (0.18 g, 0.58 mmol, 1.0 eq) and 1-(4-hydroxyphenyl) piperazine (0.31 g, 1.74 mmol, 3.0 eq) in DMSO (5 mL) was stirred overnight at 100° C. The reaction was allowed to cool and diluted with water (10 mL). The solid was collected by filtration and dried to give 2-(2-(4-(4-hydroxyphenyl)-piperazin-1-yl)-6-(1H-indazol-1-yl)-9H-purin-9-yl)acetonitrile (0.14 g, 58% yield) as an off-white solid.

### Step 3:

A mixture of 2-(2-(4-(4-hydroxyphenyl)-piperazin-1-yl)-6-(1H-indazol-1-yl)-9H-purin-9-yl)acetonitrile (0.14 g, 0.30 mmol, 1 eq), NH4Cl (28 mg, 0.53 mmol, 2.0 eq) and NaN3 (26 mg, 0.40 mmol, 1.5 eq) in DMF (5 mL) was stirred overnight at 110° C. The reaction was allowed to cool and diluted with water (10 mL). The solid was collected by filtration and purified by Prep-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(9-((1H-tetrazol-5-yl)methyl)-6-(1H-indazol-1-yl)-9H-purin-2-yl)piperazin-1-yl)phenol (10 mg, 8% yield) as an off-white solid.

¹H NMR (300 MHz, DMSO-d6): δ 9.31 (br, 1H), 8.61-8.42 (m, 2H), 8.01-7.93 (m, 1H), 7.91-7.81 (m, 1H), 7.49-7.41 (m, 1H), 7.11-6.96 (m, 3H), 6.75-6.72 (m, 3H), 5.92-5.86 (m, 2H), 4.07-3.89 (m, 4H), 3.29-3.11 (m, 4H); LC/MS(ESI-MS): [M+H]+=495.1.

### Examples 18 and 19. 2-(6-(6-chloro-1H-benzo[d][1,2,3]triazol-1-yl)-2-(4-(4-hydroxyphenyl)piperazin-1-yl)-9H-purin-9-yl)acetic acid and 2-(6-(5-chloro-1H-benzo[d][1,2,3]triazol-1-yl)-2-(4-(4-hydroxyphenyl)piperazin-1-yl)-9H-purin-9-yl)acetic acid

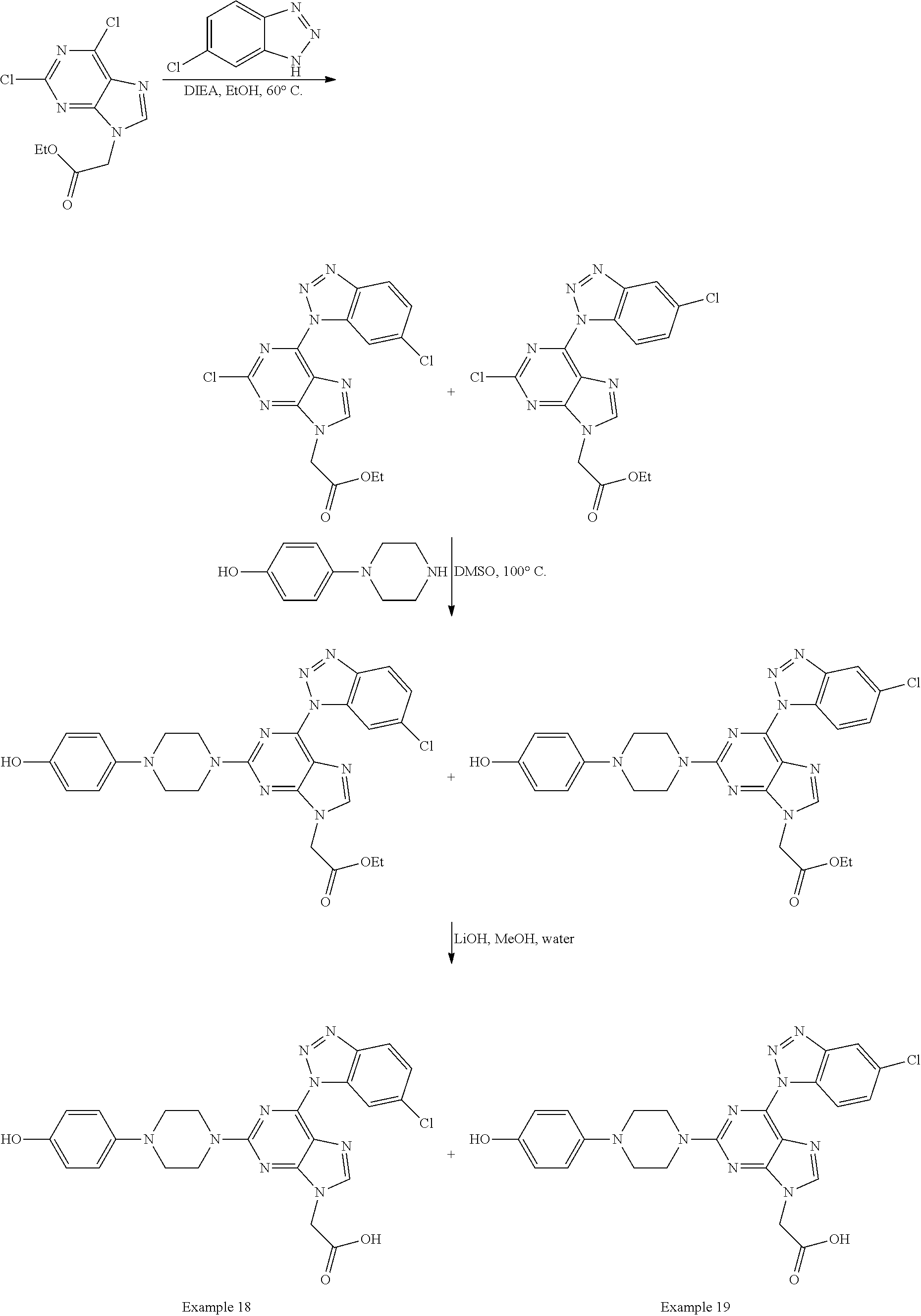

### Step 1:

To the solution of ethyl 2-(2,6-dichloro-9H-purin-9-yl)acetate (300 mg, 1.09 mmol, 1.0 eq) and 6-chloro-1H-benzo[d][1,2,3]triazole (167 mg, 1.09 mmol, 1.0 eq) in EtOH (5 mL) was added DIEA (423 mg, 3.27 mmol, 3.0 eq). The mixture was stirred at 60° C. overnight under a nitrogen atmosphere before it was allowed to cool. The precipitate was collected by filtration and rinsed with EtOH and dried to provide a mixture of ethyl 2-(2-chloro-6-(6-chloro-1H-benzo[d][1,2,3]triazol-1-yl)-9H-purin-9-yl)acetate and ethyl 2-(2-chloro-6-(5-chloro-1H-benzo[d][1,2,3]triazol-1-yl)-9H-purin-9-yl)acetate (320 mg) as a white solid which was used directly in next step without further purification.

### Step 2:

A mixture of ethyl 2-(2-chloro-6-(6-chloro-1H-benzo[d][1,2,3]triazol-1-yl)-9H-purin-9-yl)acetate and ethyl 2-(2-chloro-6-(5-chloro-1H-benzo[d][1,2,3]triazol-1-yl)-9H-purin-9-yl)acetate (200 mg, 0.51 mmol, 1 eq) and 1-(4-hydroxyphenyl) piperazine (182 mg, 1.02 mmol, 2 eq) in DMSO (2 mL) under a nitrogen atmosphere was stirred at 100° C. overnight. The reaction was allowed to cool and diluted with water (8 mL). The resulting solid was collected by filtration and rinsed with MeOH (1 mL), and dried to provide a mixture of ethyl 2-(6-(6-chloro-1H-benzo[d][1,2,3]triazol-1-yl)-2-(4-(4-hydroxyphenyl)piperazin-1-yl)-9H-purin-9-yl)acetate and ethyl 2-(6-(5-chloro-1H-benzo[d][1,2,3]triazol-1-yl)-2-(4-(4-hydroxyphenyl)piperazin-1-yl)-9H-purin-9-yl)acetate (320 mg) as a brown solid which was used in next step without further purification.

### Step 3:

To a solution of mixture of ethyl 2-(6-(6-chloro-1H-benzo[d][1,2,3]triazol-1-yl)-2-(4-(4-hydroxyphenyl)piperazin-1-yl)-9H-purin-9-yl)acetate and ethyl 2-(6-(5-chloro-1H-benzo-[d][1,2,3]triazol-1-yl)-2-(4-(4-hydroxyphenyl)piperazin-1-yl)-9H-purin-9-yl)acetate (200 mg, 0.51 mmol, 1.0 eq) in MeOH (2 mL) and water (2 mL) was added LiOH (58.93 mg, 1.40 mmol, 5.0 eq). The resulting mixture was stirred at room temperature for 3 hours before it was concentrated. The residue was re-dissolved in water (2 mL) and adjusted its pH to 4 with 2N aq. HCl solution. The precipitate was collected by filtration and further purified by Prep-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(6-(6-chloro-1H-benzo[d][1,2,3]triazol-1-yl)-2-(4-(4-hydroxyphenyl)-piperazin-1-yl)-9H-purin-9-yl)acetic acid (12.6 mg) and 2-(6-(5-chloro-1H-benzo[d][1,2,3]-triazol-1-yl)-2-(4-(4-hydroxyphenyl)piperazin-1-yl)-9H-purin-9-yl)acetic acid (4.9 mg) both as white solid.

### Data of Example 18:

¹H NMR (400 MHz, DMSO-d6): δ 8.47-8.45 (m, 2H), 8.37 (s, 1H), 7.82-7.80 (m, 1H), 7.15-7.01 (m, 2H), 6.77-6.75 (m, 2H), 5.06 (s, 2H), 4.19-3.91 (m, 4H), 3.35-3.21 (m, 4H); LC/MS(ESI-MS): [M+H]+=506.2.

### Data of Example 19.

¹H NMR (400 MHz, DMSO-d6): δ 8.89 (s, 1H), 8.49 (s, 1H), 8.34-8.32 (m, 2H), 7.68-7.65 (m, 1H), 6.89 (d, J=8.8 Hz, 2H), 6.69 (d, J=8.8 Hz, 2H), 4.98 (s, 2H), 4.05-3.95 (m, 4H), 3.13-3.07 (m, 4H); LC/MS(ESI-MS): [M+H]+=501.2.

### Example 20. 2-(2-(4-(4-hydroxyphenyl)piperazin-1-yl)-6-(4-methyl-1H-benzo[d][1,2,3]triazol-1-yl)-9H-purin-9-yl)acetic acid

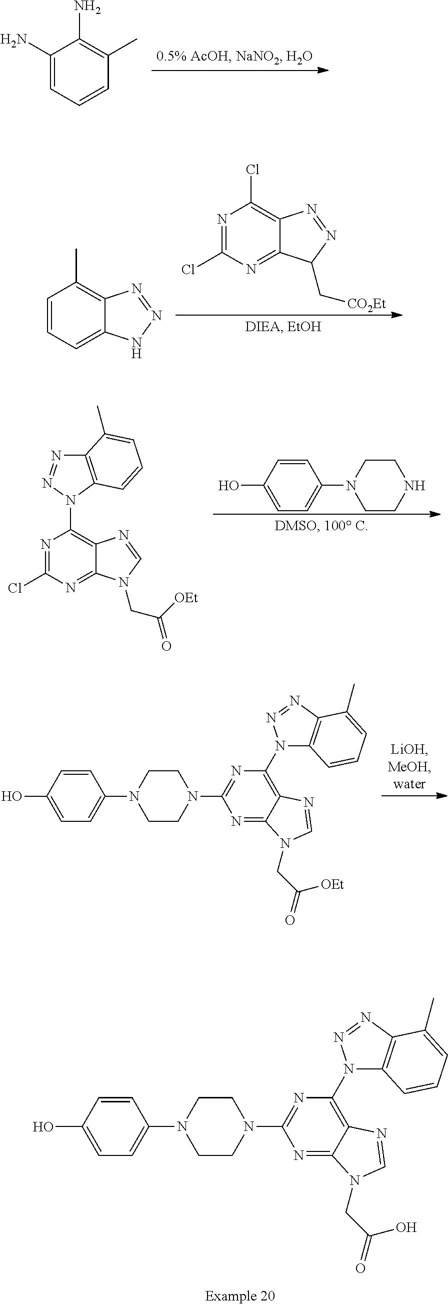

### Step 1:

To a solution of 3-methylbenzene-1,2-diamine (2 g, 16.4 mmol, 1.0 eq) in 0.5% aq. AcOH (70 mL) at 0-4° C. was added a solution of NaNO2 (3 g, 24.6 mmol, 1.5 eq) in water (50 mL). The mixture was stirred at room temperature for 4 hours before it was extracted with EtOAc (4×50 mL). The combined organic layer was washed with water, brine and concentrated. The crude residue was purified by flash column chromatography (petroleum ether:EtOAc=1:1) to provide 4-methyl-1H-benzo[d][1,2,3]triazole (600 mg, 28% yield) as an orange solid.

### Step 2:

To the solution of 4-methyl-H-benzo[d][1,2,3]triazole (300 mg, 1.09 mmol, 1.0 eq) and ethyl 2-(2,6-dichloro-9H-purin-9-yl)acetate (145 mg, 1.09 mmol, 1.0 eq) in EtOH (5 mL) was added DIEA (423 mg, 3.27 mmol, 3.0 eq). The mixture was stirred at 60° C. overnight under a nitrogen atmosphere before it was concentrated under reduced pressure. The resulted solid slurried with EtOH and the precipitate was collected by filtration and dried to provide crude ethyl 2-(2-chloro-6-(4-methyl-1H-benzo[d][1,2,3]triazol-1-yl)-9H-purin-9-yl)acetate (270 mg) as a white solid which was used directly in next step.

### Step 3:

The solution of crude ethyl 2-(2-chloro-6-(4-methyl-1H-benzo[d][1,2,3]triazol-1-yl)-9H-purin-9-yl)acetate (270 mg, 0.73 mmol, 1.0 eq), 4-(piperazin-1-yl)phenol (259 mg, 1.45 mmol, 2.0 eq) in DMSO (3 mL) was stirred at 100° C. overnight under nitrogen atmosphere. The mixture was allowed cool to room temperature and diluted with water (6 mL). The resulting solid was collected by filtration and washed with MeOH (1 mL) and dried to provide the crude ethyl 2-(2-(4-(4-hydroxyphenyl)piperazin-1-yl)-6-(4-methyl-1H-benzo[d]-[1,2,3]triazol-1-yl)-9H-purin-9-yl)acetate (430 mg) as a solid which was used in next step without further purification.

### Step 4:

To a solution of crude ethyl 2-(2-(4-(4-hydroxyphenyl)piperazin-1-yl)-6-(4-methyl-1H-benzo[d]-[1,2,3]triazol-1-yl)-9H-purin-9-yl)acetate (200 mg, 0.39 mmol, 1.0 eq) in MeOH (2 mL) and water (2 mL) was added LIOH (82 mg, 1.95 mmol, 5.0 eq). The resulting mixture was stirred at room temperature for 3 hours before the organic volatile was removed under reduced pressure. The aq. solution was acidified to pH 4 with 2N aq. HCl and the precipitate was collected by filtration. The crude product was further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(4-methyl-1H-benzo[d][1,2,3]triazol-1-yl)-9H-purin-9-yl)acetic acid (7.2 mg, 4% yield) as pale solid.

¹H NMR (300 MHz, DMSO-d6): δ 8.31 (s, 1H), 8.22-8.19 (m, 1H), 7.60 (t, J=7.2 Hz, 1H), 7.36-7.33 (m, 1H), 7.20-7.05 (m, 2H), 6.74-6.72 (m, 2H), 5.01 (s, 2H), 4.14-3.98 (m, 4H), 3.39-3.23 (m, 4H), 2.77 (s, 3H); LCMS (ESI-MS): [M+H]+=486.2.

### Example 21. 2-(2-(4-(5-hydroxypyridin-2-yl)piperazin-1-yl)-6-(1H-indazol-1-yl)-9H-purin-9-yl)acetic acid

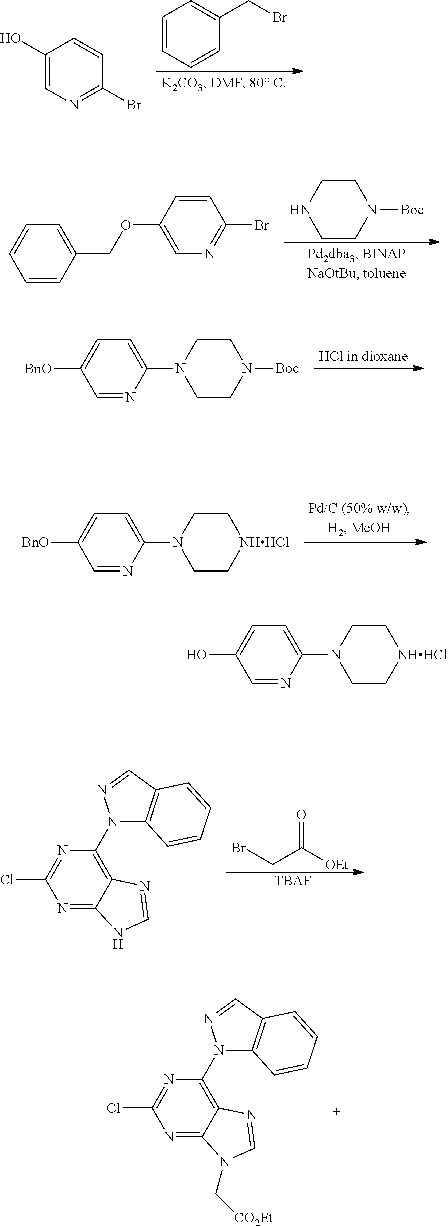

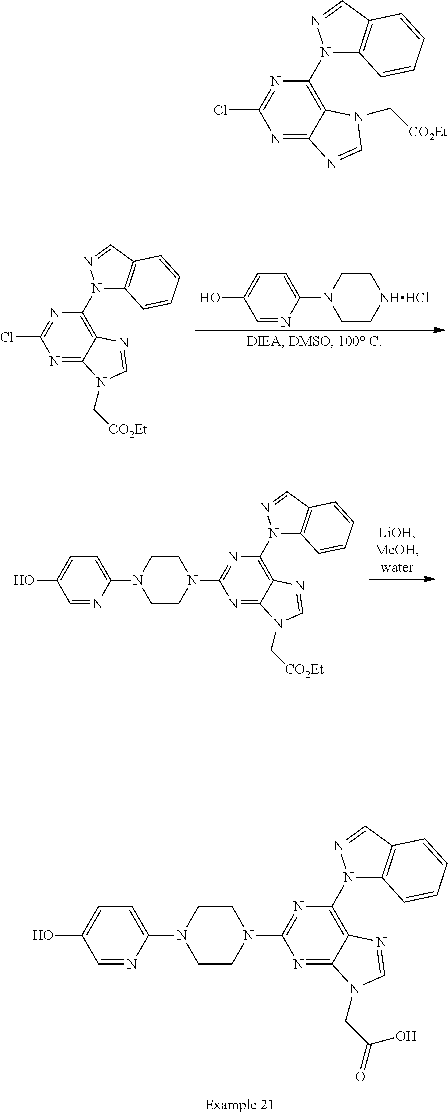

### Step 1:

To the solution of 6-bromopyridin-3-ol (1 g, 5.75 mmol, 1.0 eq) and K₂CO₃ (2.38 g, 17.24 mmol, 3.0 eq) in DMF (10 mL) was added benzyl bromide (1.47 g, 8.62 mmol, 1.5 eq). The mixture was stirred at 60° C. for 4 hours under a nitrogen atmosphere before it was allowed to cool to room temperature and diluted with water (400 mL) and extracted with EtOAc (3×200 mL). The combined organic layer was washed with water (100 mL) and brine (50 mL), and concentrated to provide crude 5-(benzyloxy)-2-bromopyridine (1.59 g) as a white solid which was used in next step without further purification.

### Step 2:

To a three-neck bottle under a nitrogen atmosphere was charged a mixture of 5-(benzyloxy)-2-bromopyridine (1.58 g, 5.98 mmol, 1.0 eq), tert-butyl piperazine-1-carboxylate (1.23 g, 6.58 mmol, 1.1 eq), Pd2(dba)3 (821 mg, 0.90 mmol, 0.15 eq), BINAP (1.12 g, 1.80 mmol, 0.3 eq) and NaOtBu (1.44 mg, 14.95 mmol, 2.5 eq) and followed by toluene (20 mL) via syringe. The resulting mixture was stirred at 100° C. overnight under a nitrogen atmosphere before it was allowed to cool to room temperature and diluted with water (50 mL) and extracted with EtOAc (2×30 mL). The combined organic layer was washed with water (50 mL) and brine (10 mL) and concentrated. The residue was purified by flash column chromatography on silica gel to give tert-butyl 4-(5-(benzyloxy)pyridin-2-yl)piperazine-1-carboxylate (1.95 g, 92% yield) as a yellow solid.

### Step 3:

To a solution of tert-butyl 4-(5-(benzyloxy)pyridin-2-yl)piperazine-1-carboxylate (500 mg, 1.35 mmol, 1.0 eq) in 1,4-dioxane (2 mL) was added 4N HCl solution in dioxane (1.7 mL, 6.767 mmol, 5.0 eq). The reaction mixture was stirred overnight to produce a precipitate. The solid was collected by filtration and dried to provide 1-(5-(benzyloxy)-pyridin-2-yl)piperazine hydrochloride (380 mg, 92%) as a white solid.

### Step 4:

A mixture of 1-(5-(benzyloxy)pyridin-2-yl)piperazine hydrochloride (380 mg, 1.243 mmol, 1.0 eq) in MeOH (2 mL) and Pd/C catalyst (190 mg, 50% w/w) under hydrogen atmosphere (1 atm) was stirred 5 h before the catalyst was filtered off. The filtrate was concentrated and the residue was purified by reversed phase chromatography (water and ACN as eluent) to give 6-(piperazin-1-yl)pyridin-3-ol hydrochloride (250 mg, 93% yield) as a yellow solid.

### Step 5:

The solution of 2-chloro-6-(1H-indazol-1-yl)-9H-purine (1.0 g, 3.69 mmol, 1 eq) in THF (15 mL) under a N2 atmosphere at 0° C. was added a solution of 1M TBAF in THF (7.39 mL, 7.39 mmol, 2 eq). The reaction mixture was stirred for 15 min followed by addition of ethyl 2-bromoacetate (0.92 g, 5.54 mmol, 1.5 eq). The resulting mixture was stirred from 0-25° C. over a period of 3 h before it was quenched with saturated aq. NH4Cl (50 mL) and extracted with EtOAc (150 mL). The organic layer was washed with brine, dried over Na₂SO₄ and concentrated. The crude residue was purified by flash column chromatography on silica gel (1-50% of EtOAc in petroleum ether) to provide ethyl 2-(2-chloro-6-(1H-indazol-1-yl)-9H-purin-9-yl)acetate (650 mg) and ethyl 2-(2-chloro-6-(1H-indazol-1-yl)-7H-purin-7-yl)acetate (158 mg) as both white solids.

### Step 6:

The solution of ethyl 2-(2-chloro-6-(1H-indazol-1-yl)-9H-purin-9-yl)acetate (199 mg, 0.56 mmol, 0.4 eq) and 6-(piperazin-1-yl)pyridin-3-ol hydrochloride (250 mg, 1.39 mmol, 1.0 eq) in DMSO (5 mL) was stirred at 100° C. overnight under a nitrogen atmosphere. The mixture was allowed to cool to room temperature and diluted with water (20 mL) to produce a precipitate. The solid was collected by filtration and recrystallized in EtOAc to give ethyl 2-(2-(4-(5-hydroxypyridin-2-yl)piperazin-1-yl)-6-(1H-indazol-1-yl)-9H-purin-9-yl)acetate (200 mg, 29% yield) as a brown solid.

### Step 7:

To a solution of ethyl 2-(2-(4-(5-hydroxypyridin-2-yl)piperazin-1-yl)-6-(1H-indazol-1-yl)-9H-purin-9-yl)acetate (100 mg, 0.20 mmol, 1.0 eq) in MeOH (5 mL) and water (5 mL) at 25° C. was added LiOH (42 mg, 1.0 mmol, 5.0 eq). The resulting mixture was stirred at 40° C. for 3 hours before it was concentrated under reduced pressure. The residue was re-dissolved in water (2 mL) and its pH was adjusted to 5 with 2N aq. HCl solution. The precipitate was collected by filtration and purified by Prep-HPLC (Column. Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(5-hydroxypyridin-2-yl)piperazin-1-yl)-6-(1H-indazol-1-yl)-9H-purin-9-yl)acetic acid (16 mg) as an off-white solid.

¹H NMR (400 MHz, CD3OD-d4): δ 9.64 (s, 1H), 8.41 (s, 1H), 7.85-7.71 (m, 3H), 7.51 (d, J=2.8 Hz, 2H), 7.43-7.41 (m, 2H), 7.19-7.13 (m, 1H), 5.11 (s, 2H), 4.25-4.19 (m, 4H), 3.80-3.73 (m, 4H); LC/MS (ESI-MS): [M+H]+=472.1.

### Example 22. Synthesis of 2-(2-(4-(5-hydroxypyridin-2-yl)piperazin-1-yl)-6-(1H-indazol-1-yl)-7H-purin-7-yl)acetic acid

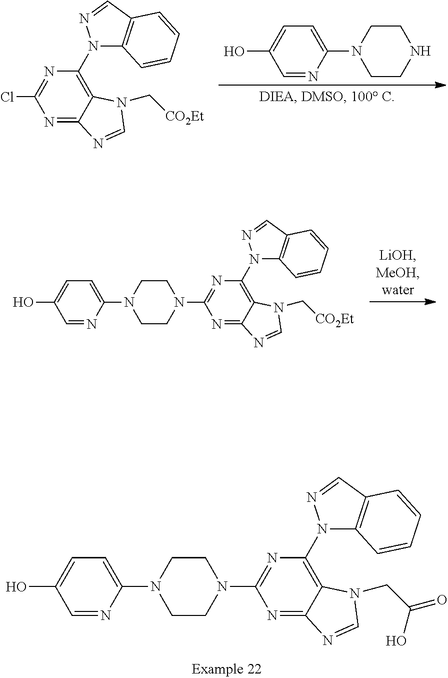 [SMILES: CCOC(=O)Cn1cnc2nc(Cl)nc(-n3ncc4ccccc43)c21 ; Oc1ccc(N2CCNCC2)nc1 ; CCOC(=O)Cn1cnc2nc(N3CCN(c4ccc(O)cn4)CC3)nc(-n3ncc4ccccc43)c21 ; O=C(O)Cn1cnc2nc(N3CCN(c4ccc(O)cn4)CC3)nc(-n3ncc4ccccc43)c21]

The proceeding example was prepared according to Example 21 substituting ethyl 2-(2-chloro-6-(1H-indazol-1-yl)-9H-purin-9-yl)acetate with ethyl 2-(2-chloro-6-(1H-indazol-1-yl)-7H-purin-7-yl)acetate. The desired product 2-(2-(4-(5-hydroxypyridin-2-yl)piperazin-1-yl)-6-(1H-indazol-1-yl)-7H-purin-7-yl)acetic acid was isolated as a white solid. ¹H NMR (400 MHz, CD3OD-d4): δ 8.81 (s, 1H), 8.76-8.74 (m, 2H), 8.57 (s, 1H), 7.97 (d, J=8 Hz, 2H), 7.71-7.64 (m, 2H), 7.56 (s, 1H), 7.55-7.47 (m, 1H), 7.37 (d, J=9.6 Hz, 1H), 5.16 (s, 2H), 4.27-4.21 (m, 4H), 3.88-3.78 (m, 4H); LC/MS(ESI-MS): [M+H]+=472.2.

### Example 23. 4-(4-(9-(3-hydroxypropyl)-6-(1H-indazol-1-yl)-9H-purin-2-yl)piperazin-1-yl)phenol

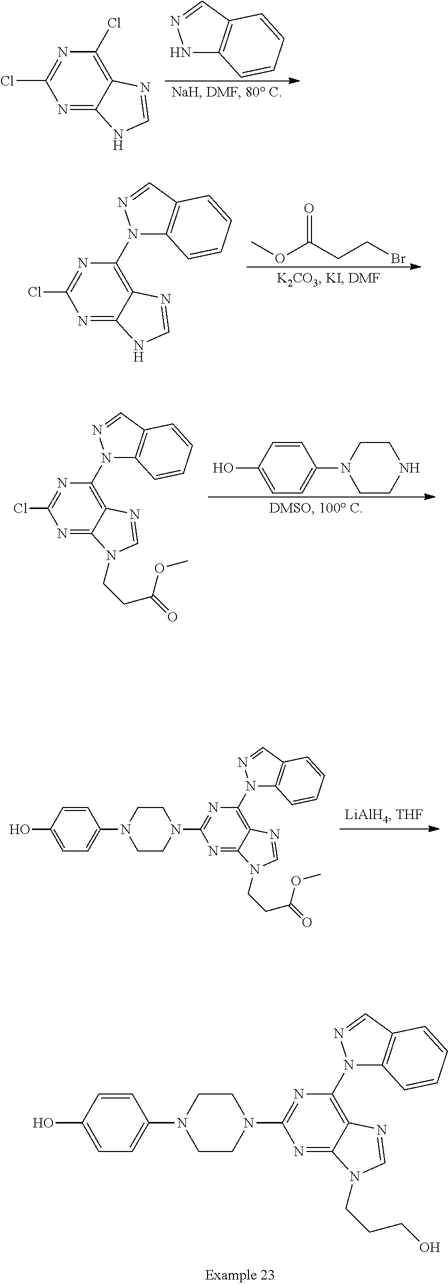

### Step 1:

To a solution of indazole (625 mg, 5.29 mmol, 1.0 eq) in DMF (10 mL) at 25° C. was added NaH (423 mg, 10.58 mmol, 2.0 eq). The mixture was stirred at room temperature for 30 min and followed by addition of a solution of 2,6-dichloro-9H-purine (1 g, 5.29 mmol, 1.0 eq) in DMF (4 mL) dropwise. The resulting mixture was then heated at 80° C. and stirred for overnight. The reaction was allowed to cool and diluted with water (5 mL) and MeCN (10 mL). The resulting precipitate was collected by filtration and dried to 2-chloro-6-(1H-indazol-1-yl)-9H-purine (900 mg, 70% yield) as a white solid.

### Step 2:

To a solution of -chloro-6-(1H-indazol-1-yl)-9H-purine (500 mg, 1.85 mmol, 1.0 eq), K₂CO₃ (919 mg, 6.65 mmol, 3.6 eq) and KI (111.2 mg, 0.67 mmol, 0.36 eq) in DMF (10 mL) at 25° C. under a nitrogen atmosphere was added methyl 3-bromopropanoate (925 mg, 5.54 mmol, 3.0 eq). The mixture was stirred overnight at 50° C. The reaction was allowed to cool and diluted with water (30 mL) and the resulting precipitate was collected by filtration. The filtrate was extracted with EtOAc (50 mL) and the organic layer was concentrated. The combined crude residue was purified by flash column chromatography (DCM:MeOH=10:1) to give methyl 3-(2-chloro-6-(1H-indazol-1-yl)-9H-purin-9-yl)propanoate (70 mg, 11% yield).

### Step 3:

A mixture of methyl 3-(2-chloro-6-(1H-indazol-1-yl)-9H-purin-9-yl)propanoate (70 mg, 0.196 mmol, 1.0 eq) and 4-(piperazin-1-yl)phenol (174.3 mg, 0.98 mmol, 5.0 eq) in DMSO (1 mL) under a nitrogen atmosphere was stirred overnight at 100° C. The reaction was allowed to cooled and partitioned between EtOAc (20 mL) and water (20 mL). The organic layer was washed with water, brine, dried (MgSO4) and concentrated. The crude residue was purified with Prep-HPLC (Column: Waters SunFire Prep C18 OBD 5 um 19*150 mm Column; Mobile phase A: Acetonitile; Mobile phase B: 0.1% TFA in Water; Flow rate: 20.0 ml/min; Detection UV: 210 nm) to give methyl 3-(2-(4-(4-hydroxyphenyl)piperazin-1-yl)-6-(1H-indazol-1-yl)-9H-purin-9-yl)propanoate (40 mg, 41% yield) as a white solid.

### Step 4:

To a solution of methyl 3-(2-(4-(4-hydroxyphenyl)piperazin-1-yl)-6-(1H-indazol-1-yl)-9H-purin-9-yl)propanoate (40 mg, 0.08 mmol, 1.0 eq) in THF (3 mL) under nitrogen atmosphere at 0° C. was added a solution of LiAlH4 in THF (0.16 mL, 0.40 mmol, 5.0 eq, 2.5 mmol/L) dropwise. Then the mixture was stirred for 3 hours at room temperature before it was quenched with water (2 mL) and then diluted with 1N NaOH aq. Solution (1 mL) and THF (2 mL). The solid was removed by filtration and the filtrate was concentrated to provide the crude product. The crude residue was further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(9-(3-hydroxypropyl)-6-(1H-indazol-1-yl)-9H-purin-2-yl)piperazin-1-yl)phenol (6.9 mg, 18% yield) as an off-white solid.

¹H NMR (400 MHz, DMSO-d6): δ 8.86 (br, 1H), 8.61-8.57 (m, 2H), 8.17 (s, 1H), 7.94 (d, J=8.0 Hz, 1H), 7.62 (t, J=7.6 Hz, 1H), 7.39 (t, J=7.6 Hz, 1H), 7.89 (d, J=7.6 Hz, 2H), 7.69 (d, J=7.6 Hz, 2H), 4.66 (s, 1H), 4.24-4.21 (m, 2H), 4.08-3.93 (m, 4H), 3.45 (t, J=5.6 Hz, 1H), 3.19-3.08 (m, 4H), 2.03-1.98 (m, 2H); LC/MS(ESI-MS): [M+H]+=471.2.

### Example 24. 2-(6-(1H-benzo[d][1,2,3]triazol-1-yl)-2-(4-(4-hydroxyphenyl)piperazin-1-yl)-9H-purin-9-yl)acetic acid

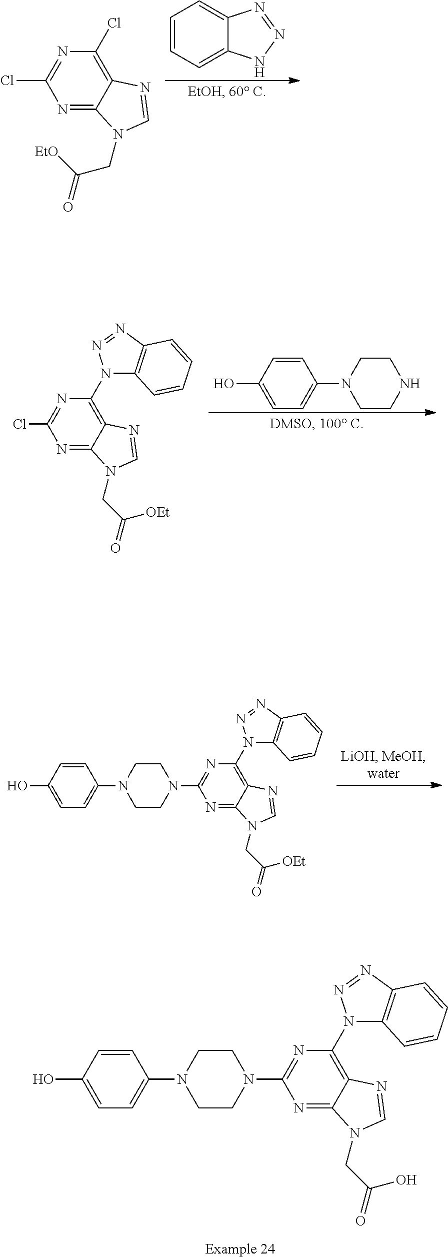 [SMILES: CCOC(=O)Cn1cnc2c(Cl)nc(Cl)nc21 ; c1ccc2[nH]nnc2c1 ; CCOC(=O)Cn1cnc2c(-n3nnc4ccccc43)nc(Cl)nc21 ; Oc1ccc(N2CCNCC2)cc1 ; CCOC(=O)Cn1cnc2c(-n3nnc4ccccc43)nc(N3CCN(c4ccc(O)cc4)CC3)nc21 ; O=C(O)Cn1cnc2c(-n3nnc4ccccc43)nc(N3CCN(c4ccc(O)cc4)CC3)nc21]

### Step 1:

A mixture of ethyl 2-(2,6-dichloro-9H-purin-9-yl)acetate (2 g, 7.27 mmol, 1.0 eq), 1H-benzo[d][1,2,3]triazole (866 mg, 7.27 mmol, 1.0 eq) in EtOH (30 mL) and DIEA (2.8 g, 21.81 mmol, 3.0 eq) was stirred under a nitrogen atmosphere at 60° C. for overnight before it was allowed to cool to 25° C. The resulting solid was collect by filtration and washed with EtOH (2×10 mL) and dried to provide ethyl 2-(6-(1H-benzo[d][1,2,3]triazol-1-yl)-2-chloro-9H-purin-9-yl)acetate (2.28 g, 88% yield) as a white solid.

### Step 2:

To a solution of ethyl 2-(6-(1H-benzo[d][1,2,3]triazol-1-yl)-2-chloro-9H-purin-9-yl)acetate (2 g, 7.27 mmol, 1.0 eq) in DMSO (25 mL) was added 4-(piperazin-1-yl)phenol (2.27 g, 12.75 mmol, 2.0 eq). The resulting mixture was stirred at 100° C. for overnight under nitrogen atmosphere before it was cooled to room temperature and diluted with water (50 mL). The resulting solid was collected by filtration and dried to give ethyl 2-(6-(1H-benzo[d][1,2,3]triazol-1-yl)-2-(4-(4-hydroxyphenyl)piperazin-1-yl)-9H-purin-9-yl)acetate (2.8 g, 88% yield) as a brown solid.

### Step 3:

To a solution of ethyl 2-(6-(1H-benzo[d][1,2,3]triazol-1-yl)-2-(4-(4-hydroxy phenyl)-piperazin-1-yl)-9H-purin-9-yl)acetate (2 g, 4.0 mmol, 1.0 eq) in water (10 mL) and MeOH (10 mL) was added LiOH (840 mg, 20 mmol, 5.0 eq). The resulting mixture was stirred at room temperature for 3 before the organic volatile was removed under reduced pressure. The aq. solution was acidified to pH ˜4 with 1N aq. HCl. The resulting solid was collected by filtration. This crude solid product was dissolved in DMSO (7.5 mL) and followed by addition of MeOH (15 mL) dropwise. The resulting precipitate was collected by filtration and rinsed with MeOH (2×3 mL) to give 2-(6-(1H-benzo[d][1,2,3]triazol-1-yl)-2-(4-(4-hydroxyphenyl)piperazin-1-yl)-9H-purin-9-yl)acetic acid (1.26 g, 67% yield) as a white solid.

¹H NMR (400 MHz, DMSO-d6): δ 8.48 (d, J=8.4 Hz, 1H), 8.35 (s, 1H), 8.29 (d, J=8.4 Hz, 1H), 7.79 (t, J=8.4 Hz, 1H), 7.62 (t, J=7.6 Hz, 1H), 7.03 (br, 2H), 6.75-6.73 (m, 2H), 5.06 (s, 2H), 4.15-3.85 (m, 4H), 3.37-3.15 (m, 4H); LC/MS(ESI-MS): [M+H]+=472.2.

### Example 25. 2-(2-(4-(4-hydroxyphenyl)piperazin-1-yl)-6-(1H-indol-3-yl)-9H-purin-9-yl)acetic acid

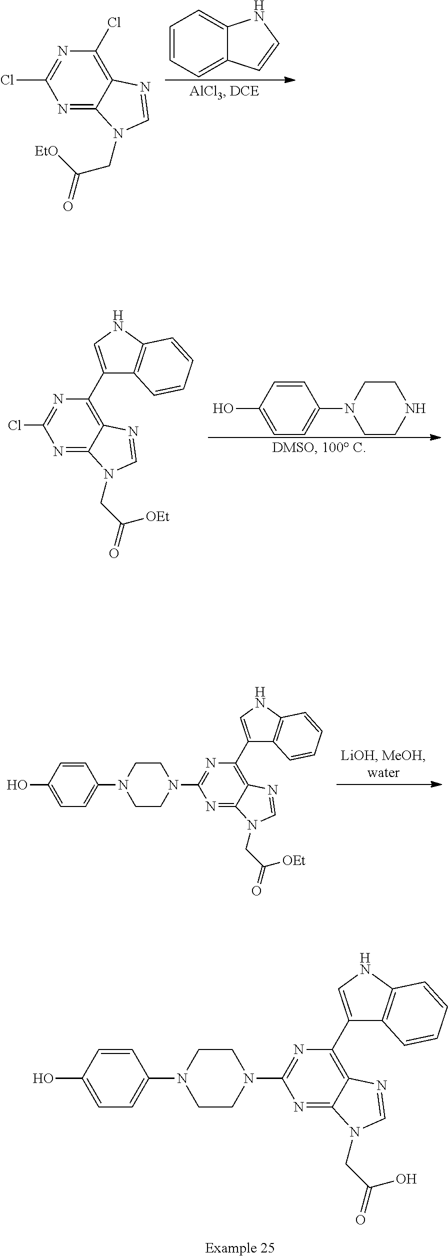

### Step 1:

To a solution of ethyl 2-(2,6-dichloro-9H-purin-9-yl)acetate (400 mg, 1.045 mmol, 1.0 eq) and indole (341 mg, 2.91 mmol, 2.0 eq) in dichloroethane (15 mL) was added AlCl3 (582 mg, 4.36 mmol, 3.0 eq). The resulting mixture was stirred under a nitrogen atmosphere at 80° C. for overnight before it was allowed to cool to room temperature and diluted with water (10 mL). The resulting solid was filtered off and the filtrate was extracted with DCM (3×20 mL). The combined organic layer was concentrated. The residue was purified by flash column chromatography on silica gel (petroleum ether:EtOAc=5:1) to give ethyl 2-(2-chloro-6-(1H-indol-3-yl)-9H-purin-9-yl)acetate (100 mg, 19% yield) as a solid.

### Step 2:

To a solution of ethyl 2-(2,6-dichloro-9H-purin-9-yl)acetate (80 mg, 0.22 mmol, 1.0 eq) in DMSO (2 mL) at room temperature was added 4-(piperazin-1-yl)phenol (120 mg, 0.67 mmol, 3.0 eq). The resulting mixture was stirred at 100° C. for overnight under nitrogen atmosphere before it was cooled to room temperature and diluted with water (5 mL). The resulting solid was collected by filtration and dried to give ethyl 2-(2-(4-(4-hydroxyphenyl)-piperazin-1-yl)-6-(1H-indol-3-yl)-9H-purin-9-yl)acetate (277 mg, 88% yield) as an off-white solid.

### Step 3:

To a solution of ethyl 2-(2-(4-(4-hydroxyphenyl)-piperazin-1-yl)-6-(1H-indol-3-yl)-9H-purin-9-yl)acetate (277 mg, 0.56 mmol, 1.0 eq) in water (2.5 mL) and MeOH (2.5 mL) was added LiOH (117 mg, 2.78 mmol, 5.0 eq). The resulting mixture was stirred at room temperature for 3 hours before the organic volatile was removed under reduced pressure. The aq. solution was then acidified to pH ˜4 with 1N aq. HCL. The resulting precipitate was collected by filtration. The crude solid was then purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(1H-indol-3-yl)-9H-purin-9-yl)acetic acid (15 mg, 4% yield) as solid.

¹H NMR (400 MHz, DMSO-d6): δ 13.29 (br, 1H), 11.91 (s, 1H), 9.36 (br, 1H), 8.99 (s, 1H), 8.63 (d, J=7.6 Hz, 1H), 8.16 (s, 1H), 7.53 (d, J=6.8 Hz, 1H), 7.24-7.03 (m, 4H), 6.80 (d, J=7.6 Hz, 2H), 4.97 (s, 2H), 4.15-3.85 (m, 4H), 3.47-3.23 (m, 4H); LC/MS(ESI-MS): [M+H]+=470.1.

### Example 26. 2-(2-(4-(4-hydroxyphenyl)piperazin-1-yl)-6-(isoindolin-2-yl)-9H-purin-9-yl)acetic acid

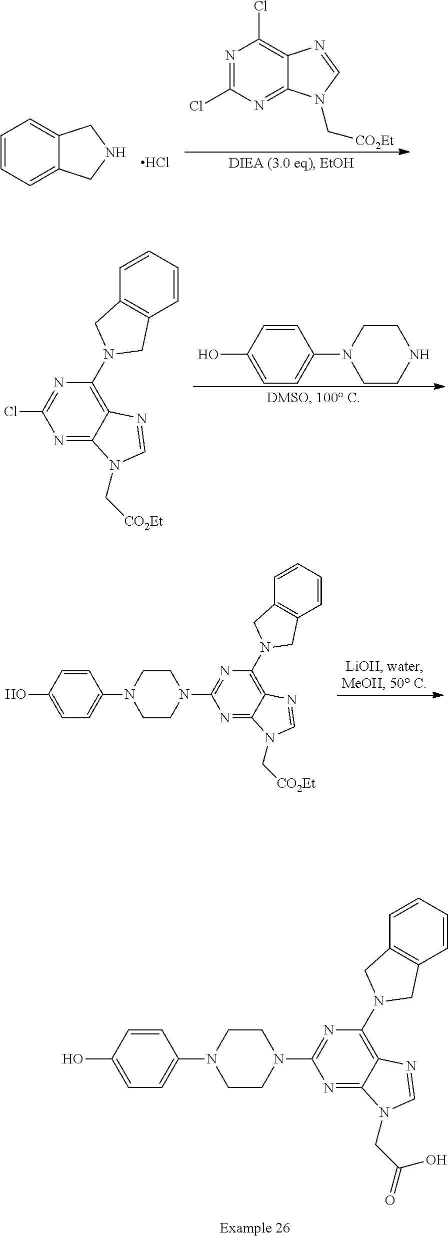

### Step 1:

A mixture of ethyl 2-(2,6-dichloro-9H-purin-9-yl)acetate (300 mg, 1.09 mmol, 1.0 eq), isoindoline hydrochloride (178 mg, 1.15 mmol, 1.05 e) in EtOH (5 mL) was added DIEA (422 mg, 3.27 mmol, 3.0 eq). The reaction was stirred at 45° C. for 1 hour before it was allowed to cool to room temperature. The resulting precipitate was collected by filtration and dried to give ethyl 2-(2-chloro-6-(isoindolin-2-yl)-9H-purin-9-yl)acetate (340 mg, 87% yield) as a white solid.

### Step 2:

To a solution of ethyl 2-(2-chloro-6-(isoindolin-2-yl)-9H-purin-9-yl)acetate (340 mg, 0.95 mmol, 1.0 eq) in DMSO (5 mL) was added 4-(piperazin-1-yl)phenol (847 mg, 4.75 mmol, 5.0 eq). The resulting mixture was stirred at 100° C. for overnight under nitrogen atmosphere before it was cooled to room temperature and diluted with water (5 mL). The resulting solid was collected by filtration and dried to give ethyl 2-(2-(4-(4-hydroxyphenyl)piperazin-1-yl)-6-(isoindolin-2-yl)-9H-purin-9-yl)acetate (475 mg, 98% yield) as an off-white solid.

### Step 3:

To a solution of ethyl 2-(2-(4-(4-hydroxyphenyl)piperazin-1-yl)-6-(isoindolin-2-yl)-9H-purin-9-yl)acetate (475 mg, 0.95 mmol, 1.0 eq) in water (2.5 mL) and MeOH (2.5 mL) was added LiOH (120 mg, 2.85 mmol, 5.0 eq). The resulting mixture was stirred at room temperature for 3 hours before the organic volatile was removed under reduced pressure. The aq. solution was then acidified to pH ˜4 with 1N aq. HCl. The resulting precipitate was collected by filtration. The crude solid was then purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(isoindolin-2-yl)-9H-purin-9-yl)acetic acid (32 mg, 7% yield) as an off-white solid.

¹H NMR (400 MHz, MeOD-d4): δ 8.06 (s, 1H), 7.5-7.49 (m, 2H), 7.46-7.39 (m, 2H), 7.38-7.33 (m, 2H), 6.97 (d, J=8.8 Hz, 1H), 5.40-5.31 (m, 2H), 5.11-4.97 (m, 2H), 4.96 (s, 2H), 4.41-4.16 (m, 4H), 3.73-3.65 (m, 4H); LC/MS(ESI-MS): [M+H]+=472.2.

### Example 27. 2-(2,6-bis(1-oxoisoindolin-2-yl)-9H-purin-9-yl)acetic acid

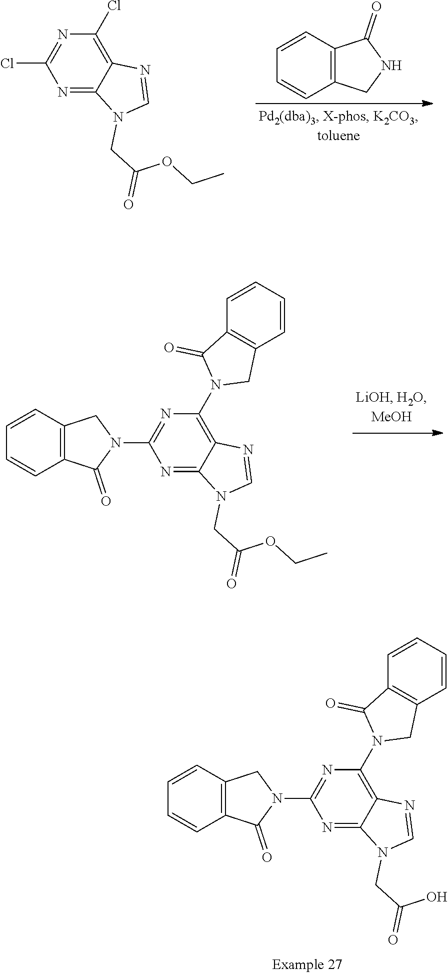 [SMILES: CCOC(=O)Cn1cnc2c(Cl)nc(Cl)nc21 ; O=C1NCc2ccccc21 ; CCOC(=O)Cn1cnc2c(N3Cc4ccccc4C3=O)nc(N3Cc4ccccc4C3=O)nc21 ; O=C(O)Cn1cnc2c(N3Cc4ccccc4C3=O)nc(N3Cc4ccccc4C3=O)nc21]

### Step 1:

To a solution of ethyl 2-(2,6-dichloro-9H-purin-9-yl)acetate (200 mg, 0.58 mmol, 1.0 eq) in toluene (5 mL) under a nitrogen atmosphere was added isoindolin-1-one (77 mg, 0.58 mmol, 1.0 eq), Pd2(dba)3 (80 mg, 0.09 mmol, 0.15 eq), X-phos (83 mg, 0.17 mmol, 0.3 eq) and K2CO3 (201 mg, 1.45 mmol, 2.5 eq). The mixture was stirred at 80° C. overnight. The reaction mixture was allowed to cool and diluted with water (30 mL) and extracted with EtOAc (3×50 mL). The combined organic layer was washed with brine, dried over Na₂SO₄ and concentrated. The residue was purified by flash column chromatography on silica gel (petroleum ether:EtOAc=1:1) to give ethyl 2-(2,6-bis(1-oxoisoindolin-2-yl)-9H-purin-9-yl)acetate (0.13 g, 48% yield).

### Step 2:

To a solution of ethyl 2-(2,6-bis(1-oxoisoindolin-2-yl)-9H-purin-9-yl)acetate (32 mg, 0.07 mmol, 1.0 eq) in MeOH (3 mL) and water (3 mL) was added LiOH.H₂O (9 mg, 0.21 mmol 3.0 eq). The reaction was stirred for 0.5 h at room temperature before it was concentrated. The residue was diluted with water (2 mL) and adjusted its pH to 3 with 2N aq. HCl. The resulting solid was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2,6-bis(1-oxoisoindolin-2-yl)-9H-purin-9n-9-acetic acid (5.4 mg, 18% yield) as a white solid.

¹H NMR (400 MHz, MeOD-d4): 8.67 (s, 1H), 8.03-7.88 (m, 2H), 7.83-7.72 (m, 4H), 7.66-7.52 (m, 2H), 5.50 (s, 2H), 5.33 (s, 2H), 5.26 (s, 2H); LC/MS(ESI-MS): [M+H]+=441.2.

### Example 28. 2-(2-(4-(4-hydroxyphenyl)piperazin-1-yl)-6-(2-methyl-1H-benzo[d]imidazol-1-yl)-9H-purin-9-yl)acetic acid

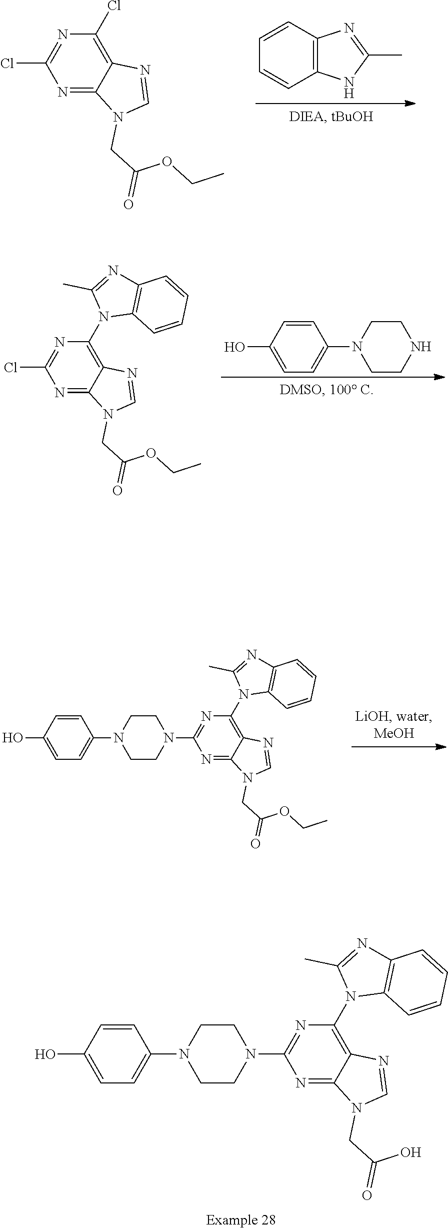

The proceeding example was prepared according to Example 24 substituting 1H-benzo[d][1,2,3]triazole with 2-methyl-1H-benzo[d]imidazole in the presence of DIEA. The desired product 2-(2-(4-(4-hydroxyphenyl)piperazin-1-yl)-6-(2-methyl-1H-benzo[d]imidazol-1-yl)-9H-purin-9-yl)acetic acid was isolated as a white solid.

¹H NMR (300 MHz, DMSO-d6): δ 8.35 (s, 1H), 7.73 (d, J=6.9 Hz, 1H), 7.65 (d, J=7.5 Hz, 1H), 7.41-7.23 (m, 4H), 6.80 (d, J=8.7 Hz, 1H), 5.04 (s, 2H), 4.21-4.05 (m, 4H), 3.44-3.35 (m, 4H), 2.81 (s, 3H); LC/MS(ESI-MS): [M+H]+=485.2.

### Example 29. 2-(6-(2-(hydroxymethyl)-1H-benzo[d]imidazol-1-yl)-2-(4-(4-hydroxyphenyl)piperazin-1-yl)-9H-purin-9-yl)acetic acid

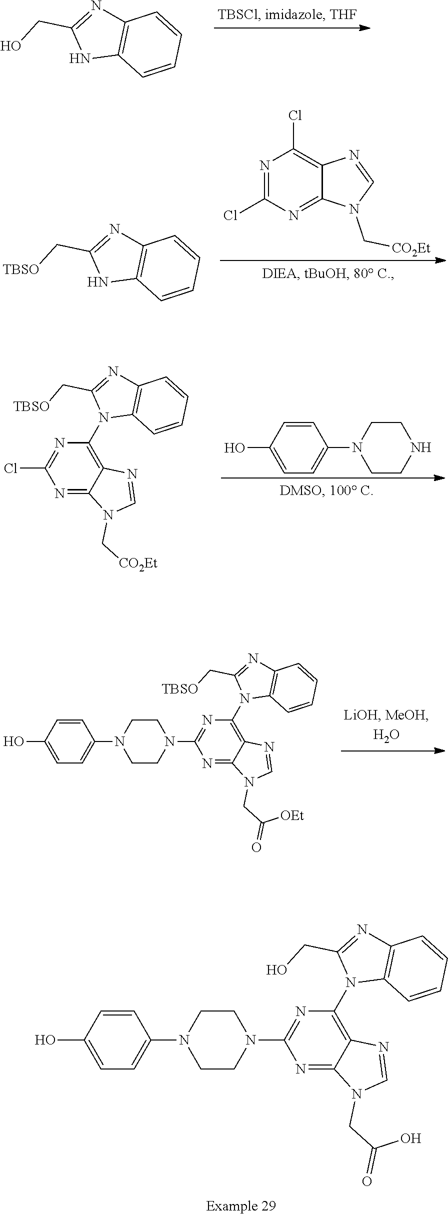

### Step 1:

To a solution of (1H-benzo[d]imidazol-2-yl)methanol (500 mg, 3.37 mmol, 1.0 eq) in THF (5 mL) at 0° C. was added imidazole (459 mg, 6.75 mmol, 2.0 eq) and TBSCl (763 mg, 5.06 mmol, 1.5 eq). The mixture was stirred at room temperature for 3 hours before it was quenched with water (15 mL) and extracted with DCM (2×30 mL). The combined organic layer was dried over Na₂SO₄ and concentrated to give crude 2-(((tert-butyldimethylsilyl)-oxy)methyl)-1H-benzo[d]imidazole (940 mg).

### Step 2:

A mixture of crude 2-(((tert-butyldimethylsilyl)-oxy)methyl)-1H-benzo[d]imidazole (940 mg, 3.58 mmol, 1.1 eq), ethyl 2-(2,6-dichloro-9H-purin-9-yl)acetate (94 mg, 3.26 mmol, 1.0 eq) and DIEA (1.05 g, 8.14 mmol, 2.5 eq) in t-BuOH (10 mL) was stirred at 70° C. for overnight. The reaction was allowed to cool and diluted with water (50 mL) and extracted with EtOAc (2×50 mL). The combined organic layer was dried over Na₂SO₄ and concentrated. The residue was purified by flash column chromatography on silica gel (petroleum ether:EtOAc=5:1) to give ethyl 2-(6-(2-(((tert-butyldimethylsilyl)oxy)methyl)-1H-benzo[d]imidazol-1-yl)-2-chloro-9H-purin-9-yl)acetate (150 mg, 9% yield over 2 steps) as a white solid.

### Step 3:

A mixture of ethyl 2-(6-(2-(((tert-butyldimethylsilyl)oxy)methyl)-1H-benzo[d]imidazol-1-yl)-2-chloro-9H-purin-9-yl)acetate (150 mg, 0.30 mmol, 1.0 eq) and 4-(piperazin-1-yl)-phenol (267 mg, 1.50 mmol, 5.0 eq) in DMSO (3 mL) was stirred at 100° C. for overnight. The reaction was allowed to cool and diluted with water (15 mL). The resulting solid was collected by filtration and dried to give crude ethyl 2-(6-(2-(((tert-butyldimethylsilyl)oxy)-methyl)-1H-benzo[d]imidazol-1-yl)-2-(4-(4-hydroxyphenyl)piper-azin-1-yl)-9H-purin-9-yl)acetate (370 mg) which was used in next step without further purification.

### Step 4:

A mixture of crude ethyl 2-(6-(2-(((tert-butyldimethylsilyl)oxy)methyl)-1H-benzo[d]-imidazol-1-yl)-2-(4-(4-hydroxyphenyl)piper-azin-1-yl)-9H-purin-9-yl)acetate (370 mg, 0.57 mmol, 1 eq) and LiOH.H2O (120.5 mg, 2.87 mmol, 5 eq) in MeOH (3 mL) and water (3 mL) was stirred at 50° C. for 4 hours. The reaction was allowed to cool to room temperature and concentrated under reduced pressure. The residue was diluted with water (5 mL) and its pH was adjusted to 4 with 2N HCl aqueous solution. The resulting precipitate was collected and dried. The solid was further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(6-(2-(hydroxymethyl)-1H-benzo[d]imidazol-1-yl)-2-(4-(4-hydroxyphenyl)piperazin-1-yl)-9H-purin-9-yl)acetic acid (7.9 mg, 2% yield) as a white solid.

¹H NMR (400 MHz, MeOD-d4): δ 8.13 (s, 1H), 7.89-7.75 (m, 2H), 7.62-7.54 (m, 2H), 7.39-7.35 (m, 2H), 6.90 (d, J=8.4 Hz, 2H), 6.07 (s, 2H), 5.04-4.99 (m, 4H), 4.21-4.11 (m, 4H), 3.58-3.51 (m, 4H), 5.40-5.31 (m, 2H), 5.11-4.97 (m, 2H), 4.96 (s, 2H), 4.41-4.16 (m, 4H), 3.73-3.65 (m, 4H); LC/MS(ESI-MS): [M+H]+=501.2.

### Example 30. 4-(4-(9-(1-hydroxybutan-2-yl)-6-(indolin-1-yl)-9H-purin-2-yl)piperazin-1-yl)phenol

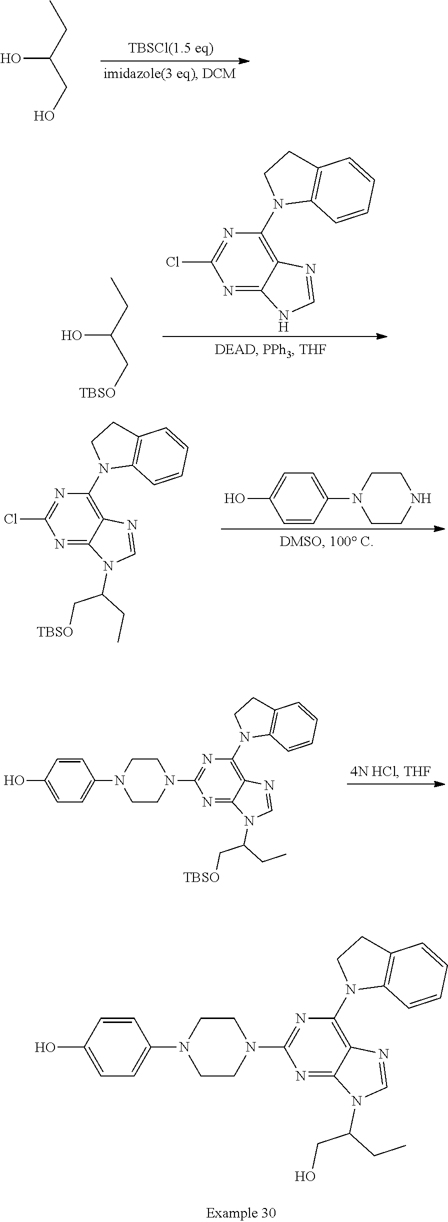

### Step 1:

To a solution of 1,2-butanediol (3 g, 33.3 mmol, 1.0 eq) in DCM (30 mL) at 0° C. was added TBSCl (7.5 g, 50.0 mmol, 1.5 eq) and imidazol (6.8 g, 100.0 mmol, 3 eq). The mixture was stirred at 25° C. overnight before it was concentrated under reduced pressure. The residue was diluted with water (30 mL) and extracted with EtOAc (3×30 mL). The combined organic layer was washed with brine, dried over Na₂SO₄, concentrated. The residue was purified by flash column chromatography on silica gel (petroleum ether:EtOAc=5:1) to give 1-((tert-butyldimethylsilyl)oxy)butan-2-ol (1.2 g, 18% yield).

### Step 2:

To a solution of 1-((tert-butyldimethylsilyl)oxy)butan-2-ol (0.31 g, 1.18 mmol, 1.5 eq), 2-chloro-6-(indolin-1-yl)-9H-purine (0.2 g, 0.79 mmol, 1.0 eq) and PPh3 (0.24 g, 0.79 mmol, 1.5 eq) in THF (10 mL) under a nitrogen atmosphere at 0° C. was added DBAD (0.24 g, 0.79 mmol, 1.5 eq). The reaction mixture was stirred from 0-25° C. overnight before it was concentrated under reduced pressure. The residue was partitioned between water (10 mL) and EtOAc (20 mL). The organic layer was washed with brine, dried over Na₂SO₄, concentrated. The residue was purified by flash column chromatography on silica gel to give 9-(1-((tert-butyldimethylsilyl)oxy)butan-2-yl)-2-chloro-6-(indolin-1-yl)-9H-purine (0.3 g, 86% yield).

### Step 3:

A mixture of 9-(1-((tert-butyldimethylsilyl)oxy)butan-2-yl)-2-chloro-6-(indolin-1-yl)-9H-purine (0.3 g, 0.66 mmol, 1.0 eq) and 1-(4-hydroxyphenyl) piperazine (0.58 g, 3.28 mmol, 5.0 eq) in DMSO (8 mL) under a nitrogen atmosphere was stirred overnight at 100° C. The reaction was allowed to cool and diluted with water (30 mL). The resulting solid was collected by filtration and dried to give 4-(4-(9-(1-((tert-butyldimethylsilyl)oxy)butan-2-yl)-6-(indolin-1-yl)-9H-purin-2-yl)piperazin-1-yl)phenol (0.35 g, 87% yield) as a solid.

### Step 4:

To a solution of 4-(4-(9-(1-((tert-butyldimethylsilyl)oxy)butan-2-yl)-6-(indolin-1-yl)-9H-purin-2-yl)piperazin-1-yl)phenol (0.35 g, 0.65 mmol, 1.0 eq) in THF (8 mL) at 25° C. was added a solution of 4N aq. HCl (6.5 mL) was added. The mixture was stirred for 0.5 h before it was concentrated. The residue was re-dissolved in EtOAc and washed with saturated aq. NaHCO₃, brine, dried over Na₂SO₄, and concentrated. The residue was purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(9-(1-hydroxybutan-2-yl)-6-(indolin-1-yl)-9H-purin-2-yl)piperazin-1-yl)phenol (217 mg, 72% yield) as a white solid.

¹H NMR (400 MHz, DMSO-d6): δ 8.30 (d, J=8.0 Hz, 1H), 8.19 (s, 1H), 7.75-7.64 (m, 2H), 7.30 (d, J=7.2 Hz, 1H), 7.22 (t, J=8.0 Hz, 1H), 6.98 (t, J=7.2 Hz, 1H), 6.90 (d, J=8.8 Hz, 1H), 4.73 (t, J=8.4 Hz, 1H), 4.44-4.27 (m, 1H), 3.94-3.83 (m, 1H), 3.77-3.55 (m, 6H), 3.25 (t, J=8.4 Hz, 2H), 2.54 (s, 1H), 1.99-1.93 (m, 2H), 0.80 (t, J=7.4 Hz, 1H); LC/MS(ESI-MS): [M+H]+=486.2.

### Example 31. 2-(2-(4-(4-hydroxyphenyl)piperazin-1-yl)-6-(indolin-1-yl)-9H-purin-9-yl)acetic acid

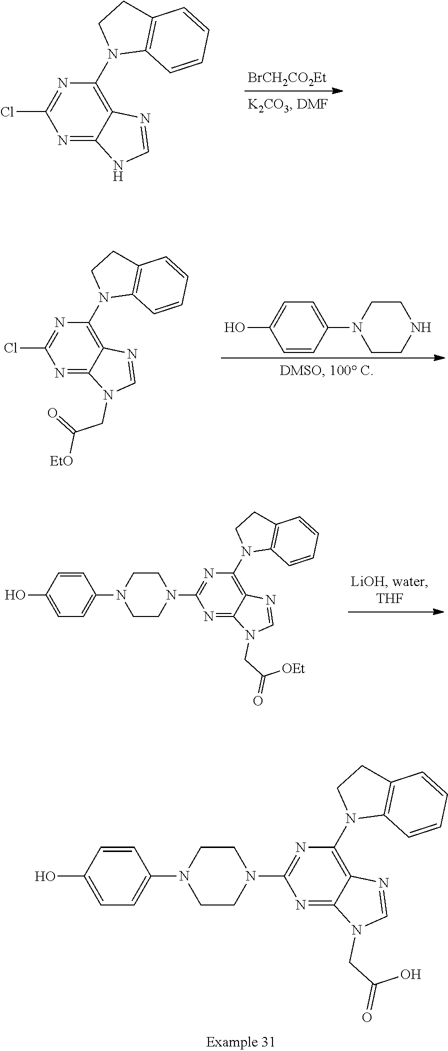 [SMILES: Clc1nc(N2CCc3ccccc32)c2nc[nH]c2n1 ; CCOC(=O)Cn1cnc2c(N3CCc4ccccc43)nc(Cl)nc21 ; Oc1ccc(N2CCNCC2)cc1 ; "BrCH2CO2Et" ; CCOC(=O)Cn1cnc2c(N3CCc4ccccc43)nc(N3CCN(c4ccc(O)cc4)CC3)nc21 ; O=C(O)Cn1cnc2c(N3CCc4ccccc43)nc(N3CCN(c4ccc(O)cc4)CC3)nc21]

### Step 1:

To a solution of 2-chloro-6-(indolin-1-yl)-9H-purine (0.5 g, 1.84 mmol, 1.0 eq) and ethyl bromoaetate (1.23 g, 7.35 mmol, 4.0 eq) at 25° C. in DMF (10 mL) was added K₂CO₃ (1.77 g, 9.07 mmol, 5.0 eq). The mixture was stirred overnight before it was diluted with water (20 mL). The resulting solid was collected by filtration and dried to give crude ethyl 2-(2-chloro-6-(indolin-1-yl)-9H-purin-9-yl)acetate (0.3 g, 45%) as a white solid.

### Step 2:

To a solution of ethyl 2-(2-chloro-6-(indolin-1-yl)-9H-purin-9-yl)acetate (0.3 g, 1.10 mmol, 1.0 eq) in DMSO (5 mL) 1-(4-Hydroxyphenyl) piperazine (0.98 g, 0.56 mmol, 3.0 eq). The mixture was stirred at 100° C. for overnight before it was allowed to cool and diluted with water (15 mL). The resulting solid was collected by filtration and dried to give crude methyl 2-(2-(4-(4-hydroxyphenyl)piperazin-1-yl)-6-(indolin-1-yl)-9H-purin-9-yl)acetate (350 mg, 85% yield) as a solid.

### Step 3:

To a solution of crude methyl 2-(2-(4-(4-hydroxyphenyl)piperazin-1-yl)-6-(indolin-1-yl)-9H-purin-9-yl)acetate (300 mg, 0.60 mmol, 1.0 eq) in THF (3 mL) and water (3 mL) was added LiOH (72 mg, 3.18 mmol, 5 eq). The resulting mixture was stirred at room temperature for 3 hours before the organic volatile was removed under reduced pressure. The aq. solution was acidified to pH 4 with 2N aq. HCl and the precipitate was collected by filtration. The crude product was purified by Prep-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 250 mg of 2-(2-(4-(4-hydroxy-phenyl)piperazin-1-yl)-6-(indolin-1-yl)-9H-purin-9-yl)acetic acid (154 mg) as a white solid.

¹H NMR (400 MHz, DMSO-d6): δ 8.32 (d, J=8.0 Hz, 1H), 8.00 (s, 1H), 7.70-7.41 (m, 2H), 7.29 (d, J=7.2 Hz, 1H), 7.22 (t, J=8.0 Hz, 1H), 6.98 (t, J=7.6 Hz, 1H), 6.87-6.85 (m, 2H), 4.93 (s, 3H), 4.76-4.71 (m, 3H), 4.95-4.91 (m, 1H), 3.62-3.46 (m, 4H), 3.26-3.22 (m, 3H); LC/MS(ESI-MS): [M+H]+=472.1.

### Example 32. 2-(2-(4-(4-hydroxyphenyl)piperazin-1-yl)-6-(indolin-1-yl)-9H-purin-9-yl)acetamide

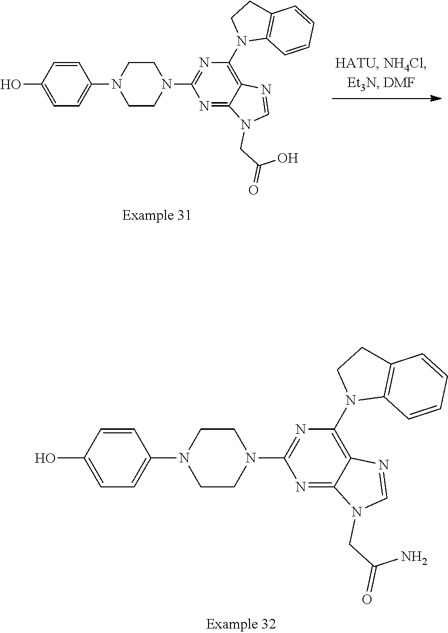 [SMILES: O=C(O)Cn1cnc2c(N3CCc4ccccc43)nc(N3CCN(c4ccc(O)cc4)CC3)nc21 ; NC(=O)Cn1cnc2c(N3CCc4ccccc43)nc(N3CCN(c4ccc(O)cc4)CC3)nc21]

To a solution of 2-(2-(4-(4-hydroxy-phenyl)piperazin-1-yl)-6-(indolin-1-yl)-9H-purin-9-yl)acetic acid (0.16 g, 0.34 mmol, 1.0 eq) in DMF (6 mL) was added HATU (0.14 g, 0.4 mmol, 1.2 eq). The mixture was stirred at 25° C. for 1.5 h and followed by addition of Et3N (65 mg, 0.65 mmol, 2 eq) and NH₄Cl (55 mg, 1.0 mmol, 3 eq). The mixture was stirred at 25° C. overnight before it was diluted with water (20 mL). The resulting precipitate was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(indolin-1-yl)-9H-purin-9-yl)acetamide (41 mg, 26% yield) as a white solid.

¹H NMR (400 MHz, DMSO-d6): δ 8.40 (d, J=8.0 Hz, 1H), 8.01 (s, 1H), 7.78 (s, 1H), 7.38-7.26 (m, 4H), 7.04 (t, J=7.6 Hz, 1H), 6.88 (d, J=8.8 Hz, 1H), 4.81-4.77 (m, 4H), 4.01-3.91 (m, 4H), 3.55-3.41 (m, 4H), 3.33-3.28 (m, 2H); LC/MS(ESI-MS): [M+H]+=458.1.

### Example 33. 2-(6-(3-(ethoxycarbonyl)-1H-indazol-1-yl)-2-(4-(4-hydroxyphenyl)piperazin-1-yl)-9H-purin-9-yl)acetic acid

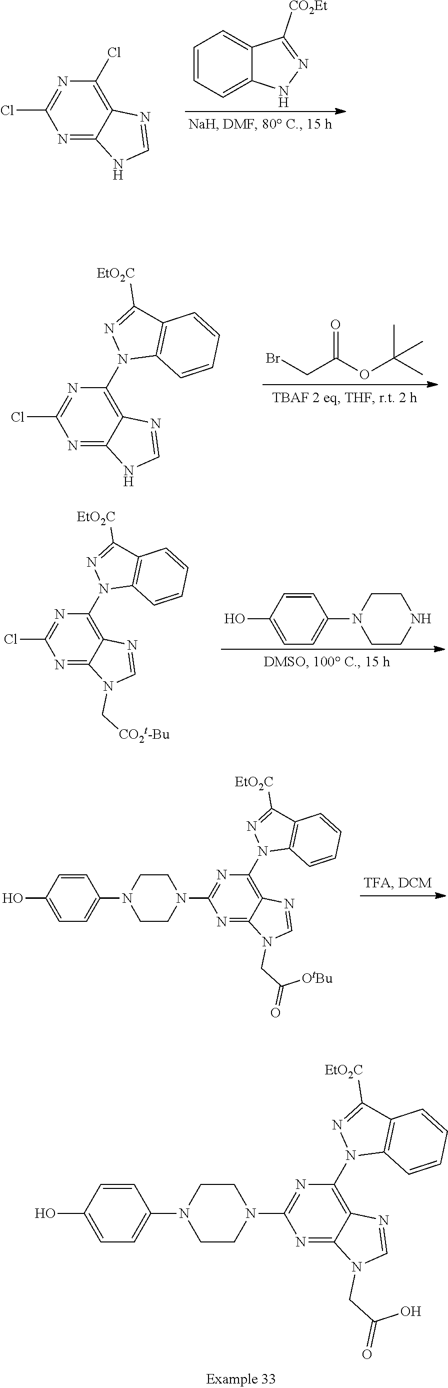

### Step 1:

To a solution of ethyl 1H-indazole-3-carboxylate (0.5 g, 1.71 mmol, 1 eq) in DMF (5 mL) under a nitrogen atmosphere at 0° C. was added sodium hydride (0.21 g, 5.26 mmol, 2 eq) in portions. The mixture was stirred for 0.5 h and followed by addition of 2,6-dichloro-9H-purine (0.49 g, 2.63 mmol, 1 eq) in DMF (2 mL). The resulting mixture was heated to 80° C. overnight before it was allowed to cool and diluted with water (20 mL). The resulting solid was collected by filtration and dried to give ethyl 1-(2-chloro-9H-purin-6-yl)-1H-indazole-3-carboxylate (0.6 g, 45% yield) as a yellow solid.

### Step 2:

To a solution of ethyl 1-(2-chloro-9H-purin-6-yl)-1H-indazole-3-carboxylate (0.54 g, 1.58 mmol, 1.0 eq) in THF (10 mL) at 25° C. was added a solution of TBAF in THF (3.2 mL, 3.16 mmol, 2 eq). The reaction mixture was stirred for 30 min and followed by addition of a solution of tert-butyl bromoacetate (0.61 g, 3.16 mmol, 2 eq) in THF (2 mL) dropwise. The mixture was stirred overnight before it was diluted with water (30 mL) and extracted with EtOAc (3×30 mL). The combined organic layer was washed with brine, dried over Na₂SO₄ and concentrated. The residue was purified by flash column chromatography on silica gel (petroleum ether:EtOAc=1:1) to give ethyl 1-(9-(2-(tert-butoxy)-2-oxoethyl)-2-chloro-9H-purin-6-yl)-1H-indazole-3-carboxylate (0.2 g, 28% yield) as a white solid.

### Step 3:

A mixture of ethyl 1-(9-(2-(tert-butoxy)-2-oxoethyl)-2-chloro-9H-purin-6-yl)-1H-indazole-3-carboxylate (60 mg, 0.13 mmol, 1 eq) and 1-(4-hydroxyphenyl) piperazine (71 mg, 0.4 mmol, 3 eq) in DMSO (3 mL) was stirred at 100° C. overnight. The reaction was allowed to cool and diluted with water (10 mL). The resulting precipitate was collected by filtration and dried to give ethyl 1-(9-(2-(tert-butoxy)-2-oxoethyl)-2-(4-(4-hydroxyphenyl)piperazin-1-yl)-9H-purin-6-yl)-1H-indazole-3-carboxylate (0.12 g, 90% yield) as a solid.

### Step 4:

A mixture of ethyl 1-(9-(2-(tert-butoxy)-2-oxoethyl)-2-(4-(4-hydroxyphenyl)piperazin-1-yl)-9H-purin-6-yl)-1H-indazole-3-carboxylate (0.12 g, 0.20 mmol) in TFA (3 mL) and DCM (3 mL) was stirred for 0.5 h at room temperature before it was concentrated. The residue was purified by Prep-HPLC(Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(6-(3-(ethoxycarbonyl)-1H-indazol-1-yl)-2-(4-(4-hydroxyphenyl)piperazin-1-yl)-9H-purin-9-yl)acetic acid (6 mg, 6% yield) as a white solid.

¹H NMR (400 MHz, MeOD-d4): δ 8.53-8.48 (m, 2H), 8.31-8.29 (m, 1H), 7.69-7.65 (m, 1H), 7.55-7.47 (m, 3H), 6.95 (d, J=9.2 Hz, 2H), 5.66 (s, 2H), 4.58-4.52 (m, 2H), 4.41-4.23 (m, 4H), 3.79-3.71 (m, 4H), 1.53 (t, J=7.2 Hz, 2H); LC/MS(ESI-MS): [M+H]+=543.2.

### Example 34. 2-(2-(4-(4-hydroxyphenyl)piperazin-1-yl)-6-(1H-indazol-4-yl)-9H-purin-9-yl)acetic acid

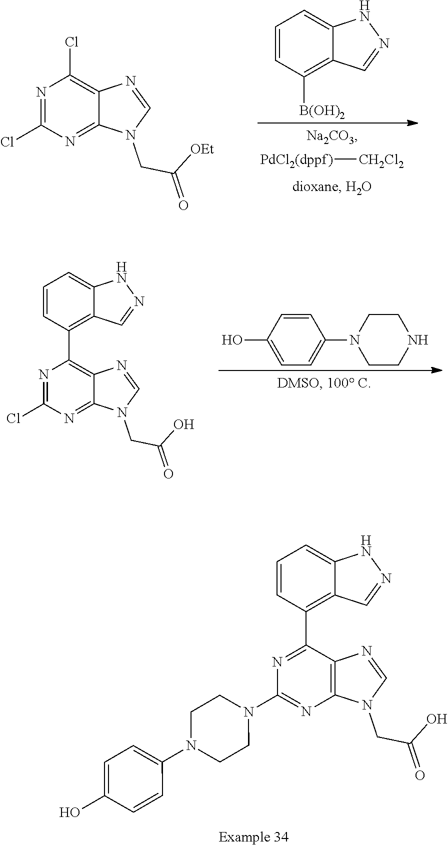

### Step 1:

A mixture of ethyl 2-(2,6-dichloro-9H-purin-9-yl)acetate (200 mg, 0.73 mmol, 1.0 eq), (1H-indazol-4-yl)boronic acid (130 mg, 0.80 mmol, 1.1 eq), Na₂CO₃ (230 mg, 2.18 mmol, 3 eq), PdCl2 (dppf)—CH2Cl2 (60 mg, 0.073 mmol, 0.1 eq) in dioxane (4 mL) and H₂O (1 mL) under a nitrogen atmosphere was stirred overnight at 100° C. The reaction mixture was allowed to cool and diluted with water (5 mL) and extracted with EtOAc (50 mL). The organic layer was washed with brine, dried over Na₂SO₄, and concentrated. The residue was purified by flash column chromatography on silica gel (1-10% of MeOH in DCM) to give 2-(2-chloro-6-(1H-indazol-4-yl)-9H-purin-9-yl)acetic acid (200 mg, 36% yield) as a white solid.

### Step 2:

A mixture of 2-(2-chloro-6-(1H-indazol-4-yl)-9H-purin-9-yl)acetic acid (160 mg, 0.49 mmol, 1.0 eq) and 1-(4-hydroxyphenyl) piperazine (261 mg, 1.46 mmol, 3.0 eq) in DMSO (5 mL) under a nitrogen atmosphere was stirred at 100° C. overnight. The reaction was allowed to cool and diluted with water (10 mL). The resulting precipitate was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(1H-indazol-4-yl)-9H-purin-9-yl)acetic acid (15 mg, 23% yield) as a solid.

¹H NMR (400 MHz, MeOD-d4): δ 8.70 (s, 1H), 8.53 (d, J=7.2 Hz, 1H), 8.27 (s, 1H), 7.78 (d, J=8.4 Hz, 1H), 7.64-7.59 (m, 1H), 7.52 (d, J=8.8 Hz, 2H), 6.97 (d, J=8.8 Hz, 2H), 5.12 (s, 2H), 4.53-4.28 (m, 4H), 3.85-3.78 (m, 4H); LC/MS(ESI-MS): [M+H]+=471.2.

### Example 35. 2-(2-(4-(4-hydroxyphenyl)piperazin-1-yl)-6-(1H-pyrrolo[2,3-b]pyridin-4-yl)-9H-purin-9-yl)acetic acid

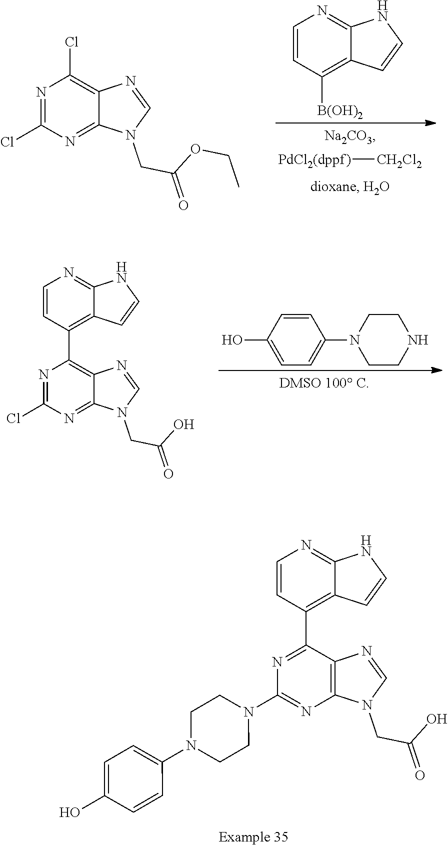

The proceeding example was prepared according to Example 34 substituting (1H-indazol-4-yl)boronic acid with (1H-pyrrolo[2,3-b]pyridin-4-yl)boronic acid. The desired product 2-(2-(4-(4-hydroxyphenyl)piperazin-1-yl)-6-(1H-pyrrolo[2,3-b]pyridin-4-yl)-9H-purin-9-yl)acetic acid was isolated as a white solid.

¹H NMR (400 MHz, DMSO-d6): δ 11.92 (s, 1H), 8.79 (s, 1H), 8.52 (d, J=4.8 Hz, 1H), 8.43 (d, J=5.2 Hz, 1H), 8.31-8.27 (m, 1H), 7.84-7.74 (m, 1H), 7.66 (s, 1H), 7.19-7.15 (m, 3H), 6.79-6.76 (m, 2H), 5.03 (s, 2H), 4.21-4.02 (m, 4H), 3.37-3.31 (m, 4H); LC/MS(ESI-MS): [M+H]+=471.5.

### Example 36. 2-(2-(4-(4-hydroxyphenyl)piperazin-1-yl)-6-(3-(pyridin-3-yl)-1H-indazol-1-yl)-9H-purin-9-yl)acetic acid

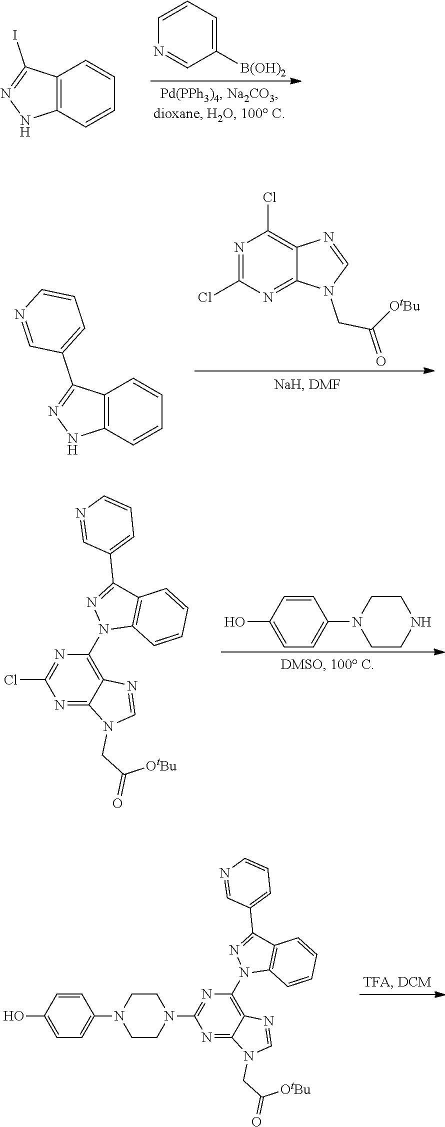

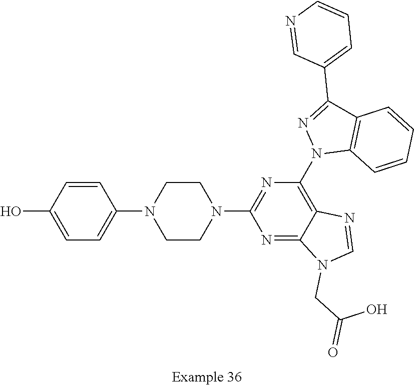 [SMILES: O=C(O)Cn1cnc2c(-n3nc(-c4cccnc4)c4ccccc43)nc(N3CCN(c4ccc(O)cc4)CC3)nc21]

### Step 1:

A mixture of 3-iodo-1H-indazole (1.5 g, 6.15 mmol, 1.0 eq), pyridin-3-ylboronic acid (982 mg, 7.99 mmol, 1.3 eq), Pd(PPh3)4 (1.07 g, 0.92 mmol, 0.15 eq) and Na₂CO₃ (1.63 g, 15.37 mmol, 2.5 eq) in dioxane (30 mL) and water (3 mL) under a nitrogen atmosphere was heated to reflux for overnight. The reaction was allowed to cool to room temperature and the solid was filtered off. The filtrate was partitioned between EtOAc (100 mL) and water (50 mL). The organic layer was washed with brine, dried over Na₂SO₄ and concentrated. The residue was purified by flash column chromatography on silica gel (40% EtOAc in hexane) to provide 3-(pyridin-3-yl)-1H-indazole (1.40 g) as a solid.

### Step 2:

To a solution of 3-(pyridin-3-yl)-1H-indazole (300 mg, 1.53 mmol, 1.0 eq) in DMF (8 mL) under a N2 atmosphere at 0° C. was added NaH (74 mg, 1.84 mmol, 60% in mineral oil, 1.2 eq). The mixture was stirred for 30 min and followed by addition of tert-butyl 2-(2,6-dichloro-9H-purin-9-yl)acetate (466 mg, 1.53 mmol, 1.0 eq). The mixture was stirred for 1 h at room temperature before it was diluted with water (10 mL). The precipitate was collected by filtration and further purified by re-crystallization in MeOH to give tert-butyl 2-(2-chloro-6-(3-(pyridin-3-yl)-1H-indazol-1-yl)-9H-purin-9-yl)acetate (300 mg, 42% yield) as an off-white solid.

### Step 3:

A mixture of tert-butyl 2-(2-chloro-6-(3-(pyridin-3-yl)-1H-indazol-1-yl)-9H-purin-9-yl)acetate (150 mg, 0.32 mmol, 1.0 eq) and 4-(piperazin-1-yl)phenol (145 mg, 0.81 mmol, 2.5 eq) in DMSO (5 mL) under a nitrogen atmosphere was stirred for overnight at 100° C. The reaction was allowed to cool to room temperature and diluted with water (10 mL). The precipitate was collected by filtration and dried to give tert-butyl 2-(2-(4-(4-hydroxyphenyl)-piperazin-1-yl)-6-(3-(pyridin-3-yl)-1H-indazol-1-yl)-9H-purin-9-yl)acetate (170 mg, 87% yield) as a light yellow solid.

### Step 4:

A mixture of tert-butyl 2-(2-(4-(4-hydroxyphenyl)-piperazin-1-yl)-6-(3-(pyridin-3-yl)-1H-indazol-1-yl)-9H-purin-9-yl)acetate (170 mg, 0.282 mmol, 1.0 eq) and TFA (2 mL) in DCM (3 mL) was stirred for 2 hours at room temperature before the solvent was removed under reduced pressure. The residue was purified by prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(3-(pyridin-3-yl)-1H-indazol-1-yl)-9H-purin-9-yl)acetic acid (140 mg, 91% yield) as a solid.

¹H NMR (400 MHz, DMSO-d6): δ 9.38 (s, 1H), 8.82 (s, 1H), 8.69-8.57 (m, 3H), 8.28 (d, J=8.4 Hz, 1H), 7.79-7.73 (m, 2H), 7.56-7.52 (m, 1H), 7.25-7.18 (m, 2H), 6.82-6.80 (m, 2H), 5.08 (s, 2H), 4.23-4.06 (m, 4H), 3.45 (br, 4H); LC/MS(ESI-MS): [M+H]+=548.2.

### Example 37. 2-chloro-3-(2-(4-(4-hydroxyphenyl)piperazin-1-yl)-6-methyl-9H-purin-9-yl)phenol

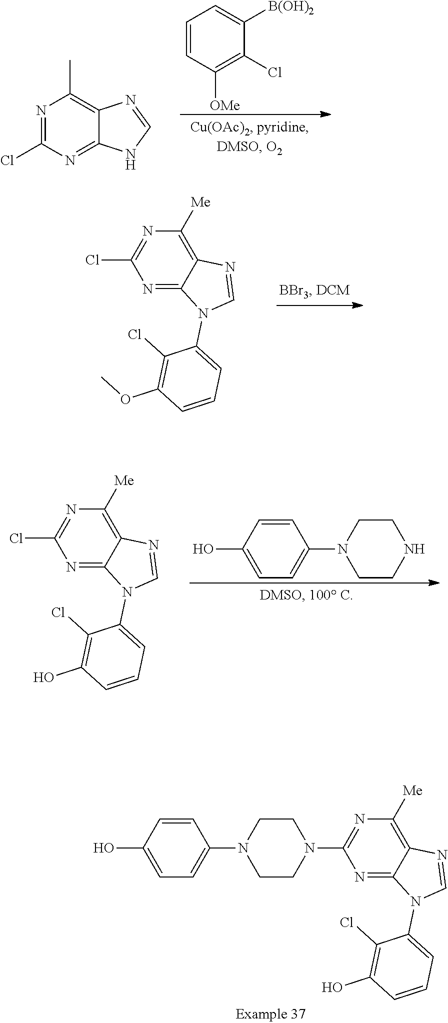 [SMILES: Cc1nc(Cl)nc2[nH]cnc12 ; COc1cccc(B(O)O)c1Cl ; COc1cccc(-n2cnc3c(C)nc(Cl)nc32)c1Cl ; Cc1nc(Cl)nc2c1ncn2-c1cccc(O)c1Cl ; Oc1ccc(N2CCNCC2)cc1 ; Cc1nc(N2CCN(c3ccc(O)cc3)CC2)nc2c1ncn2-c1cccc(O)c1Cl]

### Step 1:

To a solution of 2-chloro-6-methyl-9H-purine (0.3 g, 1.5 mmol, 1.0 eq) in DMSO (10 mL) at 25° C. under an oxygen atmosphere (1 atm balloon) was added (2-chloro-3-methoxyphenyl)boronic acid (0.82 g, 4.4 mmol, 3 eq), pyridine (0.35 g, 4.4 mmol, 3 eq), anhydrous cupric acetate (0.58 g, 2.9 mmol, 2 eq). The reaction mixture was then stirred for 3 days before it was diluted with water (20 mL) and NH₄OH (1 mL). The resulting solid was collected by filtration to give crude 2-chloro-9-(2-chloro-3-methoxyphenyl)-6-methyl-9H-purine (0.25 g, 54% yield).

### Step 2:

To a solution of crude 2-chloro-9-(2-chloro-3-methoxyphenyl)-6-methyl-9H-purine (250 mg, 0.81 mmol, 1.0 eq) in DCM (5 mL) at 0° C. was added BBr3 (3.2 g, 12.86 mmol, 20 eq) dropwise. The mixture was stirred at 25° C. overnight before it was diluted with water (30 mL). The resulting precipitate was collected by filtration and dried to give crude 2-chloro-3-(2-chloro-6-methyl-9H-purin-9-yl)phenol (0.2 g, 83% yield) as a white solid.

### Step 3:

A mixture of crude 2-chloro-3-(2-chloro-6-methyl-9H-purin-9-yl)phenol (0.2 g, 0.67 mmol, 1.0 eq) and 1-(4-hydroxyphenyl) piperazine (0.59 g, 3.35 mmol, 5.0 eq) in DMSO (5 mL) under a nitrogen atmosphere was stirred overnight at 100° C. The reaction was allowed to cool and diluted with water (20 mL). The resulting solid was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-chloro-3-(2-(4-(4-hydroxyphenyl)piperazin-1-yl)-6-methyl-9H-purin-9-yl)phenol (50 mg, 17% yield).

¹H NMR (400 MHz, DMSO-d6): δ 10.78 (br, 1H), 9.62 (br, 1H), 8.27 (s, 1H), 7.37-7.18 (m, 4H), 7.08 (d, J=1.2 Hz, 1H), 6.82-6.70 (m, 2H), 4.05-3.63 (m, 4H), 3.42-3.17 (m, 4H), 2.64 (s, 3H); LC/MS(ESI-MS): [M+H]+=437.1.

### Example 38. 4-(4-(6-methyl-9-(5-methylpyridin-3-yl)-9H-purin-2-yl)piperazin-1-yl)phenol

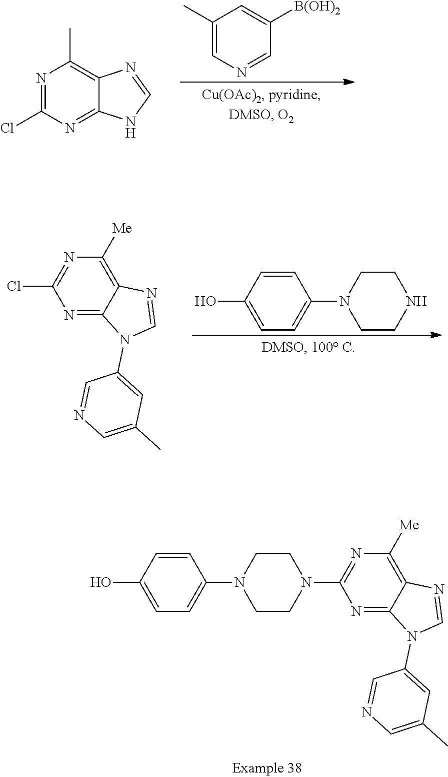

The proceeding example was prepared according to Example 37 substituting (2-chloro-3-methoxyphenyl)boronic acid with (5-methylpyridin-3-yl)boronic acid. The desired product 4-(4-(6-methyl-9-(5-methylpyridin-3-yl)-9H-purin-2-yl)piperazin-1-yl)phenol was isolated as a white solid.

¹H NMR (400 MHz, DMSO-d6): δ 9.02 (s, 1H), 8.86 (s, 1H), 8.59 (s, 1H), 8.49 (s, 1H), 8.18 (s, 1H), 6.85 (d, J=8.8 Hz, 2H), 6.67 (d, J=8.8 Hz, 2H), 3.93-3.88 (m, 4H), 3.05-3.02 (m, 4H); LC/MS(ESI-MS): [M+H]+=402.1.

### Example 39. 4-(4-(6-(indolin-1-yl)-9-(pyridin-3-yl)-9H-purin-2-yl)piperazin-1-yl)phenol

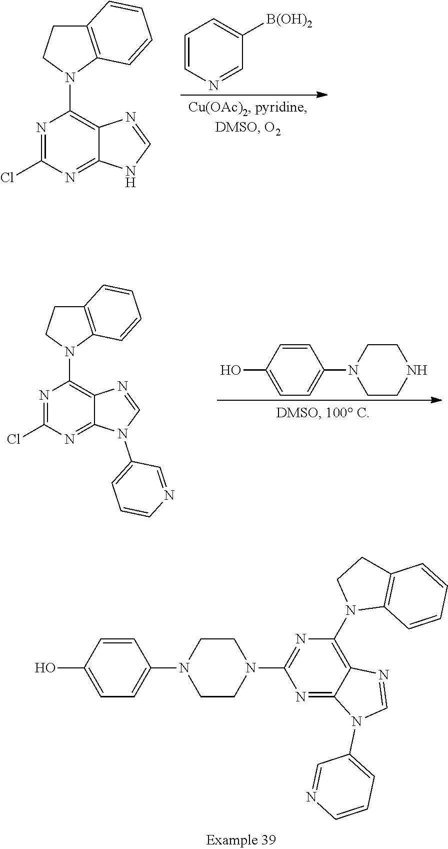

### Step 1:

To a solution of 2-chloro-6-(indolin-1-yl)-9H-purine (0.3 g, 1.10 mmol, 1.0 eq) in DMSO (10 mL) at 25° C. was added pyridin-3-ylboronic acid (0.27 g, 2.21 mmol, 2.0 eq), pyridine (0.26 g, 3.31 mmol, 2.0 eq) and anhydrous cupric acetate (0.44 g, 2.21 mmol, 2.0 eq). The reaction mixture was stirred at room temperature for 3 days before it was diluted with water (20 mL) and of NH₃.H₂O (1 mL). The resulting precipitate was collected by filtration and dried to give crude 2-chloro-6-(indolin-1-yl)-9-(pyridin-3-yl)-9H-purine (0.15 g, 39% yield).

### Step 2:

A mixture of crude 2-chloro-6-(indolin-1-yl)-9-(pyridin-3-yl)-9H-purine (0.15 g, 0.43 mmol, 1.0 eq) and 1-(4-hydroxyphenyl) piperazine (0.23 g, 1.29 mmol, 3.0 eq) in DMSO (5 mL) under a nitrogen atmosphere was stirred overnight at 100° C. The reaction was allowed to cool and diluted with water (10 mL). The resulting solid was collected by filtration and further purified by Prep-HPLC (Column. Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(6-(indolin-1-yl)-9-(pyridin-3-yl)-9H-purin-2-yl)piperazin-1-yl)phenol (55 mg, 26% yield).

¹H NMR (400 MHz, DMSO-d6): δ 9.18 (s, 1H), 8.85 (s, 1H), 8.63 (d, J=4.8 Hz, 1H), 8.47 (s, 1H), 8.39-8.36 (m, 2H), 7.67-7.64 (m, 1H), 7.32-7.23 (m, 2H), 7.00 (d, J=7.2 Hz, 1H), 6.86 (d, J=8.8 Hz, 2H), 6.67 (d, J=8.8 Hz, 1H), 4.76 (d, J=8.4 Hz, 2H), 3.94-3.83 (m, 4H), 3.30-3.21 (m, 2H), 3.13-3.05 (m, 4H); LC/MS(ESI-MS): [M+H]+=491.2.

### Example 40. ethyl 2-(6-(1H-benzo[d]imidazol-1-yl)-2-(4-(4-hydroxyphenyl)piperazin-1-yl)-9H-purin-9-yl)acetate

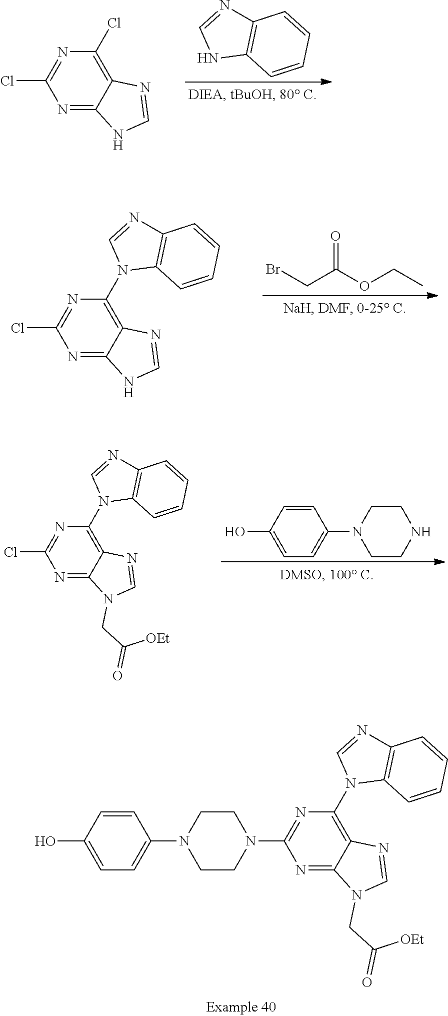

### Step 1:

A mixture of 2,6-dichloropurine (5 g, 26.46 mmol, 1.0 eq), indazole (6.25 g, 52.91 mmol, 2.0 eq) and DIEA (10.26 g, 79.37 mmol, 3 eq) in t-BuOH (50 mL) was stirred overnight at 80° C. The reaction was allowed to cool and the solvent was removed. The residue was partitioned between water (50 mL) and EtOAc (150 mL). The organic layer was washed with brine, dried over Na₂SO₄, concentrated. The residue was purified by flash column chromatography (DCM:MeOH=10:1) to give 6-(1H-benzo[d]imidazol-1-yl)-2-chloro-9H-purine (2.3 g, 33% yield) as a white solid.

### Step 2:

To a solution of 6-(1H-benzo[d]imidazol-1-yl)-2-chloro-9H-purine (2.30 g, 8.50 mmol, 1.0 eq) in DMF (5 mL) at 0° C. was added NaH (408 mg, 10.20 mmol, 1.2 eq, 60% in oil). The mixture was stirred for 30 min at 0° C. and followed by addition of ethyl bromoacetate (1.84 g, 11.05 mmol, 1.3 eq). The mixture was then stirred from 0-25° C. over 4 h before it was quenched with 1N aq. HCl and extracted with EtOAc (100 mL). The organic layer was washed with water (2×50 mL), brine (20 mL), dried (Na₂SO₄) and concentrated. The crude residue was purified by flash column chromatography on silica gel (10-70% EtOAc in petroleum ether) to provide ethyl 2-(6-(1H-benzo[d]imidazol-1-yl)-2-chloro-9H-purin-9-yl)acetate (1.60 g) as a white solid.

### Step 3:

To a solution of ethyl 2-(6-(1H-benzo[d]imidazol-1-yl)-2-chloro-9H-purin-9-yl)acetate (1.6 g, 4.48 mmol, 1.0 eq) in 15 mL of DMSO, 1-(4-hydroxyphenyl) piperazine (2.4 g, 13.45 mmol, 3.0 eq) was added and the mixture was stirred overnight at 100° C. under nitrogen atmosphere. The reaction mixture was allowed to cool and diluted with water (6 mL). The resulting solid precipitate was collected by filtration. The crude solid was purified by Prep-HPLC (Column. Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give ethyl 2-(6-(1H-benzo[d]imidazol-1-yl)-2-(4-(4-hydroxyphenyl)piperazin-1-yl)-9H-purin-9-yl)acetate (380 mg, 17% yield) as a white solid.

¹H NMR (300 MHz, DMSO-d6): δ 9.76 (s, 1H), 8.57 (d, J=7.8 Hz, 1H), 8.35 (s, 1H), 7.84 (d, J=7.2 Hz, 1H), 7.51-7.40 (m, 2H), 7.18-7.08 (m, 2H), 6.77 (d, J=8.4 Hz, 2H), 5.14 (s, 2H), 4.25-4.10 (m, 6H), 3.43-3.26 (m, 4H), 1.25 (t, J=7.2 Hz, 3H); LC/MS(ESI-MS): [M+H]+=499.1.

### Example 41. 2-(6-(1H-benzo[d]imidazol-1-yl)-2-(4-(4-hydroxyphenyl)piperazin-1-yl)-9H-purin-9-yl)acetic acid

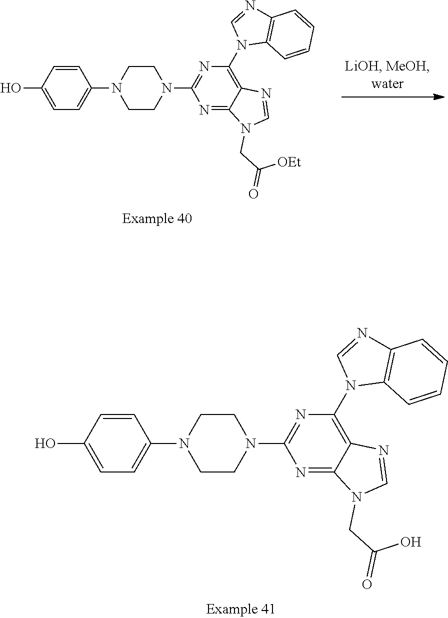 [SMILES: CCOC(=O)Cn1cnc2c(-n3cnc4ccccc43)nc(N3CCN(c4ccc(O)cc4)CC3)nc21 ; O=C(O)Cn1cnc2c(-n3cnc4ccccc43)nc(N3CCN(c4ccc(O)cc4)CC3)nc21]

To a solution of ethyl 2-(6-(1H-benzo[d]imidazol-1-yl)-2-(4-(4-hydroxyphenyl)piper-azin-1-yl)-9H-purin-9-yl)acetate (100 mg, 0.20 mmol, 1.0 eq) in a mixture of H₂O (3 mL) and MeOH (3 mL) was added LiOH (42 mg, 1.01 mmol, 5 eq). The mixture was stirred at room temperature for 3 h before the solvent was removed under reduced pressure. The residue was re-dissolved in water (5 mL) and adjusted its pH to 5 with 2N aq. HCl solution. The resulting precipitate was collected by filtration. The crude solid was purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(6-(1H-benzo[d]imidazol-1-yl)-2-(4-(4-hydroxyphenyl)piperazin-1-yl)-9H-purin-9-yl)acetic acid (55 mg, 58% yield) as a white solid.

¹H NMR (400 MHz, DMSO-d6): 13.42 (br, 1H), 9.75 (s, 1H), 8.88 (s, 1H), 8.60 (s, 1H), 8.30 (s, 1H), 7.84 (d, J=7.6 Hz, 1H), 7.52-7.40 (m, 2H), 6.88 (d, J=8.8 Hz, 2H), 6.69 (d, J=8.8 Hz, 2H), 5.01 (s, 2H), 4.03-3.91 (m, 4H), 3.20-3.01 (m, 4H); LC/MS(ESI-MS): [M+H]+=471.2.

### Example 42. 2-(6-(1H-benzo[d]imidazol-1-yl)-2-(4-(4-hydroxyphenyl)piperazin-1-yl)-9H-purin-9-yl)-N-methylacetamide

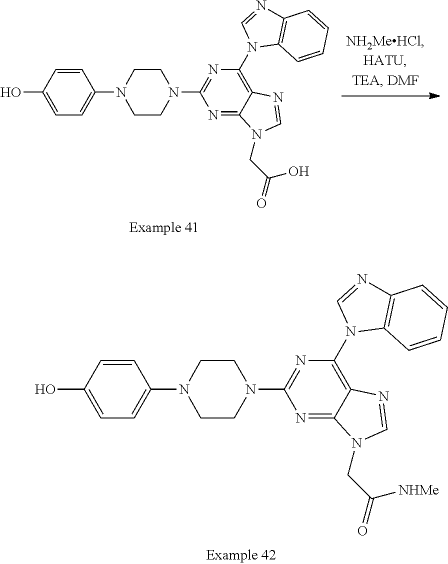 [SMILES: O=C(O)Cn1cnc2c(-n3cnc4ccccc43)nc(N3CCN(c4ccc(O)cc4)CC3)nc21 ; CNC(=O)Cn1cnc2c(-n3cnc4ccccc43)nc(N3CCN(c4ccc(O)cc4)CC3)nc21]

To a solution of 2-(6-(1H-benzo[d]imidazol-1-yl)-2-(4-(4-hydroxyphenyl)piperazin-1-yl)-9H-purin-9-yl)acetic acid (0.21 g, 0.46 mmol, 1.0 eq) in DMF (6 mL) at 25° C. was added HATU (0.21 g, 0.55 mmol, 1.2 eq). The mixture was stirred for 0.5 h and followed bt addition of Et3N (93 mg, 0.92 mmol, 2 eq) and methylamine hydrochloride (93 mg, 1.38 mmol, 3 eq). The mixture was stirred overnight before it was diluted with water (15 mL). The resulting precipitate was collected by filtration and purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(6-(1H-benzo[d]imidazol-1-yl)-2-(4-(4-hydroxyphenyl)piperazin-1-yl)-9H-purin-9-yl)-N-methylacetamide (102 mg, 11% yield) as a white solid.

¹H NMR (400 MHz, DMSO-d6): δ 9.77 (s, 1H), 8.58 (d, J=8.0 Hz, 1H), 8.30 (s, 1H), 8.26-8.24 (m, 1H), 7.84 (d, J=7.6 Hz, 1H), 7.51-7.40 (m, 2H), 7.12-6.98 (m, 2H), 6.76 (d, J=8.4 Hz, 2H), 4.88 (s, 2H), 4.23-4.08 (m, 4H), 3.44-3.30 (m, 4H), 2.67 (s, 3H); LC/MS(ESI-MS): [M+H]+=484.2.

### Example 43. 4-(4-(6-methyl-9-phenyl-9H-purin-2-yl)piperazin-1-yl)phenol

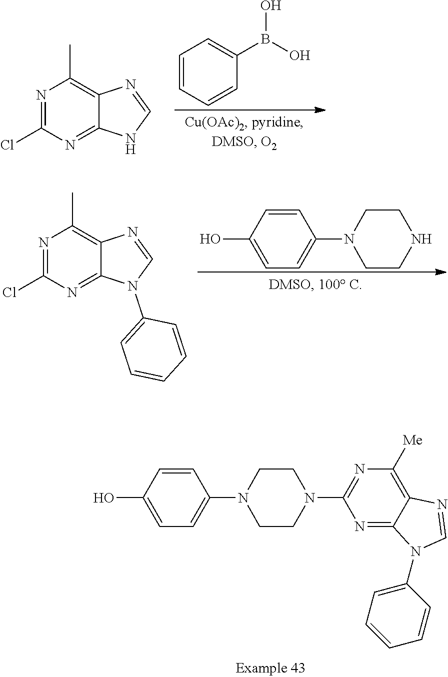

The proceeding example was prepared according to Example 37 substituting (2-chloro-3-methoxyphenyl)boronic acid with phenylboronic acid. The desired product 4-(4-(6-methyl-9-phenyl-9H-purin-2-yl)piperazin-1-yl)phenol was isolated as a white solid.

¹H NMR (400 MHz, DMSO-d6): δ 8.87 (s, 1H), 8.55 (s, 1H), 7.92 (d, J=7.2 Hz, 2H), 7.61 (t, J=8.4 Hz, 1H), 7.44 (t, J=7.2 Hz, 1H), 6.85 (d, J=7.2 Hz, 2H), 6.67 (d, J=8.8 Hz, 2H), 3.90-3.87 (m, 4H), 3.04-3.02 (m, 4H), 2.62 (s, 3H); LC/MS(ESI-MS): [M+H]+=387.1.

### Example 44. ethyl 2-(2-(4-(4-hydroxyphenyl)piperazin-1-yl)-6-(1H-indazol-1-yl)-9H-purin-9-yl)acetate

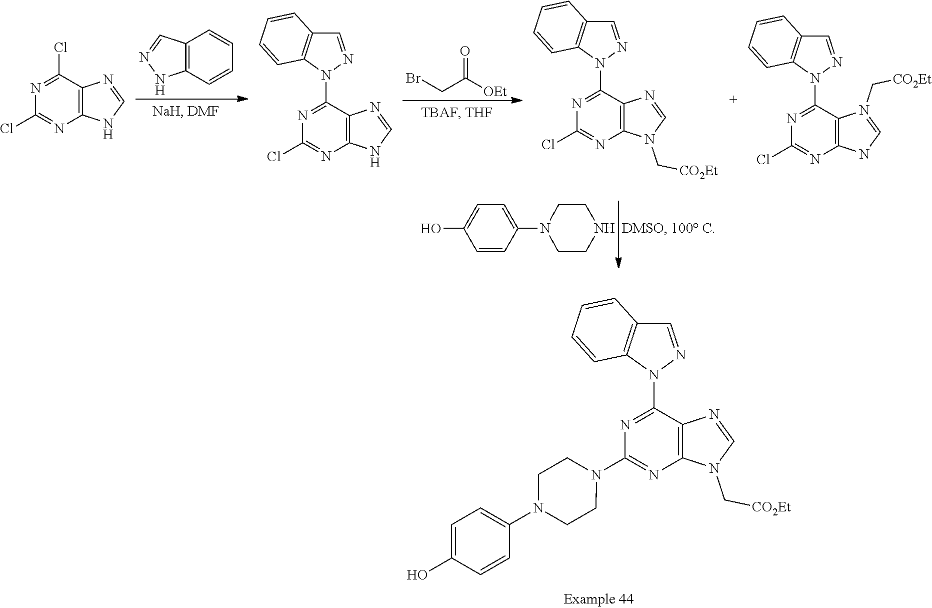

### Step 1:

To a solution of indazole (625 mg, 5.29 mmol, 1.0 eq) in DMF (10 mL) at 25° C. was added NaH (423 mg, 10.58 mmol, 2.0 eq). The mixture was stirred at room temperature for 30 min and followed by addition of a solution of 2,6-dichloro-9H-purine (1 g, 5.29 mmol, 1.0 eq) in DMF (4 mL) dropwise. The resulting mixture was then heated at 80° C. and stirred for overnight. The reaction was allowed to cool and diluted with water (5 mL) and MeCN (10 mL). The resulting precipitate was collected by filtration and dried to 2-chloro-6-(1H-indazol-1-yl)-9H-purine (900 mg, 70% yield) as a white solid.

### Step 2:

The solution of 2-chloro-6-(1H-indazol-1-yl)-9H-purine (3 g, 11.11 mmol, 1.0 eq) in THF (30 mL) under a N2 atmosphere at 0° C. was added a solution of 1M TBAF in THF (22.17 mL, 22.17 mmol, 2 eq). The reaction mixture was stirred for 15 min followed by addition of ethyl 2-bromoacetate (2.78 g, 16.62 mmol, 1.5 eq). The resulting mixture was stirred from 0-25° C. over a period of 3 h before it was quenched with saturated aq. NH₄Cl (100 mL) and extracted with EtOAc (150 mL). The organic layer was washed with brine, dried over Na₂SO₄ and concentrated. The crude residue was purified by flash column chromatography on silica gel (1-70% of EtOAc in petroleum ether) to provide ethyl 2-(2-chloro-6-(1H-indazol-1-yl)-9H-purin-9-yl)acetate (2.12 g) and ethyl 2-(2-chloro-6-(1H-indazol-1-yl)-7H-purin-7-yl)acetate (0.75 g) as both white solids.

### Step 3:

A mixture of ethyl 2-(2-chloro-6-(1H-indazol-1-yl)-9H-purin-9-yl)acetate (1 g, 2.8 mmol, 1.0 eq) and 4-(piperazin-1-yl)phenol (2.5 g, 14.0 mmol, 5.0 eq) in DMSO (15 mL) was stirred at 100° C. overnight under nitrogen atmosphere. The reaction mixture was allowed to cool and diluted with water (50 mL). The resulting solid precipitate was collected by filtration, rinsed with water (2 mL) and dried. This crude product was further purified by Prep-HPLC to give ethyl 2-(2-(4-(4-hydroxyphenyl)pipera-zin-1-yl)-6-(1H-indazol-1-yl)-9H-purin-9-yl)acetate (850 mg) as a white solid.

¹H NMR (400 MHz, DMSO-d6): δ 8.87 (s, 1H), 8.63-8.58 (m, 2H), 8.18 (s, 1H), 7.95 (d, J=8.0 Hz, 1H), 7.04 (t, J=7.6 Hz, 1H), 7.40 (t, J=7.6 Hz, 1H), 6.89 (d, J=8.4 Hz, 2H), 6.69 (d, J=8.4 Hz, 2H), 5.09 (s, 2H), 4.21 (q, J=6.8 Hz, 2H), 4.01-3.92 (m, 4H), 3.13-3.06 (m, 4H), 1.24 (t, J=6.8 Hz, 3H); LC/MS(ESI-MS): [M+H]+=499.2.

### Example 45. 2-(2-(4-(4-hydroxyphenyl)piperazin-1-yl)-6-(1H-indazol-1-yl)-9H-purin-9-yl)acetic acid

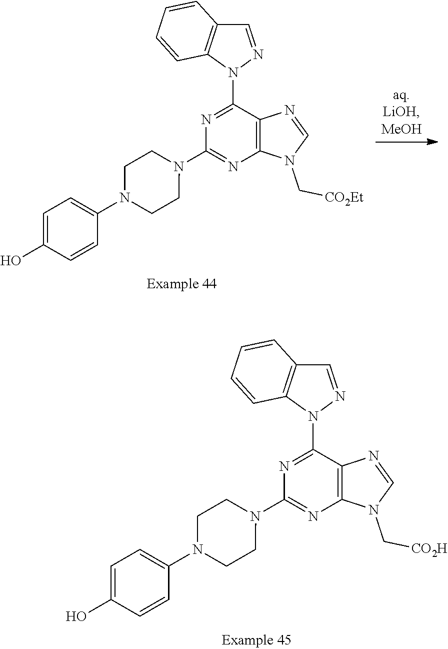 [SMILES: CCOC(=O)Cn1cnc2c(-n3ncc4ccccc43)nc(N3CCN(c4ccc(O)cc4)CC3)nc21 ; O=C(O)Cn1cnc2c(-n3ncc4ccccc43)nc(N3CCN(c4ccc(O)cc4)CC3)nc21]

To a solution of ethyl 2-(2-(4-(4-hydroxyphenyl)piperazin-1-yl)-6-(1H-indazol-1-yl)-9H-purin-9-yl)acetate (1.0 g, 2.00 mmol, 1.0 eq) in a mixture of H₂O (3 mL) and MeOH (3 mL) was added LiOH (252 mg, 6.02 mmol, 3.0 eq). The mixture was stirred at room temperature for 3 h before it was concentrated under reduced pressure. The residue was re-dissolved in water (15 mL) and its pH was adjust to 5 with 2 N aq. HCl solution. The resulting precipitate was collected by filtration. The solid was dried to give 1-(9-(carboxymethyl)-2-(4-(4-hydroxyphenyl)piperazin-1-yl)-9H-purin-6-yl)-1H-indazole-3-carboxylic acid (670 mg) as a white solid.

¹H NMR (400 MHz, DMSO-d6): δ 8.96 (s, 1H), 8.62-8.56 (m, 2H), 8.05 (s, 1H), 7.94 (d, J=8 Hz, 1H), 7.62 (t, J=8 Hz, 1H), 7.38 (t, J=8 Hz, 1H), 6.88 (d, J=9 Hz, 2H), 6.68 (d, J=9 Hz, 2H), 4.41 (s, 2H), 3.97 (bs, 4H), 3.12 (bs, 4H); LC/MS(ESI-MS): [M+H]+=471.1.

### Example 46. 2-(2-(4-(4-hydroxyphenyl)piperazin-1-yl)-6-(1H-indazol-1-yl)-7H-purin-7-yl)acetic acid

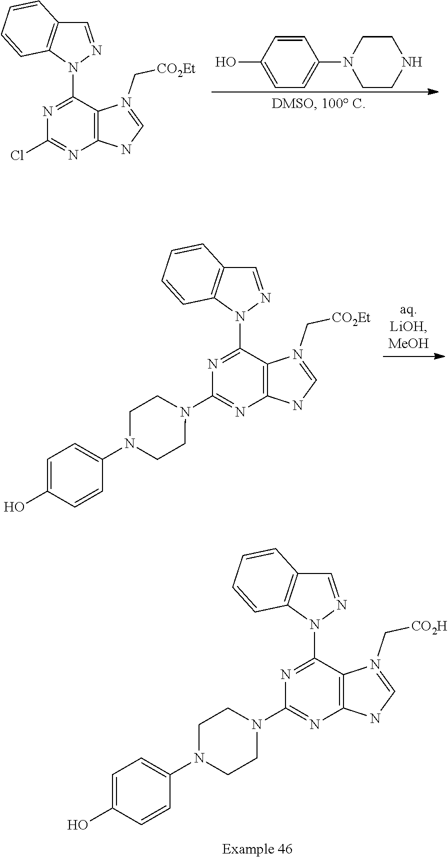

The proceeding example was prepared according to Examples 44 and 45 substituting ethyl 2-(2-chloro-6-(1H-indazol-1-yl)-9H-purin-9-yl)acetate with ethyl 2-(2-chloro-6-(1H-indazol-1-yl)-7H-purin-7-yl)acetate. The desired product 2-(2-(4-(4-hydroxyphenyl)pipera-zin-1-yl)-6-(1H-indazol-1-yl)-7H-purin-7-yl)acetic acid was isolated as a white solid. ¹H NMR (400 MHz, DMSO-d6): δ 9.42 (s, 1H), 8.56 (s, 1H), 7.83 (d, J=8 Hz, 1H), 7.71 (d, J=8 Hz, 1H), 7.41 (t, J=7 Hz, 1H), 7.19-7.16 (m, 3H), 7.38 (t, J=8 Hz, 1H), 6.78 (d, J=7 Hz, 2H), 5.52 (s, 2H), 4.08 (bs, 4H), 3.35 (bs, 4H); LC/MS(ESI-MS): [M+H]+=471.2.

### Example 47. 2-(2-(4-(4-hydroxyphenyl)piperazin-1-yl)-6-(1H-indazol-1-yl)-9H-purin-9-yl)acetamide

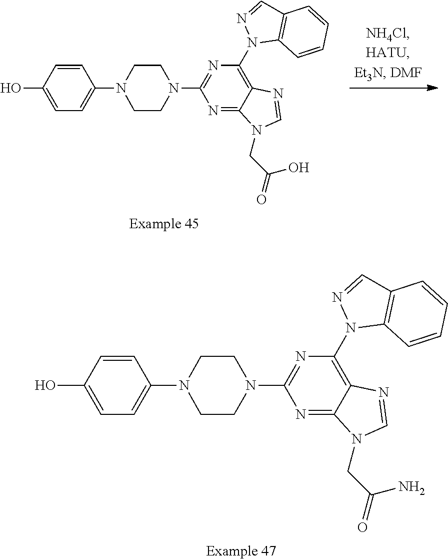 [SMILES: O=C(O)Cn1cnc2c(-n3ncc4ccccc43)nc(N3CCN(c4ccc(O)cc4)CC3)nc21 ; NC(=O)Cn1cnc2c(-n3ncc4ccccc43)nc(N3CCN(c4ccc(O)cc4)CC3)nc21]

A solution of 2-(2-(4-(4-hydroxyphenyl)piperazin-1-yl)-6-(1H-indazol-1-yl)-9H-purin-9-yl)acetic acid (300 mg, 0.64 mmol, 1.0 eq) and HATU (291 mg, 0.77 mmol, 1.2 eq) in DMF (5 mL) at room temperature was stirred for 1.5 hours and followed by addition of NH₄Cl (102 mg, 1.91 mmol, 3.0 eq) and Et₃N (129 mg, 1.28 mmol, 2.0 eq). The mixture was stirred at room temperature for overnight under a nitrogen atmosphere before it was diluted water (10 mL). The resulting precipitate was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(1H-indazol-1-yl)-9H-purin-9-yl)acetamide (61 mg) as an off-white solid.

¹H NMR (300 MHz, DMSO-d6): δ 8.79 (s, 1H), 8.70 (s, 1H), 8.63-8.55 (m, 1H), 7.98 (d, J=7.8 Hz, 1H), 7.86 (s, 1H), 7.69 (d, J=8.1 Hz, 1H), 7.48-7.43 (m, 2H), 7.15-7.10 (m, 2H), 6.77 (d, J=8.7 Hz, 2H), 4.93 (s, 2H), 4.19-4.03 (m, 4H), 3.38-3.31 (m, 4H); LC/MS(ESI-MS): [M+H]+=470.2.

### Example 48. 2-(6-(3a,7a-dihydro-1H-indazol-1-yl)-2-(4-phenylpiperazin-1-yl)-9H-purin-9-yl)acetic acid

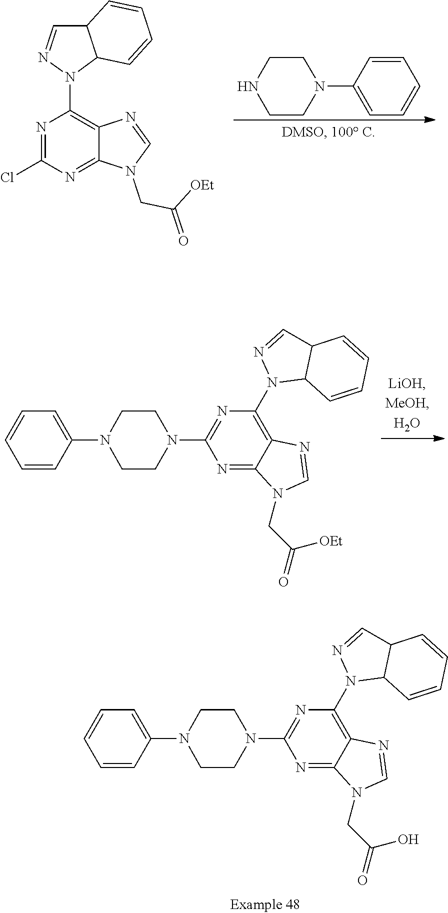 [SMILES: CCOC(=O)Cn1cnc2c(N3N=CC4C=CC=CC43)nc(Cl)nc21 ; c1ccc(N2CCNCC2)cc1 ; CCOC(=O)Cn1cnc2c(N3N=CC4C=CC=CC43)nc(N3CCN(c4ccccc4)CC3)nc21 ; O=C(O)Cn1cnc2c(N3N=CC4C=CC=CC43)nc(N3CCN(c4ccccc4)CC3)nc21]

The proceeding example was prepared according to Examples 44 and 45 substituting 4-(piperazin-1-yl)phenol with 1-phenylpiperazine. The desired product 2-(6-(3a,7a-dihydro-1H-indazol-1-yl)-2-(4-phenylpiperazin-1-yl)-9H-purin-9-yl)acetic acid was isolated as a white solid.

¹H NMR (400 MHz, CD3OD-d4): δ 9.14 (s, 1H), 8.76 (d, J=7.6 Hz, 1H), 8.61 (s, 1H), 7.99-7.93 (m, 1H), 7.76-7.69 (m, 1H), 7.52-7.48 (m, 1H), 7.34-7.31 (m, 2H), 7.14-7.10 (m, 2H), 6.97-6.93 (m, 1H), 5.23 (s, 2H), 4.24-4.17 (m, 4H), 3.53-3.42 (m, 4H); LC/MS(ESI-MS): [M+H]+=457.1.

### Example 49. 2-(6-(3a,7a-dihydro-1H-indazol-1-yl)-2-(4-(pyridin-4-yl)piperazin-1-yl)-9H-purin-9-yl)acetic acid

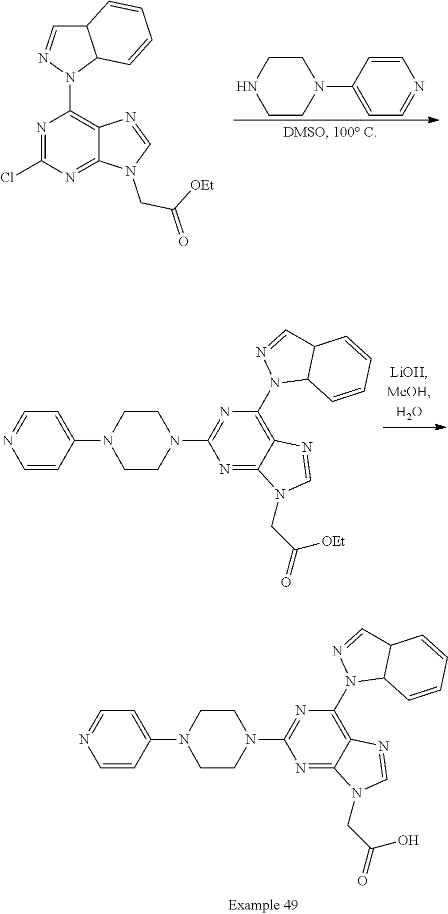 [SMILES: CCOC(=O)Cn1cnc2c(N3N=CC4C=CC=CC43)nc(Cl)nc21 ; c1cc(N2CCNCC2)ccn1 ; CCOC(=O)Cn1cnc2c(N3N=CC4C=CC=CC43)nc(N3CCN(c4ccncc4)CC3)nc21 ; O=C(O)Cn1cnc2c(N3N=CC4C=CC=CC43)nc(N3CCN(c4ccncc4)CC3)nc21]

The proceeding example was prepared according to Examples 44 and 45 substituting 4-(piperazin-1-yl)phenol with 1-(pyridin-4-yl)piperazine. The desired product 2-(6-(3a,7a-dihydro-1H-indazol-1-yl)-2-(4-(pyridin-4-yl)piperazin-1-yl)-9H-purin-9-yl)acetic acid was isolated as a white solid.

¹H NMR (400 MHz, MeOD-d4): δ 8.93 (br, 1H), 8.85-8.76 (m, 2H), 8.59 (d, J=7.6 Hz, 2H), 8.06-7.98 (m, 1H), 7.77-7.69 (m, 1H), 7.53-7.44 (m, 1H), 7.26 (d, J=7.6 Hz, 2H), 5.20 (s, 2H), 4.28-4.23 (m, 4H), 4.05-3.99 (m, 4H); LC/MS(ESI-MS): [M+H]+=458.1.

### Example 50. 2-(2-(4-(4-hydroxyphenyl)piperazin-1-yl)-6-(6-methoxy-1H-indazol-1-yl)-9H-purin-9-yl)acetic acid

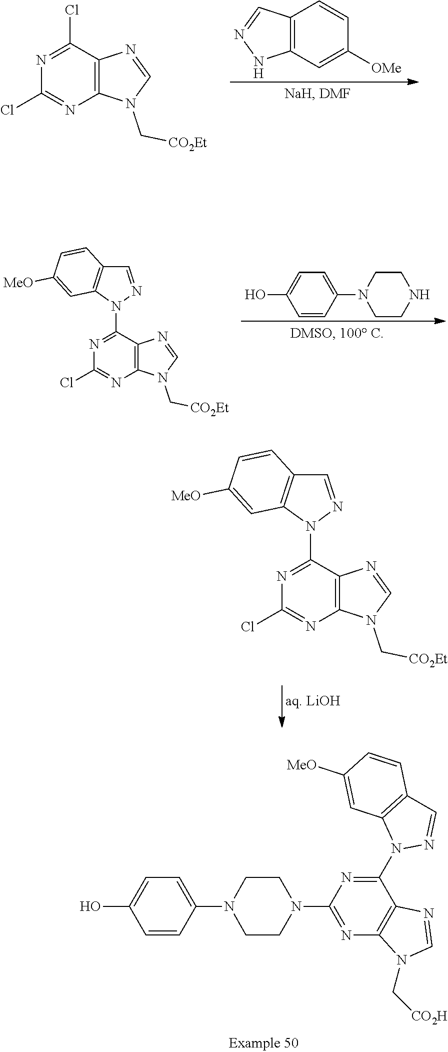 [SMILES: CCOC(=O)Cn1cnc2c(Cl)nc(Cl)nc21 ; COc1ccc2cn[nH]c2c1 ; CCOC(=O)Cn1cnc2c(-n3ncc4ccc(OC)cc43)nc(Cl)nc21 ; Oc1ccc(N2CCNCC2)cc1 ; CCOC(=O)Cn1cnc2c(-n3ncc4ccc(OC)cc43)nc(Cl)nc21 ; COc1ccc2cnn(-c3nc(N4CCN(c5ccc(O)cc5)CC4)nc4c3ncn4CC(=O)O)c2c1]

The proceeding example was prepared according to Example 11 substituting 3-(((tert-butyldimethylsilyl)oxy)methyl)-1H-indazole with 6-methoxy-1H-indazole. The desired product 2-(2-(4-(4-hydroxyphenyl)piperazin-1-yl)-6-(6-methoxy-1H-indazol-1-yl)-9H-purin-9-yl)acetic acid was isolated as a white solid.

¹H NMR (400 MHz, MeOD-d4): δ 8.85 (s, 1H), 8.30 (s, 1H), 8.04-7.97 (m, 1H), 7.70-7.62 (m, 1H), 7.24-7.21 (m, 2H), 6.95-6.91 (m, 1H), 6.79 (d, J=8.4 Hz, 2H), 5.07 (s, 2H), 4.23-4.17 (m, 4H), 3.88 (s, 3H), 3.50-3.43 (m, 4H); LC/MS(ESI-MS): [M+H]+=501.2.

### Example 51. 2-(2-(4-(4-hydroxyphenyl)piperazin-1-yl)-6-(5-methoxy-1H-indazol-1-yl)-9H-purin-9-yl)acetic acid

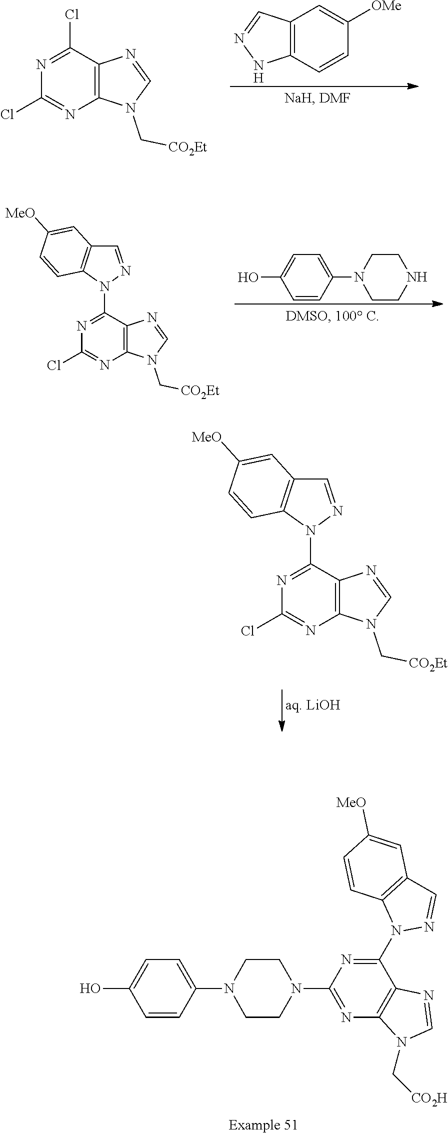 [SMILES: CCOC(=O)Cn1cnc2c(Cl)nc(Cl)nc21 ; COc1ccc2[nH]ncc2c1 ; CCOC(=O)Cn1cnc2c(-n3ncc4cc(OC)ccc43)nc(Cl)nc21 ; Oc1ccc(N2CCNCC2)cc1 ; CCOC(=O)Cn1cnc2c(-n3ncc4cc(OC)ccc43)nc(Cl)nc21 ; COc1ccc2c(cnn2-c2nc(N3CCN(c4ccc(O)cc4)CC3)nc3c2ncn3CC(=O)O)c1]

The proceeding example was prepared according to Example 11 substituting 3-(((tert-butyldimethylsilyl)oxy)methyl)-1H-indazole with 5-methoxy-1H-indazole. The desired product 2-(2-(4-(4-hydroxyphenyl)piperazin-1-yl)-6-(5-methoxy-1H-indazol-1-yl)-9H-purin-9-yl)acetic acid was isolated as a white solid.

¹H NMR (400 MHz, DMSO-d6): δ 8.53-8.51 (m, 2H), 8.41-8.37 (m, 1H), 7.41 (s, 1H), 7.31-7.28 (m, 1H), 7.07-6.97 (m, 1H), 6.75 (d, J=8.4 Hz, 2H), 5.03 (s, 2H), 4.11-3.97 (m, 4H), 3.87 (s, 3H), 3.24-3.14 (m, 4H); LC/MS(ESI-MS): [M+H]+=501.1.

### Example 52. 2-(6-(5-cyano-1H-indazol-1-yl)-2-(4-(4-hydroxyphenyl)piperazin-1-yl)-9H-purin-9-yl)acetic acid

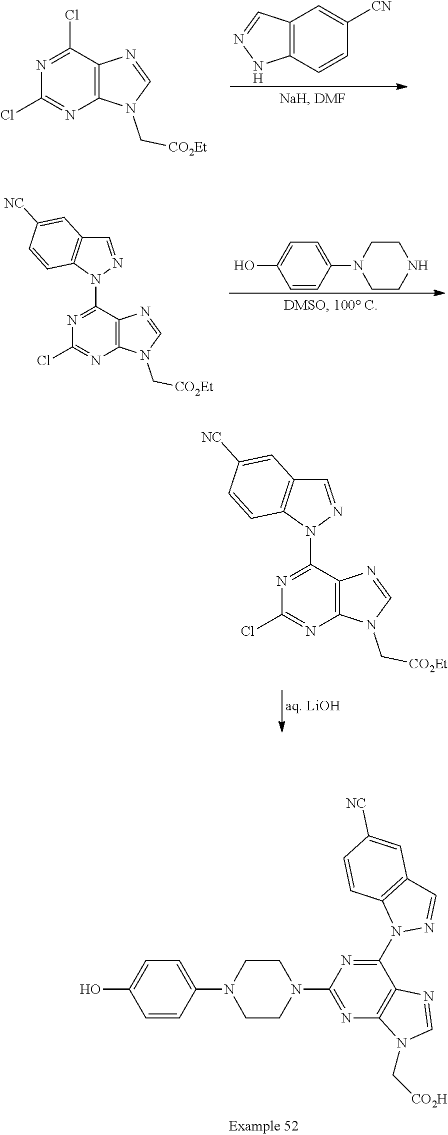 [SMILES: CCOC(=O)Cn1cnc2c(Cl)nc(Cl)nc21 ; N#Cc1ccc2[nH]ncc2c1 ; CCOC(=O)Cn1cnc2c(-n3ncc4cc(C#N)ccc43)nc(Cl)nc21 ; Oc1ccc(N2CCNCC2)cc1 ; CCOC(=O)Cn1cnc2c(-n3ncc4cc(C#N)ccc43)nc(Cl)nc21 ; N#Cc1ccc2c(cnn2-c2nc(N3CCN(c4ccc(O)cc4)CC3)nc3c2ncn3CC(=O)O)c1]

The proceeding example was prepared according to Example 1 substituting 3-(((tert-butyldimethylsilyl)oxy)methyl)-1H-indazole with 5-cyano-1H-indazole. The desired product 2-(2-(4-(4-hydroxyphenyl)piperazin-1-yl)-6-(5-cyano-1H-indazol-1-yl)-9H-purin-9-yl)acetic acid was isolated as a white solid.

¹H NMR (400 MHz, DMSO-d6): δ 8.76 (s, 1H), 8.73-8.62 (m, 1H), 8.58 (d, J=2.7 Hz, 1H), 8.37 (d, J=16.2 Hz, 1H), 8.02-7.91 (m, 1H), 7.25-7.05 (m, 2H), 6.78 (t, J=8.2 Hz, 2H), 5.03 (s, 2H), 4.19-3.99 (m, 4H), 3.47-3.27 (m, 4H); LCMS(ESI-MS): [M+H]+=496.2.

### Example 53. N-(ethylsulfonyl)-2-(2-(4-(4-hydroxyphenyl)piperazin-1-yl)-6-(1H-indazol-1-yl)-9H-purin-9-yl)acetamide

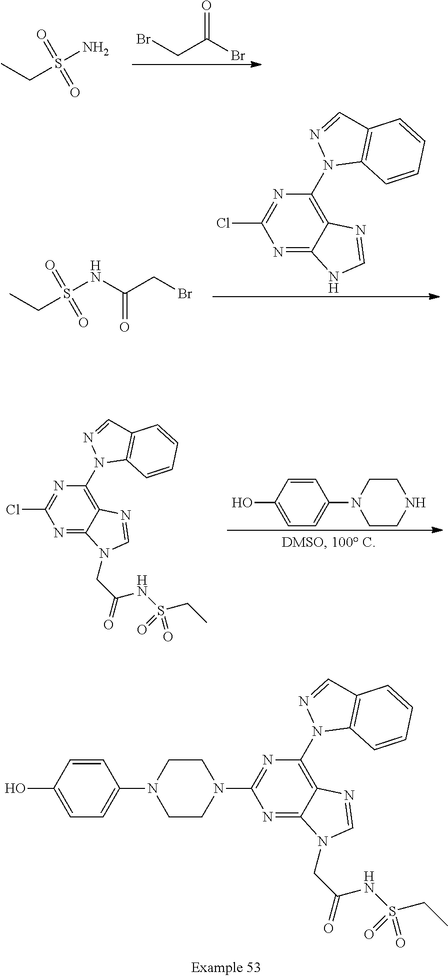

### Step 1:

To a solution of ethanesulfonamide (1.0 g, 9.17 mmol, 1.0 eq) in toluene (20 mL) at 0° C. was added bromoacetyl bromide (5.56 g, 27.52 mmol, 3 eq) dropwise. The mixture was stirred at 110° C. overnight under a nitrogen atmosphere before it was allowed to cool and diluted with water (30 mL). The mixture was extracted with EtOAc (3×30 mL). The combined organic layer was washed with brine, dried over Na₂SO₄ and concentrated. The crude residue was then purified by flash column chromatography on silica gel (petroleum ether:EtOAc=40:1) to give 2-bromo-N-(ethylsulfonyl)acetamide (0.53 g, 25% yield) as a white solid.

### Step 2:

To a solution of 2-bromo-N-(ethylsulfonyl)acetamide (0.28 g, 1.22 mmol, 3.0 eq) in DMSO (3 mL) at 25° C. was added 2-chloro-6-(1H-indazol-1-yl)-9H-purine (0.11 g, 0.41 mmol, 1.0 eq) and K₂CO₃ (1.68 g, 1.22 mmol, 3.0 eq). The mixture was stirred overnight at 100° C. under a N2 atmosphere before it was allowed to cool and diluted with water (30 mL). The mixture was adjusted to pH 4 with diluted aq. HCl solution and extracted with EtOAc (3×30 mL). The combined organic layer was washed with brine, dried over Na₂SO₄ and concentrated. The crude residue was purified by flash column chromatography on silica gel (DCM:MeOH=50:1) to give 2-(2-chloro-6-(1H-indazol-1-yl)-9H-purin-9-yl)-N-(ethylsulfonyl)acetamide (0.13 g, 24% yield) as a white solid.

### Step 3:

A mixture of 2-(2-chloro-6-(1H-indazol-1-yl)-9H-purin-9-yl)-N-(ethylsulfonyl)-acetamide (0.13 g, 0.30 mmol, 1 eq) and 1-(4-hydroxyphenyl) piperazine (0.16 g, 0.90 mmol, 3 eq) in DMSO (3 mL) was stirred overnight at 100° C. under a N₂ atmosphere. The reaction mixture allowed to cool and diluted with water (30 mL). The resulting solid was collected by filtration and purified by Prep-HPLC to give N-(ethylsulfonyl)-2-(2-(4-(4-hydroxyphenyl)-piperazin-1-yl)-6-(1H-indazol-1-yl)-9H-purin-9-yl)acetamide (42 mg, 24% yield) as a solid.

¹H NMR (400 MHz, DMSO-d6): δ 8.65 (s, 1H), 8.60 (d, J=8.4 Hz, 1H), 8.44 (s, 1H), 7.98 (d, J=7.8 Hz, 1H), 7.66 (t, J=7.5 Hz, 1H), 7.43 (t, J=7.5 Hz, 1H), 7.16 (s, 2H), 6.78 (d, J=8.7 Hz, 2H), 5.13 (s, 2H), 4.24-3.93 (m, 4H), 3.49-3.25 (m, 6H), 1.28 (t, J=7.3 Hz, 3H); LC/MS(ESI-MS): [M+H]+=562.1.

### Example 54. 2-(2-(4-(4-hydroxyphenyl)piperazin-1-yl)-6-(1H-indazol-1-yl)-9H-purin-9-yl)-N-(isopropylsulfonyl)acetamide

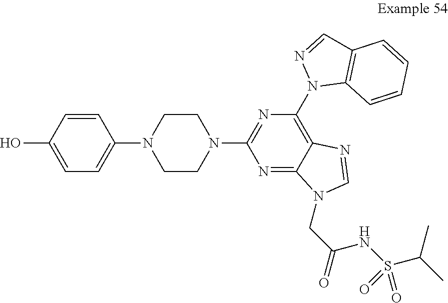 [SMILES: CC(C)S(=O)(=O)NC(=O)Cn1cnc2c(-n3ncc4ccccc43)nc(N3CCN(c4ccc(O)cc4)CC3)nc21]

The proceeding example was prepared according to Example 53 substituting ethanesulfonamide with propane-2-sulfonamide. The desired product 2-(2-(4-(4-hydroxy-phenyl)piperazin-1-yl)-6-(1H-indazol-1-yl)-9H-purin-9-yl)-N-(isopropylsulfonyl)-acetamide was isolated as a white solid.

¹H NMR (400 MHz, DMSO-d6): δ 8.64 (s, 1H), 8.60 (d, J=8.5 Hz, 1H), 8.42 (s, 1H), 7.97 (d, J=7.9 Hz, 1H), 7.66 (t, J=7.3 Hz, 1H), 7.43 (t, J=7.4 Hz, 1H), 7.23-7.05 (m, 2H), 6.77 (d, J=8.7 Hz, 2H), 5.13 (s, 2H), 4.24-3.93 (m, 4H), 3.60 (dt, J=13.7, 6.9 Hz, 1H), 3.43-3.26 (m, 4H), 1.33 (d, J=6.8 Hz, 6H); LC/MS(ESI-MS): [M+H]+=576.2.

### Example 55. N-(cyclopropylsulfonyl)-2-(2-(4-(4-hydroxyphenyl)piperazin-1-yl)-6-(1H-indazol-1-yl)-9H-purin-9-yl)acetamide

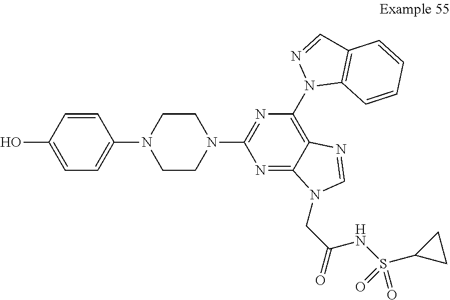 [SMILES: O=C(Cn1cnc2c(-n3ncc4ccccc43)nc(N3CCN(c4ccc(O)cc4)CC3)nc21)NS(=O)(=O)C1CC1]

The proceeding example was prepared according to Example 53 substituting ethanesulfonamide with cyclopropanesulfonamide. The desired product N-(cyclopropyl-sulfonyl)-2-(2-(4-(4-hydroxyphenyl)piperazin-1-yl)-6-(1H-indazol-1-yl)-9H-purin-9-yl)acetamide was isolated as an off-white solid.

¹H NMR (400 MHz, DMSO-d6): δ 8.65 (s, 1H), 8.60 (d, J=8.5 Hz, 1H), 8.45 (s, 11H), 7.97 (d, J=7.9 Hz, 1H), 7.66 (t, J=7.7 Hz, 1H), 7.43 (t, J=7.5 Hz, 1H), 7.25-7.05 (m, 2H), 6.77 (d, J=8.7 Hz, 2H), 5.11 (s, 2H), 4.27-3.91 (m, 4H), 3.47-3.27 (m, 4H), 3.05-2.93 (m, 1H), 1.18-1.03 (m, 4H); LC/MS(ESI-MS): [M+H]+=574.2.

### Example 56. 2-(2-(4-(4-hydroxyphenyl)piperazin-1-yl)-6-(1H-indazol-1-yl)-9H-purin-9-yl)-N-(propylsulfonyl)acetamide

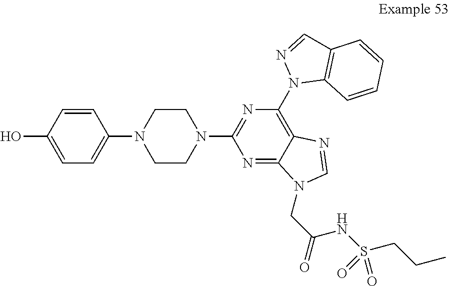 [SMILES: CCCS(=O)(=O)NC(=O)Cn1cnc2c(-n3ncc4ccccc43)nc(N3CCN(c4ccc(O)cc4)CC3)nc21]

The proceeding example was prepared according to Example 53 substituting ethanesulfonamide with propane-1-sulfonamide. The desired product 2-(2-(4-(4-hydroxy-phenyl)piperazin-1-yl)-6-(1H-indazol-1-yl)-9H-purin-9-yl)-N-(propylsulfonyl)acetamide was isolated as a white solid.

¹H NMR (400 MHz, DMSO) δ 8.65 (s, 1H), 8.60 (d, J=8.5 Hz, 1H), 8.43 (s, 1H), 7.98 (d, J=8.0 Hz, 1H), 7.66 (t, J=7.7 Hz, 1H), 7.43 (t, J=7.5 Hz, 1H), 7.26-7.06 (m, 2H), 6.77 (d, J=8.6 Hz, 2H), 5.11 (s, 2H), 4.25-3.94 (m, 4H), 3.50-3.23 (m, 6H), 1.75 (dq, J=14.9, 7.4 Hz, 2H), 1.00 (t, J=7.4 Hz, 3H); LC/MS(ESI-MS): [M+H]+=576.2.

### Example 57. 2-(2-(4-(4-hydroxyphenyl)piperazin-1-yl)-6-(1H-indazol-1-yl)-9H-purin-9-yl)-N-(methylsulfonyl)acetamide

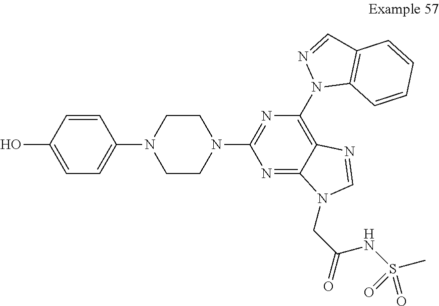 [SMILES: CS(=O)(=O)NC(=O)Cn1cnc2c(-n3ncc4ccccc43)nc(N3CCN(c4ccc(O)cc4)CC3)nc21]

The proceeding example was prepared according to Example 53 substituting ethanesulfonamide with methanesulfonamide. The desired product 2-(2-(4-(4-hydroxy-phenyl)piperazin-1-yl)-6-(1H-indazol-1-yl)-9H-purin-9-yl)-N-(methylsulfonyl)acetamide was isolated as a white solid.

¹H NMR (400 MHz, DMSO-d6): δ 8.63 (s, 1H), 8.60 (d, J=8 Hz, 1H), 8.38 (s, 1H), 7.96 (d J=8 Hz, 1H), 7.65 (t, J=8 Hz, 1H), 7.42 (t, J=8 Hz, 1H), 7.09 (bs, 2H), 6.75 (d, J=9 Hz, 2H), 5.08 (s, 3H), 4.07 (bs, 4H), 3.30 (bs, 7H); LC/MS(ESI-MS): [M+H]+=548.2.

### Example 58. 1-(9-(2-hydroxyethyl)-2-(4-(4-hydroxyphenyl)piperazin-1-yl)-9H-purin-6-yl)-1H-pyrrolo[2,3-b]pyridin-2(3H)-one

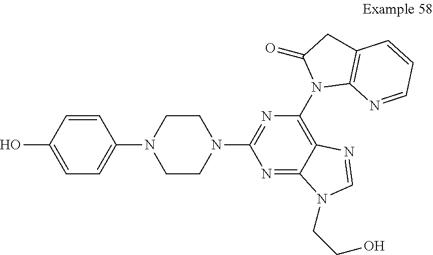 [SMILES: O=C1Cc2cccnc2N1c1nc(N2CCN(c3ccc(O)cc3)CC2)nc2c1ncn2CCO]

The proceeding example was prepared according to Example 2 substituting indolin-2-one with 1H-pyrrolo[2,3-b]pyridin-2(3H)-one. The desired product 1-(9-(2-hydroxyethyl)-2-(4-(4-hydroxyphenyl)piperazin-1-yl)-9H-purin-6-yl)-1H-pyrrolo[2,3-b]pyridin-2(3H)-one was isolated as an off-white solid.

¹HNMR (300 MHz, DMSO-d6): δ 15.12 (br, 1H), 11.13 (br, 1H), 9.28 (s, 1H), 8.90 (s, 1H), 8.13 (s, 1H), 7.88 (s, 1H), 7.09-6.83 (m, 3H), 6.76-6.61 (m, 2H), 5.02 (s, 1H), 4.19-4.07 (m, 2H), 3.92-3.69 (m, 6H), 3.14-3.01 (m, 4H); LCMS(ESI-MS): [M+H]+=473.0.

### Example 59. 4-(4-(6-methyl-9-(pyridin-3-yl)-9H-purin-2-yl)piperazin-1-yl)phenol

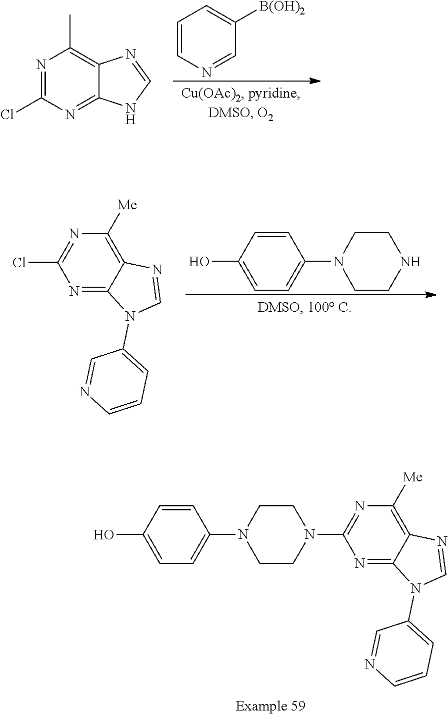

The proceeding example was prepared according to Example 37 substituting (2-chloro-3-methoxyphenyl)boronic acid with pyridin-3-ylboronic acid. The desired product 4-(4-(6-methyl-9-(pyridin-3-yl)-9H-purin-2-yl)piperazin-1-yl)phenol was isolated as a white solid.

¹H NMR (400 MHz, DMSO-d6): δ 9.18 (d, J=2 Hz, 1H), 8.85 (s, 1H), 8.64 (s, 1H), 8.62 (s, 1H), 8.37 (d, J=9 Hz, 1H), 7.66 (dd, J=5, 8 Hz, 1H), 6.84 (d, J=9 Hz, 2H), 6.66 (d, J=9 Hz, 2H), 3.89 (bs, 4H), 3.05-3.03 (m, 4H), 2.62 (s, 3H); LC/MS(ESI-MS). [M+H]+=388.1.

### Example 60. 3-(2-(4-(4-hydroxyphenyl)piperazin-1-yl)-6-methyl-9H-purin-9-yl)pyridin-2-ol

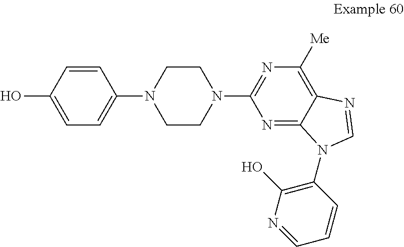 [SMILES: Cc1nc(N2CCN(c3ccc(O)cc3)CC2)nc2c1ncn2-c1cccnc1O]

The proceeding example was prepared according to Example 37 substituting (2-chloro-3-methoxyphenyl)boronic acid with (2-hydroxypyridin-3-yl)boronic acid. The desired product 3-(2-(4-(4-hydroxyphenyl)piperazin-1-yl)-6-methyl-9H-purin-9-yl)pyridin-2-ol was isolated as an off-white solid.

¹H NMR (400 MHz, DMSO-d6): δ 12.36 (s, 1H), 9.51 (s, 1H), 8.44 (d, J=2 Hz, 1H), 7.54 (d, J=3 Hz, 1H), 7.21 (b, 2H), 6.79 (d, J=8 Hz, 2H), 6.43 (t, J=7 Hz, 1H), 4.01-3.92 (m, 4H), 3.34 (bs, 4H), 2.62 (s, 3H); LC/MS(ESI-MS): [M+H]+=404.2.

### Example 61. 4-(4-(9-(2-chloro-3-methoxyphenyl)-6-methyl-9H-purin-2-yl)piperazin-1-yl)phenol

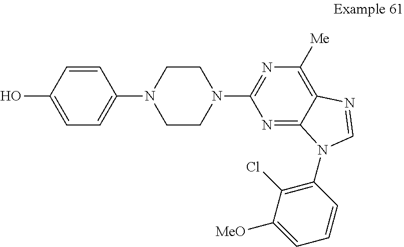 [SMILES: COc1cccc(-n2cnc3c(C)nc(N4CCN(c5ccc(O)cc5)CC4)nc32)c1Cl]

The proceeding example was prepared according to Example 37 using (2-chloro-3-methoxyphenyl)boronic acid. The desired product 4-(4-(9-(2-chloro-3-methoxyphenyl)-6-methyl-9H-purin-2-yl)piperazin-1-yl)phenol was isolated as an off-white solid.

¹H NMR (400 MHz, DMSO-d6): δ 8.39 (s, 1H), 7.27-7.22 (m, 1H), 7.06-7.01 (m, 2H), 6.93-6.87 (m, 4H), 6.50 (d, J=8.4 Hz, 1H), 3.91-3.78 (m, 7H), 3.22-3.15 (m, 4H), 2.59 (s, 3H); LC/MS(ESI-MS): [M+H]+=451.1.

### Example 62. 4-(4-(9-(2-methoxypyridin-3-yl)-6-methyl-9H-purin-2-yl)piperazin-1-yl)phenol

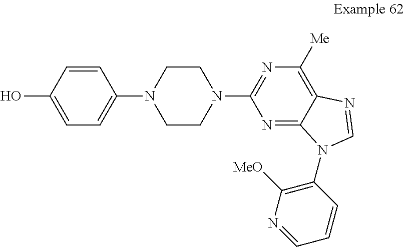 [SMILES: COc1ncccc1-n1cnc2c(C)nc(N3CCN(c4ccc(O)cc4)CC3)nc21]

The proceeding example was prepared according to Example 37 substituting (2-chloro-3-methoxyphenyl)boronic acid with (2-methoxypyridin-3-yl)boronic acid. The desired product 4-(4-(9-(2-methoxypyridin-3-yl)-6-methyl-9H-purin-2-yl)piperazin-1-yl)phenol was isolated as an off-white solid.

¹H NMR (300 MHz, DMSO-d6): δ 8.84 (s, 1H), 8.32-8.27 (m, 2H), 8.07-8.04 (m, 1H), 7.27-7.22 (m, 1H), 6.81 (d, J=8.7 Hz, 2H), 6.65 (d, J=9.0 Hz, 2H), 3.91 (s, 3H), 3.83-3.77 (m, 4H), 3.03-2.95 (m, 4H), 2.61 (s, 3H); LC/MS(ESI-MS): [M+H]+=418.2.

### Example 63. 4-(4-(9-(5-chloropyridazin-3-yl)-6-methyl-9H-purin-2-yl)piperazin-1-yl)phenol

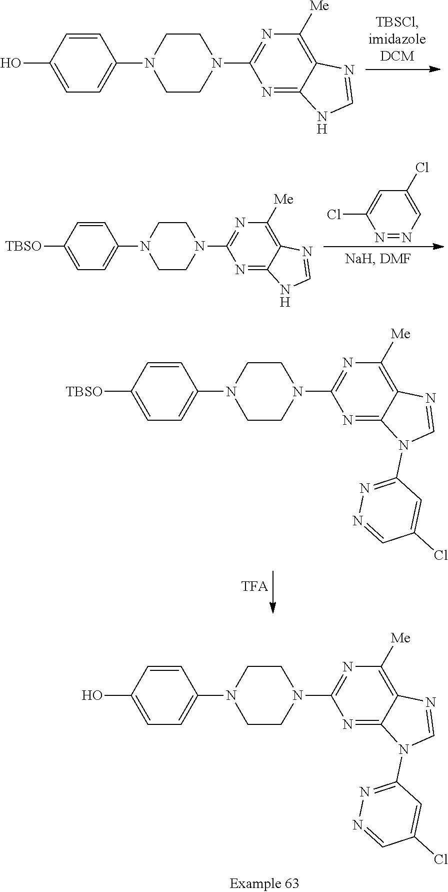 [SMILES: Cc1nc(N2CCN(c3ccc(O)cc3)CC2)nc2[nH]cnc12 ; Cc1nc(N2CCN(c3ccc(O[Si](C)(C)C(C)(C)C)cc3)CC2)nc2[nH]cnc12 ; Clc1cnnc(Cl)c1 ; Cc1nc(N2CCN(c3ccc(O[Si](C)(C)C(C)(C)C)cc3)CC2)nc2c1ncn2-c1cc(Cl)cnn1 ; Cc1nc(N2CCN(c3ccc(O)cc3)CC2)nc2c1ncn2-c1cc(Cl)cnn1]

### Step 1:

To a mixture of 4-(4-(6-methyl-9H-purin-2-yl)piperazin-1-yl)phenol (1.00 g, 3.22 mmol, 1 eq) in DCM (15 mL) at 25° C. was added imidazole (548 mg, 8.06 mmol, 2.5 eq) and TBSCl (582 mg, 3.87 mmol, 1.2 eq). The mixture was stirred for 6 h before it was diluted with DCM (15 mL) and water (5 mL). The organic layer was washed with brine, dried (Na₂SO₄) and concentrated to provide crude 2-(4-(4-((tert-butyldimethylsilyl)oxy)phenyl)-piperazin-1-yl)-6-methyl-9H-purine (1.31 g) as a solid.

### Step 2:

To a solution of 3,5-dichloropyridazine (0.50 g 3.36 mmol, 1 eq) in DMF (7 mL) at 0° C. was added NaH (134 mg, 3.36 mmol, 60% in mineral oil, 1 eq). The resulting mixture was stirred for 10 min and followed addition of crude 2-(4-(4-((tert-butyldimethylsilyl)oxy)-phenyl)piperazin-1-yl)-6-methyl-9H-purine (1.28 g, 3.02 mmol, 0.9 eq) in DMF (7 mL). The reaction mixture was stirred from 0-25° C. over a period of 5 h before it was quenched with a saturated aq. NH₄Cl solution (25 mL) and diluted with EtOAc (70 mL). The organic layer was washed with water, brine, dried (Na₂SO₄) and concentrated. The crude was purified by flash column chromatography on silica gel (0-10% MeOH in DCM) to provide 2-(4-(4-((tert-butyldimethylsilyl)oxy)phenyl)piperazin-1-yl)-9-(5-chloropyridazin-3-yl)-6-methyl-9H-purine (720 mg) as an off-white solid.

### Step 3:

To a solution of 2-(4-(4-((tert-butyldimethylsilyl)oxy)phenyl)piperazin-1-yl)-9-(5-chloropyridazin-3-yl)-6-methyl-9H-purine (100 mg, 0.19 mmol, 1 eq) in THF (5 mL) was added TFA (5 mL) at 0° C. The reaction was then stirred at 25° C. for 2 h before the reaction was concentrated under reduced pressure. The crude was purified by preparative HPLC to provide 4-(4-(9-(5-chloropyridazin-3-yl)-6-methyl-9H-purin-2-yl)piperazin-1-yl)phenol (37 mg) as an-off white solid.

¹H NMR (400 MHz, DMSO-d6): δ 9.10 (s, 1H), 8.81-8.73 (m, 1H), 7.21-7.11 (m, 5H), 3.93 (s, 4H), 3.29 (s, 4H), 2.62 (s, 3H); LC/MS(ESI-MS): [M+H]+=424.1.

### Example 64. 4-(4-(6-(2-(4-(4-hydroxyphenyl)piperazin-1-yl)-6-methyl-9H-purin-9-yl)pyridazin-4-yl)piperazin-1-yl)phenol

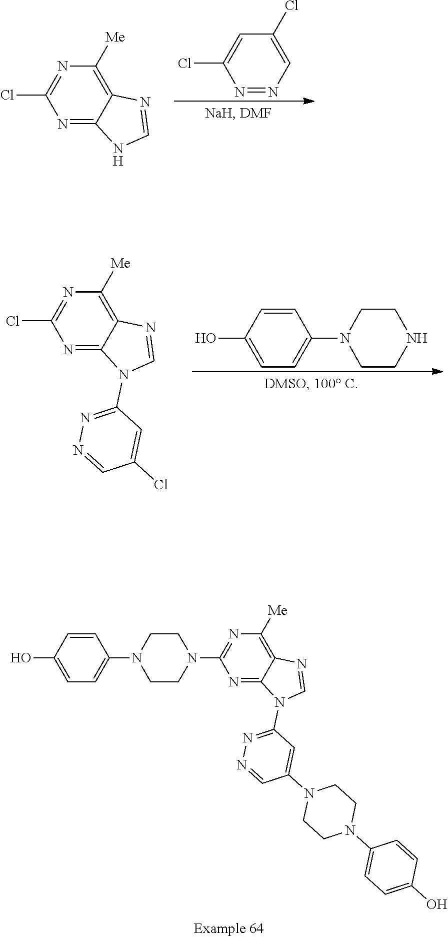 [SMILES: Cc1nc(Cl)nc2[nH]cnc12 ; Clc1cnnc(Cl)c1 ; Cc1nc(Cl)nc2c1ncn2-c1cc(Cl)cnn1 ; Oc1ccc(N2CCNCC2)cc1 ; Cc1nc(N2CCN(c3ccc(O)cc3)CC2)nc2c1ncn2-c1cc(N2CCN(c3ccc(O)cc3)CC2)cnn1]

### Step 1:

To a solution of 3,5-dichloropyridazine (100 mg 0.67 mmol, 1 eq) in DMF (3 mL) at 0° C. was added NaH (27 mg, 0.67 mmol, 60% in mineral oil, 1 eq). The resulting mixture was stirred for 10 min and followed addition of 2-chloro-6-methyl-9H-purine (103 mg, 0.60 mmol, 0.9 eq) in DMF (3 mL). The reaction mixture was stirred from 0-25° C. over a period of 5 h before it was quenched with a saturated aq. NH₄Cl solution (15 mL) and diluted with EtOAc (50 mL). The organic layer was washed with water, brine, dried (Na₂SO₄) and concentrated. The crude was purified by flash column chromatography on silica gel (0-5% MeOH in DCM) to provide 2-chloro-9-(5-chloropyridazin-3-yl)-6-methyl-9H-purine (64 mg) as an off-white solid.

### Step 2:

A mixture of 2-chloro-9-(5-chloropyridazin-3-yl)-6-methyl-9H-purine (64 mg, 0.23 mmol, 1.0 eq) and 4-(piperazin-1-yl)phenol (243 mg, 1.37 mmol, 6 eq) in DMSO (8 mL) was stirred at 100° C. overnight under nitrogen atmosphere. The reaction mixture was allowed to cool and diluted with water (15 mL). The resulting solid precipitate was collected by filtration, rinsed with water (1 mL) and dried. This crude product was further purified by Prep-HPLC to give 4-(4-(6-(2-(4-(4-hydroxyphenyl)piperazin-1-yl)-6-methyl-9H-purin-9-yl)pyridazin-4-yl)piperazin-1-yl)phenol (58 mg) as an off-white solid.

¹H NMR (400 MHz, DMSO-d6): δ 9.42 (s, 1H), 8.90-8.86 (m, 3H), 7.93 (s, 1H), 6.88-6.85 (m, 4H), 6.69-6.67 (m, 4H), 3.93 (bs, 4H), 3.84 (bs, 4H), 3.13 (bs, 4H) 3.07 (bs, 4H), 2.61 (s, 3H); LC/MS(ESI-MS): [M+H]+=566.3.

### Example 65. 4-(4-(9-(3-chlorophenyl)-6-methyl-9H-purin-2-yl)piperazin-1-yl)phenol

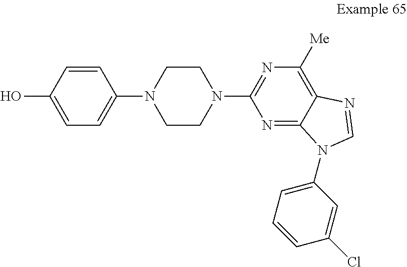 [SMILES: Cc1nc(N2CCN(c3ccc(O)cc3)CC2)nc2c1ncn2-c1cccc(Cl)c1]

The proceeding example was prepared according to Example 37 substituting (2-chloro-3-methoxyphenyl)boronic acid with (3-chlorophenyl)boronic acid. The desired product 4-(4-(9-(3-chiorophenyl)-6-methyl-9H-purin-2-yl)piperazin-1-yl)phenol was isolated as a white solid.

¹H NMR (400 MHz, DMSO-d6): δ 8.67 (s, 1H), 8.09 (s, 1H), 7.99 (d, J=8 Hz, 1H), 7.63 (t, J=8 Hz, 1H), 7.51 (d, J=8 Hz, 1H), 7.28 (bs, 2H), 6.81 (bs, 2H), 4.06 (bs, 4H), 3.43 (bs, 4H), 2.64 (s, 3H); LC/MS(ESI-MS): [M+H]+=422.1.

### Example 66. 4-(4-(6-amino-9-phenyl-9H-purin-2-yl)piperazin-1-yl)phenol

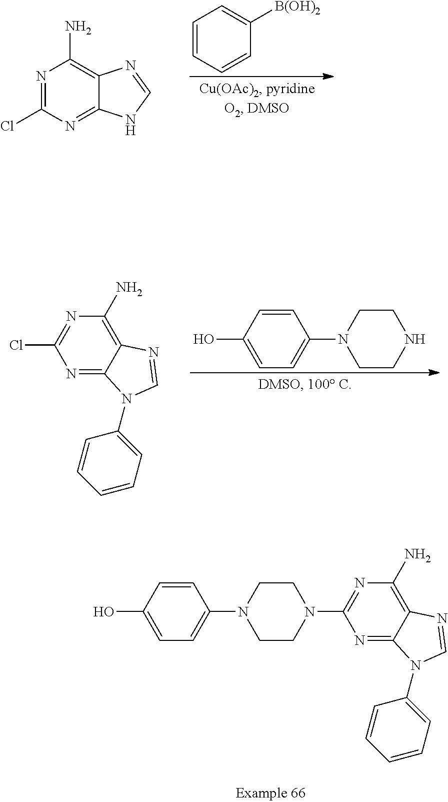 [SMILES: Nc1nc(Cl)nc2[nH]cnc12 ; OB(O)c1ccccc1 ; Nc1nc(Cl)nc2c1ncn2-c1ccccc1 ; Oc1ccc(N2CCNCC2)cc1 ; Nc1nc(N2CCN(c3ccc(O)cc3)CC2)nc2c1ncn2-c1ccccc1]

### Step 1:

To a solution of 2-chloro-9H-purin-6-amine (0.255 g, 1.5 mmol, 1.0 eq) in DMSO (10 mL) at 25° C. under an oxygen atmosphere (1 atm balloon) was added (2-chloro-3-methoxyphenyl)boronic acid (0.55 g, 4.4 mmol, 3 eq), pyridine (0.35 g, 4.4 mmol, 3 eq), anhydrous cupric acetate (0.58 g, 2.9 mmol, 2 eq). The reaction mixture was then stirred for 3 days before it was diluted with water (20 mL) and NH₄OH (1 mL). The resulting solid was collected by filtration to give crude 2-chloro-9-phenyl-9H-purin-6-amine (0.20 g) as a solid.

### Step 2:

A mixture of crude 2-chloro-9-phenyl-9H-purin-6-amine (0.2 g, 0.67 mmol, 1.0 eq) and 1-(4-hydroxyphenyl) piperazine (0.59 g, 3.35 mmol, 5.0 eq) in DMSO (5 mL) under a nitrogen atmosphere was stirred overnight at 100° C. The reaction was allowed to cool and diluted with water (20 mL). The resulting solid was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(6-amino-9-phenyl-9H-purin-2-yl)piperazin-1-yl)phenol (70 mg) as an off-white solid.

¹H NMR (400 MHz, DMSO-d6): δ 8.32 (s, 1H), 7.90 (d, J=8 Hz, 2H), 7.57 (t, J=8 Hz, 2H), 7.40 (t, J=8 Hz, 1H), 7.19 (bs, 3H), 6.79 (d, J=8 Hz, 2H), 4.30 (bs, 4H), 3.33 (bs, 4H); LC/MS(ESI-MS): [M+H]+=388.5.

### Example 67. 4-(4-(6-amino-9-(4-methylpyridin-3-yl)-9H-purin-2-yl)piperazin-1-yl)phenol

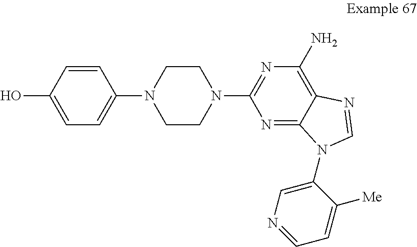 [SMILES: Cc1ccncc1-n1cnc2c(N)nc(N3CCN(c4ccc(O)cc4)CC3)nc21]

The proceeding example was prepared according to Example 66 substituting phenyl boronic acid with (4-methylpyridin-3-yl)boronic acid. The desired product 4-(4-(6-amino-9-(4-methylpyridin-3-yl)-9H-purin-2-yl)piperazin-1-yl)phenol was isolated as an off-white solid.

¹H NMR (400 MHz, DMSO-d6): δ 9.11 (bs, 1H), 8.88 (d, J=7 Hz, 1H), 8.37 (s, 1H), 8.15 (d, J=7 Hz, 1H), 7.57 (d, J=11 Hz, 1H), 6.95 (d, J=12 Hz, 1H), 4.26 (bs, 4H), 3.73 (bs, 4H), 2.59 (s, 3H); LC/MS(ESI-MS): [M+H]+=403.2.

### Example 68. 4-(4-(6-amino-9-(5-methylpyridin-3-yl)-9H-purin-2-yl)piperazin-1-yl)phenol

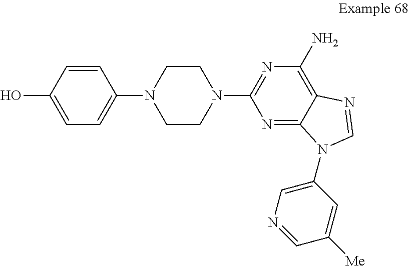 [SMILES: Cc1cncc(-n2cnc3c(N)nc(N4CCN(c5ccc(O)cc5)CC4)nc32)c1]

The proceeding example was prepared according to Example 66 substituting phenyl boronic acid with (5-methylpyridin-3-yl)boronic acid. The desired product 4-(4-(6-amino-9-(5-methylpyridin-3-yl)-9H-purin-2-yl)piperazin-1-yl)phenol was isolated as an off-white solid.

¹H NMR (400 MHz, DMSO-d6): δ 9.70 (bs, 1H), 9.05 (s, 1H), 8.48 (s, 1H), 8.40 (s, 1H), 8.22 (s, 1H), 7.30 (bs, 4H), 6.82 (d, J=8 Hz, 2H), 3.99-3.88 (m, 4H), 3.42 (bs, 4H), 2.42 (s, 3H); LC/MS(ESI-MS): [M+H]+=403.2.

### Example 69. 4-(4-(6-amino-9-(2,4-dichlorophenyl)-9H-purin-2-yl)piperazin-1-yl)phenol

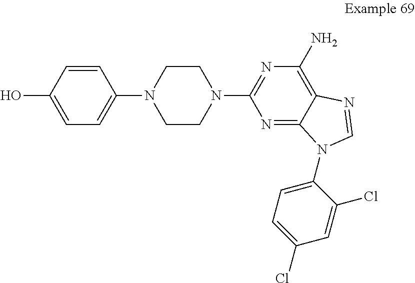 [SMILES: Nc1nc(N2CCN(c3ccc(O)cc3)CC2)nc2c1ncn2-c1ccc(Cl)cc1Cl]

The proceeding example was prepared according to Example 66 substituting phenyl boronic acid with (3,5-dichlorophenyl)boronic acid. The desired product 4-(4-(6-amino-9-(2,4-dichlorophenyl)-9H-purin-2-yl)piperazin-1-yl)phenol was isolated as an off-white solid.

¹H NMR (400 MHz, DMSO-d6): δ 8.03 (bs, 1H), 7.79 (bs, 1H), 7.59 (m, 2H), 7.45 (d, J=11 Hz, 1H), 6.93 (d, J=11 Hz, 1H), 4.15 (bs, 4H), 3.62 (bs, 4H); LC/MS(ESI-MS): [M+H]+=457.1.

### Example 70. 4-(6-amino-2-(4-(4-hydroxyphenyl)piperazin-1-yl)-9H-purin-9-yl)-3-methylbenzonitrile

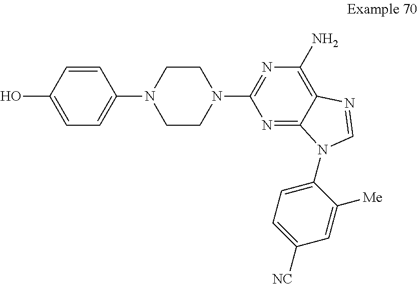 [SMILES: Cc1cc(C#N)ccc1-n1cnc2c(N)nc(N3CCN(c4ccc(O)cc4)CC3)nc21]

The proceeding example was prepared according to Example 66 substituting phenyl boronic acid with (2-chloro-4-cyano-phenyl)boronic acid. The desired product 4-(6-amino-2-(4-(4-hydroxyphenyl)piperazin-1-yl)-9H-purin-9-yl)-3-methylbenzonitrile was isolated as an off-white solid.

¹H NMR (400 MHz, DMSO-d6): δ 8.43 (s, 1H), 7.85 (bs, 2H), 7.80 (s, 1H), 7.59 (d, J=11 Hz, 1H), 7.05 (dd, J=12, 17 Hz, 4H), 6.69 (d, J=11 Hz, 1H), 3.88 (bs, 4H), 3.24 (bs, 4H), 2.30 (s, 3H); LC/MS(ESI-MS): [M+H]+=427.2.

### Example 71. 4-(4-(6-amino-9-(2-fluorophenyl)-9H-purin-2-yl)piperazin-1-yl)phenol

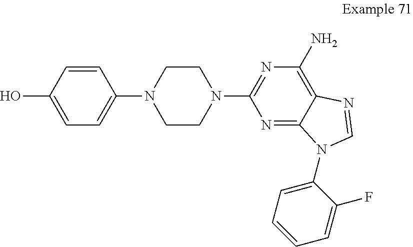 [SMILES: Nc1nc(N2CCN(c3ccc(O)cc3)CC2)nc2c1ncn2-c1ccccc1F]

The proceeding example was prepared according to Example 66 substituting phenyl boronic acid with (2-fluorophenyl)boronic acid. The desired product 4-(4-(6-amino-9-(2-fluorophenyl)-9H-purin-2-yl)piperazin-1-yl)phenol was isolated as an off-white solid.

¹H NMR (400 MHz, DMSO-d6): δ 8.09 (s, 1H), 7.74 (t, J=10 Hz, 1H), 7.56-7.40 (m, 5H), 6.93 (d, J=11 Hz, 2H), 4.19 (bs, 4H), 3.63 (bs, 4H); LC/MS(ESI-MS): [M+H]+=406.2.

### Example 72. 4-(4-(6-amino-9-(3-fluorophenyl)-9H-purin-2-yl)piperazin-1-yl)phenol

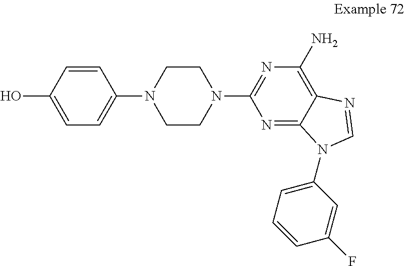 [SMILES: Nc1nc(N2CCN(c3ccc(O)cc3)CC2)nc2c1ncn2-c1cccc(F)c1]

The proceeding example was prepared according to Example 66 substituting phenyl boronic acid with (3-fluorophenyl)boronic acid. The desired product 4-(4-(6-amino-9-(3-fluorophenyl)-9H-purin-2-yl)piperazin-1-yl)phenol was isolated as a white solid.

¹H NMR (400 MHz, DMSO-d6): δ 8.28 (s, 1H), 7.77-7.54 (m, 3H), 7.45 (d, J=9 Hz, 2H), 7.20 (t, J=2 Hz, 1H), 6.93 (d, J=9 Hz, 2H), 4.23 (bs, 4H), 3.67 (bs, 4H); LC/MS(ESI-MS): [M+H]+=407.0.

### Example 73. 4-(4-(6-amino-9-(4-fluorophenyl)-9H-purin-2-yl)piperazin-1-yl)phenol

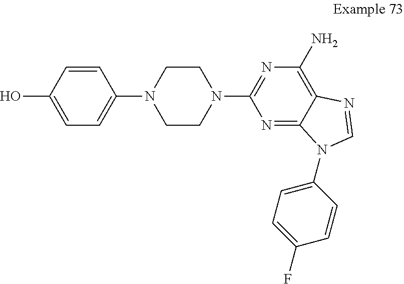 [SMILES: Nc1nc(N2CCN(c3ccc(O)cc3)CC2)nc2c1ncn2-c1ccc(F)cc1]

The proceeding example was prepared according to Example 66 substituting phenyl boronic acid with (4-fluorophenyl)boronic acid. The desired product 4-(4-(6-amino-9-(4-fluorophenyl)-9H-purin-2-yl)piperazin-1-yl)phenol was isolated as a white solid.

1H NMR (400 MHz, DMSO-d6): δ 8.25 (bs, 1H), 7.53 (bs, 2H), 7.19-7.16 (m, 2H), 7.05-6.94 (m, 6H), 3.85 (bs, 4H), 3.18 (bs, 4H); LC/MS(ESI-MS): [M+H]+=406.2.

### Example 74. 4-(6-amino-2-(4-(4-hydroxyphenyl)piperazin-1-yl)-9H-purin-9-yl)benzonitrile

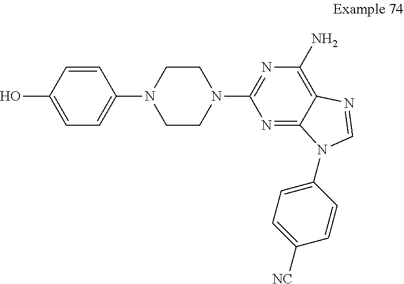 [SMILES: N#Cc1ccc(-n2cnc3c(N)nc(N4CCN(c5ccc(O)cc5)CC4)nc32)cc1]

The proceeding example was prepared according to Example 66 substituting phenyl boronic acid with (4-cyanophenyl)boronic acid. The desired product 4-(6-amino-2-(4-(4-hydroxyphenyl)piperazin-1-yl)-9H-purin-9-yl)benzonitrile was isolated as an off-white solid.

¹H NMR (400 MHz, DMSO-d6): δ 8.34 (s, 1H), 8.18 (d, J=11 Hz, 2H), 7.94 (d, J=11 Hz, 2H), 7.43 (d, J=12 Hz, 2H), 6.93 (d, J=12 Hz, 2H), 4.23 (bs, 4H), 3.63 (bs, 4H); LC/MS(ESI-MS): [M+H]+=413.2.

### Example 75. 4-(4-(6-amino-9-(4-chlorophenyl)-9H-purin-2-yl)piperazin-1-yl)phenol

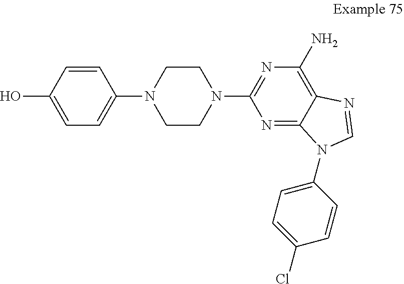 [SMILES: Nc1nc(N2CCN(c3ccc(O)cc3)CC2)nc2c1ncn2-c1ccc(Cl)cc1]

The proceeding example was prepared according to Example 66 substituting phenyl boronic acid with (4-chlorophenyl)boronic acid. The desired product 4-(4-(6-amino-9-(4-chlorophenyl)-9H-purin-2-yl)piperazin-1-yl)phenol was isolated as a white solid.

¹H NMR (400 MHz, DMSO-d6): δ 8.34 (s, 1H), 7.96 (d, J=9 Hz, 2H), 7.64 (d, J=9 Hz, 2H), 7.35-7.20 (m, 3H), 6.81 (d, J=9 Hz, 2H), 3.96 (bs, 4H), 3.37 (bs, 4H); LC/MS(ESI-MS): [M+H]+=423.0.

### Example 76. 4-(4-(6-amino-9-(o-tolyl)-9H-purin-2-yl)piperazin-1-yl)phenol

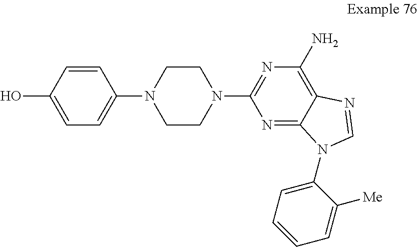 [SMILES: Cc1ccccc1-n1cnc2c(N)nc(N3CCN(c4ccc(O)cc4)CC3)nc21]

The proceeding example was prepared according to Example 66 substituting phenyl boronic acid with (2-methylphenyl)boronic acid. The desired product 4-(4-(6-amino-9-(o-tolyl)-9H-purin-2-yl)piperazin-1-yl)phenol was isolated as a white solid.

1H NMR (400 MHz, DMSO-d6): δ 8.48 (bs, 1H), 7.99 (bs, 2H), 7.28 (d, J=10 Hz, 1H), 7.16 (t, J=8 Hz, 1H), 7.05-7.01 (m, 2H), 6.86 (d, J=12 Hz, 2H), 6.75 (d, J=10 Hz, 2H), 3.87 (bs, 4H), 3.20 (bs, 4H), 2.20 (s, 3H); LC/MS(ESI-MS): [M+H]+=402.2.

### Example 77. 3-(6-amino-2-(4-(4-hydroxyphenyl)piperazin-1-yl)-9H-purin-9-yl)phenol

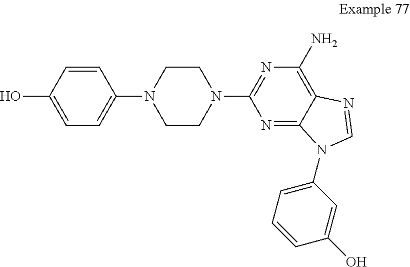 [SMILES: Nc1nc(N2CCN(c3ccc(O)cc3)CC2)nc2c1ncn2-c1cccc(O)c1]

The proceeding example was prepared according to Example 66 substituting phenyl boronic acid with (3-hydroxyphenyl)boronic acid. The desired product 3-(6-amino-2-(4-(4-hydroxyphenyl)piperazin-1-yl)-9H-purin-9-yl)phenol was isolated as an off-white solid.

¹H NMR (400 MHz, DMSO-d6): δ 9.97 (bs, 1H), 8.35 (s, 1H), 7.46-7.25 (m, 5H), 6.88-6.79 (3H), 4.20 (bs, 4H), 3.55 (bs, 4H); LC/MS(ESI-MS): [M+H]+=404.2.

### Example 78. 4-(4-(9-([1,1′-biphenyl]-3-yl)-6-amino-9H-purin-2-yl)piperazin-1-yl)phenol

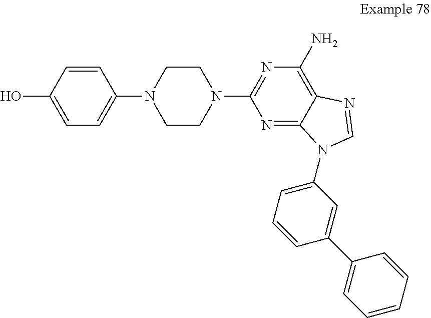 [SMILES: Nc1nc(N2CCN(c3ccc(O)cc3)CC2)nc2c1ncn2-c1cccc(-c2ccccc2)c1]

The proceeding example was prepared according to Example 66 substituting phenyl boronic acid with [1,1′-biphenyl]-3-ylboronic acid. The desired product 4-(4-(9-([1,1′-biphenyl]-3-yl)-6-amino-9H-purin-2-yl)piperazin-1-yl)phenol was isolated as a white solid.

1H NMR (400 MHz, DMSO-d6): δ 8.40 (bs, 1H), 8.11 (s, 1H), 7.80-7.64 (m, 5H), 7.50-7.39 (m, 5H), 6.92 (d, J=11 Hz, 2H), 4.23 (bs, 4H), 3.64 (bs, 4H); LC/MS(ESI-MS): [M+H]+=464.2.

### Example 79. 4-(4-(6-amino-9-(2-methoxypyridin-3-yl)-9H-purin-2-yl)piperazin-1-yl)phenol

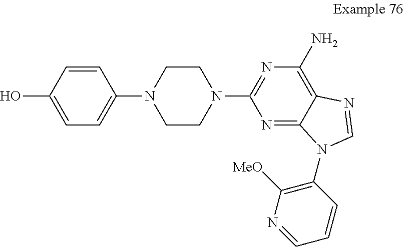 [SMILES: COc1ncccc1-n1cnc2c(N)nc(N3CCN(c4ccc(O)cc4)CC3)nc21]

The proceeding example was prepared according to Example 66 substituting phenyl boronic acid with (2-methoxypyridin-3-yl)boronic acid. The desired product 4-(4-(6-amino-9-(2-methoxypyridin-3-yl)-9H-purin-2-yl)piperazin-1-yl)phenol was isolated as an off-white solid.

¹H NMR (400 MHz, DMSO-d6): δ 8.60 (s, 1H), 8.38 (d, J=4 Hz, 1H), 7.94 (d, J=7 Hz, 1H), 7.25-7.22 (m, 1H), 6.99-6.97 (m, 2H), 6.86-6.84 (m, 1H), 6.73-6.68 (3H), 3.94-3.90 (m, 7H), 3.18 (bs, 4H); LC/MS(ESI-MS): [M+H]+=419.2.

### Example 80. 4-(4-(6-amino-9-(pyridin-4-yl)-9H-purin-2-yl)piperazin-1-yl)phenol

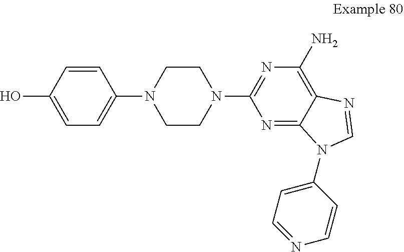 [SMILES: Nc1nc(N2CCN(c3ccc(O)cc3)CC2)nc2c1ncn2-c1ccncc1]

The proceeding example was prepared according to Example 66 substituting phenyl boronic acid with pyridin-4-ylboronic acid. The desired product 4-(4-(6-amino-9-(pyridin-4-yl)-9H-purin-2-yl)piperazin-1-yl)phenol was isolated as an off-white solid.

1H NMR (400 MHz, DMSO-d6): δ 8.90 (bs, 2H), 8.67 (s, 1H), 8.51 (bs, 2H), 7.28-7.18 (m, 4H), 6.78 (d, J=8 Hz, 2H), 4.00-3.94 (m, 4H), 3.32-3.19 (m, 4H); LC/MS(ESI-MS): [M+H]+=389.20.

### Example 81. 4-(4-(6-amino-9-(pyrimidin-5-yl)-9H-purin-2-yl)piperazin-1-yl)phenol

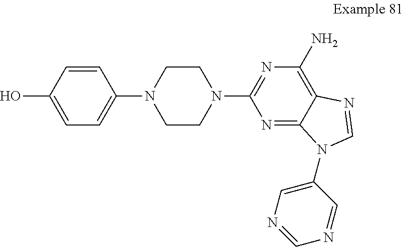 [SMILES: Nc1nc(N2CCN(c3ccc(O)cc3)CC2)nc2c1ncn2-c1cncnc1]

The proceeding example was prepared according to Example 66 substituting phenyl boronic acid with pyrimidin-5-ylboronic acid. The desired product 4-(4-(6-amino-9-(pyrimidin-5-yl)-9H-purin-2-yl)piperazin-1-yl)phenol was isolated as an off-white solid.

¹H NMR (400 MHz, DMSO-d6): δ 9.45 (s, 2H), 9.21 (s, 1H), 8.48 (s, 1H), 7.24 (bs, 3H), 6.80 (d, J=8 Hz, 2H), 3.93 (bs, 4H), 3.38 (bs, 4H); LC/MS(ESI-MS): [M+H]+=390.1.

### Example 82. 4-(4-(6-amino-9-(2-methoxypyridin-4-yl)-9H-purin-2-yl)piperazin-1-yl)phenol

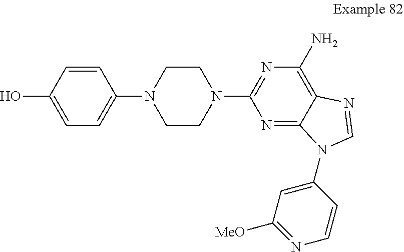 [SMILES: COc1cc(-n2cnc3c(N)nc(N4CCN(c5ccc(O)cc5)CC4)nc32)ccn1]

The proceeding example was prepared according to Example 66 substituting phenyl boronic acid with (2-methoxypyridin-4-yl)boronic acid. The desired product 4-(4-(6-amino-9-(2-methoxy-pyridin-4-yl)-9H-purin-2-yl)piperazin-1-yl)phenol was isolated as a white solid.

¹H NMR (400 MHz, DMSO-d6): δ 8.85 (s, 1H), 8.51 (s, 1H), 8.30 (d, J=6 Hx, 1H), 7.81 (d, J=7 Hz, 1H), 7.67 (s, 1H), 7.08 (bs, 2H), 6.86 (d, J=9 Hz, 2H), 6.67 (d, J=9 Hz, 2H), 3.93 (s, 3H), 3.85 (bs, 4H), 3.02 (bs, 4H); LC/MS(ESI-MS): [M+H]+=419.1.

### Example 83. 4-(4-(6-amino-9-(2-ethoxypyridin-3-yl)-9H-purin-2-yl)piperazin-1-yl)phenol

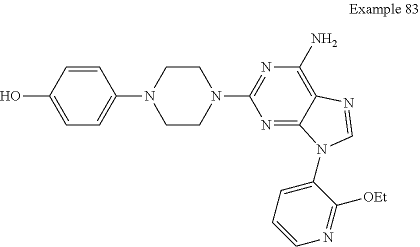 [SMILES: CCOc1ncccc1-n1cnc2c(N)nc(N3CCN(c4ccc(O)cc4)CC3)nc21]

The proceeding example was prepared according to Example 66 substituting phenyl boronic acid with (2-ethoxypyridin-3-yl)boronic acid. The desired product 4-(4-(6-amino-9-(2-ethoxy-pyridin-3-yl)-9H-purin-2-yl)piperazin-1-yl)phenol was isolated as a white solid.

¹H NMR (400 MHz, DMSO-d6): δ 8.39 (bs, 1H), 8.28-8.26 (m, 1H), 8.02 (d, J=8 Hz, 1H), 7.45 (d, J=8 Hz, 2H), 7.19 (dd, J=5, 8 Hz, 1H), 6.87 (d, J=9 Hz, 2H), 4.39 (q, J=7 Hz, 2H), 4.01 (bs, 4H), 3.55 (bs, 4H), 1.26 (t, J=7 Hz, 3H); LC/MS(ESI-MS): [M+H]+=433.2.

### Example 84. 4-(4-(6-amino-9-(2,6-dimethoxypyridin-3-yl)-9H-purin-2-yl)piperazin-1-yl)phenol

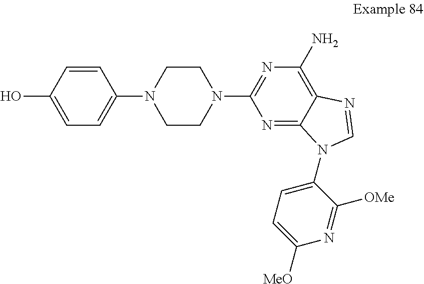 [SMILES: COc1ccc(-n2cnc3c(N)nc(N4CCN(c5ccc(O)cc5)CC4)nc32)c(OC)n1]

The proceeding example was prepared according to Example 66 substituting phenyl boronic acid with (2,6-dimethoxypyridin-3-yl)boronic acid. The desired product 4-(4-(6-amino-9-(2,6-dimethoxy-pyridin-3-yl)-9H-purin-2-yl)piperazin-1-yl)phenol was isolated as a white solid.

¹H NMR (400 MHz, DMSO-d6): δ 8.01 (s, 1H), 7.82 (d, J=8 Hz, 1H), 7.40 (d, J=9 Hz, 2H), 6.92 (d, J=9 Hz, 2H), 6.51 (d, J=8H, 1H), 4.20 (bs, 4H), 4.00 (s, 3H), 3.99 (s, 3H), 3.59 (bs, 4H); LC/MS(ESI-MS): [M+H]+=449.2.

### Example 85. 4-(4-(6-amino-9-(6-methoxypyridin-3-yl)-9H-purin-2-yl)piperazin-1-yl)phenol

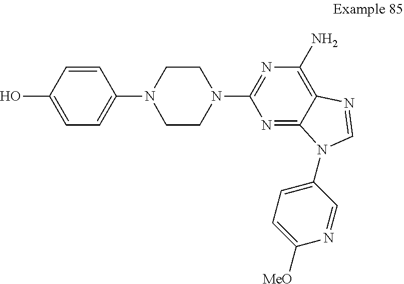 [SMILES: COc1ccc(-n2cnc3c(N)nc(N4CCN(c5ccc(O)cc5)CC4)nc32)cn1]

The proceeding example was prepared according to Example 66 substituting phenyl boronic acid with (6-methoxypyridin-3-yl)boronic acid. The desired product 4-(4-(6-amino-9-(6-methoxy-pyridin-3-yl)-9H-purin-2-yl)piperazin-1-yl)phenol was isolated as a white solid.

¹H NMR (400 MHz, DMSO-d6): δ 8.86 (s, 1H), 8.67 (d, J=3 Hz, 1H), 8.20-8.17 (m, 2H), 7.05-7.00 (m, 3H), 6.83 (d, J=12 Hz, 2H), 6.66 (d, J=12 Hz, 2H), 3.92 (s, 3H), 3.88 (bs, 4H), 2.99 (bs, 4H); LC/MS(ESI-MS): [M+H]+=419.2.

### Example 86. 5-(6-amino-2-(4-(4-hydroxyphenyl)piperazin-1-yl)-9H-purin-9-yl)picolinonitrile

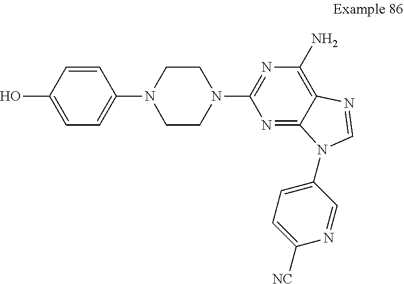 [SMILES: N#Cc1ccc(-n2cnc3c(N)nc(N4CCN(c5ccc(O)cc5)CC4)nc32)cn1]

The proceeding example was prepared according to Example 66 substituting phenyl boronic acid with (6-cyanopyridin-3-yl)boronic acid. The desired product 5-(6-amino-2-(4-(4-hydroxy-phenyl)piperazin-1-yl)-9H-purin-9-yl)picolinonitrile was isolated as a white solid.

¹H NMR (400 MHz, MeOD-d4): δ 9.45 (s, 1H), 8.62 (d, J=8 Hz, 1H), 8.43 (bs, 1H), 8.09 (d, J=9 Hz, 1H), 7.46 (d, J=9 Hz, 2H), 6.95 (d, J=9 Hz, 2H), 4.22 (bs, 4H), 3.68 (bs, 4H); LC/MS(ESI-MS): [M+H]+=414.1.

### Example 87. 4-(4-(6-amino-9-(6-chloro-2-methoxypyridin-3-yl)-9H-purin-2-yl)piperazin-1-yl)phenol

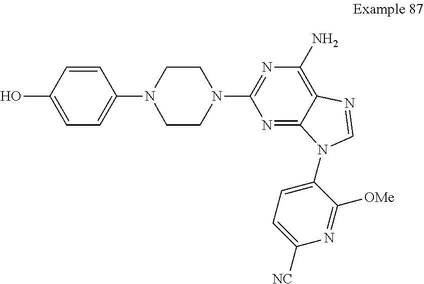 [SMILES: COc1nc(C#N)ccc1-n1cnc2c(N)nc(N3CCN(c4ccc(O)cc4)CC3)nc21]

The proceeding example was prepared according to Example 66 substituting phenyl boronic acid with (6-chloro-2-methoxypyridin-3-yl)boronic acid. The desired product 4-(4-(6-amino-9-(6-chloro-2-methoxypyridin-3-yl)-9H-purin-2-yl)piperazin-1-yl)phenol was isolated as a white solid.

¹H NMR (400 MHz, MeOD-d4): δ 8.45 (bs, 1H), 8.06 (d, J=8 Hz, 1H), 7.51 (d, J=9 Hz, 2H), 7.24 (d, J=8 Hz, 2H), 6.95 (d, J=9H, 1H), 4.02 (s, 3H), 3.69 (bs, 4H), 3.33 (bs, 4H); LC/MS(ESI-MS): [M+H]+=454.1.

### Example 88. 4-(4-(6-amino-9-(1-methyl-1H-pyrazol-4-yl)-9H-purin-2-yl)piperazin-1-yl)phenol

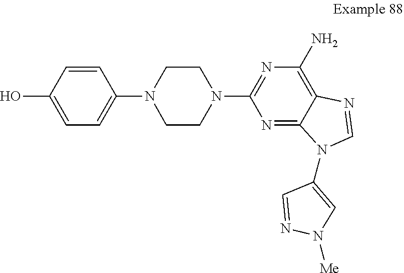 [SMILES: Cn1cc(-n2cnc3c(N)nc(N4CCN(c5ccc(O)cc5)CC4)nc32)cn1]

The proceeding example was prepared according to Example 66 substituting phenyl boronic acid with (1-methyl-1H-pyrazol-4-yl)boronic acid. The desired product 4-(4-(6-amino-9-(1-methyl-1H-pyrazol-4-yl)-9H-purin-2-yl)piperazin-1-yl)phenol was isolated as a white solid. LC/MS(ESI-MS): [M+H]+=392.1.

### Example 89. 4-(4-(6-amino-9-benzyl-9H-purin-2-yl)piperazin-1-yl)phenol

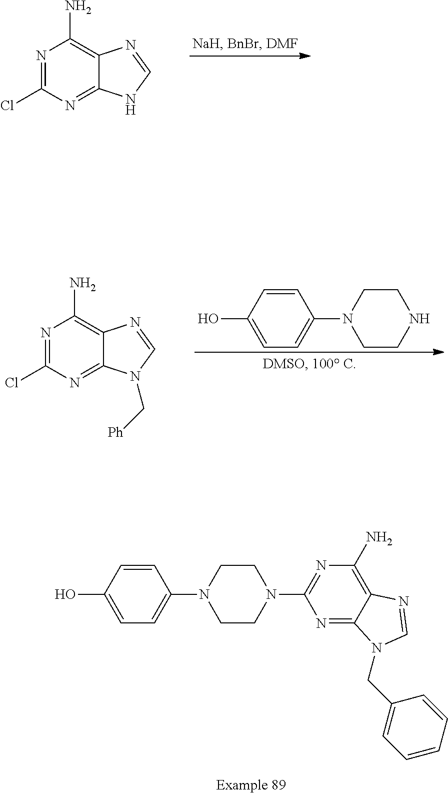 [SMILES: Nc1nc(Cl)nc2[nH]cnc12 ; Nc1nc(Cl)nc2c1ncn2Cc1ccccc1 ; Oc1ccc(N2CCNCC2)cc1 ; Nc1nc(N2CCN(c3ccc(O)cc3)CC2)nc2c1ncn2Cc1ccccc1]

### Step 1:

To a solution of 2-chloro-9H-purin-6-amine (0.2 g, 1.18 mmol, 1.0 eq) in DMF (5 mL) under nitrogen atmosphere at 0° C. was added sodium hydride (56 mg, 1.41 mol, 1.2 eq, 60% in oil) in portions. The mixture was stirred for 0.5 h and followed by addition of benzyl bromide (0.3 g, 1.76 mmol, 1.5 eq). Then the resulting mixture was stirred at 25° C. overnight before it was diluted with water (10 mL). The solid was collected by filtration and dried to give crude 9-benzyl-2-chloro-9H-purin-6-amine (0.2 g, 67% yield) as an off-white solid.

### Step 2:

To a solution of crude 9-benzyl-2-chloro-9H-purin-6-amine (0.2 g, 0.77 mmol, 1.0 eq) in DMSO (5 mL) was added 1-(4-hydroxyphenyl) piperazine (0.68 g, 3.86 mmol, 5.0 eq). The mixture was stirred overnight at 100° C. under nitrogen. The reaction mixture allowed to cool and diluted with water (15 mL). The resulting solid was collected by filtration and purified by Prep-HPLC to give 4-(4-(6-amino-9-benzyl-9H-purin-2-yl)piperazin-1-yl)phenol (51 mg, 16% yield) as a white solid.

¹H NMR (400 MHz, DMSO-d6): δ 8.85 (br, 1H), 7.87 (s, 1H), 7.34-7.22 (m, 4H), 6.88-6.67 (m, 6H), 5.22 (s, 2H), 3.83-3.76 (m, 4H), 2.97-2.91 (m, 4H); LC/MS(ESI-MS): [M+H]+=402.2.

### Example 90. 4-(4-(6-(benzylamino)-9-(pyridin-3-yl)-9H-purin-2-yl)piperazin-1-yl)phenol

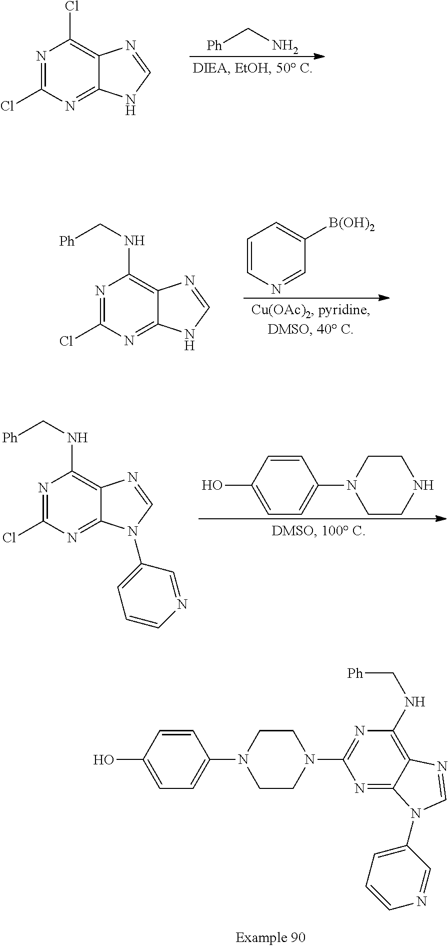

### Step 1:

To a mixture of 2,6-dichloropurine (0.5 g, 2.6 mmol, 1.0 eq) in EtOH (5 mL) was added benzylamine (0.28 g, 2.6 mmol, 1.0 eq) and DIEA (0.4 g, 4.0 mmol, 1.5 eq). The mixture was stirred at 50° C. for 3 days before it was allowed to cool. The resulting precipitate was collected by filtration and dried to give N-benzyl-2-chloro-9H-purin-6-amine (0.6 g, 88% yield) as a white solid.

### Step 2:

To a solution of N-benzyl-2-chloro-9H-purin-6-amine (0.6 g, 2.32 mmol, 1.0 eq) in DMSO (10 mL) was added pyridin-3-ylboronic acid (0.57 g, 4.63 mmol, 2.0 eq), pyridine (0.55 g, 6.95 mmol, 3.0 eq), and anhydrous cupric acetate (0.93 g, 4.63 mmol, 2.0 eq). The mixture was stirred for 3 days at 25° C. before it was diluted with water (30 mL) and NHO₃.H₂O (1 mL). The resulting solid was collected by filtration and dried to give crude N-benzyl-2-chloro-9-(pyridin-3-yl)-9H-purin-6-amine (0.11 g, 14% yield).

### Step 3:

A mixture of crude N-benzyl-2-chloro-9-(pyridin-3-yl)-9H-purin-6-amine (0.11 g, 0.33 mmol, 1.0 eq) and 1-(4-hydroxyphenyl) piperazine (0.29 g, 1.64 mmol, 5.0 eq) in DMSO (5 mL) was stirred overnight at 100° C. under a nitrogen atmosphere. The reaction mixture was allowed to cool and diluted with water (20 mL). The resulting precipitate was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 4-(4-(6-(benzylamino)-9-(pyridin-3-yl)-9H-purin-2-yl)piperazin-1-yl)phenol (27 mg, 17% yield) as a white solid.

¹H NMR (400 MHz, DMSO-d6): δ 9.17 (s, 1H), 8.61 (d, J=4.0 Hz, 1H), 8.40-8.35 (m, 3H), 7.65-7.62 (m, 1H), 7.39 (d, J=7.6 Hz, 1H), 7.32-7.19 (m, 4H), 6.79 (d, J=8.8 Hz, 1H), 4.65 (s, 2H), 4.01-3.97 (m, 4H), 3.36-3.27 (m, 4H); LC/MS(ESI-MS): [M+H]+=479.2.

### Example 91. 4-(4-(6-(phenethylamino)-9-(pyridin-3-yl)-9H-purin-2-yl)piperazin-1-yl)phenol

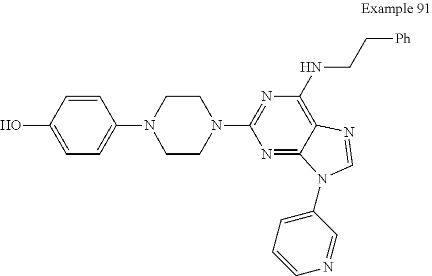 [SMILES: Oc1ccc(N2CCN(c3nc(NCCc4ccccc4)c4ncn(-c5cccnc5)c4n3)CC2)cc1]

The proceeding example was prepared according to Example 90 substituting benzylamine with phenethylamine. The desired product 4-(4-(6-(phenethylamino)-9-(pyridin-3-yl)-9H-purin-2-yl)piperazin-1-yl)phenol was isolated as a white solid.

1H NMR (400 MHz, CD3OD-d4): δ 9.26 (br, 1H), 8.67 (br, 1H), 8.46 (d, J=8.4 Hz, 1H), 8.31 (s, 1H), 7.74 (s, 1H), 7.50 (d, J=8.8 Hz, 1H), 7.30-7.26 (m, 5H), 7.22-7.19 (m, 1H), 6.97 (d, J=8.8 Hz, 2H), 4.33-4.16 (m, 4H), 3.89-3.81 (m, 2H), 3.74-3.69 (m, 4H), 3.02 (t, J=7.2 Hz, 2H); LC/MS(ESI-MS): [M+H]+=493.2.

### Example 92. 4-(4-(9-(pyridin-3-yl)-6-(pyrrolidin-1-yl)-9H-purin-2-yl)piperazin-1-yl)phenol

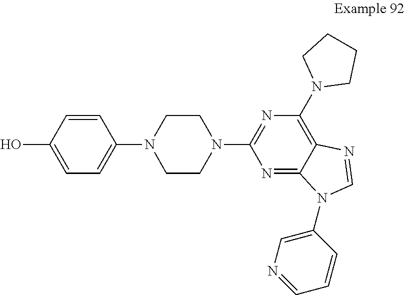 [SMILES: Oc1ccc(N2CCN(c3nc(N4CCCC4)c4ncn(-c5cccnc5)c4n3)CC2)cc1]

The proceeding example was prepared according to Example 39 substituting benzyamine with pyrrolidine. The desired product 4-(4-(9-(pyridin-3-yl)-6-(pyrrolidin-1-yl)-9H-purin-2-yl)piperazin-1-yl)phenol was isolated as a white solid.

¹H NMR (400 MHz, MeOD-d4): δ 9.21 (s, 1H), 8.64 (s, 1H), 8.46-8.43 (m, 1H), 8.26 (s, 1H), 7.74-7.70 (m, 1H), 7.52-7.48 (m, 2H), 6.98-6.95 (m, 2H), 4.34-4.01 (m, 6H), 3.85-3.70 (m, 6H), 2.19-1.95 (m, 4H); LC/MS(ESI-MS): [M+H]+=443.2.

### Example 93. (R)-4-(4-(6-(2-(hydroxymethyl)pyrrolidin-1-yl)-9-(pyridin-3-yl)-9H-purin-2-yl)piperazin-1-yl)phenol

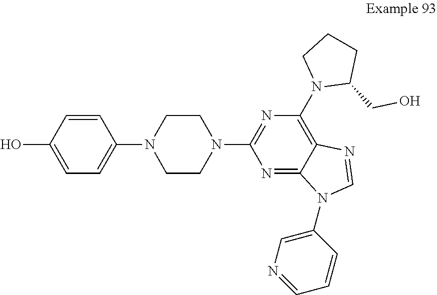 [SMILES: OC[C@H]1CCCN1c1nc(N2CCN(c3ccc(O)cc3)CC2)nc2c1ncn2-c1cccnc1]

The proceeding example was prepared according to Example 39 substituting benzylamine with (R)-pyrrolidin-2-ylmethanol. The desired (R)-4-(4-(6-(2-(hydroxymethyl)-pyrrolidin-1-yl)-9-(pyridin-3-yl)-9H-purin-2-yl)piperazin-1-yl)phenol was isolated as a white solid.

¹H NMR (400 MHz, MeOD-d4): δ 9.18 (s, 1H), 8.61 (s, 1H), 8.38 (d, J=8.0 Hz, 1H), 8.23 (s, 1H), 7.69-7.66 (m, 1H), 7.46 (d, J=8.8 Hz, 2H), 6.93 (d, J=8.8 Hz, 1H), 4.71-4.44 (m, 1H), 4.37-3.99 (m, 4H), 3.89-3.86 (m, 2H), 3.68-3.61 (m, 4H), 2.86-2.74 (m, 2H), 2.23-1.98 (m, 4H); LC/MS(ESI-MS): [M+H]+=473.2.

### Example 94. 4-(4-(6-(3-(hydroxymethyl)pyrrolidin-1-yl)-9-(pyridin-3-yl)-9H-purin-2-yl)piperazin-1-yl)phenol

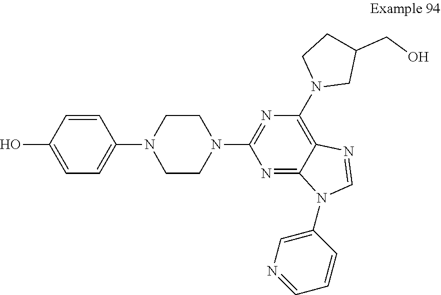 [SMILES: OCC1CCN(c2nc(N3CCN(c4ccc(O)cc4)CC3)nc3c2ncn3-c2cccnc2)C1]

The proceeding example was prepared according to Example 39 substituting benzylamine with pyrrolidin-3-ylmethanol. The desired 4-(4-(6-(3-(hydroxymethyl)-pyrrolidin-1-yl)-9-(pyridin-3-yl)-9H-purin-2-yl)piperazin-1-yl)phenol was isolated as a white solid.

¹H NMR (400 MHz, DMSO-d6): δ 8.50 (d, J=8.0 Hz, 1H), 8.26 (s, 1H), 7.80 (s, 1H), 7.49 (d, J=9 Hz, 2H), 6.94 (d, J=9 Hz, 1H), 4.25 (bs, 4H), 3.89-3.69 (m, 10H), 2.65-2.52 (m, 1H), 2.18 (m, 1H), 1.86 (m, 1H); LC/MS(ESI-MS): [M+H]+=474.0.

### Example 95. 4-(4-(6-((cyclopropylmethyl)amino)-9-(pyridin-3-yl)-9H-purin-2-yl)piperazin-1-yl)phenol

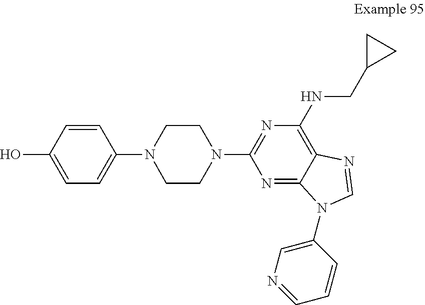 [SMILES: Oc1ccc(N2CCN(c3nc(NCC4CC4)c4ncn(-c5cccnc5)c4n3)CC2)cc1]

The proceeding example was prepared according to Example 39 substituting benzylamine with cyclopropylmethylamine. The desired 4-(4-(6-((cyclopropylmethyl)-amino)-9-(pyridin-3-yl)-9H-purin-2-yl)piperazin-1-yl)phenol was isolated as a white solid.

1H NMR (300 MHz, MeOD-d4): δ 9.18 (s, 1H), 8.59 (s, 1H), 8.41-8.36 (m, 1H), 8.21 (s, 1H), 7.71-7.62 (m, 1H), 6.94 (d, J=8.7 Hz, 2H), 6.74 (d, J=8.7 Hz, 2H), 4.03-3.95 (m, 4H), 3.50-3.41 (m, 2H), 3.09-3.01 (m, 4H), 1.31-1.22 (m, 1H), 0.57 (d, J=6.9 Hz, 2H), 0.35 (d, J=4.2 Hz, 2H); LC/MS(ESI-MS): [M+H]+=443.2.

### Example 96. 4-(4-(6-(cyclopentylamino)-9-(pyridin-3-yl)-9H-purin-2-yl)piperazin-1-yl)phenol

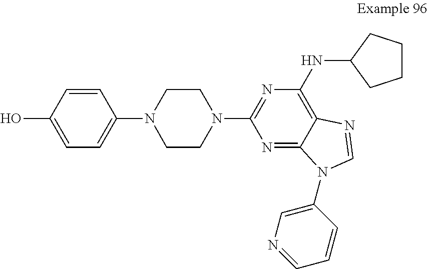 [SMILES: Oc1ccc(N2CCN(c3nc(NC4CCCC4)c4ncn(-c5cccnc5)c4n3)CC2)cc1]

The proceeding example was prepared according to Example 39 substituting benzylamine with cyclopentylamine. The desired 4-(4-(6-(cyclopentylamino)-9-(pyridin-3-yl)-9H-purin-2-yl)piperazin-1-yl)phenol was isolated as a white solid.

1H NMR (400 MHz, DMSO-d6): δ 9.20 (s, 1H), 8.62 (s, 1H), 8.41 (d, J=8 Hz, 1H), 8.29 (s, 1H), 7.71-7.67 (m, 1H), 7.48 (d, J=9 Hz, 2H), 6.94 (d, J=9 Hz, 2H), 4.54 (s, 1H), 4.24 (bs, 4H), 3.70 (bs, 4H), 3.30 (bs, 4H), 2.11 (bs, 2H), 1.82 (bs, 2H), 1.67-1.64 (m, 4H); LC/MS(ESI-MS): [M+H]+=458.0.

### Example 97. 2-(2-(4-(4-hydroxyphenyl)piperazin-1-yl)-6-(1H-indazol-1-yl)-9H-purin-9-yl)-N-(phenylsulfonyl)acetamide

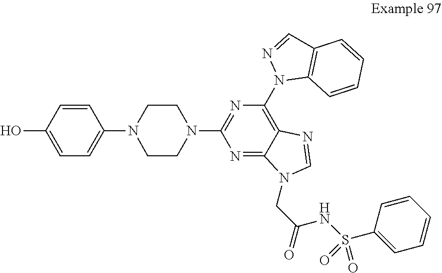 [SMILES: O=C(Cn1cnc2c(-n3ncc4ccccc43)nc(N3CCN(c4ccc(O)cc4)CC3)nc21)NS(=O)(=O)c1ccccc1]

The proceeding example was prepared according to Example 53 substituting ethanesulfonamide with benzenesulfonamide. The desired product 2-(2-(4-(4-hydroxy-phenyl)piperazin-1-yl)-6-(1H-indazol-1-yl)-9H-purin-9-yl)-N-(phenylsulfonyl)acetamide was isolated as a white solid.

¹H NMR (400 MHz, DMSO-d6): δ 9.68 (s, 1H), 8.24 (s, 1H), 8.00-7.89 (m, 2H), 7.84 (d, J=8.6 Hz, 1H), 7.77 (d, J=8.9 Hz, 1H), 7.71 (t, J=7.4 Hz, 1H), 7.64 (t, J=7.5 Hz, 2H), 7.39-7.32 (m, 1H), 7.14 (dd, J=8.4, 6.6 Hz, 1H), 7.01-6.91 (m, 2H), 6.73 (d, J=8.6 Hz, 2H), 5.04 (s, 2H), 3.97-3.84 (m, 4H), 3.15-3.05 (m, 4H); LC/MS(ESI-MS): [M+H]+=610.2.

### Example 98. N-(benzylsulfonyl)-2-(2-(4-(4-hydroxyphenyl)piperazin-1-yl)-6-(1H-indazol-1-yl)-9H-purin-9-yl)acetamide

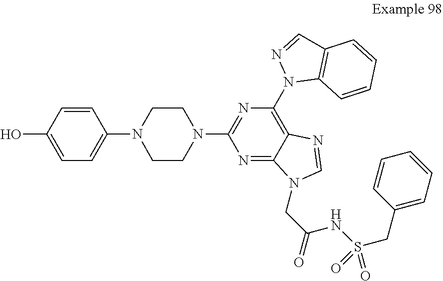 [SMILES: O=C(Cn1cnc2c(-n3ncc4ccccc43)nc(N3CCN(c4ccc(O)cc4)CC3)nc21)NS(=O)(=O)Cc1ccccc1]

The proceeding example was prepared according to Example 53 substituting ethyl sulfonamide with benzyl sulfonamide. The desired product N-(benzylsulfonyl)-2-(2-(4-(4-hydroxyphenyl)piperazin-1-yl)-6-(1H-indazol-1-yl)-9H-purin-9-yl)acetamide was isolated as a white solid.

¹H NMR (400 MHz, DMSO-d6): δ 9.75 (s, 1H), 8.38 (s, 1H), 7.87 (d, J=8.5 Hz, 1H), 7.80 (d, J=8.9 Hz, 1H), 7.47-7.31 (m, 6H), 7.19-7.13 (m, 1H), 7.12-6.99 (m, 2H), 6.75 (d, J=8.1 Hz, 2H), 5.07 (s, 2H), 4.73 (s, 3H), 4.18-4.07 (m, 4H), 3.36-3.21 (m, 4H); LC/MS(ESI-MS): [M+H]+=624.2.

### Example 99. 1-(9-(carboxymethyl)-2-(4-(4-hydroxyphenyl)piperazin-1-yl)-9H-purin-6-yl)-1H-indazole-4-carboxylic acid

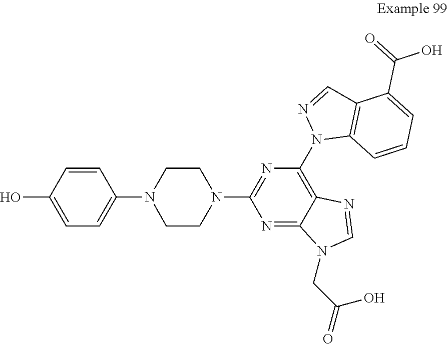 [SMILES: O=C(O)Cn1cnc2c(-n3ncc4c(C(=O)O)cccc43)nc(N3CCN(c4ccc(O)cc4)CC3)nc21]

The proceeding example was prepared according to Example 10 substituting ethyl 1H-indazole-3-carboxylate with ethyl 1H-indazole-4-carboxylate. The desired product 1-(9-(carboxymethyl)-2-(4-(4-hydroxyphenyl)piperazin-1-yl)-9H-purin-6-yl)-1H-indazole-4-carboxylic acid was isolated as a white solid.

¹H NMR (400 MHz, DMSO-d6): δ 13.45 (br, 1H), 8.88 (s, 1H), 8.85 (d, J=8.0 Hz, 1H), 8.37 (bs, 1H), 8.03 (d, J=6.7 Hz, 1H), 7.76 (t, J=8.0 Hz, 1H), 7.09 (bs, 2H), 6.76 (d, J=8.0 Hz, 2H), 5.04 (s, 2H), 4.03-3.88 (m, 4H), 3.32 (bs, 4H); LC/MS (ESI-MS): [M+H]+=515.1.

### Example 100. 1-(9-(2-(tert-butoxy)-2-oxoethyl)-2-(4-(4-hydroxyphenyl)piperazin-1-yl)-9H-purin-6-yl)-1H-benzo[d][1,2,3]triazole-4-carboxylic acid

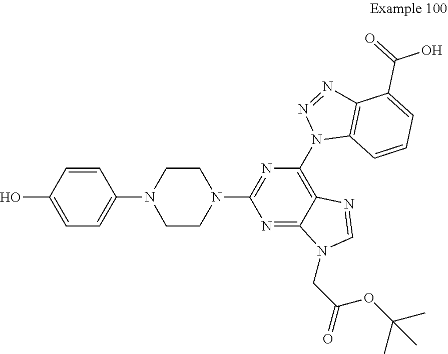 [SMILES: CC(C)(C)OC(=O)Cn1cnc2c(-n3nnc4c(C(=O)O)cccc43)nc(N3CCN(c4ccc(O)cc4)CC3)nc21]

The proceeding example was prepared according to Example 24 substituting 1H-benzo[d][1,2,3]triazole and ethyl 2-(2,6-dichloro-9H-purin-9-yl)acetate with ethyl 1H-benzo[d][1,2,3]triazole-4-carboxylate and tert-butyl 2-(2,6-dichloro-9H-purin-9-yl)acetate. The desired product 1-(9-(carboxymethyl)-2-(4-(4-hydroxyphenyl)piperazin-1-yl)-9H-purin-6-yl)-1H-indazole-4-carboxylic acid was isolated as a white solid.

¹H NMR (400 MHz, DMSO-d6): δ 8.89 (s, 1H), 8.70 (d, J=8 Hz, 1H), 8.32 (s, 1H), 8.11 (d, J=7 Hz, 1H), 7.88 (t, J=8 Hz, 1H), 6.88 (d, J=8 Hz, 2H), 6.68 (d, J=8 Hz, 2H), 5.03 (s, 2H), 4.05 (bs, 4H), 3.17 (bs, 4H), 1.46 (s, 9H); LC/MS(ESI-MS): [M+H]+=572.2.

### Example 101. methyl 4-(6-amino-2-(4-(4-hydroxyphenyl)piperazin-1-yl)-9H-purin-9-yl)benzoate

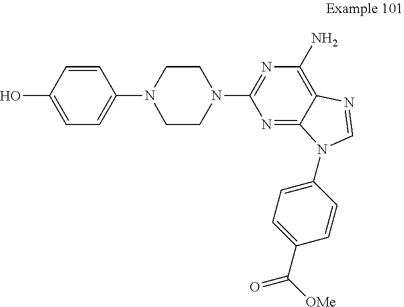 [SMILES: COC(=O)c1ccc(-n2cnc3c(N)nc(N4CCN(c5ccc(O)cc5)CC4)nc32)cc1]

The proceeding example was prepared according to Example 66 substituting phenyl boronic acid with (4-(methoxycarbonyl)phenyl)boronic acid. The desired product methyl 4-(6-amino-2-(4-(4-hydroxyphenyl)piperazin-1-yl)-9H-purin-9-yl)benzoate was isolated as a white solid.

¹H NMR (400 MHz, DMSO-d6): δ 8.45 (s, 1H), 8.20-8.12 (m, 4H), 7.22 (bs, 3H), 6.79 (d, J=8 Hz, 2H), 3.90 (bs, 7H), 3.34 (bs, 4H); LC/MS(ESI-MS): [M+H]+=446.1.

### Example 102. 4-(6-amino-2-(4-(4-hydroxyphenyl)piperazin-1-yl) 9H-purin-9-yl)benzoic acid

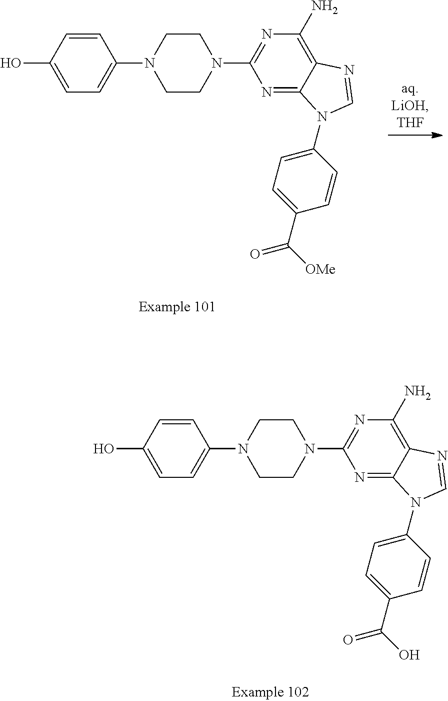 [SMILES: COC(=O)c1ccc(-n2cnc3c(N)nc(N4CCN(c5ccc(O)cc5)CC4)nc32)cc1 ; Nc1nc(N2CCN(c3ccc(O)cc3)CC2)nc2c1ncn2-c1ccc(C(=O)O)cc1]

To a solution of methyl 4-(6-amino-2-(4-(4-hydroxyphenyl)piperazin-1-yl)-9H-purin-9-yl)benzoate (100 mg, 0.22 mmol, 1 eq) in THF (2 mL) and H₂O (3 mL) at 25° C. was added LiOH monohydrate (28 mg, 0.67 mmol, 3 eq). The mixture was stirred for 4 h before the organic volatile was removed under reduced pressure. The aqueous layer was acidified with 1N aq. HCl solution to pH 6 and extracted with EtOAc (15 mL). The organic layer was washed with brine, dried over Na₂SO₄ and concentrated to provide 4-(6-amino-2-(4-(4-hydroxyphenyl)piperazin-1-yl)-9H-purin-9-yl)benzoic acid (75 mg) as a white solid.

¹H NMR (400 MHz, DMSO-d6): δ 8.43 (s, 1H), 8.15-8.09 (m, 4H), 7.23-7.10 (m, 3H), 6.79-6.77 (m, 2H), 4.05-3.99 (m, 4H), 3.38-3.21 (m, 4H); LC/MS(ESI-MS): [M+H]+=432.1.

### Example 103. 4-(6-amino-2-(4-(4-hydroxyphenyl)piperazin-1-yl)-9H-purin-9-yl)-N-methylbenzamide

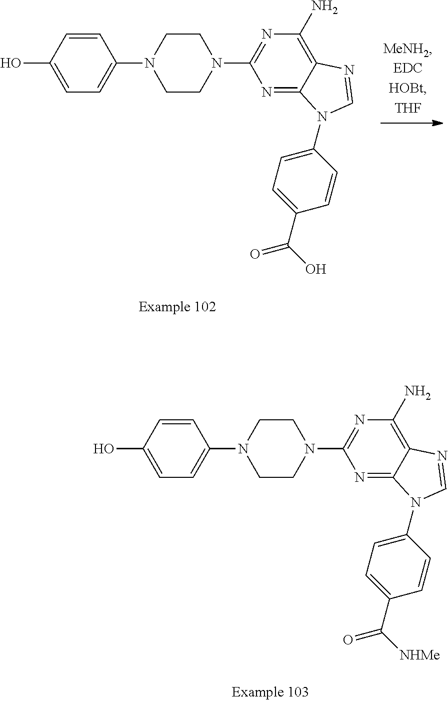 [SMILES: Nc1nc(N2CCN(c3ccc(O)cc3)CC2)nc2c1ncn2-c1ccc(C(=O)O)cc1 ; CNC(=O)c1ccc(-n2cnc3c(N)nc(N4CCN(c5ccc(O)cc5)CC4)nc32)cc1]

To a solution of 4-(6-amino-2-(4-(4-hydroxyphenyl)piperazin-1-yl)-9H-purin-9-yl)benzoic acid (50 mg, 0.16 mmol, 1 eq) in THF (2 mL) at 0° C. was added 1N MeNH2 solution in THF (0.17 mL, 0.17 mmol, 1.5 eq), EDC (44 mg, 0.23 mmol, 2 eq) and HOBt (31 mg, 0.23 mmol, 2 eq). The mixture was stirred for 3 h at 25° C. before the organic volatile was removed under reduced pressure. The aqueous layer was extracted with EtOAc (2×15 mL). The combined organic layer was washed with brine, dried over Na₂SO₄ and concentrated. The crude residue was purify by flash column chromatography on silica gel (0-5% MeOH in DCM) to provide 4-(6-amino-2-(4-(4-hydroxyphenyl)piperazin-1-yl)-9H-purin-9-yl)-N-methylbenzamide (41 mg) as a white solid.

¹H NMR (400 MHz, DMSO-d6): δ 8.54 (d, J=5 Hz, 1H), 8.43 (s, 1H), 8.07-8.01 (m, 5H), 7.35-7.21 (m, 4H), 6.84 (d, J=8 Hz, 2H), 5.00 (bs, 4H), 3.47 (bs, 4H), 2.89 (s, 3H); LC/MS(ESI-MS): [M+H]+=445.9.

### Example 104. 1-(9-(carboxymethyl)-2-(4-(4-hydroxyphenyl)piperazin-1-yl)-9H-purin-6-yl)-1H-indazole-6-carboxylic acid

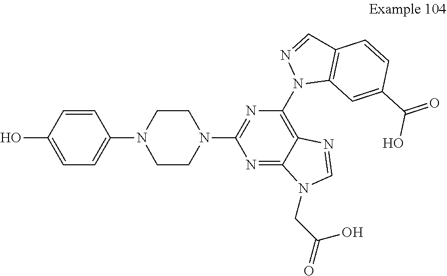 [SMILES: O=C(O)Cn1cnc2c(-n3ncc4ccc(C(=O)O)cc43)nc(N3CCN(c4ccc(O)cc4)CC3)nc21]

The proceeding example was prepared according to Example 10 substituting ethyl 1H-indazole-3-carboxylate with ethyl 1H-indazole-6-carboxylate. The desired product 1-(9-(carboxymethyl)-2-(4-(4-hydroxyphenyl)piperazin-1-yl)-9H-purin-6-yl)-1H-indazole-6-carboxylic acid was isolated as a white solid.

¹H NMR (400 MHz, DMSO-d6): δ 9.38 (s, 1H), 8.72 (s, 1H), 8.37 (d, J=22.9 Hz, 1H), 8.05 (d, J=8.3 Hz, 1H), 7.94 (dd, J=8.3, 1.1 Hz, 1H), 7.12 (s, 2H), 6.77 (d, J=6.9 Hz, 2H), 5.03 (s, 2H), 4.24-4.02 (m, 5H), 3.42-3.22 (m, 4H); LC/MS(ESI-MS): [M+H]+=515.2.

### Example 105. 2-(6-(6-(hydroxymethyl)-1H-indazol-1-yl)-2-(4-(4-hydroxyphenyl)piperazin-1-yl)-9H-purin-9-yl)acetic acid

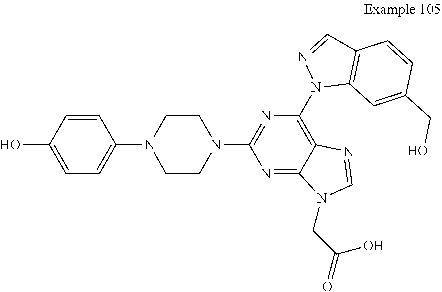 [SMILES: O=C(O)Cn1cnc2c(-n3ncc4ccc(CO)cc43)nc(N3CCN(c4ccc(O)cc4)CC3)nc21]

The proceeding example was prepared according to Example 10 substituting ethyl 1H-indazole-3-carboxylate with (1H-indazol-6-yl)methanol. The desired product 2-(6-(6-(hydroxymethyl)-1H-indazol-1-yl)-2-(4-(4-hydroxyphenyl)piperazin-1-yl)-9H-purin-9-yl)acetic acid was isolated as a white solid.

1H NMR (400 MHz, DMSO-d6): δ 8.74 (s, 1H), 8.58 (s, 1H), 8.46 (s, 1H), 7.88 (d, J=8.2 Hz, 1H), 7.32 (d, J=8.3 Hz, 1H), 7.10 (s, 2H), 6.76 (d, J=8.5 Hz, 2H), 5.04 (s, 2H), 4.75 (s, 3H), 4.18-4.01 (m, 4H), 3.42-3.21 (m, 4H); LC/MS(ESI-MS): [M+H]+=501.2.

### Example 106. 2-(6-(4-chloro-1H-indazol-1-yl)-2-(4-(4-hydroxyphenyl)piperazin-1-yl)-9H-purin-9-yl)acetic acid

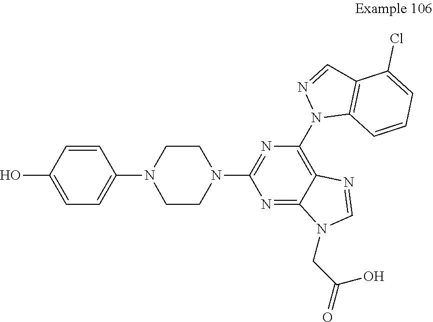 [SMILES: O=C(O)Cn1cnc2c(-n3ncc4c(Cl)cccc43)nc(N3CCN(c4ccc(O)cc4)CC3)nc21]

The proceeding example was prepared according to Example 10 substituting ethyl 1H-indazole-3-carboxylate with 4-chloro-1H-indazole. The desired product 2-(6-(4-chloro-1H-indazol-1-yl)-2-(4-(4-hydroxyphenyl)piperazin-1-yl)-9H-purin-9-yl)acetic acid was isolated as a white solid.

¹H NMR (400 MHz, DMSO-d6): δ 8.68 (s, 1H), 8.55 (d, J=8.5 Hz, 1H), 8.33 (s, 11H), 7.65 (t, J=8.0 Hz, 1H), 7.50 (d, J=7.6 Hz, 1H), 7.08 (s, 2H), 6.75 (d, J=8.6 Hz, 2H), 5.02 (s, 2H), 4.11-4.01 (m, 4H), 3.36-3.22 (m, 4H); LCMS(ESI-MS): [M+H]+=505.1.

### Example 107. 2-(6-(4-methoxy-1H-indazol-1-yl)-2-(4-(4-hydroxyphenyl)piperazin-1-yl)-9H-purin-9-yl)acetic acid

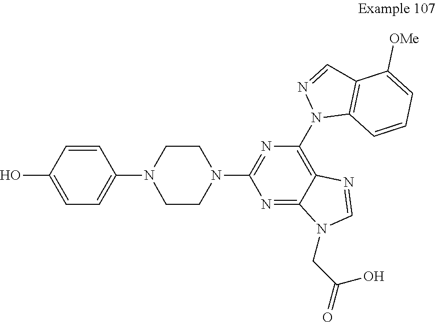 [SMILES: COc1cccc2c1cnn2-c1nc(N2CCN(c3ccc(O)cc3)CC2)nc2c1ncn2CC(=O)O]

The proceeding example was prepared according to Example 10 substituting ethyl 1H-indazole-3-carboxylate with 4-methoxy-1H-indazole. The desired product 2-(6-(4-methoxy-1H-indazol-1-yl)-2-(4-(4-hydroxyphenyl)piperazin-1-yl)-9H-purin-9-yl)acetic acid was isolated as a white solid.

¹H NMR (400 MHz, DMSO-d6): δ 8.58 (s, 1H), 8.44 (s, 1H), 8.16 (d, J=8.4 Hz, 1H), 7.60-7.56 (m, 1H), 7.17-7.01 (m, 2H), 6.75 (d, J=8.8 Hz, 1H), 8.80-8.71 (m, 2H), 5.04 (s, 2H), 4.13-4.01 (m, 4H), 4.01 (s, 3H), 3.44-3.26 (m, 4H), 3.18 (s, 1H); LC/MS(ESI-MS): [M+H]+=501.2.

### Example 108. 2-(6-(4-hydroxy-1H-indazol-1-yl)-2-(4-(4-hydroxyphenyl)piperazin-1-yl)-9H-purin-9-yl)acetic acid

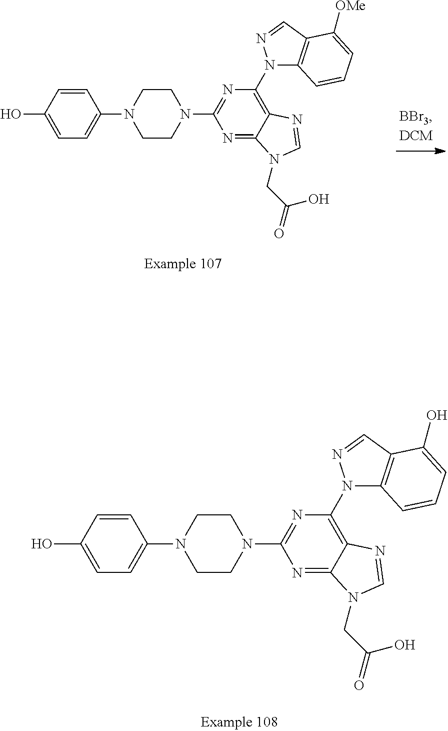 [SMILES: COc1cccc2c1cnn2-c1nc(N2CCN(c3ccc(O)cc3)CC2)nc2c1ncn2CC(=O)O ; O=C(O)Cn1cnc2c(-n3ncc4c(O)cccc43)nc(N3CCN(c4ccc(O)cc4)CC3)nc21]

To a solution of 2-(6-(4-hydroxy-1H-indazol-1-yl)-2-(4-(4-hydroxyphenyl)piperazin-1-yl)-9H-purin-9-yl)acetic acid (50 mg, 0.10 mmol, 1 eq) in DCM (3 mL) at −78° C. was added a solution of BBr3 in DCM (2.08 mL, 2.08 mmol, 20 eq). The resulting mixture was stirred from −78-0° C. over a period of 6 h before it was quenched with water (10 mL). The resulting precipitate was collected and further purified by Prep-HPLC to give 2-(6-(4-hydroxy-1H-indazol-1-yl)-2-(4-(4-hydroxyphenyl)piperazin-1-yl)-9H-purin-9-yl)acetic acid (26 mg) as an off-white solid.

¹H NMR (400 MHz, DMSO-d6): δ 8.57 (s, 1H), 8.42 (s, 1H), 8.04 (d, J=7 Hz, 1H), 7.46-7.42 (m, 1H), 7.14-7.01 (m, 2H), 6.78-6.72 (m, 3H), 5.03 (s, 2H), 4.05 (bs, 4H), 3.27 (bs, 4H); LC/MS(ESI-MS): [M+H]+=487.1.

### Example 109. 2-(6-(5-(hydroxymethyl)-1H-indazol-1-yl)-2-(4-(4-hydroxyphenyl)piperazin-1-yl)-9H-purin-9-yl)acetic acid

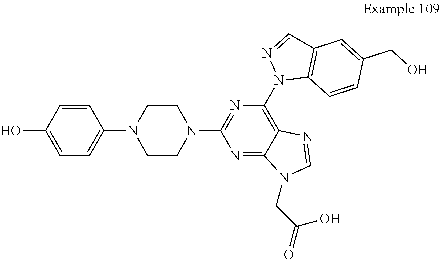 [SMILES: O=C(O)Cn1cnc2c(-n3ncc4cc(CO)ccc43)nc(N3CCN(c4ccc(O)cc4)CC3)nc21]

The proceeding example was prepared according to Example 11 substituting (1H-indazol-3-yl)methanol with (1H-indazol-5-yl)methanol. The desired product 2-(6-(5-(hydroxylmethyl)-1H-indazol-1-yl)-2-(4-(4-hydroxyphenyl)piperazin-1-yl)-9H-purin-9-yl)acetic acid was isolated as a white solid.

¹H NMR (400 MHz, DMSO-d6): δ 8.61 (s, 1H), 8.55 (d, J=8.8 Hz, 1H), 8.49-8.41 (m, 1H), 7.87 (s, 1H), 7.61 (d, J=8.4 Hz, 1H), 7.11-7.01 (m, 2H), 6.76 (d, J=8.8 Hz, 2H), 5.04 (s, 2H), 4.68 (s, 2H), 4.13-3.97 (m, 4H), 3.35-3.30 (m, 4H); LC/MS(ESI-MS): [M+H]+=501.2.

### Example 110. 2-(2-(4-(4-hydroxyphenyl)piperazin-1-yl)-6-methyl-9H-purin-9-yl)propane-1,3-diol

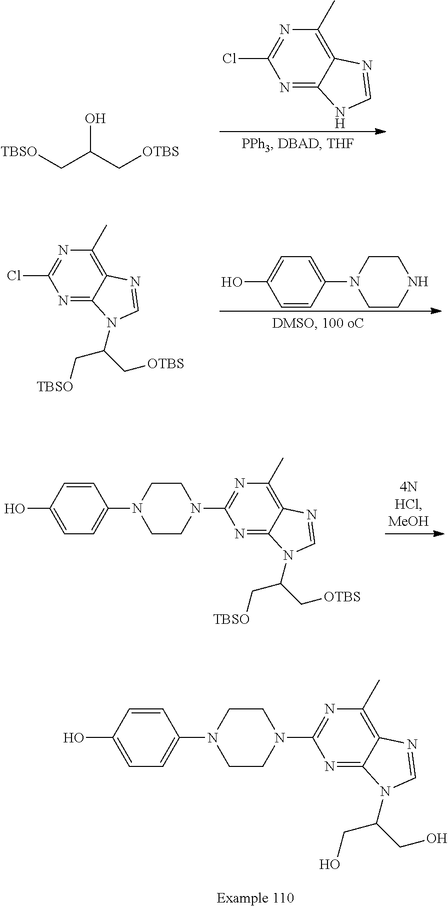 [SMILES: Cc1nc(Cl)nc2[nH]cnc12 ; CC(C)(C)[Si](C)(C)OCC(O)CO[Si](C)(C)C(C)(C)C ; Cc1nc(Cl)nc2c1ncn2C(CO[Si](C)(C)C(C)(C)C)CO[Si](C)(C)C(C)(C)C ; Oc1ccc(N2CCNCC2)cc1 ; Cc1nc(N2CCN(c3ccc(O)cc3)CC2)nc2c1ncn2C(CO[Si](C)(C)C(C)(C)C)CO[Si](C)(C)C(C)(C)C ; Cc1nc(N2CCN(c3ccc(O)cc3)CC2)nc2c1ncn2C(CO)CO]

### Step 1:

To a solution of 2-chloro-6-methyl-9H-purine (0.33 g, 1.95 mmol, 1.0 eq) and 2,2,3,3,9,9,10,10-octamethyl-4,8-dioxa-3,9-disilaundecan-6-ol (941 mg, 2.93 mmol, 1.5 eq) in THF (10 mL) at 0° C. under N2 atmosphere was added PPh3 (0.77 g, 2.93 mmol, 1.5 eq) and DBAD (0.60 g, 2.93 mmol, 1.5 eq). The resulting mixture was stirred from 0-25° C. overnight before the solvent was removed under reduced pressure. The residue was partitioned between EtOAc (100 mL) and water (30 mL). The organic layer was washed with brine, dried over Na₂SO₄ and concentrated. The residue was purified by flash column chromatography on silica gel (petroleum ether:EtOAc=5:1) to give 2-chloro-6-methyl-9-(2,2,3,3,9,9,10,10-octamethyl-4,8-dioxa-3,9-disilaundecan-6-yl)-9H-purine (0.37 g, 41% yield) as a white solid.

### Step 2:

A mixture of 2-chloro-6-methyl-9-(2,2,3,3,9,9,10,10-octamethyl-4,8-dioxa-3,9-di-silaundecan-6-yl)-9H-purine (0.37 g, 0.79 mmol, 1.0 eq) and 1-(4-hydroxyphenyl) piperazine (0.70 g, 3.94 mmol, 5.0 eq) in DMSO (8 mL) was stirred overnight at 100° C. under a nitrogen atmosphere. The reaction mixture was allowed to cool and diluted with water (20 mL). The resulting precipitate was collected by filtration and further purified by Prep-HPLC to give 4-(4-(6-methyl-9-(2,2,3,3,9,9,10,10-octamethyl-4,8-dioxa-3,9-disilaundecan-6-yl)-9H-purin-2-yl)piperazin-1-yl)phenol (0.38 g, 78% yield).

### Step 3:

To a solution of 4-(4-(6-methyl-9-(2,2,3,3,9,9,10,10-octamethyl-4,8-dioxa-3,9-di-silaundecan-6-yl)-9H-purin-2-yl)piperazin-1-yl)phenol (0.38 g, 0.62 mmol, 1 eq) in MeOH (8 mL) was added 4N HCl in MeOH (1.6 mL, 6.21 mmol, 10 eq). The mixture was stirred at room temperature for 0.5 h before it was concentrated. The residue was purified by Prep-HPLC to give 2-(2-(4-(4-hydroxyphenyl)piperazin-1-yl)-6-methyl-9H-purin-9-yl)propane-1,3-diol (168 mg, 71% yield) as a white solid.

¹H NMR (400 MHz, DMSO-d6): δ 8.98 (s, 1H), 7.69-7.81 (m, 2H), 6.90 (d, J=8.8 Hz, 2H), 5.1-4.1 (br, 2H), 3.95-3.61 (m, 10H), 3.17 (s, 1H), 2.67 (s, 3H); LC/MS(ESI-MS): [M+H]+=385.2.

### Example 111. 4-(4-(9-(2-hydroxyethyl)-6-methyl-9H-purin-2-yl)piperazin-1-yl)phenol

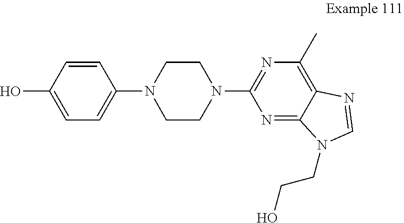 [SMILES: Cc1nc(N2CCN(c3ccc(O)cc3)CC2)nc2c1ncn2CCO]

The proceeding example was prepared according to Example 112 substituting 2,2,3,3,9,9,10,10-octamethyl-4,8-dioxa-3,9-disilaundecan-6-ol with ethylene glycol. The desired product 4-(4-(9-(2-hydroxyethyl)-6-methyl-9H-purin-2-yl)piperazin-1-yl)phenol was isolated as a white solid.

¹H NMR (400 MHz, DMSO-d6): δ 9.41 (bs, 1H), 8.26 (bs, 1H), 8.15 (bs, 1H), 7.43 (d, J=8 Hz, 2H), 7.30 (t, J=8 Hz, 2H), 7.23-6.97 (m, 6H), 6.77 (d, J=8 Hz, 2H), 5.32 (bs, 1H), 4.12 (bs, 2H), 3.90 (bs, 4H), 3.72 (t, J=6 Hz, 2H), 3.21 (bs, 4H), 1.53 (d, J=8 Hz, 3H); LC/MS(ESI-MS): [M+H]+=356.1.

### Example 112. 4-(4-(6-((2-hydroxyethyl)amino)-9-(pyridin-3-yl)-9H-purin-2-yl)piperazin-1-yl)phenol

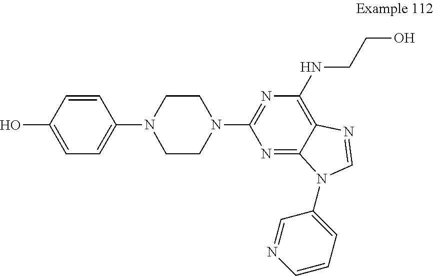 [SMILES: OCCNc1nc(N2CCN(c3ccc(O)cc3)CC2)nc2c1ncn2-c1cccnc1]

The proceeding example was prepared according to Example 90 substituting benzylamine with ethanolamine. The desired product 4-(4-(6-((2-hydroxyethyl)amino)-9-(pyridin-3-yl)-9H-purin-2-yl)piperazin-1-yl)phenol was isolated as a white solid.

¹H NMR (400 MHz, DMSO-d6): δ 9.18 (d, J=2 Hz, 1H), 8.84 (s, 1H), 8.60 (dd, J=1, 4 Hz, 1H), 8.38-8.35 (m, 2H), 7.62 (dd, J=5, 8 Hz, 1H), 7.61 (s, 1H), 6.83 (d, J=9 Hz, 2H), 6.66 (d, J=9 Hz, 2H), 4.74 (t, J=5 Hz, 1H), 3.82 (bs, 4H), 3.63-3.55 (m, 4H), 3.01 (bs, 4H); LC/MS(ESI-MS): [M+H]+=433.2.

### Example 113. 4-(4-(6-((3-hydroxypropyl)amino)-9-(pyridin-3-yl)-9H-purin-2-yl)piperazin-1-yl)phenol

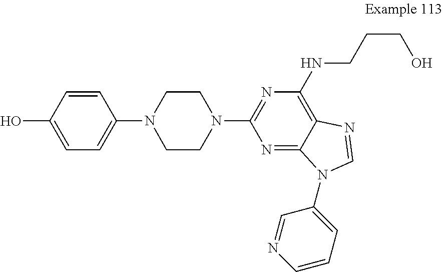 [SMILES: OCCCNc1nc(N2CCN(c3ccc(O)cc3)CC2)nc2c1ncn2-c1cccnc1]

The proceeding example was prepared according to Example 90 substituting benzylamine with 3-aminopropan-1-ol. The desired product 4-(4-(6-((3-hydroxypropyl)-amino)-9-(pyridin-3-yl)-9H-purin-2-yl)piperazin-1-yl)phenol was isolated as a white solid.

1H NMR (400 MHz, DMSO-d6): δ 9.18 (s, 1H), 8.85 (s, 1H), 8.59 (dd, J=1, 4 Hz, 1H), 8.37-8.34 (m, 2H), 7.64-7.61 (m, 2H), 6.84 (d, J=9 Hz, 2H), 6.66 (d, J=9 Hz, 2H), 4.50 (s, 1H), 3.82 (bs, 4H), 3.53-3.48 (m, 3H), 3.01 (bs, 4H), 2.84 (t, J=5 Hz, 1H), 1.78 (m, 2H); LC/MS(ESI-MS): [M+H]+=447.2.

### Example 114. 4-(4-(6-((1-hydroxypropan-2-yl)amino)-9-(pyridin-3-yl)-9H-purin-2-yl)piperazin-1-yl)phenol

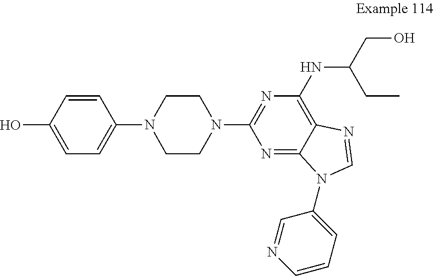 [SMILES: CCC(CO)Nc1nc(N2CCN(c3ccc(O)cc3)CC2)nc2c1ncn2-c1cccnc1]

The proceeding example was prepared according to Example 90 substituting benzylamine with 2-aminopropan-1-ol. The desired product 4-(4-(6-((I-hydroxypropan-2-yl)amino)-9-(pyridin-3-yl)-9H-purin-2-yl)piperazin-1-yl)phenol was isolated as a white solid.

¹H NMR (400 MHz, DMSO-d6): δ 9.18 (s, JH), 8.85 (s, JH), 8.60 (d, J=4 Hz, 1H), 8.37-8.35 (m, 2H), 7.64-7.61 (m, 1H), 7.13-7.11 (m, 1H), 6.83 (d, J=9 Hz, 2H), 6.66 (d, J=9 Hz, 2H), 4.72-4.69 (m, 1H), 4.12 (bs, 1H), 3.82 (bs, 4H), 3.57-3.45 (m, 2H), 3.01 (bs, 4H), 1.75-1.56 (m, 2H), 0.91 (t, J=7 Hz, 3H)); LC/MS(ESI-MS): [M+H]+=461.2.

### Example 115. 4-(4-(6-amino-9-(pyridin-3-yl)-9H-purin-2-yl)piperazin-1-yl)phenol

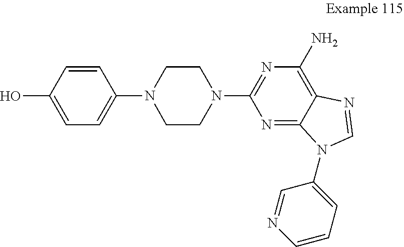 [SMILES: Nc1nc(N2CCN(c3ccc(O)cc3)CC2)nc2c1ncn2-c1cccnc1]

The proceeding example was prepared according to Example 66 substituting phenyl boronic acid with pyridin-3-ylboronic acid. The desired product 4-(4-(6-amino-9-(pyridin-3-yl)-9H-purin-2-yl)piperazin-1-yl)phenol was isolated as a white solid.

¹H NMR (400 MHz, DMSO-d6): δ 9.20 (s, 1H), 8.62 (s, 1H), 8.42-8.37 (m, 2H), 7.67-7.63 (m, 1H), 7.35-7.11 (m, 4H), 6.89-6.79 (m, 2H), 4.02-3.88 (m, 2H), 3.49-3.31 (m, 4H), 2.56-2.51 (m, 2H); LC/MS(ESI-MS): [M+H]+=389.0.

### Example 116. 4-(4-(6-(1H-indazol-1-yl)-9-(pyridin-2-ylmethyl)-9H-purin-2-yl)piperazin-1-yl)phenol

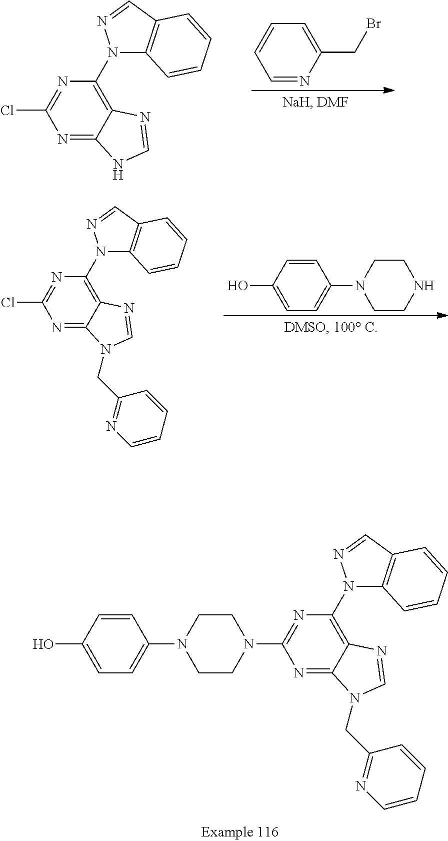

### Step 1:

To a solution of 2-chloro-6-(1H-indazol-1-yl)-9H-purine (400 mg, 1.48 mmol, 1 eq) in DMF (4 mL) at 0° C. was added NaH (71 mg, 1.77 mmol, 60% in oil, 1.2 eq). The resulting mixture was stirred at 0° C. for 15 min and followed by addition of 2-(bromomethyl)pyridine (356 mg, 2.07 mmol, 1.4 eq). The reaction mixture was stirred from 0-25° C. over 6 h before it was quenched with saturated aq. NH₄Cl solution and extracted with EtOAc (75 mL). The organic layer was washed with brine, dried (Na₂SO₄) and concentrated. The crude residue was purified by flash column chromatography on silica gel (5-60% EtOAc in petroleum ether) to provide 2-chloro-6-(1H-indazol-1-yl)-9-(pyridin-2-ylmethyl)-9H-purine (428 mg) as a white solid.

### Step 2:

A mixture of 2-chloro-6-(1H-indazol-1-yl)-9-(pyridin-2-ylmethyl)-9H-purine (0.10 g, 0.28 mmol, 1.0 eq) and 1-(4-hydroxyphenyl) piperazine (246 mg, 1.38 mmol, 5.0 eq) in DMSO (4 mL) was stirred overnight at 100° C. under a nitrogen atmosphere. The reaction mixture was allowed to cool and diluted with water (15 mL). The resulting precipitate was collected by filtration and further purified by Prep-HPLC to give 4-(4-(6-(1H-indazol-1-yl)-9-(pyridin-2-ylmethyl)-9H-purin-2-yl)piperazin-1-yl)phenol (0.12 g) as a white solid.

¹H NMR (400 MHz, DMSO-d6): 5.86 (s, 1H), 8.62-8.52 (m, 3H), 8.28 (s, 1H), 7.94 (d, J=8 Hz, 1H), 7.82 (t, J=8 Hz, 1H), 7.64 (t, J=8 Hz, 1H), 7.40-7.2.7 (m, 3H), 6.86 (d, J=8 Hz, 2H), 6.69 (d, J=8 Hz, 2H), 5.52 (s, 2H), 3.94 (bs, 4H), 3.09 (bs, 4H); LC/MS(ESI-MS): [M+H]+=504.2.

### Example 117. 4-(4-(9-(2-(1H-tetrazol-5-yl)ethyl)-6-(1H-indazol-1-yl)-9H-purin-2-yl)piperazin-1-yl)phenol

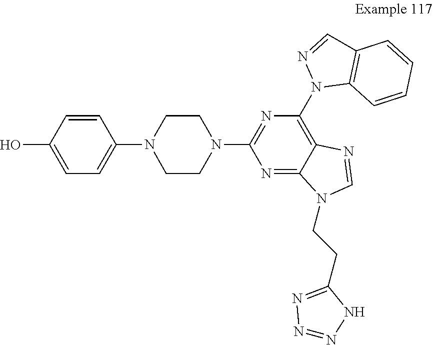 [SMILES: Oc1ccc(N2CCN(c3nc(-n4ncc5ccccc54)c4ncn(CCc5nnn[nH]5)c4n3)CC2)cc1]

The proceeding example was prepared according to Example 17 substituting bromoacetronitrile with 3-bromopropionitrile. The desired product 4-(4-(9-(2-(1H-tetrazol-5-yl)ethyl)-6-(1H-indazol-1-yl)-9H-purin-2-yl)piperazin-1-yl)phenol was isolated as an off-white solid.

¹H NMR (400 MHz, DMSO-d6): δ 8.73 (bs, 1H), 8.68 (s, 1H), 8.59 (d, J=8 Hz, 1H), 7.97 (d, J=8 Hz, 1H), 7.68 (t, J=8 Hz, 1H), 7.45 (t, J=8 Hz, 1H), 7.12 (bs, 2H), 6.77 (d, J=9 Hz, 2H), 4.66 (t, J=7 Hz, 2H), 4.07 (bs, 4H), 3.59 (t, J=7 Hz, 2H), 3.33 (bs, 4H); LC/MS(ESI-MS): [M+H]+=509.2.

### Example 118. 2-(2-(4-(4-hydroxyphenyl)piperazin-1-yl)-6-(1H-indol-4-yl)-9H-purin-9-yl)acetic acid

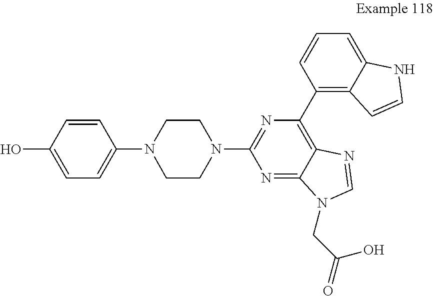 [SMILES: O=C(O)Cn1cnc2c(-c3cccc4[nH]ccc34)nc(N3CCN(c4ccc(O)cc4)CC3)nc21]

The proceeding example was prepared according to Example 34 substituting (1H-indazol-4-yl)boronic acid with (1H-indol-4-yl)boronic acid. The desired product 2-(2-(4-(4-hydroxyphenyl)piperazin-1-yl)-6-(1H-indol-4-yl)-9H-purin-9-yl)acetic acid was isolated as an off-white solid.

¹H NMR (400 MHz, DMSO-d6): δ 11.33 (s, 1H), 8.54 (d, J=7 Hz, 1H), 8.21 (s, 1H), 7.60 (d, J=8 Hz, 1H), 7.50 (d, J=3 Hz, 1H), 7.49 (s, 1H), 7.24-7.11 (m, 3H), 6.76 (d, J=8 Hz, 2H), 5.00 (s, 2H), 4.05 (bs, 4H), 3.31 (bs, 4H); LC/MS(ESI-MS): [M+H]+=470.1.

### Example 119. 2-(2-(4-(4-hydroxyphenyl)piperazin-1-yl)-6-(1-oxoisoindolin-2-yl)-9H-purin-9-yl)acetic acid

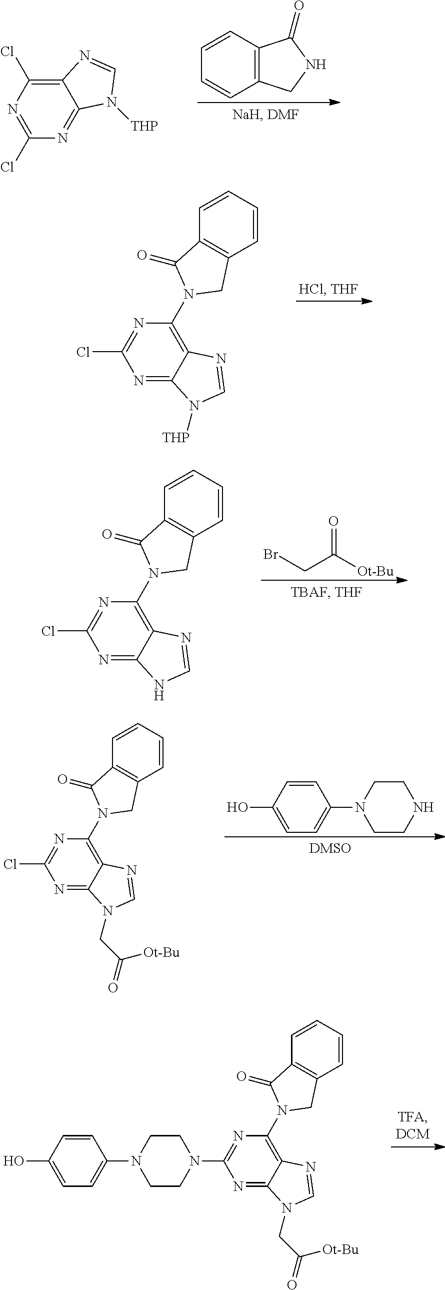

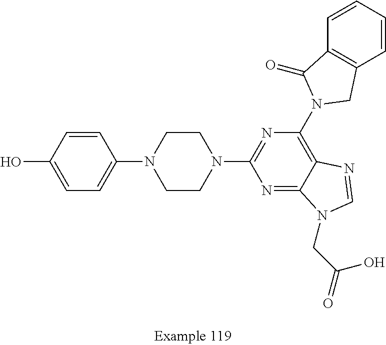 [SMILES: O=C(O)Cn1cnc2c(N3Cc4ccccc4C3=O)nc(N3CCN(c4ccc(O)cc4)CC3)nc21]

### Step 1:

To a solution of isoindolin-1-one (244 mg, 1.83 mmol, 1.0 eq) in DMF (5 mL) at 0° C. was added NaH (88 mg, 2.20 mmol, 1.2 eq, 60% in oil) in portions. The mixture was stirred for 0.5 h and followed by addition of 2,6-dichloro-9-(tetrahydro-2H-pyran-2-yl)-9H-purine (500 mg, 1.83 mmol, 1.0 eq). The mixture was stirred for at room temperature before it was diluted with water (20 mL). The resulting precipitate was collected by filtration. The solid was dissolved in a mixture of THF (5 mL) and 6N aq. HCl (2 mL). The reaction mixture was stirred for 30 min as precipitate was formed. The solid was collected by filtration and dried to give 2-(2-chloro-9H-purin-6-yl)isoindolin-1-one (150 mg, 29% yield).

### Step 2:

To a solution of 2-(2-chloro-9H-purin-6-yl)isoindolin-1-one (150 mg, 0.52 mmol, 1 eq) in THF (5 mL) at 0° C. was added a solution of 2M TBAF in THF (1.05 mL, 1.05 mmol, 2 eq) dropwise. The mixture was stirred for 0.5 h and followed by addition of tert-butyl 2-bromoacetate (205 mg, 1.05 mmol, 2 eq). The mixture was stirred at room temperature overnight before it was concentrated. The residue was partitioned between EtOAc (50 mL) and water (20 mL). The organic layer was washed with brine, dried Na₂SO₄, and concentrated. The residue was purified by flash column chromatography on silica gel (petroleum ether:EtOAc=4:6) to give tert-butyl 2-(2-chloro-6-(1-oxoisoindolin-2-yl)-9H-purin-9-yl)acetate (170 mg, 91% yield).

### Step 3:

A mixture of tert-butyl 2-(2-chloro-6-(1-oxoisoindolin-2-yl)-9H-purin-9-yl)acetate (170 mg, 0.42 mmol, 1 eq) and 4-(piperazin-1-yl)phenol (379 mg, 3.12 mmol, 5.0 eq) in DMSO (5 mL) under nitrogen atmosphere was stirred at 100° C. for 12 h before it was allowed to cool and diluted with water (20 mL). The resulting precipitate was collected by filtration and dried to give crude tert-butyl 2-(2-(4-(4-hydroxyphenyl)piperazin-1-yl)-6-(1-oxoisoindolin-2-yl)-9H-purin-9-yl)acetate (200 mg) which was used directly in next step without further purification.

### Step 4:

A mixture of crude tert-butyl 2-(2-(4-(4-hydroxyphenyl)piperazin-1-yl)-6-(1-oxoisoindolin-2-yl)-9H-purin-9-yl)acetate (120 mg, 0.221 mmol, 1.0 eq) and TFA (0.5 mL) in DCM (2 mL) was stirred at room temperature for 6 h. The resulting precipitate was collected by filtration and further purified by Prep-HPLC to give 2-(2-(4-(4-hydroxyphenyl)-piperazin-1-yl)-6-(1-oxoisoindolin-2-yl)-9H-purin-9-yl)acetic acid (11 mg, 9% yield) as a white solid.

¹H NMR (400 MHz, DMSO-d6): δ 9.31 (bs, 1H), 8.57 (s, 1H), 7.89 (d, J=8 Hz, 1H), 7.80 (bs, 2H), 7.64-7.61 (m, 1H), 7.09 (bs, 2H), 6.76 (d, J=8 Hz 2H), 5.39 (s, 2H), 5.04 (s, 2H), 4.04 (bs, 4H), 3.27 (bs, 4H); LC/MS(ESI-MS): [M+H]+=4861.

### Example 120. 2-(6-(3H-[1,2,3]triazolo[4,5-b]pyridin-3-yl)-2-(4-(4-hydroxyphenyl)piperazin-1-yl)-9H-purin-9-yl)acetic acid

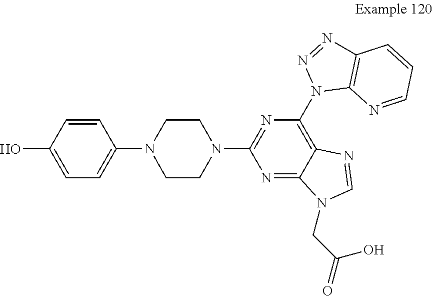 [SMILES: O=C(O)Cn1cnc2c(-n3nnc4cccnc43)nc(N3CCN(c4ccc(O)cc4)CC3)nc21]

The proceeding example was prepared according to Example 24 substituting 1H-benzo[d][1,2,3]triazole with 3H-[1,2,3]triazolo[4,5-b]pyridine. The desired product 2-(6-(3H-[1,2,3]triazolo[4,5-b]pyridin-3-yl)-2-(4-(4-hydroxyphenyl)piperazin-1-yl)-9H-purin-9-yl)acetic acid was isolated as an off-white solid.

¹H NMR (400 MHz, DMSO-d6): δ 8.90-8.88 (m, 2H), 8.38 (s, 1H), 7.85-7.82 (m, 1H), 7.13-7.01 (m, 2H), 6.78-6.74 (m, 2H), 5.06 (s, 2H), 4.13-4.03 (m, 4H), 3.44-3.41 (m, 4H); LC/MS(ESI-MS): [M+H]+=471.1.

### Example 121. 2-(6-(3H-[1,2,3]triazolo[4,5-c]pyridin-3-yl)-2-(4-(4-hydroxyphenyl)piperazin-1-yl)-9H-purin-9-yl)acetic acid

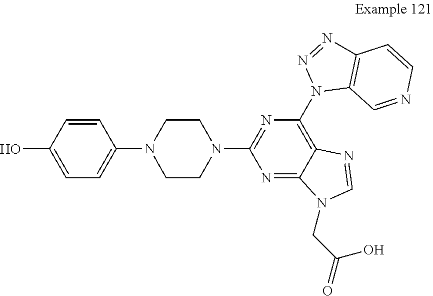 [SMILES: O=C(O)Cn1cnc2c(-n3nnc4ccncc43)nc(N3CCN(c4ccc(O)cc4)CC3)nc21]

The proceeding example was prepared according to Example 24 substituting 1H-benzo[d][1,2,3]triazole with 3H-[1,2,3]triazolo[4,5-c]pyridine. The desired product 2-(6-(3H-[1,2,3]triazolo[4,5-c]pyridin-3-yl)-2-(4-(4-hydroxyphenyl)piperazin-1-yl)-9H-purin-9-yl)acetic acid was isolated as an off-white solid.

¹H NMR (400 MHz, DMSO-d6): δ 9.70 (s, 1H), 8.80 (d, J=6 Hz, 1H), 8.42 (d, J=5 Hz, 1H), 8.38 (s, 1H), 7.08 (bs, 2H), 6.76 (d, J=8 Hz, 2H), 5.06 (s, 2H), 4.08 (bs, 4H), 3.31 (bs, 4H); LC/MS(ESI-MS): [M+H]+=473.8.

### Example 122. 1-(9-(carboxymethyl)-2-(4-(4-hydroxyphenyl)piperazin-1-yl)-9H-purin-6-yl)-1H-benzo[d][1,2,3]triazole-4-carboxylic acid

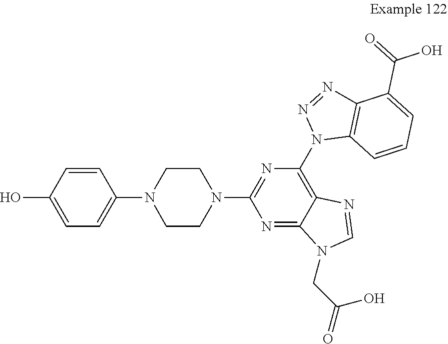 [SMILES: O=C(O)Cn1cnc2c(-n3nnc4c(C(=O)O)cccc43)nc(N3CCN(c4ccc(O)cc4)CC3)nc21]

The proceeding example was prepared according to Example 24 substituting 1H-benzo[d][1,2,3]triazole with ethyl 1H-benzo[d][1,2,3]triazole-4-carboxylate. The desired product 1-(9-(carboxymethyl)-2-(4-(4-hydroxyphenyl)piperazin-1-yl)-9H-purin-6-yl)-1H-benzo[d][1,2,3]triazole-4-carboxylic acid was isolated as an off-white solid.

¹H NMR (400 MHz, DMSO-d6): δ 8.68 (d, J=8 Hz, 1H), 8.36 (s, 1H), 8.11 (d, J=8 Hz, 1H), 7.86 (t, J=8 Hz, 1H), 7.10 (bs, 2H), 6.74 (d, J=9 Hz, 2H), 5.06 (s, 2H), 4.06 (bs, 4H), 3.17 (bs, 4H); LC/MS(ESI-MS): [M+H]+=516.1.

### Example 123. 2-(6-(4-(hydroxymethyl)-1H-benzo[d][1,2,3]triazol-1-yl)-2-(4-(4-hydroxyphenyl)piperazin-1-yl)-9H-purin-9-yl)acetic acid

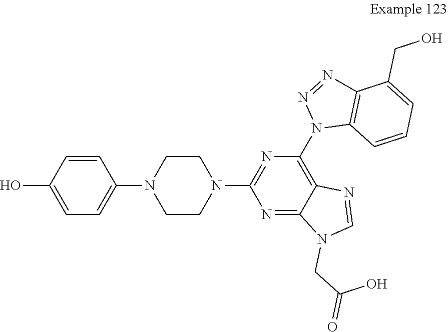 [SMILES: O=C(O)Cn1cnc2c(-n3nnc4c(CO)cccc43)nc(N3CCN(c4ccc(O)cc4)CC3)nc21]

The proceeding example was prepared according to Example 16 substituting (1H-indazol-4-yl)methanol with (1H-benzo[d][1,2,3]triazol-4-yl)methanol. The desired product 2-(6-(4-(hydroxymethyl)-1H-benzo[d][1,2,3]triazol-1-yl)-2-(4-(4-hydroxyphenyl)piperazin-1-yl)-9H-purin-9-yl)acetic acid was isolated as an off-white solid.

¹H NMR (400 MHz, DMSO-d6): δ 8.34-8.31 (m, 2H), 7.76 (t, J=8 Hz, 1H), 7.61 (d, J=14 Hz, 1H), 7.10 (bs, 2H), 6.75 (d, J=7 Hz, 2H), 5.17 (s, 2H), 5.05 (s 2H), 4.08 (bs, 4H), 3.31 (bs, 4H); LC/MS(ESI-MS): [M+H]+=502.1.

### Examples 124. 2-(2-(4-(4-hydroxyphenyl)piperazin-1-yl)-6-(1H-indazol-1-yl)-9H-purin-9-yl)propanoic acid

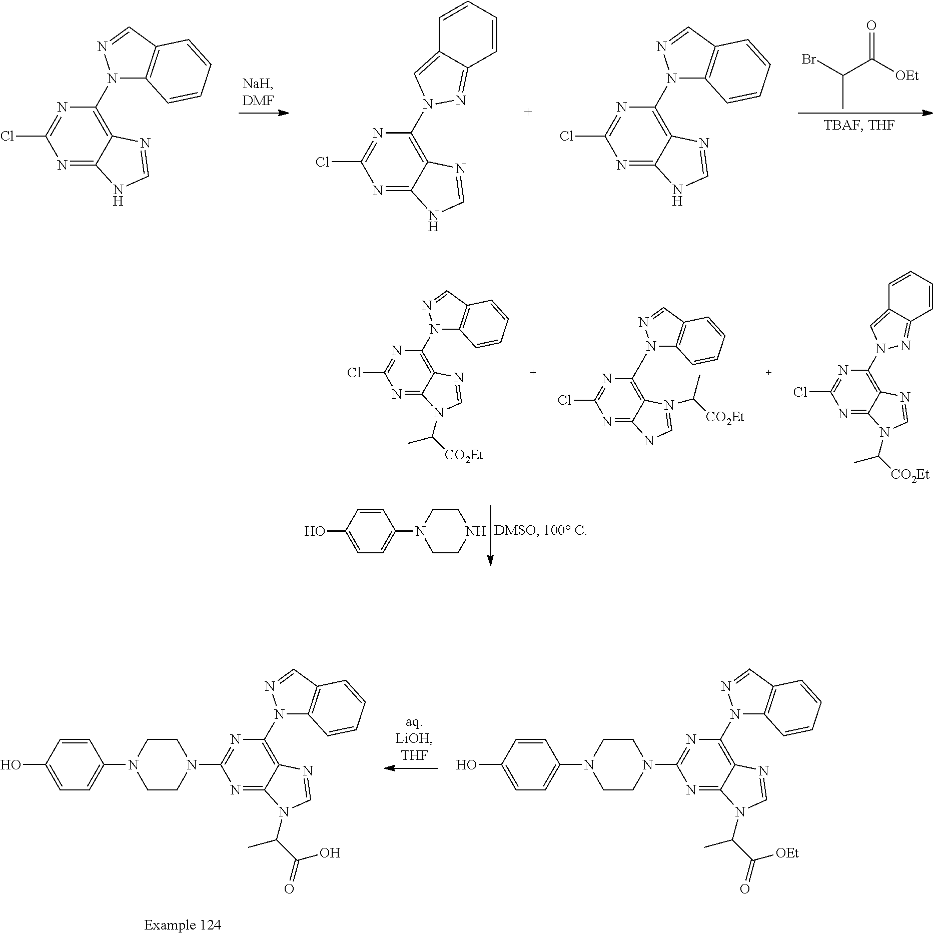

### Step 1:

To a solution of indazole (6.25 g, 52.90 mmol, 1 eq) in DMF (20 mL) at 0° C. under nitrogen atmosphere was added sodium hydride (4.23 g, 0.11 mol, 60% in mineral oil, 2 eq) in portions. The mixture was stirred for 0.5 h and followed by addition of 2,6-dichloro-9H-purine (10 g, 52.91 mmol, 1.0 eq) in DMF (80 mL). Then the resulting mixture was stirred at 80° C. overnight before it was allowed to cool and diluted with water (50 mL) and the resulting precipitate was collected by filtration and dried to give a crude mixture of 2-chloro-6-(1H-indazol-1-yl)-9H-purine and 2-chloro-6-(1H-indazol-1-yl)-9H-purine (ca. 10:1 ratio, 4.7 g, 34% yield) of as an off-white solid.

### Step 2:

To a mixture of 2-chloro-6-(1H-indazol-1-yl)-9H-purine (0.7 g, 2.59 mmol, 1.0 eq) in THF (15 mL) at 0° C. was added a solution of 1M TBAF in THF (6 mL, 5.80 mmol, 2.0 eq) and ethyl 2-bromopropanoate (1.50 g, 5.80 mmol, 1.0 eq). The resulting mixture was stirred at 25° C. overnight before it was diluted with water (30 mL), extracted with EtOAc (3×30 mL). The combined organic layer was washed with brine, dried over Na₂SO₄ and concentrated. The crude product was then purified by flash column chromatography on silica gel (petroleum ether:EtOAc=1:1) to give ethyl 2-(2-chloro-6-(1H-indazol-1-yl)-9H-purin-9-yl)propanoate (0.36 g), ethyl 2-(2-chloro-6-(1H-indazol-1-yl)-7H-purin-7-yl)propanoate (0.14 g), and ethyl 2-(2-chloro-6-(2H-indazol-2-yl)-9H-purin-9-yl)propanoate (0.16 g). All were isolated as white solids.

### Step 3:

A mixture of ethyl 2-(2-chloro-6-(1H-indazol-1-yl)-9H-purin-9-yl)propanoate (0.36 g, 0.97 mmol, 1.0 eq) and 1-(4-hydroxyphenyl) piperazine (0.52 g, 2.93 mmol, 3 eq) in DMSO (8 mL) under nitrogen atmosphere was stirred at 100° C. overnight. The mixture was allowed to cool and diluted with water (30 mL) and the resulting precipitate was collected by filtration and dried to give crude ethyl 2-(2-(4-(4-hydroxyphenyl)piperazin-1-yl)-6-(1H-indazol-1-yl)-9H-purin-9-yl)propanoate (0.4 g, 80% yield) as an off-white solid.

### Step 4:

To a solution of crude ethyl 2-(2-(4-(4-hydroxyphenyl)piperazin-1-yl)-6-(1H-indazol-1-yl)-9H-purin-9-yl)propanoate (0.4 g, 0.78 mmol, 1 eq) in THF (4 mL) and H₂O (4 mL) was added LiOH (0.16 g, 3.91 mmol, 5 eq). The mixture was stirred at 25° C. for 1 h before the organic volatile was removed. The resulting aq. phase was diluted with water (10 mL) and adjusted to pH 4 with 1N aq. HCl solution. The resulting precipitate was collected by filtration. The crude was purified by Prep-HPLC to give 2-(2-(4-(4-hydroxyphenyl)-piperazin-1-yl)-6-(1H-indazol-1-yl)-9H-purin-9-yl)propanoic acid (100 mg, 26% yield) as a white solid.

¹H NMR (400 MHz, DMSO-d6): δ 8.93-8.53 (m, 3H), 7.98 (d, J=8.0 Hz, 1H), 7.67 (t, J=7.5 Hz, 1H), 7.44 (t, J=7.5 Hz, 1H), 7.14 (bs, 2H), 6.78 (d, J=8.2 Hz, 2H), 5.52-5.35 (m, 1H), 4.29-3.92 (m, 4H), 3.53-3.22 (m, 4H), 1.87 (d, J=7.4 Hz, 3H); LCMS(ESI-MS): [M+H]+=485.2.

### Examples 125. 2-(2-(4-(4-hydroxyphenyl)-piperazin-1-yl)-6-(1H-indazol-1-yl)-7H-purin-7-yl)propanoic acid

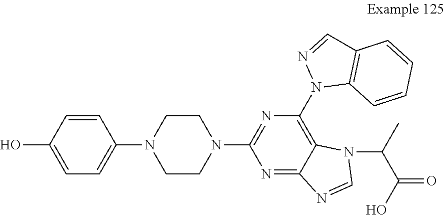 [SMILES: CC(C(=O)O)n1cnc2nc(N3CCN(c4ccc(O)cc4)CC3)nc(-n3ncc4ccccc43)c21]

The proceeding example was prepared according to Example 124 substituting ethyl 2-(2-chloro-6-(1H-indazol-1-yl)-9H-purin-9-yl)propanoate with ethyl 2-(2-chloro-6-(1H-indazol-1-yl)-7H-purin-7-yl)propanoate. The desired product 2-(2-(4-(4-hydroxyphenyl)-piperazin-1-yl)-6-(1H-indazol-1-yl)-7H-purin-7-yl)propanoic acid was isolated as an off-white solid.

¹H NMR (400 MHz, DMSO-d6): δ 9.75 (s, 1H), 8.55 (s, 1H), 7.87 (d, J=8 Hz, 1H), 7.78 (t, J=9 Hz, 1H), 7.38 (t, J=7 Hz, 1H), 7.17-7.14 (m, 3H), 6.79 (d, J=8 Hz, 2H), 5.43-5.40 (m, 1H), 4.12 (bs, 4H), 3.38 (bs, 4H), 1.87 (d, J=8 Hz, 3H); LC/MS(ESI-MS): [M+H]+=485.2.

### Examples 126. 2-(2-(4-(4-hydroxyphenyl)piperazin-1-yl)-6-(2H-indazol-2-yl)-9H-purin-9-yl)propanoic acid

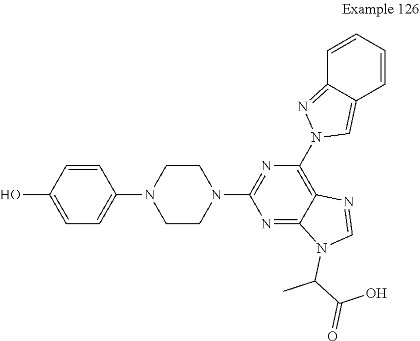 [SMILES: CC(C(=O)O)n1cnc2c(-n3cc4ccccc4n3)nc(N3CCN(c4ccc(O)cc4)CC3)nc21]

The proceeding example was prepared according to Example 124 substituting ethyl 2-(2-chloro-6-(1H-indazol-1-yl)-9H-purin-9-yl)propanoate with ethyl 2-(2-chloro-6-(2H-indazol-2-yl)-9H-purin-9-yl)propanoate. The desired product 2-(2-(4-(4-hydroxyphenyl)piperazin-1-yl)-6-(2H-indazol-2-yl)-9H-purin-9-yl)propanoic acid was isolated as an off-white solid.

¹H NMR (400 MHz, DMSO-d6): δ 8.74 (s, 1H), 8.53 (s, 1H), 8.35 (d, J=8 Hz, 1H), 7.98 (d, J=8 Hz, 1H), 7.63 (t, J=8 Hz, 1H), 7.41 (t, J=8 Hz, 1H), 7.25 (bs, 2H), 6.80 (d, J=8 Hz, 2H), 5.86-5.75 (m, 1H), 4.05 (bs, 4H), 3.43 (bs, 4H), 1.83 (d, J=8 Hz, 3H); LC/MS(ESI-MS): [M+H]+=485.2.

### Example 127. 2-(2-(4-(4-hydroxyphenyl)piperazin-1-yl)-6-(1H-indazol-1-yl)-9H-purin-9-yl)butanoic acid

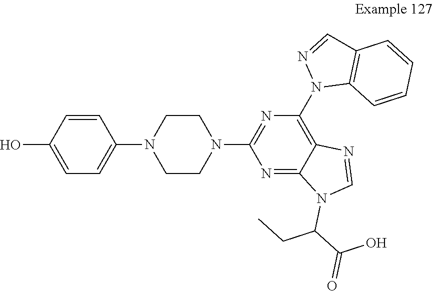 [SMILES: CCC(C(=O)O)n1cnc2c(-n3ncc4ccccc43)nc(N3CCN(c4ccc(O)cc4)CC3)nc21]

The proceeding example was prepared according to Example 124 substituting ethyl 2-bromoacetate with ethyl 2-bromopropionate. The desired product 2-(2-(4-(4-hydroxy-phenyl)piperazin-1-yl)-6-(1H-indazol-1-yl)-9H-purin-9-yl)butanoic acid was isolated as an off-white solid.

¹H NMR (400 MHz, DMSO-d6): δ 9.18-9.17 (m, 1H), 8.72-8.70 (m, 1H), 8.60 (s, 1H), 7.97 (d, J=8 Hz, 1H), 7.69 (d, J=8 Hz, 1H), 7.41-7.38 (m, 2H), 6.92 (d, J=9 Hz, 2H), 5.49-5.45 (m, 1H), 4.38 (bs, 4H), 3.69 (bs, 4H), 2.51-2.41 (m, 2H), 1.06 (t, J=7 Hz, 3H); LC/MS(ESI-MS): [M+H]+=498.9.

### Example 128. 2-(2-(4-(4-hydroxyphenyl)piperazin-1-yl)-6-(1H-indazol-1-yl)-9H-purin-9-yl)-2-methylpropanoic acid

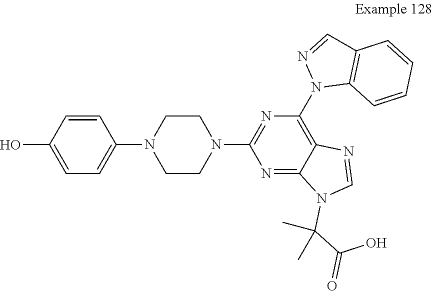 [SMILES: CC(C)(C(=O)O)n1cnc2c(-n3ncc4ccccc43)nc(N3CCN(c4ccc(O)cc4)CC3)nc21]

The proceeding example was prepared according to Example 124 substituting ethyl 2-bromoacetate with ethyl-bromoisobutyrate. The desired product 2-(2-(4-(4-hydroxy-phenyl)piperazin-1-yl)-6-(1H-indazol-1-yl)-9H-purin-9-yl)-2-methylpropanoic acid was isolated as an off-white solid.

¹H NMR (400 MHz, DMSO-d6): δ 9.15 (s, 1H), 8.72 (s, 1H), 8.60 (s, 1H), 7.98 (d, J=8 Hz, 1H), 7.72 (t, J=8 Hz, 1H), 7.49 (t, J=8 Hz, 1H), 7.34-7.32 (m, 2H), 6.90 (d, J=9 Hz, 2H), 4.32 (bs, 4H), 3.61 (bs, 4H), 2.08 (s, 6H); LC/MS(ESI-MS): [M+H]+=498.9.

### Example 129. 2-(2-(4-(6-hydroxypyridin-3-yl)piperazin-1-yl)-6-(1H-indazol-1-yl)-9H-purin-9-yl)acetic acid

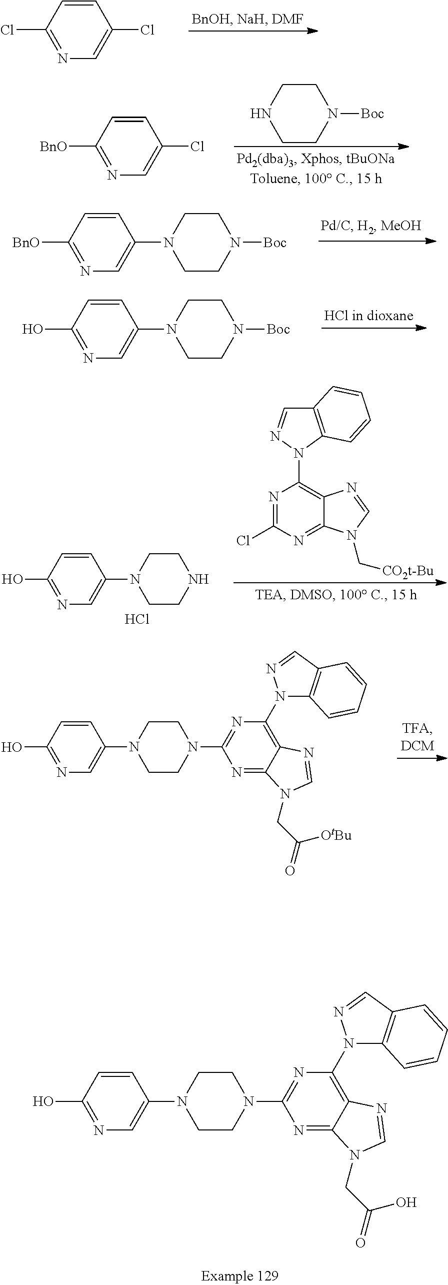

### Step 1:

To a solution of 2,5-dichloropyridine (2 g, 13.51 mmol, 1.0 eq) in DMF (30 mL) under nitrogen atmosphere at 0° C. was added sodium hydride (0.65 g, 16.21 mmol, 1.2 eq) in portions. The mixture was stirred for 0.5 h and followed by addition of a solution of BnOH (1.75 g, 16.21 mmol, 1.2 eq) in DMF (2 mL). Then the resulting mixture was stirred at 5° C. overnight before it was diluted with water (90 mL) and extracted with EtOAc (3×90 mL). The combined organic layer was washed with brine, dried over Na₂SO₄, and concentrated. The crude residue was purified by flash column chromatography on silica gel (petroleum ether:EtOAc=100:1) to give 2-(benzyloxy)-5-chloropyridine (2.7 g, 91% yield).

### Step 2:

To a solution of 2-(benzyloxy)-5-chloropyridine (1.5 g, 6.85 mmol, 1.0 eq) in toluene (15 mL) under nitrogen atmosphere was added NaOtBu (1.98 g, 20.55 mmol, 3.0 eq), 1-Boc-piperazine (1.53 g, 8.22 mmol, 1.2 eq), X-phos (0.65 g, 1.37 mmol, 0.2 eq) and Pd2(dba)3 (0.63 g, 0.69 mmol, 0.1 eq). The mixture was stirred at 110° C. overnight before it was allowed to cool and concentrated. The residue was diluted with water (30 mL) and extracted with EtOAc (3×30 mL). The combined organic layer was washed with brine, dried over Na₂SO₄, and concentrated. The crude residue was purified by flash column chromatography on silica gel (petroleum ether:EtOAc=5:1) to give of tert-butyl 4-(6-(benzyloxy)pyridin-3-yl)piperazine-1-carboxylate (0.8 g, 32% yield) as an oil.

### Step 3:

A mixture of tert-butyl 4-(6-(benzyloxy)pyridin-3-yl)piperazine-1-carboxylate (0.8 g, 2.17 mmol, 1.0 eq) and 10% Pd/C catalyst (0.3 g) in methanol (5 mL) under 1 atm of H₂ was stirred at 25° C. overnight. The catalyst was filtered off and the filtrate was concentrated to give tert-butyl 4-(6-hydroxypyridin-3-yl)piperazine-1-carboxylate (0.5 g, 84% yield).

### Step 4:

To a solution of tert-butyl 4-(6-hydroxypyridin-3-yl)piperazine-1-carboxylate (0.5 g, 1.79 mmol, 1 eq) in DCM (3 mL) at 25° C. was added a solution of 4M HCl in dioxane (4.5 mL, 17.92 mmol, 10 eq). The mixture was stirred for 1 h before it was concentrated to give 5-(piperazin-1-yl)pyridin-2-ol (0.3 g, 93% yield) as an off-white solid.

### Step 5:

To a mixture of 5-(piperazin-1-yl)pyridin-2-ol (0.3 g, 1.69 mmol, 2.5 eq) and tert-butyl 2-(2-chloro-6-(1H-indazol-1-yl)-9H-purin-9-yl)acetate (0.26 g, 0.67 mmol, 1 eq) in DMSO (3 mL) under nitrogen atmosphere was added Et3N (0.68 g, 16.9 mmol, 10 eq). The mixture was stirred at 100° C. overnight before it was allowed to cool and diluted with water (20 mL) and the resulting solid was collected by filtration and dried to give tert-butyl 2-(2-(4-(6-hydroxypyridin-3-yl)piperazin-1-yl)-6-(1H-indazol-1-yl)-9H-purin-9-yl)acetate (0.14 g, 16% yield) as an oil.

### Step 6:

A mixture of tert-butyl 2-(2-(4-(6-hydroxypyridin-3-yl)piperazin-1-yl)-6-(1H-indazol-1-yl)-9H-purin-9-yl)acetate (0.14 g, 0.27 mmol, 1 eq) and TFA (3 mL) in DCM (3 mL) was stirred at room temperature for 0.5 h. The mixture was concentrated under reduced pressure. The residue was purified by Prep-HPLC to give 2-(2-(4-(6-hydroxypyridin-3-yl)piperazin-1-yl)-6-(1H-indazol-1-yl)-9H-purin-9-yl)acetic acid (82 mg, 65% yield) as a white solid.

¹H NMR (400 MHz, DMSO-d6): δ 8.65 (s, 1H), 8.61 (d, J=8.5 Hz, 1H), 8.57 (s, 1H), 7.98 (d, J=8.0 Hz, 1H), 7.72-7.56 (m, 2H), 7.44 (t, J=7.5 Hz, 1H), 6.97 (s, 1H), 6.45 (d, J=9.4 Hz, 1H), 5.04 (s, 2H), 4.05-3.94 (m, 4H), 3.12-2.99 (m, 4H); LC/MS(ESI-MS): [M+H]+=472.1.

### Example 130. 2-(2-(4-(3-fluoro-5-hydroxypyridin-2-yl)piperazin-1-yl)-6-(1H-indazol-1-yl)-9H-purin-9-yl)acetic acid 923

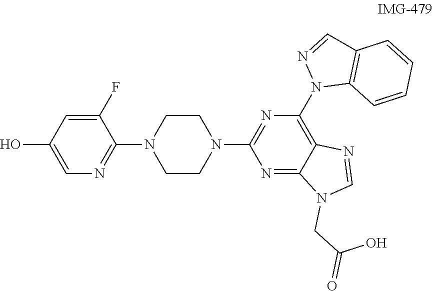 [SMILES: O=C(O)Cn1cnc2c(-n3ncc4ccccc43)nc(N3CCN(c4ncc(O)cc4F)CC3)nc21]

The proceeding example was prepared according to Example 21 substituting 6-bromopyridin-3-ol with 6-chloro-5-fluoropyridin-3-ol. The desired 2-(2-(4-(3-fluoro-5-hydroxypyridin-2-yl)piperazin-1-yl)-6-(1H-indazol-1-yl)-9H-purin-9-yl)acetic acid was isolated as an off-white solid.

¹H NMR (400 MHz, DMSO-d6): δ 9.78 (br s, 1H), 8.71-8.53 (m, 3H), 7.97 (d, J=7.9 Hz, 1H), 7.76-7.66 (m, 2H), 7.44 (t, J=7.5 Hz, 1H), 7.11 (dd, J=13.5, 2.2 Hz, 1H), 5.05 (s, 2H), 4.07-3.95 (m, 4H), 3.39-3.28 (m, 4H); LC/MS(ESI-MS): [M+H]+=[M+H]+=489.8.

### Example 131. methyl 2-(2-(4-(4-hydroxyphenyl)piperazin-1-yl)-6-(1H-indazol-1-yl)-9H-purin-9-yl)acetate

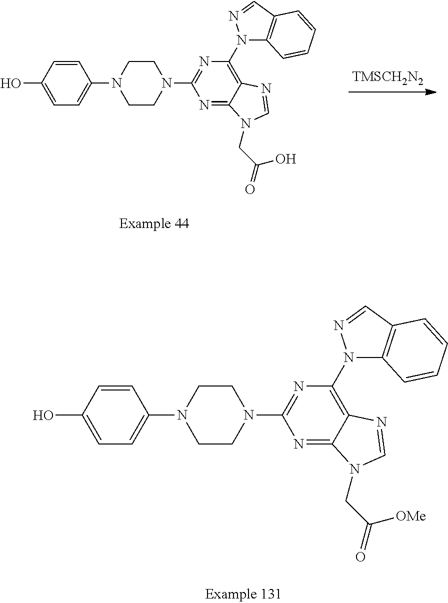 [SMILES: O=C(O)Cn1cnc2c(-n3ncc4ccccc43)nc(N3CCN(c4ccc(O)cc4)CC3)nc21 ; COC(=O)Cn1cnc2c(-n3ncc4ccccc43)nc(N3CCN(c4ccc(O)cc4)CC3)nc21]

To a solution of Example 44 (65 mg, 0.14 mmol, 1 eq) in DCM (5 mL) at 25° C. was added a solution 2M TMSCH₂N₂ in hexanes (207 L, 0.41 mmol, 3 eq). The mixture was stirred for 1 h before it was concentrated to provide the desired product (67 mg) as an off-white solid.

¹H NMR (400 MHz, DMSO-d6): δ 8.64 (s, JH), 8.60 (d, J=8.5 Hz, 1H), 8.45 (s, 1H), 7.97 (d, J=7.9 Hz, 1H), 7.65 (t, J=7.7 Hz, 1H), 7.43 (t, J=7.5 Hz, 1H), 7.18 (s, 2H), 6.79 (d, J=8.7 Hz, 2H), 5.17 (s, 2H), 4.25-3.94 (m, 4H), 3.75 (s, 3H), 3.47-3.32 (m, 4H); LC/MS(ESI-MS): [M+H]+=485.2.

### Example 132. 2-(2-(2-(4-(4-hydroxyphenyl)piperazin-1-yl)-6-(1H-indazol-1-yl)-9H-purin-9-yl)acetamido)acetic acid

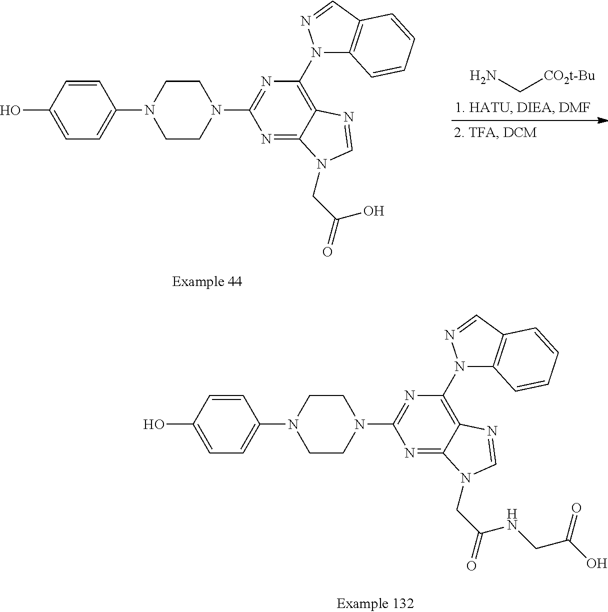 [SMILES: O=C(O)Cn1cnc2c(-n3ncc4ccccc43)nc(N3CCN(c4ccc(O)cc4)CC3)nc21 ; CC(C)(C)OC(=O)CN ; O=C(O)CNC(=O)Cn1cnc2c(-n3ncc4ccccc43)nc(N3CCN(c4ccc(O)cc4)CC3)nc21]

### Step 1:

To a solution of Example 44 (100 mg, 0.21 mmol, 1.0 eq) in DMF (4 mL) at 0° C. was added glycine tert-butyl ester (36 mg, 0.28 mmol, 1.3 eq), DIEA (185 mL, 1.06 mmol, 5.0 eq) and HATU (105 mg, 0.28 mmol, 1.3 eq). The mixture was stirred for 24 h before it was diluted with water (4 mL) and extracted with EtOAc (15 mL). The organic layer was washed with water (5 mL), brine (2 mL), dried over Na₂SO₄ and concentrated. The crude was purified by flash column chromatography on silica gel (0-5% MeOH in DCM) to provide methyl 2-(2-(2-(4-(4-hydroxyphenyl)piperazin-1-yl)-6-(1H-indazol-1-yl)-9H-purin-9-yl)acetamido)acetate (108 mg) as an off-white solid.

### Step 2:

A mixture of (108 mg, 0.20 mmol, 1 eq) and TFA (3 mL) in DCM (3 mL) at 25° C. was stirred for 4 h before it was concentrated under reduced pressure. The crude was purified by HPLC to 2-(2-(2-(4-(4-hydroxyphenyl)piperazin-1-yl)-6-(1H-indazol-1-yl)-9H-purin-9-yl)acetamido)acetic acid (58 mg) as a white solid.

¹H NMR (400 MHz, DMSO-d6): δ 9.05 (s, 1H), 8.66 (d, J=8 Hz, 1H), 8.58 (s, 1H), 7.95 (d, J=8 Hz, 1H), 7.69 (t, J=8 Hz, 1H), 7.47 (t, J=8 Hz, 1H), 7.36 (d, J=9 Hz, 2H), 6.91 (d, J=9 Hz, 2H), 5.20 (s, 2H), 4.37 (bs, 4H), 4.05 (s, 2H), 3.51 (bs, 4H); LC/MS(ESI-MS): [M+H]+=527.9.

### Example 133. 2-(2-(4-(4-hydroxyphenyl)piperazin-1-yl)-6-(indolin-1-yl)-9H-purin-9-yl)propane-1,3-diol

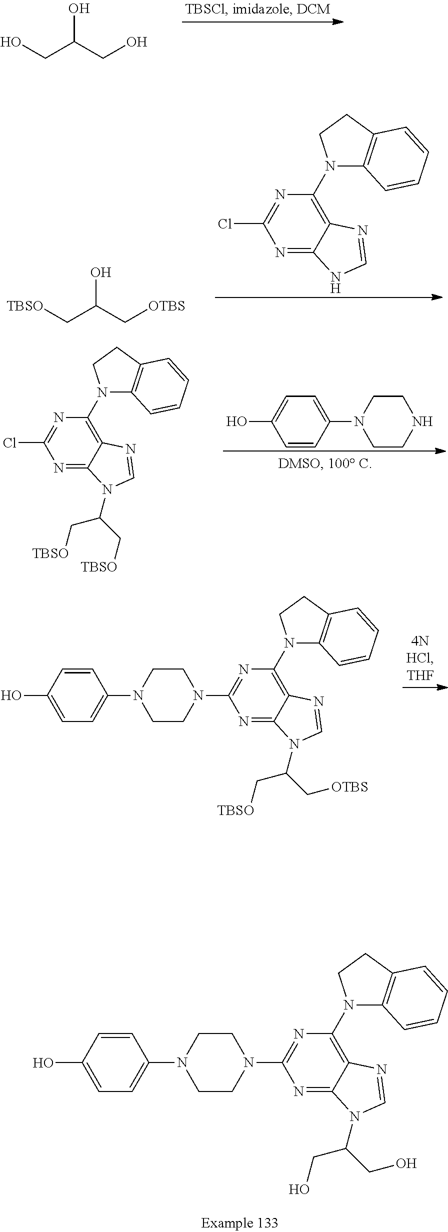

### Step 1:

To a solution of propane-1,2,3-triol (2 g, 21.72 mmol, 1.0 eq) in DCM (5 mL) at 25° C. was added imidazole (3.25 g, 47.78 mmol, 2.2 eq) and TBSCl (6.55 g, 43.44 mmol, 2.0 eq). The mixture was stirred for overnight under a nitrogen atmosphere before it was diluted with water (50 mL) and extracted with EtOAc (3×30 mL). The combined organic layer was washed with brine, dried over Na₂SO₄ then concentrated to give 2,2,3,3,9,9,10,10-octamethyl-4,8-dioxa-3,9-disilaundecan-6-ol (6.25 g, 89% yield) as a colorless oil.

### Step 2:

To a mixture of 2-chloro-6-(indolin-1-yl)-9H-purine (100 mg, 0.37 mmol, 1.0 eq) and 2,2,3,3,9,9,10,10-octamethyl-4,8-dioxa-3,9-disilaundecan-6-ol (176.33 mg, 0.55 mmol, 1.5 eq), PPh3 (145.2 mg, 0.55 mmol, 1.5 eq) in THF (2 mL) under a nitrogen atmosphere at 0° C. was added DIAD (111.22 mg, 0.55 mmol, 1.5 eq) dropwise. The reaction mixture was stirred from 0-25° C. overnight before it was concentrated under reduced pressure. The residue was purified by flash column chromatography on silica gel (petroleum ether:EtOAc=10:1) to give 2-chloro-6-(indolin-1-yl)-9-(2,2,3,3,9,9,10,10-octamethyl-4,8-dioxa-3,9-disilaundecan-6-yl)-9H-purine (200 mg, 95% yield) as a white solid.

### Step 3:

A mixture of 2-chloro-6-(indolin-1-yl)-9-(2,2,3,3,9,9,10,10-octamethyl-4,8-dioxa-3,9-disilaundecan-6-yl)-9H-purine (200 mg, 0.35 mmol, 1.0 eq) and 4-(piperazin-1-yl)phenol (200 mg, 0.35 mmol, 1.0 eq) in DMSO (2 mL) was stirred overnight at 100° C. under a nitrogen atmosphere. The reaction was allowed to cool and diluted with water (6 mL). The precipitate was collected by filtration and dried to provide crude 4-(4-(6-(indolin-1-yl)-9-(2,2,3,3,9,9,10,10-octamethyl-4,8-dioxa-3,9-disilaundecan-6-yl)-9H-purin-2-yl)piperazin-1-yl)phenol (350 mg) as a solid which was used in next step without further purification.

### Step 4:

To a solution of crude 4-(4-(6-(indolin-1-yl)-9-(2,2,3,3,9,9,10,10-octamethyl-4,8-dioxa-3,9-disilaundecan-6-yl)-9H-purin-2-yl)piperazin-1-yl)phenol (350 mg, 0.49 mmol, 1.0 eq) in MeoH (12 mL) at 25° C. was added 4N aq. HCl (4.88 mL, 19.55 mmol, 40 eq). The reaction was stirred for 2 hours. The white solid was collected by filtration and further purified by Prep-HPLC (Column: Waters SunFire Prep C18 OBD 5 um 19*150 mm Column; Mobile phase A: Acetonitrile; Mobile phase B: 0.1% TFA in Water; Flow rate: 20.0 ml/min; Detection UV: 210 nm) to give 2-(2-(4-(4-hydroxyphenyl)piperazin-1-yl)-6-(indolin-1-yl)-9H-purin-9-yl)propane-1,3-diol (79 mg, 33% yield) as a white solid.

¹H NMR (400 MHz, DMSO-d6): δ 10.11 (br, 1H), 8.29 (s, 1H), 8.18 (s, 1H), 7.68 (br, 2H), 7.30 (d, J=7.2 Hz, 1H), 7.22 (t, J=8.0 Hz, 1H), 6.99 (t, J=7.2 Hz, 1H), 6.90 (d, J=8.8 Hz, 2H), 4.74-4.53 (m, 5H), 3.92-3.82 (m, 6H), 3.81-3.65 (m, 4H), 3.25 (t, J=8.4 Hz, 2H); LC/MS(ESI-MS): [M+H]+=488.2.

### Example 134. 2-(2-(4-(4-hy-droxyphenyl)piperazin-1-yl)-6-(3-cyano-1H-indazol-1-yl)-9H-purin-9-yl)acetic acid

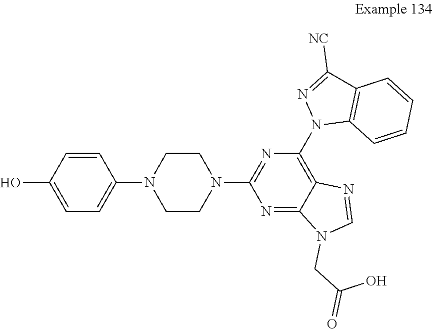 [SMILES: N#Cc1nn(-c2nc(N3CCN(c4ccc(O)cc4)CC3)nc3c2ncn3CC(=O)O)c2ccccc12]

The proceeding example was prepared according to Example 10 substituting ethyl 1H-indazole-3-carboxylate with 3-cyano-1H-indazole. The desired product 2-(2-(4-(4-hy-droxyphenyl)piperazin-1-yl)-6-(3-cyano-1H-indazol-1-yl)-9H-purin-9-yl)acetic acid was isolated as a white solid.

¹H NMR (400 MHz, DMSO-d6): δ 8.64 (d, J=8.6 Hz, 1H), 8.36 (d, J=6.9 Hz, 1H), 8.08 (d, J=8.1 Hz, 1H), 7.81 (t, J=7.7 Hz, 1H), 7.63 (t, J=7.6 Hz, 1H), 7.31-7.03 (m, 2H), 6.79 (d, J=8.4 Hz, 2H), 5.05 (s, 2H), 4.19-4.05 (m, 4H), 3.47-3.35 (m, 4H); LCMS(ESI-MS): [M+H]+=496.3.

### Example 135. 2-(6-(3-(1H-tetrazol-5-yl)-1H-indazol-1-yl)-2-(4-(4-hydroxyphenyl)piperazin-1-yl)-9H-purin-9-acetic acid

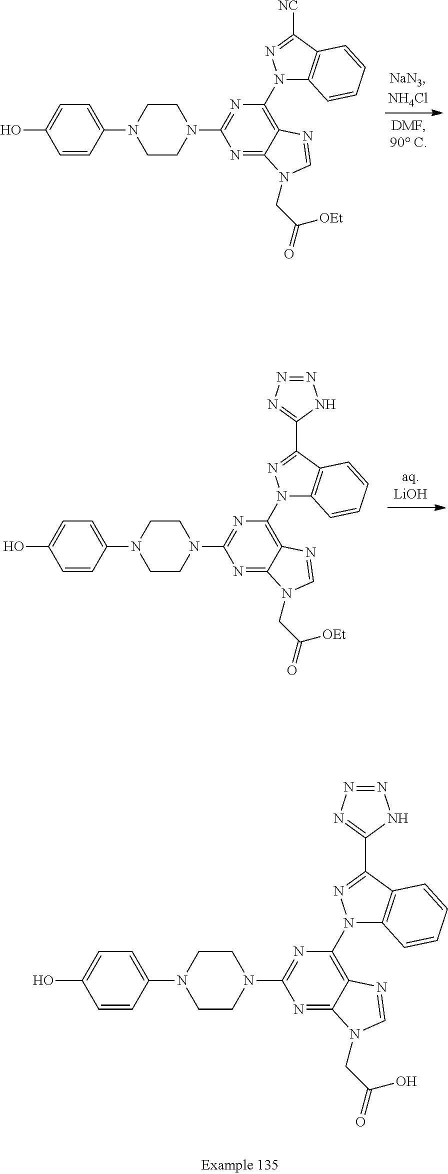 [SMILES: CCOC(=O)Cn1cnc2c(-n3nc(C#N)c4ccccc43)nc(N3CCN(c4ccc(O)cc4)CC3)nc21 ; CCOC(=O)Cn1cnc2c(-n3nc(-c4nnn[nH]4)c4ccccc43)nc(N3CCN(c4ccc(O)cc4)CC3)nc21 ; O=C(O)Cn1cnc2c(-n3nc(-c4nnn[nH]4)c4ccccc43)nc(N3CCN(c4ccc(O)cc4)CC3)nc21]

### Step 1:

To a solution of tert-butyl 2-(6-(3-cyano-1H-indazol-1-yl)-2-(4-(4-hydroxyphenyl)-piperazin-1-yl)-9H-purin-9-yl)acetate (55 mg, 0.1 mmol, 1.0 eq) in dry DMF (2.5 mL) was added NaN₃ (20 mg, 0.3 mmol, 3 eq) and NH4Cl (16 mg, 0.3 mmol, 3 eq). The resulting mixture was stirred at 90° C. overnight. The mixture was allowed to cool and partitioned between EtOAc (25 mL) and water (20 mL). The aqueous layer was extracted with EtOAc (2×25 mL). The combined organic phase was washed with brine (20 mL), dried over Na₂SO₄, filtered and concentrated. The crude product tert-butyl 2-(6-(3-(1H-tetrazol-5-yl)-1H-indazol-1-yl)-2-(4-(4-hydroxyphenyl)piperazin-1-yl)-9H-purin-9-yl)acetate (50 mg) was used for the next step directly without further purification.

### Step 2:

To a solution of tert-butyl 2-(6-(3-(1H-tetrazol-5-yl)-1H-indazol-1-yl)-2-(4-(4-hydroxy-phenyl)piperazin-1-yl)-9H-purin-9-yl)acetate (50 mg, crude) in DCM (2 mL) was added TFA (1 mL). The resulting mixture was stirred at room temperature overnight before it was concentrated. The crude product was purified by Prep-HPLC to 2-(6-(3-(1H-tetrazol-5-yl)-1H-indazol-1-yl)-2-(4-(4-hydroxyphenyl)piperazin-1-yl)-9H-purin-9-yl)acetic acid (8 mg, 16% yield) as an off-white solid.

¹H NMR (400 MHz, DMSO-d6): δ 8.61 (d, J=8.3 Hz, 1H), 8.48 (d, J=8.0 Hz, 1H), 8.40 (s, 1H), 7.78 (t, J=7.4 Hz, 1H), 7.61 (t, J=7.4 Hz, 1H), 7.08-6.96 (m, 2H), 6.74 (d, J=8.4 Hz, 2H), 5.06 (s, 3H), 4.14-3.99 (m, 5H), 3.33-3.19 (m, 4H); LC/MS(ESI-MS): [M+H]+=539.1.

### Example 136. 2-(2-(2-(4-(4-hydroxyphenyl)piperazin-1-yl)-6-(1H-indazol-1-yl)-9H-purin-9-yl)ethyl)isoindoline-1,3-dione

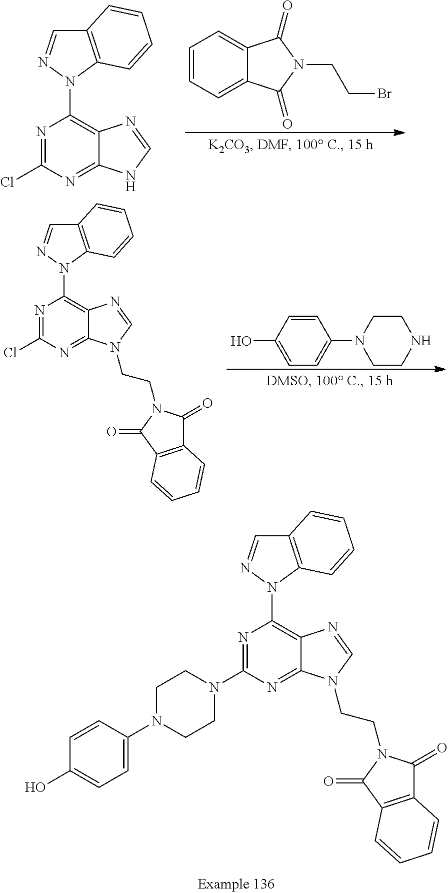

### Step 1:

A mixture of 2-chloro-6-(1H-indazol-1-yl)-9H-purine (0.25 g, 0.93 mmol, 1.0 eq) 2-(2-bromoethyl)isoindoline-1,3-dione (0.35 g, 1.39 mmol, 1.5 eq) and K₂CO₃ (0.38 g, 2.78 mmol, 3.0 eq) in DMSO (6 mL) under nitrogen was stirred at 100° C. overnight. The reaction mixture was allowed to cool and diluted with water (15 mL). The resulting solid was collected by filtration and dried to give 2-(2-(2-chloro-6-(1H-indazol-1-yl)-9H-purin-9-yl)ethyl)isoindoline-1,3-dione (0.33 g, 80% yield) as a solid.

### Step 2:

A mixture of 2-(2-(2-chloro-6-(1H-indazol-1-yl)-9H-purin-9-yl)ethyl)isoindoline-1,3-dione (0.33 g, 0.74 mmol, 1.0 eq) and 1-(4-hydroxyphenyl) piperazine (0.4 g, 2.23 mmol, 3.0 eq) in DMSO (6 mL) under nitrogen was stirred overnight at 100° C. The mixture was allowed to cool and diluted with water (15 mL) and the resulting precipitate was collected by filtration and dried to give 2-(2-(2-(4-(4-hydroxyphenyl)piperazin-1-yl)-6-(1H-indazol-1-yl)-9H-purin-9-yl)ethyl)isoindoline-1,3-dione (55 mg, 17% yield) as an off-white solid.

1H NMR (400 MHz, DMSO-d6): δ 8.89 (bs, 1H), 8.68 (s, 1H), 8.52 (d, J=8 Hz, 1H), 7.98 (d, J=8, 1H), 7.82-7.77 (m, 4H), 7.67 (t, J=8 Hz, 1H), 7.45 (t, J=8 Hz, 1H), 7.02 (bs, 2H), 6.75 (d, J=8 Hz, 2H), 4.52 (s, 2H), 4.09 (s, 2H), 3.83 (bs, 4H), 3.14 (bs, 4H); LC/MS(ESI-MS): [M+H]+=586.8.

### Example 137. 4-(4-(9-(2-aminoethyl)-6-(1H-indazol-1-yl)-9H-purin-2-yl)piperazin-1-yl)phenol

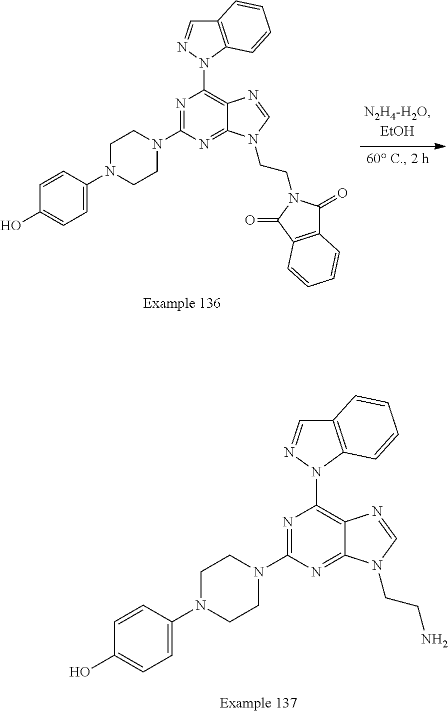 [SMILES: O=C1c2ccccc2C(=O)N1CCn1cnc2c(-n3ncc4ccccc43)nc(N3CCN(c4ccc(O)cc4)CC3)nc21 ; NCCn1cnc2c(-n3ncc4ccccc43)nc(N3CCN(c4ccc(O)cc4)CC3)nc21]

To a solution of 2-(2-(2-(4-(4-hydroxyphenyl)piperazin-1-yl)-6-(1H-indazol-1-yl)-9H-purin-9-yl)ethyl)isoindoline-1,3-dione (55 mg, 0.094 mmol, 1.0 eq) in EtOH (2 mL) was added hydrazine hydrate (1 mL). The mixture was stirred at 60° C. for 2 h before it was allowed to cool and diluted with water (5 mL). The resulting solid was collected by filtration and dried to give 4-(4-(9-(2-aminoethyl)-6-(1H-indazol-1-yl)-9H-purin-2-yl)piperazin-1-yl)phenol (30 mg, 71% yield) as a white solid.

1H NMR (400 MHz, DMSO-d6): δ 8.87 (s, 1H), 8.60 (d, J=8.5 Hz, 1H), 8.56 (s, 1H), 8.17 (s, 1H), 7.94 (d, J=7.9 Hz, 1H), 7.63 (t, J=7.7 Hz, 1H), 7.39 (t, J=7.5 Hz, 1H), 6.89 (d, J=8.8 Hz, 2H), 6.69 (d, J=8.8 Hz, 2H), 4.13 (t, J=6.1 Hz, 2H), 4.05-3.91 (m, 4H), 3.19-3.07 (s, 4H), 2.99 (t, J=6.1 Hz, 2H); LC/MS(ESI-MS): [M+H]+=455.9.

### Example 138. N-(2-(2-(4-(4-hydroxyphenyl)piperazin-1-yl)-6-(1H-indazol-1-yl)-9H-purin-9-yl)ethyl)acetamide

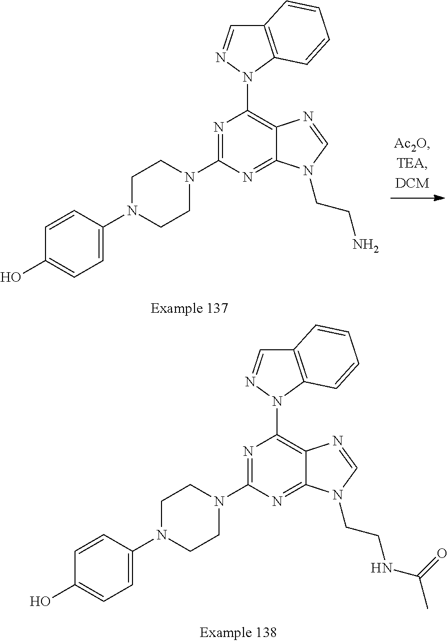 [SMILES: NCCn1cnc2c(-n3ncc4ccccc43)nc(N3CCN(c4ccc(O)cc4)CC3)nc21 ; CC(=O)NCCn1cnc2c(-n3ncc4ccccc43)nc(N3CCN(c4ccc(O)cc4)CC3)nc21]

To a solution of 4-(4-(9-(2-aminoethyl)-6-(1H-indazol-1-yl)-9H-purin-2-yl)piperazin-1-yl)phenol (20 mg, 0.044 mmol, 1.0 eq) in DMF (3 mL) at 0° C. was added Et3N (6.7 mg, 0.132 mmol, 3 eq) and Ac2O (2.3 mg, 0.044 mmol, 1.0 eq). The mixture was stirred at 25° C. overnight before it was diluted with water (10 mL). The resulting solid was collected by filtration and dried to give N-(2-(2-(4-(4-hydroxyphenyl)piperazin-1-yl)-6-(1H-indazol-1-yl)-9H-purin-9-yl)ethyl)acetamide (16 mg, 73% yield) as a white solid.

1H NMR (400 MHz, DMSO-d6): δ 8.88 (s, 1H), 8.61 (d, J=8.5 Hz, 1H), 8.57 (s, 1H), 8.11 (s, 1H), 8.03-7.88 (m, 2H), 7.63 (t, J=7.7 Hz, 1H), 7.39 (t, J=7.5 Hz, 1H), 6.89 (d, J=8.8 Hz, 2H), 6.69 (d, J=8.8 Hz, 2H), 4.22 (t, J=5.5 Hz, 2H), 4.07-3.91 (m, 4H), 3.50 (dd, J=11.1, 5.6 Hz, 2H), 3.21-3.07 (m, 4H), 1.76 (s, 3H); LC/MS(ESI-MS): [M+H]+=497.8.

### Example 139. N-(2-(2-(4-(4-hydroxyphenyl)piperazin-1-yl)-6-(1H-indazol-1-yl)-9H-purin-9-yl)ethyl)methanesulfonamide

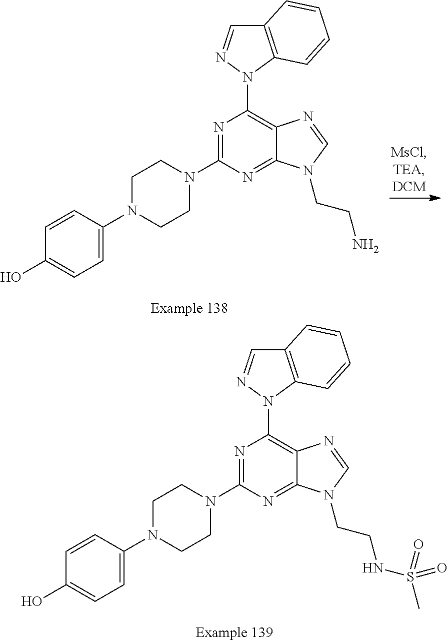 [SMILES: NCCn1cnc2c(-n3ncc4ccccc43)nc(N3CCN(c4ccc(O)cc4)CC3)nc21 ; CS(=O)(=O)NCCn1cnc2c(-n3ncc4ccccc43)nc(N3CCN(c4ccc(O)cc4)CC3)nc21]

To a solution of 4-(4-(9-(2-aminoethyl)-6-(1H-indazol-1-yl)-9H-purin-2-yl)piperazin-1-yl)phenol (20 mg, 0.044 mmol, 1.0 eq) in DMF (2 mL) at 0° C. was added Et3N (6.7 mg, 0.132 mmol, 3 eq) and MsCl (6 L, 0.066 mmol, 1.5 eq). The mixture was stirred at 25° C. overnight before it was diluted with water (8 mL). The resulting solid was collected by filtration and dried to give N-(2-(2-(4-(4-hydroxyphenyl)piperazin-1-yl)-6-(1H-indazol-1-yl)-9H-purin-9-yl)ethyl)methanesulfonamide (10 mg) as a white solid.

¹H NMR (400 MHz, DMSO-d6): δ 9.02 (bs, 1H), 8.66-8.62 (m, 2H), 7.98 (d, J=8 Hz, 1H), 7.66 (t, J=8 Hz, 1H), 7.44 (t, J=8 Hz, 1H), 7.32 (bs, 1H), 6.99 (bs, 2H), 6.72 (d, J=8 Hz, 2H), 4.33 (s, 2H), 4.07 (bs, 4H), 3.20 (bs, 4H), 2.91 (s, 3H); LC/MS(ESI-MS): [M+H]+=534.8.

### Example 140. 2-(2-(4-(1H-indazol-5-yl)piperazin-1-yl)-6-(1H-indazol-1-yl)-9H-purin-9-yl)acetic acid

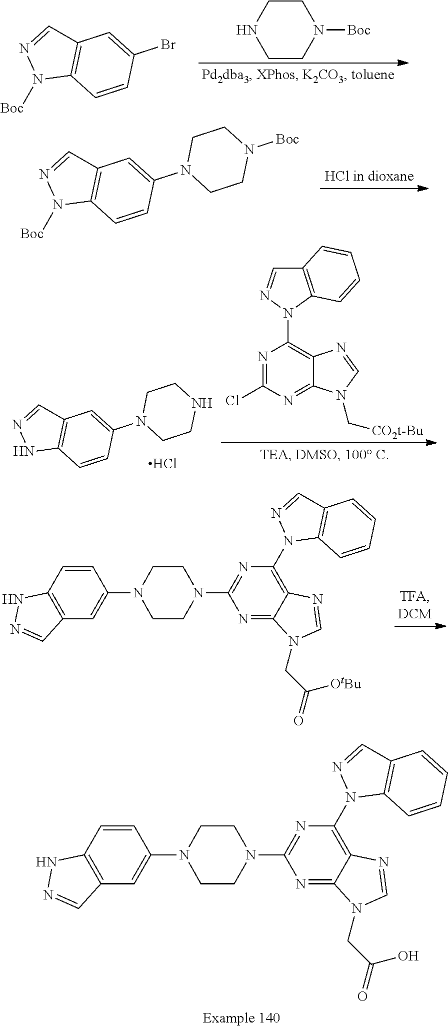

### Step 1:

A mixture of tert-butyl 5-bromo-1H-indazole-1-carboxylate (0.5 g, 1.69 mmol, 1.0 eq), K₂CO₃ (0.7 g, 5.03 mmol, 3.0 eq), 1-Boc-piperazine (0.47 g, 2.53 mmol, 1.5 eq), X-phos (0.16 g, 0.34 mmol, 0.2 eq) and Pd2(dba)3 (0.15 g, 0.17 mmol, 0.1 eq) in toluene (10 mL) under nitrogen atmosphere was stirred at 110° C. overnight. The reaction mixture was allowed to cool and diluted with water (30 mL) and extracted with EtOAc (3×20 mL). The combined organic layer was washed with brine, dried over Na₂SO₄ and concentrated. The crude residue was purified by flash column chromatography on silica gel (Petroleum ether:EtOAc=10:1) to give tert-butyl 5-(4-(tert-butoxycarbonyl)piperazin-1-yl)-1H-indazole-1-carboxylate (0.2 g, 28% yield) as an oil, yield.

### Step 2:

A mixture of tert-butyl 5-(4-(tert-butoxycarbonyl)piperazin-1-yl)-1H-indazole-1-carboxylate (0.2 g, 0.50 mmol, 1 eq) and 1M HCl solution (2 mL, 5.0 mmol, 2 M in dioxane, 10 eq) in DCM (5 mL) was stirred at 25° C. for 1 h. The reaction mixture was concentrated to give 5-(piperazin-1-yl)-1H-indazole hydrochloride (0.11 g, 88% yield) as a white solid.

### Step 3:

A mixture of 5-(piperazin-1-yl)-1H-indazole hydrochloride (0.11 g, 0.46 mmol, 2.5 eq), tert-butyl 2-(2-chloro-6-(1H-indazol-1-yl)-9H-purin-9-yl)acetate (77 mg, 0.18 mmol, 1 eq) and TEA (128 L, 0.92 mmol, 5 eq) in DMSO (3 mL) was stirred overnight at 100° C. The mixture was allowed to cool and diluted with water (10 mL) and the resulting precipitate was collected by filtration and dried to give tert-butyl 2-(2-(4-(1H-indazol-5-yl)piperazin-1-yl)-6-(1H-indazol-1-yl)-9H-purin-9-yl)acetate (0.14 g, 40% yield) as an off-white solid.

### Step 4:

A mixture of tert-butyl 2-(2-(4-(1H-indazol-5-yl)piperazin-1-yl)-6-(1H-indazol-1-yl)-9H-purin-9-yl)acetate (0.14 g, 0.25 mmol, 1 eq) and TFA (3 mL) in DCM (3 mL) was stirred for 0.5 h at room temperature before it was concentrated. The residue was purified by Prep-HPLC to give 2-(2-(4-(1H-indazol-5-yl)piperazin-1-yl)-6-(1H-indazol-1-yl)-9H-purin-9-yl)acetic acid (6 mg, 6% yield) as a white solid.

1H NMR (400 MHI-z, DMSO-d6): δ 9.74 (s, 1H), 8.37 (s, 1H), 7.96 (s, 1H), 7.87 (d, J=8.6 Hz, 1H), 7.79 (d, J=8.9 Hz, 1H), 7.50 (d, J=8.8 Hz, 1H), 7.42-7.23 (m, 3H), 7.19-7.07 (m, 1H), 5.04 (s, 2H), 4.14-4.02 (m, 4H), 3.34-3.21 (m, 4H); LC/MS(ESI-MS): [M+H]+=495.1.

### Example 141. 2-(2-(4-(5-hydroxypyridin-2-yl)piperazin-1-yl)-6-(1H-indazol-1-yl)-9H-purin-9-yl)-2-methylpropanoic acid

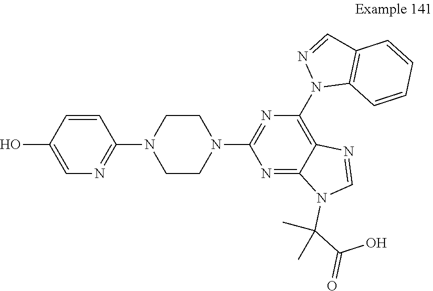 [SMILES: CC(C)(C(=O)O)n1cnc2c(-n3ncc4ccccc43)nc(N3CCN(c4ccc(O)cn4)CC3)nc21]

The proceeding example was prepared according to Example 21 substituting ethyl 2-bromoacetate with ethyl 2-bromo-2-methylpropanoate. The desired product 2-(2-(4-(5-hydroxypyridin-2-yl)piperazin-1-yl)-6-(1H-indazol-1-yl)-9H-purin-9-yl)-2-methylpropanoic acid was isolated as an off-white solid.

¹H NMR (400 MHz, DMSO-d6): δ 8.64-8.55 (m, 3H), 7.97 (d, J=8 Hz, 1H), 7.72 (s, 1H), 7.67 (t, J=8 Hz, 1H), 7.43 (t, J=8 Hz, 1H), 7.32 (d, J=8 Hz, 2H), 7.03 (d, J=8 Hz, 2H), 3.96 (bs, 4H), 3.95 (bs, 4H), 1.92 (s, 6H); LC/MS(ESI-MS): [M+H]+=500.0.

### Example 142. 2-(2-(4-(5-hydroxypyridin-2-yl)piperazin-1-yl)-6-(1H-indazol-1-yl)-9H-purin-9-yl)butanoic acid

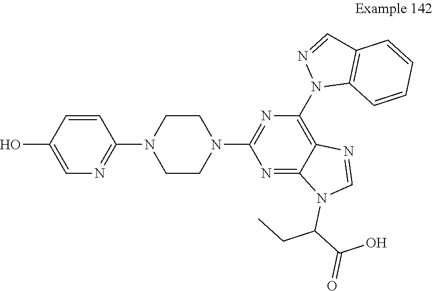 [SMILES: CCC(C(=O)O)n1cnc2c(-n3ncc4ccccc43)nc(N3CCN(c4ccc(O)cn4)CC3)nc21]

The proceeding example was prepared according to Example 21 substituting ethyl 2-bromoacetate with ethyl 2-bromobutanoate. The desired product 2-(2-(4-(5-hydroxypyridin-2-yl)piperazin-1-yl)-6-(1H-indazol-1-yl)-9H-purin-9-yl)butanoic acid was isolated as an off-white solid.

¹H NMR (400 MHz, DMSO-d6): δ 8.64-8.62 (m, 3H), 7.97 (d, J=8 Hz, 1H), 7.69-7.65 (m, 2H), 7.44 (t, J=8 Hz, 2H), 7.12 (bs, 1H), 5.23 (s, 1H), 4.01 (bs, 4H), 3.63 (bs, 4H), 2.40-2.31 (m, 2H), 0.88 (t, J=7 Hz, 3H); LC/MS(ESI-MS): [M+H]+=500.9.

### Example 143. 2-(6-(4-((dimethylamino)methyl)-1H-indazol-1-yl)-2-(4-(4-hydroxyphenyl)piperazin-1-yl)-9H-purin-9-yl)acetic acid

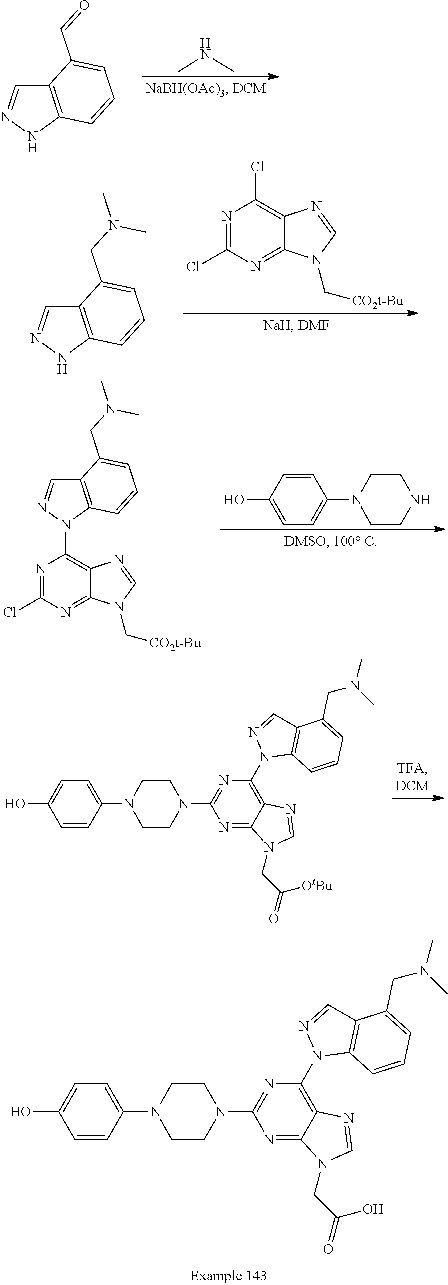

### Step 1:

A mixture of 1H-indazole-4-carbaldehyde (1 g, 6.84 mmol, 1.0 eq), 2M dimethylamine in THF (17.1 mL, 34.21 mmol, 5.0 eq) and acetic acid (5 drop) in DCM (10 mL) was stirred at room temperature for 1.5 h. To the mixture was added NaBH(OAc)₃ (2.18 g, 10.26 mmol, 1.5 eq) and the mixture was stirred at room temperature overnight. The mixture was diluted with water and then adjusted to pH 10 with saturated aqueous Na₂CO₃ solution (5 mL). The resulting solid was filtered off, and the filtrate was extracted with DCM (3×15 mL). The combined organic layers was dried (Na₂SO₄) and concentrated to give crude 1-(1H-indazol-4-yl)-N,N-dimethylmethanamine (600 mg, 51% yield) as a solid.

### Step 2:

To a solution of crude 1-(1H-indazol-4-yl)-N,N-dimethylmethanamine (250 mg, 1.43 mmol, 1.0 eq) in DMF (3 mL) stirring at 0° C. under N₂ was add NaH (68. 5 mg, 1.71 mmol, 1.2 eq). The reaction mixture was stirred at 0° C. for 30 min and followed by addition of tert-butyl 2-(2,6-dichloro-9H-purin-9-yl)acetate (432 mg, 1.43 mmol, 1.0 eq). The mixture was stirred at room temperature for 1 h before it was poured into water (20 mL). The resulting precipitate was collected by filtration and dried in vacuo. The crude product was purified by flash column chromatography on silica gel (DCM:MeOH=30:1) to give tert-butyl 2-(2-chloro-6-(4-((dimethylamino)methyl)-1H-indazol-1-yl)-9H-purin-9-yl)acetate (75 mg, 12% yield) as a light yellow solid.

### Step 3:

To a solution of tert-butyl 2-(2-chloro-6-(4-((dimethylamino)methyl)-1H-indazol-1-yl)-9H-purin-9-yl)acetate (60 mg, 0.14 mmol, 1.0 eq) in DMSO (2 mL) was added 4-(piperazin-1-yl)phenol (60.5 mg, 0.34 mmol, 2.5 eq) and the mixture was stirred at 100° C. overnight under nitrogen. The reaction mixture was cooled to room temperature and diluted with water (20 mL). The resulting precipitate was collected by filtration. The filtrate was further was extracted with a mixture of DCM and MeOH (10:1). The organic layer was dried and concentrated to provide a crude solid. The combined crude solid was dried to give tert-butyl 2-(6-(4-((dimethylamino)methyl)-1H-indazol-1-yl)-2-(4-(4-hydroxyphenyl)piperazin-1-yl)-9H-purin-9-yl)acetate (150 mg) as a light yellow solid.

### Step 4:

To a solution of tert-butyl 2-(6-(4-((dimethylamino)methyl)-1H-indazol-1-yl)-2-(4-(4-hydroxyphenyl)piperazin-1-yl)-9H-purin-9-yl)acetate (150 mg, 0.257 mmol, 1.0 eq) in DCM (2 mL) was added TFA (2 mL). The mixture was stirred at room temperature for 4 h before it was concentrated. The crude product was purified by prep-HPLC to give 2-(6-(4-((dimethyl-amino)methyl)-1H-indazol-1-yl)-2-(4-(4-hydroxyphenyl)piperazin-1-yl)-9H-purin-9-yl)acetic acid (25 mg, 18% yield) as a white solid.

¹H NMR (400 MHz, DMSO-d6): δ 9.98 (br s, 1H), 8.95 (s, 1H), 8.68 (d, J=8.5 Hz, 1H), 8.34 (s, 1H), 7.80-7.66 (m, 1H), 7.56 (d, J=7.2 Hz, 1H), 7.06 (s, 2H), 6.75 (d, J=8.8 Hz, 2H), 5.03 (s, 2H), 4.74 (s, 2H), 4.18-3.95 (m, 4H), 3.37-3.21 (m, 4H), 2.84 (s, 5H); LC/MS(ESI-MS): [M+H]+=528.0.

### Example 144. 2-(6-(4-((dimethylamino)methyl)-1H-indazol-1-yl)-2-(4-(5-hydroxypyridin-2-yl)piperazin-1-yl)-9H-purin-9-yl)acetic acid

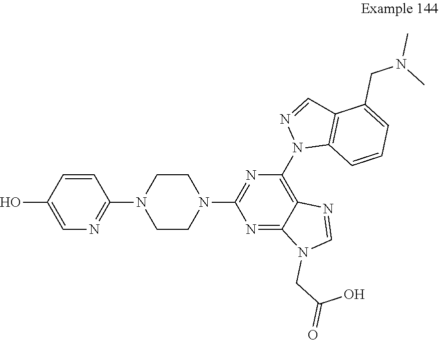 [SMILES: CN(C)Cc1cccc2c1cnn2-c1nc(N2CCN(c3ccc(O)cn3)CC2)nc2c1ncn2CC(=O)O]

The proceeding example was prepared according to Example 143 substituting 4-(piperazin-1-yl)phenol with 6-(piperazin-1-yl)pyridin-3-ol. The desired product 2-(6-(4-((dimethylamino)methyl)-1H-indazol-1-yl)-2-(4-(5-hydroxypyridin-2-yl)piperazin-1-yl)-9H-purin-9-yl)acetic acid was isolated as an off-white solid.

¹H NMR (400 MHz, DMSO-d6): δ 10.03 (br s, 1H), 8.95 (s, 1H), 8.70 (d, J=8.5 Hz, 1H), 8.37 (s, 1H), 7.81-7.68 (m, 2H), 7.57 (d, J=7.2 Hz, 1H), 7.40 (d, J=8.4 Hz, 1H), 7.09 (d, J=9.2 Hz, 1H), 5.04 (s, 2H), 4.75 (s, 2H), 4.08-3.92 (m, 4H), 3.71-3.56 (m, 4H), 2.85 (s, 6H); LC/MS(ESI-MS): [M+H]+=529.0.

### Example 145. 2-(2-(4-(5-hydroxypyridin-2-yl)piperazin-1-yl)-6-(1H-indazol-1-yl)-9H-purin-9-yl)propanoic acid

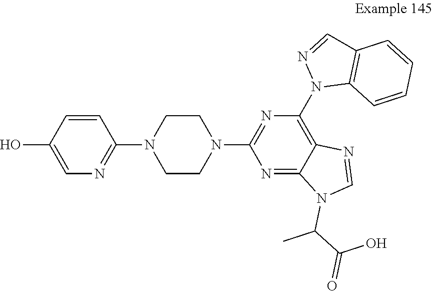 [SMILES: CC(C(=O)O)n1cnc2c(-n3ncc4ccccc43)nc(N3CCN(c4ccc(O)cn4)CC3)nc21]

The proceeding example was prepared according to Example 21 substituting ethyl 2-(2-chloro-6-(1H-indazol-1-yl)-9H-purin-9-yl)acetate with ethyl 2-(2-chloro-6-(1H-indazol-1-yl)-9H-purin-9-yl)propanoate. The desired product 2-(2-(4-(5-hydroxypyridin-2-yl)pipera-zin-1-yl)-6-(1H-indazol-1-yl)-9H-purin-9-yl)propanoic acid was isolated as an off-white solid.

¹H NMR (400 MHz, DMSO-d6): δ 8.79 (s, 1H), 8.67-8.62 (m, 2H), 7.99-7.97 (m, 1H), 7.69-7.66 (m, 2H), 7.47-7.43 (m, 2H), 7.16 (d, J=9.2 Hz, 1H), 5.47-5.41 (m, 1H), 4.10-3.95 (m, 4H), 3.69-3.54 (m, 4H), 1.87 (d, J=7.2 Hz, 3H); LC/MS(ESI-MS): [M+H]+=486.0.

### Example 146. 4-(2-(4-(4-hydroxyphenyl)piperazin-1-yl)-6-(1H-indazol-1-yl)-9H-purin-9-yl)butanoic acid

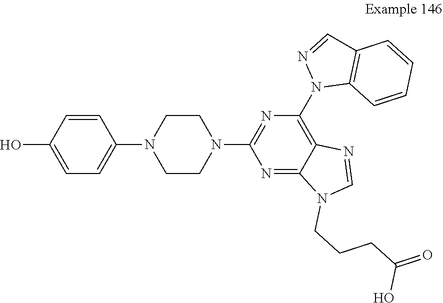 [SMILES: O=C(O)CCCn1cnc2c(-n3ncc4ccccc43)nc(N3CCN(c4ccc(O)cc4)CC3)nc21]

The proceeding example was prepared according to Example 23 substituting methyl 3-bromopropanoate with ethyl 4-bromobutanoate. The desired product 4-(2-(4-(4-hydroxy-phenyl)piperazin-1-yl)-6-(1H-indazol-1-yl)-9H-purin-9-yl)butanoic acid was isolated as an off-white solid.

¹H NMR (400 MHz, DMSO-d6): δ 9.01 (br, 1H), 8.72 (s, 1H), 8.63 (d, J=8.4 Hz, 1H), 8.00 (d, J=7.6 Hz, 1H), 7.73-7.69 (m, 1H), 7.47 (d, J=7.6 Hz, 1H), 7.11-6.99 (m, 2H), 6.75 (d, J=8.8 Hz, 2H), 4.31 (t, J=6.8 Hz, 1H), 4.15-4.01 (m, 4H), 3.33-3.24 (m, 4H), 2.37-2.32 (m, 2H), 2.17-2.08 (m, 2H); LC/MS(ESI-MS): [M+H]+=499.2.

### Example 147. 2-(6-(3-allyl-1H-indazol-1-yl)-2-(4-(4-hydroxyphenyl)piperazin-1-yl)-9H-purin-9-yl)acetic acid

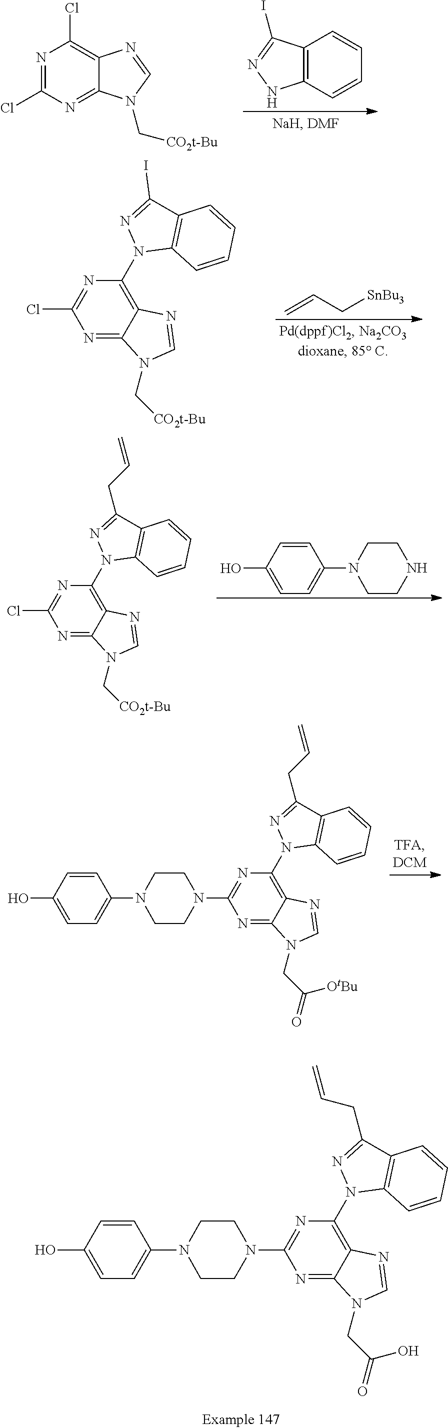

### Step 1:

To a solution of 3-iodo-1H-indazole (1.6 g, 6.6 mmol, 1.0 eq) in DMF (15 mL) at 0° C. under N₂ atmosphere was added NaH (320 mg, 7.9 mmol, 1.2 eq). The reaction mixture was stirred for 30 mins at 0° C. and followed by addition of tert-butyl 2-(2,6-dichloro-9H-purin-9-yl)acetate (2 g, 6.6 mmol, 1.0 eq) was added. The reaction mixture was stirred at 25° C. overnight before it was diluted with water (10 mL). The resulting precipitate was collected by filtration and dried to give tert-butyl 2-(2-chloro-6-(3-iodo-1H-indazol-1-yl)-9H-purin-9-yl)acetate (2.4 g, 72% yield) as a white solid Step 2:

A mixture of tert-butyl 2-(2-chloro-6-(3-iodo-1H-indazol-1-yl)-9H-purin-9-yl)acetate (500 mg, 0.98 mmol, 1.0 eq), Allyltributyltin (485 mg, 1.46 mmol, 1.0 eq), Pd(dppf)Cl2 (80 mg, 0.1 mmol, 0.1 eq), Na₂CO₃ (310 mg, 2.94 mmol, 3.0 eq) in a mixture of water (2 mL) and dioxane (10 mL) was stirred at 100° C. overnight. The reaction mixture was allowed to cool and concentrated to give the crude product. The crude product was purified with flash column chromatography on silica gel (petroleum ether:EtOAc=1:1) to give tert-butyl 2-(6-(3-allyl-1H-indazol-1-yl)-2-chloro-9H-purin-9-yl)acetate (200 mg, 48% yield) as a white solid.

### Step 3:

A mixture of tert-butyl 2-(6-(3-allyl-1H-indazol-1-yl)-2-chloro-9H-purin-9-yl)acetate (200 mg, 0.47 mmol, 1.0 eq) in DMSO (4 mL) and 4-(piperazin-1-yl) phenol (250 mg, 1.41 mmol, 3 eq) under N₂ atmosphere was stirred at 100° C. overnight. The mixture was allowed to cooled and diluted with water (10 mL). The resulting precipitate was collected and dried to give tert-butyl 2-(6-(3-allyl-1H-indazol-1-yl)-2-(4-(4-hydroxyphenyl)piperazin-1-yl)-9H-purin-9-yl)acetate (200 mg, 75% yield) as a brown solid.

### Step 4:

A mixture of tert-butyl 2-(6-(3-allyl-1H-indazol-1-yl)-2-(4-(4-hydroxyphenyl)piperazin-1-yl)-9H-purin-9-yl)acetate (200 mg, 0.35 mmol, 1.0 eq) and TFA (1 mL) in DCM (1 mL) was stirred at 25° C. for 4 h. The reaction mixture was concentrated under reduced pressure. The crude product was then purified by prep-HPLC to give (60 mg, 33% yield) as an off-white solid.

¹H NMR (400 MHz, DMSO-d6): δ 7.83-7.72 (m, 3H), 7.55-7.45 (m, 4H), 6.96-6.93 (m, 2H), 6.17-6.10 (m, 1H), 5.21-5.15 (m, 2H), 5.09-5.04 (m, 2H), 4.96 (s, 2H), 4.19-4.03 (m, 4H), 3.66-3.64 (m, 4H); LC/MS(ESI-MS): [M+H]+=511.9.

### Example 148. 4-(2-(4-(4-hydroxyphenyl)piperazin-1-yl)-6-(1H-indazol-1-yl)-9H-purin-9-yl)propanoic acid

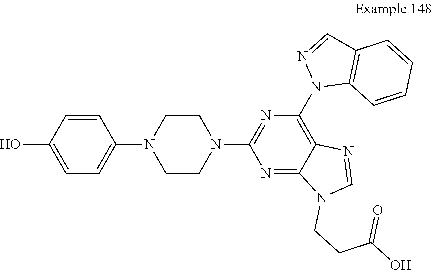 [SMILES: O=C(O)CCn1cnc2c(-n3ncc4ccccc43)nc(N3CCN(c4ccc(O)cc4)CC3)nc21]

The proceeding example was prepared according to Example 23 by hydrolysis of the corresponding methyl ester with aq. LiOH solution in THF to yield the desired product. The desired product 4-(2-(4-(4-hydroxy-phenyl)piperazin-1-yl)-6-(1H-indazol-1-yl)-9H-purin-9-yl)butanoic acid was isolated as an off-white solid.

¹H NMR (400 MHz, DMSO-d6): δ 8.95 (br, 1H), 8.67-8.58 (m, 2H), 7.98 (bs, 1H), 7.69 (bs, 1H), 7.46 (bs, 1H), 7.36-7.19 (d, J=6 Hz, 2H), 6.81-6.78 (d, J=8 Hz, 2H), 4.45 (bs, 2H), 4.14 (bs, 4H), 3.34 (bs, 4H), 3.00 (bs, 2H); LC/MS(ESI-MS). [M+H]+=485.1.

### Example 149. 5-(2-(4-(5-hydroxypyridin-2-yl)piperazin-1-yl)-6-(1H-indazol-1-yl)-9H-purin-9-yl)pentanoic acid

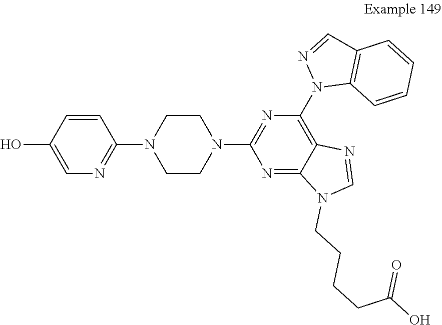 [SMILES: O=C(O)CCCCn1cnc2c(-n3ncc4ccccc43)nc(N3CCN(c4ccc(O)cn4)CC3)nc21]

The proceeding example was prepared according to Example 21 by substituting ethyl 2-bromoacetate with methyl 5-bromovalerate. The desired product 5-(2-(4-(5-hydroxypyridin-2-yl)piperazin-1-yl)-6-(1H-indazol-1-yl)-9H-purin-9-yl)pentanoic acid was isolated as a white solid.

¹H NMR (400 MHz, DMSO-d6): δ 9.04 (s, 1H), 8.72 (s, 1H), 8.65 (d, J=8.4 Hz, 1H), 8.00 (d, J=8.0 Hz, 1H), 7.73-7.69 (m, 2H), 7.49-7.45 (m, 1H), 7.42-7.39 (m, 1H), 7.14-7.09 (m, 1H), 4.28 (t, J=6.8 Hz, 2H), 4.07-4.04 (m, 4H), 3.70-3.58 (m, 4H), 2.32 (t, J=7.6 Hz, 2H), 1.97-1.90 (m, 2H), 1.58-1.50 (m, 2H); LC/MS(ESI-MS): [M+H]+=514.2.

### Example 150. 5-(2-(4-(4-hydroxyphenyl)piperazin-1-yl)-6-(1H-indazol-1-yl)-9H-purin-9-yl)pentanoic acid

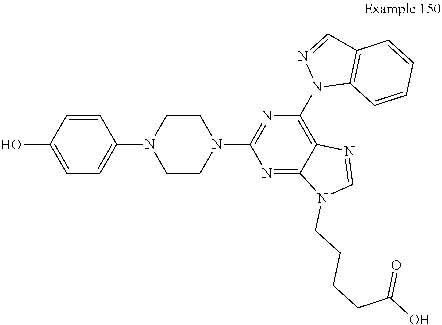 [SMILES: O=C(O)CCCCn1cnc2c(-n3ncc4ccccc43)nc(N3CCN(c4ccc(O)cc4)CC3)nc21]

The proceeding example was prepared according to Example 21 by substituting ethyl 2-bromoacetate with methyl 5-bromovalerate, substituting 6-(piperazin-1-yl)pyridin-3-ol hydrochloride with 4-(piperazin-1-yl)phenol. The desired product 5-(2-(4-(4-hydroxyphenyl) piperazin-1-yl)-6-(1H-indazol-1-yl)-9H-purin-9-yl)pentanoic acid was isolated as a white solid.

¹H NMR (400 MHz, DMSO-d6): δ 8.62-8.57 (m, 2H), 8.22 (s, 1H), 7.95 (d, J=8.0 Hz, 1H), 7.63 (t, J=7.6 Hz, 1H), 7.39 (t, J=7.6 Hz, 1H), 6.89 (d, J=8.8 Hz, 2H), 6.70 (d, J=8.8 Hz, 2H), 4.18 (t, J=6.4 Hz, 2H), 4.01-3.91 (m, 4H), 3.16-3.03 (m, 4H), 2.31 (t, J=6.8 Hz, 2H), 1.90-1.85 (m, 2H), 1.54-1.48 (m, 2H); LC/MS(ESI-MS): [M+H]+=513.3.

### Example 151. 3-cyclopropyl-2-(2-(4-(5-hydroxypyridin-2-yl)piperazin-1-yl)-6-(1H-indazol-1-yl)-9H-purin-9-yl)propanoic acid

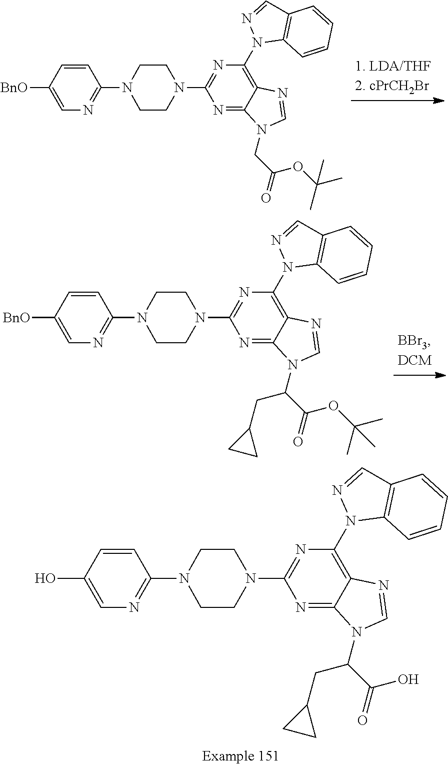

### Step 1:

To a solution of tert-butyl 2-(2-(4-(5-(benzyloxy)pyridin-2-yl)piperazin-1-yl)-6-(1H-indazol-1-yl)-9H-purin-9-yl)acetate (300 mg, 0.48 mmol, 1.0 eq) in THF (5 mL) at −78° C. under N₂, LDA (0.73 ml, 1.46 mmol, 3.0 eq) was added and allowed to stirred for 10 minutes. (Bromomethyl)cyclopropane (196 mg, 1.46 mmol, 3.0 eq) was added. The reaction mixture was stirred at room temperature for 12 hours. The reaction was quenched with water (10 mL) at 0° C. The reaction mixture was extracted with ethyl acetate (30 mL×3). The combined organic layers were washed with water (10 mL), brine (10 mL), dried with MgSO₄, filtered and concentrated. The residue was purified with silica-gel chromatography (PE. EA=3:1) to give tert-butyl 2-(2-(4-(5-(benzyloxy)pyridin-2-yl)piperazin-1-yl)-6-(1H-indazol-1-yl)-9H-purin-9-yl)-3-cyclopropylpropanoate (50 mg, 16% yield) as a yellow solid.

### Step 2

To a solution of tert-butyl 2-(2-(4-(5-(benzyloxy)pyridin-2-yl)piperazin-1-yl)-6-(1H-indazol-1-yl)-9H-purin-9-yl)-3-cyclopropylpropanoate (50 mg, 0.07 mmol, 1.0 eq) in dichloromethane (3 mL) at 0° C. under N₂, boron tibromide (93 mg, 0.37 mmol, 5 eq) was added. The mixture was stirred at 25° C. for 2 hours. The mixture was quenched with water (10 mL) at 0° C. The reaction mixture was extracted with ethyl acetate (30 mL×3). The combined organic layers were washed with brine (10 mL), dried with MgSO4, filtered and concentrated. The residue was purified by preparative HPLC to give 3-cyclopropyl-2-(2-(4-(5-hydroxypyridin-2-yl)piperazin-1-yl)-6-(1H-indazol-1-yl)-9H-purin-9-yl)propanoic acid (10 mg, 27% yield) as a white solid.

¹HNMR (400 MHz, CD3OD): δ 8.17-8.11 (m, 1H), 7.91 (d, J=8.4 Hz, 1H), 7.76-7.67 (m, 3H), 7.44 (s, 1H), 7.37-7.30 (m, 2H), 5.39-5.30 (m, 1H), 3.99-3.97 (m, 4H), 3.67-3.60 (m, 4H), 2.37-2.28 (m, 1H), 2.13-2.04 (m, 1H), 0.65-0.55 (m, 1H), 0.49-0.31 (m, 2H), 0.17-0.06 (m, 1H); LC/MS(ESI-MS): [M+H]+=526.2.

### Example 152. 4-(4-(9-(2-hydroxyethyl)-6-(3-(hydroxymethyl)-1H-indazol-1-yl)-9H-purin-2-yl)piperazin-1-yl)phenol

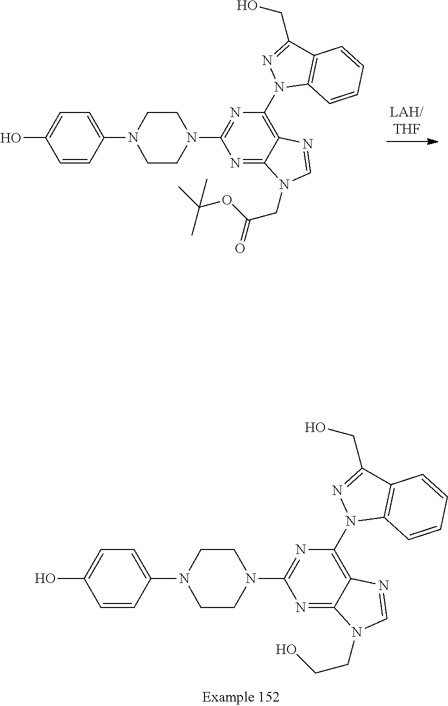 [SMILES: CC(C)(C)OC(=O)Cn1cnc2c(-n3nc(CO)c4ccccc43)nc(N3CCN(c4ccc(O)cc4)CC3)nc21 ; OCCn1cnc2c(-n3nc(CO)c4ccccc43)nc(N3CCN(c4ccc(O)cc4)CC3)nc21]

To a solution of tert-butyl 2-(6-(3-(hydroxymethyl)-1H-indazol-1-yl)-2-(4-(4-hydroxyphenyl)piperazin-1-yl)-9H-purin-9-yl)acetate (100 mg, 0.18 mmol, 1.0 eq) in THF (3 mL) at 0° C., lithium aluminum hydride (34 mg, 0.90 mmol, 5.0 eq) was added. The mixture was stirred at room temperature for 12 hours. The reaction mixture was quenched with water (10 mL) to obtain a solid. The solid was dissolved in methanol (2 mL) and was purified by preparative HPLC to give 4-(4-(9-(2-hydroxyethyl)-6-(3-(hydroxymethyl)-1H-indazol-1-yl)-9H-purin-2-yl)piperazin-1-yl)phenol (2.6 mg, 3% yield) as a yellow solid.

1HNMR (400 MHz, DMSO-d6): δ 8.58 (d, J=8.4 Hz, 1H), 8.13 (s, 1H), 8.03 (d, J=8.0 Hz, 1H), 7.62 (t, J=7.6 Hz, 1H), 7.39 (t, J=7.6 Hz, 1H), 6.89 (d, J=8.8 Hz, 2H), 6.69 (d, J=9.2 Hz, 2H), 4.92 (s, 2H), 4.21 (t, J=5.6 Hz, 2H), 4.01-3.92 (m, 4H), 3.80 (t, J=5.6 Hz, 2H), 3.17-3.09 (m, 4H); LC/MS(ESI-MS): [M+H]+=487.2.

### Example 153. 4-(4-(9-(2-hydroxyethyl)-6-(1H-indazol-1-yl)-9H-purin-2-yl)piperazin-1-yl)-2-methylphenol

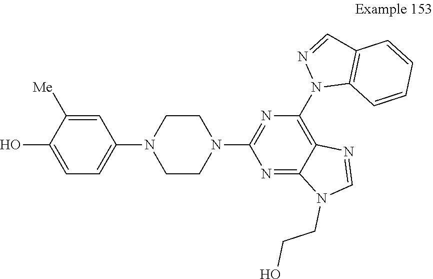 [SMILES: Cc1cc(N2CCN(c3nc(-n4ncc5ccccc54)c4ncn(CCO)c4n3)CC2)ccc1O]

The proceeding example was prepared according to Example 3 by substituting 4-(piperazin-1-yl)phenol with 2-methyl-4-(piperazin-1-yl)phenol. The desired product 4-(4-(9-(2-hydroxyethyl)-6-(1H-indazol-1-yl)-9H-purin-2-yl)piperazin-1-yl)-2-methylphenol was isolated as a white solid.

¹HNMR (400 MHz, DMSO-d6): δ 8.96 (br, 2H), 8.66-8.58 (m, 3H), 7.98 (d, J=8.0 Hz, 1H), 7.67 (t, J=7.6 Hz, 1H), 7.44 (t, J=7.6 Hz, 1H), 7.01-6.83 (m, 2H), 6.73 (d, J=8.4 Hz, 1H), 4.29-4.26 (m, 3H), 4.17-3.95 (m, 4H), 3.84 (t, J=6.4 Hz, 2H), 3.29-3.18 (m, 4H), 2.12 (s, 3H); LC/MS(ESI-MS): [M+H]+=471.2.

### Example 154. 2-(2-(4-(5-(hydroxymethyl)pyridin-2-yl)piperazin-1-yl)-6-(1H-indazol-1-yl)-9H-purin-9-yl)ethan-1-ol 947

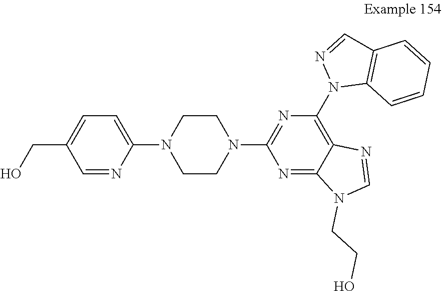 [SMILES: OCCn1cnc2c(-n3ncc4ccccc43)nc(N3CCN(c4ccc(CO)cn4)CC3)nc21]

The proceeding example was prepared according to Example 3 by substituting 4-(piperazin-1-yl)phenol with (6-(piperazin-1-yl)pyridin-3-yl)methanol. The desired product 2-(2-(4-(5-(hydroxymethyl)pyridin-2-yl)piperazin-1-yl)-6-(1H-indazol-1-yl)-9H-purin-9-yl)ethan-1-ol was isolated as a white solid.

¹H NMR (400 MHz, DMSO-d6): δ 8.67-8.63 (m, 3H), 8.04 (s, 1H), 7.98 (d, J=8.0 Hz, 1H), 7.69 (t, J=8.0 Hz, 2H), 7.47-7.43 (m, 1H), 7.16-7.06 (m, 1H), 4.41 (s, 2H), 4.27-4.23 (m, 2H), 4.16-4.01 (m, 4H), 3.98-3.72 (m, 6H); LC/MS(ESI-MS): [M+H]+=472.2.

### Example 155. 1-(6-amino-9-(pyridin-4-yl)-9H-purin-2-yl)-4-(4-hydroxyphenyl)piperidin-4-ol

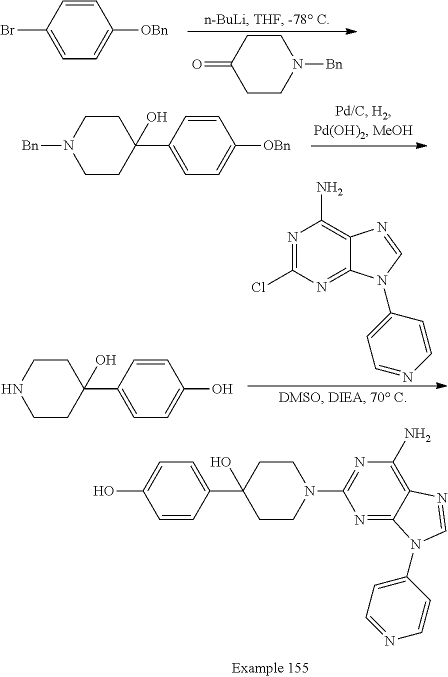

### Step 1

To a solution of 1-(benzyloxy)-4-bromobenzene (10.0 g, 38.00 mmol, 1.0 eq) in THF (80 mL) under nitrogen at −78° C., n-BuLi (26 ml, 60.81 mmol, 1.6 eq) was added and the reaction was stirred at −78° C. for 30 minutes, 1-benzylpiperidin-4-one (8.0 g, 42 mmol, 1.1 eq) was added and the reaction was stirred at the same temperature for 1.5 hour. Water (10 mL) was added to quench the reaction at low temperature. The resulting solution was warmed to room temperature. The mixture was extracted with ethyl acetate (150 mL×3). The combined organic layers were washed with brine, dried over Na₂SO₄, filtered and concentrated under vacuum to give 1-benzyl-4-(4-(benzyloxy)phenyl)piperidin-4-ol (11 g, 77% yield) as a white solid.

### Step 2

To a solution of 1-benzyl-4-(4-(benzyloxy)phenyl)piperidin-4-ol (11 g, 29 mmol, 1.0 eq) in MeOH (50 mL), Pd/C (2.2 g, 20%) and Pd(OH)2/C (2.2 g, 10%) were added. The reaction was stirred under a balloon pressure of hydrogen gas at room temperature for 15 hours. Dichloromethane (50 mL) and methanol (50 mL) was added to dilute the reaction mixture. Filtered off the solid and evaporated the solvent in the filtrate under vacuum to give 4-(4-hydroxyphenyl)piperidin-4-ol (2.7 g, 47% yield) as a gray solid.

### Step 3

To a solution of 4-(4-hydroxyphenyl)piperidin-4-ol (588 mg, 3.04 mmol, 2.5 eq) in DMSO (10 mL), 2-chloro-9-(pyridin-4-yl)-9H-purin-6-amine (300 mg, 1.22 mmol, 1.0 eq), DIEA (786 mg, 6.08 mmol, 5.0 eq) were added. The mixture was heated to 70° C. for 12 hours. The reaction mixture was cooled and water (10 mL) was added to get a precipitate. Collected the solid by filtration and purified the crude product silica gel column chromatography (DCM:MeOH=10:1, v/v) to give 1-(6-amino-9-(pyridin-4-yl)-9H-purin-2-yl)-4-(4-hydroxyphenyl)piperidin-4-ol (300 mg, 61%) as a white solid. ¹HNMR (400 MHz, DMSO-d6): δ 9.17 (s, 1H), 8.72-8.70 (m, 2H), 8.50 (s, 1H), 7.27 (d, J=8.4 Hz, 2H), 7.01 (s, 2H), 6.68 (d, J=8.4 Hz, 2H), 4.86 (s, 1H), 4.58-4.55 (m, 2H), 3.28-3.26 (m, 2H), 1.86-1.80 (m, 2H), 1.67-1.64 (m, 2H); LC/MS(ESI-MS): [M+H]+=404.1.

### Example 156. 4-(1-(6-amino-9-(pyridin-4-yl)-9H-purin-2-yl)-1,2,3,6-tetrahydropyridin-4-yl)phenol

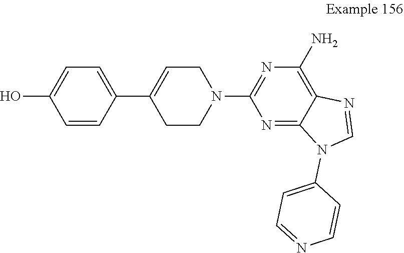 [SMILES: Nc1nc(N2CC=C(c3ccc(O)cc3)CC2)nc2c1ncn2-c1ccncc1]

The proceeding example was prepared according to Example 155 without adding DIEA and heated the reaction at 100° C. instead of 70° C. in the last synthetic step. The desired product 4-(1-(6-amino-9-(pyridin-4-yl)-9H-purin-2-yl)-1,2,3,6-tetrahydropyridin-4-yl)phenol was isolated as a white solid.

¹H NMR (400 MHz, DMSO-d6): δ 9.41 (s, 1H), 8.74-8.72 (m, 2H), 8.53 (s, 1H), 8.23-8.21 (m, 2H), 7.29 (d, J=8.4 Hz, 2H), 7.09 (br, 2H), 6.74 (d, J=8.8 Hz, 2H), 6.13 (s, 1H), 4.34-4.33 (m, 2H), 4.00-3.97 (m, 2H), 3.36-3.34 (m, 2H); LC/MS(ESI-MS): [M+H]+=386.2.

### Example 157. 4-(1-(6-amino-9-(pyridin-4-yl)-9H-purin-2-yl)piperidin-4-yl)phenol

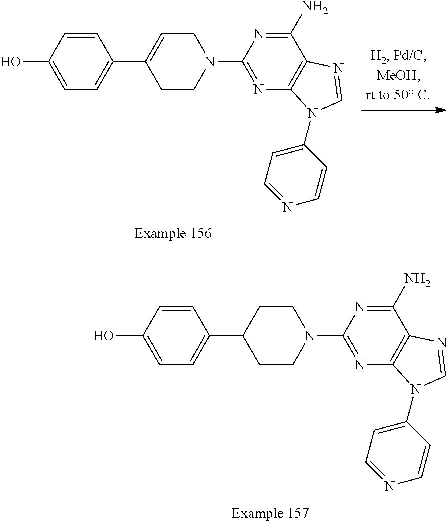 [SMILES: Nc1nc(N2CC=C(c3ccc(O)cc3)CC2)nc2c1ncn2-c1ccncc1 ; Nc1nc(N2CCC(c3ccc(O)cc3)CC2)nc2c1ncn2-c1ccncc1]

To a solution of 4-(1-(6-amino-9-(pyridin-4-yl)-9H-purin-2-yl)-1,2,3,6-tetrahydropyridin-4-yl)phenol (15 mg, 0.04 mmol, 1.0 eq) in MeOH (3 mL), was added palladium on carbon (15 mg, 10%). The reaction was heated under a balloon pressure of hydrogen at 50° C. for 15 hours. Filtered off the solid and evaporate the solvent in the filtrate under reduced pressure. The residue was purified by preparative HPLC (2 mg, 13% yield) as a white solid.

¹H NMR (400 MHz, DMSO-d6): δ 9.15 (s, 1H), 8.77-8.76 (m, 2H), 8.54 (s, 1H), 8.26 (d, J=6.4 Hz, 2H), 7.04 (d, J=8.4 Hz, 2H), 6.67 (d, J=8.8 Hz, 2H), 4.86-4.83 (m, 2H), 2.92-2.86 (m, 2H), 2.70-2.64 (m, 2H), 1.81-1.78 (m, 2H), 1.57-1.47 (m, 2H); LC/MS(ESI-MS): [M+H]+=388.2.

### Example 158. 1-(9-(2-hydroxyethyl)-6-(1H-indazol-1-yl)-9H-purin-2-yl)-4-(4-hydroxyphenyl)piperidin-4-ol

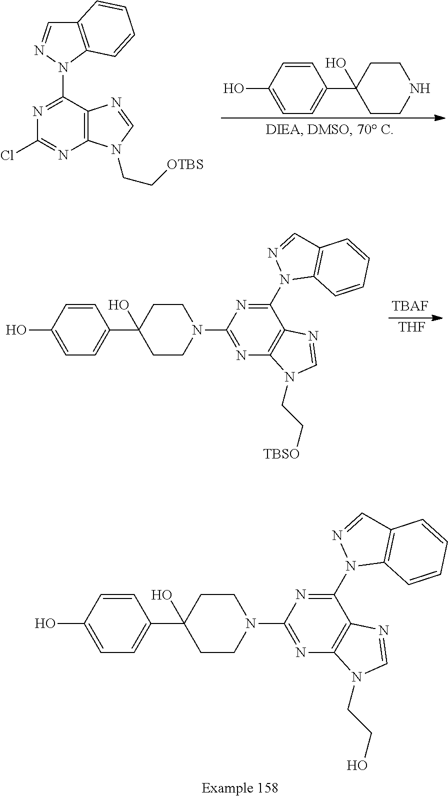

### Step 1

A mixture of 9-(2-((tert-butyldimethylsilyl)oxy)ethyl)-2-chloro-6-(1H-indazol-1-yl)-9H-purine (300 mg, 0.70 mmol, 1.0 eq), 4-(4-hydroxyphenyl)piperidin-4-ol (338 mg, 1.8 mmol, 2.5 eq) and DIEA (272 mg, 2.1 mmol, 3.0 eq) in DMSO (3 mL) was heated at 70° C. for 12 hours. Water (10 mL) was added to afford a precipitate. Filtered the mixture and collected the solid to give 1-(9-(2-((tert-butyldimethylsilyl)oxy)ethyl)-6-(1H-indazol-1-yl)-9H-purin-2-yl)-4-(4-hydroxyphenyl)piperidin-4-ol (280 mg, 68% yield) as a pink solid.

### Step 2

To a solution of 1-(9-(2-((tert-butyldimethylsilyl)oxy)ethyl)-6-(1H-indazol-1-yl)-9H-purin-2-yl)-4-(4-hydroxyphenyl)piperidin-4-ol (280 mg, 0.435 mmol, 1.0 eq) in THF (5 mL) were added TBAF (341 mg, 1.35 mmol, 3.0 eq). The reaction was stirred at room temperature for 12 hours. The reaction mixture was extracted with ethyl acetate (3×20 mL). The combined organic layers were dried with sodium sulfate, filtered and evaporated to dryness in vacuum. The residue was purified by preparative HPLC to get 1-(9-(2-hydroxyethyl)-6-(1H-indazol-1-yl)-9H-purin-2-yl)-4-(4-hydroxyphenyl)piperidin-4-ol (130 mg, 57% yield) as a white solid.

¹H NMR (400 MHz, DMSO-d6): δ9.19 (s, 1H), 8.59-8.55 (m, 2H), 8.10 (s, 1H), 7.94 (d, J=8.0 Hz, 1H), 7.37 (t, J=7.2 Hz, 1H), 7.29 (d, J=8.4 Hz, 2H), 6.68 (d, J=8.8 Hz, 2H), 5.02-4.97 (m, 2H), 4.65-4.62 (m, 2H), 4.20 (t, J=5.6 Hz, 2H), 2.82-3.80 (m, 2H), 3.50-3.44 (m, 2H), 1.99-1.91 (m, 2H), 1.76-1.73 (m, 2H); LC/MS(ESI-MS): [M+H]+=472.4.

### Example 159. 4-(1-(9-(2-hydroxyethyl)-6-(1H-indazol-1-yl)-9H-purin-2-yl)-1,2,3,6-tetrahydropyridin-4-yl)phenol

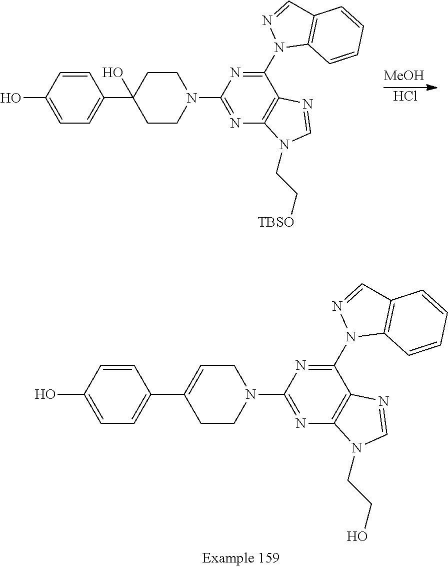 [SMILES: "MeOH" ; CC(C)(C)[Si](C)(C)OCCn1cnc2c(-n3ncc4ccccc43)nc(N3CCC(O)(c4ccc(O)cc4)CC3)nc21 ; Cl ; OCCn1cnc2c(-n3ncc4ccccc43)nc(N3CC=C(c4ccc(O)cc4)CC3)nc21]

To a solution of 1-(9-(2-((tert-butyldimethylsilyl)oxy)ethyl)-6-(1H-indazol-1-yl)-9H-purin-2-yl)-4-(4-hydroxyphenyl)piperidin-4-ol (30 mg, 0.341 mmol, 1.0 eq) in MeOH (3 mL), were added aqueous HCl (1 mL, 2 N), and the reaction was stirred at room temperature for 30 minutes. The reaction mixture was extracted with ethyl acetate (3×20 mL). The combined the organic layers were dried over sodium sulfate, filtered and evaporated to dryness in vacuum. The residue was purified by preparative HPLC to get 4-(1-(9-(2-hydroxyethyl)-6-(1H-indazol-1-yl)-9H-purin-2-yl)-1,2,3,6-tetrahydropyridin-4-yl)phenol (2 mg, 1.3% yield) as a white solid.

¹H NMR (400 MHz, DMSO-d6): δ8.67 (d, J=8.4 Hz, 1H), 8.57 (s, 1H), 8.12 (s, 1H), 7.95 (d, J=7.6 Hz, 1H), 7.65 (t, J=7.6 Hz, 1H), 7.41-7.31 (m, 3H), 6.74 (d, J=8.4 Hz, 2H), 6.21 (s, 1H), 4.47 (s, 2H), 4.22-4.07 (m, 4H), 3.32 (s, 2H), 2.63 (s, 2H); LC/MS(ESI-MS): [M+H]+=454.2.

### Example 160. 5-(2-(4-hydroxy-4-(4-hydroxyphenyl)piperidin-1-yl)-6-methyl-9H-purin-9-yl)pentanoic acid

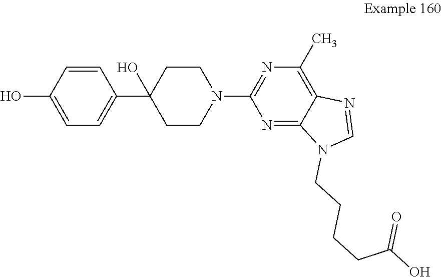 [SMILES: Cc1nc(N2CCC(O)(c3ccc(O)cc3)CC2)nc2c1ncn2CCCCC(=O)O]

The proceeding example was prepared according to Example 21 by substituting ethyl 2-bromoacetate with methyl 5-bromovalerate, substituting 6-(piperazin-1-yl)pyridin-3-ol hydrochloride with 4-(4-hydroxyphenyl)piperidin-4-ol, 2-chloro-6-(1H-indazol-1-yl)-9H-purine with 2-chloro-6-methyl-9H-purine. The desired product 5-(2-(4-hydroxy-4-(4-hydroxyphenyl)piperidin-1-yl)-6-methyl-9H-purin-9-yl)pentanoic acid was isolated as a white solid.

¹H NMR (400 MHz, DMSO-d6): δ8.04 (s, 1H), 7.27-7.24 (m, 2H), 6.68 (d, J=8.4 Hz, 2H), 4.93 (br, JH), 4.61-4.58 (m, 2H), 4.07-4.01 (m, 2H), 3.29-3.19 (m, 2H), 3.01-2.91 (m, 1H), 2.60 (s, 3H), 2.47-2.43 (m, 1H), 2.13-2.08 (m, 2H), 1.86-1.75 (m, 4H), 1.67-1.64 (m, 2H), 1.44-1.38 (m, 2H); LC/MS(ESI-MS): [M+H]+=426.1.

### Example 161. 2-(2-(3-hydroxy-3-phenylpyrrolidin-1-yl)-6-(1H-indazol-1-yl)-9H-purin-9-yl)acetic acid

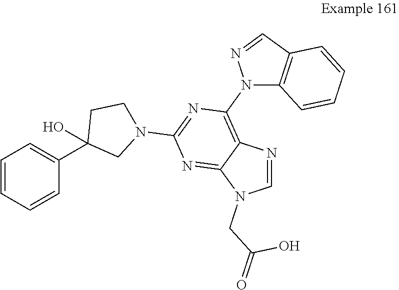 [SMILES: O=C(O)Cn1cnc2c(-n3ncc4ccccc43)nc(N3CCC(O)(c4ccccc4)C3)nc21]

The proceeding example was prepared according to Example 21 by substituting, substituting 6-(piperazin-1-yl)pyridin-3-ol hydrochloride with 3-phenylpyrrolidin-3-ol. The desired product 2-(2-(3-hydroxy-3-phenylpyrrolidin-1-yl)-6-(1H-indazol-1-yl)-9H-purin-9-yl)acetic acid was isolated as a white solid.

¹H NMR (400 MHz, DMSO-d6): δ 13.26 (br, 1H), 8.91-8.63 (m, 2H), 8.10 (s, 1H), 7.95 (s, 1H), 7.74-7.51 (m, 3H), 7.42-7.28 (m, 4H), 5.54 (s, 1H), 4.99-4.80 (m, 2H), 4.13-3.68 (m, 6H); LC/MS(ESI-MS): [M+H]+=456.2.

### Example 162. Methyl (2-(4-(4-hydroxyphenyl)piperazin-1-yl)-9-(pyridin-4-yl)-9H-purin-6-yl)glycinate

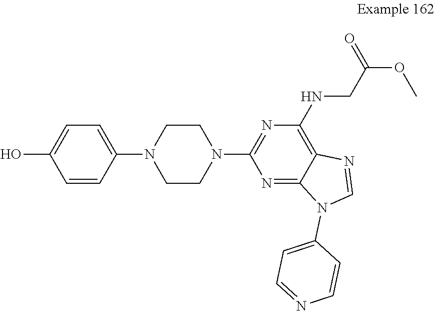 [SMILES: COC(=O)CNc1nc(N2CCN(c3ccc(O)cc3)CC2)nc2c1ncn2-c1ccncc1]

The proceeding example was prepared according to Example 90 by substituting benzylamine with methyl glycinate, pyridin-3-ylboronic acid with pyridin-4-ylboronic acid. The desired product Methyl (2-(4-(4-hydroxyphenyl)piperazin-1-yl)-9-(pyridin-4-yl)-9H-purin-6-yl)glycinate was isolated as a white solid.

¹H NMR (400 MHz, DMSO-d6): δ 8.90-8.81 (m, 2H), 8.73-8.67 (m, 1H), 8.53-8.42 (m, 2H), 8.30-8.20 (m, 1H), 7.19-7.07 (m, 2H), 6.77 (d, J=8.0 Hz, 2H), 4.14 (d, J=4.0 Hz, 2H), 4.11-3.78 (m, 4H), 3.39-3.22 (m, 4H); LC/MS(ESI-MS): [M+H]+=461.2.

### Example 163. (2-(4-(4-hydroxyphenyl)piperazin-1-yl)-9-(pyridin-4-yl)-9H-purin-6-yl)glycine

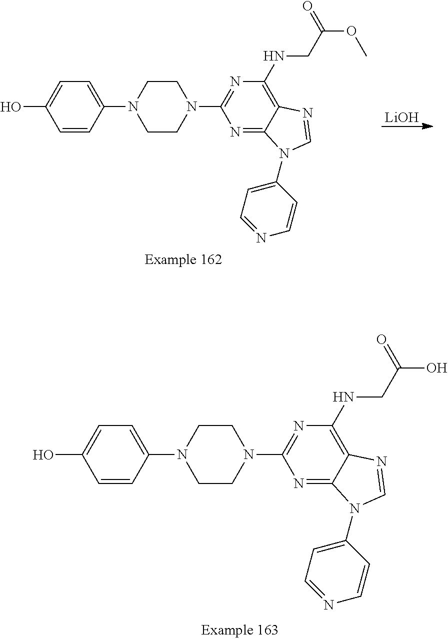 [SMILES: COC(=O)CNc1nc(N2CCN(c3ccc(O)cc3)CC2)nc2c1ncn2-c1ccncc1 ; "LiOH" ; O=C(O)CNc1nc(N2CCN(c3ccc(O)cc3)CC2)nc2c1ncn2-c1ccncc1]

To a solution of methyl (2-(4-(4-hydroxyphenyl)piperazin-1-yl)-9-(pyridin-4-yl)-9H-purin-6-yl)glycinate (60 mg, 0.14 mmol, 1.0 eq) in mixture of water (2 mL) and methanol (3 mL), lithium hydroxide monohydrate (30 mg, 0.71 mmol, 5 eq) was added and the reaction mixture was stirred at room temperature for 2 hours. The pH of the mixture was adjusted to 3 with aqueous 2N HCl to afford a solid. Collected the solid by filtration. The crude compound was purified with preparative HPLC to give (2-(4-(4-hydroxyphenyl)piperazin-1-yl)-9-(pyridin-4-yl)-9H-purin-6-yl)glycine (14 mg, 23% yield) as a white solid. ¹H NMR (400 MHz, DMSO-d6): δ 12.51 (br, 1H), 9.33 (br, 1H), 8.81 (d, J=6.8 Hz, 2H), 8.65 (s, 1H), 8.37-8.35 (m, 2H), 8.06 (s, 1H), 7.20-7.01 (m, 1H), 6.75 (d, J=8.0 Hz, 2H), 4.07-3.83 (m, 7H), 3.33-3.17 (m, 4H); LC/MS(ESI-MS): [M+H]+=447.2.

### Example 164. 1-(2-(4-(4-hydroxyphenyl)piperazin-1-yl)-9-(pyridin-4-yl)-9H-purin-6-yl)pyrrolidine-3-carboxylic acid

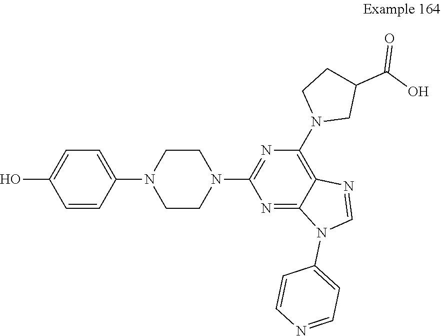 [SMILES: O=C(O)C1CCN(c2nc(N3CCN(c4ccc(O)cc4)CC3)nc3c2ncn3-c2ccncc2)C1]

The proceeding example was prepared according to Example 163 by substituting methyl glycinate with pyrrolidine-3-carboxylate. The desired product 1-(2-(4-(4-hydroxyphenyl)piperazin-1-yl)-9-(pyridin-4-yl)-9H-purin-6-yl)pyrrolidine-3-carboxylic acid was isolated as a brown solid.

¹H NMR (400 MHz, DMSO-d6): δ 8.71 (br, 2H), 8.48 (s, 1H), 8.21-8.08 (m, 2H), 6.84 (d, J=8.8 Hz, 2H), 6.66 (d, J=8.8 Hz, 2H), 4.22-4.08 (m, 2H), 4.03-3.97 (m, 1H), 3.94-3.78 (m, 6H), 3.70-3.58 (m, 1H), 3.11-2.93 (m, 4H), 2.23-2.06 (m, 2H); LC/MS(ESI-MS): [M+H]+=487.2.

### Example 165. 3-((2-(4-(4-hydroxyphenyl)piperazin-1-yl)-9-(pyridin-4-yl)-9H-purin-6-yl)(methyl)amino)propanoic acid

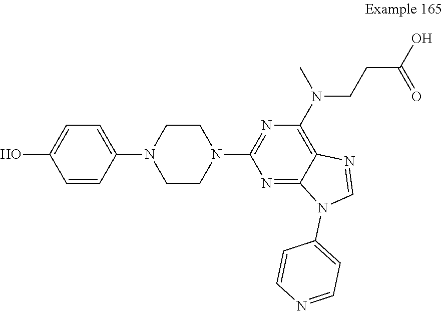 [SMILES: CN(CCC(=O)O)c1nc(N2CCN(c3ccc(O)cc3)CC2)nc2c1ncn2-c1ccncc1]

The proceeding example was prepared according to Example 163 by substituting methyl glycinate with methyl 3-(methyl amino)propanoate. The desired product 3-((2-(4-(4-hydroxyphenyl)piperazin-1-yl)-9-(pyridin-4-yl)-9H-purin-6-yl)(methyl)amino) propanoic acid was isolated as a yellow solid.

¹H NMR (400 MHz, DMSO-d6): δ 12.27 (s, 1H), 8.86 (s, 1H), 8.73 (d, J=6.4 Hz, 2H), 8.52 (s, 1H), 8.16 (d, J=6.4 Hz, 2H), 6.86-6.79 (m, 2H), 6.67 (d, J=8.8 Hz, 2H), 4.36-4.10 (m, 1H), 3.89-3.81 (m, 4H), 3.79-3.53 (m, 1H), 3.21 (s, 3H), 3.19-3.08 (m, 1H), 3.07-3.00 (m, 4H), 2.67-2.62 (m, 2H); LC/MS(ESI-MS): [M+H]+=475.2.

### Example 166. 3N-(2-(4-(4-hydroxyphenyl)piperazin-1-yl)-9-(pyridin-4-yl)-9H-purin-6-yl)-N-methylglycine

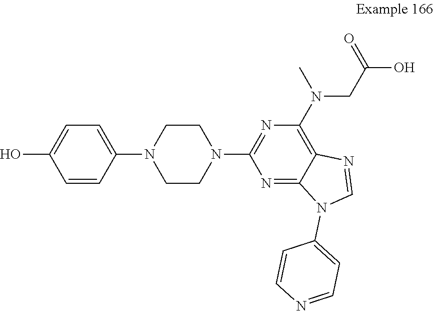 [SMILES: CN(CC(=O)O)c1nc(N2CCN(c3ccc(O)cc3)CC2)nc2c1ncn2-c1ccncc1]

The proceeding example was prepared according to Example 163 by substituting methyl glycinate with methyl methylglycinate. The desired product 3N-(2-(4-(4-hydroxyphenyl)piperazin-1-yl)-9-(pyridin-4-yl)-9H-purin-6-yl)-N-methylglycine was isolated as a yellow solid.

¹H NMR (400 MHz, DMSO-d6): δ 8.89-8.88 (m, 2H), 8.70-8.60 (m, 1H), 8.50-8.40 (m, 2H); 7.30-7.20 (m, 2H), 6.81-6.70 (m, 2H), 5.10-4.98 (m, 1H), 4.32-4.20 (m, 1H), 4.20-3.90 (m, 4H), 3.70-3.60 (m, 1.5H), 3.60-3.30 (m, 4H), 3.20-3.10 (m, 1.5H); LC/MS(ESI-MS). [M+H]+=461.2.

### Example 167. (2-(4-(4-hydroxyphenyl)piperazin-1-yl)-9-(pyridin-4-yl)-9H-purin-6-yl)proline

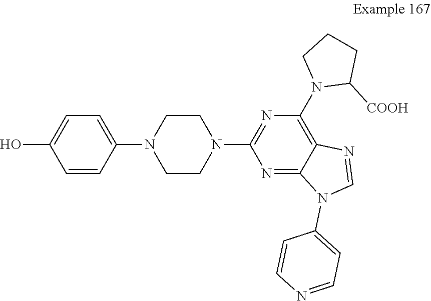 [SMILES: O=C(O)C1CCCN1c1nc(N2CCN(c3ccc(O)cc3)CC2)nc2c1ncn2-c1ccncc1]

The proceeding example was prepared according to Example 163 by substituting methyl glycinate with methyl prolinate. The desired product (2-(4-(4-hydroxyphenyl) piperazin-1-yl)-9-(pyridin-4-yl)-9H-purin-6-yl)proline was isolated as a yellow solid.

¹H NMR (400 MHz, DMSO-d6): δ12.48 (br, 1H), 8.85 (s, 1H), 8.74-7.72 (m, 2H), 8.54-8.49 (m, 1H), 8.17 (d, J=5.6 Hz, 2H), 6.86-6.84 (m, 2H), 6.66 (d, J=9.2 Hz, 2H), 5.31-5.29 (m, 0.3H), 4.56-4.53 (m, 0.7H), 4.21-4.09 (m, 1.5H), 3.88-3.77 (m, 4H), 3.77-3.68 (m, 0.5H), 3.55-2.91 (m, 4H), 2.50-1.79 (m, 4H); LC/MS(ESI-MS): [M+H]+=487.2.

### Example 168. 3-((2-hydroxyethyl)(2-(4-(4-hydroxyphenyl)piperazin-1-yl)-9-(pyridin-4-yl)-9H-purin-6-yl)amino)propanoic acid

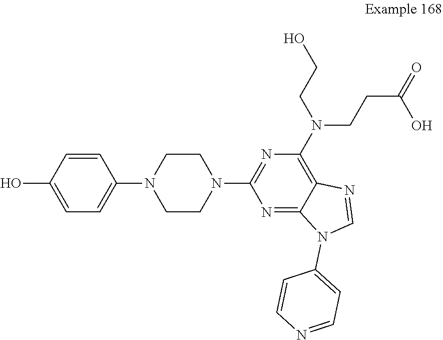 [SMILES: O=C(O)CCN(CCO)c1nc(N2CCN(c3ccc(O)cc3)CC2)nc2c1ncn2-c1ccncc1]

The proceeding example was prepared according to Example 163 by substituting methyl glycinate with methyl 3-((2-hydroxyethyl)amino)propanoate. The desired product 3-((2-hydroxyethyl)(2-(4-(4-hydroxyphenyl)piperazin-1-yl)-9-(pyridin-4-yl)-9H-purin-6-yl)amino)propanoic acid was isolated as a white solid.

¹H NMR (400 MHz, DMSO-d6): δ 8.85 (d, J=6.0 Hz, 2H), 8.62 (br, 1H), 8.41 (d, J=6.0 Hz, 2H), 7.24 (br, 2H), 6.77 (d, J=8.4 Hz, 2H), 4.31-4.17 (m, 6H), 3.70-3.67 (m, 6H), 3.29-3.11 (m, 4H), 2.71-2.67 (m, 2H); LC/MS(ESI-MS): [M+H]+=505.3.

### Example 169. 4-(4-(6-((1-hydroxybutan-2-yl)(methyl)amino)-9-(pyridin-3-yl)-9H-purin-2-yl)piperazin-1-yl)phenol

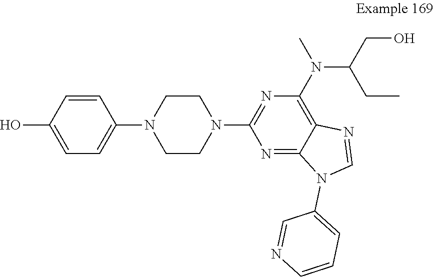 [SMILES: CCC(CO)N(C)c1nc(N2CCN(c3ccc(O)cc3)CC2)nc2c1ncn2-c1cccnc1]

The proceeding example was prepared according to Example 163 by substituting methyl glycinate with 2-(methylamino)butan-1-ol, pyridin-4-ylboronic acid with pyridin-3-ylboronic acid. The desired product 4-(4-(6-((1-hydroxybutan-2-yl)(methyl)amino)-9-(pyridin-3-yl)-9H-purin-2-yl)piperazin-1-yl)phenol was isolated as a white solid.

¹H NMR (400 MHz, DMSO-d6): δ 8.87 (s, 1H), 8.73 (d, J=6.0 Hz, 2H), 8.48 (s, 1H), 8.16 (d, J=6.4 Hz, 2H), 6.85 (d, J=8.8 Hz, 2H), 6.68 (d, J=8.8 Hz, 2H), 5.87 (br, 1H), 4.71-4.69 (m, 1H), 3.95-3.81 (m, 4H), 3.64-3.51 (m, 3H), 3.09-2.91 (m, 4H), 1.66-1.55 (m, 2H), 0.84 (t, J=7.6 Hz, 3H); LC/MS(ESI-MS): [M+H]+=475.32.

### Example 170. 1-(2-(4-(4-hydroxyphenyl)piperazin-1-yl)-9-(pyridin-4-yl)-9H-purin-6-yl)piperidine-3-carboxylic acid

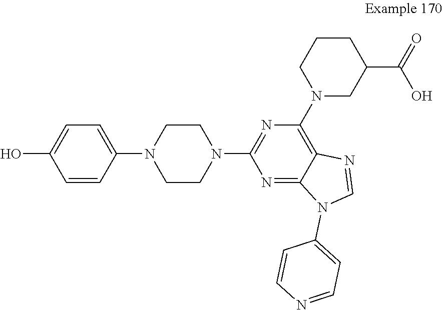 [SMILES: O=C(O)C1CCCN(c2nc(N3CCN(c4ccc(O)cc4)CC3)nc3c2ncn3-c2ccncc2)C1]

The proceeding example was prepared according to Example 163 by substituting methyl glycinate with methyl piperidine-3-carboxylate. The desired product 1-(2-(4-(4-hydroxyphenyl)piperazin-1-yl)-9-(pyridin-4-yl)-9H-purin-6-yl)piperidine-3-carboxylic acid was isolated as a brown solid.

¹H NMR (400 MHz, DMSO-d6): δ 9.14 (br, 1H), 8.90-8.77 (m, 2H), 8.59 (s, 1H), 8.31-8.22 (m, 2H), 7.11-6.99 (m, 2H), 6.73 (d, J=8.4 Hz, 2H), 3.99-3.81 (m, 4H), 3.39-3.26 (m, 2H), 3.21-3.12 (m, 4H), 3.11-3.07 (m, 1H), 2.05-1.97 (m, 1H), 1.83-1.53 (m, 2H), 1.54-1.51 (m, 1H), 1.24-1.17 (m, 1H); LC/MS(ESI-MS): [M+H]+=501.2.

### Example 171. (3S,4S)-1-(2-(4-(4-hydroxyphenyl)piperazin-1-yl)-9-(pyridin-4-yl)-9H-purin-6-yl)pyrrolidine-3,4-diol

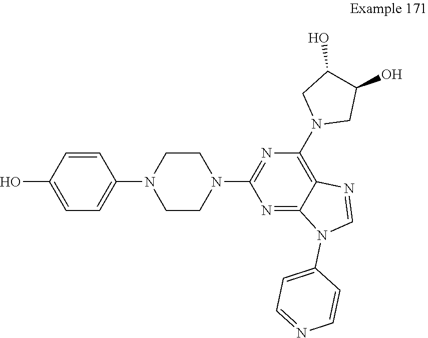 [SMILES: Oc1ccc(N2CCN(c3nc(N4C[C@H](O)[C@@H](O)C4)c4ncn(-c5ccncc5)c4n3)CC2)cc1]

The proceeding example was prepared according to Example 163 by substituting methyl glycinate with (3S,4S)-pyrrolidine-3,4-diol. The desired product (3S,4S)-1-(2-(4-(4-hydroxyphenyl)piperazin-1-yl)-9-(pyridin-4-yl)-9H-purin-6-yl)pyrrolidine-3,4-diol was isolated as a brown solid.

¹H NMR (400 MHz, DMSO-d6): δ 8.85 (s, 1H), 8.72 (d, J=5.6 Hz, 2H), 8.50 (s, 1H), 8.18 (d, J=5.6 Hz, 2H), 6.85 (d, J=8.8 Hz, 2H), 6.68 (d, J=8.8 Hz, 2H), 5.19-5.16 (m, 2H), 4.19-4.09 (m, 2H), 4.05-3.95 (m, 2H), 3.88-3.81 (m, 4H), 3.79-3.68 (m, 2H), 3.11-2.96 (m, 4H); LC/MS(ESI-MS): [M+H]+=475.2.

### Example 172. 1-(2-(4-(4-hydroxyphenyl)piperazin-1-yl)-9-(pyridin-4-yl)-9H-purin-6-yl)-2-methylpyrrolidine-2-carboxylic acid

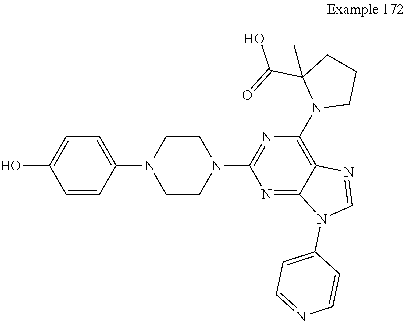 [SMILES: CC1(C(=O)O)CCCN1c1nc(N2CCN(c3ccc(O)cc3)CC2)nc2c1ncn2-c1ccncc1]

The proceeding example was prepared according to Example 163 by substituting methyl glycinate with methyl 2-methylpyrrolidine-2-carboxylate. The desired product 1-(2-(4-(4-hydroxyphenyl)piperazin-1-yl)-9-(pyridin-4-yl)-9H-purin-6-yl)-2-methylpyrrolidine-2-carboxylic acid was isolated as a gray solid.

¹H NMR (400 MHz, DMSO-d6): δ 12.23 (br, 1H), 9.45 (br, 1H), 8.86 (d, J=5.6 Hz, 2H), 8.63 (s, 1H), 8.43 (d, J=6.0 Hz, 2H), 7.14 (br, 2H), 6.77 (d, J=8.8 Hz, 2H), 4.39-4.36 (m, 1H), 4.11-4.05 (m, 1H), 4.03-3.78 (m, 4H), 3.35-3.13 (m, 4H), 2.23-1.93 (m, 4H), 1.61 (s, 3H); LC/MS(ESI-MS): [M+H]+=501.3.

### Example 173. 2-((2-(4-(4-hydroxyphenyl)piperazin-1-yl)-9-(pyridin-4-yl)-9H-purin-6-yl)amino)propane-1,3-diol

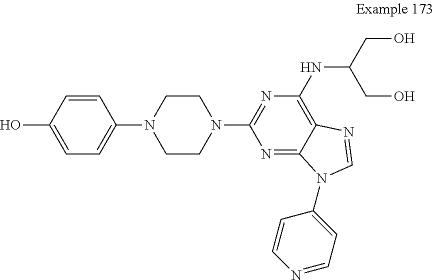 [SMILES: OCC(CO)Nc1nc(N2CCN(c3ccc(O)cc3)CC2)nc2c1ncn2-c1ccncc1]

The proceeding example was prepared according to Example 163 by substituting methyl glycinate with 2-aminopropane-1,3-diol. The desired product 2-((2-(4-(4-hydroxyphenyl)piperazin-1-yl)-9-(pyridin-4-yl)-9H-purin-6-yl)amino)propane-1,3-diol was isolated as a white solid.

¹H NMR (400 MHz, DMSO-d6): δ 8.99-8.85 (m, 2H), 8.71 (s, 1H), 8.60-8.49 (m, 4H), 7.31-7.11 (m, 3H), 6.80 (d, J=8.0 Hz, 2H), 4.26-4.24 (m, 2H), 4.19-3.87 (m, 4H), 3.65-3.57 (m, 4H), 3.47-3.28 (m, 4H); LC/MS(ESI-MS): [M+H]+=463.2.

### Example 174. 4-(2-(4-(4-hydroxyphenyl)piperazin-1-yl)-9-(pyridin-4-yl)-9H-purin-6-yl)piperazin-2-one

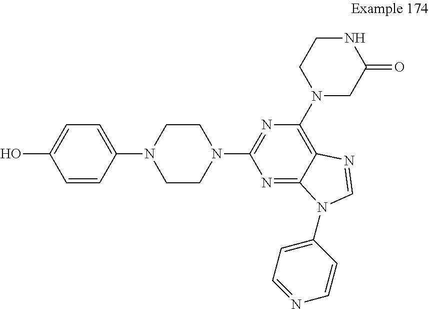 [SMILES: O=C1CN(c2nc(N3CCN(c4ccc(O)cc4)CC3)nc3c2ncn3-c2ccncc2)CCN1]

The proceeding example was prepared according to Example 163 by substituting methyl glycinate with piperazin-2-one. The desired product 4-(2-(4-(4-hydroxyphenyl) piperazin-1-yl)-9-(pyridin-4-yl)-9H-purin-6-yl)piperazin-2-one was isolated as a white solid.

¹H NMR (400 MHz, DMSO-d6): δ8.87 (s, 1H), 8.73 (d, J=5.6 Hz, 2H), 8.56 (s, 1H), 8.15 (d, J=6.0 Hz, 2H), 6.85 (d, J=8.8 Hz, 2H), 6.68 (d, J=8.8 Hz, 2H), 4.80-4.21 (m, 4H), 3.91-3.70 (m, 6H), 3.12-2.98 (m, 4H); LC/MS(ESI-MS): [M+H]+=472.2.

### Example 175. 4-(2-(4-(4-hydroxyphenyl)piperazin-1-yl)-9-(pyridin-4-yl)-9H-purin-6-yl)-1-methylpiperazin-2-one

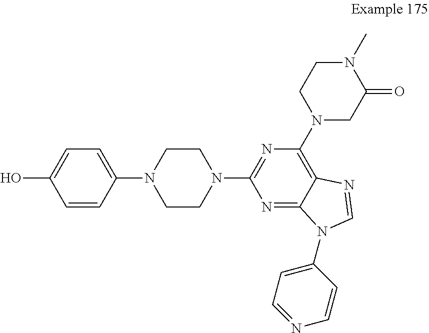 [SMILES: CN1CCN(c2nc(N3CCN(c4ccc(O)cc4)CC3)nc3c2ncn3-c2ccncc2)CC1=O]

The proceeding example was prepared according to Example 163 by substituting methyl glycinate with 1-methylpiperazin-2-one. The desired product 4-(2-(4-(4-hydroxyphenyl)piperazin-1-yl)-9-(pyridin-4-yl)-9H-purin-6-yl)-1-methylpiperazin-2-one was isolated as a white solid.

¹H NMR (400 MHz, DMSO-d6): δ8.86 (s, 1H), 8.73 (d, J=5.6 Hz, 2H), 8.57 (s, 1H), 8.14 (d, J=5.6 Hz, 2H), 6.85 (d, J=8.8 Hz, 2H), 6.67 (d, J=8.8 Hz, 2H), 4.80-4.21 (m, 4H), 3.91-3.78 (m, 4H), 3.47 (t, J=5.6 Hz, 2H), 3.12-2.98 (m, 4H), 2.90 (s, 3H); LC/MS(ESI-MS): [M+H]+=486.2.

### Example 176. (S)-4-(4-(6-((1-(benzyloxy)-3-hydroxypropan-2-yl)amino)-9-(pyridin-4-yl)-9H-purin-2-yl)piperazin-1-yl)phenol

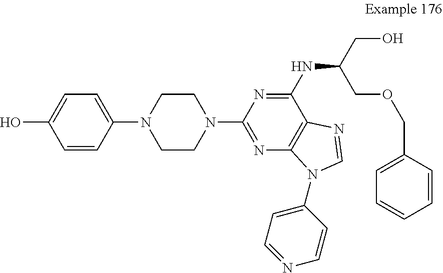 [SMILES: OC[C@@H](COCc1ccccc1)Nc1nc(N2CCN(c3ccc(O)cc3)CC2)nc2c1ncn2-c1ccncc1]

The proceeding example was prepared according to Example 163 by substituting methyl glycinate with (S)-2-amino-3-(benzyloxy)propan-1-ol. The desired product (S)-4-(4-(6-((1-(benzyloxy)-3-hydroxypropan-2-yl)amino)-9-(pyridin-4-yl)-9H-purin-2-yl)piperazin-1-yl)phenol was isolated as a white solid.

¹H NMR (400 MHz, DMSO-d6): δ8.87 (d, J=5.6 Hz, 2H), 8.69 (s, 1H), 8.48 (d, J=4.8 Hz, 2H), 7.33-7.13 (m, 8H), 6.82-6.77 (m, 2H), 4.57-4.50 (m, 3H), 4.13-3.85 (m, 3H), 3.67-3.58 (m, 4H), 3.31-3.14 (m, 5H); LC/MS(ESI-MS): [M+H]+=553.2.

### Example 177. 2-((2-(4-(4-hydroxyphenyl)piperazin-1-yl)-9-(pyridin-4-yl)-9H-purin-6-yl)amino)-2-methylpropane-1,3-diol

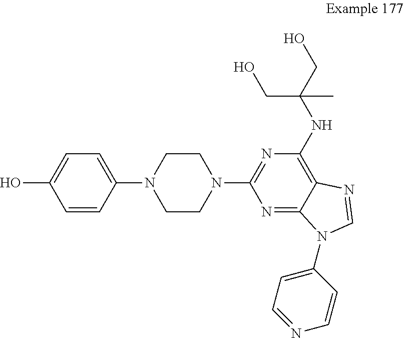 [SMILES: CC(CO)(CO)Nc1nc(N2CCN(c3ccc(O)cc3)CC2)nc2c1ncn2-c1ccncc1]

The proceeding example was prepared according to Example 163 by substituting methyl glycinate with 2-amino-2-methylpropane-1,3-diol. The desired product 2-((2-(4-(4-hydroxyphenyl)piperazin-1-yl)-9-(pyridin-4-yl)-9H-purin-6-yl)amino)-2-methylpropane-1,3-diol was isolated as a yellow solid.

¹H NMR (400 MHz, DMSO-d6): δ9.12 (s, 1H), 8.82 (d, J=6.4 Hz, 2H), 8.63 (s, 1H), 8.37 (d, J=6.4 Hz, 2H), 7.16-7.03 (m, 2H), 6.75 (d, J=8.8 Hz, 2H), 6.39 (s, 1H), 4.03-3.93 (m, 4H), 3.77 (d, J=10.8 Hz, 2H), 3.64 (d, J=10.8 Hz, 2H), 3.24-3.17 (m, 4H), 1.43 (s, 3H); LC/MS(ESI-MS): [M+H]+=477.2.

### Example 178. 4-(4-(6-(piperazin-1-yl)-9-(pyridin-4-yl)-9H-purin-2-yl)piperazin-1-yl)phenol

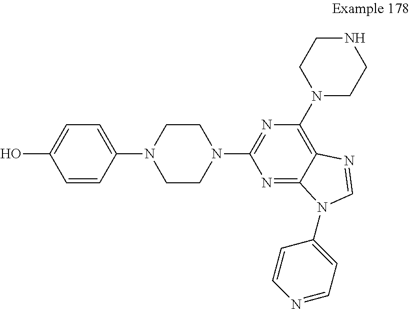 [SMILES: Oc1ccc(N2CCN(c3nc(N4CCNCC4)c4ncn(-c5ccncc5)c4n3)CC2)cc1]

The proceeding example was prepared according to Example 163 by substituting methyl glycinate with piperazine. The desired product 2-((2-(4-(4-hydroxyphenyl) piperazin-1-yl)-9-(pyridin-4-yl)-9H-purin-6-yl)amino)-2-methylpropane-1,3-diol was isolated as a white solid.

¹H NMR (400 MHz, DMSO-d6): δ 8.86 (br, 1H), 8.74-8.72 (m, 2H), 8.51 (s, 1H), 8.16-8.14 (m, 2H), 6.85 (d, J=9.2 Hz, 2H), 6.69-6.66 (m, 2H), 4.21-4.01 (m, 4H), 3.90-3.81 (m, 4H), 3.03 (t, J=4.8 Hz, 4H), 2.87-2.79 (m, 4H); LC/MS(ESI-MS): [M+H]+=458.2.

### Example 179. 4-(4-(6-(4-methylpiperazin-1-yl)-9-(pyridin-4-yl)-9H-purin-2-yl)piperazin-1-yl)phenol

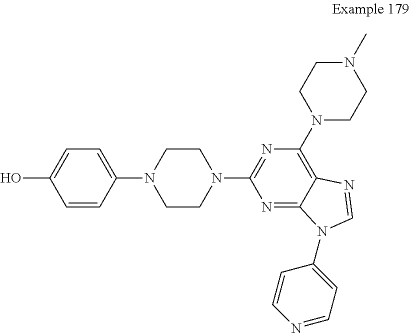 [SMILES: CN1CCN(c2nc(N3CCN(c4ccc(O)cc4)CC3)nc3c2ncn3-c2ccncc2)CC1]

The proceeding example was prepared according to Example 163 by substituting methyl glycinate with 1-methylpiperazine. The desired product 4-(4-(6-(4-methylpiperazin-1-yl)-9-(pyridin-4-yl)-9H-purin-2-yl)piperazin-1-yl)phenol was isolated as a white solid.

¹H NMR (400 MHz, DMSO-d6): δ 9.18 (s, 1H), 8.73-8.71 (m, 2H), 8.49 (s, 1H), 8.16-8.14 (m, 2H), 7.27 (d, J=8.4 Hz, 2H), 6.68 (d, J=8.8 Hz, 2H), 4.88 (s, 1H), 4.52-4.48 (m, 2H), 4.31-3.99 (m, 4H), 3.31-3.25 (m, 2H), 2.50-2.39 (m, 4H), 1.87 (s, 3H), 1.87-1.80 (m, 2H), 1.68-1.65 (m, 2H); LC/MS(ESI-MS): [M+H]+=487.3.

### Example 180. N-methyl-N-(2-(4-phenylpiperazin-1-yl)-9-(pyridin-4-yl)-9H-purin-6-yl)glycine

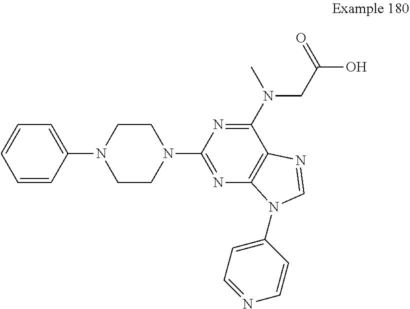 [SMILES: CN(CC(=O)O)c1nc(N2CCN(c3ccccc3)CC2)nc2c1ncn2-c1ccncc1]

The proceeding example was prepared according to Example 163 by substituting methyl glycinate with methyl methylglycinate, 4-(piperazin-1-yl)phenol with 1-phenylpiperazine. The desired product N-methyl-N-(2-(4-phenylpiperazin-1-yl)-9-(pyridin-4-yl)-9H-purin-6-yl)glycine was isolated as a yellow solid.

¹H NMR (400 MHz, DMSO-d6): δ 8.85-8.83 (m, 2H), 8.53 (s, 1H), 8.38 (d, J=6.4 Hz, 2H), 7.27-7.23 (m, 2H), 7.01 (d, J=8.0 Hz, 2H), 6.84-6.81 (m, 1H), 4.90-4.27 (m, 2H), 3.93-3.90 (m, 4H), 5.06 (s, 1H), 4.38 (m, 1H), 3.99-3.83 (m, 4H), 3.75 (s, 1H), 3.68 (s, 3H), 3.29-3.11 (m, 4H); LC/MS(ESI-MS): [M+H]+=445.2.

### Example 181. 1-(2-(4-(pyridin-3-yl)piperazin-1-yl)-9-(pyridin-4-yl)-9H-purin-6-yl)pyrrolidine-3-carboxylic acid

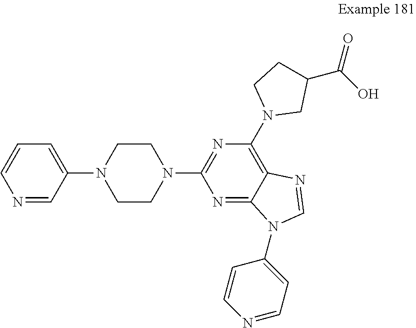 [SMILES: O=C(O)C1CCN(c2nc(N3CCN(c4cccnc4)CC3)nc3c2ncn3-c2ccncc2)C1]

The proceeding example was prepared according to Example 163 by substituting methyl glycinate with methyl pyrrolidine-3-carboxylate, 4-(piperazin-1-yl)phenol with 1-(pyridin-3-yl)piperazine. The desired product N-methyl-N-(2-(4-phenylpiperazin-1-yl)-9-(pyridin-4-yl)-9H-purin-6-yl)glycine was isolated as a yellow solid.

¹H NMR (400 MHz, DMSO-d6): δ 8.73 (br, 1H), 8.49 (s, 1H), 8.23-8.02 (m, 3H), 7.55 (t, J=6.9 Hz, 1H), 6.86 (d, J=8.4 Hz, 1H), 6.67-6.63 (m, 1H), 4.22-3.91 (m, 3H), 3.89-3.78 (m, 4H), 3.69-3.48 (m, 4H), 3.41-3.07 (m, 2H), 2.21-2.01 (m, 2H); LC/MS(ESI-MS). [M+H]+=472.2.

### Example 182. 1-(2-(4-phenylpiperazin-1-yl)-9-(pyridin-4-yl)-9H-purin-6-yl)pyrrolidine-3-carboxylic acid

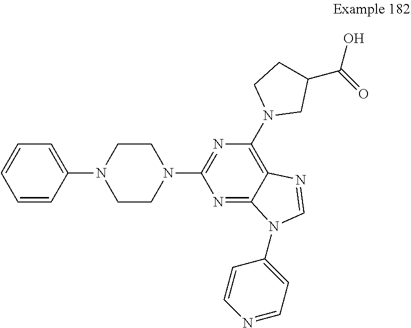 [SMILES: O=C(O)C1CCN(c2nc(N3CCN(c4ccccc4)CC3)nc3c2ncn3-c2ccncc2)C1]

The proceeding example was prepared according to Example 163 by substituting methyl glycinate with methyl pyrrolidine-3-carboxylate, 4-(piperazin-1-yl)phenol with 1-phenylpiperazine. The desired product 1-(2-(4-phenylpiperazin-1-yl)-9-(pyridin-4-yl)-9H-purin-6-yl)pyrolidine-3-carboxylic acid was isolated as a yellow solid.

¹H NMR (400 MHz, DMSO-d6): δ9.13-8.70 (m, 3H), 8.45 (s, 1H), 7.36-7.32 (m, 3H), 7.17 (d, J=8.0 Hz, 2H), 7.04-6.99 (m, 1H), 4.31-3.79 (m, 9H), 3.37-3.35 (m, 4H), 2.39-2.20 (m, 2H); LC/MS(ESI-MS): [M+H]+=471.2.

### Example 183. 4-(4-(6-((1-hydroxybutan-2-yl)(methyl)amino)-9-(pyridin-3-yl)-9H-purin-2-yl)piperazin-1-yl)phenol

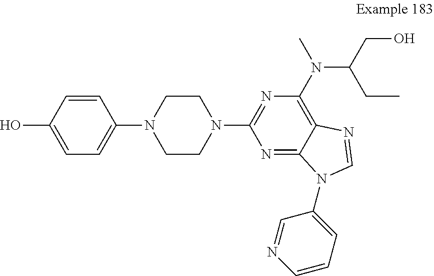 [SMILES: CCC(CO)N(C)c1nc(N2CCN(c3ccc(O)cc3)CC2)nc2c1ncn2-c1cccnc1]

The proceeding example was prepared according to Example 163 by substituting methyl glycinate with 2-(methylamino)butan-1-ol, pyridin-4-ylboronic acid with pyridin-3-ylboronic acid. The desired product 4-(4-(6-((1-hydroxybutan-2-yl)(methyl)amino)-9-(pyridin-3-yl)-9H-purin-2-yl)piperazin-1-yl)phenol was isolated as a yellow solid.

¹H NMR (400 MHz, DMSO-d6): δ 8.87 (s, 1H), 8.73 (d, J=6.0 Hz, 2H), 8.48 (s, 1H), 8.16 (d, J=6.4 Hz, 2H), 6.85 (d, J=8.8 Hz, 2H), 6.68 (d, J=8.8 Hz, 2H), 5.87 (br, 1H), 4.71-4.69 (m, 1H), 3.95-3.81 (m, 4H), 3.64-3.51 (m, 3H), 3.09-2.91 (m, 4H), 1.66-1.55 (m, 2H), 0.84 (t, J=7.6 Hz, 3H); LC/MS(ESI-MS): [M+H]+=475.32

### Example 184 4-(4-(9-(pyridin-4-yl)-9H-purin-2-yl)piperazin-1-yl)phenol

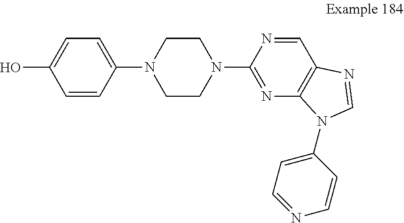 [SMILES: Oc1ccc(N2CCN(c3ncc4ncn(-c5ccncc5)c4n3)CC2)cc1]

The proceeding example was prepared according to Example 43 by substituting 2-chloro-6-methyl-9H-purine with 2-chloro-9H-purine, phenyl boronic acid with 4 pyridin-4-ylboronic acid. The desired 4-(4-(9-(pyridin-4-yl)-9H-purin-2-yl)piperazin-1-yl)phenol was isolated as a yellow solid.

¹H NMR (400 MHz, DMSO-d6): δ 8.88-8.86 (m, 3H), 8.77 (d, J=6.0 Hz, 2H), 8.17 (d, J=6.0 Hz, 2H), 6.86 (d, J=8.8 Hz, 2H), 6.67 (d, J=8.8 Hz, 2H), 3.99-3.89 (m, 4H), 3.10-3.03 (m, 4H); LC/MS(ESI-MS): [M+H]+=374.2.

### Example 185. 4-(2-(4-hydroxy-4-(4-hydroxyphenyl)piperidin-1-yl)-6-methyl-9H-purin-9-yl)benzonitrile

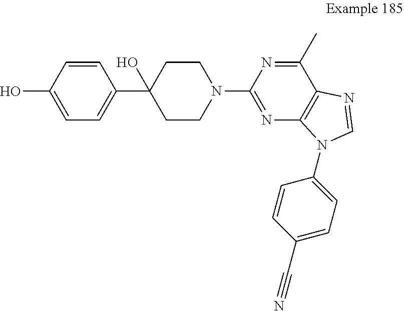 [SMILES: Cc1nc(N2CCC(O)(c3ccc(O)cc3)CC2)nc2c1ncn2-c1ccc(C#N)cc1]

The proceeding example was prepared according to Example 43 by substituting 4-(piperazin-1-yl)phenol with 4-(4-hydroxyphenyl)piperidin-4-ol, phenylboronic acid with 4-cyanophenylboronic acid. The desired 4-(2-(4-hydroxy-4-(4-hydroxyphenyl)piperidin-1-yl)-6-methyl-9H-purin-9-yl)benzonitrile was isolated as a yellow solid.

¹H NMR (400 MHz, DMSO-d6): δ 9.20 (s, 1H), 8.66 (s, 1H), 8.27 (d, J=8.8 Hz, 2H), 8.08 (d, J=8.8 Hz, 2H), 7.26 (d, J=8.8 Hz, 2H), 6.68 (d, J=8.4 Hz, 2H), 4.93 (s, 1H), 4.62-4.59 (m, 2H), 2.59 (s, 3H), 2.51-2.49 (m, 2H), 1.89-1.81 (m, 2H), 1.70-1.67 (m, 2H); LC/MS(ESI-MS): [M+H]+=426.2.

### Example 186. 4-(4-(6-amino-9-(pyridin-4-yl)-9H-purin-2-yl)-2-methylpiperazin-1-yl)phenol

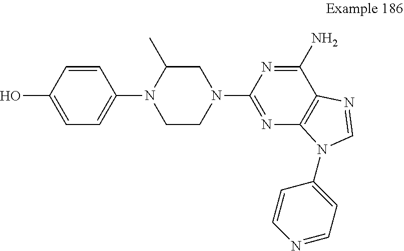 [SMILES: CC1CN(c2nc(N)c3ncn(-c4ccncc4)c3n2)CCN1c1ccc(O)cc1]

The proceeding example was prepared according to Example 66 by substituting 4-(piperazin-1-yl)phenol with 4-(2-methylpiperazin-1-yl)phenol, phenylboronic acid with pyridin-4-ylboronic acid. The desired 4-(4-(6-amino-9-(pyridin-4-yl)-9H-purin-2-yl)-2-methylpiperazin-1-yl)phenol was isolated as a yellow solid.

¹H NMR (400 MHz, DMSO-d6): δ8.93 (s, 1H), 8.71 (d, J=5.2 Hz, 2H), 8.52 (s, 1H), 8.17 (d, J=6.4 Hz, 2H), 7.09 (br, 2H), 6.87 (d, J=8.8 Hz, 2H), 6.68 (d, J=8.4 Hz, 2H), 4.00-3.97 (m, 1H), 3.87-3.85 (m, 1H), 3.76-3.60 (m, 2H), 3.51-3.47 (m, 1H), 2.98-2.96 (m, 2H), 0.84 (d, J=6.4 Hz, 3H); LC/MS(ESI-MS): [M+H]+=403.2.

### Example 187. 4-(4-(6-amino-9-(tetrahydro-2H-pyran-2-yl)-9H-purin-2-yl)piperazin-1-yl)phenol

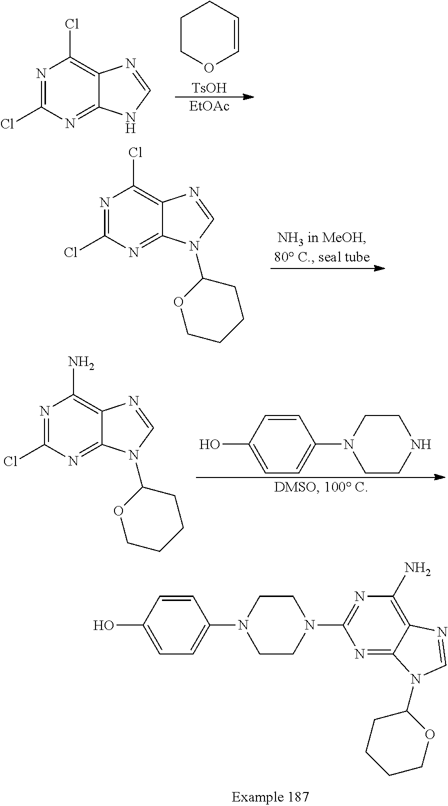

### Step 1

To a solution of 2,6-dichloro-9H-purine (10 g, 52.91 mmol, 1.0 eq) and 3,4-dihydro-2H-pyran (7.5 g, 89.21 mmol, 1.7 eq) in ethyl acetate (20 mL) was added 4-methylbenzenesulfonic acid (0.08 g, 0.4 mmol, 0.008 eq). The reaction mixture was stirred at 50° C. for 2 hours and then at room temperature for 15 hours. The reaction mixture was concentrated. The residue was purified by slurrying (PE:EtOAc=5:1) to get 2,6-dichloro-9-(tetrahydro-2H-pyran-2-yl)-9H-purine (12.5 g, 86% yield) as a white solid.

### Step 2

A mixture of 2,6-dichloro-9-(tetrahydro-2H-pyran-2-yl)-9H-purine (300 mg, 1.10 mmol, 1.0 eq) and ammonia in methanol (5 mL, 7 mol/L, 32.0 eq) in a sealed tube was stirred at 80° C. for 15 hours. The reaction mixture was evaporated to dryness in vacuum. The residue was concentrated to give 2-chloro-9-(tetrahydro-2H-pyran-2-yl)-9H-purin-6-amine (270 mg, 96% yield) as a white solid.

### Step 3

A mixture of 2-chloro-9-(tetrahydro-2H-pyran-2-yl)-9H-purin-6-amine (270 mg, 1.06 mmol, 1.0 eq) and 4-(piperazin-1-yl) phenol (474 mg, 2.66 mmol, 2.5 eq) in DMSO (5 mL) was stirred at 100° C. for 15 hours under N₂. After cooling, the reaction mixture was quenched with water (10 mL). The organic layer was separated and concentrated. The residue was purified by preparative HPLC to give 4-(4-(6-amino-9-(tetrahydro-2H-pyran-2-yl)-9H-purin-2-yl)piperazin-1-yl)phenol (2 mg, 0.5% yield) of desired product as yellow solid. 1HNMR (400 MHz, CD3OD): δ 7.93 (s, 1H), 6.94-6.92 (m, 2H), 6.74-6.72 (m, 2H), 5.61-5.58 (m, 1H), 4.12-4.09 (m, 1H), 3.93-3.91 (m, 4H), 3.79-3.73 (m, 1H), 3.06-3.04 (m, 4H), 2.15-1.98 (m, 3H), 1.78-1.64 (m, 3H); LC/MS(ESI-MS): [M+H]+=396.2.

### Example 188. 4-(4-(6-amino-9-(piperidin-4-yl)-9H-purin-2-yl)piperazin-1-yl)phenol

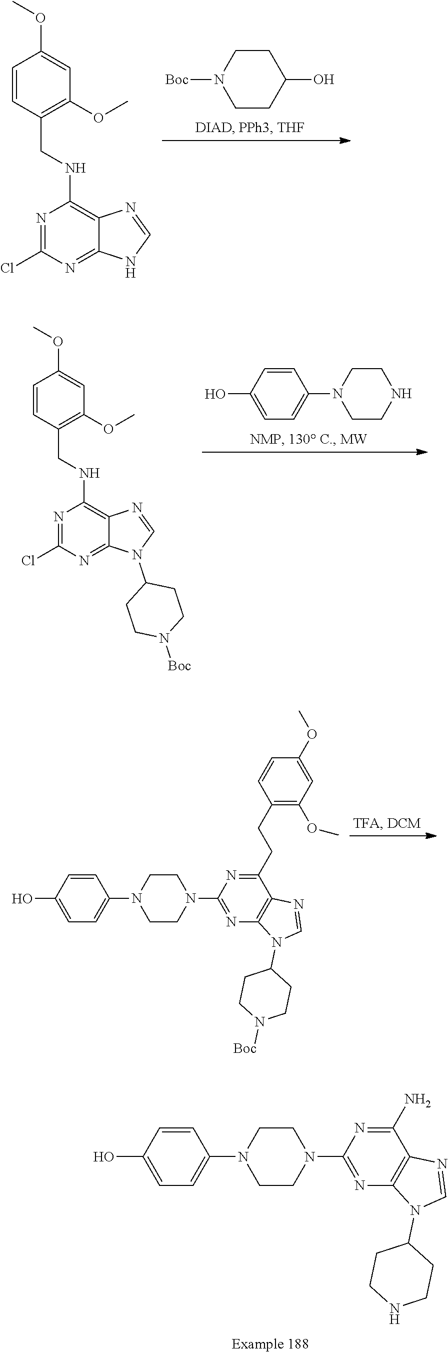 [SMILES: COc1ccc(CNc2nc(Cl)nc3[nH]cnc23)c(OC)c1 ; CC(C)(C)OC(=O)N1CCC(O)CC1 ; COc1ccc(CNc2nc(Cl)nc3c2ncn3C2CCN(C(=O)OC(C)(C)C)CC2)c(OC)c1 ; Oc1ccc(N2CCNCC2)cc1 ; COc1ccc(CCc2nc(N3CCN(c4ccc(O)cc4)CC3)nc3c2ncn3C2CCN(C(=O)OC(C)(C)C)CC2)c(OC)c1 ; Nc1nc(N2CCN(c3ccc(O)cc3)CC2)nc2c1ncn2C1CCNCC1]

### Step 1

A mixture of 2-chloro-N-(2,4-dimethoxybenzyl)-9H-purin-6-amine (200 mg, 0.63 mmol, 1.0 eq), triphenyl phosphine (250 mg, 0.94 mmol, 1.5 eq), DIAD (190 mg, 0.94 mmol, 1.5 eq) and tert-butyl 4-hydroxypipeidine-1-carboxylate (127 mg, 0.63 mmol, 1.0 eq) in THF (5 mL) was stirred at room temperature for 3 hours. The reaction mixture was evaporated to dryness in vacuum. The residue was purified with silica gel chromatography (EA) to give tert-butyl 4-(2-chloro-6-((2,4-dimethoxybenzyl)amino)-9H-purin-9-yl)piperidine-1-carboxylate (150 mg, 52% yield) as a white solid.

### Step 2

A mixture of tert-butyl 4-(2-chloro-6-((2,4-dimethoxybenzyl)amino)-9H-purin-9-yl)piperidine-1-carboxylate (130 mg, 0.26 mmol, 1.0 eq) and 4-(piperazin-1-yl) phenol (116 mg, 0.65 mmol, 2.5 eq) in NMP (3 mL) in a sealed vial was irradiated in the microwave at 130° C. for 1 h. After cooling, the reaction mixture was quenched with water (20 mL). The organic layer was separated and was concentrated to give tert-butyl 4-(6-((2,4-dimethoxybenzyl)amino)-2-(4-(4-hydroxyphenyl)piperazin-1-yl)-9H-purin-9-yl)piperidine-1-carboxylate (130 mg, 78% yield) as a brown solid.

### Step 3

A mixture of tert-butyl 4-(6-((2,4-dimethoxybenzyl)amino)-2-(4-(4-hydroxyphenyl)piperazin-1-yl)-9H-purin-9-yl)piperidine-1-carboxylate (130 mg, 0.20 mmol, 1 eq) in trifluoroacetic acid (1 mL) and dichloromethane (1 mL) was stirred at room temperature for 2 hours. The mixture was evaporated and the residue was purified by preparative HPLC to give 4-(4-(6-amino-9-(piperidin-4-yl)-9H-purin-2-yl)piperazin-1-yl)phenol (15 mg, 19% yield) as a white solid.

¹H NMR (400 MHz, DMSO-d6): δ 8.85 (s, 1H), 7.82 (s, 1H), 6.85-6.79 (m, 4H), 6.67 (d, J=9.2 Hz, 2H), 4.31-4.19 (m, 1H), 3.83-3.76 (m, 4H), 3.14-3.07 (m, 2H), 3.06-1.96 (m, 4H), 2.73-2.64 (m, 2H), 2.07-1.85 (m, 4H); LC/MS(ESI-MS): [M+H]+=395.3.

### Example 189. 3-(6-amino-2-(4-(4-hydroxyphenyl)piperazin-1-yl)-9H-purin-9-yl)cyclohexane-1-carboxylic acid

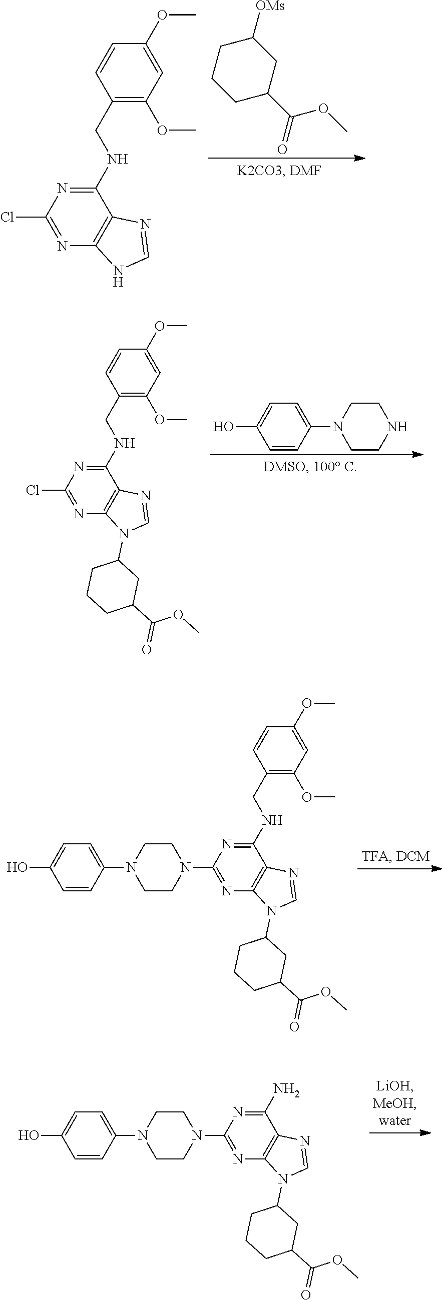 [SMILES: COc1ccc(CNc2nc(Cl)nc3[nH]cnc23)c(OC)c1 ; COC(=O)C1CCCC(OS(C)(=O)=O)C1 ; COC(=O)C1CCCC(n2cnc3c(NCc4ccc(OC)cc4OC)nc(Cl)nc32)C1 ; Oc1ccc(N2CCNCC2)cc1 ; COC(=O)C1CCCC(n2cnc3c(NCc4ccc(OC)cc4OC)nc(N4CCN(c5ccc(O)cc5)CC4)nc32)C1 ; COC(=O)C1CCCC(n2cnc3c(N)nc(N4CCN(c5ccc(O)cc5)CC4)nc32)C1]

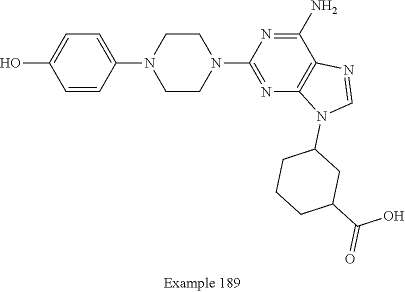 [SMILES: Nc1nc(N2CCN(c3ccc(O)cc3)CC2)nc2c1ncn2C1CCCC(C(=O)O)C1]

### Step 1

To a solution of 4 methyl 3-((methylsulfonyl)oxy)cyclohexane-1-carboxylate (700 mg, 2.96 mmol, 1.5 eq) in DMF (10 mL), 2-chloro-N-(2,4-dimethoxybenzyl)-9H-purin-6-amine (632 mg, 1.98 mmol, 1.0 eq), potassium carbonate (818 mg, 5.93 mmol, 3.0 eq) were added. The mixture was heated to 80° C. for 15 hours. The mixture was cooled to room temperature and diluted with water (20 mL). The reaction mixture was extracted with ethyl acetate (50 mL×3). The combined organic layers were washed with brine, dried over Na₂SO₄, filtered and concentrated under vacuum. The residue was purified by silica gel column chromatography to give methyl 3-(6-((2,4-dimethoxybenzyl)amino)-2-(4-(4-hydroxyphenyl)piperazin-1-yl)-9H-purin-9-yl)cyclohexane-1-carboxylate (300 mg, 33% yield) as a white solid.

### Step 2

To a solution of methyl 3-(6-((2,4-dimethoxybenzyl)amino)-2-(4-(4-hydroxyphenyl)piperazin-1-yl)-9H-purin-9-yl)cyclohexane-1-carboxylate (300 mg, 0.65 mmol, 1.0 eq) in DMSO (5 mL), 4-(piperazin-1-yl)phenol (291 mg, 1.63 mmol, 2.5 eq) was added. The mixture was heated to 100° C. for 12 hours. The mixture was cooled to the room temperature and was added water (20 mL) to afford a solid as the crude product. The crude was purified by preparative HPLC to give methyl 3-(6-((2,4-dimethoxybenzyl)amino)-2-(4-(4-hydroxyphenyl)piperazin-1-yl)-9H-purin-9-yl)cyclohexane-1-carboxylate (400 mg, 60% yield) as a black solid.

¹H NMR (400 MHz, DMSO-d6): δ 8.84 (s, 1H), 7.86 (s, 1H), 7.61-7.48 (m, 1H), 7.14 (d, J=8.4 Hz, 1H), 6.83 (d, J=8.8 Hz, 2H), 6.66 (d, J=9.2 Hz, 2H), 6.54-6.53 (m, 1H), 6.44-6.41 (m, 1H), 4.58-4.37 (m, 3H), 3.81-3.68 (m, 12H), 3.01-2.86 (m, 4H), 2.23-1.56 (m, 9H); LC/MS(ESI-MS): [M+H]+=602.3.

### Step 3

To a solution of methyl 3-(6-((2,4-dimethoxybenzyl)amino)-2-(4-(4-hydroxyphenyl)piperazin-1-yl)-9H-purin-9-yl)cyclohexane-1-carboxylate (700 mg, 1.16 mmol, 1 eq) in DCM (8 mL), TFA (3 mL) was added. The mixture was heated to 40° C. for 15 hours. The reaction mixture was concentrated to give, concentrated under vacuum to give methyl 3-(6-amino-2-(4-(4-hydroxyphenyl)piperazin-1-yl)-9H-purin-9-yl)cyclohexane-1-carboxylate (300 mg, 20% yield) as a black solid.

¹H NMR (400 MHz, DMSO-d6): δ 8.84 (s, 1H), 9.10 (br, 1H), 7.87-7.86 (m, J H), 6.86-6.79 (m, 4H), 6.67 (d, J=8.8 Hz, 2H), 4.43-4.26 (m, 1H), 3.68 (s, 2H), 3.61 (s, 2H), 3.01-2.95 (m, 4H), 2.23-1.56 (m, 8H); LC/MS(ESI-MS): [M+H]+=452.4.

### Step 4

To a solution of methyl 3-(6-amino-2-(4-(4-hydroxyphenyl)piperazin-1-yl)-9H-purin-9-yl)cyclohexane-1-carboxylate (300 mg, 0.66 mmol, 1 eq) in MeOH (5 mL) and water (5 mL), lithium hydroxide (140 mg, 3.32 mmol, 5 eq) was added. The mixture was stirred for 12 hours at room temperature. The reaction mixture was concentrated under vacuum. The pH of the mixture was adjusted to 3-4 by diluted aqueous HCl. Collected the crude product by filtration. The crude product was further purified by preparative HPLC to give 3-(6-amino-2-(4-(4-hydroxyphenyl)piperazin-1-yl)-9H-purin-9-yl)cyclohexane-1-carboxylic acid (120 mg, 41% yield) as a gray solid as a mixture of cis and trans isomers.

¹H NMR (400 MHz, DMSO-d6): δ 9.10 (br, 1H), 8.01 (s, 1H), 7.97 (s, 1H), 7.85 (s, 1H), 7.80 (s, 1H), 7.19-7.12 (m, 4H), 6.84 (d, J=9.2 Hz, 2H), 6.77-6.74 (m, 2H), 6.68 (d, J=9.2 Hz, 2H), 4.65-4.49 (m, 2H), 4.22-4.13 (m, 2H), 3.81-3.73 (m, 12H), 3.01-2.95 (m, 6H), 2.43-2.21 (m, 2H), 2.11-1.34 (m, 12H); LC/MS(ESI-MS): [M+H]+=438.2.

### Example 190. 2-(4-(5-aminopyridin-2-yl)piperazin-1-yl)-9-(pyridin-4-yl)-9H-purin-6-amine

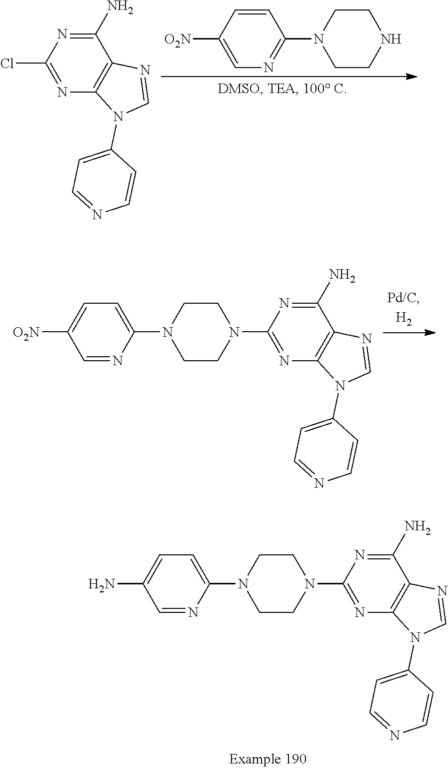 [SMILES: Nc1nc(Cl)nc2c1ncn2-c1ccncc1 ; O=[N+]([O-])c1ccc(N2CCNCC2)nc1 ; Nc1nc(N2CCN(c3ccc([N+](=O)[O-])cn3)CC2)nc2c1ncn2-c1ccncc1 ; Nc1ccc(N2CCN(c3nc(N)c4ncn(-c5ccncc5)c4n3)CC2)nc1]

### Step 1

To a solution of 2-chloro-9-(pyridin-4-yl)-9H-purin-6-amine (200 mg, 0.81 mmol, 1.0 eq) in DMSO (5 mL), 1-(5-nitropyridin-2-yl)piperazine (423 mg, 2.08 mmol, 2.5 eq), triethylamine (246 mg, 2.43 mmol, 3 eq) were added. The mixture was stirred at 100° C. for 15 hours. The mixture was cooled to room temperature and diluted with water (20 mL) to give a solid. Collected the solid by filtration to give 2-(4-(5-nitropyridin-2-yl)piperazin-1-yl)-9-(pyridin-4-yl)-9H-purin-6-amine (200 mg, 59/a yield) as a black solid.

### Step 2

To a solution of 2-(4-(5-nitropyridin-2-yl)piperazin-1-yl)-9-(pyridin-4-yl)-9H-purin-6-amine (200 mg, 0.478 mmol, 1 eq) in MeOH (5 mL), was added palladium on carbon (20%, 40 mg). The reaction mixture was stirred under a balloon pressure of hydrogen at 50° C. for 12 hours. The reaction was cooled at room temperature and the filtrate was concentrated under reduced pressure. The residue was purified by preparative HPLC to give 2-(4-(5-aminopyridin-2-yl)piperazin-1-yl)-9-(pyridin-4-yl)-9H-purin-6-amine (9 mg, 5% yield) as a gray solid.

¹H NMR (400 MHz, DMSO-d6): δ 8.81 (d, J=6.0 Hz, 2H), 8.62 (s, 1H), 8.37 (d, J=6.0 Hz, 2H), 7.83 (br, 1H), 7.56-7.53 (m, 1H), 7.25-7.11 (m, 3H), 3.89-3.86 (m, 4H), 3.61-3.59 (m, 4H); LC/MS(ESI-MS): [M+H]+=389.2.

### Example 191. 2-(4-(4-(6-amino-9-(pyridin-4-yl)-9H-purin-2-yl)piperazin-1-yl)phenyl)ethan-1-ol

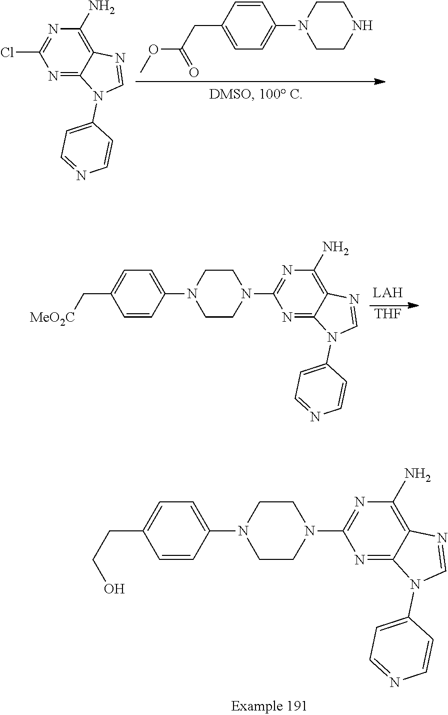

### Step 1

To a solution of methyl 2-(4-(piperazin-1-yl)phenyl)acetate (101 mg, 0.45 mol, 1.0 eq) in DMSO (3 mL), 2-chloro-9-(pyridin-4-yl)-9H-purin-6-amine (45.1 mg, 0.18 mmol, 0.4 eq), DIEA (177.2 mg, 1.37 mmol, 3 eq) were added. The mixture was heated at 100° C. for 15 hours. The mixture was cooled and diluted with water (10 mL) to afford a solid as a crude product. The crude was further purified by silica-gel chromography (MeOH:DCM=6:94, v/v) to get methyl 2-(4-(4-(6-amino-9-(pyridin-4-yl)-9H-purin-2-yl)piperazin-1-yl)phenyl)acetate (75 mg, 50% yield) as a gray solid.

### Step 2

To a solution of methyl 2-(4-(4-(6-amino-9-(pyridin-4-yl)-9H-purin-2-yl)piperazin-1-yl)phenyl)acetate (76 mg, 0.17 mmol, 1.0 eq) in THF (5 mL), LAH (13.4 mg, 0.35 mmol, 2 eq) was added at 0° C. under N2. The reaction mixture was stirred at room temperature for 15 hours. The mixture was diluted with aqueous NaOH at 0° C. to obtain a solid. The solid was filtrated and the mixture was concentrated under reduced pressure. The residue was purified by preparative HPLC to get 2-(4-(4-(6-amino-9-(pyridin-4-yl)-9H-purin-2-yl)piperazin-1-yl)phenyl)ethan-1-ol (2 mg, 5% yield) as a white solid.

¹H NMR (400 MHz, DMSO-d6): δ8.82 (d, J=6.0 Hz, 2H), 8.61 (s, 1H), 8.41 (d, J=5.6 Hz, 2H), 7.22 (br, 2H), 7.10 (d, J=8.4 Hz, 2H), 6.95 (d, J=8.4 Hz, 2H), 4.92-4.85 (m, 4H), 3.56-3.52 (m, 2H), 3.21-3.13 (m, 3H), 2.72-2.56 (m, 4H); LC/MS(ESI-MS): [M+H]+=417.3.

### Example 192. (4-(4-(6-amino-9-(pyridin-4-yl)-9H-purin-2-yl)piperazin-1-yl)phenyl)methanol

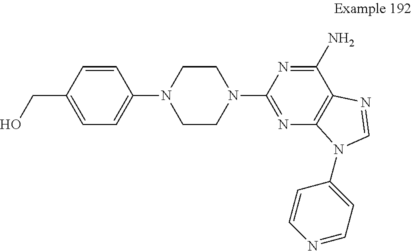 [SMILES: Nc1nc(N2CCN(c3ccc(CO)cc3)CC2)nc2c1ncn2-c1ccncc1]

The proceeding example was prepared according to Example 66 by substituting 4-(piperazin-1-yl)phenol with (4-(piperazin-1-yl)phenyl)methanol, phenyl boronic acid with 4 pyridin-4-ylboronic acid. The desired (4-(4-(6-amino-9-(pyridin-4-yl)-9H-purin-2-yl)piperazin-1-yl)phenyl)methanol was isolated as a white solid.

1HNMR (400 MHz, DMSO-d6): δ 8.72 (d, J=4.8 Hz, 2H), 8.54 (s, 1H), 8.19 (d, J=5.6 Hz, 2H), 7.19-7.10 (m, 4H), 6.97 (d, J=8.8 Hz, 2H), 4.97 (t, J=5.6 Hz, 1H), 4.39 (d, J=5.6 Hz, 2H), 3.94-3.86 (m, 4H), 3.21-3.14 (m, 4H); LC/MS(ESI-MS): [M+H]+=403.2.

### Example 193. 6-(4-(6-amino-9-(pyridin-4-yl)-9H-purin-2-yl)piperazin-1-yl)nicotinic acid

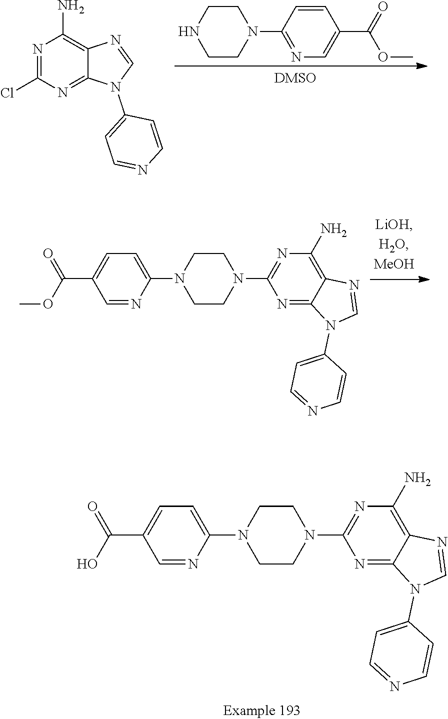 [SMILES: Nc1nc(Cl)nc2c1ncn2-c1ccncc1 ; COC(=O)c1ccc(N2CCNCC2)nc1 ; COC(=O)c1ccc(N2CCN(c3nc(N)c4ncn(-c5ccncc5)c4n3)CC2)nc1 ; Nc1nc(N2CCN(c3ccc(C(=O)O)cn3)CC2)nc2c1ncn2-c1ccncc1]

### Step 1

To a solution of methyl 6-(piperazin-1-yl)nicotinate (400 mg, 11.8 mmol, 2.5 eq) into DMSO (3 mL), 2-chloro-9-(pyridin-4-yl)-9H-purin-6-amine (179 mg, 0.72 mmol, 1 eq) was added. The mixture was heated at 100° C. overnight for 12 hours under an atmosphere of nitrogen. The mixture was diluted with water (5 mL) to afford a solid. Collect the solid by filtration to get methyl 6-(4-(6-amino-9-(pyridin-4-yl)-9H-purin-2-yl)piperazin-1-yl)nicotinate (600 mg, 100% yield) to use in the next step without further purification.

### Step 2

To a solution of methyl 6-(4-(6-amino-9-(pyridin-4-yl)-9H-purin-2-yl)piperazin-1-yl)nicotinate (600 mg, 1.39 mmol, 1.0 eq) into MeOH (5 mL) and water (2 mL), LiOH (291.8 mg, 6.95 mmol, 5 eq) was added. The mixture was stirred at room temperature for 2 hours. The solvent was removed under reduced pressure. Adjusted pH of the residue to 3-4 by aqueous HCl to afford a solid as a crude product. Purify the crude product by preparative HPLC to give 6-(4-(6-amino-9-(pyridin-4-yl)-9H-purin-2-yl)piperazin-1-yl)nicotinic acid (10 mg, 3% yield) as white solid.

¹H NMR (400 MHz, DMSO-d6): δ 8.72 (d, J=6.4 Hz, 2H), 8.64 (s, 1H), 8.55 (s, 1H), 8.20 (d, J=6.4 Hz, 2H), 8.00-7.97 (m, 1H), 7.14 (br, 2H), 6.78 (d, J=8.8 Hz, 1H), 3.91-3.82 (m, 4H), 3.76-3.61 (m, 4H); LC/MS(ESI-MS): [M+H]+=418.1.

### Example 194. 6-(4-(6-amino-9-(pyridin-4-yl)-9H-purin-2-yl)piperazin-1-yl)nicotinamide

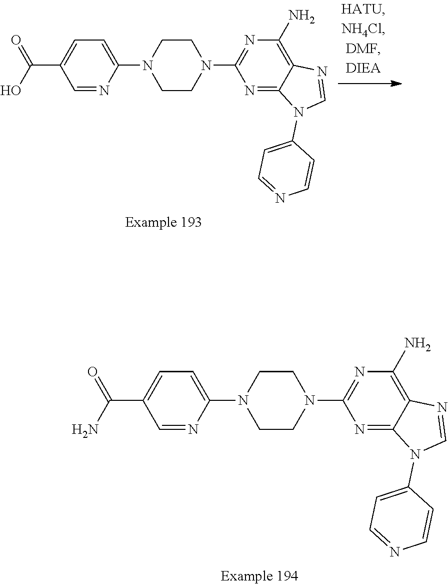 [SMILES: Nc1nc(N2CCN(c3ccc(C(=O)O)cn3)CC2)nc2c1ncn2-c1ccncc1 ; NC(=O)c1ccc(N2CCN(c3nc(N)c4ncn(-c5ccncc5)c4n3)CC2)nc1]

To a solution of 6-(4-(6-amino-9-(pyridin-4-yl)-9H-purin-2-yl)piperazin-1-yl)nicotinic acid (100 mg, 0.24 mmol, 1.0 eq) into DMF (5 mL), HATU (91 mg, 0.24 mmol, 1 eq) and DIEA (92.9 mg, 0.72 mmol, 3 eq) were added. The reaction was allowed to stir for 30 minutes at room temperature, NH4Cl (19.2 mg, 0.36 mmol, 1.5 eq) were added and the mixture was stirred at room temperature for 15 hours under N2. The mixture was diluted with water (5 mL) to obtain the solid as the crude product. The crude was further purified by preparative HPLC to get 6-(4-(6-amino-9-(pyridin-4-yl)-9H-purin-2-yl)piperazin-1-yl)nicotinamide (50 mg, 50% yield).

¹H NMR (400 MHz, DMSO-d6): δ 8.90-8.60 (m, 3H), 8.63-8.57 (m, 1H), 8.27-8.13 (m, 2H), 8.07-7.91 (m, 1H), 7.87-7.73 (m, 1H), 7.25-7.07 (m, 3H), 6.95-6.81 (m, 1H), 3.97-3.82 (m, 4H), 3.80-3.62 (m, 4H); LC/MS(ESI-MS): [M+H]+=417.2.

### Example 195. 1-(1-(6-amino-9-(pyridin-4-yl)-9H-purin-2-yl)piperidin-4-yl)-1H-pyrazole-4-carboxylic acid

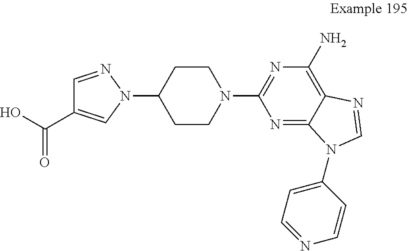 [SMILES: Nc1nc(N2CCC(n3cc(C(=O)O)cn3)CC2)nc2c1ncn2-c1ccncc1]

The proceeding example was prepared according to Example 193 by substituting methyl 6-(piperazin-1-yl)nicotinate with methyl 1-(piperidin-4-yl)-1H-pyrazole-4-carboxylate. The desired 1-(1-(6-amino-9-(pyridin-4-yl)-9H-purin-2-yl)piperidin-4-yl)-1H-pyrazole-4-carboxylic acid was isolated as a white solid.

¹H NMR (400 MHz, DMSO-d6): δ 12.27 (br, 1H), 8.71 (d, J=6.0 Hz, 2H), 8.52 (s, 1H), 8.31 (s, 1H), 8.17 (d, J=6.4 Hz, 2H), 7.80 (s, 1H), 7.10 (br, 2H), 4.82-4.78 (m, 2H), 4.53-4.47 (m, 1H), 3.05-2.88 (m, 2H), 2.10-2.07 (m, 2H), 1.94-1.87 (m, 2H); LC/MS(ESI-MS): [M+H]+=406.2.

### Example 196. (6-(4-(6-amino-9-(pyridin-4-yl)-9H-purin-2-yl)piperazin-1-yl)pyridin-3-yl)methanol

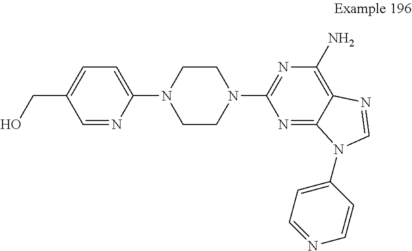 [SMILES: Nc1nc(N2CCN(c3ccc(CO)cn3)CC2)nc2c1ncn2-c1ccncc1]

The proceeding example was prepared according to Example 66 by substituting 4-(piperazin-1-yl)phenol with (6-(piperazin-1-yl)pyridin-3-yl)methanol, phenyl boronic acid with 4 pyridin-4-ylboronic acid. The desired (6-(4-(6-amino-9-(pyridin-4-yl)-9H-purin-2-yl)piperazin-1-yl)pyridin-3-yl)methanol was isolated as a white solid.

¹H NMR (400 MHz, DMSO-d6): δ 8.80-8.71 (m, 2H), 8.53 (s, 1H), 8.19 (d, J=5.6 Hz, 2H), 8.08 (s, 1H), 7.54-7.51 (m, 1H), 7.14 (br, 2H), 6.88 (d, J=8.8 Hz, 1H), 5.02 (t, J=5.2 Hz, 1H), 4.37 (d, J=5.2 Hz, 2H), 3.91-3.73 (m, 1H), 3.63-3.49 (m, 4H); LC/MS(ESI-MS): [M+H]+=404.2.

### Example 197. 1-(6-amino-9-(pyridin-4-yl)-9H-purin-2-yl)-4-phenylpiperidin-4-ol

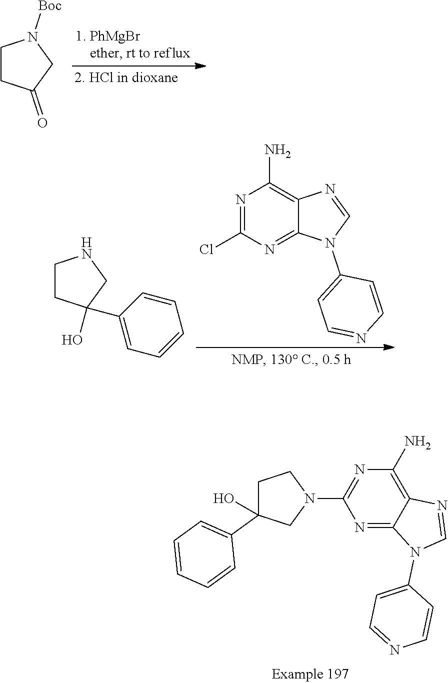 [SMILES: CC(C)(C)OC(=O)N1CCC(=O)C1 ; OC1(c2ccccc2)CCNC1 ; Nc1nc(Cl)nc2c1ncn2-c1ccncc1 ; Nc1nc(N2CCC(O)(c3ccccc3)C2)nc2c1ncn2-c1ccncc1]

### Step 1

To a solution of tert-butyl 3-oxopyrolidine-1-carboxylate (1.0 g, 5.4 mmol, 1.0 eq) in Et₂O (10 mL) at −20° C. under N2, phenylmagnesium bromide (6.4 ml, 6.4 mmol, 1.2 eq) was added. The reaction mixture was stirred at room temperature for 2 hours. The reaction mixture was evaporated to dryness in vacuum. The residue was purified with silica gel chromatography (PE:EA=5:1) to give the intermediate tert-butyl 3-hydroxy-3-phenylpyrrolidine-1-carboxylate (560 mg, 39% yield) as a white solid. Added HCl in dioxane (2.2 ml, 8.73 mmol, 5.0 eq) to a mixture of tert-butyl 3-hydroxy-3-phenylpyrrolidine-1-carboxylate (460 mg, 1.75 mmol, 1.0 eq) in dioxane (3 mL). The reaction mixture was stirred at room temperature for 2 hours. The reaction mixture was evaporated to dryness in vacuum to give 3-phenylpyrrolidin-3-ol (240 mg, 83%) as a pink solid.

### Step 2

A mixture of 3-phenylpyrrolidin-3-ol (242 mg, 1.22 mmol, 2.5 eq), 2-chloro-9-(pyridin-4-yl)-9H-purin-6-amine (120 mg, 0.49 mmol, 1.0 eq) and DIEA (190 mg, 1.47 mmol, 3.0 eq) in 3 ml of NMP in a sealed vial was heated in the microwave at 130° C. for 0.5 h. The reaction was quenched with water (20 mL) and the mixture was concentrated under reduced pressure to give 1-(6-amino-9-(pyridin-4-yl)-9H-purin-2-yl)-4-phenylpiperidin-4-ol (140 mg, 76%) as a white solid. 1HNMR (400 MHz, DMSO-d6): δ 8.69 (s, 2H), 8.52 (s, 1H), 8.30-8.13 (m, 2H), 7.64-7.58 (m, 2H), 7.40-7.36 (m, 2H), 7.29-7.26 (m, 1H), 7.10-6.93 (m, 2H), 5.44 (s, 1H), 3.92-3.88 (m, 1H), 3.84-3.80 (m, 1H), 3.70-3.67 (m, 2H), 2.35-2.21 (m, 1H), 2.19-2.01 (m, 1H); LC/MS(ESI-MS): [M+H]+=406.2.

### Example 198. 1-(6-amino-9-(pyridin-4-yl)-9H-purin-2-yl)-4-(3-hydroxyprop-1-yn-1-yl)piperidin-4-ol

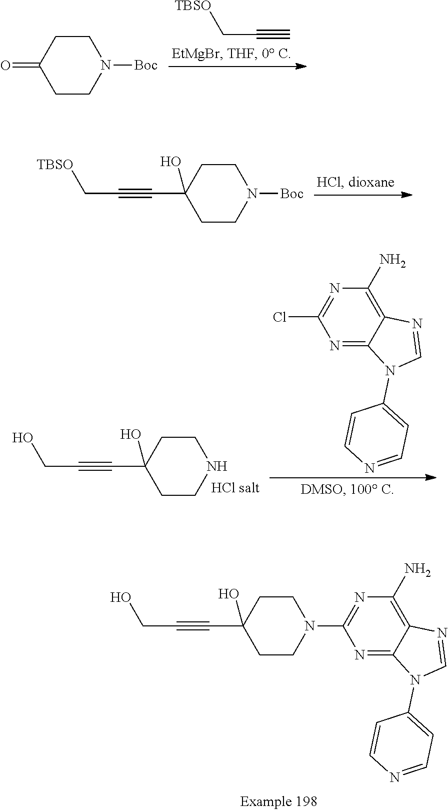 [SMILES: C#CCO[Si](C)(C)C(C)(C)C ; CC(C)(C)OC(=O)N1CCC(=O)CC1 ; CC(C)(C)OC(=O)N1CCC(O)(C#CCO[Si](C)(C)C(C)(C)C)CC1 ; OCC#CC1(O)CCNCC1 ; Nc1nc(Cl)nc2c1ncn2-c1ccncc1 ; Nc1nc(N2CCC(O)(C#CCO)CC2)nc2c1ncn2-c1ccncc1]

### Step 1

To a solution of tert-butyl 4-oxopiperidine-1-carboxylate (1.99 g, 0.01 mol, 1.0 eq) in THF (10 mL), tert-butyldimethyl(prop-2-yn-1-yloxy)silane (1.7 g, 0.01 mol, 1.0 eq) was added. The mixture was cooled to 0° C., EtMgBr (10 mmol, 1.0 eq) was added and the reaction mixture was stirred at room temperature for 2 hours. Water (20 mL) was added and the mixture was extracted by ethyl acetate (30 mL×2). The combined organic layers were evaporated to dryness in vacuum to obtain tert-butyl 4-(3-(tert-butyldimethylsilyl)oxy)prop-1-yn-1-yl)-4-hydroxypiperidine-1-carboxylate (1.1 g, 30% yield) as a white solid.

### Step 2

To a solution of tert-butyl 4-(3-((tert-butyldimethylsilyl)oxy)prop-1-yn-1-yl)-4-hydroxypiperidine-1-carboxylate (1.1 g, 2.98 mmol, 1.0 eq) in dioxane (10 mL), added HCl in dioxane (10 mL) and stirred at room temperature for 2 hours. The mixture was filtered and collect the solid to give 4-(3-hydroxyprop-1-yn-1-yl)piperidin-4-ol hydrogen chloride (1.1 g, 76%) as a white solid to used in the next step without further purification.

### Step 3

To a solution of 4-(3-hydroxyprop-1-yn-1-yl)piperidin-4-ol hydrogen chloride (350 mg, 2.26 mmol, 2.5 eq) in 5 mL of DMSO, 2-chloro-9-(pyridin-4-yl)-9H-purin-6-amine (222 mg, 0.9 mmol, 1.0 eq) was added and the mixture was stirred at 100° C. for 15 hours. Water (20 mL) was added and to obtain a precipitate. The white solid was purified with preparative HPLC to get 1-(6-amino-9-(pyridin-4-yl)-9H-purin-2-yl)-4-(3-hydroxyprop-1-yn-1-yl)piperidin-4-ol (5 mg, 0.6% yield).

¹H NMR (400 MHz, DMSO-d6): δ 8.80-8.63 (m, 2H), 8.51 (s, 1H), 8.20-8.03 (m, 2H), 7.11-6.92 (m, 2H), 5.51 (s, 1H), 5.15 (s, 1H), 4.17-3.99 (m, 4H), 3.57-3.51 (m, 2H), 1.77-1.69 (m, 2H), 1.70-1.59 (m, 2H). LCMS(ESI-MS): [M+H]+=366.1.

### Example 199. 1-(6-amino-9-(pyridin-4-yl)-9H-purin-2-yl)-3-(1H-pyrazol-4-yl)azetidin-3-ol

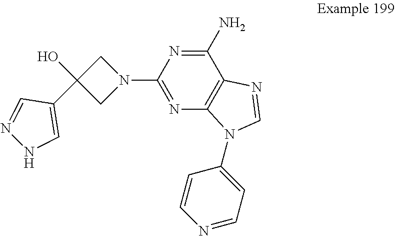 [SMILES: Nc1nc(N2CC(O)(c3cn[nH]c3)C2)nc2c1ncn2-c1ccncc1]

The proceeding example was prepared according to Example 197 by substituting 3-phenylpyrrolidin-3-ol with 3-(1H-pyrazol-4-yl)azetidin-3-ol. The desired 1-(6-amino-9-(pyridin-4-yl)-9H-purin-2-yl)-3-(1H-pyrazol-4-yl)azetidin-3-ol was isolated as a brown solid.

¹H NMR (400 MHz, DMSO-d6): δ8.92 (d, J=6.4 Hz, 2H), 8.74 (s, 1H), 8.63 (d, J=6.4 Hz, 2H), 7.79-7.62 (m, 3H), 4.27-4.18 (m, 4H); LC/MS(ESI-MS): [M+H]+=350.1.

### Example 200. 3-(1H-pyrazol-4-yl)-1-(9-(pyridin-4-yl)-9H-purin-2-yl)azetidin-3-ol

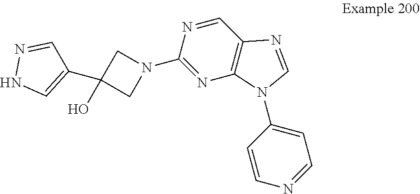 [SMILES: OC1(c2cn[nH]c2)CN(c2ncc3ncn(-c4ccncc4)c3n2)C1]

The proceeding example was prepared according to Example 199 by substituting 2-chloro-9-(pyridin-4-yl)-9H-purin-6-amine with 2-chloro-9-(pyridin-4-yl)-9H-purine. The desired 3-(1H-pyrazol-4-yl)-1-(9-(pyridin-4-yl)-9H-purin-2-yl)azetidin-3-ol was isolated as a brown solid.

¹H NMR (400 MHz, DMSO-d6): δ 12.71 (s, 1H), 8.87 (s, 2H), 8.75 (d, J=6.0 Hz, 2H), 8.19 (d, J=6.4 Hz, 2H), 7.68 (br, 2H), 6.12 (s, 1H), 4.32-4.24 (m, 4H); LC/MS(ESI-MS): [M+H]+=335.1.

### Example 201. 1-(6-amino-9-(pyridin-4-yl)-9H-purin-2-yl)-3-(thiazol-2-yl)azetidin-3-ol

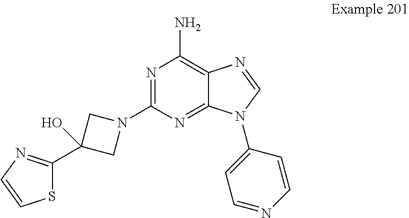 [SMILES: Nc1nc(N2CC(O)(c3nccs3)C2)nc2c1ncn2-c1ccncc1]

The proceeding example was prepared according to Example 193 by substituting 3-phenylpyrrolidin-3-ol with 3-(thiazol-2-yl)azetidin-3-ol. The desired 1-(6-amino-9-(pyridin-4-yl)-9H-purin-2-yl)-3-(thiazol-2-yl)azetidin-3-ol was isolated as a white solid.

¹H NMR (400 MHz, DMSO-d6): δ 8.70-8.68 (m, 2H), 8.56 (s, 1H), 8.21-8.20 (m, 2H), 7.79 (d, J=3.2 Hz, 1H), 7.69 (d, J=3.2 Hz, 1H), 7.22 (br, 3H), 4.44 (d, J=8.8 Hz, 2H), 4.22 (d, J=8.8 Hz, 2H); LC/MS(ESI-MS): [M+H]+=367.1.

### Example 202. 1-(6-amino-9-(pyridin-4-yl)-9H-purin-2-yl)-4-phenylpiperidin-4-ol

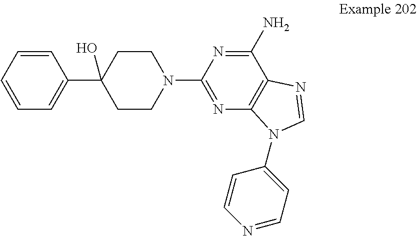 [SMILES: Nc1nc(N2CCC(O)(c3ccccc3)CC2)nc2c1ncn2-c1ccncc1]

The proceeding example was prepared according to Example 66 by substituting 4-(piperazin-1-yl)phenol with 4-phenylpiperidin-4-ol, phenyl boronic acid with 4 pyridin-4-ylboronic acid. The desired 1-(6-amino-9-(pyridin-4-yl)-9H-purin-2-yl)-4-phenylpiperidin-4-ol was isolated as a brown solid.

¹H NMR (400 MHz, DMSO-d6): 8.71 (d, J=6.4 Hz, 2H), 8.51 (s, 1H), 8.19 (d, J=6.0 Hz, 2H), 7.49 (d, J=7.6 Hz, 2H), 7.33-7.28 (m, 2H), 7.20 (t, J=7.2 Hz, 1H), 7.11-6.95 (m, 2H), 5.01 (s, 1H), 4.64-4.61 (m, 2H), 3.31-3.27 (m, 2H), 1.94-1.87 (m, 2H), 1.69-1.66 (m, 2H); LC/MS(ESI-MS): [M+H]+=388.1.

### Example 203. 4-(4-hydroxyphenyl)-1-(6-methyl-9-(2-methylpyridin-4-yl)-9H-purin-2-yl)piperidin-4-ol

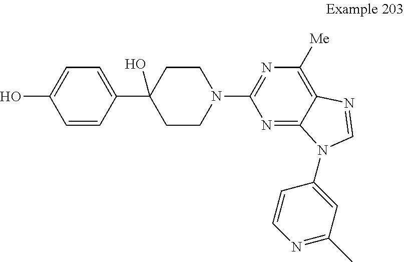 [SMILES: Cc1cc(-n2cnc3c(C)nc(N4CCC(O)(c5ccc(O)cc5)CC4)nc32)ccn1]

The proceeding example was prepared according to Example 43 by substituting 4-(piperazin-1-yl)phenol with 4-(4-hydroxyphenyl)piperidin-4-ol, phenyl boronic acid with 3-methylpyridin-4-ylboronic acid. The desired 4-(4-hydroxyphenyl)-1-(6-methyl-9-(2-methylpyridin-4-yl)-9H-purin-2-yl)piperidin-4-ol was isolated as a white solid.

¹H NMR (400 MHz, DMSO-d6): δ 9.19 (s, 1H), 8.72 (s, 2H), 8.60 (d, J=5.2 Hz, 1H), 8.03-7.98 (m, 2H), 7.27 (d, J=8.8 Hz, 2H), 6.69 (d, J=8.8 Hz, 2H), 4.93 (s, 1H), 4.65-4.55 (m, 2H), 3.37-3.31 (m, 2H), 2.59 (s, 3H), 2.56 (s, 3H), 1.96-1.83 (m, 2H), 1.77-1.68 (m, 2H); LC/MS(ESI-MS): [M+H]+=417.1.

### Example 204. 4-(4-hydroxyphenyl)-1-(6-methyl-9-(pyridin-3-yl)-9H-purin-2-yl)piperidin-4-ol

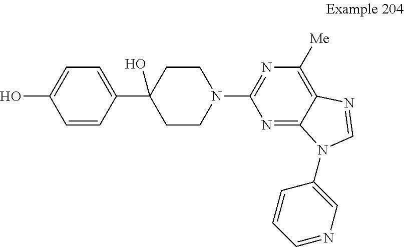 [SMILES: Cc1nc(N2CCC(O)(c3ccc(O)cc3)CC2)nc2c1ncn2-c1cccnc1]

The proceeding example was prepared according to Example 43 by substituting 4-(piperazin-1-yl)phenol with 4-(4-hydroxyphenyl)piperidin-4-ol, phenyl boronic acid with pyridin-3-ylboronic acid. The desired 4-(4-hydroxyphenyl)-1-(6-methyl-9-(2-methylpyridin-4-yl)-9H-purin-2-yl)piperidin-4-ol was isolated as a white solid.

¹H NMR (400 MHz, DMSO-d6): δ 9.19 (s, 1H), 8.72 (s, 2H), 8.60 (d, J=5.2 Hz, 1H), 8.03-7.98 (m, 2H), 7.27 (d, J=8.8 Hz, 2H), 6.69 (d, J=8.8 Hz, 2H), 4.93 (s, 1H), 4.65-4.55 (m, 2H), 3.37-3.31 (m, 2H), 2.59 (s, 3H), 2.56 (s, 3H), 1.96-1.83 (m, 2H), 1.77-1.68 (m, 2H); LC/MS(ESI-MS): [M+H]+=417.1.

### Example 205. N-(2-(4-hydroxy-4-(4-hydroxyphenyl)piperidin-1-yl)-9-(pyridin-4-yl)-9H-purin-6-yl)-N-methylglycine

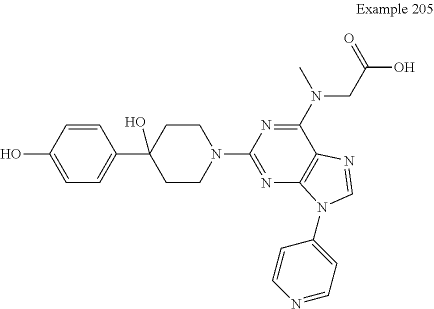 [SMILES: CN(CC(=O)O)c1nc(N2CCC(O)(c3ccc(O)cc3)CC2)nc2c1ncn2-c1ccncc1]

The proceeding example was prepared according to Example 163 by substituting methyl glycinate with methyl methylglycinate, 4-(piperazin-1-yl)phenol with 4-(4-hydroxyphenyl)piperidin-4-ol. N-(2-(4-hydroxy-4-(4-hydroxyphenyl)piperidin-1-yl)-9-(pyridin-4-yl)-9H-purin-6-yl)-N-methylglycine was isolated as a yellow solid.

¹H NMR (400 MHz, DMSO-d6): δ9.36 (br, 1H), 8.70-8.68 (m, 2H), 8.44-8.38 (m, 1H), 8.16-8.12 (m, 2H), 7.25 (d, J=7.2 Hz, 2H), 6.68 (d, J=8.4 Hz, 1H), 4.87 (s, 1H), 4.81-4.70 (m, 1H), 4.63-4.45 (m, 2H), 4.13-4.05 (m, 1H), 3.36 (s, 3H), 3.29-3.24 (m, 2H), 3.04-3.01 (m, 1H), 1.91-1.84 (m, 2H), 1.68-1.62 (m, 2H); LC/MS(ESI-MS): [M+H]+=476.2.

### Example 206. 4-(3-hydroxyphenyl)-1-(6-methyl-9-(pyridin-4-yl)-9H-purin-2-yl)piperidin-4-ol

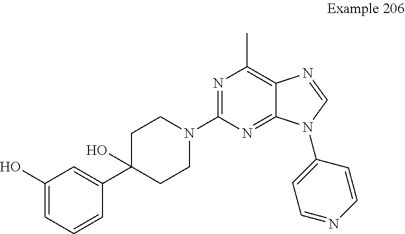 [SMILES: Cc1nc(N2CCC(O)(c3cccc(O)c3)CC2)nc2c1ncn2-c1ccncc1]

The proceeding example was prepared according to Example 43 by substituting 4-(piperazin-1-yl)phenol with 4-(3-hydroxyphenyl)piperidin-4-ol, phenyl boronic acid with pyridin-4-ylboronic acid. The desired -(3-hydroxyphenyl)-1-(6-methyl-9-(pyridin-4-yl)-9H-purin-2-yl)piperidin-4-ol was isolated as a white solid.

¹H NMR (400 MHz, DMSO-d6): δ9.18 (s, 1H), 8.73-8.70 (m, 3H), 8.15 (d, J=5.6 Hz, 2H), 7.05 (t, J=8.0 Hz, 1H), 6.89-6.82 (m, 2H), 6.57-6.55 (m, 1H), 5.02 (s, 1H), 4.65-4.59 (m, 2H), 3.41-3.32 (m, 2H), 2.57 (s, 3H), 1.85-1.82 (m, 2H), 1.68-1.65 (m, 2H); LC/MS(ESI-MS): [M+H]+=417.1.

### Example 207. (3S,4S)-1-(2-(4-hydroxy-4-(4-hydroxyphenyl)piperidin-1-yl)-9-(pyridin-4-yl)-9H-purin-6-yl)pyrrolidine-3,4-diol

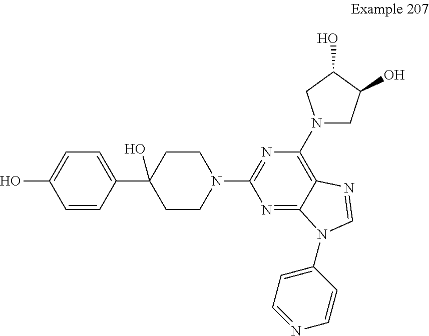 [SMILES: Oc1ccc(C2(O)CCN(c3nc(N4C[C@H](O)[C@@H](O)C4)c4ncn(-c5ccncc5)c4n3)CC2)cc1]

The proceeding example was prepared according to Example 163 by substituting methyl glycinate with (3S,4S)-pyrrolidine-3,4-diol, 4-(piperazin-1-yl)phenol with 4-(4-hydroxyphenyl)piperidin-4-ol. (3S,4S)-1-(2-(4-hydroxy-4-(4-hydroxyphenyl) piperidin-1-yl)-9-(pyridin-4-yl)-9H-purin-6-yl)pyrrolidine-3,4-diol was isolated as a white solid.

¹H NMR (400 MHz, DMSO-d6): δ 9.16 (s, 1H), 8.73-8.71 (m, 2H), 8.48 (s, 1H), 8.21-8.20 (m, 2H), 7.26 (d, J=8.8 Hz, 2H), 6.67 (d, J=8.4 Hz, 2H), 5.21-5.07 (m, 2H), 4.87 (s, 1H), 4.57-4.53 (m, 2H), 4.19-4.06 (m, 2H), 4.05-3.94 (m, 2H), 3.70-3.62 (m, 2H), 3.32-3.18 (m, 2H), 1.89-1.81 (m, 2H), 1.67-1.64 (m, 2H); LC/MS(ESI-MS): [M+H]+=489.9.

### Example 208. 1-(6-amino-9-(pyridin-3-yl)-9H-purin-2-yl)-4-(4-hydroxyphenyl)piperidin-4-ol

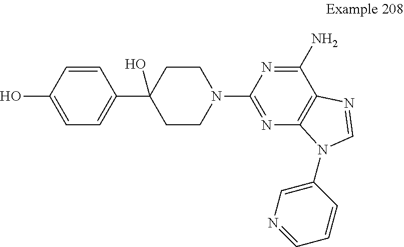 [SMILES: Nc1nc(N2CCC(O)(c3ccc(O)cc3)CC2)nc2c1ncn2-c1cccnc1]

The proceeding example was prepared according to Example 66 by substituting 4-(piperazin-1-yl)phenol with 4-(4-hydroxyphenyl)piperidin-4-ol, phenyl boronic acid with pyridin-3-ylboronic acid. The desired 1-(6-amino-9-(pyridin-3-yl)-9H-purin-2-yl)-4-(4-hydroxyphenyl)piperidin-4-ol was isolated as a white solid.

¹H NMR (400 MHz, DMSO-d6): δ 9.19 (s, 1H), 9.17 (s, 1H), 8.58-8.56 (m, 1H), 8.38-8.32 (m, 2H), 7.63-7.59 (m, 1H), 7.25 (d, J=8.4 Hz, 2H), 6.97 (br, 2H), 6.67 (d, J=8.8 Hz, 2H), 4.84 (s, 1H), 4.54-4.51 (m, 2H), 3.27-3.21 (m, 2H), 1.85-1.77 (m, 2H), 1.64-1.61 (m, 2H); LC/MS(ESI-MS): [M+H]+=404.2.

### Example 209. 1-(6-amino-9-(pyridin-3-yl)-9H-purin-2-yl)-4-(4-fluorophenyl)piperidin-4-ol

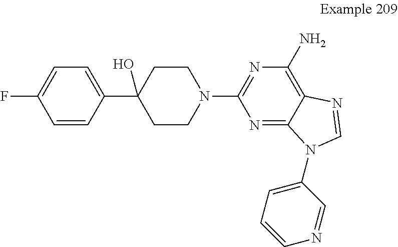 [SMILES: Nc1nc(N2CCC(O)(c3ccc(F)cc3)CC2)nc2c1ncn2-c1cccnc1]

The proceeding example was prepared according to Example 66 by substituting 4-(piperazin-1-yl)phenol with 4-(4-fluorophenyl)piperidin-4-ol, phenyl boronic acid with pyridin-3-ylboronic acid. The desired 1-(6-amino-9-(pyridin-3-yl)-9H-purin-2-yl)-4-(4-fluorophenyl)piperidin-4-ol was isolated as a white solid.

¹H NMR (400 MHz, DMSO-d6): δ 9.20 (s, 1H), 8.58-8.56 (m, 1H), 8.39-8.33 (m, 2H), 7.63-7.59 (m, 1H), 7.53-7.51 (m, 2H), 7.08-6.92 (m, 2H), 5.12 (s, 1H), 4.60-4.55 (m, 2H), 3.27-3.22 (m, 2H), 1.90-1.83 (m, 2H), 1.65 (d, J=12.8 Hz, 2H): LC/MS(ESI-MS): [M+H]+=406.2.

### Example 210. 1-(6-amino-9-(pyridin-4-yl)-9H-purin-2-yl)-4-(4-fluorophenyl)piperidin-4-ol

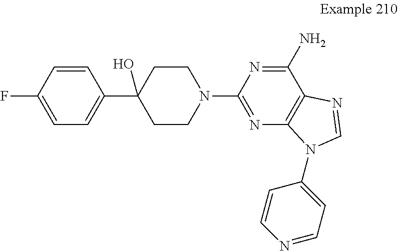 [SMILES: Nc1nc(N2CCC(O)(c3ccc(F)cc3)CC2)nc2c1ncn2-c1ccncc1]

The proceeding example was prepared according to Example 66 by substituting 4-(piperazin-1-yl)phenol with 4-(4-fluorophenyl)piperidin-4-ol, phenyl boronic acid with pyridin-4-ylboronic acid. The desired 1-(6-amino-9-(pyridin-4-yl)-9H-purin-2-yl)-4-(4-fluorophenyl)piperidin-4-ol was isolated as a white solid.

¹H NMR (400 MHz, DMSO-d6): δ 8.72-8.70 (m, 2H), 8.51 (s, 1H), 8.19-8.18 (m, 2H), 7.54-7.50 (m, 2H), 7.13-7.09 (m, 2H), 7.03 (s, 2H), 5.14 (s, 1H), 4.63-4.59 (m, 2H), 3.35-3.23 (m, 2H), 1.94-1.83 (m, 2H), 1.67 (d, J=9.2 Hz, 2H); LC/MS(ESI-MS): [M+H]+=406.2.

### Example 211. 1 4-(4-hydroxyphenyl)-1-(6-(piperidin-1-yl)-9-(pyridin-4-yl)-9H-purin-2-yl)piperidin-4-ol

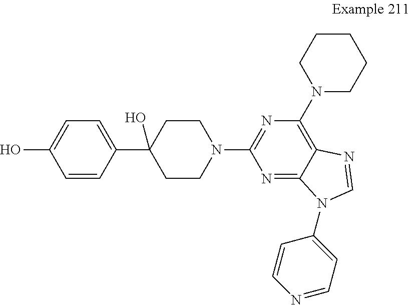 [SMILES: Oc1ccc(C2(O)CCN(c3nc(N4CCCCC4)c4ncn(-c5ccncc5)c4n3)CC2)cc1]

The proceeding example was prepared according to Example 163 by substituting methyl glycinate with piperidine, 4-(piperazin-1-yl)phenol with 4-(4-hydroxyphenyl) piperidin-4-ol. 1 4-(4-hydroxyphenyl)-1-(6-(piperidin-1-yl)-9-(pyridin-4-yl)-9H-purin-2-yl)piperidin-4-ol was isolated as a white solid.

¹H NMR (400 MHz, DMSO-d6): δ 8.88-8.73 (m, 3H), 8.55 (s, 1H), 8.12-7.98 (m, 2H), 7.68-7.63 (m, 1H), 7.42-7.11 (m, 1H), 6.95-6.78 (m, 1H), 4.54-4.27 (m, 2H), 4.20-4.09 (m, 2H), 4.05-3.78 (m, 4H), 1.78-1.51 (m, 8H), 1.36-1.29 (m, 2H); LC/MS(ESI-MS): [M+H]+=472.1.

### Example 212. 4-(1-(6-amino-9-(pyridin-4-yl)-9H-purin-2-yl)-3-hydroxyazetidin-3-yl)benzamide

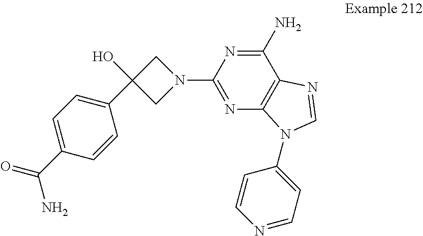 [SMILES: NC(=O)c1ccc(C2(O)CN(c3nc(N)c4ncn(-c5ccncc5)c4n3)C2)cc1]

The proceeding example was prepared according to Example 66 by substituting 4-(piperazin-1-yl)phenol with 4-(3-hydroxyazetidin-3-yl)benzamide, phenyl boronic acid with pyridin-4-ylboronic acid. The desired 4-(1-(6-amino-9-(pyridin-4-yl)-9H-purin-2-yl)-3-hydroxyazetidin-3-yl)benzamide was isolated as a white solid.

¹H NMR (400 MHz, DMSO-d6): δ8.75 (s, 2H), 8.61 (s, 1H), 8.36-8.29 (m, 2H), 7.98-7.88 (m, 3H), 7.31 (d, J=7.2 Hz, 2H), 7.31-7.19 (m, 3H), 4.25 (s, 4H); LC/MS(ESI-MS): [M−H]−=403.2.

### Example 213. 1-(6-amino-9-(pyridin-4-yl)-9H-purin-2-yl)-3-(isothiazol-5-yl)azetidin-3-ol

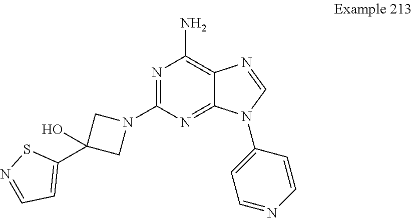 [SMILES: Nc1nc(N2CC(O)(c3ccns3)C2)nc2c1ncn2-c1ccncc1]

The proceeding example was prepared according to Example 66 by substituting 4-(piperazin-1-yl)phenol with 3-(isothiazol-5-yl)azetidin-3-ol, phenylboronic acid with pyridin-4-ylboronic acid. The desired 1-(6-amino-9-(pyridin-4-yl)-9H-purin-2-yl)-3-(isothiazol-5-yl)azetidin-3-ol was isolated as a white solid.

¹H NMR (400 MHz, DMSO-d6): δ 8.70-8.69 (m, 2H), 8.57 (s, 1H), 8.49 (d, J=1.6 Hz, 1H), 8.21-8.20 (m, 2H), 7.45 (d, J=1.6 Hz, 2H), 7.26 (s, 2H), 4.33-4.25 (m, 4H); LC/MS(ESI-MS): [M+H]+=367.1.

### Example 214. 1-(6-amino-9-(pyridin-4-yl)-9H-purin-2-yl)-3-(4-(hydroxymethyl)phenyl) azetidin-3-ol

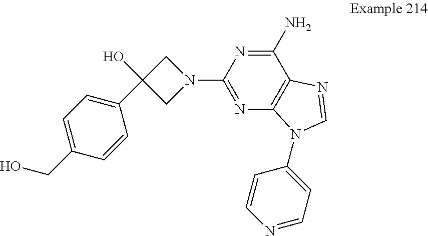 [SMILES: Nc1nc(N2CC(O)(c3ccc(CO)cc3)C2)nc2c1ncn2-c1ccncc1]

The proceeding example was prepared according to Example 66 by substituting 4-(piperazin-1-yl)phenol with 3-(4-(hydroxymethyl)phenyl)azetidin-3-ol, phenylboronic acid with pyridin-4-ylboronic acid. The desired 1-(6-amino-9-(pyridin-4-yl)-9H-purin-2-yl)-3-(4-(hydroxymethyl)phenyl)azetidin-3-ol was isolated as a white solid.

¹H NMR (400 MHz, DMSO-d6): δ8.77-8.76 (m, 2H), 8.61 (s, 1H), 8.38-8.32 (m, 2H), 7.51 (d, J=8.4 Hz, 2H), 7.33-7.30 (m, 4H), 4.49 (s, 2H), 4.24 (s, 4H); LC/MS(ESI-MS): [M+H]+=390.1.

### Example 215. 2-(2-(4-hydroxy-4-(4-hydroxyphenyl)piperidin-1-yl)-6-methyl-9H-purin-9-yl)acetic acid

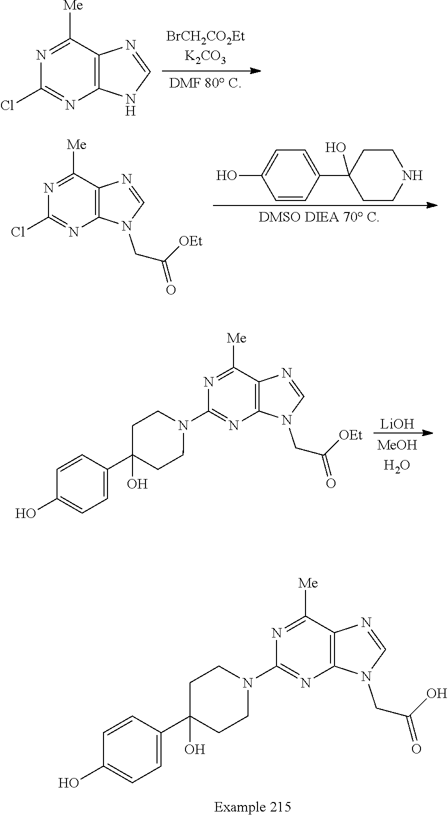

### Step 1

To a solution of 2-chloro-6-methyl-9H-purine (300 mg, 1.78 mmol, 1 eq) in DMF (10 mL) were added ethyl bromoacetate (445.76 mg, 2.7 mmol, 1.5 eq) and K2CO3 (738 mg, 5.3 mmol, 3 eq). The reaction was heated at 80° C. for 15 hours. The reaction was cooled to room temperature. Water (15 mL) was added and the mixture was extracted with ethyl acetate (15 mL×3). The combined organic layers were washed with brine, dried with Na₂SO₄, filtered and concentrated. The residue was purified by silica column chromatography to afford ethyl 2-(2-chloro-6-methyl-9H-purin-9-yl)acetate (280 mg, 53%) as a white solid.

### Step 2

To a solution of ethyl 2-(2-chloro-6-methyl-9H-purin-9-yl)acetate (280 mg, 0.935 mmol, 1.0 eq) in DMSO (45 mL), 4-(4-hydroxyphenyl)piperidin-4-ol (452 mg, 2.3 mmol, 2.5 eq) and DIEA (362 mg, 2.8 mmol, 3 eq) was added. The mixture was heated to 70° C. for 2 hours. The mixture was cooled to room temperature and water (10 mL) was added to get the crude product as a solid. Filtered the solid and future purified the crude by silica gel column chromatography (DCM:MeOH=10:1, v/v) to get ethyl 2-(2-(4-hydroxy-4-(4-hydroxyphenyl)piperidin-1-yl)-6-methyl-9H-purin-9-yl)acetate (75 mg, 16.5%) as a white solid.

### Step 3

To a solution of ethyl 2-(2-(4-hydroxy-4-(4-hydroxyphenyl)piperidin-1-yl)-6-methyl-9H-purin-9-yl)acetate (75 mg, 0.182 mmol, 1.0 eq) in MeOH (3 mL), was added LiOH (38.24 mg, 0.911 mmol, 5 eq) and H₂O (1 mL), the mixture was stirred at room temperature for 2 hours. Water (5 mL) was added and the mixture was extracted with ethyl acetate (15 mL×3). The combined organic layers were washed with brine, dried with Na₂SO₄, filtered and concentrated. The residue was purified by preparative HPLC to get 2-(2-(4-hydroxy-4-(4-hydroxyphenyl)piperidin-1-yl)-6-methyl-9H-purin-9-yl)acetic acid (3 mg, 4.3% yield) as a white solid.

¹H NMR (400 MHz, DMSO-d6): δ 9.23 (s, 1H), 7.88 (s, 1H), 7.24 (d, J=8.4 Hz, 2H), 6.67 (d, J=8.4 Hz, 2H), 5.32 (s, 1H), 4.59 (d, J=12.8 Hz, 2H), 4.37 (s, 2H), 3.24-3.18 (m, 2H), 2.60 (s, 3H), 2.08-1.95 (m, 1H), 1.89-1.81 (m, 2H), 1.77-1.69 (m, 2H); LC/MS(ESI-MS): [M−H]−=384.2.

### Example 216. 2-(2-(3-hydroxy-3-(4-hydroxyphenyl)azetidin-1-yl)-6-(1H-indazol-1-yl)-9H-purin-9-yl)acetic acid

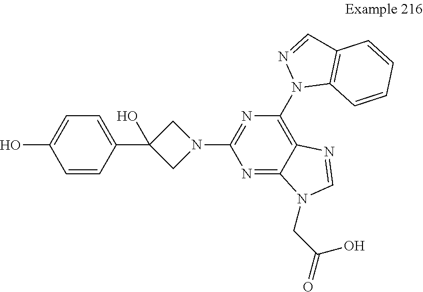 [SMILES: O=C(O)Cn1cnc2c(-n3ncc4ccccc43)nc(N3CC(O)(c4ccc(O)cc4)C3)nc21]

The proceeding example was prepared according to Example 21 by substituting 6-(piperazin-1-yl)pyridin-3-ol with 3-(4-hydroxyphenyl)azetidin-3-ol. The desired product 2-(2-(3-hydroxy-3-(4-hydroxyphenyl)azetidin-1-yl)-6-(1H-indazol-1-yl)-9H-purin-9-yl)acetic acid was isolated as a gray solid.

¹H NMR (400 MHz, DMSO-d6): δ 8.76 (d, J=8.4 Hz, 1H), 8.67-8.66 (m, 2H), 7.96 (d, J=8.0 Hz, 1H), 7.65 (t, J=7.6 Hz, 1H), 7.44-7.40 (m, 3H), 6.78 (d, J=8.4 Hz, 2H), 5.04 (s, 2H), 4.50-4.25 (m, 4H); LC/MS(ESI-MS): [M+H]+=458.2.

### Example 217. 5-(2-(4-hydroxy-4-(4-hydroxyphenyl)piperidin-1-yl)-6-(1H-indazol-1-yl)-9H-purin-9-yl)pentanoic acid

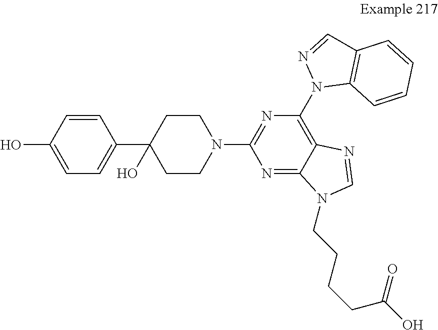 [SMILES: O=C(O)CCCCn1cnc2c(-n3ncc4ccccc43)nc(N3CCC(O)(c4ccc(O)cc4)CC3)nc21]

The proceeding example was prepared according to Example 21 by substituting ethyl 2-bromoacetate with methyl 5-bromovalerate, 6-(piperazin-1-yl)pyridin-3-ol with 3-(4-hydroxyphenyl)azetidin-3-ol. The desired product was isolated as a white solid.

1HNMR (400 MHz, DMSO-d6): δ 9.72 (br, 1H), 8.60-8.55 (m, 2H), 8.18 (s, 1H), 7.93 (d, J=8.0 Hz, 1H), 7.62-7.58 (m, 1H), 7.38-7.35 (m, 1H), 7.28 (d, J=8.4 Hz, 2H), 7.69 (d, J=8.4 Hz, 2H), 4.99 (s, 1H), 4.64-4.61 (m, 2H), 4.13 (t, J=6.4 Hz, 2H), 3.50-3.43 (m, 2H), 1.98-1.74 (m, 8H), 1.46-1.43 (m, 2H); LCMS(ESI-MS): [M+H]+=528.2.

### Example 218. 1-(9-(2-hydroxyethyl)-6-(1H-indazol-1-yl)-9H-purin-2-yl)-4-(4-hydroxyphenyl)piperidin-4-ol

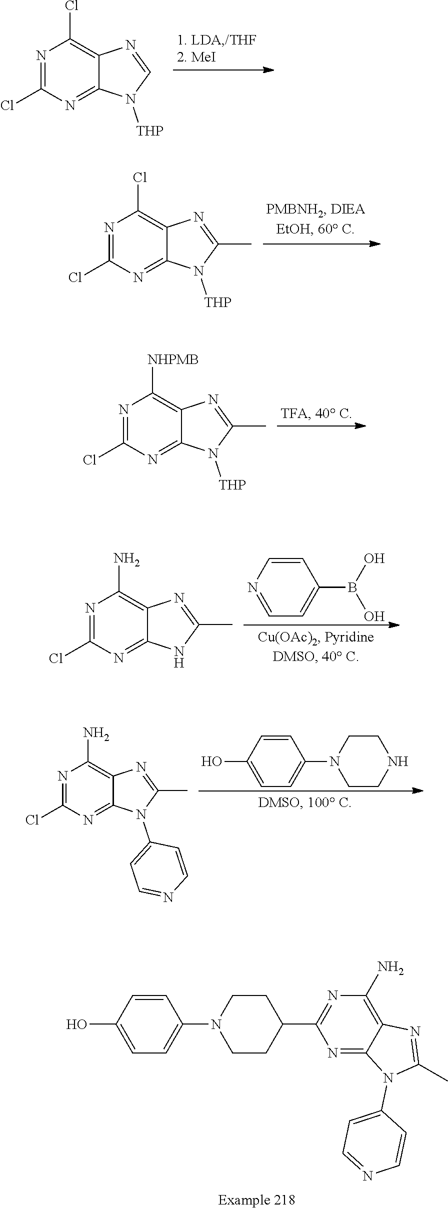

### Step 1

To a solution of 2,6-dichloro-9-(tetrahydro-2H-pyran-2-yl)-9H-purine (1.8 g, 6.6 mmol, 1.0 eq) in THF (20 mL) at −78° C., LDA (6.6 mL, 13 mmol, 2.0 eq) was added. The reaction was stirred at −78° C. for 30 minutes. Mel (0.82 mL, 13 mmol, 2.0 eq) was added. The reaction was stirred at room temperature for 15 hours. The reaction was quenched with H₂O at 0° C. The aqueous layer was extracted with ethyl acetate (3×50 mL). The combined organic layers were washed with water (3×25 mL), dried by Na₂SO₄, filtered and concentrated. The residue was purified using silica gel column chromatography (PE:EA=4:1) to get 2,6-dichloro-8-methyl-9-(tetrahydro-2H-pyran-2-yl)-9H-purine (1.20 g, 63% yield) as a yellow solid.

### Step 2

To a solution of (1.2 g, 3.55 mmol, 1.0 eq) in ethanol (30 mL) under N₂, DIEA (0.81 g, 6.27 mmol, 1.5 eq) and PMBNH2 (0.69 g, 5.02 mmol, 1.2 eq) were added. The reaction mixture was stirred at 60° C. for overnight. The reaction mixture was evaporated to dryness in vacuum and then the product was recrystallized from ethyl acetate to give 2-chloro-N-(4-methoxybenzyl)-8-methyl-9-(tetrahydro-2H-pyran-2-yl)-9H-purin-6-amine (1.58 g, 97% yield) as a yellow solid.

### Step 3

A mixture of 2-chloro-N-(4-methoxybenzyl)-8-methyl-9-(tetrahydro-2H-pyran-2-yl)-9H-purin-6-amine (1.58 g, 4.07 mmol, 1.0 eq) in trifluoroacetic acid (3 mL) was stirred at 40° C. for 15 hours. The reaction mixture was evaporated to dryness in vacuum. Adjusted the pH of the mixture to 9 using aqueous sodium carbonate solution to afford a solid. Collected the solid to get 2-chloro-8-methyl-9H-purin-6-amine (700 mg, 94% yield) as a yellow solid.

### Step 4

To a solution of 2-chloro-8-methyl-9H-purin-6-amine (700 mg, 3.81 mmol, 1.0 eq) in DMSO (20 mL), Cu(OAc)2 (910 mg, 4.58 mmol, 1.2 eq), pyridine (900 mg, 11.4 mmol, 3.0 eq) and pyridine-4-boronic acid (700 mg, 5.72 mmol, 1.5 eq) were added. The mixture was heated at 40° C. for 15 hours. The reaction was quenched with EDTA solution (20 mL) and then it was filtered, the filtrate was concentrated to give 2-chloro-8-methyl-9-(pyridin-4-yl)-9H-purin-6-amine (700 mg, 70% yield) as a yellow solid.

### Step 5

A mixture of 2-chloro-8-methyl-9-(pyridin-4-yl)-9H-purin-6-amine (100 mg, 0.38 mmol, 1.0 eq) and 4-(piperazin-1-yl)phenol (171 mg, 0.96 mmol, 2.5 eq) in NMP (3 mL) was heated in microwave at 130° C. for 1 hour. After cooling, the reaction mixture was quenched with water (10 mL) and then it was filtered. The filtrate was concentrated and the residue was purified by preparative HPLC to give 1-(9-(2-hydroxyethyl)-6-(1H-indazol-1-yl)-9H-purin-2-yl)-4-(4-hydroxyphenyl)piperidin-4-ol (2.4 mg, 1.6% yield) as a white solid.

¹H NMR (400 MHz, DMSO-d6): δ 8.84 (s, 1H), 8.79-8.77 (m, 2H), 7.67-7.65 (m, 2H), 6.91 (br, 2H), 6.81 (d, J=9.2 Hz, 2H), 6.65 (d, J=9.2 Hz, 2H), 3.76-3.68 (m, 4H), 2.95-2.89 (m, 4H), 2.44 (s, 3H); LC/MS(ESI-MS). [M+H]+=403.2.

### Example 219. 4-(4-hydroxyphenyl)-1-(6-(pyridin-4-ylamino)-9H-purin-2-yl)piperidin-4-ol

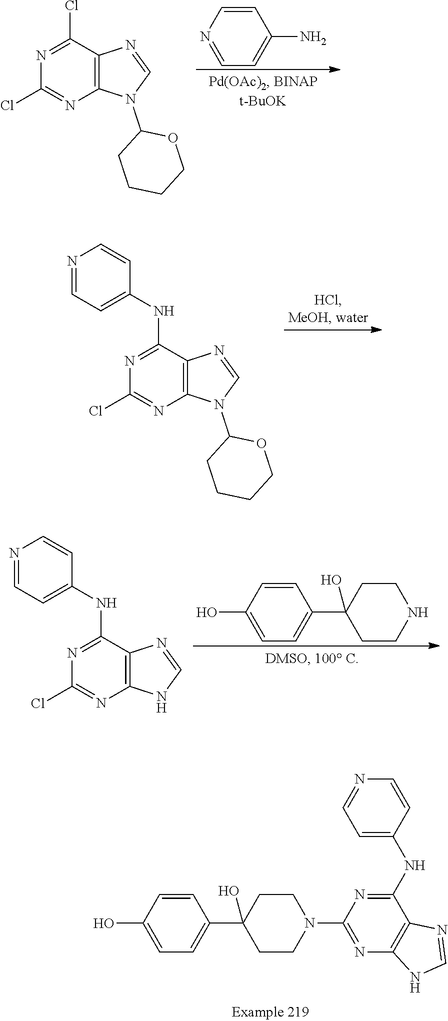 [SMILES: Clc1nc(Cl)c2ncn(C3CCCCO3)c2n1 ; Nc1ccncc1 ; Clc1nc(Nc2ccncc2)c2ncn(C3CCCCO3)c2n1 ; Clc1nc(Nc2ccncc2)c2nc[nH]c2n1 ; Oc1ccc(C2(O)CCNCC2)cc1 ; Oc1ccc(C2(O)CCN(c3nc(Nc4ccncc4)c4nc[nH]c4n3)CC2)cc1]

### Step 1

To a solution of 2,6-dichloro-9-(tetrahydro-2H-pyran-2-yl)-9H-purine (500 mg, 1.831 mmol, 1 eq) in toluene (10 mL) were added Pd(OAc)2 (12.33 mg, 0.055 mmol, 0.05 eq), BINAP (113.9 mg, 0.183 mmol, 0.1 eq), t-BuOK (616.27 mg, 5.492 mmol, 3 eq) and pyridin-4-amine (0.15 mL, 1.648 mmol, 0.9 eq). The reaction was heated at 80° C. for 15 hours. The reaction was cooled to room temperature and added water (15 mL). The mixture was extracted with ethyl acetate (15 mL×3). The combined organic layers were washed with brine, dried with Na₂SO₄, filtered and concentrated. The residue was purified by silica column chromatography to get 2-chloro-N-(pyridin-4-yl)-9-(tetrahydro-2H-pyran-2-yl)-9H-purin-6-amine (200 mg, 33% yield) as a white solid.

### Step 2

To a solution of 2-chloro-N-(pyridin-4-yl)-9-(tetrahydro-2H-pyran-2-yl)-9H-purin-6-amine (200 mg, 0.605 mmol, 1.0 eq) in MeOH (5 mL), was added 2N HCl (2 mL), the mixture was stirred at room temperature for 2 hours. Water (5 mL) was added and the mixture was extracted with ethyl acetate (15 mL×3). The combined organic layers were washed with brine, dried with Na₂SO₄, filtered and concentrated to get 2-chloro-N-(pyridin-4-yl)-9H-purin-6-amine (100 mg, 67% yield) which was used in the next step without further purification.

### Step 3

To a solution of 2-chloro-N-(pyridin-4-yl)-9H-purin-6-amine (100 mg, 0.405 mmol, 1.0 eq) in DMSO (3 mL), 4-(4-hydroxyphenyl)piperidin-4-ol (196 mg, 1.14 mmol, 2.5 eq) and DIEA (157 mg, 1.2 mmol, 3 eq) was added. The mixture was heated to 100° C. for 2 hours. The mixture diluted with water (10 mL) to obtain solid by filtration as the crude product. It was further purified by silica gel column chromatography (DCM:MeOH=10:1, v/v) to give 4-(4-hydroxyphenyl)-1-(6-(pyridin-4-ylamino)-9H-purin-2-yl)piperidin-4-ol (8 mg, 4.9% yield) as a white solid.

¹H NMR (400 MHz, DMSO-d6): δ 12.48 (s, 1H), 9.71 (s, 1H), 9.18-9.11 (m, 2H), 8.26-8.24 (m, 1H), 8.18-8.17 (m, 2H), 7.90 (s, 1H), 7.35-7.31 (m, 1H), 7.26 (d, J=8.4 Hz, 2H), 6.67 (d, J=8.8 Hz, 2H), 4.88 (s, 1H), 4.46-4.43 (m, 2H), 3.34-3.27 (m, 2H), 1.89-1.83 (m, 2H), 1.64 (d, J=9.2 Hz, 2H); LC/MS(ESI-MS): [M+H]+=404.1.

### Example 220. 4-(4-hydroxyphenyl)-1-(6-(methyl(pyridin-4-yl)amino)-9H-purin-2-yl)piperidin-4-ol

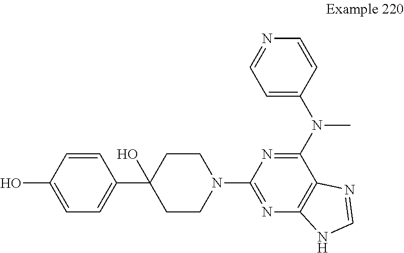 [SMILES: CN(c1ccncc1)c1nc(N2CCC(O)(c3ccc(O)cc3)CC2)nc2[nH]cnc12]

The proceeding example was prepared according to Example 219 by substituting pyridin-4-amine with N-methylpyridin-4-amine. The desired product 4-(4-hydroxy phenyl)-1-(6-(methyl(pyridin-4-yl)amino)-9H-purin-2-yl)piperidin-4-ol was isolated as a white solid.

¹H NMR (400 MHz, DMSO-d6): δ9.06 (br, 1H), 8.52 (d, J=7.2 Hz, 2H), 8.17 (s, 1H), 7.59 (d, J=7.2 Hz, 2H), 6.95 (br, 2H), 6.71 (d, J=8.4 Hz, 2H), 3.91-3.75 (m, 4H), 3.84 (s, 3H), 3.19-3.11 (m, 4H); LC/MS(ESI-MS): [M+H]+=403.2.

### Example 221. 2-(2-(3-hydroxy-3-phenylazetidin-1-yl)-6-(1H-indazol-1-yl)-9H-purin-9-yl)acetic acid

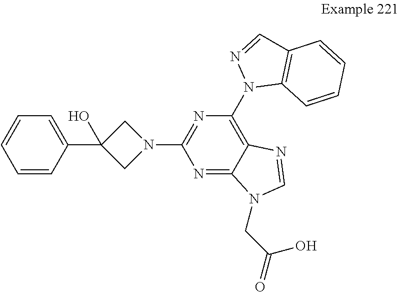 [SMILES: O=C(O)Cn1cnc2c(-n3ncc4ccccc43)nc(N3CC(O)(c4ccccc4)C3)nc21]

The proceeding example was prepared according to Example 21 by substituting 6-(piperazin-1-yl)pyridin-3-ol with 3-phenylazetidin-3-ol. The desired product 2-(2-(3-hydroxy-3-phenylazetidin-1-yl)-6-(1H-indazol-1-yl)-9H-purin-9-yl)acetic acid was isolated as a white solid.

¹H NMR (400 MHz, DMSO-d6): δ 8.77-8.61 (m, 4H), 7.99-7.95 (m, 1H), 7.72-7.63 (m, 3H), 7.48-7.40 (m, 3H), 7.33-7.29 (m, 1H), 5.15 (s, 1H), 5.04 (s, 2H), 4.52-4.33 (m, 4H); LC/MS(ESI-MS): [M+H]+=442.1.

### Example 222. 1-(6-(1H-indazol-1-yl)-9-(prop-2-yn-1-yl)-9H-purin-2-yl)-4-(4-hydroxyphenyl)piperidin-4-ol

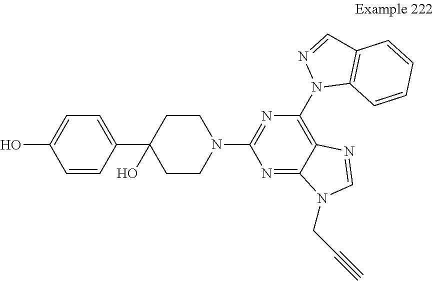 [SMILES: C#CCn1cnc2c(-n3ncc4ccccc43)nc(N3CCC(O)(c4ccc(O)cc4)CC3)nc21]

The proceeding example was prepared according to Example 21 by substituting 6-(piperazin-1-yl)pyridin-3-ol with 4-(4-hydroxyphenyl)piperidin-4-ol, ethyl 2-bromoacetate with bromoprop-1-yne. The desired product 1-(6-41H-indazol-1-yl)-9-(prop-2-yn-1-yl)-9H-purin-2-yl)-4-(4-hydroxyphenyl)piperidin-4-ol was isolated as a red solid.

¹H NMR (400 MHz, DMSO-d6): δ; LC/MS(ESI-MS): [M−H]−=466.2.

### Example 223. 2-(2-(4-(4-fluorophenyl)-4-hydroxypiperidin-1-yl)-6-(1H-indazol-1-yl)-9H-purin-9-yl)acetic acid

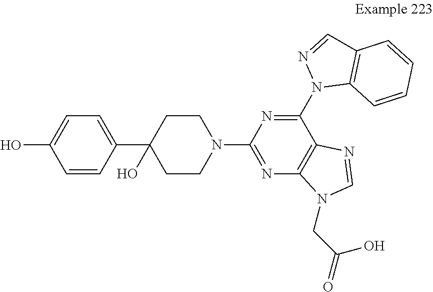 [SMILES: O=C(O)Cn1cnc2c(-n3ncc4ccccc43)nc(N3CCC(O)(c4ccc(O)cc4)CC3)nc21]

The proceeding example was prepared according to Example 21 by substituting 6-(piperazin-1-yl)pyridin-3-ol with 4-(4-fluorophenyl)piperidin-4-ol. The desired product 2-(2-(4-(4-fluorophenyl)-4-hydroxypiperidin-1-yl)-6-(1H-indazol-1-yl)-9H-purin-9-yl)acetic acid was isolated as a yellow solid.

¹H NMR (400 MHz, DMSO-d6): δ 8.60-8.56 (m, 2H), 8.07 (s, 1H), 7.94 (d, J=8.0 Hz, 1H), 7.62-7.53 (m, 3H), 7.37 (t, J=7.2 Hz, 1H), 7.11 (t, J=8.4 Hz, 2H), 5.25 (s, 1H), 4.71-4.58 (m, 4H), 2.02-1.97 (m, 2H), 1.77-1.74 (m, 2H); LC/MS(ESI-MS): [M−H]−=488.1.

### Example 224. 4-(4-fluorophenyl)-1-(9-(2-hydroxyethyl)-6-(1H-indazol-1-yl)-9H-purin-2-yl)piperidin-4-ol

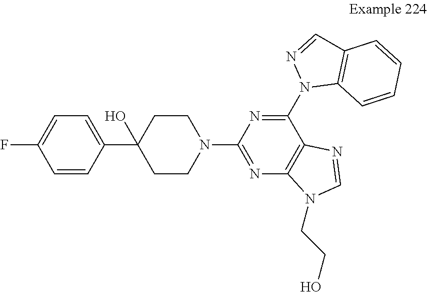 [SMILES: OCCn1cnc2c(-n3ncc4ccccc43)nc(N3CCC(O)(c4ccc(F)cc4)CC3)nc21]

The proceeding example was prepared according to Example 3 by substituting 4-(piperazin-1-yl)phenol with 4-(4-fluorophenyl)piperidin-4-ol. The desired product 4-(4-fluorophenyl)-1-(9-(2-hydroxyethyl)-6-(1H-indazol-1-yl)-9H-purin-2-yl)piperidin-4-ol was isolated as a white solid.

¹H NMR (400 MHz, DMSO-d6): δ8.59-8.56 (m, 2H), 8.11 (s, 1H), 7.94 (d, J=8.0 Hz, 1H), 7.63-7.35 (m, 3H), 7.39-7.35 (m, 1H), 7.14-7.09 (m, 2H), 5.24 (s, 1H), 5.01 (s, 1H), 4.73-4.60 (m, 2H), 4.21 (t, J=5.6 Hz, 2H), 3.84-3.78 (m, 2H), 3.51-3.41 (m, 2H), 2.09-1.95 (m, 2H), 1.79-1.72 (m, 2H); LC/MS(ESI-MS): [M+H]+=474.2.

### Example 225. 1-(9-(2-hydroxyethyl)-6-(1H-indazol-1-yl)-9H-purin-2-yl)-3-phenylazetidin-3-ol

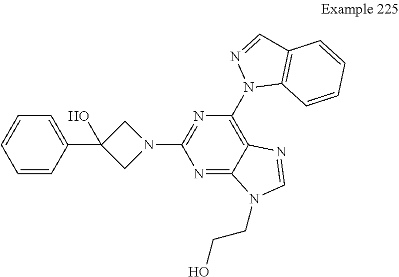 [SMILES: OCCn1cnc2c(-n3ncc4ccccc43)nc(N3CC(O)(c4ccccc4)C3)nc21]

The proceeding example was prepared according to Example 3 by substituting 4-(piperazin-1-yl)phenol with 3-phenylazetidin-3-ol. The desired product 1-(9-(2-hydroxy ethyl)-6-(1H-indazol-1-yl)-9H-purin-2-yl)-3-phenylazetidin-3-ol was isolated as a white solid.

¹H NMR (400 MHz, DMSO-d6): δ 8.76 (d, J=8.4 Hz, 2H), 8.69 (s, 1H), 7.97 (d, J=7.6 Hz, 1H), 7.65-7.63 (m, 3H), 7.44-7.40 (m, 3H), 7.36-7.28 (m, 1H), 4.52-4.34 (m, 4H), 4.30-4.25 (m, 2H), 3.95-3.83 (m, 2H); LC/MS(ESI-MS): [M+H]+=428.2.

### Example 226. 1-(9-(2-hydroxyethyl)-6-(1H-indazol-1-yl)-9H-purin-2-yl)-3-phenylazetidin-3-ol

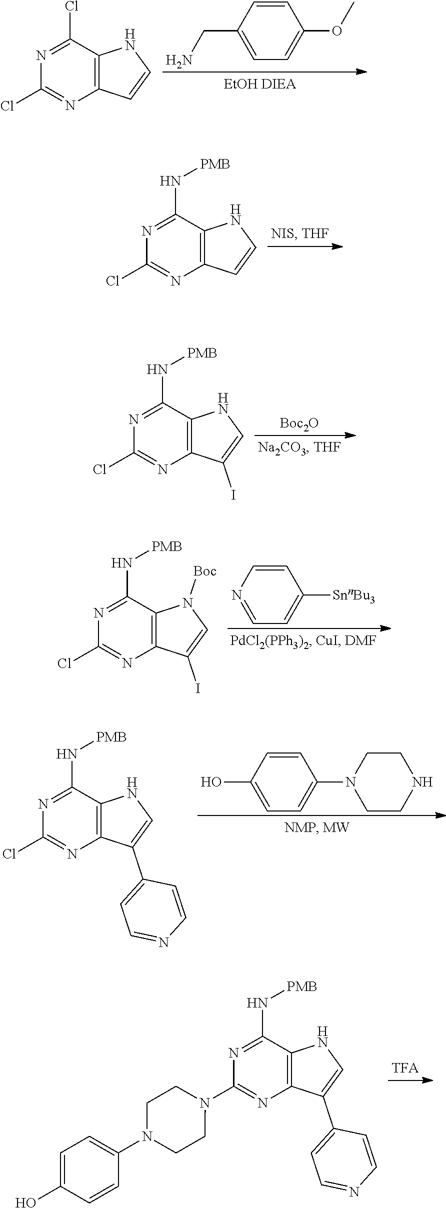

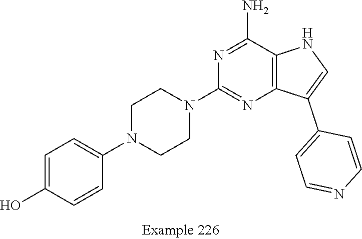 [SMILES: Nc1nc(N2CCN(c3ccc(O)cc3)CC2)nc2c(-c3ccncc3)c[nH]c12]

### Step 1

To a solution of 2,4-dichloro-5H-pyrrolo[3,2-d]pyrimidine (2.4 g, 12.7 mmol, 1.0 eq) in EtOH (15 mL), (4-methoxyphenyl)methanamine (2.45 g, 17.9 mmol, 1.4 eq) and DIEA (2.47 g, 19.1 mmol, 1.5 eq) were added. The mixture was heated at 70° C. The reaction mixture was concentrated under vacuum and slurried with PE to give, the solid was collected to get product 2-chloro-N-(4-methoxybenzyl)-5H-pyrrolo[3,2-d]pyrimidin-4-amine (1.2 g, 33% yield) as a yellow oil.

### Step 2

To a solution of 2-chloro-N-(4-methoxybenzyl)-5H-pyrrolo[3,2-d]pyrimidin-4-amine (800 mg, 2.78 mmol, 1 eq) in THF (10 mL), NIS (624 mg, 2.78 mmol, 1 eq) was added. The mixture was stirred at room temperature for 1 hour. The solvent was concentrated under vacuum to get 2-chloro-7-iodo-N-(4-methoxybenzyl)-5H-pyrrolo[3,2-d]pyrimidin-4-amine (1.1 g, 100% yield) as a crude product to use in the next step without purification.

### Step 3

To a solution of 2-chloro-7-iodo-N-(4-methoxybenzyl)-5H-pyrrolo[3,2-d]pyrimidin-4-amine (1.1 g, 2.65 mmol, 1 eq) in THF (10 mL), di-tert-butyl dicarbonate (2.9 g, 13.3 mmol, 5 eq) and Na2CO3 (1.4 g, 13.3 mmol, 5 eq) were added. The reaction mixture was stirred at room temperature for 1 hour. The solvent of the mixture was concentrated under vacuum. The residue was extracted with ethyl acetate (2×20 mL). The combined organic layers were dried with sodium sulfate, filtered and concentrated under vacuum. The residue was purified by silica-gel chromatography (EA:PE=1:10, v/v) to get tert-butyl 2-chloro-7-iodo-4-((4-methoxybenzyl)amino)-5H-pyrrolo[3,2-d]pyrimidine-5-carboxylate (520 mg, 38% yield) as a white solid.

### Step 4

To a solution of tert-butyl 2-chloro-7-iodo-4-((4-methoxybenzyl)amino)-5H-pyrrolo[3,2-d]pyrimidine-5-carboxylate (250 mg, 0.49 mmol, 1 eq) in DMF (5 mL), was added 4-pyridyl-tri(nbutyl)tin (894 mg, 2.4 mmol, 5 eq), PdCl2 (PPh3)2 (68 mg, 0.1 mmol, 0.2 eq) and CuI (9.2 mg, 0.05 mmol, 0.1 eq). The mixture was heated at 100° C. under N₂. The reaction mixture was diluted with ethyl acetate (20 mL) and the pH of the mixture was adjusted to 3-4 with aqueous 1N HCl. The reaction mixture was extracted with ethyl acetate (3×10 mL). The combined organic layers were washed with brine, dried over sodium sulfate, filtered and concentrated under vacuum to give 2-chloro-N-(4-methoxybenzyl)-7-(pyridin-4-yl)-5H-pyrrolo[3,2-d]pyrimidin-4-amine (160 mg, 94% yield) as a yellow solid.

### Step 5

To a solution of 2-chloro-N-(4-methoxybenzyl)-7-(pyridin-4-yl)-5H-pyrrolo[3,2-d]pyrimidin-4-amine (160 mg, 0.44 mmol, 1 eq) in NMP (2 mL), 4-(piperazin-1-yl)phenol (194.9 mg, 1.1 mmol, 2.5 eq) was added and the mixture was heated with microware at 130° C. The reaction was cooled and water (2 mL) was added to get a precipitate. The solid was collected to afford 4-(4-(4-((4-methoxybenzyl)amino)-7-(pyridin-4-yl)-5H-pyrrolo[3,2-d]pyrimidin-2-yl)piperazin-1-yl)phenol (130 mg, 58% yield) was a yellow solid.

### Step 6

To a solution of 4-(4-(4-((4-methoxybenzyl)amino)-7-(pyridin-4-yl)-5H-pyrrolo[3,2-d]pyrimidin-2-yl)piperazin-1-yl)phenol (130 mg, 0.26 mmol, 1 eq) in TFA (3 mL), the mixture was heated at 40° C. for 1 hour. The reaction mixture was cooled and the solvent was evaporated under vacuum. Water (5 mL) was added in the mixture and washed with ethyl acetate. Adjusted the pH of aqueous layer to 8-9 using sodium bicarbonate to obtain a solid as the crude product. Purified the crude by preparative HPLC to get 4-(4-(4-amino-7-(pyridin-4-yl)-5H-pyrrolo[3,2-d]pyrimidin-2-yl)piperazin-1-yl)phenol (5 mg, 5% yield) as a white solid.

¹H NMR (400 MHz, DMSO-d6): δ11.17 (s, 1H), 8.83 (s, 1H), 8.47-8.46 (m, 2H), 8.14-8.07 (m, 3H), 6.86 (d, J=8.8 Hz, 2H), 6.78-6.63 (m, 4H), 3.90-3.75 (m, 4H), 3.11-2.97 (m, 4H); LC/MS(ESI-MS): [M+H]+=388.2.

### Example 227. 2-(4-phenyl-1,4-diazepan-1-yl)-9-(pyridin-4-yl)-9H-purin-6-amine

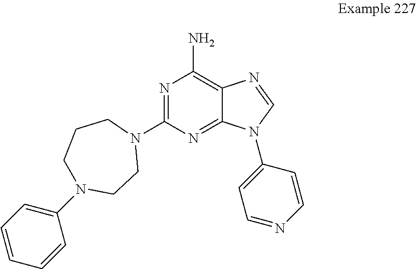 [SMILES: Nc1nc(N2CCCN(c3ccccc3)CC2)nc2c1ncn2-c1ccncc1]

The proceeding example was prepared according to Example 66 by substituting 4-(piperazin-1-yl)phenol with 1-phenyl-1,4-diazepane, phenyl boronic acid with pyridin-4-ylboronic acid. The desired 2-(4-phenyl-1,4-diazepan-1-yl)-9-(pyridin-4-yl)-9H-purin-6-amine was isolated as a white solid.

¹H NMR (400 MHz, DMSO-d6): δ 8.85-8.73 (m, 2H), 8.55 (s, 1H), 8.41-8.26 (m, 2H), 7.14-7.01 (m, 4H), 6.76 (d, J=8.0 Hz, 2H), 6.55 (t, J=7.2 Hz, 1H), 3.95-3.91 (m, 2H), 3.63-3.47 (m, 3H), 3.45-3.41 (m, 3H), 2.09-1.98 (m, 2H); LC/MS(ESI-MS): [M+H]+=387.2.

### Example 228. 6-(4-(6-(1H-indazol-1-yl)-9-(tetrahydrofuran-3-yl)-9H-purin-2-yl)piperazin-1-yl)pyridin-3-ol

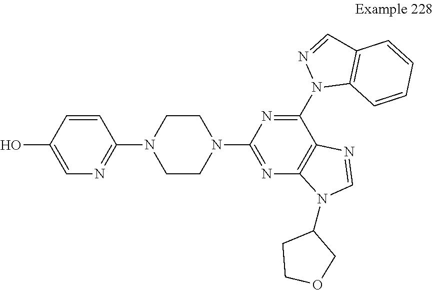 [SMILES: Oc1ccc(N2CCN(c3nc(-n4ncc5ccccc54)c4ncn(C5CCOC5)c4n3)CC2)nc1]

The proceeding example was prepared according to Example 187 by substituting 3,4-dihydro-2H-pyran with tetrahydrofuran-3-yl methanesulfonate, 4-(piperazin-1-yl)phenol with 1-(5-(benzyloxy)pyridin-2-yl)piperazine. The desired 6-(4-(6-(1H-indazol-1-yl)-9-(tetrahydrofuran-3-yl)-9H-purin-2-yl)piperazin-1-yl)pyridin-3-ol was isolated as a white solid.

¹H NMR (400 MHz, DMSO-d6): δ 9.60 (br, 1H), 8.73-8.63 (m, 3H), 7.99 (d, J=8.0 Hz, 1H), 7.71-7.67 (m, 2H), 7.48-7.40 (m, 2H), 7.11-6.98 (m, 1H), 5.30 (s, 1H), 4.18-3.87 (m, 8H), 3.64-3.58 (m, 4H), 2.51-2.39 (m, 2H); LCMS(ESI-MS): [M+H]+=484.2.

### Example 229. 4-(4-(6-methyl-9-(pyridin-3-yl)-9H-purin-2-yl)piperazin-1-yl)phenol

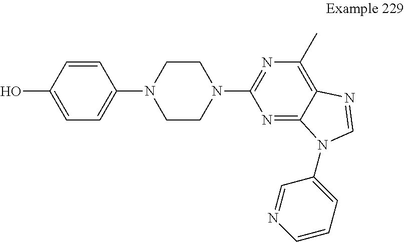 [SMILES: Cc1nc(N2CCN(c3ccc(O)cc3)CC2)nc2c1ncn2-c1cccnc1]

The proceeding example was prepared according to Example 43 by substituting phenylboronic acid with pyridin-3-ylboronic acid. The desired 44-(4-(6-methyl-9-(pyridin-3-yl)-9H-purin-2-yl)piperazin-1-yl)phenol was isolated as a gray solid.

¹H NMR (400 MHz, DMSO-d6): δ8.87 (s, 1H), 8.80-8.75 (m, 3H), 8.19-8.17 (m, 2H), 6.86 (d, J=9.2 Hz, 2H), 6.68 (d, J=8.8 Hz, 2H), 3.95-3.92 (m, 4H), 3.08-3.05 (m, 4H), 2.61 (s, 3H); LCMS(ESI-MS): [M+H]+=388.1.

### Example 230. 6-(4-(4-amino-7-(pyridin-4-yl)-7H-pyrrolo[2,3-d]pyrimidin-2-yl)piperazin-1-yl)pyridin-3-ol

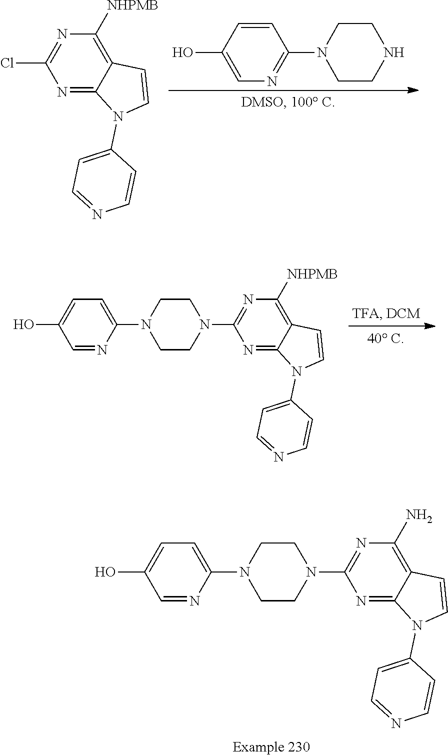

### Step 1

To a solution of 2-chloro-N-(4-methoxybenzyl)-7-(pyridin-4-yl)-7H-pyrrolo[2,3-d]pyrimidin-4-amine (50 mg, 0.14 mmol, 1.0 eq) in DMSO (3 mL), 6-(piperazin-1-yl)pyridin-3-ol (62 mg, 0.34 mmol, 2.5 eq) was added. The mixture heated to 100° C. for 15 hours. The mixture was cooled to room temperature. The mixture was diluted with water (10 mL) to collect the solid by filtration to give 6-(4-(4-((4-methoxybenzyl)amino)-7-(pyridin-4-yl)-7H-pyrrolo[2,3-d]pyrimidin-2-yl)piperazin-1-yl)pyridin-3-ol (80 mg, 100% yield) as a black solid. The product was used in the next step without further purification.

### Step 2

To a solution of 6-(4-(4-((4-methoxybenzyl)amino)-7-(pyridin-4-yl)-7H-pyrrolo[2,3-d]pyrimidin-2-yl)piperazin-1-yl)pyridin-3-ol (80 mg, 0.16 mmol, 1 eq) in DCM (2 mL), TFA (2 mL) was added and the mixture was heated to 40° C. for 15 hours. The reaction mixture was concentrated under reduced pressure. The residue was purified by preparative HPLC to give 6-(4-(4-amino-7-(pyridin-4-yl)-7H-pyrrolo[2,3-d]pyrimidin-2-yl)piperazin-1-yl)pyridin-3-ol (2 mg, 3% yield) as a yellow solid.

¹H NMR (400 MHz, DMSO-d6): δ 8.87 (d, J=6.4 Hz, 2H), 8.72 (d, J=6.0 Hz, 2H), 7.71-7.68 (m, 2H), 7.34-7.22 (m, 2H), 7.03-6.98 (m, 1H), 6.85 (d, J=4.0 Hz, 1H), 3.91-3.83 (m, 4H), 3.64-3.57 (m, 4H); LC/MS(ESI-MS): [M+H]+=487.2.

### Biological Assay and Data

As stated above, the compounds of Formula I are MIF inhibitors, and are useful in the treatment of diseases mediated by MIF. The biological activities of the compounds of Formula I can be determined by using any suitable assay for determining the activity of a candidate compound as a MIF inhibitor, as well as tissue and in vivo models.

### Example 1: MIF Enzymatic Assays

Tautomerase Assay Using pHPP as Substrate

This assay measured MIF's tautomerase activity in a cell-free system and was based on the determination of initial rates of the MIF-catalyzed conversion of the ketonic into the enolic tautomer of pHPP. This was achieved by spectrophotometric quantification of the complex between borate and the product of the reaction (enolic pHPP). The substrate was prepared by conversion of the enolic pHPP into its ketonic form. To achieve this, 0.5 M pHPP in methanol was diluted 10-fold with 50 mM sodium acetate buffer at pH 6.0, and then the suspension was shaken for 24 h at room temperature in darkness, and finally stored at 4° C. for not more than 1 week, with 5 min sonication being recommended before use.

Assays were performed in small-volume clear-bottom black 96 or 384-well polystyrene plates (Greiner Bio-One). First, 2 μL of the enzyme solution containing 6 nM of MIF in DPBS, 0.025% w/v BSA, and 300 μM CHAPS was dispensed onto sample and negative control wells using Multidrop Combi with metallic tip cassettes (Thermo Fisher Scientific) previously treated with Sigmacote. Then, 2 μL of the same buffer without MIF was dispensed onto positive control wells. The reaction was started by addition of the following to all wells: 2 μL of substrate solution containing 3 mM ketonic pHPP in 200 mM boric acid, 25 mM sodium phosphate, 0.025% w/v BSA, and 300 μM CHAPS at pH 6.0. In order to remove bubbles, the plate was centrifuged in an Allegra 25R centrifuge (Beckman Coulter, Inc., Brea, Calif.) at 1000 rpm for 2 min at room temperature. Then, the plate was read in an EnVision. Final concentrations of enzyme and substrate were 3 nM and 1.5 mM, respectively. Initial rates were calculated for each well as the slope of the absorbance progress curve.

All exemplified compounds (Examples 1-230) were tested in the MIF tautomerase assay or a similar assay described above. The data mentioned below represents a mean pIC₅₀ value of multiple test results. It is understood that the data illustrated below may have reasonable variation depending on the specific conditions and procedures used by the person conducting the testing.

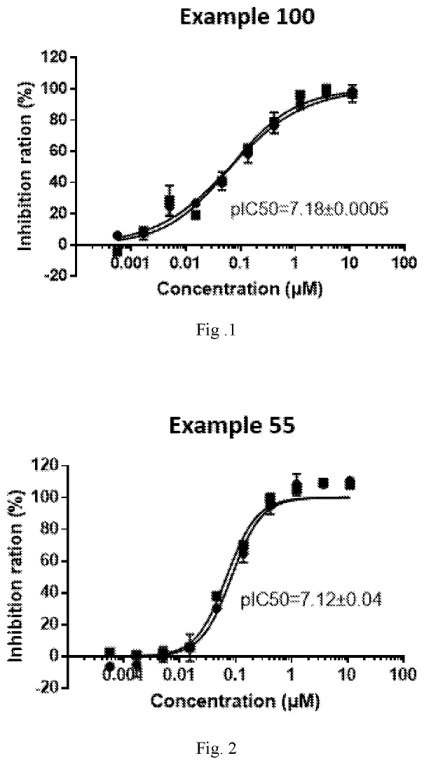

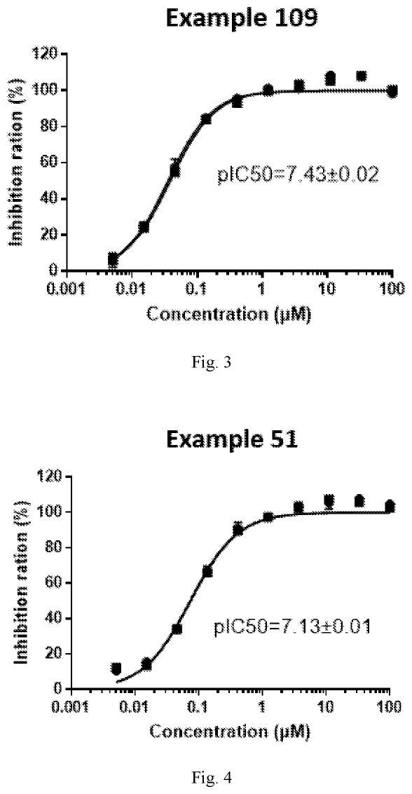

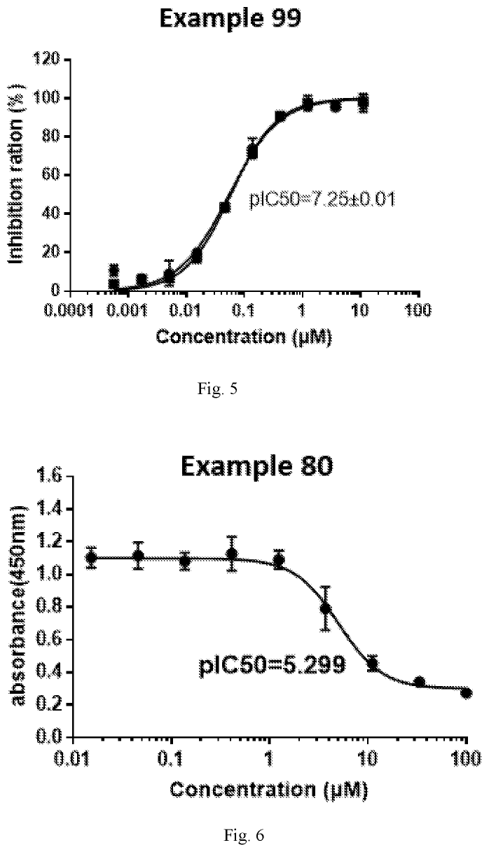

All exemplified compound exhibited a pIC₅₀≥5.0. As shown in FIGS. 1-5, the compounds of Examples 51, 55, 99, 100, and 109 exhibited a pIC₅₀≥7.0.

### Assay Data

The pIC₅₀ data from assays for measuring the inhibitory effect on MIF by compounds of Formula (I) are listed in table I below. Tautomerase inhibition pIC₅₀: A means <5, B means 5.01 to 6, C means 6.01 to 7, D means 7.01 to 8).

TABLE 1
Inhibition of the MIF Enzyme in vitro by Compounds of Fomrula (I)

| Example | Tautomerase |
|---|---|
| # | Inhibition pIC₅₀ |
| 1 | B |
| 2 | C |
| 3 | C |
| 4 | A |
| 5 | A |
| 6 | A |
| 7 | B |
| 8 | C |
| 9 | C |
| 10 | D |
| 11 | D |
| 12 | C |
| 13 | A |
| 14 | B |
| 15 | B |
| 16 | D |
| 17 | C |
| 18 | D |
| 19 | B |
| 20 | D |
| 21 | D |
| 22 | B |
| 23 | C |
| 24 | D |
| 25 | B |
| 26 | C |
| 27 | A |
| 28 | B |
| 29 | A |
| 30 | B |
| 31 | C |
| 32 | B |
| 33 | D |
| 34 | B |
| 35 | C |
| 36 | C |
| 37 | B |
| 38 | B |
| 39 | C |
| 40 | B |
| 41 | D |
| 42 | B |
| 43 | C |
| 44 | C |
| 45 | D |
| 46 | C |
| 47 | C |
| 48 | C |
| 49 | A |
| 50 | B |
| 51 | D |
| 52 | D |
| 53 | D |
| 54 | C |
| 55 | D |
| 56 | C |
| 57 | D |
| 58 | B |
| 59 | C |
| 60 | C |
| 61 | B |
| 62 | D |
| 63 | A |
| 64 | B |
| 65 | B |
| 66 | B |
| 67 | B |
| 68 | A |
| 69 | A |
| 70 | A |
| 71 | B |
| 72 | B |
| 73 | A |
| 74 | C |
| 75 | A |
| 76 | A |
| 77 | B |
| 78 | B |
| 79 | A |
| 80 | D |
| 81 | B |
| 82 | C |
| 83 | B |
| 84 | B |
| 85 | C |
| 86 | C |
| 87 | B |
| 88 | B |
| 89 | B |
| 90 | B |
| 91 | C |
| 92 | B |
| 93 | C |
| 94 | C |
| 95 | B |
| 96 | B |
| 97 | C |
| 98 | C |
| 99 | D |
| 100 | D |
| 101 | C |
| 102 | B |
| 103 | B |
| 104 | B |
| 105 | C |
| 106 | C |
| 107 | D |
| 108 | D |
| 109 | D |
| 110 | A |
| 111 | B |
| 112 | C |
| 113 | C |
| 114 | C |
| 115 | C |
| 116 | C |
| 117 | C |
| 118 | B |
| 119 | C |
| 120 | D |
| 121 | D |
| 122 | D |
| 123 | D |
| 124 | D |
| 125 | C |
| 126 | C |
| 127 | D |
| 128 | D |
| 129 | A |
| 130 | A |
| 131 | C |
| 132 | D |
| 133 | C |
| 134 | D |
| 135 | D |
| 136 | B |
| 137 | B |
| 138 | C |
| 139 | B |
| 140 | A |
| 141 | D |
| 142 | D |
| 143 | C |
| 144 | C |
| 145 | D |
| 146 | D |
| 147 | B |
| 148 | D |
| 149 | D |
| 150 | D |
| 151 | D |
| 152 | D |
| 153 | B |
| 154 | A |
| 155 | D |
| 156 | A |
| 157 | D |
| 158 | C |
| 159 | A |
| 160 | A |
| 161 | A |
| 162 | C |
| 163 | D |
| 164 | D |
| 165 | D |
| 166 | D |
| 167 | D |
| 168 | D |
| 169 | D |
| 170 | D |
| 171 | D |
| 172 | D |
| 173 | D |
| 174 | D |
| 175 | D |
| 176 | C |
| 177 | C |
| 178 | C |
| 179 | B |
| 180 | A |
| 181 | A |
| 182 | A |
| 183 | A |
| 184 | D |
| 185 | A |
| 186 | C |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | D |
| 205 | C |
| 206 | C |
| 207 | C |
| 208 | C |
| 209 | B |
| 210 | B |
| 211 | B |
| 212 | B |
| 213 | A |
| 214 | A |
| 215 | A |
| 216 | D |
| 217 | D |
| 218 | C |
| 219 | C |
| 220 | A |
| 221 | C |
| 222 | C |
| 223 | C |
| 224 | B |
| 225 | B |
| 226 | A |
| 227 | A |
| 228 | C |
| 229 | D |
| 230 | B |

### Example 2: Cell Proliferation Assay

The assay measures the ability of a compound to inhibit cell proliferation using Cell Counting Kit-8 (CCK-8), which allows sensitive colorimetric assays for the determination of cell viability in cell proliferation and cytotoxicity assays. The highly water-soluble tetrazolium salt, WST-8, is reduced by dehydrogenase activities in cells to give a yellow-color formazan dye, which is soluble in the tissue culture media. The amount of the formazan dye, generated by the activities of dehydrogenases in cells, is directly proportional to the number of living cells.

The assay was performed based on the instruction of the manufacture (Dojindo Molecular Technologies Inc., Rockville, Md., USA). To start the assay, BV2 cells were seeded at a density of 2×106 cells/well into 96-well plates and treated with test compounds, which were used at a top final assay concentration of 33.3 μM, diluted in assay medium with 1:3 dilutions to produce 10 point concentration responses, for 48 h. After incubation, the CCK-8 solution (10 μl) was added to each well of the plate and the plates were incubated in incubator at 37° C. for 3 h. Absorbance was recorded three times independently using a microplate reader (Bio-Rad Laboratories, Richmond, Calif., USA) at 450 nm.

Selected examples were tested in the BV2 cell proliferation assay. The data mentioned below represents a mean pIC₅₀ value of multiple test results. It is understood that the data illustrated below may have reasonable variation depending on the specific conditions and procedures used by the person conducting the testing.

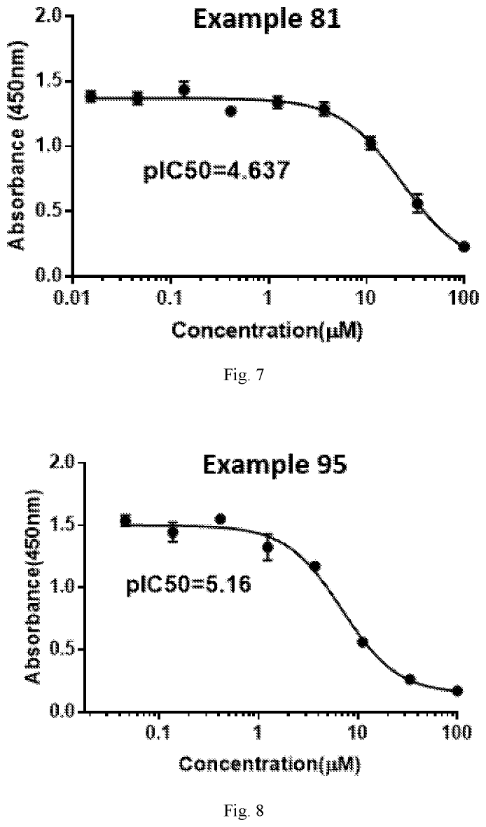

As shown in FIGS. 6-8, the compounds of examples 80, 81 and 95 exhibited a pIC₅₀ of 5.299, 4.637 & 5.16 respectively.

### Example 3: MIF-CD74 Binding Assay

The assay measures the ability of a compound to inhibit the binding of MIF with its receptor CD74 in vitro. 96-well plates were coated with recombinant MIF receptor ectodomain (sCD74=CD7473-232), washed 4 times, and blocked with Superblock (Pierce Biotechnology) at 4° C. overnight. Test compounds, which were used at a top final assay concentration of 33.3 μM, diluted in assay medium with 1:3 dilutions to produce 10 point concentration responses, were pre-incubated with biotinylated human MIF (2 ng/μl) (Roche Applied Sciences) prepared recombinantly for 30 minutes at room temperature. The mixtures then were added to wells for overnight incubation at 4° C. The wells were washed 4 times and strepavidin-conjugated alkaline phosphatase (R&D Systems) was added for one hour incubation at room temperature. After additional washes, 60 μl/well of p-nitrophenyl phosphate (Sigma) substrate was added. Absorbance at 405 nm was plotted as percent A405 relative to wells containing biotinylated human MIF alone. Each plot represented at least three independently performed assays, and each data point depicts a SEM≤10%. Each test compound was analyzed over 10-12 concentrations, and over a ˜500-fold concentration range.

Selected examples were tested in the MIF-CD74 binding assay. The data mentioned below represents a mean pIC50 value of multiple test results. It is understood that the data illustrated below may have reasonable variation depending on the specific conditions and procedures used by the person conducting the testing.

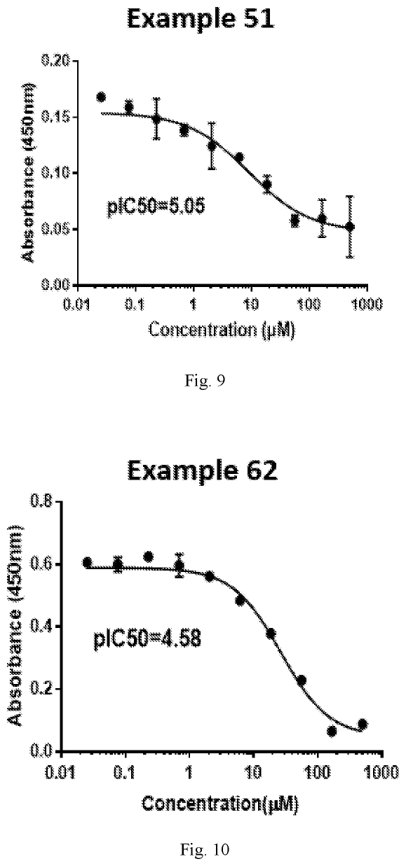

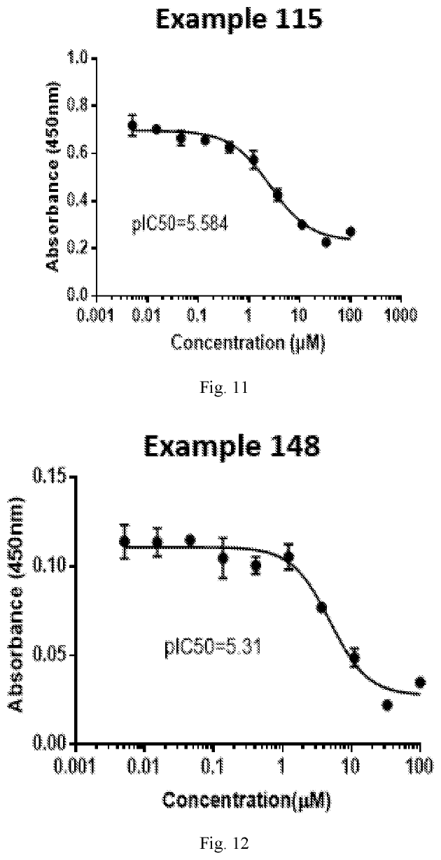

As shown in FIGS. 9-12, the compounds of examples 51, 62, 115 and 148 exhibited a pIC₅₀ of 5.05, 4.58, 5.584 and 5.31 respectively.

### Example 4 Mouse Syngeneic Cancer Models

### Animal Model

Female Balb/c mice (5-6 weeks old) were purchased from Charles River Laboratories (Hollister, Calif.). Animals were housed and treated in accordance with Institutional Animal Care and Use Committee guidelines. For the generation of 4T1 breast cancer model, 1×105 4T1 cells were injected orthotopically into the mammary fat pad. For the generation of lung cancer model, 1×105 LLC cells were injected subcutaneously. For the generation of murine melanoma model, 1×105 B16-F10 cells were injected subcutaneously. Test compounds were administered by I.P. injection in a dose range of 10-100 mg/kg starting on study day 1 (7 days after tumor implantation). Control groups received vehicle (water) twice daily by I.P. injection. Tumor volume measured by digital caliper (length×width×width/2) and body weight were recorded three times per week. Animals were euthanized when tumors necrotized or volumes reached 2000 mm3.

### Statistical Analysis

One-way Anova, student's T test, and Mann-Whitney test performed by GraphPad5 PRISM software (GraphPad Software, Inco, San Diego, Calif., USA) were used according to different experimental conditions. P value<0.05 was considered statistically significant. Significance is indicated in the figures by asterisks *p<0.05, **p<0.01.

MG-321 and Gemcitabine Synergistically Inhibited Breast Cancer Growth

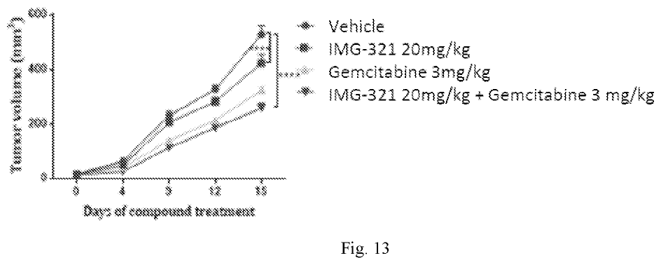

Female Balb/C mice (8 weeks old) were injected with 1×105 4T1 cells into the mammary fat pad. After one week, animals were treated with vehicle or IMG-321 (20 mg/kg, b.i.d.) or gemcitabine (3 mg/kg, once every 10 days) or IMG-321 combined with gemcitabine. Tumor volume was measured by digital caliper (length×width×width/2). Data are mean±SEM. FIG. 13 shows the test compound inhibited the growth of breast cancer.

1. A compound of Formula (I), 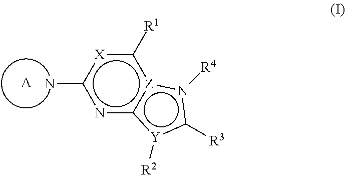
wherein
R¹ is absent, H, alkyl, halo, hydroxyl, alkoxy, thio, thiol ether, monocyclic or bicyclic aryl, monocyclic or bicyclic heteroaryl, monocyclic or bicyclic cycloalkyl, monocyclic or bicyclic heterocycloalkyl, arylalkyl, heteroaryl-alkyl, amino, or hydroxylamino, wherein alkyl, aryl, heteroaryl, cycloalkyl, heterocycloalkyl, arylalkyl, heteroaryl-alkyl, or amino is optionally substituted with one or more substituents each independently selected from the group consisting of halo, cyano, hydroxyl, alkyl, hydroxyalkyl, cycloalkyl, alkoxy, aryl, heteroaryl, alkylcarbonyl, alkoxycarbonyl, alkoxycarbonylalkyl, hydroxycarbonyl, hydroxycarbonylalkyl, hydroxyalkylcarbonyl, hydroxyalkylcarbonylalkyl, alkoxyalkylcarbonylalkyl, aminocarbonylalkyl, amido, hydroxyalkyl, cycloalkyl-alkyl, arylalkyl, and arylalkylhydroxyalkyl, wherein one —CH₂— group in the foregoing heterocycloalkyl is optionally replaced with carbonyl group C═O;
R² is absent, H, halo, cyano, hydroxyl, alkoxy, alkyl, cycloalkyl, heterocycloalkyl, amino, thiol ether, arylalkyl, heteroaryl-alkyl, aryl, or heteroaryl, wherein alkyl, cycloalkyl, heterocycloalkyl, aryl, or heteroaryl is substituted with one or more substituents each independently selected from the group consisting of halo, cyano, alkyl, alkenyl, alkynyl, hydroxyl, alkoxy, hydroxycarbonyl, alkoxycarbonyl, boronic acid, boronic ester, phosphonyl [—CH₂P(O)(OR¹⁰)(OR¹¹], aryl, heteroaryl, aryl-heteroaryl, heteroaryl-aryl, hydroxyaryl-heteroaryl, hydroxyheteroaryl-aryl, amino, aminocarbonyl (amido), alkylsulfonylaminocarbonyl, cycloalkyl-sulfonylaminocarbonyl, alkylsulfonyl, heteroaryl, hydroxycarbonylalkylaminocarbonyl, cycloalkyl, heterocycloalkyl, thiol ether, and aryl;
each of R³ and R⁴, independently, is H or alkyl optionally substituted with one or more substituents each independently selected from the group consisting of halo, cyano, amino, hydroxyl, alkoxy, thiol ether, alkylcarbonyl, hydroxycarbonyl, alkoxycarbonyl, aryl, heteroaryl, cycloalkyl, heterocycloalkyl, and arylalkyl;
X is N or CR⁵, Y is N or C, Z is N or C, provided that when Z is N, X is CR⁵ and Y is C, and when Z is C, X is N and Y is N;
R⁵ is H or alkyl optionally substituted with one or more substituents each independently selected from the group consisting of halo, cyano, amino, hydroxyl, and alkoxy;
Ring A includes the nitrogen atom shown in Figure (I) and is a heteroaryl or heterocyclic group, and Ring A is optionally substituted with one or more substituents each independently selected from the group consisting of aryl, heteroaryl, cycloalkyl, heterocyclic, halo, cyano, alkyl, alkynyl, alkoxycarbonyl, hydroxyl, acyl, aminocarbonyl, alkylsulfonyl, and oxo, wherein aryl, heteroaryl, cycloalkyl, heterocyclic, alkyl, alkynyl, acyl, aminocarbonyl or alkylsulfonyl is optionally substituted with one or more substituents each independently selected from the group consisting of hydroxyl, alkoxy, hydoxyalkyl, amino, aminoalkyl, hydroxycarbonyl, alkoxycarbonyl, hydroxycarbonylalkyl, aminocarbonyl, aminocarbonylalkyl, cycloalkyl, heterocyclic, halo or alkyl;
wherein one —CH₂— group in the foregoing heterocyclic group is optionally replaced with carbonyl group C═O;
or a pharmaceutically acceptable salt, solvent, isomer, or prodrug thereof.

2. The compound of claim 1, wherein R¹ is H, alkyl, monocyclic or bicyclic heteroaryl, monocyclic or bicyclic aryl, fused arylheterocycloalkyl, fused heterocycloalkylaryl, amino, arylamino, heteroarylamino, arylalkylamino, heteroarylalkylamino, heterocycloalkyl, or (cycloalkyl)amino, wherein one —CH2- group in the foregoing heterocycloalkyl is optionally replaced with carbonyl group (C═O), and R¹ is optionally substituted with one or more substituents each independently being halo, alkyl, alkenyl, cycloalkyl, hydroxyl, hydroxyalkyl, alkoxy, amino, cyano, hydroxycarbonyl, hydroxycarbonylalkyl, alkoxycarbonyl, alkoxycarbonylalkyl, aminocarbonyl, alkylaminocarbonyl, hydroxycarbonylalkyl, hydroxycarbonylalkenyl, arylalkylhydroxyalkyl or heteroaryl.

3. The compound of claim 2, wherein the heteroaryl in R¹ is indolinyl, isoindolinyl, indolinonyl, isoindolinonyl, indazolyl, dihydroindenyl, benzotriazolyl, pyridinyltriazolyl, indazolyl, indolyl, pyrrolopyridinyl, or benzoimidazolyl, and the heteroaryl is optionally substituted with one or more substituents each independently being alkyl, hydroxyalkyl, alkoxy, halo, hydroxyl, amino, cyano, hydroxycarbonyl, alkoxycarbonyl, alkylaminocarbonyl, hydroxycarbonlyalkenyl, hydroxycarbonlyalkyl, heteroaryl, or cycloalkyl.

4. The compound of claim 1, wherein R¹ is 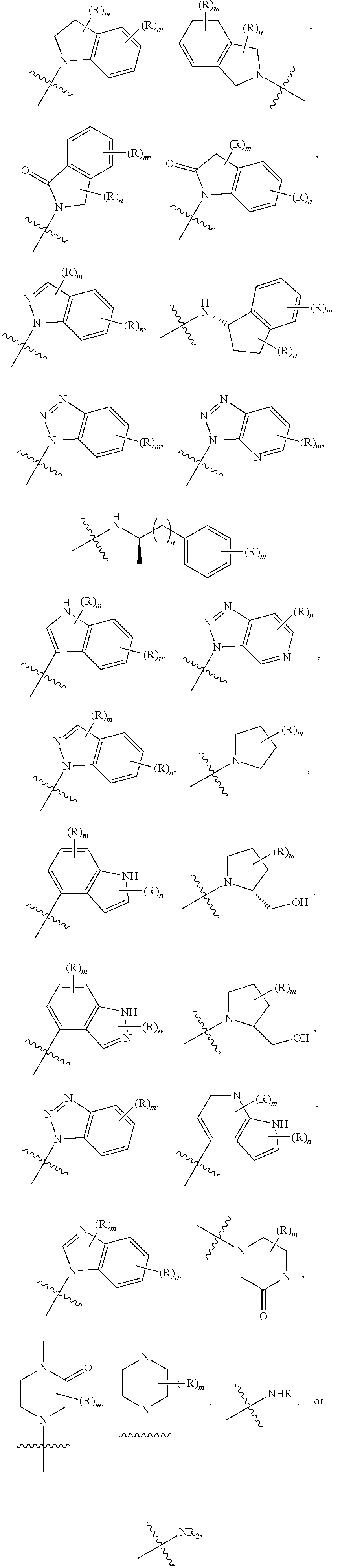
wherein each of m and n is independently 0, 1, or 2; and each R independently is alkyl, cycloalkyl, hydroxyalkyl, alkoxy, halo, amino, cyano, hydroxycarbonyl, alkoxycarbonyl, alkylaminocarbonyl, hydroxycarbonylalkenyl, hydroxycarbonylalkyl, heteroaryl, or aryl.

5. The compound of claim 1, wherein R¹ is H, alkyl, amino, 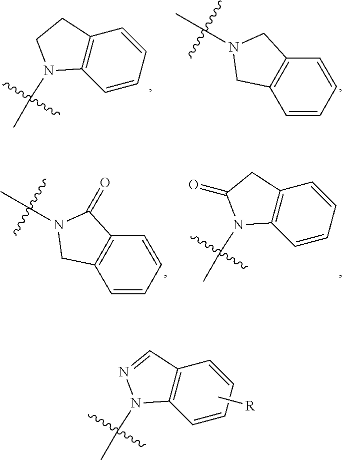
(wherein R is —CH₂OH, —OCH₃, Cl, —OH, —NH₂, —CN, or —COOH), 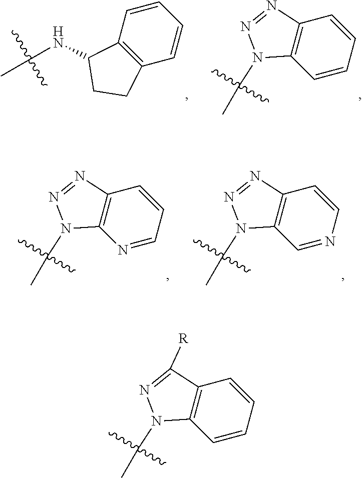
(wherein R is —COOH, —COOEt, —CONHCH₃, —CH₂OH, —CH═CHCOOH, —CH₂CH₂COOH, —CN, -heteroaryl), 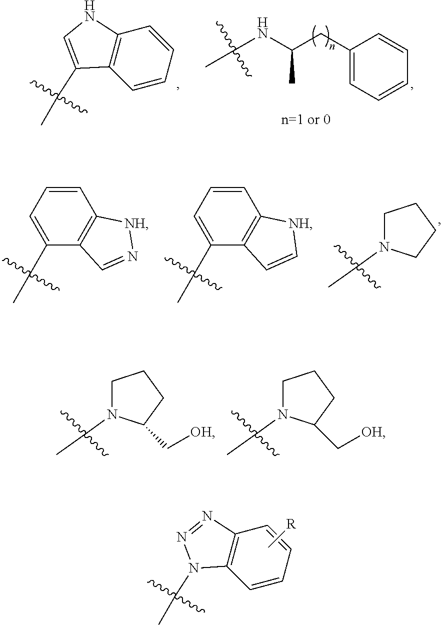
(R is —CH₃, Cl, —COOH, —CH₂OH), 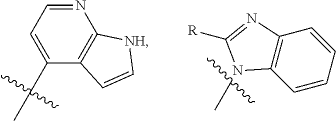
(R is H, —CH₃, —CH₂OH), 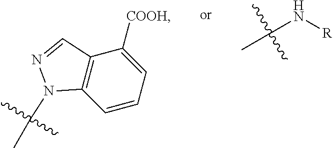
(R is H, hydroxylalkyl, phenylalkyl, cycloalkyl, or cycloalkylalkyl).

6. The compound of claim 1, wherein R² is H, halo, heterocycloalkyl, or alkyl optionally substituted with one or more substituents each independently being amino, aryl, hydroxyl, hydroxycarbonyl, alkoxycarbonyl, aminocarbonyl, arylalkylsulphonylaminocarbonyl, alkylsulphonylaminocarbonyl, cycloalkylsulphonylaminocarbonyl, arylsulphonylaminocarbonyl, alkylcarbonylamino, heteroaryl, or hydroxycarbonylalkylalkylaminocarbonyl; or
R² is aryl or heteroaryl and is optionally substituted with one or more substituents each independently being halo, alkoxy, alkyl, OH, CN, aminocarbonyl, hydroxycarbonyl, alkoxycarbonyl, aryl, heterocycloalkyl, or arylheterocycloalkyl.

7. The compound of claim 6, wherein R² is phenyl or heteroaryl and is optionally substituted with one or more substituents each independently being alkoxy, alkyl, halo, OH, CN, aminocarbonyl, hydroxycarbonyl, alkoxycarbonyl, aryl, heterocycloalkyl, or arylheterocycloalkyl.

8. The compound of claim 6, wherein R² is H, halo, monohydroxyalkyl, dihydroxylalkyl, aminoalkyl, hydroxycarbonylalkyl, alkoxycarbonylalkyl, aminocarbonylalkyl, alkylcarbonylaminoalkyl, sulfonylaminocarbonylalkyl, alkylsulfonylaminocarbonylalkyl, cycloalkylsulfonylaminocarbonylalkyl, arylsulfonylaminocarbonylalkyl, arylalkylsulfonylaminocarbonylalkyl, phenylalkyl, tetrazolylalkyl, pyridinylalkyl, or pyrimidinylalkyl; or R² is phenyl, pyridinyl, pyrimidinyl, pyridazinyl, or pyrazolyl and is optionally substituted with one or more substituents each independently being hydroxyl, halo, alkyl, alkoxy, cyano, phenyl, hydroxycarbonyl, alkoxycarbonyl, aminocarbonyl, alkylcarbonylaminoalkyl, alkylaminocarbonyl, or hydroxyphenylpiperazinyl.

9. The compound of claim 6, wherein R² is halo, —(CH₂)₂OH, —CH(CH₂OH)₂, —(CH₂)₂COOH, —(CH₂)₂CONH₂, —CH₂COOH, —CH₂COOCH₃, —CH₂—CO—NH—CH₂—COOH, —CH(CH₃)COOH, —C(CH₃)₂—COOH, CH(CH₃)COOCH(CH₃)₃, CH(C₂H₅)COOH, —CH₂CH₂NH₂, —CH₂CONH₂, —CH₂CONHCH₃, —CH₂CONHC₂H₅, —CH(C₂H)CH₂OH, —CH₂CO—NH—SO₂CH₃, —CH₂CO—NH—SO₂C₂H₅, —CH₂CO—NH—SO₂CH(CH₃)₂, —(CH₂)₂—NH—SO₂—CH₃, —CH₂CO—NH—SO₂CH₂CH₂CH₃, (cyclopropyl)SO₂—NH—COCH₂—, phenyl-SO₂—NH—COCH₂—, benzyl-SO₂—NH—COCH₂—, tetrazolylmethyl, tetrazolylethyl, or pyridinylmethyl; or R² is phenyl, pyridinyl, pyrimidinyl, pyridazinyl, or pyrazolyl and is optionally substituted with one or two substituents each independently being hydroxyl, halo, alkyl, alkoxy, cyano, phenyl, hydroxycarbonyl, aminocarbonyl, methylaminocarbonyl, or hydroxyphenylpiperazinyl.

10. The compound of claim 1, wherein R³ is H; and R⁴ is H, hydroxycarbonylalkyl, or alkoxycarbonylalkyl.

11. The compound of claim 1, wherein X is N, Y is N, Z is C.

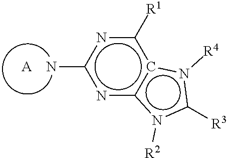

12. The compound of claim 1, wherein X is CR⁵, Y is C, Z is N.

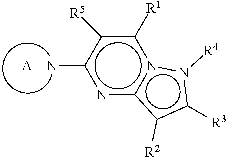

13. The compound of claim 12, wherein R⁵ is H.

14. The compound of claim 1, wherein Ring A is

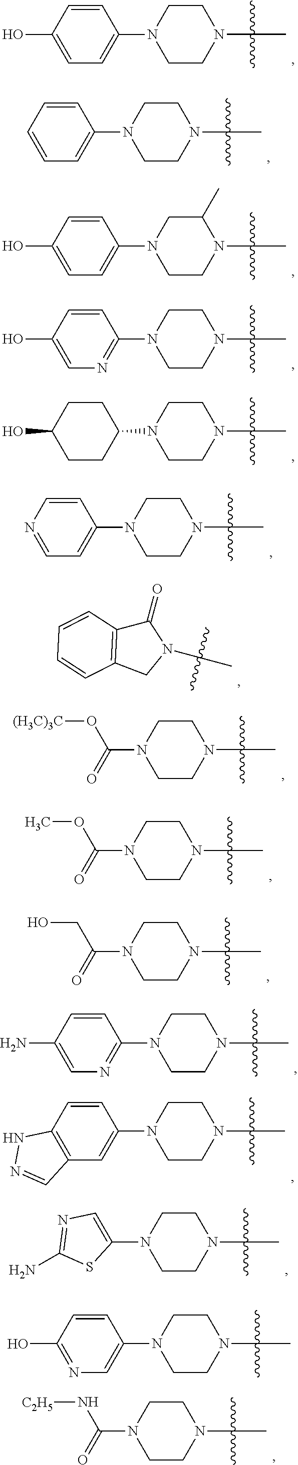

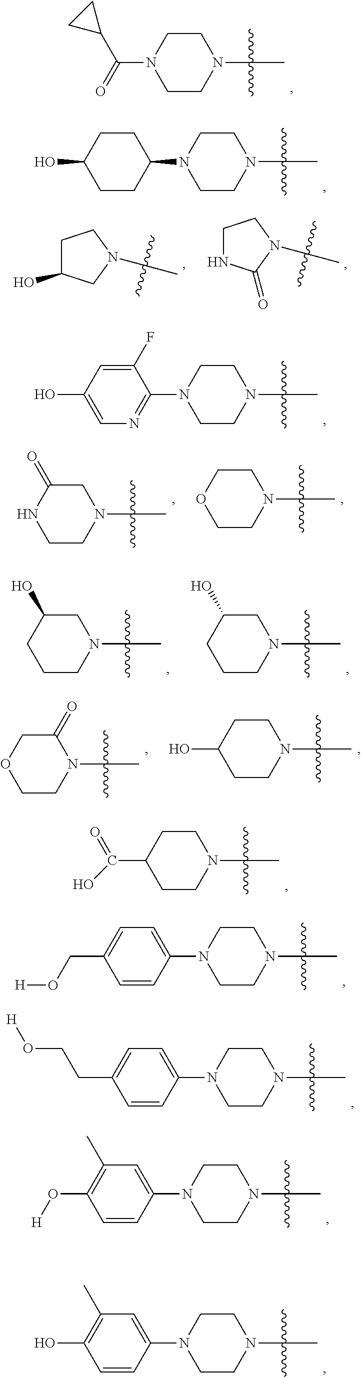

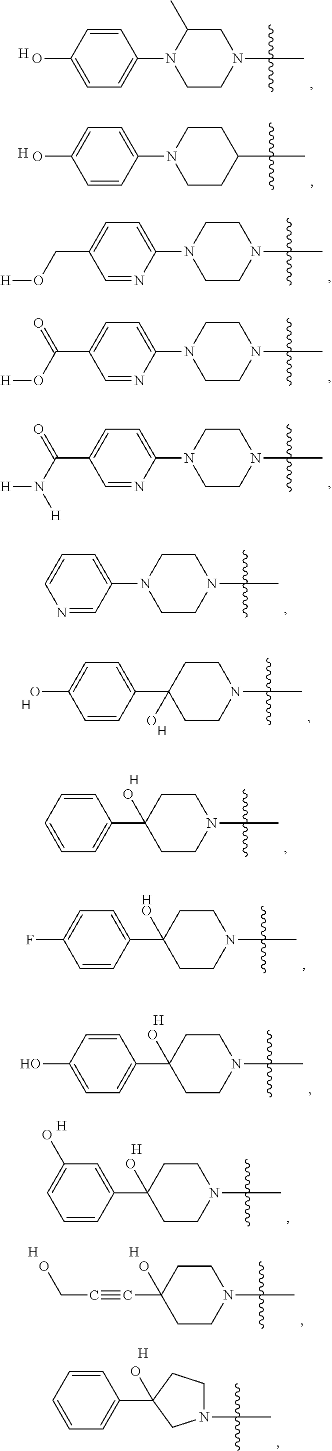

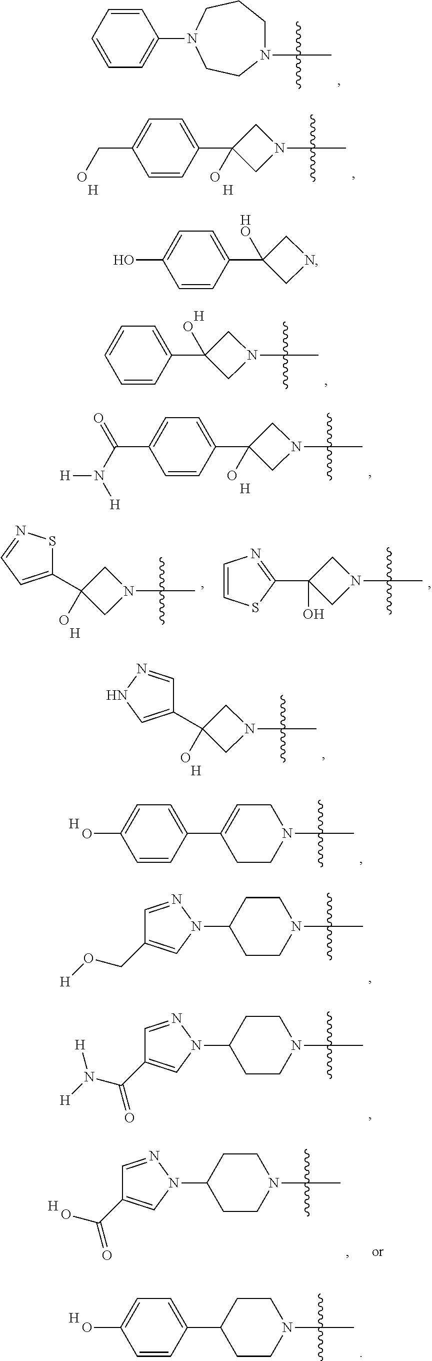

15. The compound of claim 1, wherein the compound is

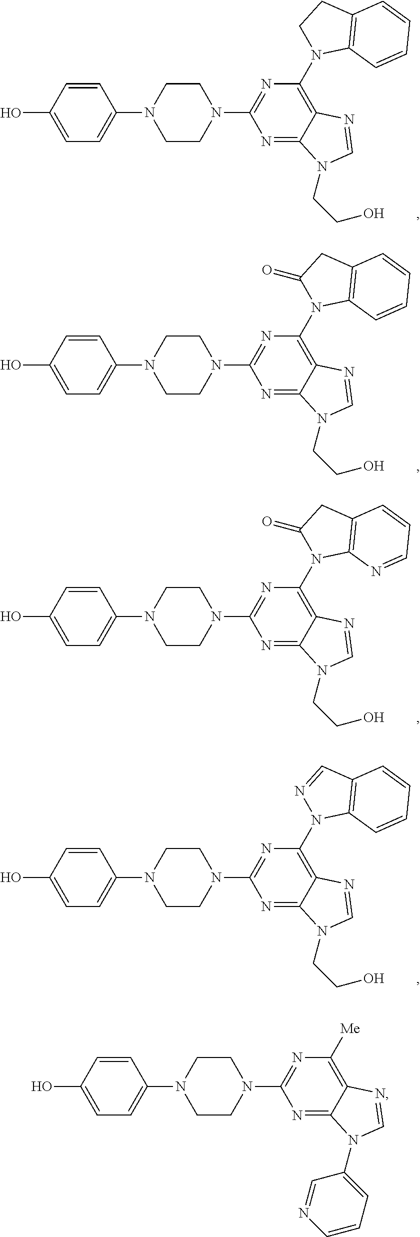

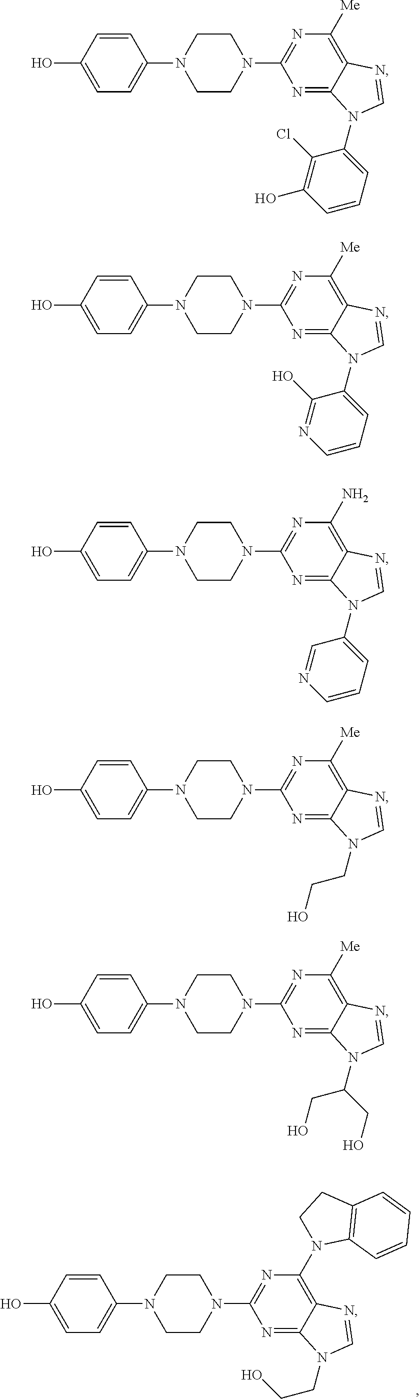

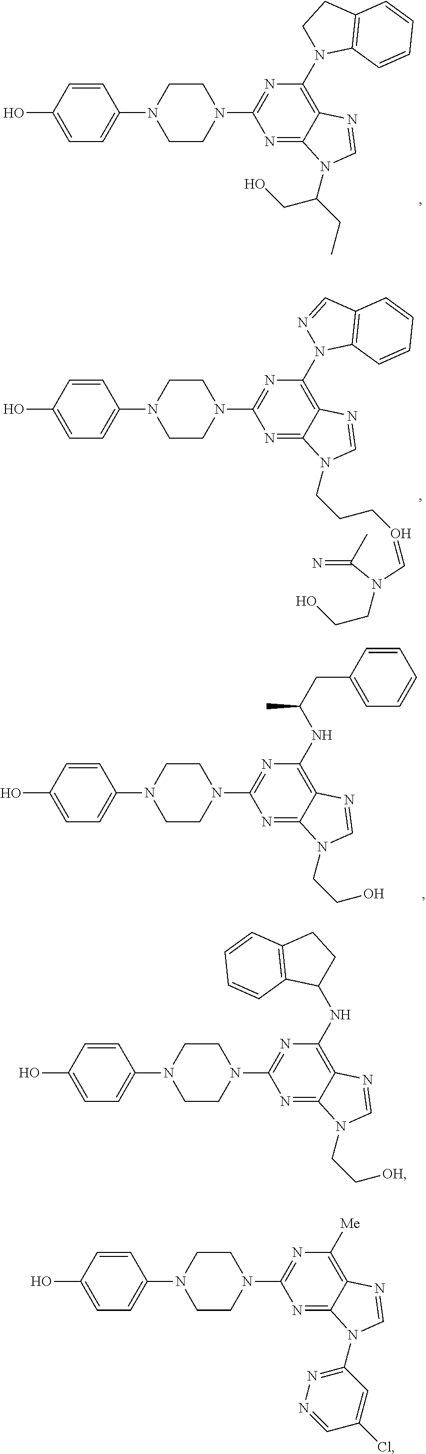

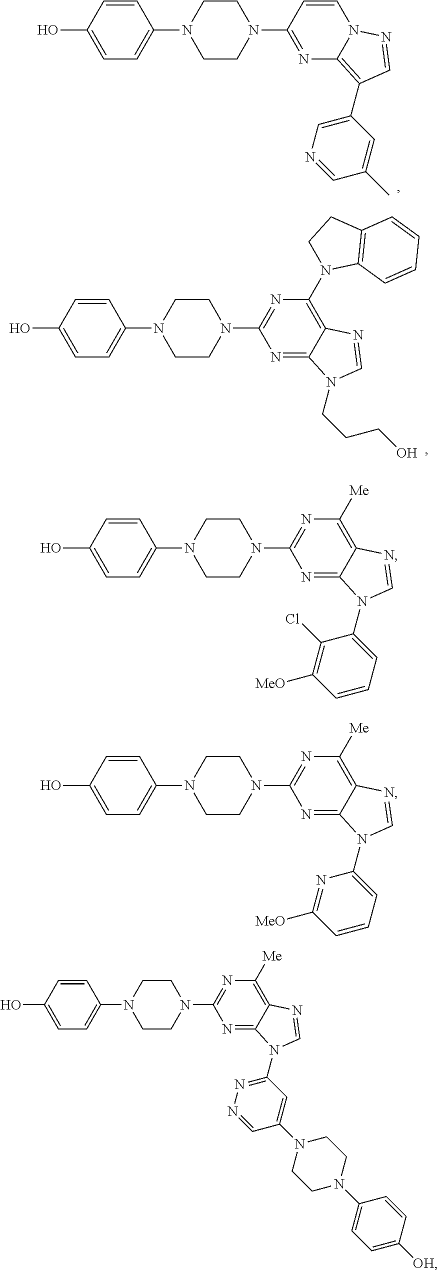

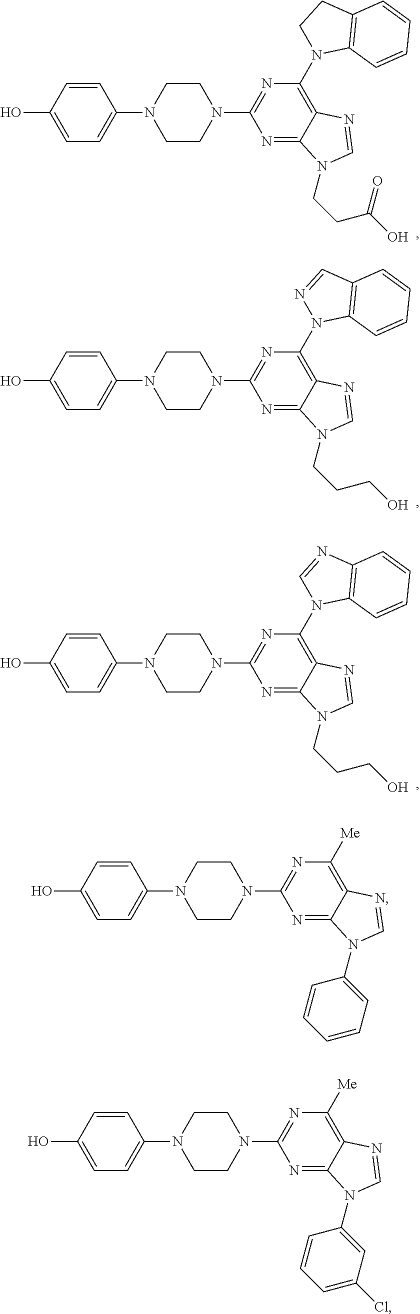

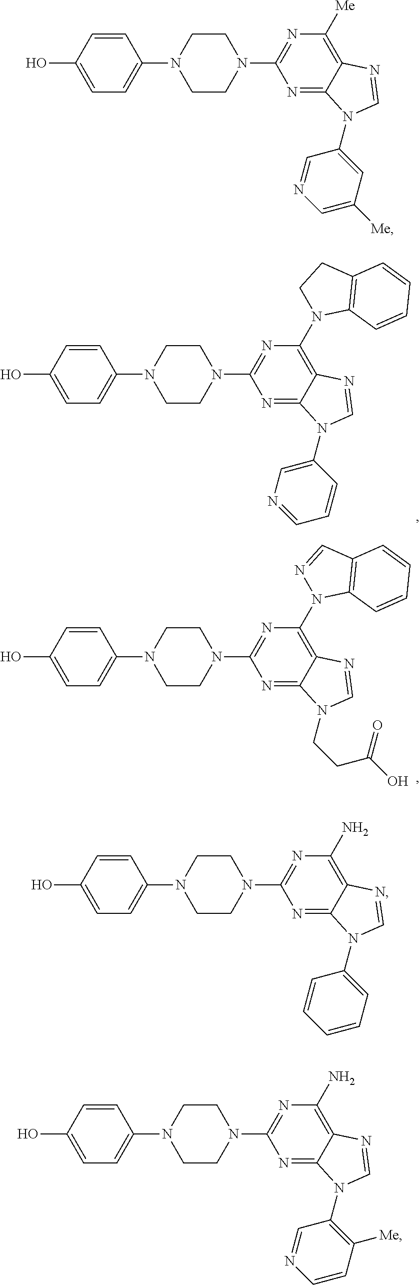

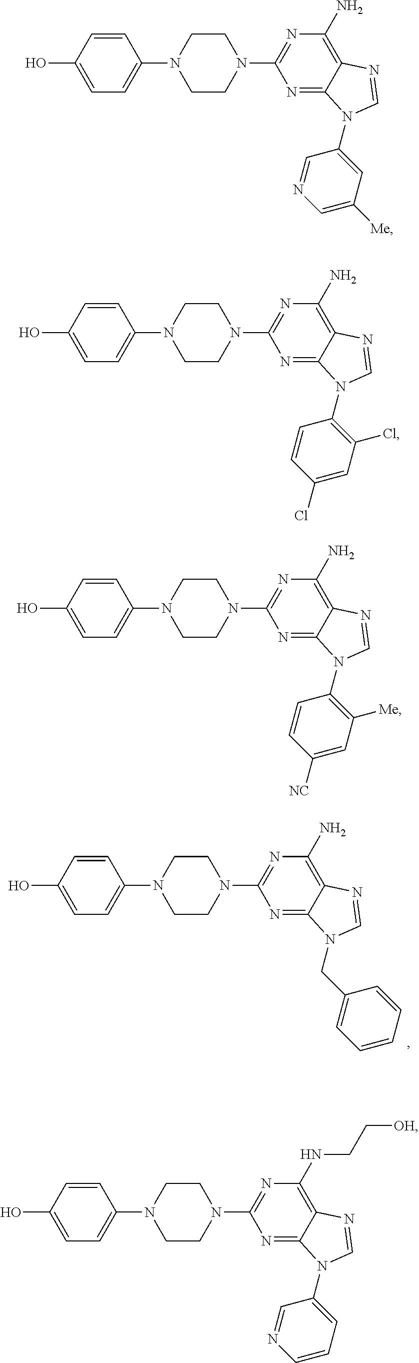

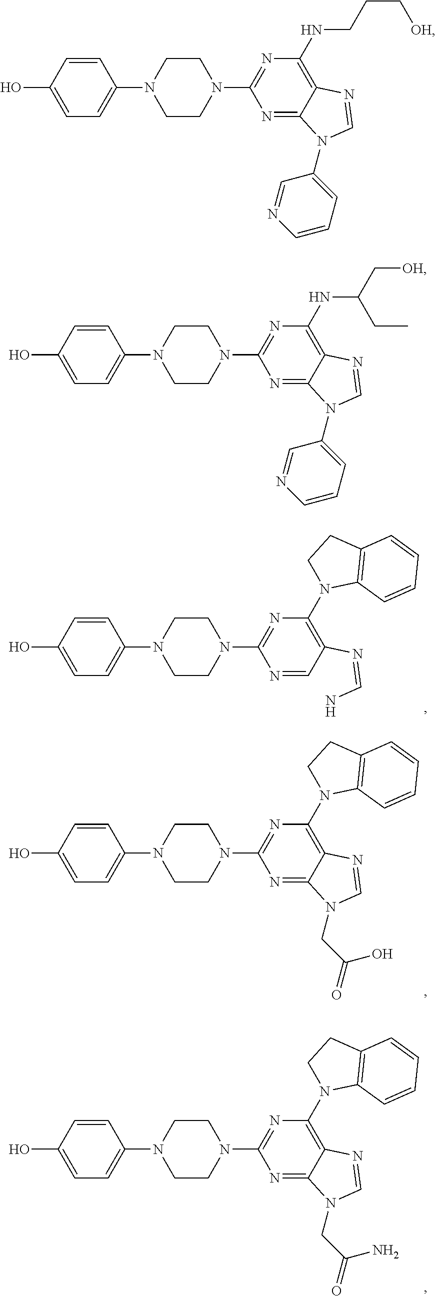

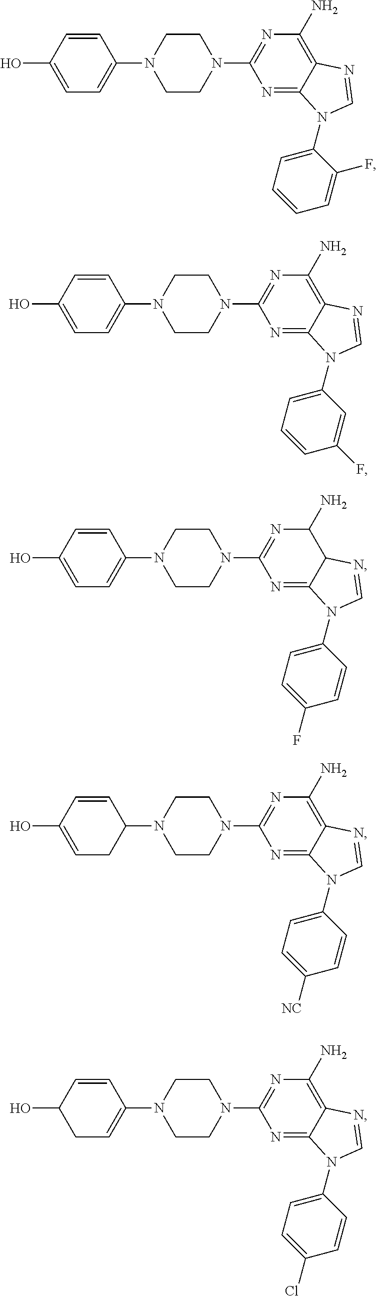

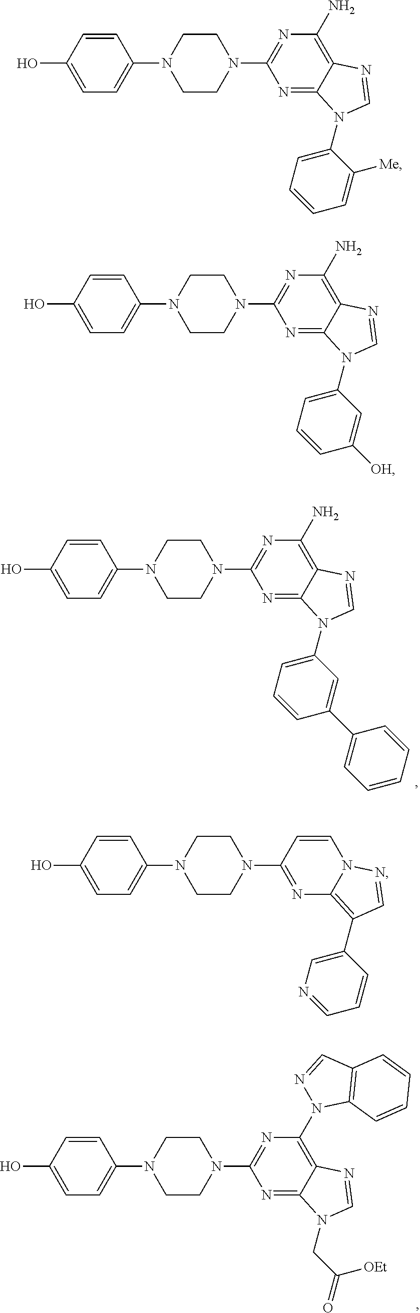

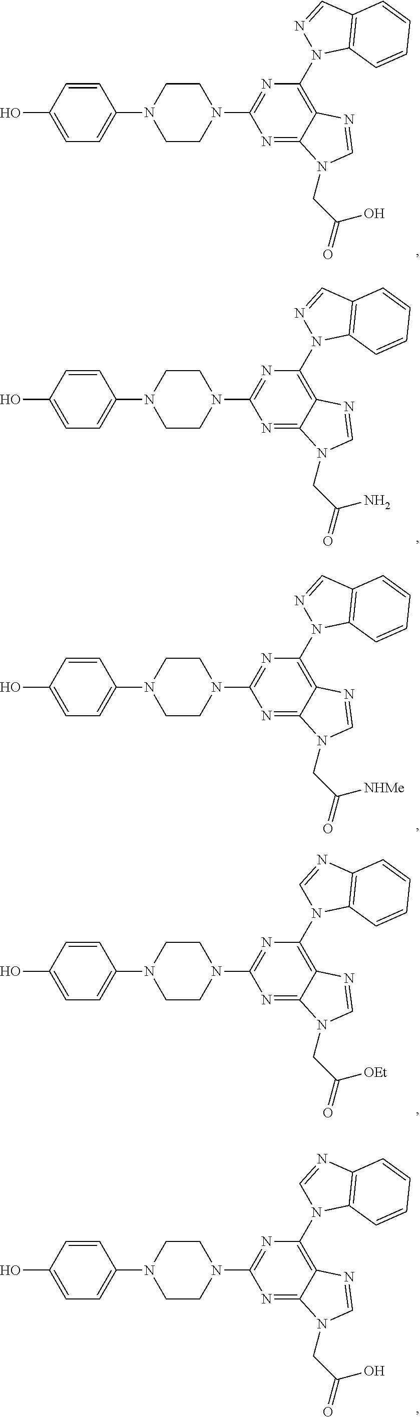

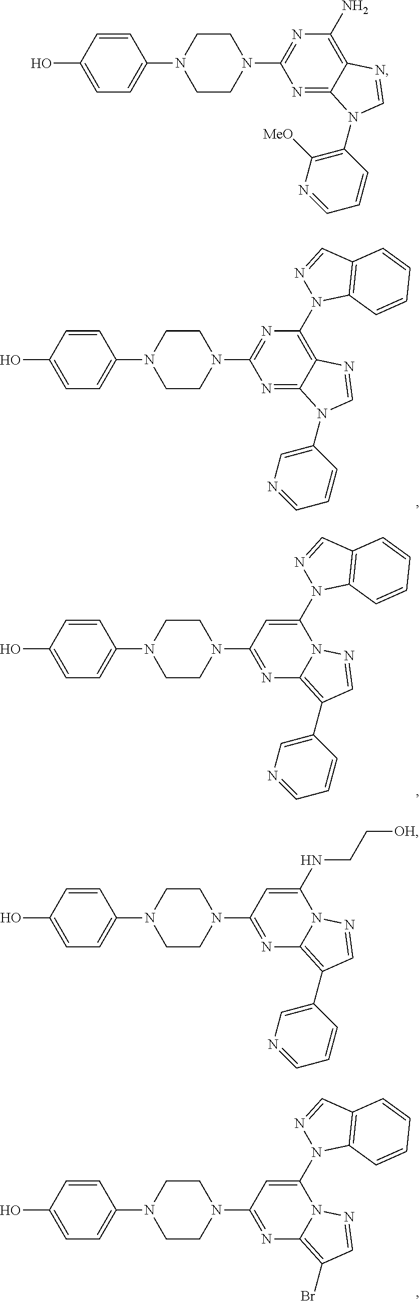

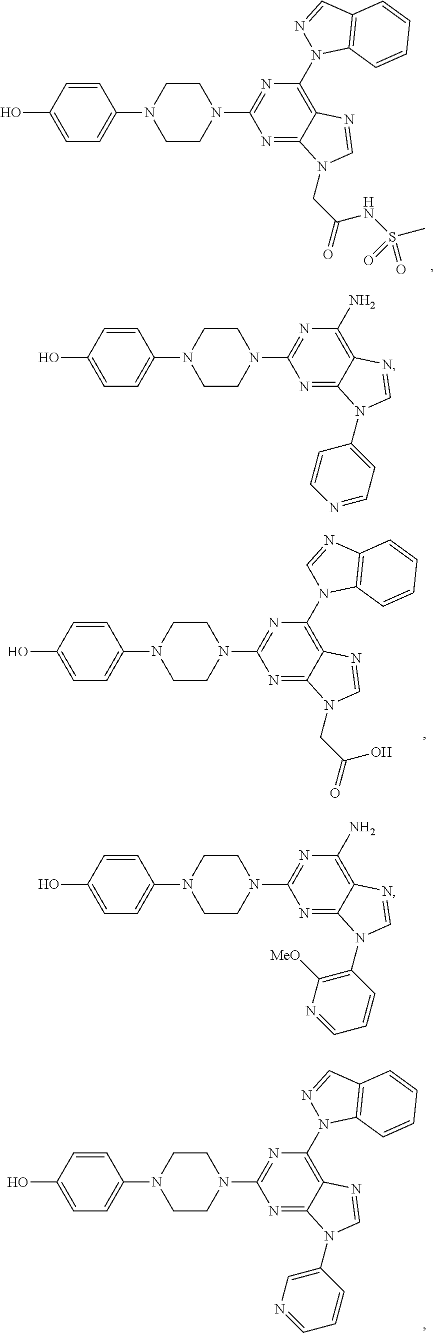

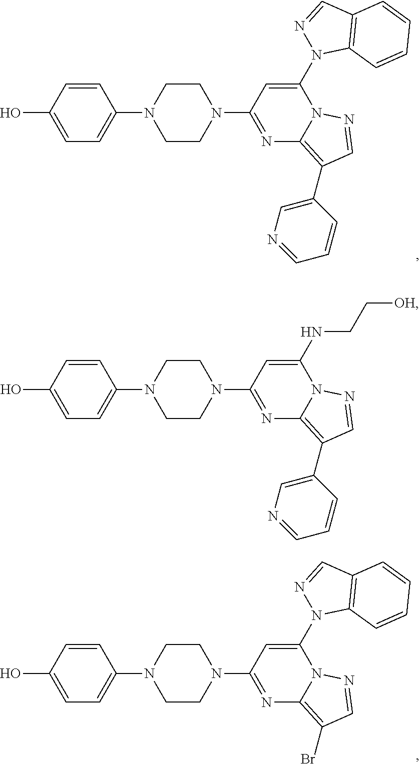

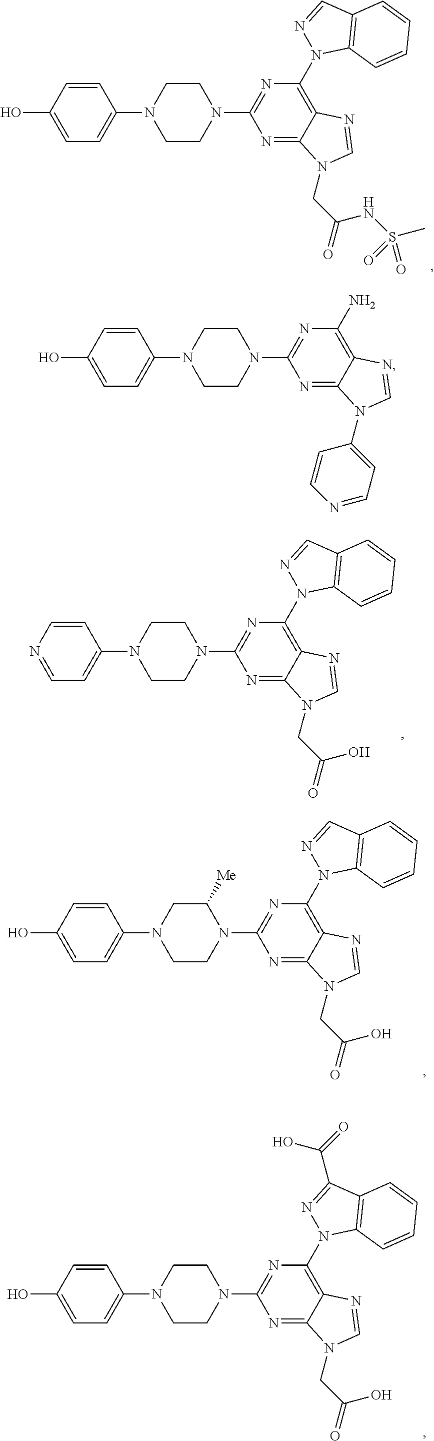

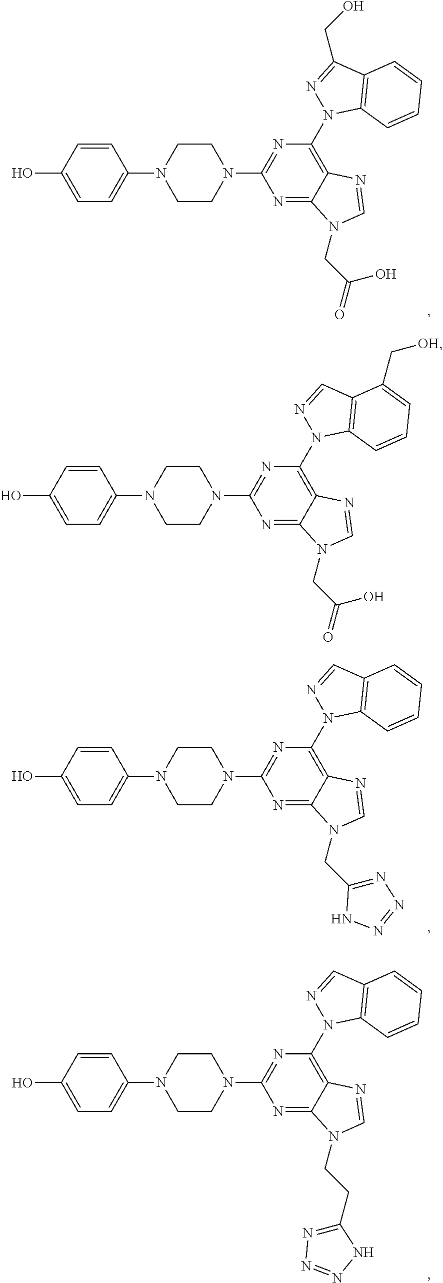

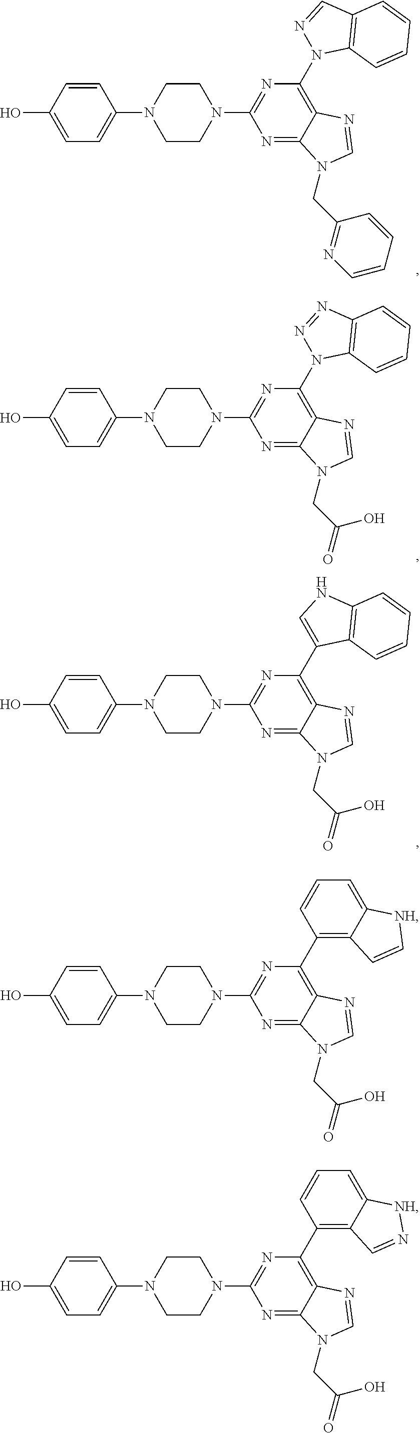

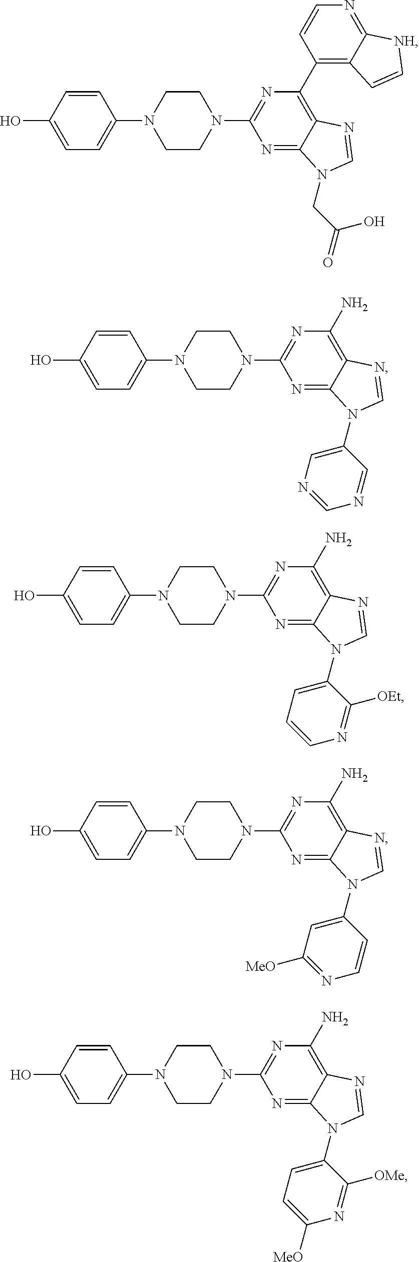

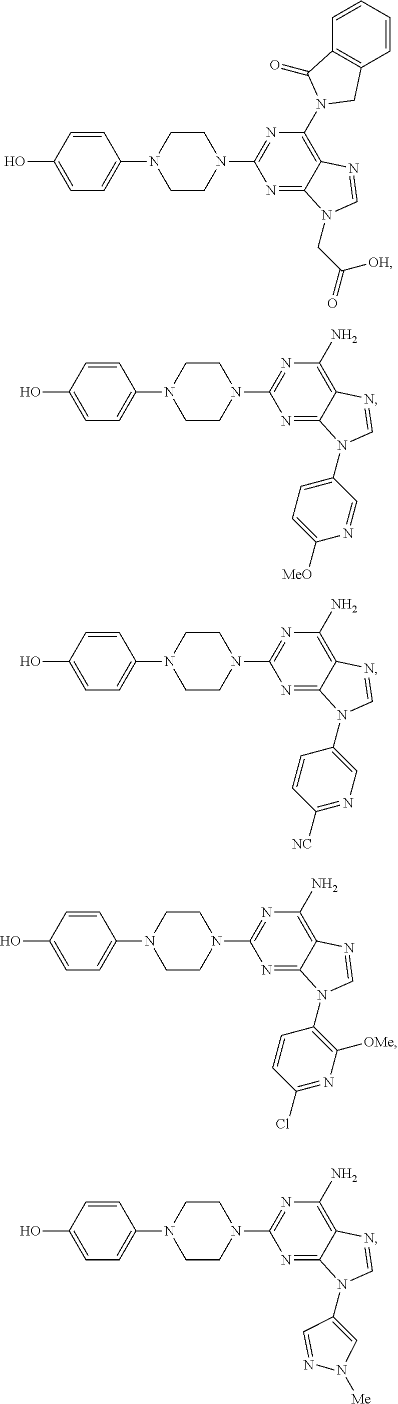

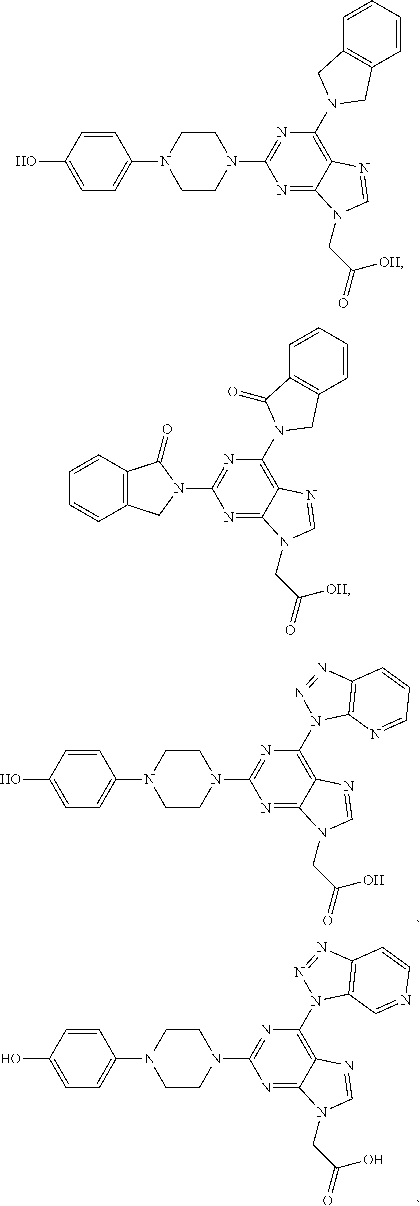

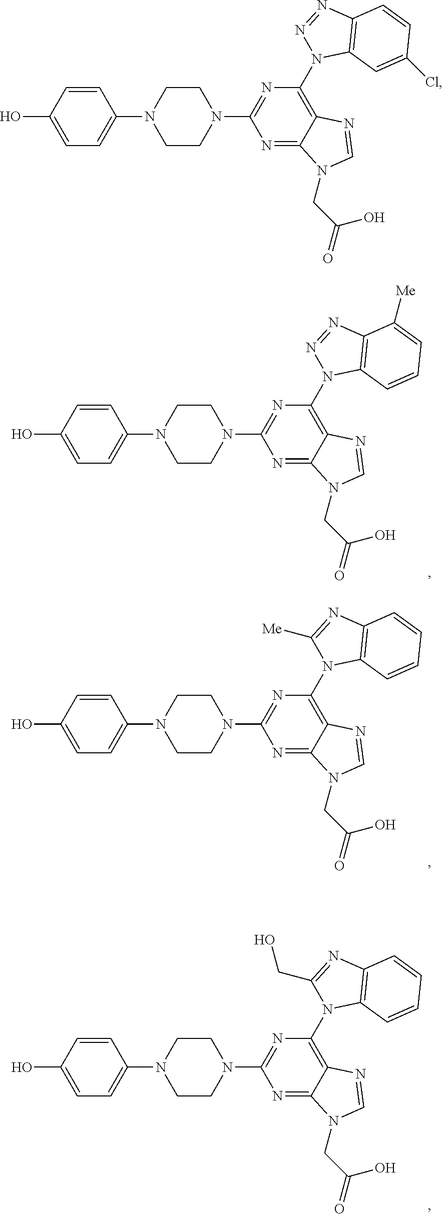

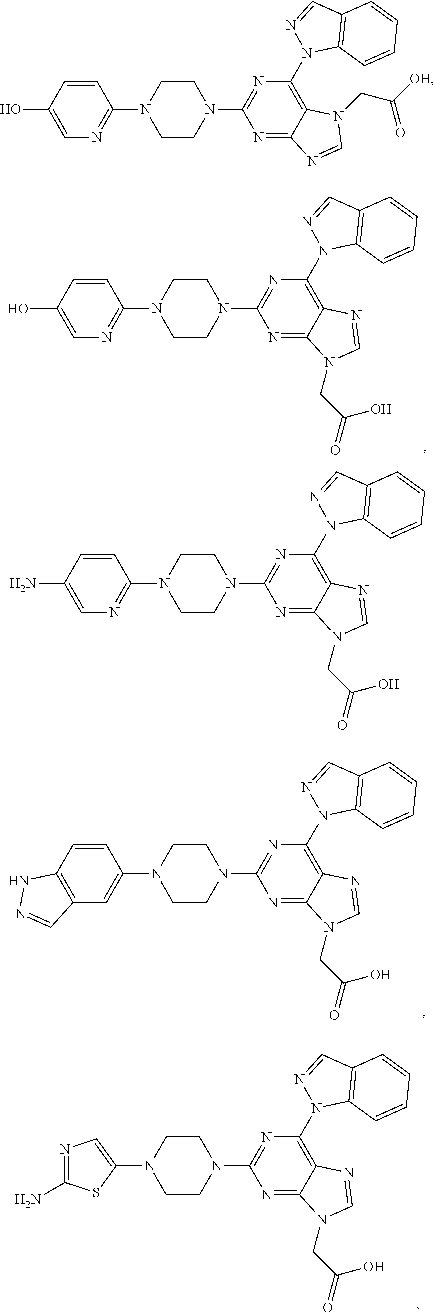

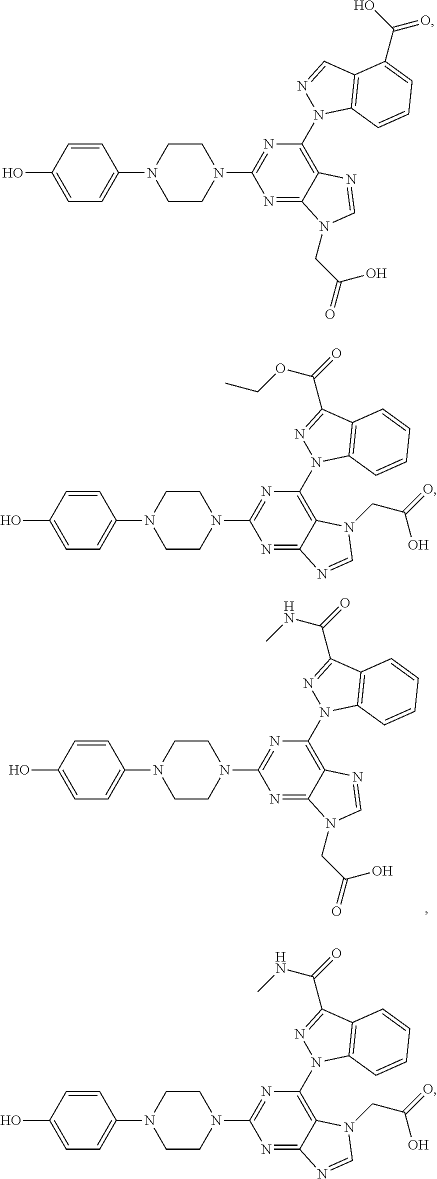

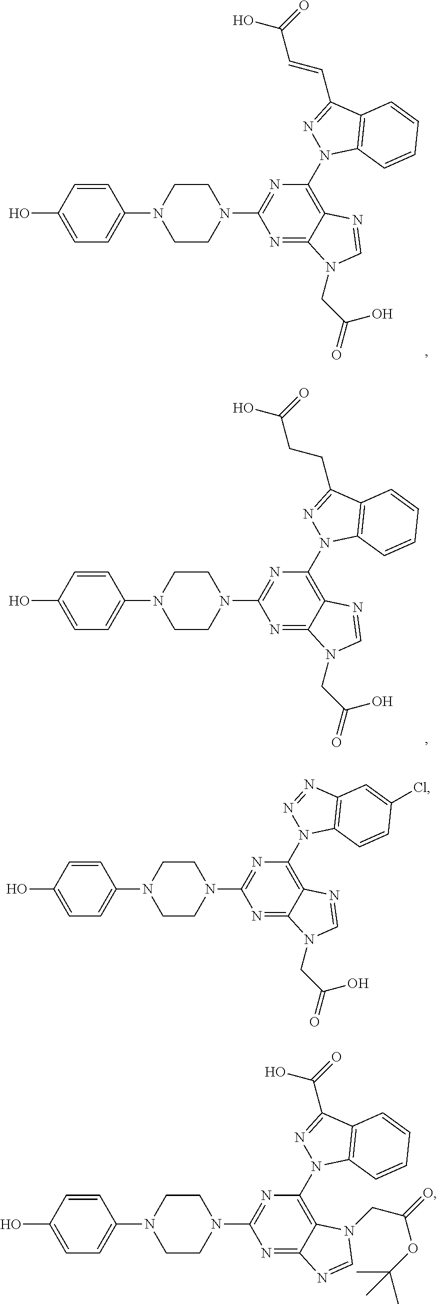

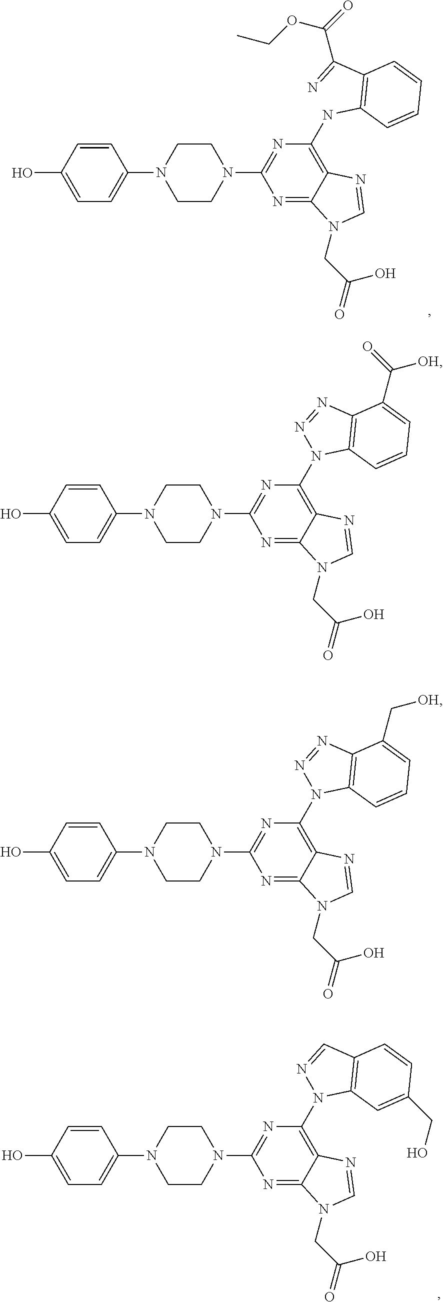

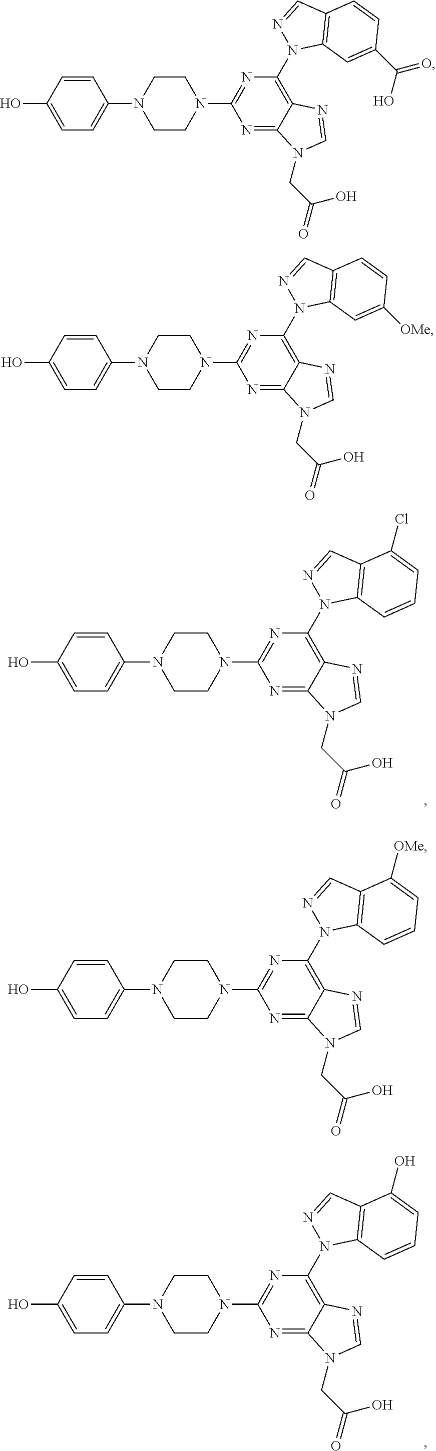

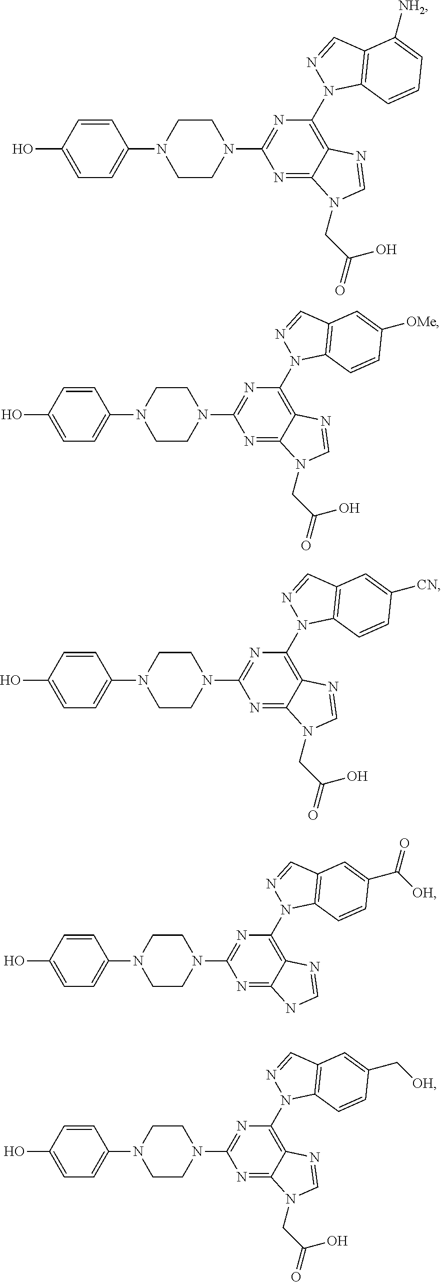

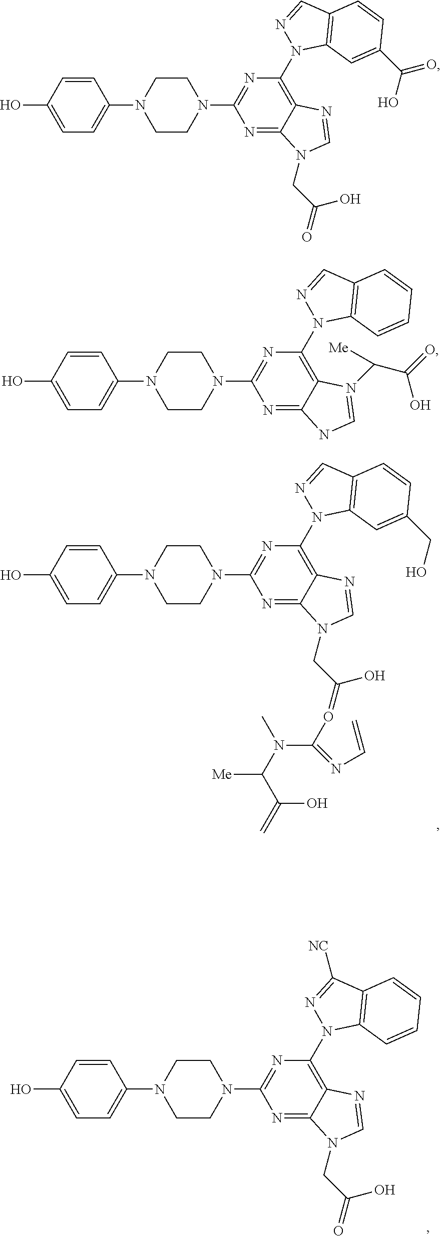

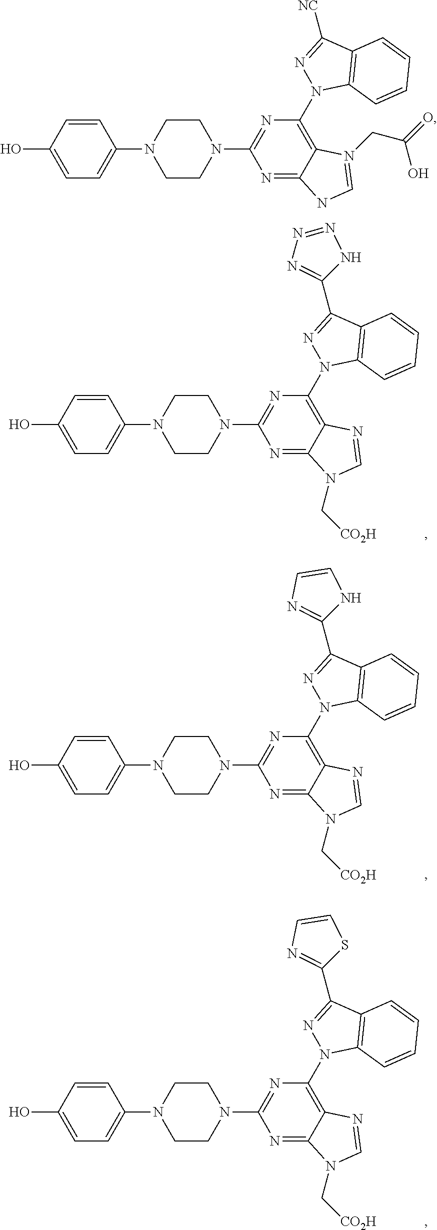

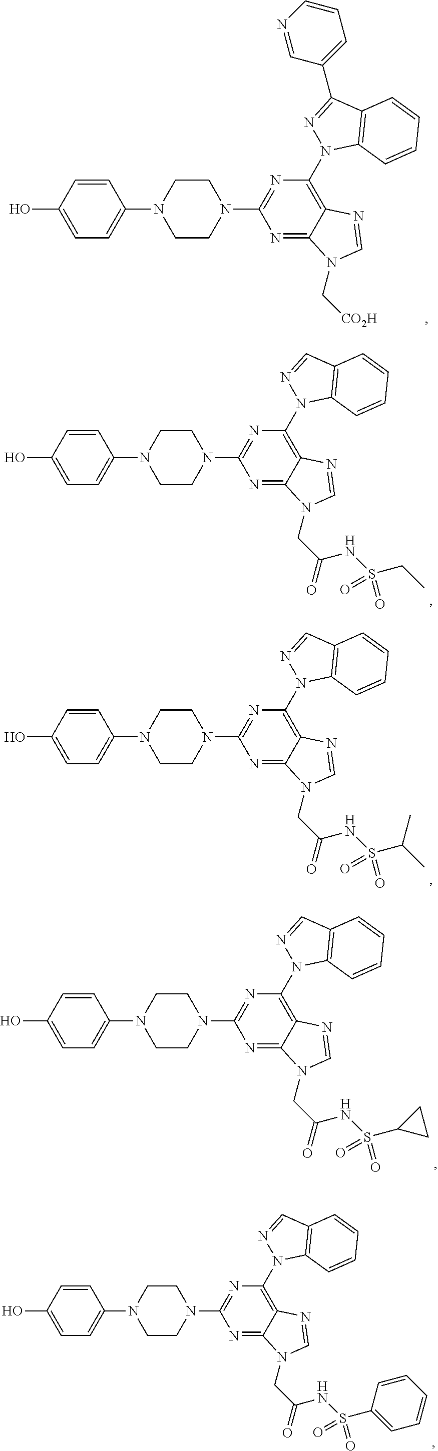

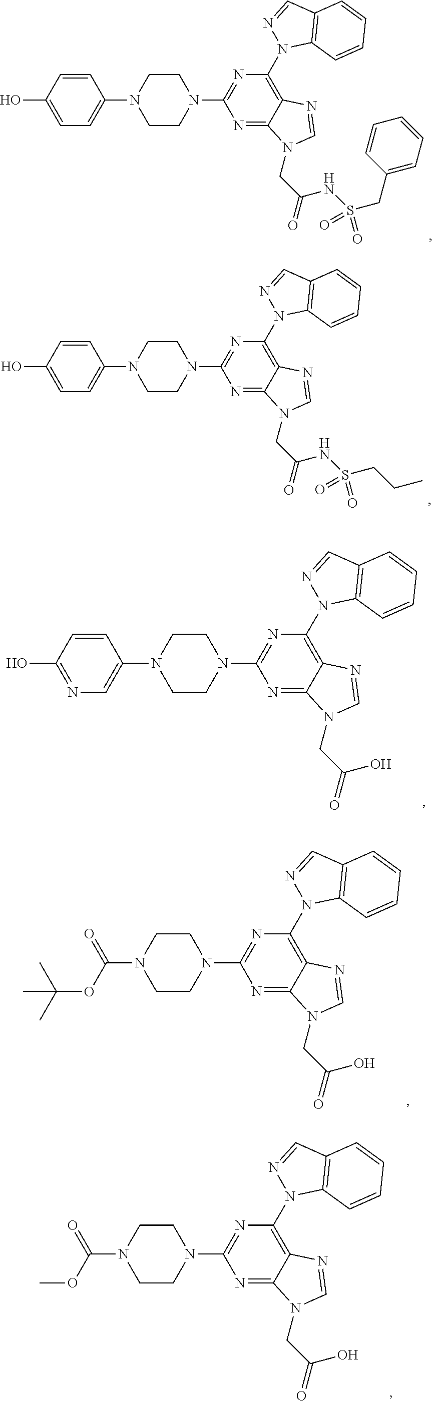

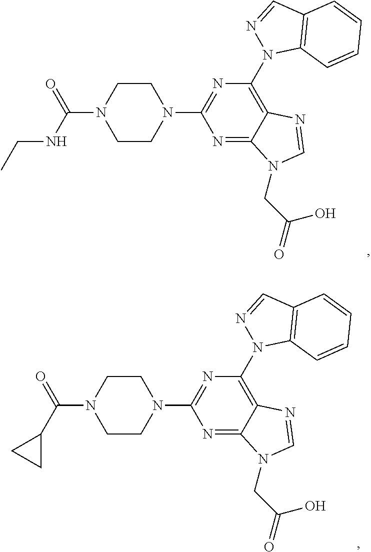

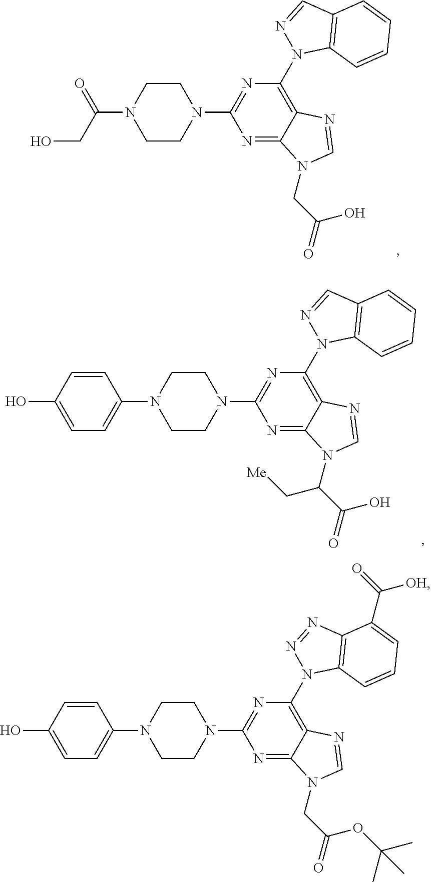

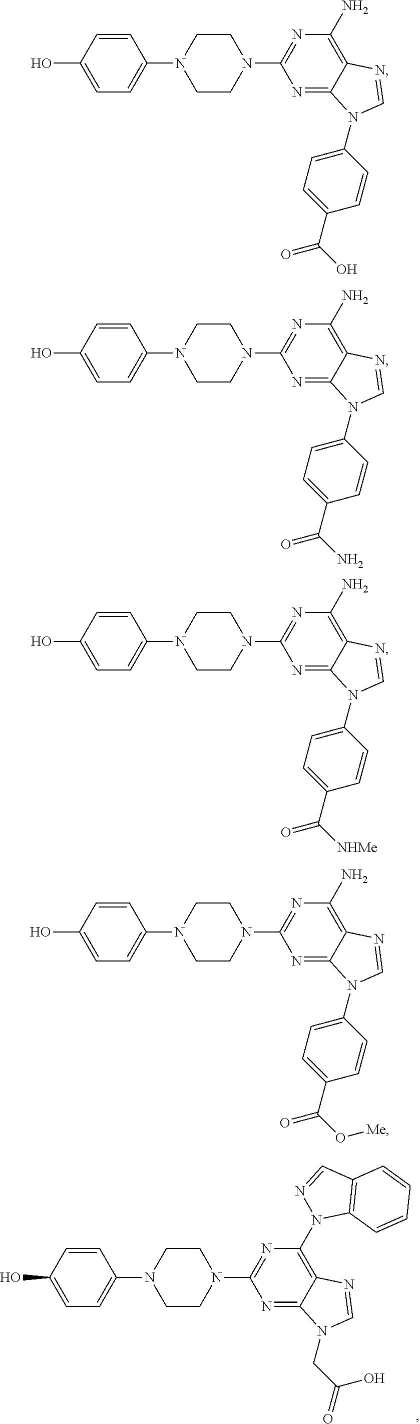

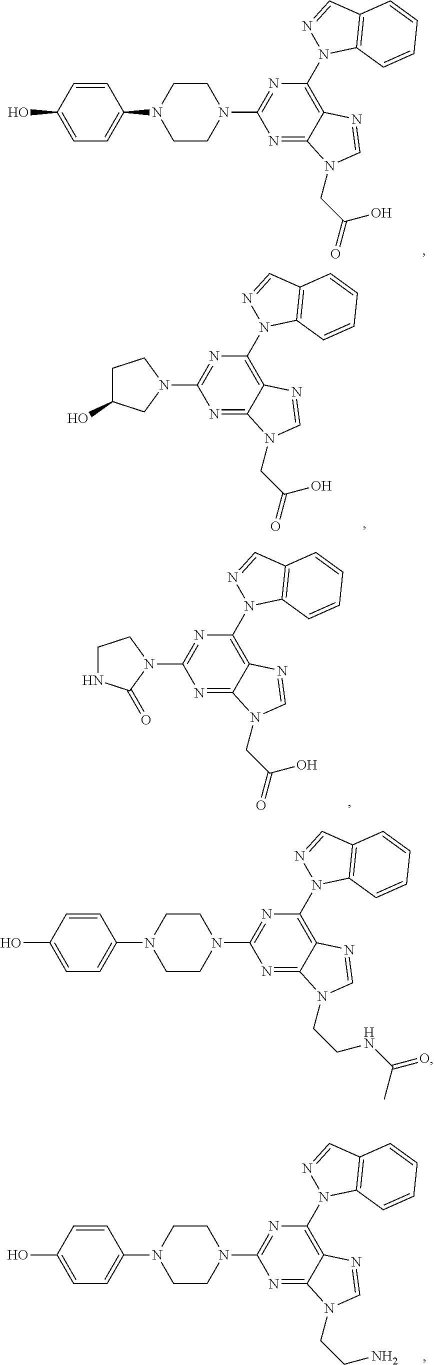

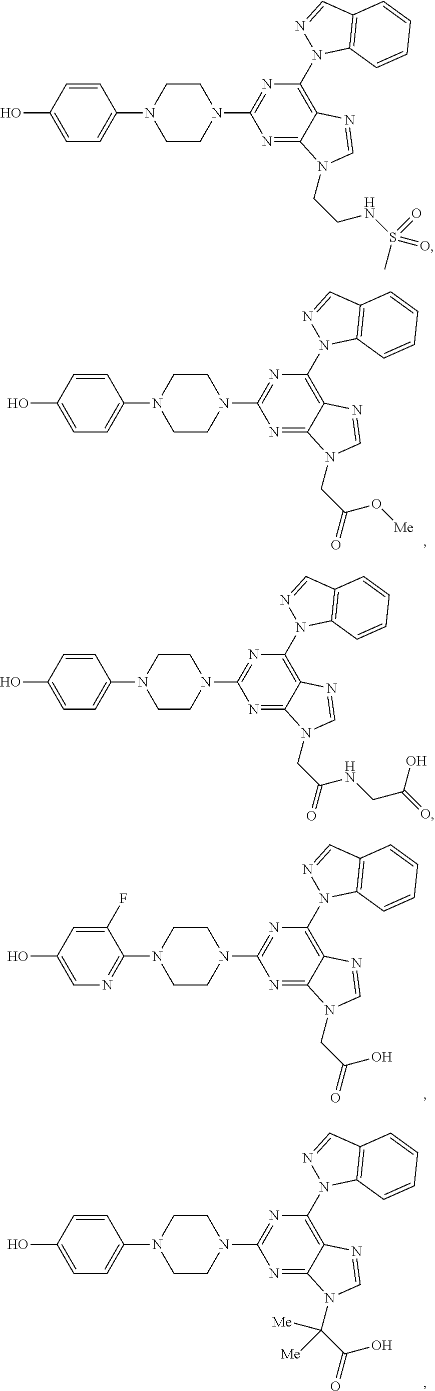

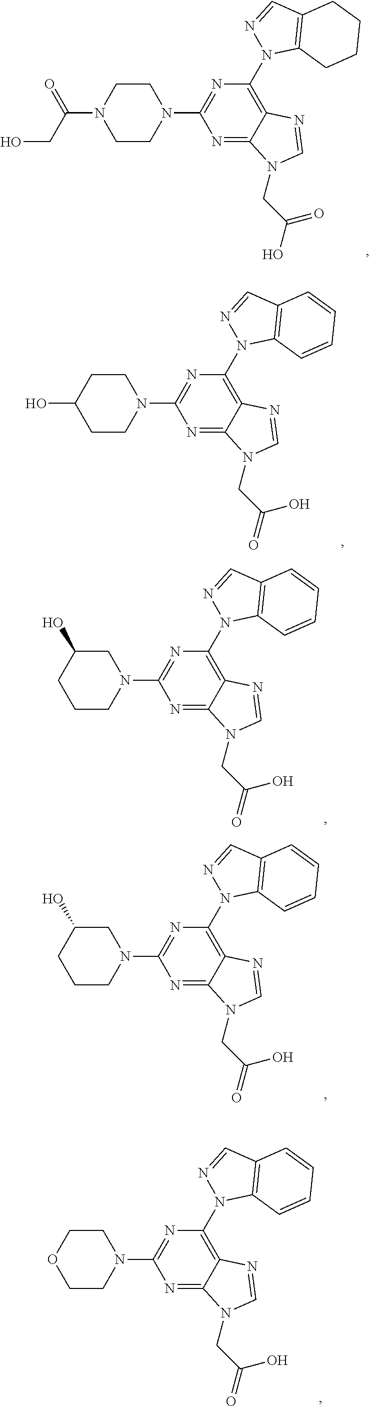

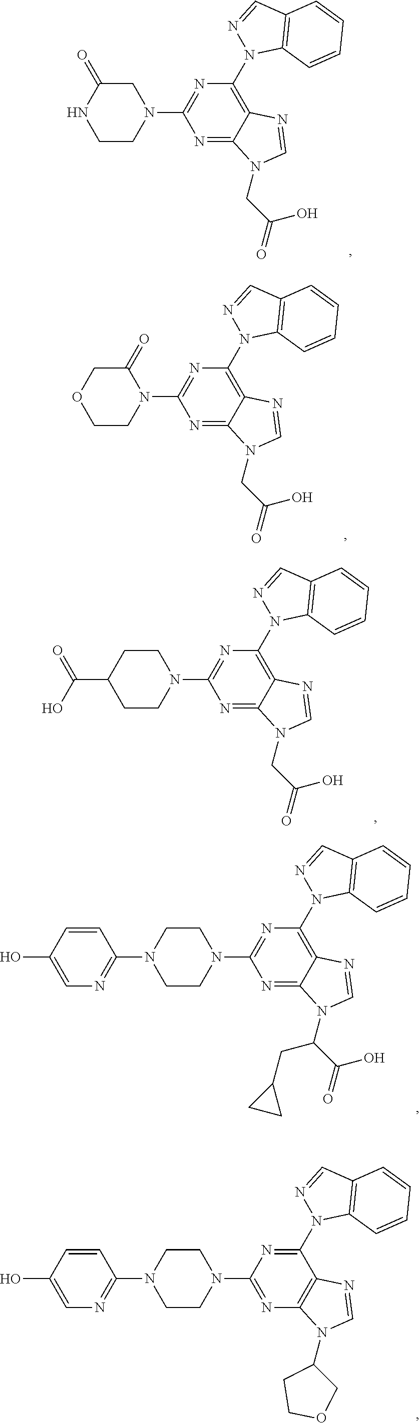

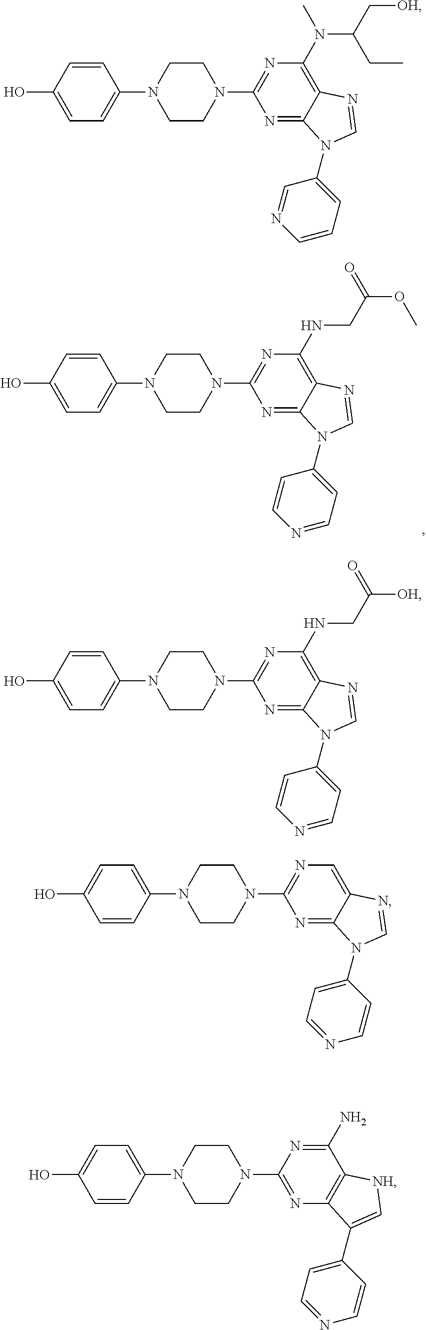

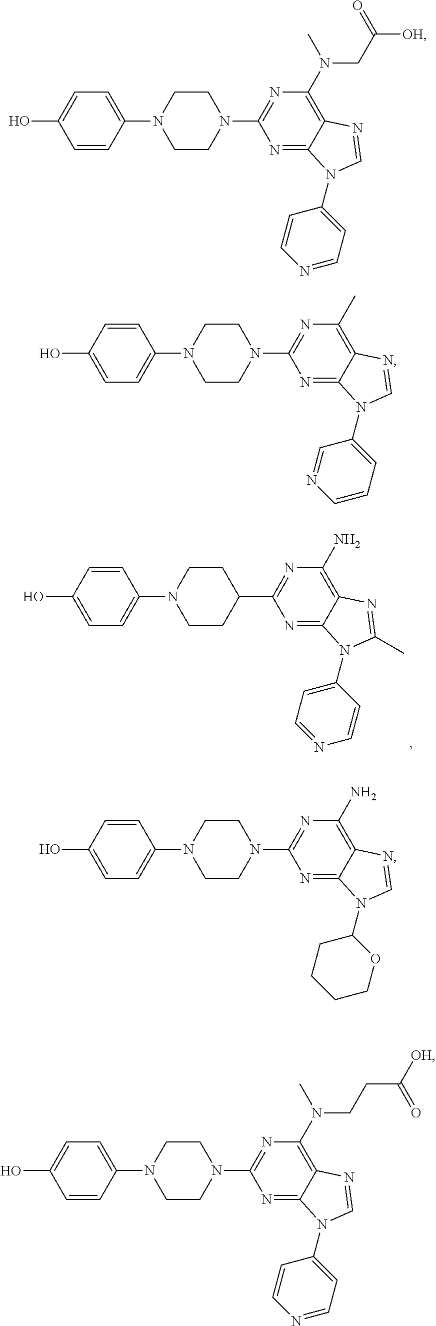

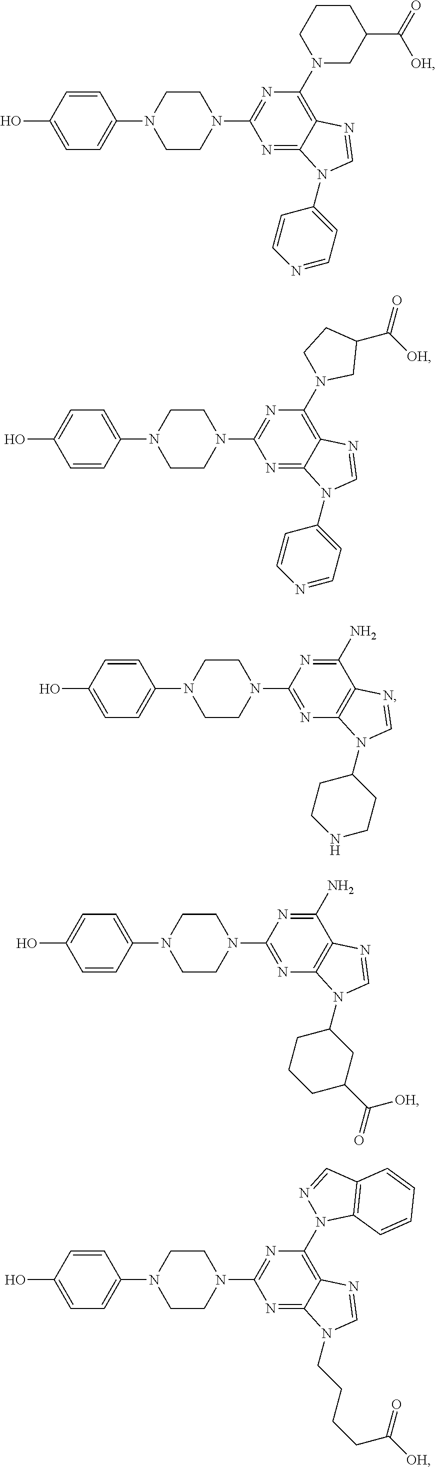

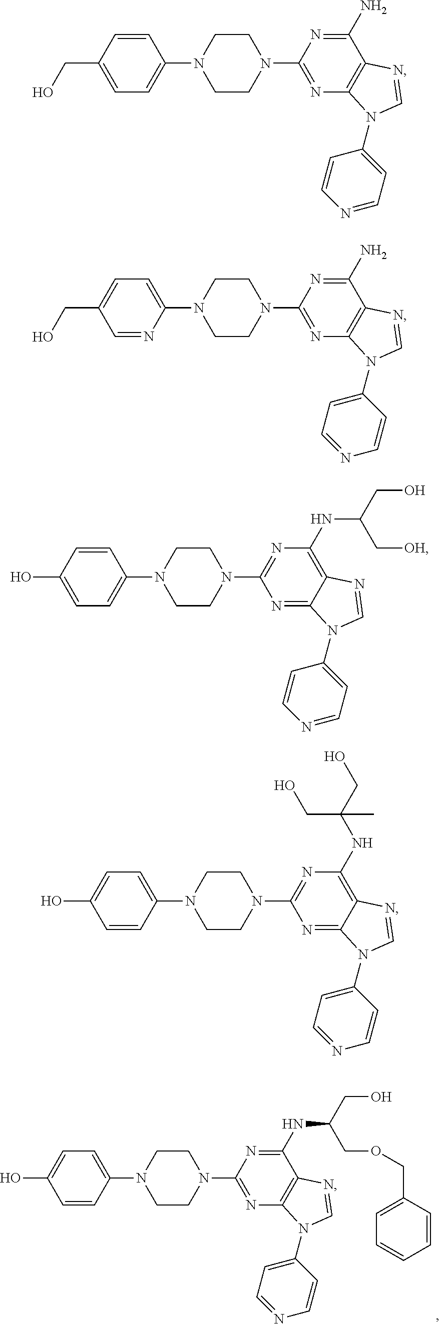

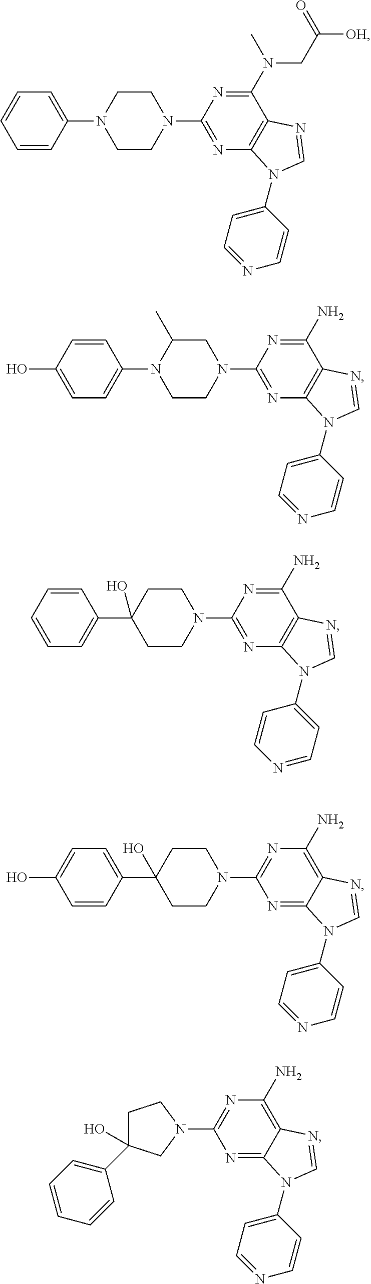

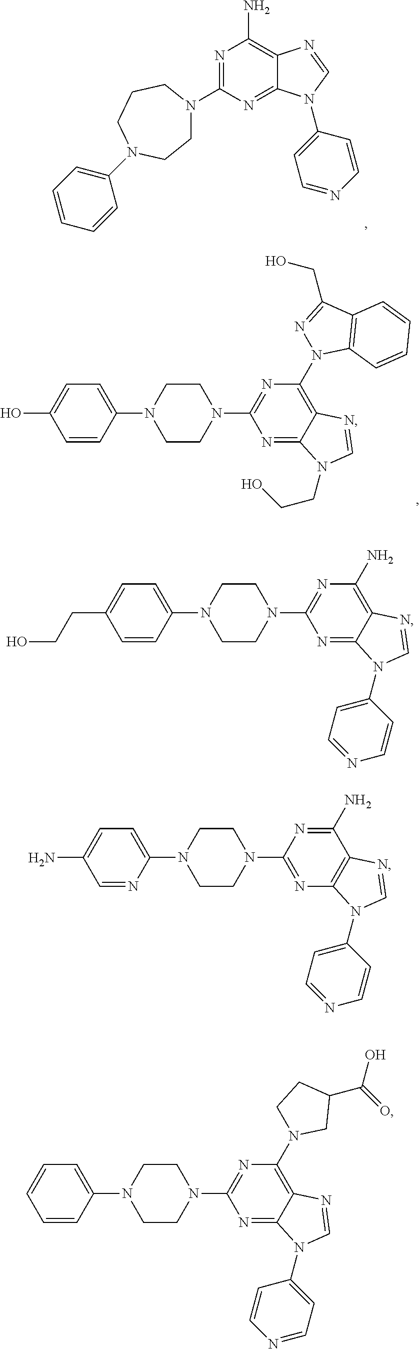

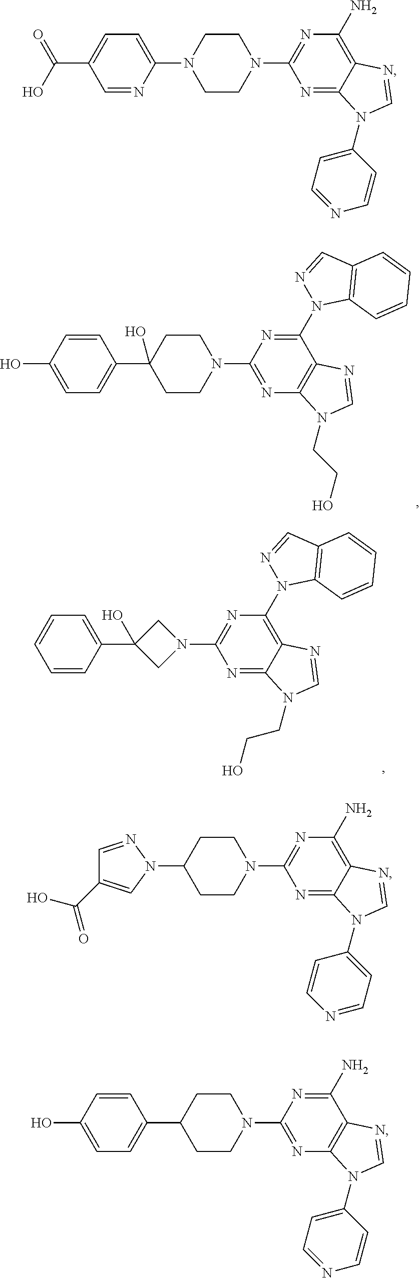

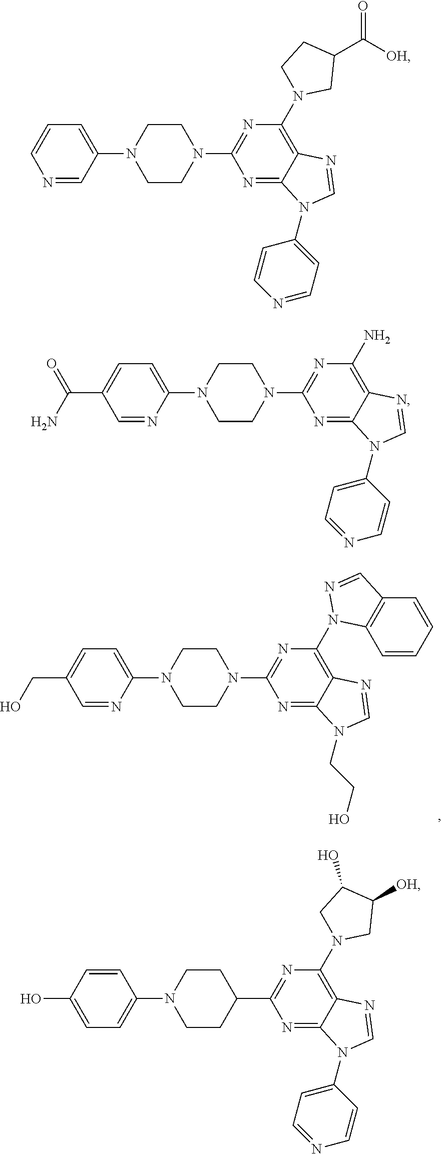

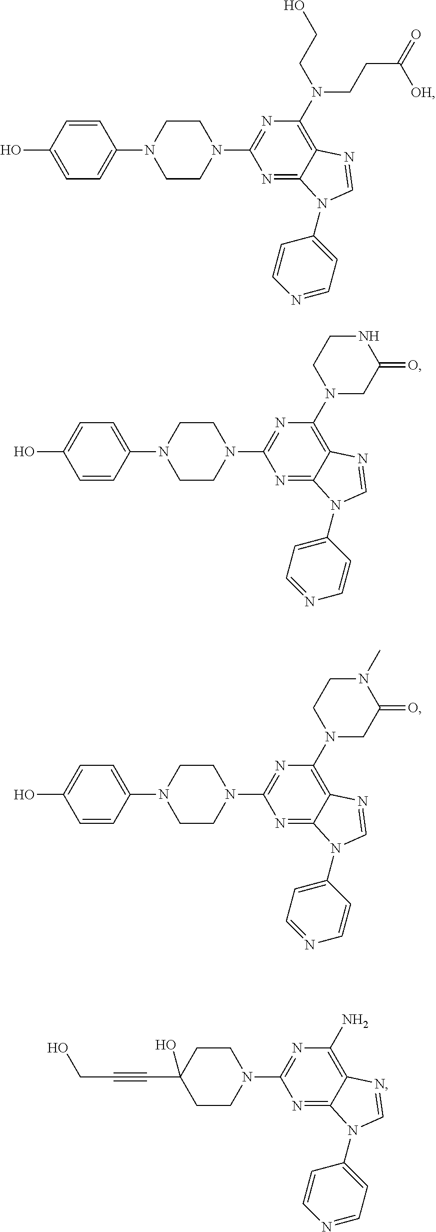

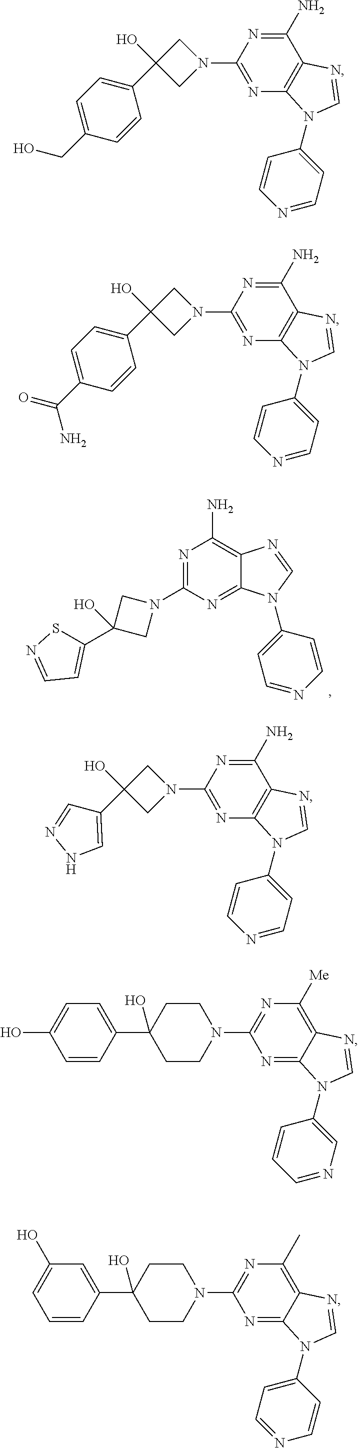

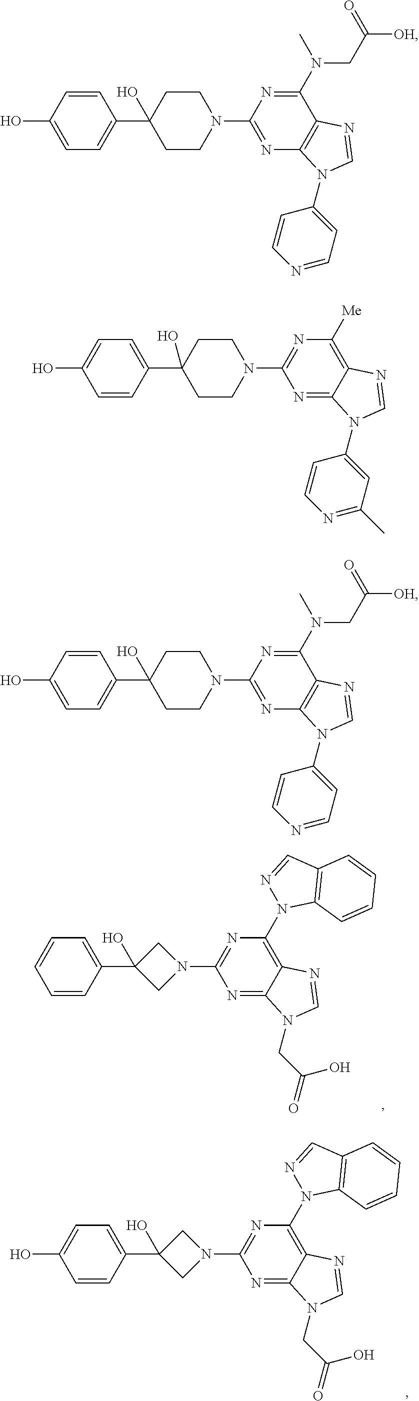

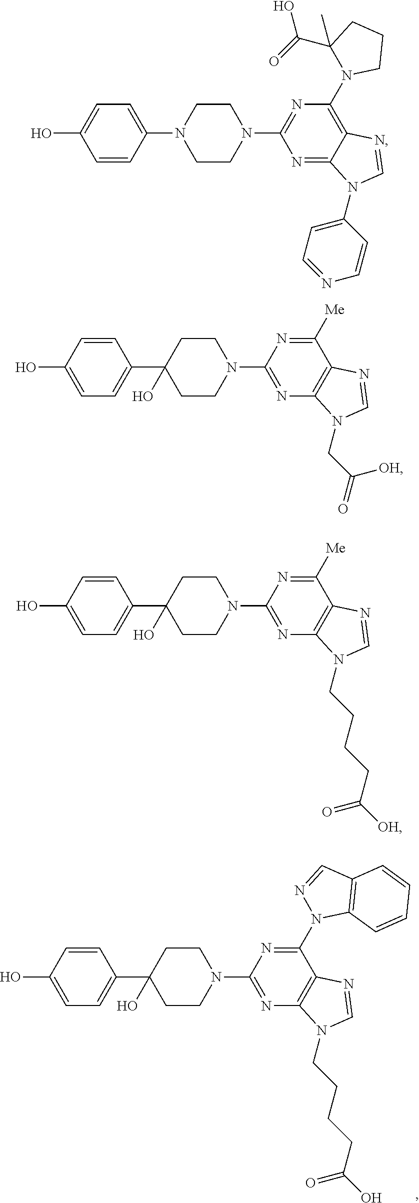

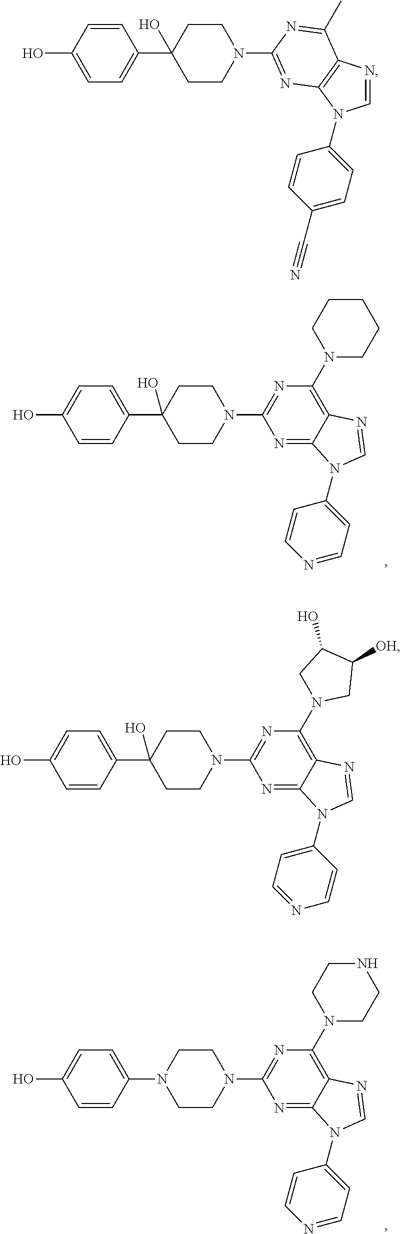

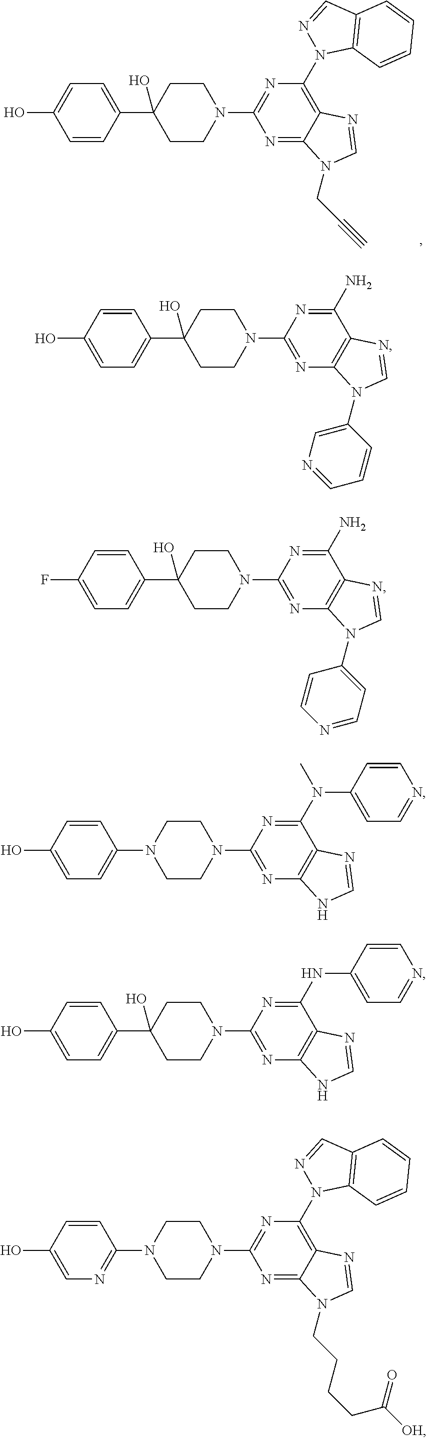

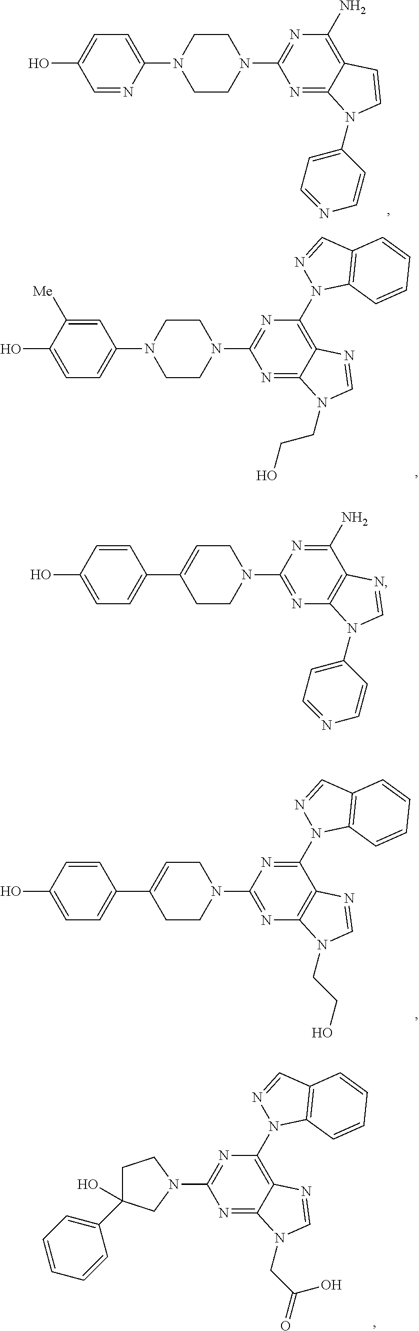

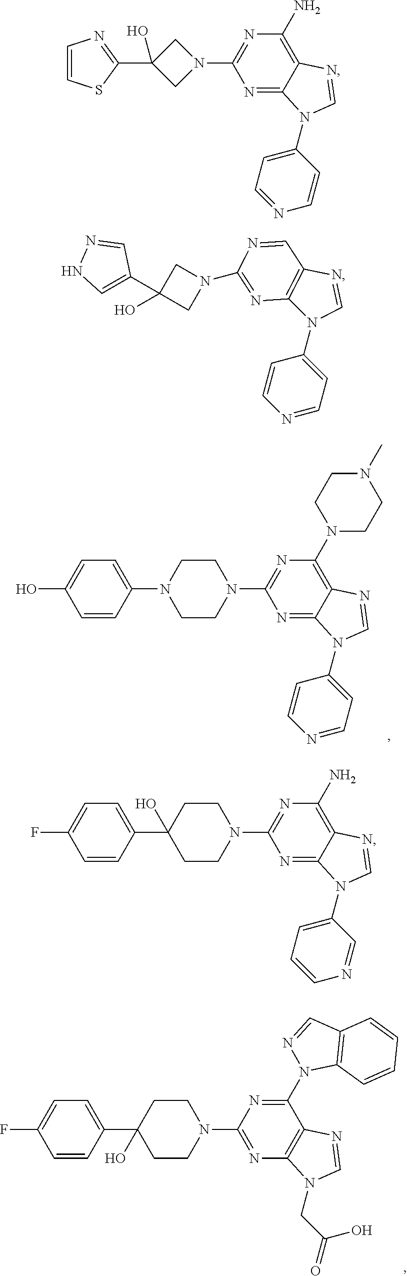

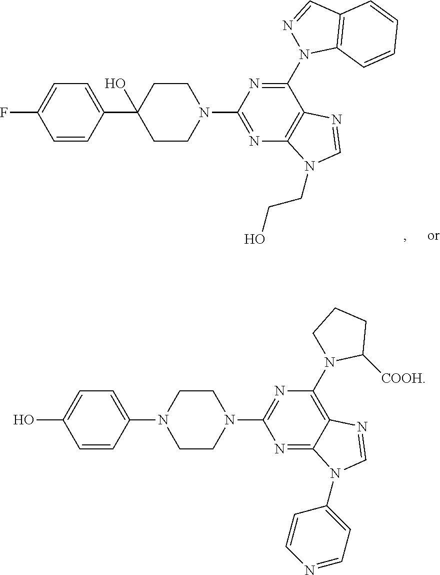 [SMILES: OCCn1cnc2c(-n3ncc4ccccc43)nc(N3CCC(O)(c4ccc(F)cc4)CC3)nc21 ; O=C(O)C1CCCN1c1nc(N2CCN(c3ccc(O)cc3)CC2)nc2c1ncn2-c1ccncc1]

16. A pharmaceutical composition comprising a compound of claim 1 and a pharmaceutically acceptable carrier.

17. The pharmaceutical composition of claim 16, further comprising a second therapeutic agent different from the compound of claim 1.

18. A method for treating a disorder mediated by macrophage migration inhibitory factor in a subject, comprising administering to the subject in need thereof a compound of claim 1.

19. The method of claim 18, wherein the disorder is an immunoinflammatory disease or cancer.

20. The method of claim 19, wherein the immunoinflammatory disease is asthma or rheumatoid arthritism; the cancer is gastric cancer, pancreatic cancer, melanoma, hepatocellular carcinoma, malignant glioma or cervical adenocarcinoma.
